(19) United States
(12) Patent Application Publication
(10) Pub. No.: US 2018/0187190 A1
(43) Pub. Date: Jul. 5, 2018

(54) NEW CRISPR ASSAYS

(71) Applicant: Erasmus University Medical Center Rotterdam, Rotterdam (NL)

(72) Inventor: Rogier Petrus Leonardus LOUWEN, Rotterdam (NL)

(73) Assignee: Erasmus University Medical Center Rotterdam, Rotterdam (NL)

(21) Appl. No.: 15/735,941

(22) PCT Filed: Jun. 10, 2016
(86) PCT No.: PCT/NL2016/050422

§ 371 Date: Dec. 12, 2017

(30) Foreign Application Priority Data
Jun. 12, 2015 (NL) PCT/NL2015/050438

(51) Int. Cl. C12N 15/113; G01N 33/50; C12N 15/85

(52) U.S. Cl. CPC C12N 15/113; G01N 33/5023; C12N 2310/14; C12N 2310/20; C12N 15/85

(57) ABSTRACT

The present invention relates to expression vectors harboring eukaryotic CRISPR sequences and their use in assays to study the intervention of eukaryotic CRISPR sequences with gene regulation and with gene editing in eukaryotes using CRISPR-Cas technology.

The invention relates to the field of genetics, more particular human genetics, more especially the use of eukaryotic CRISPR sequences in assays to study eukaryotic gene regulation through RNAi and in assays for studying the behavior of eukaryotic CRISPR sequences.

### INTRODUCTION

CRISPR Sequences are Clustered Regularly Interspaced Short

Palindromic Repeat sequences that are present in bacteria and archaea. Initially these kind of sequences have been indicated as Short Regularly Spaced Repeats (SRSRs) (Mojica, F. J. et al., 2000, Mol. Microbiol. 36:244-246), but they have been renamed in the acronym CRISPR by Jansen et al. (Jansen, R. et al., 2002, Mol. Microbiol. 43:1565-1575). Their function has been revealed later by Doudna and Charpentier, who independently from each other showed that CRISPR sequences work together with proteins from the Cas (CRISP associated) group to form a kind of immune reaction against viral infections (Pennisi, E., 2013, Science 341:833-836).

Together with Doudna and Charpentier, Jinek and others (Jinek, M. et al., 2012, Science 337:816-821) showed that Cas9, one of the Cas enzymes, in connection with an adapted version of the CRISPR (guide) sequences could be used for genetic editing. Since then the CRISPR-Cas system has been studied extensively and currently it is one of the most promising tools in genetic engineering because of its ease of use (e.g. Young, S. 2014, MIT Technol. Review: http://www.technologyreview.com/review/524451/genome-surgery/; Mali, P. et al., 2013, Nature Meth. 10:957-963).

Although the CRISPR-Cas system has been proven to work in eukaryotes for genetic editing, it has also been found that the system is not fail-proof. A very recent article (Cyranoski, D and Reardon, S., Nature News 22 Apr. 2015) demonstrated that, although Chinese scientists had been able to genetically engineer the genomes of human embryo's, in most of the embryo's that were treated the system did not work at all, or not successfully.

Accordingly, there is still insufficient information on? how the CRISPR-Cas system can be used in the eukaryotic cell and assays for studying the interaction of the gene editing sequences and the eukaryotic gene regulation are strongly needed.

### SUMMARY OF THE INVENTION

The present inventors now have surprisingly discovered that the occurrence of CRISPR sequences is not limited to bacteria and archaea, but that these sequences are also endogenous to eukaryotes. Especially in humans many CRISPR like sequences have been discovered. On basis of this finding, the invention now provides vector molecules comprising eukaryotic, preferably mammalian, more preferably human CRISPR sequences. Accordingly, the invention comprises a eukaryotic expression vector comprising a eukaryotic CRISPR, preferably a mammalian or a plant CRISPR. Preferably, said CRISPR is one of the human CRISPR sequences of SEQ ID NO: 1-SEQ ID NO: 10141 or any of the non-human CRISPR sequences of SEQ ID NO: 10142-11297. Further a eukaryotic expression vector according to the present invention preferably comprises a human CRISPR.

In a further embodiment the CRISPR is under control of an endogenous promoter. In an alternative embodiment the CRISPR is under control of a heterologous promoter.

Further part of the invention is a method of assaying the effect of a eukaryotic CRISPR on the gene regulation of a eukaryotic cell comprising the steps of:
a. transforming or transfecting said cell with a eukaryotic expression vector according to the invention;
b. allowing the CRISPR to be transcribed;
c. measuring the transcriptome of said cell.

Preferably in said method the CRISPR is a human CRISPR and said cell is a human cell. Further preferred is a method in which the effect on gene regulation is measured by comparing the total mRNA of the cell before and after expression of the CRISPR.

In a further embodiment the invention comprises a method of assaying the transcription of a eukaryotic CRISPR comprising the steps of:
a. transforming or transfecting a cell with a eukaryotic expression vector according to claim 4;
b. subjecting said cell to a stimulus;
c. measuring the transcriptome of said cell.

Preferably in this method according said stimulus is a chemical stimulus or a physical stimulus.

Also part of the invention is the use of a eukaryotic CRISPR for studying gene regulation in eukaryotes, preferably mammals, more preferably humans.

Preferably, said CRISPR is a human CRISPR sequence selected from SEQ ID NO: 1-SEQ ID NO: 11297, more preferably a humana sequence from any of SEQ ID NO: 1-10141.

The present invention further also comprises a kit comprising a vector according to the invention and instructions for use in a method according to the invention.

### LEGEND TO THE FIGURES

FIG. 1 Total number of human CRISPR blast hits visualized per taxonomic division. For both the human CRISPR repeats and spacers the number of BLAST hits were counted for each taxonomic division. False represents confirmed human CRISPRs and true represents questionable human CRISPRs. Taxonomic divisions include, Bacteria, Environmental samples, Invertebrates, Mammals, Phages, Plants, Primates, Rodents, Synthetic, Vertebrates and Viruses.

FIG. 2 ChIP seq data reveals specific transcription activity in human CRISPRs. CRISPRs are visualized in red blocks. The human reference genome that is used and uploaded into IGV is Hg19. IGV software is used to visualize the ChIP-seq data obtained from ENCODE for the cell lines U2OS, Caco-2 and K562. A) ChIP seq data for human CRISPR with SEQ ID:4443 shows in an intergenic region at position (chr7:99,190,443-99,190,629) transcription activity in U205,

Caco-2, and K562 cell lines; B) ChIP seq data for human CRISPR with SEQ ID: 5838 shows in an intronic region at postion (chr10:70,276,886-70,277,044) transcription activity in U2OS and Caco-2 cell lines, with transcripts reverse complementary orientated to the RNA transcript of gene SLC25A16; C) ChIP seq data for human CRISPR with SEQ ID: 189 shows in an exonic region at postion (chr1:45,965,017-45,965,162) transcription activity in the cell lines U2OS, Caco-2 and K562, with transcripts that are reverse complementary orientated to the mRNA transcript of gene CCDC163P; D) ChIP seq data for human CRISPR with SEQ ID: 8204 shows in an intronic region at postion (chr17:19,149,168-19,149,358) transcription activity in the cell line U2OS, with transcripts that are reverse complementary orientated to the RNA transcript of gene EPN2; E) ChIP seq data for human CRISPR with SEQ ID: 4038 shows in an intronic region at postion (chr6:167,093,513-167,093,713) transcription activity in the cell lines U2OS and K562, with transcripts that are reverse complementary orientated to the RNA transcript of gene RPS6KA2; F) ChIP seq data for human CRISPR with SEQ ID: 5213 shows in an intronic region at postion (chr9:8,644,153-8,644,333) transcription activity in the cell lines Caco-2 and K562, with transcripts that are reverse complementary orientated to the RNA transcript of gene PTPRD.

FIG. 3 Human body map RNA-seq data reveals tissue specific human CRISPR transcription activity. CRISPRs are visualized in red blocks. The human reference genome that is used and uploaded into IGV is Hg19. IGV software is used to visualize the Body Map 2.0 (Illumina HiSeq) RNA-sequence data of tissues Brain, Colon, Heart, Kidney, Liver, Lung, Skeltal muscle, Thyroid, White blood cell, Adrenal, Lymph node, Ovary, Testes, Adipose, Breast and Prostate and is visualized in blue. A) RNA-seq data for human CRISPR with SEQ ID: 2296 shows in an intronic region at position (chr4:15,617,172-15,617,283) transcription activity in tissues Brain and Heart; B) RNA-seq data for human CRISPR with SEQ ID: 2367 shows in an intronic region at position (chr4:36,322,560-36,322,655) transcription activity in tissues Lung and Lymph node; C) RNA-seq data for human CRISPR with SEQ ID: 3247 shows in an intergenic region at position (chr5:98,280,615-98,280,721) transcription activity in tissues Colon, Heart, Thyroid and Ovary; D) RNA-seq data for human CRISPR with SEQ ID: 4311 shows in an intronic region at position (chr7:70,215,373-70,215,559) transcription activity in tissues Thyroid and Adrenal; E) RNA-seq data for human CRISPR with SEQ ID: 4397 shows in an intergenic region at position (chr7:99,188,869-99,189,616) transcription activity in tissues Brain, Kidney, Testes and Adipose; F) RNA-seq data for human CRISPR with SEQ ID: 9451 shows in an intergenic region at position (chrX:1,007,571-1,007,935) transcription activity in tissues Colon, Kidney, Thyroid and Testes.

FIG. 4 Twelve examples of human CRISPR expression vectors. Sequences from example 4 were uploaded into Snapgene viewer, which is a versatile tool to create annotated sequence files in a vector map format. This is done for the human CRISPR sequences that recede in ADAM10, ADAM17, ADAMTS9-AS2, TUBD and IL-10. The vectors contain an U6 promoter and a transcription termination signal, the human CRISPR sequence were generated in such a way that the expression vector would generated transcripts in a Forward and Reverse complementary manner.

FIG. 5 Human CRISPR vectors control gene expression. A) Human CRISPR vector pLOHA_7710_+ downregulates ADAM10 expression in U2OS cells 24-48 hours after transfection. For each plasmid pLOHA_7710_+ or pLOHA_7710_−, pCDNA3.1 transfected and untreated cells three representable pictures are shown. U2OS cells were stained for ADAM10 visualized in red and the nuclei were stained with DAPI. Pictures were taken at a 40× magnification using the Olympus XI51 microscope; B) and C) Human CRISPR vector pLOHA 1762− induces ADAMTS9 expression by silencing the ADAMTS9-AS2 antisense RNA. For each plasmid pLOHA_1762_+ or pLOHA_1762_−, pCDNA3.1 transfected and untreated cells three representable pictures are shown. U2OS bone marrow epithelial cells and SKBR2 breast cancer epithelial cells were stained for ADAMTS9 (visualized in red) and the nuclei were stained with DAPI. Pictures were taken at a 40× magnification using the Olympus XI51 microscope. D) Western blot of U2OS total cell lysates that were transfected with pLOHA_7710_+ or pLOHA_7710_−, pCDNA3.1 or left untreated. Expression differences of ADAM10 were detected between 55 and 70 kDa.

FIG. 6 Human CRISPRs and Cas9 induce toxic double stranded DNA breaks in U2OS cells. U2OS cell were infected with C. jejuni strain GB11, GB11Acas9, GB11Acas9A, untreated (NC), 1Gy radiated for the induction of DSB (PC) or pCDNA3.1+CjCas9 (GB11) transfected. BLESS identified break position is visualized as a blue box; A) shows a CjCas9 dependent DSB break position that is induced by GB11, GB11Δcas9Δ and CjCas9 at the exact same position visualized in region (chr5:57,182,990-57,183,030) for which the functional human CRISPR guide with patent seq ID 115 was required; B) shows a CjCas9 dependent DSB break position that is induced by GB11, GB1lAcas9A and CjCas9 at the exact same position visualized in region (chr1:237,600,585-237,600,625) for which the functional human CRISPR guide with SEQ ID 1471 was required. This human CRISPR guide is actively transcribed in U2OS cells at position chr2:219072954-219073050 under standard cell culture conditions; C) shows a CjCas9 dependent DSB break position that is induced by GB11, GB11Acas9A and CjCas9 at the exact same position visualized in region (chr17:4,861,104-4,861,144) for which the functional human CRISPR guide with SEQ ID 1109 was required. This human CRISPR guide is actively transcribed in U2OS cells at position chr2:85737752-85738008 under standard cell culture conditions; D) shows a CjCas9 dependent DSB break position that is induced by GB11, GB11Acas9A and CjCas9 at the exact same position visualized in region (chr12:14,461,527-14,461,567) for which the functional human CRISPR guide with SEQ ID 130 was required. This human CRISPR guide is actively transcribed in U2OS cells at position chr1:28173679-28173782 under standard cell culture conditions; E) shows a CjCas9 dependent DSB break position that is induced by GB11, GB11Acas9A and CjCas9 at the exact same position visualized in region (chr10:33,287,520-33,287,560) for which the functional human CRISPR guide with SEQ ID 2750 was required. This human CRISPR guide is actively transcribed in U2OS cells at position chr4:163803118-163803209 under standard cell culture conditions.

FIG. 7 Effect of medicines, chemicals or biological agents on CRISPR expression. Four affymetrix probes 1557645_at, 1560498_at, 1556520_at, 1559278_at that matched exactly with the human CRISPR SEQ ID 1884; 7953; 2700 or 4812, respectively, were analysed in http://www.ncbi.nlm.nih.gov/geoprofiles/. Expression dataset pictures were copied to a word file when significant expression differences were observed between controls and (a)biotic compounds or medicine exposed cell lines or subjects.

### DETAILED DESCRIPTION OF THE INVENTION

The structure of a prokaryotic CRISPR array (see e.g., Horvath, P. and Barrangou, R., 2010, Science 327:167-170) includes a number of short repeating sequences referred to as “repeats.” The repeats occur in clusters and up to 249 repeats have been identified in a single CRISPR array and are usually regularly spaced by unique intervening sequences referred to as “spacers.” Typically, CRISPR repeats vary from about 24 to 47 base pairs in length and are often? palindromic. The repeats are generally arranged in clusters (up to about 20 or more per genome) of repeated units. The spacers are located between two repeats and typically each spacer has a unique sequence of about 21-72 base pairs in length. Many spacers are identical to or have high similarity with known phage sequences. It has been shown that the insertion of a spacer sequence from a specific phage into a bacterial CRISPR can confer resistance to that phage (see e.g., Barrangou, R. et al., 2007, Science 315:1709-1712).

In addition to repeats and spacers, a CRISPR array may also include a leader sequence and often a set of two to six associated cas genes. Typically the leader sequence is an AT-rich sequence of up to 550 base pairs directly adjoining the 5′ end of the first repeat. New repeat-spacer units are almost always added to the CRISPR array between the leader and the first repeat.

The present inventor discovered CRISPR sequences in eukaryotes that follow a similar genetic make-up as the prokaryotic CRISPRs: short repeating, often palindromic sequences of 24-47 base pairs separated by spacers of—generally—21-72 base pairs. A number of such eukaryotic CRISPR sequences is depicted in Table IA-IX of the priority document PCT/NL2015/050438, now presented as SEQ ID NO: 1-10000, (Table IA covers chromosome 1, table IB covers chromosome 2, etc.) or in Table IIA-IIM of said priority document, now presented as SEQ ID NO: 10142-11297 in which non-human eukaryotic CRISPRs are depicted. New in this application are the CRISPR sequences of SEQ ID NO: 146-149, 545-554 and 10001-10141. These CRISPRs occasionally are found to be accompanied by a Cas gene, sometimes even more than one Cas gene.

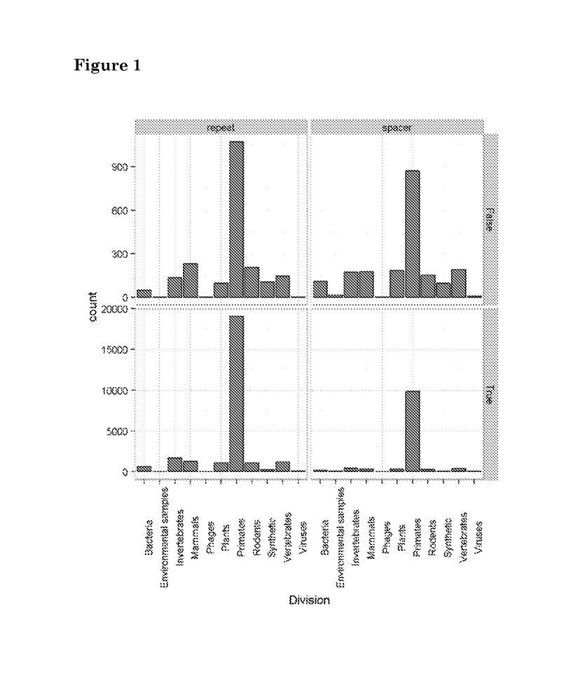

In the present application a eukaryotic CRISPR sequence is defined as a sequence that comprises at least two partly or complete palindromic repeats of 24-47 base pairs and at least one spacer of about 21-72 base pairs, wherein the spacer is derived from a eukaryotic sequence, especially a spacer sequence that is derived from the same organism as from which the CRISPR sequence is derived. The spacer may originally be derived from a non-eukaryotic pathogen, but it will be different from the non-eukaryotic sequence because of the connection with the repeat sequences which are of eukaryotic origin. According to this definition a human CRISPR sequence would be a CRISPR sequence in which the spacer contains a human sequence or a sequence of a human pathogen (such as a retrovirus (HERV)), but of which the repeat sequences are of human origin. A specific group of eukaryotic CRISPRs are those CRISPRS that comprise spacer sequences that are only consisting of sequences that are derived from the same organisms as from which the repeat sequences are derived (see also FIG. 1). These are indicated in the present application as “pure eukaryotic” CRISPR sequences. Accordingly a “pure human CRISPR sequence” is a pure eukaryotic sequence that is derived from a human being. The spacer of such a pure eukaryotic CRISPR generally is directed against a eukaryotic target sequence in such a sense that it will be capable of binding to such a sequence. It should further be mentioned that the spacer sequence does not need to be completely identical to the (eukaryotic) target. As has been proven in the work on RNAi (see below) inhibition of expression can also be accomplished with sequences that are less than 100% complementary to their target sequence. Because of the occurrence of mutations within the spacer sequences, which are more vulnerable to mutations than sequences coding for functional proteins, it could be that the original 100% complementarity has become lost. Nevertheless, it appeared that the spacer sequences that the present inventors have found in the CRISPR sequences of eukaryotic organisms are highly homologous with endogenous sequences (i.e. sequences of the same organism) (see FIG. 1?).

The CRISPR sequences that are listed in the Tables IA-IX and Tables IIA-IIM of the priority document PCT/NL2015/050438, and which with some additions are now presented as SEQ ID NO: 1-11297, can been found by using the CRISPR Finder software (CRISPRFinder: a web tool to identify clustered regularly interspaced short palindromic repeats. Nucleic Acids Res. 2007 May 31). The program for this should be run with the following standard default parameters: a repeat length of 23 to 55 bp, a gap size between repeats of 25 to 60 bp, one nucleotide mismatch between repeats, but these parameters may be varied in an advanced search to obtain additional CRISPRs. Further criteria a CRISPR should fit to are the following:

The spacer size compared to the DR size: The CRISPRfinder filter is mainly added to eliminate structures having for example a 45 bp DR and a 20 bp spacer. By default, the spacer size should be from 0.6* to 2.5* the DR size.

The spacers' similarity: This filter is set to eliminate tandem repeats. The spacers' comparison is made by aligning them (using default parameters of the ClustalW program). Spacers' similarity percentage is calculated with the function percentage_identity( )of the (Bio)perl interface (AlignIO methods, ClustalW interface; Larkin, M. A. et al., 2007, Bioinformatics 23:2947-2948). By default, this parameter is set to 60%.

The DR conservation: The direct repeat should be well conserved. The DR scan is done using the fuzznuc program of the EMBOSS package (Rice, P. et al. 2000, Trends in Genetics 16:276-277). The allowed mismatch is equal to one-third of the DR size (default parameters) to take into account the degenerated DR (one of the flanking DRs). Then a global mismatch score is computed as the average of mismatches (not including the degenerated DR) and this score should not exceed a threshold of 20% of the DR size(by default).

It is submitted that for the human CRISPR sequences the sequences covered in SEQ ID NO: 1-10141 are the total of sequences that may be discovered by screening the genome that is contained in the database. In the present finding the genomes of Homo sapiens present in the Bioconductor packages was used comprising data of about 1000 human genomes that can be retrieved from http://bioconductor.org/packages/BSgenome.Hsapiens.1000genomes.hs37d5/. It is submitted that further human CRISPR sequences may be retrieved from human genomic sequences that are different from the sequences that have been assessed for the current collection of CRISPR sequences. Further, examples have been given for further sequences from eukaryotic organisms in SEQ ID NO: 9987-1142. This collection is of course by no means complete: for all species mentioned only a few CRISPR sequences have been listed and it is certain that other eukaryotic species and genera will also harbor numerous CRISPR sequences when assessed with the CRISPRfinder algorithm as described herein.

As can be seen from the sequence listing, many eukaryotic CRISPRs have only two repeats, but there are sequences which contain more than 30 repeat sequences and there is even one at human chromosome 19 with more than 150 repeat sequences. Some of the repeat sequences are recognized by the CRISPR Finder software as repeat sequences that are identical or very similar to repeat sequences that also occur in bacteria or archaea (see FIG. 1), but in most cases the repeat sequences, although fulfilling the criteria set for the detection of CRISPR repeat sequences, seem to be unique. As far as currently investigated there does not seem to be any specific feature of the eukaryotic CRISPR repeat sequences that would clearly distinguish them from prokaryotic or archaeal CRISPR repeat sequences, except of the course for the fact that most of the repeat sequences are unique for the eukaryotic source and no homologous sequences are thus far found in bacteria or archaea (see FIG. 1).

Considering the source of the eukaryotic CRISPRs it seems that in some cases they could be hypothesized to have entered the eukaryotic genome from horizontal gene transfer (by whatever mechanism) from mainly protists (obtained from data from Crisp, A. et al. 2015, Genome Biol. doi: 10.1186/s13059-015-0607-3) although to a lesser extent also from bacteria, plants and viruses. In other cases, however, eukaryotic CRISPRs appear to have spacer sequences that are derived from viruses or other pathogens that are known to infect eukaryotic cells. In this respect it thus appears that these CRISPRs have been established through a similar mechanism as is described for bacteria and archaea. However, in the majority of the cases, the spacers are derived from sequences that are endogenous to the organism in which the CRISPR sequence is found or has been obtained from through horizontal gene transfer.

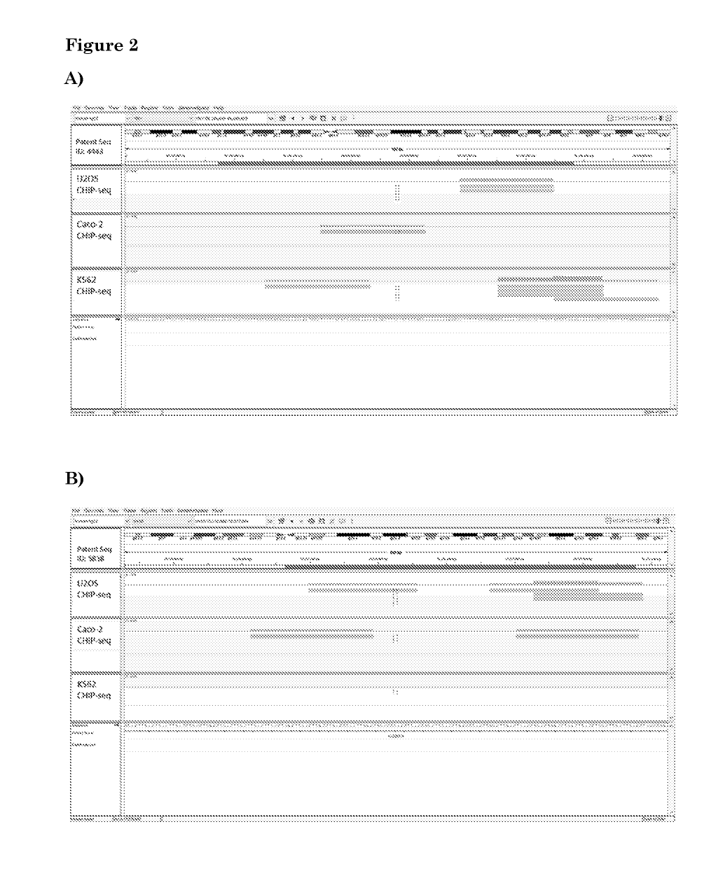

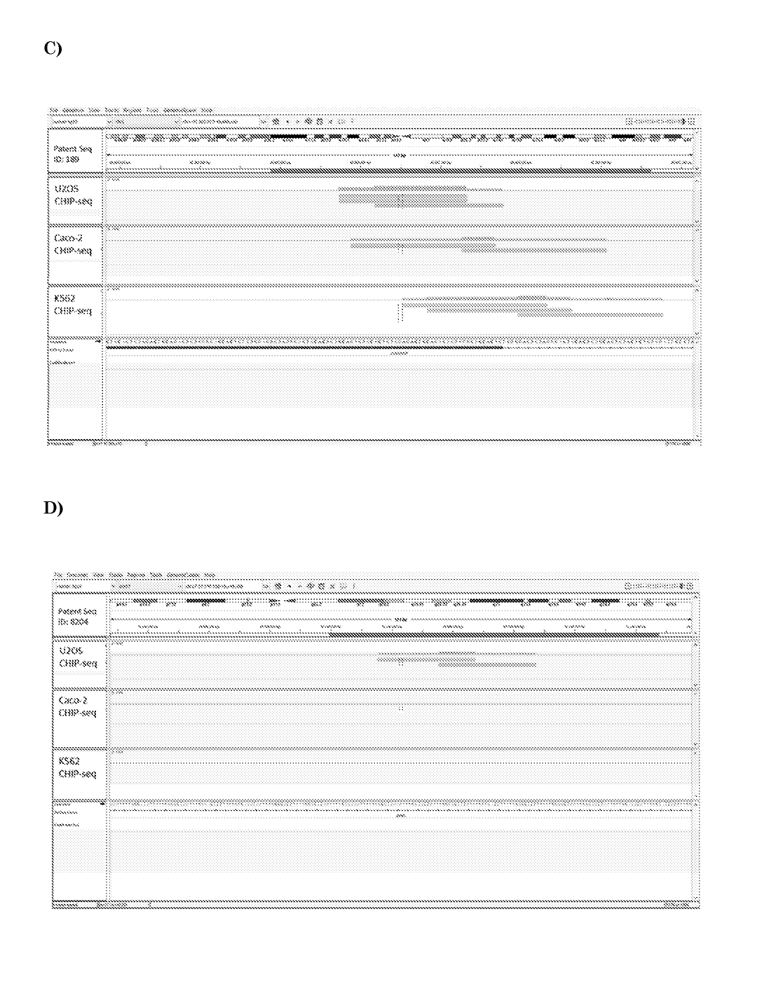

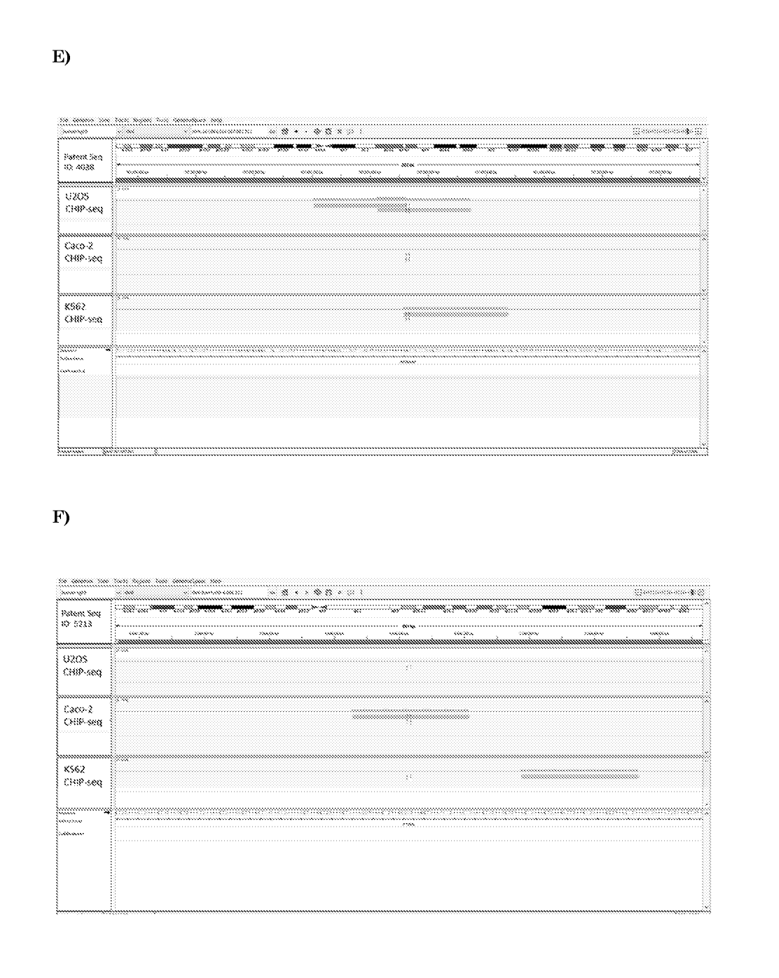

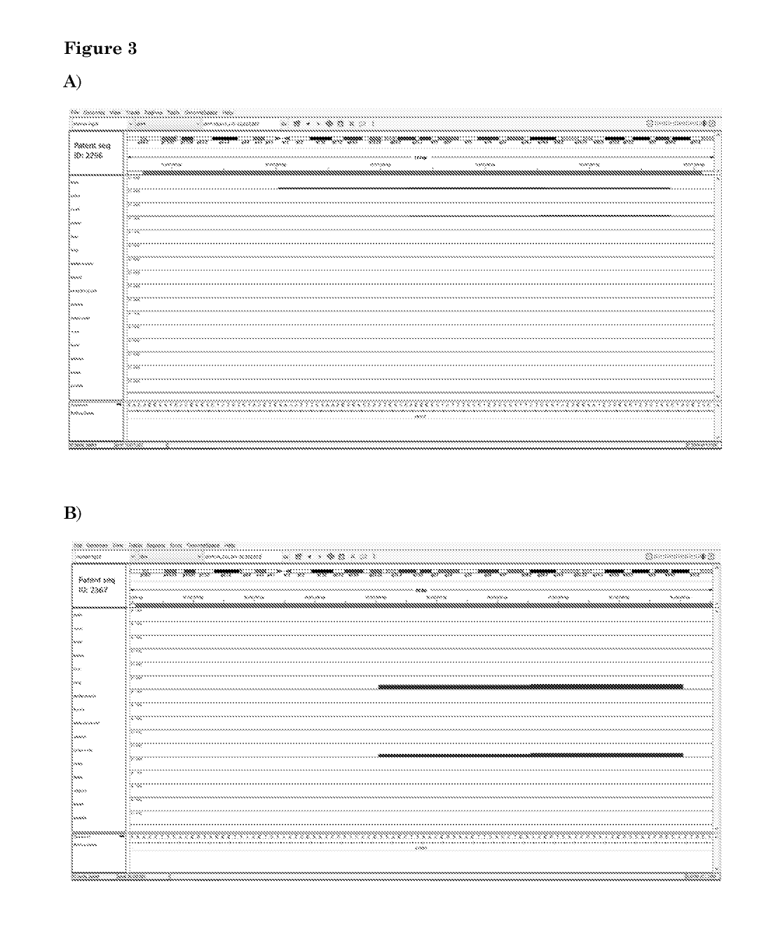

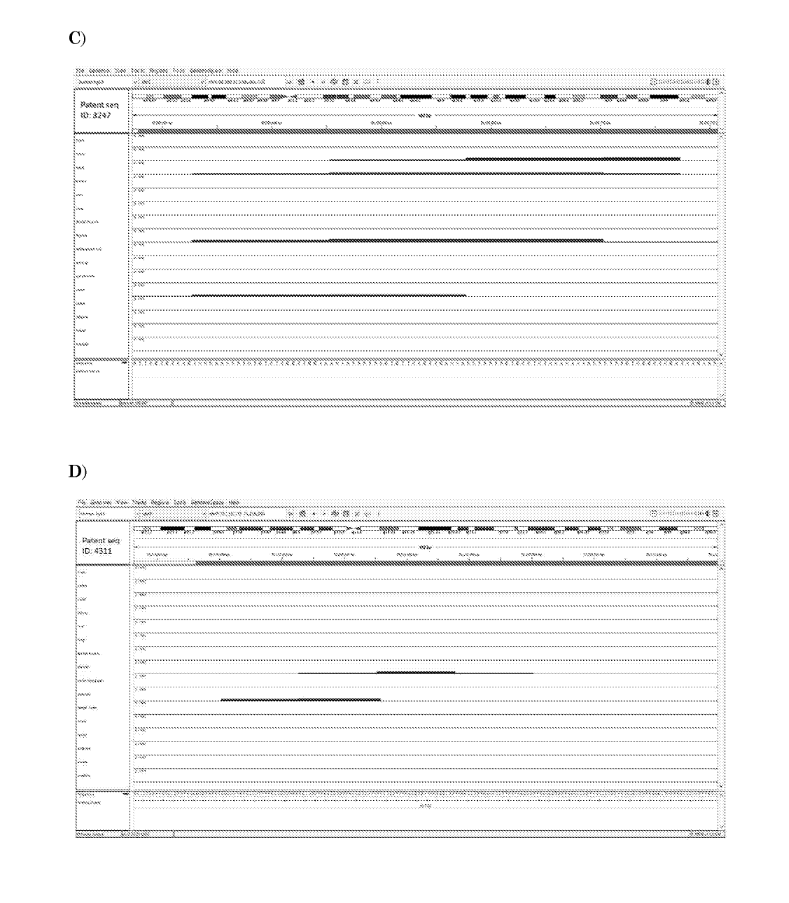

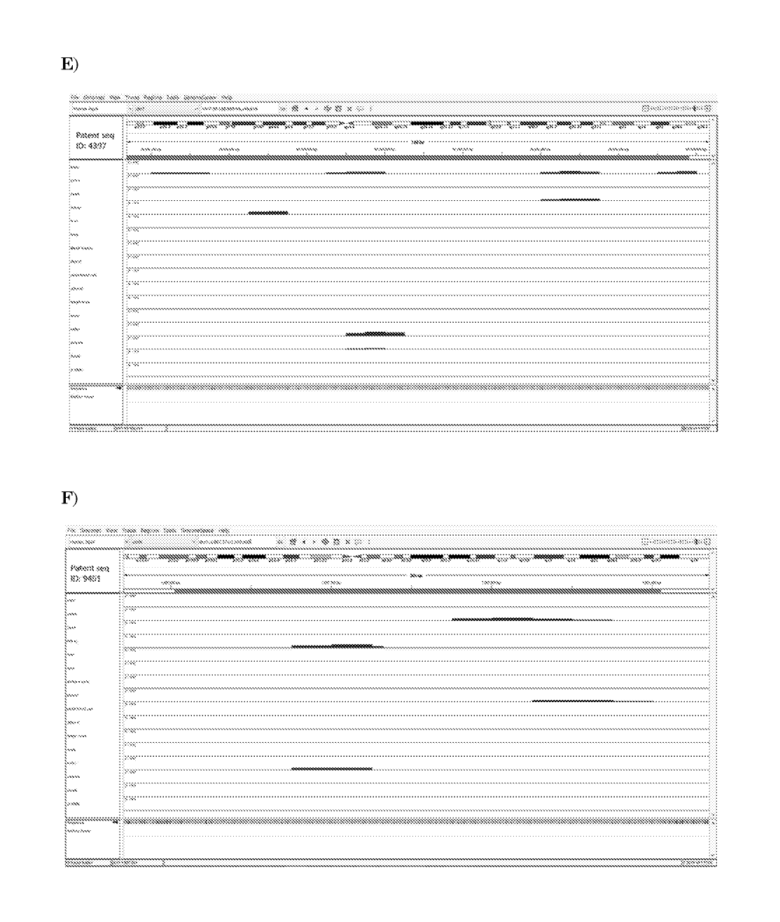

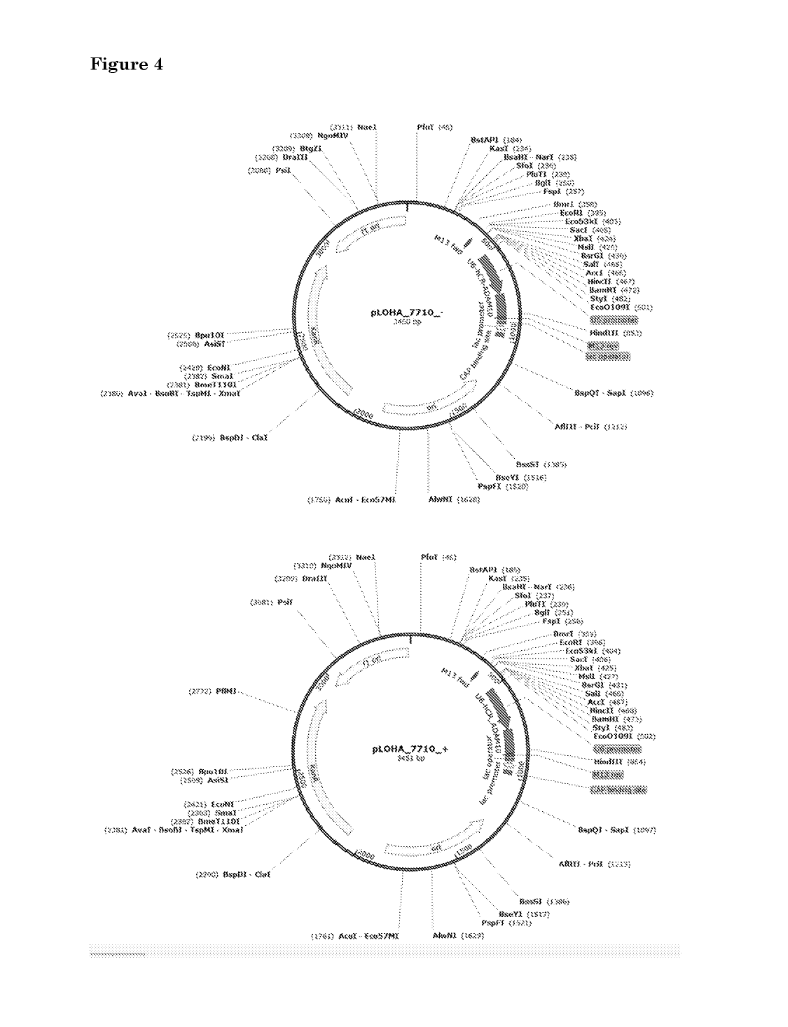

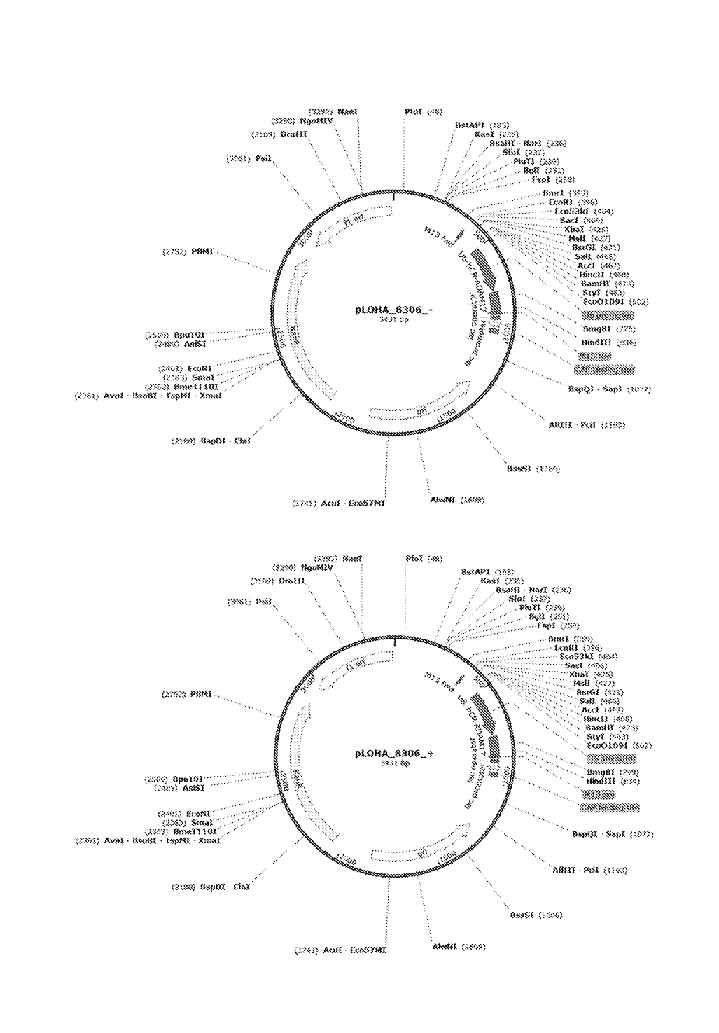

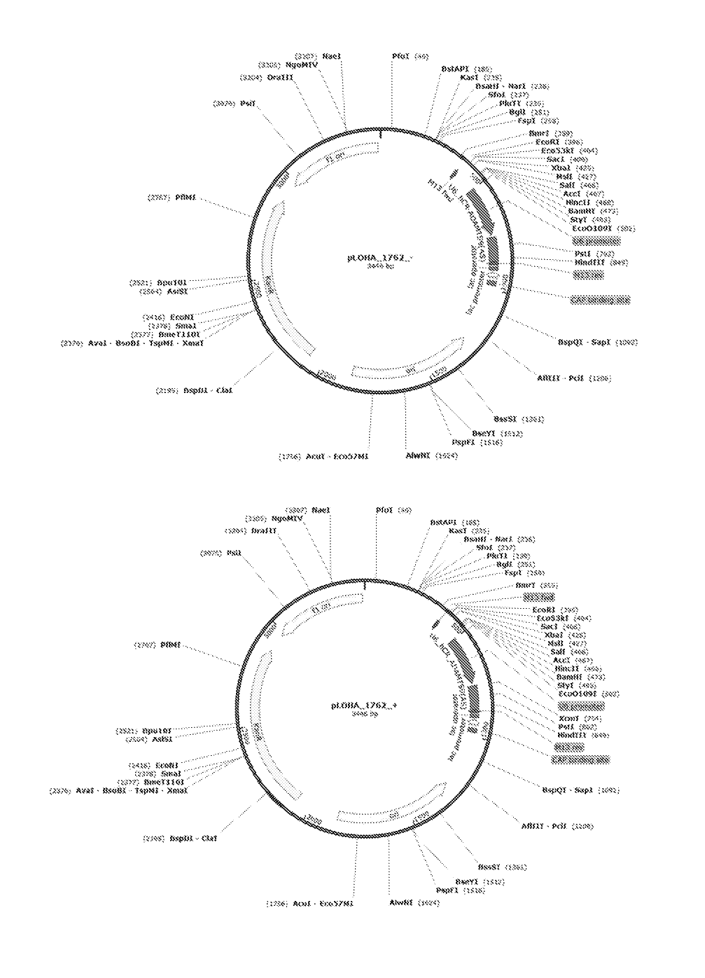

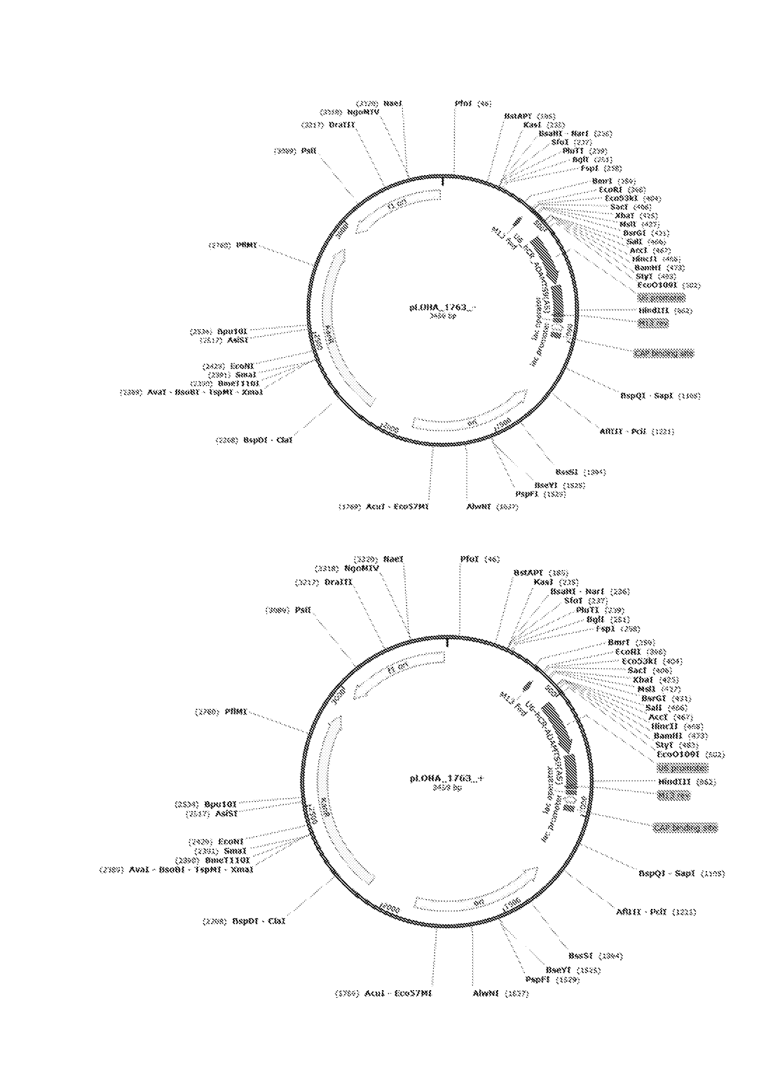

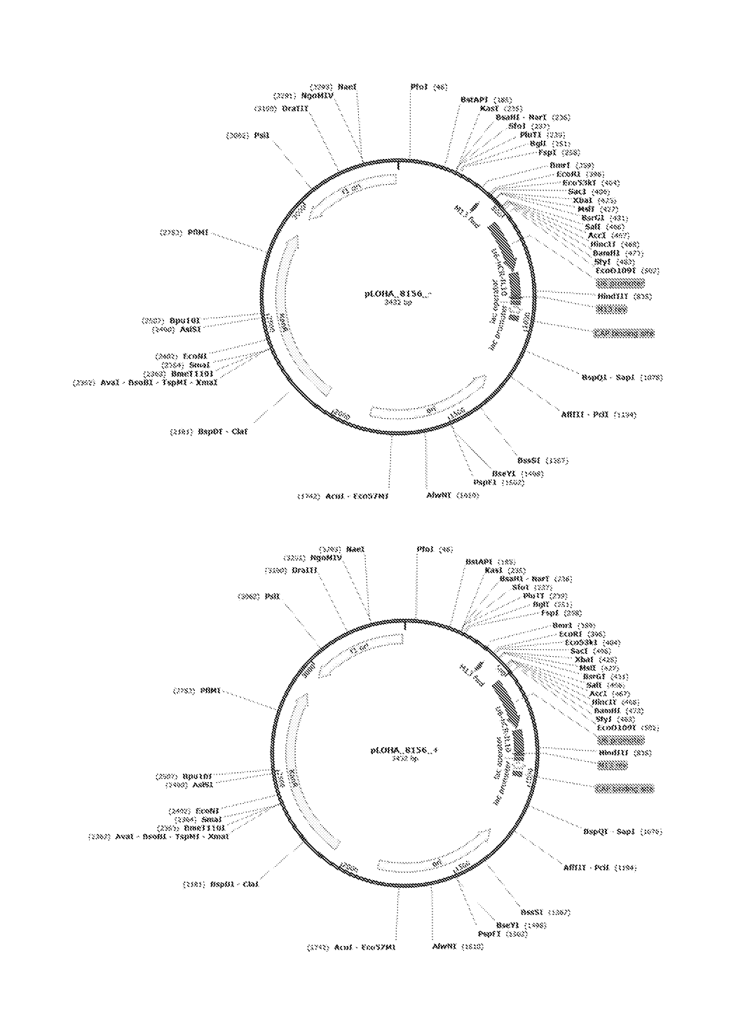

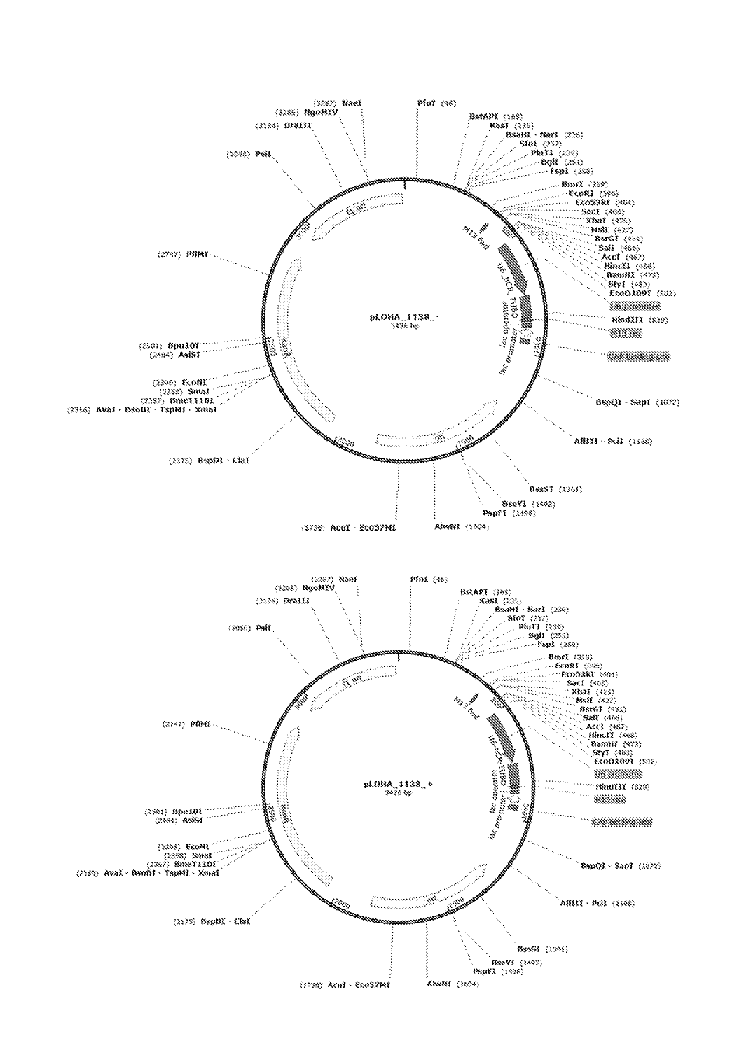

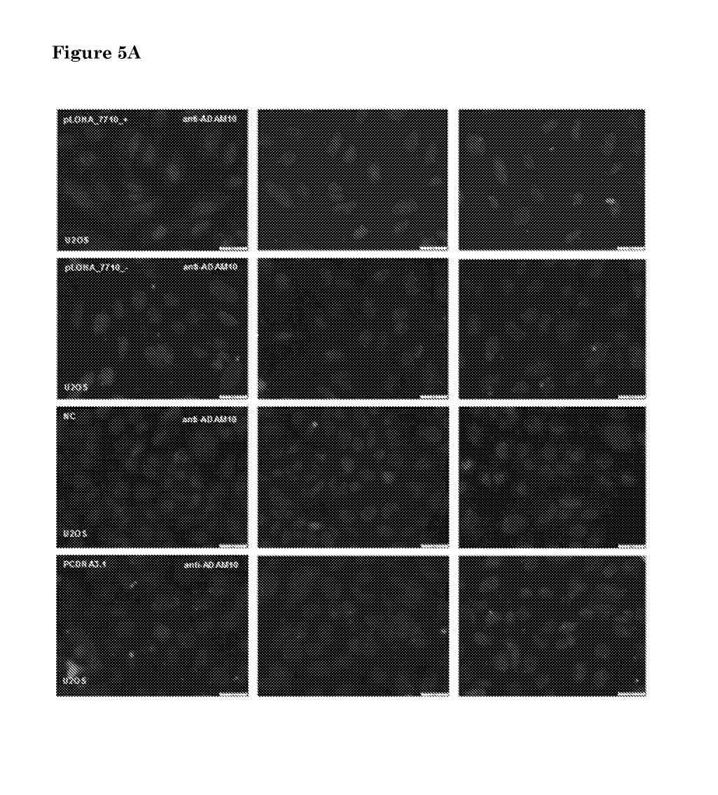

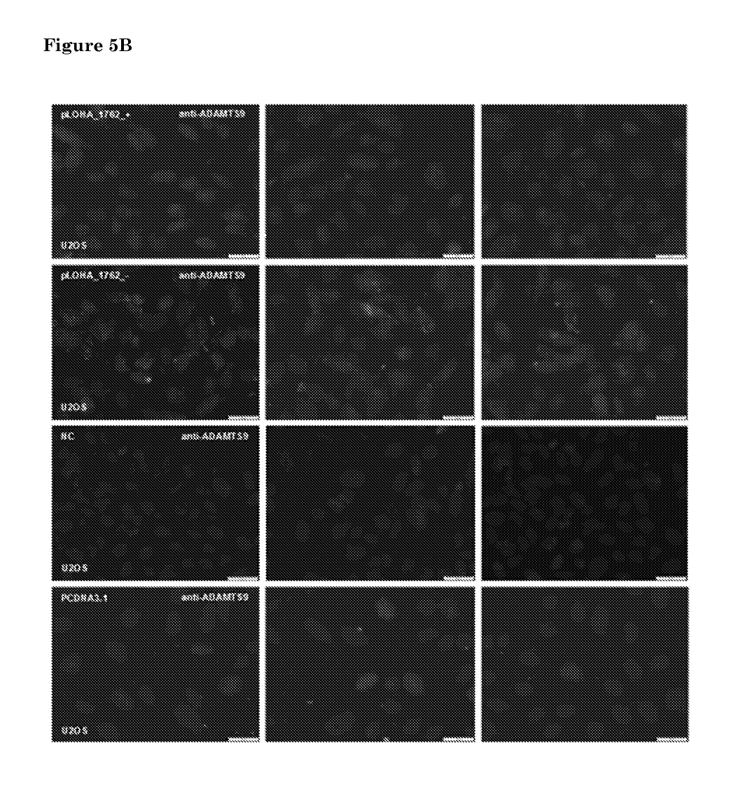

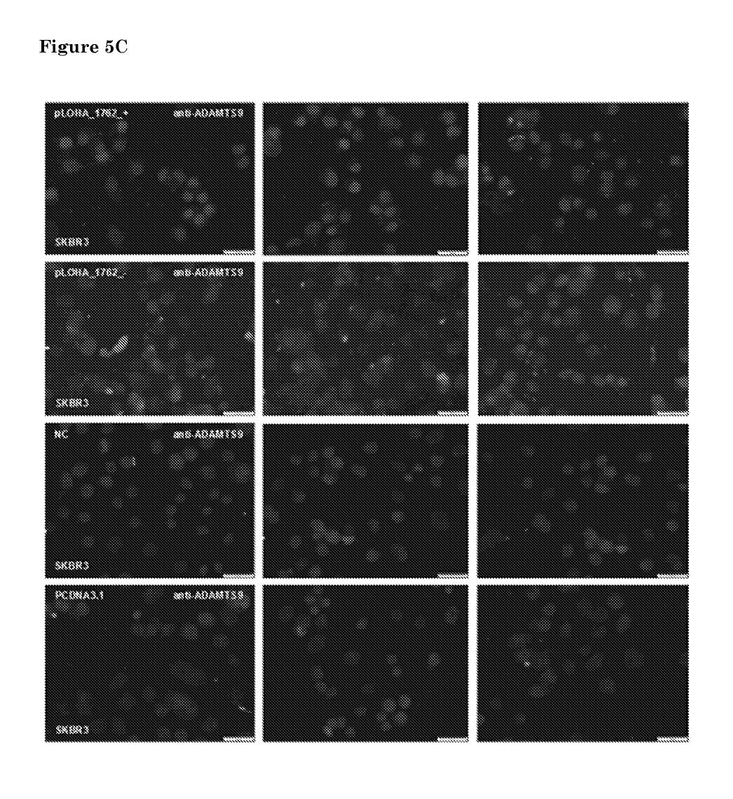

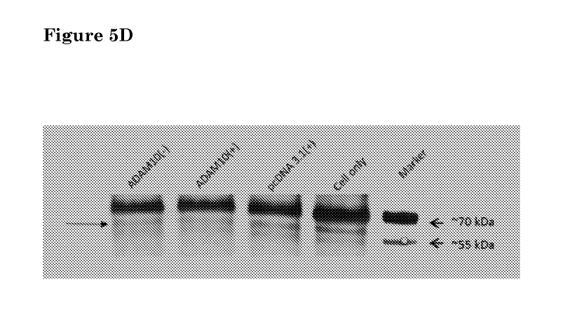

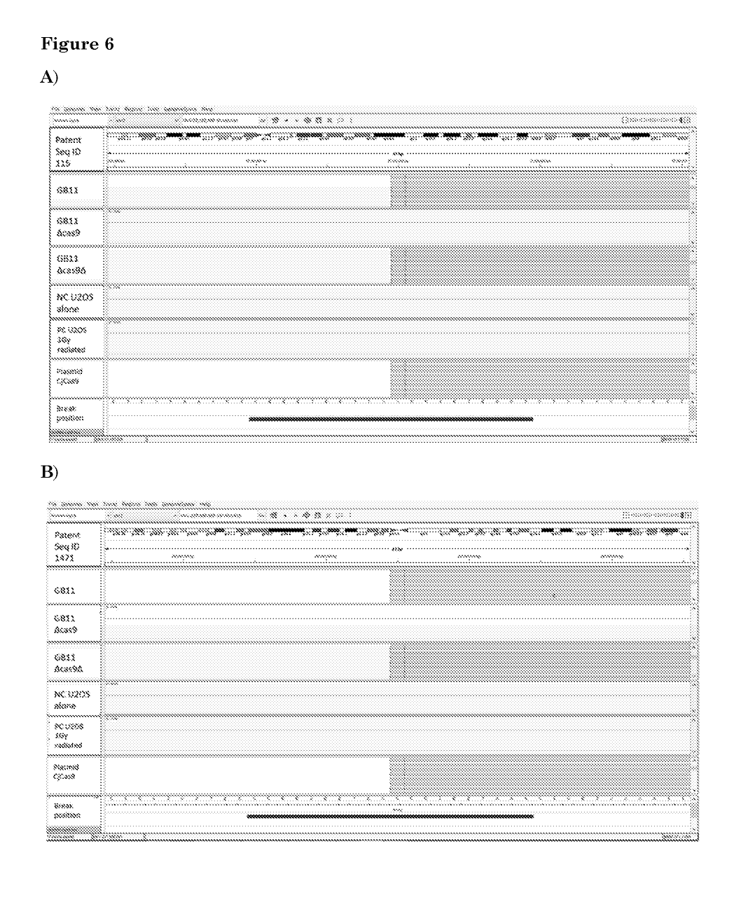

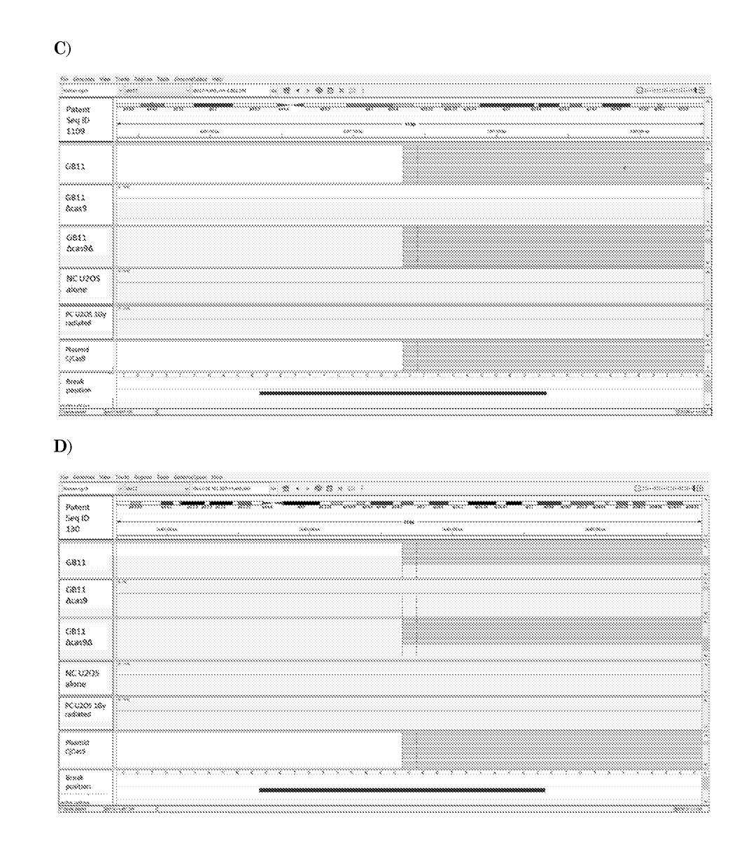

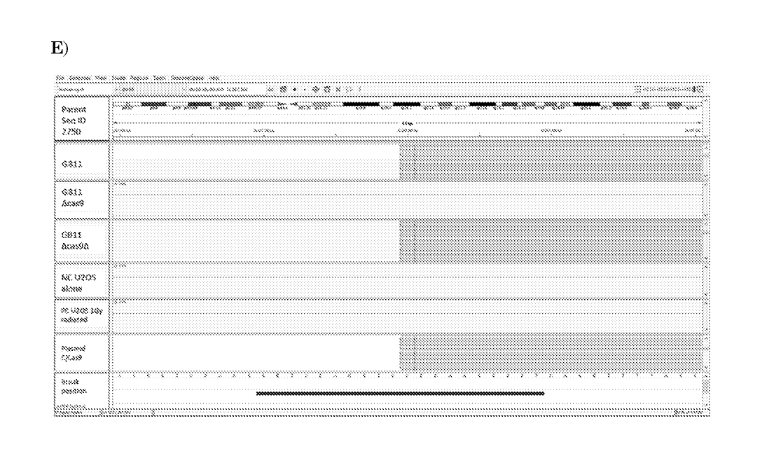

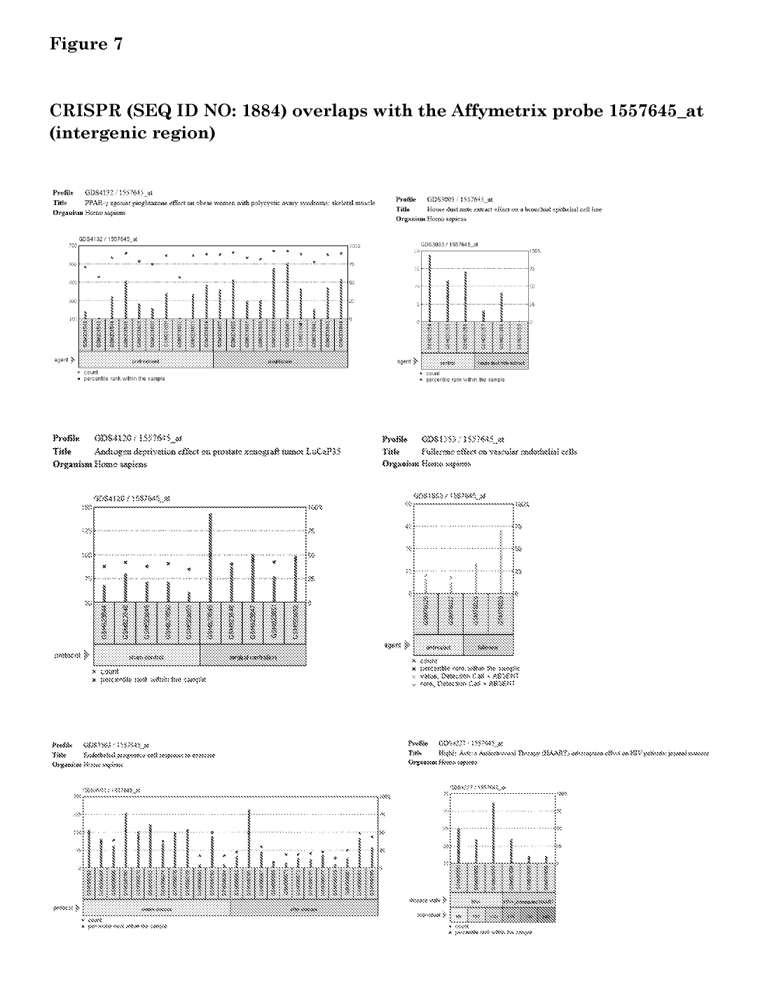

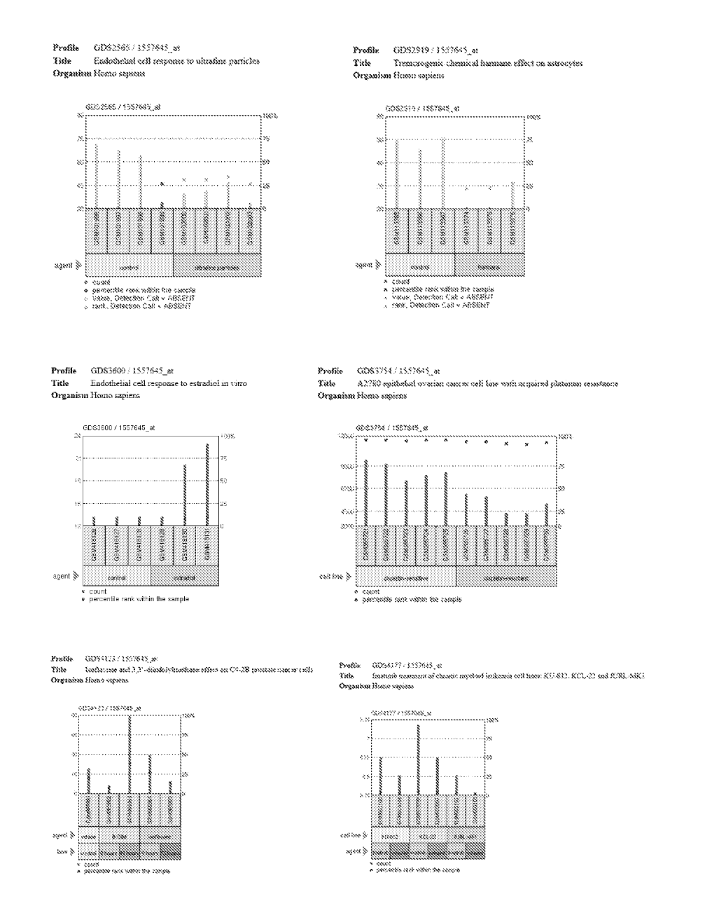

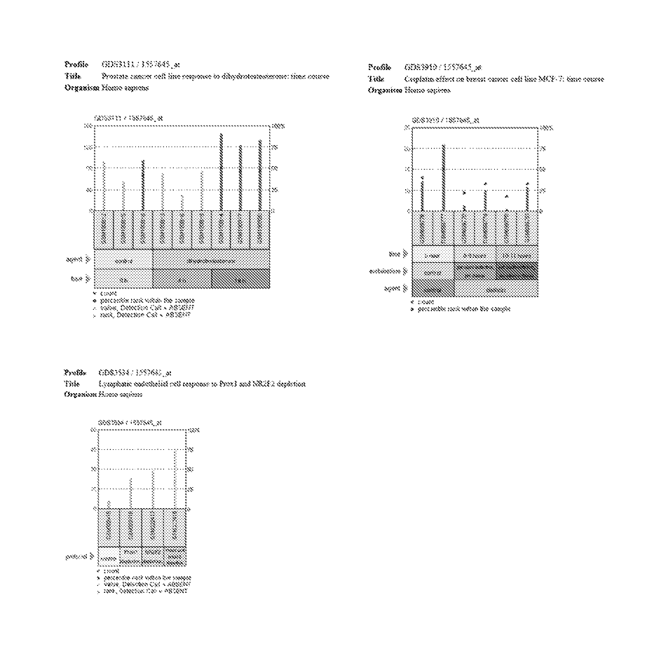

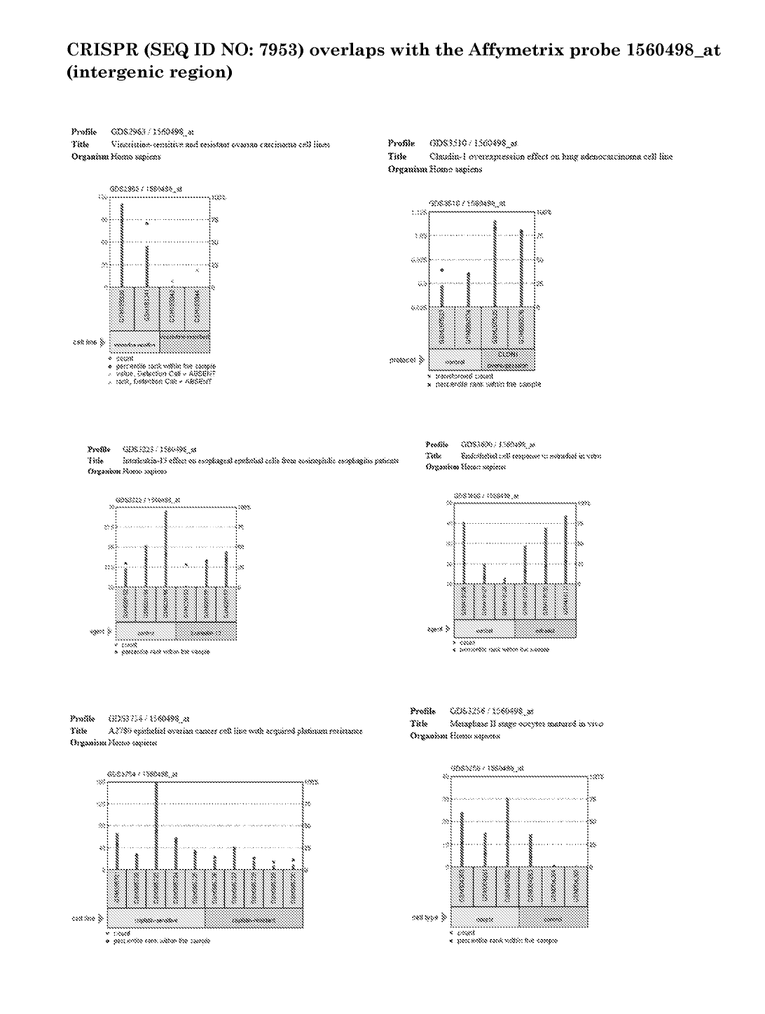

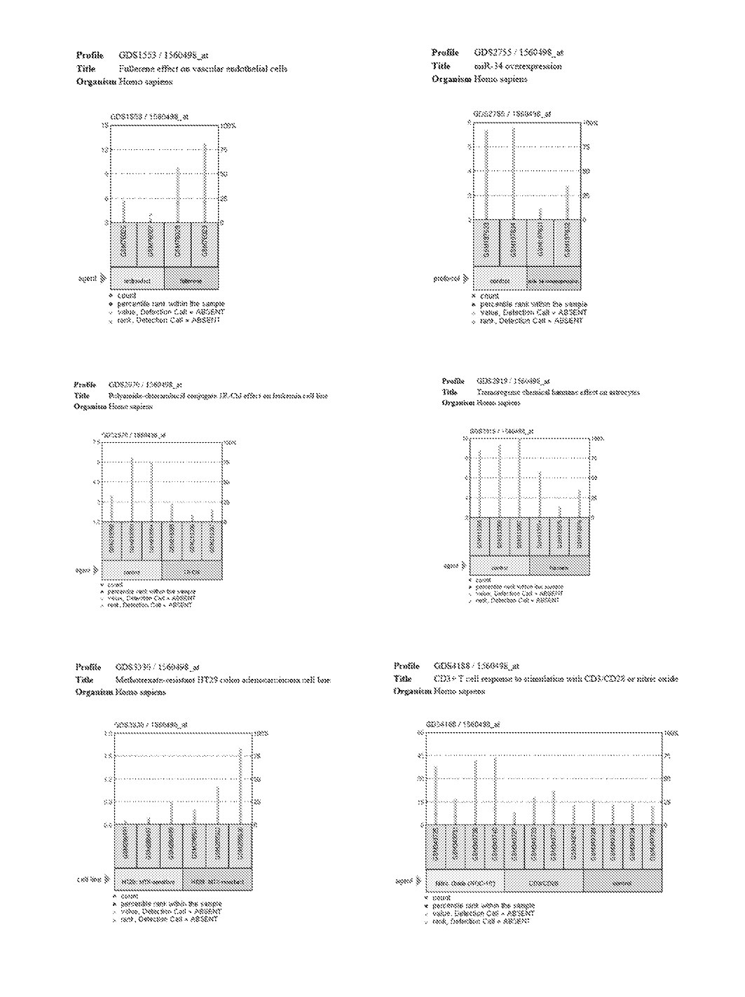

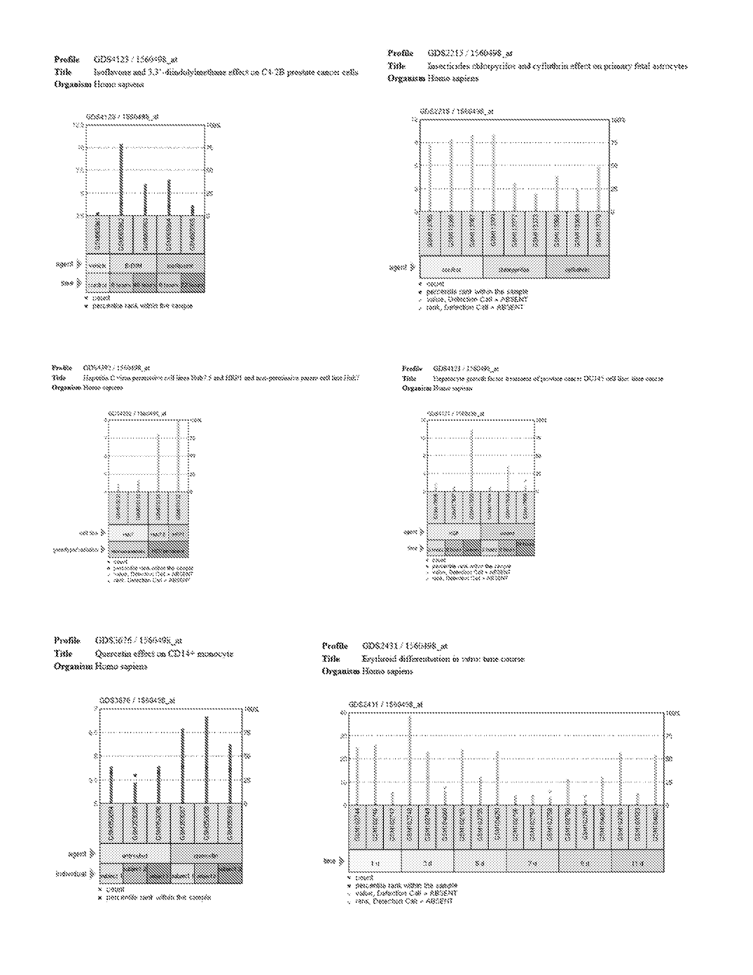

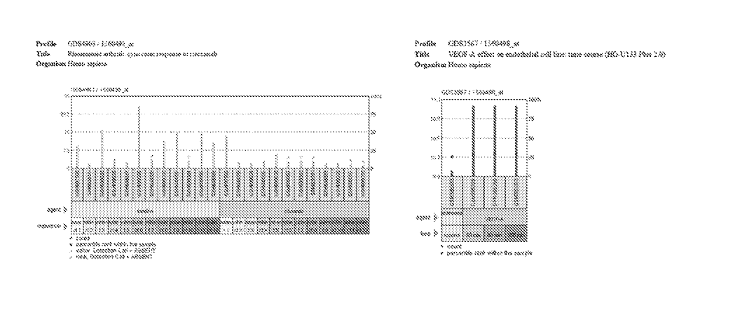

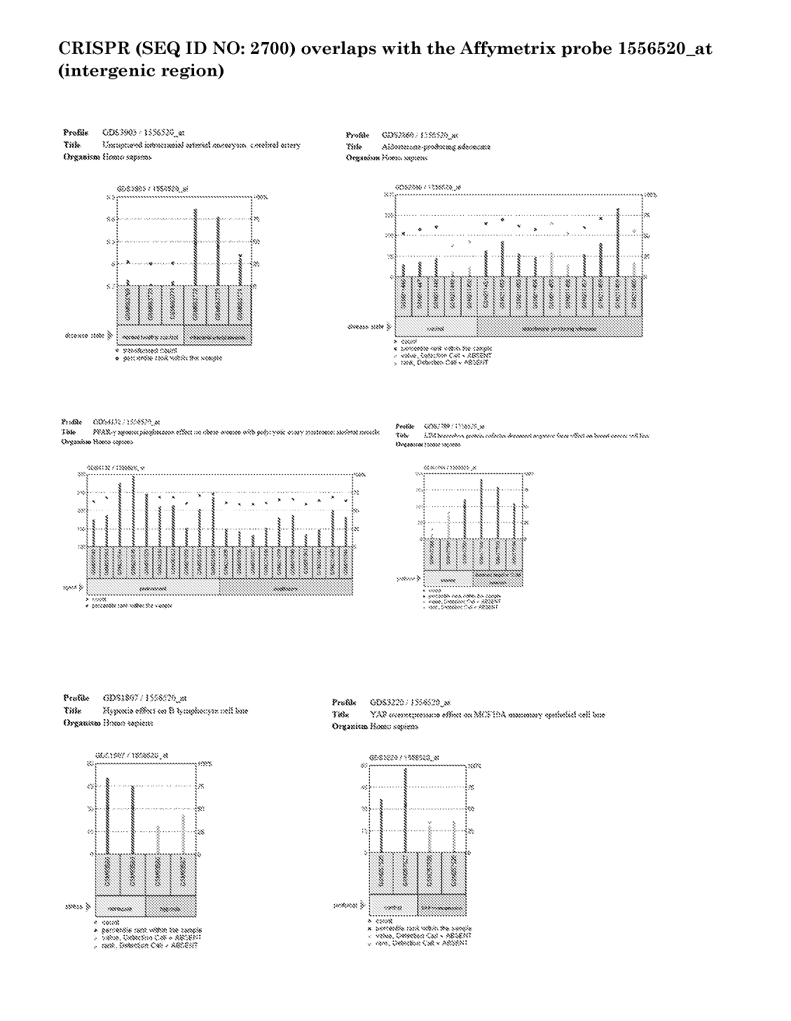

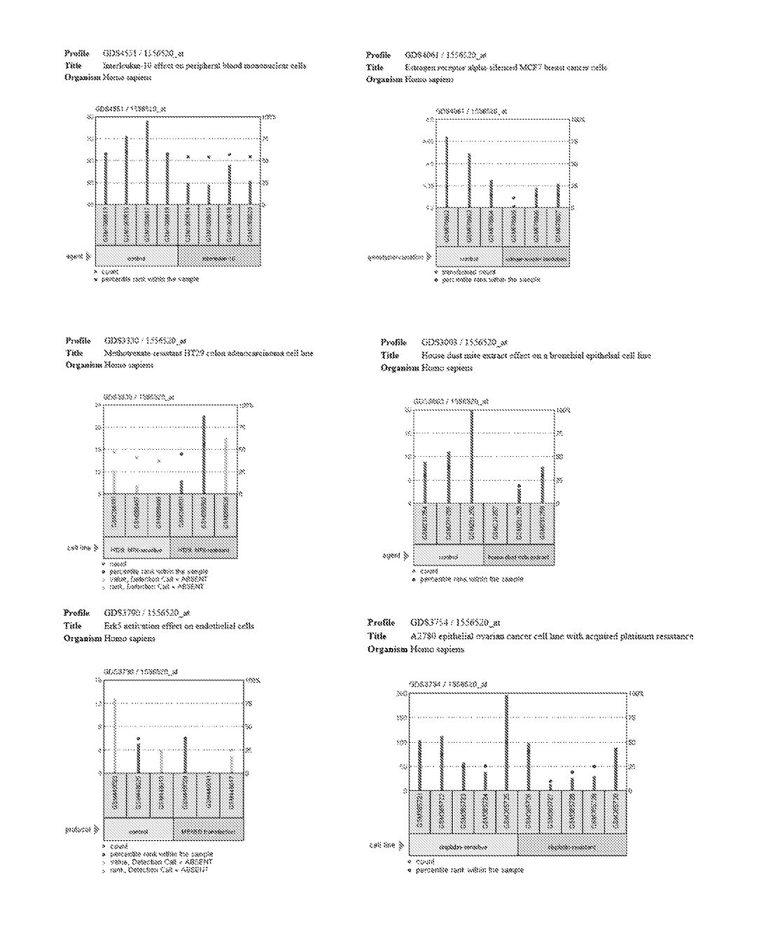

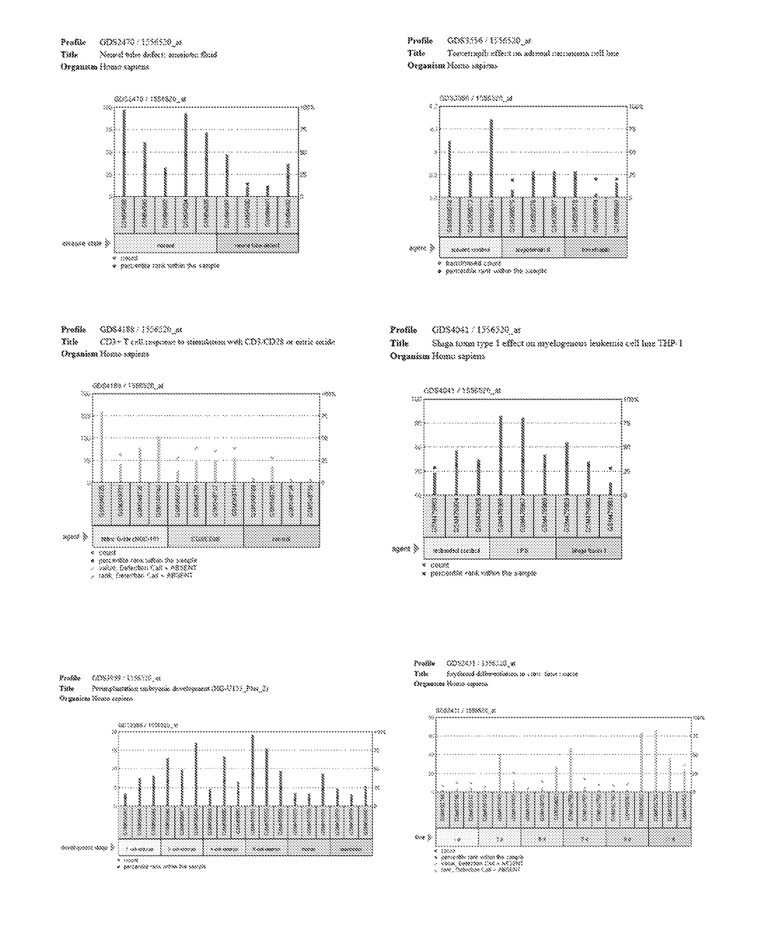

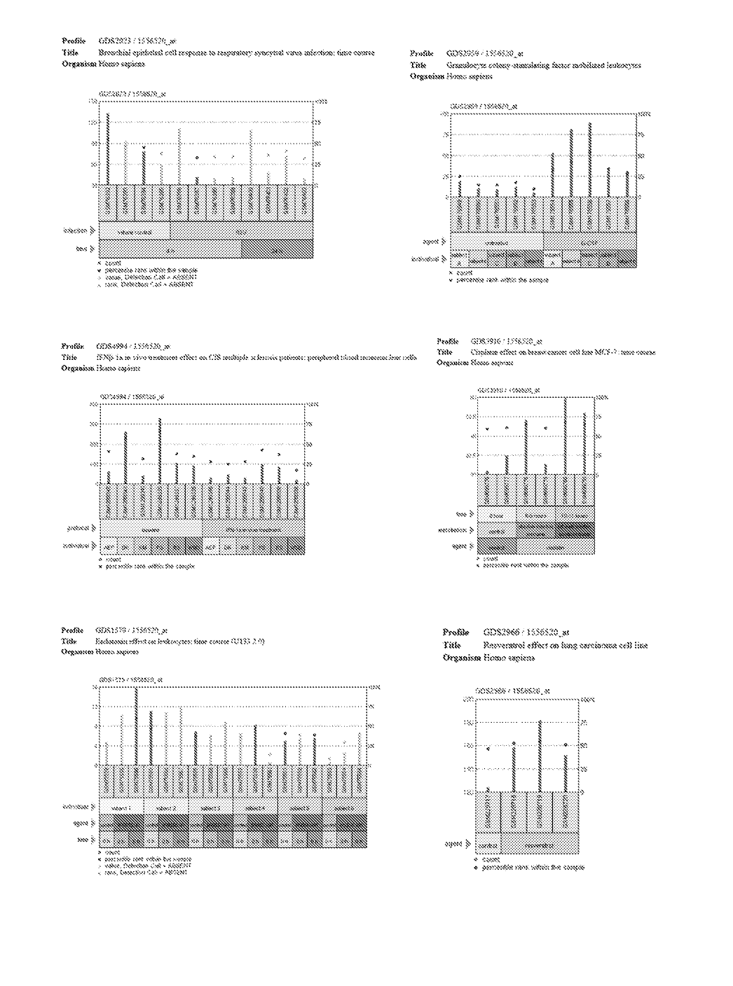

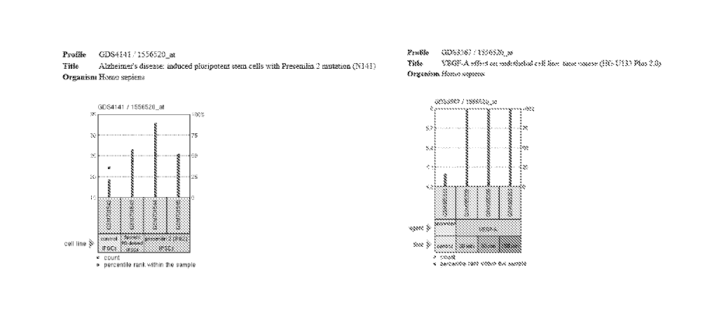

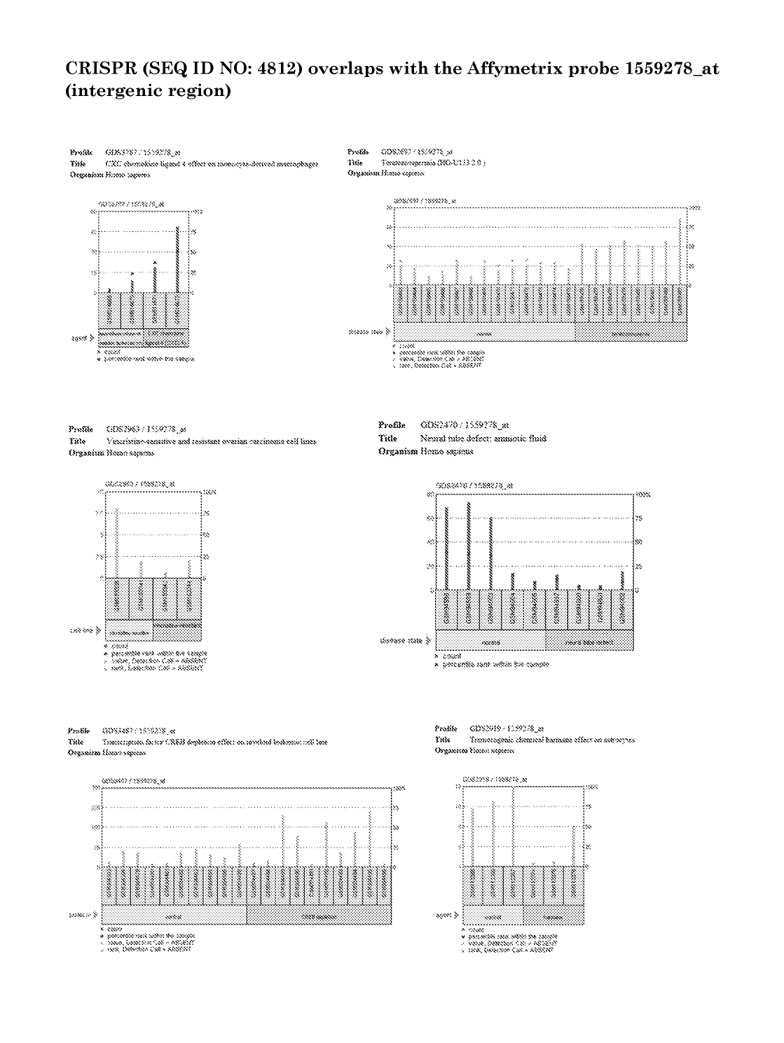

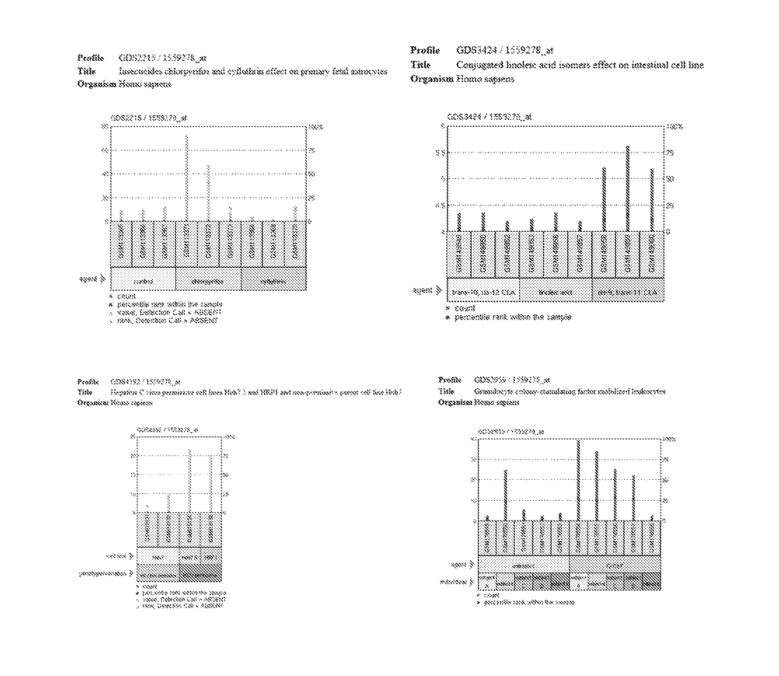

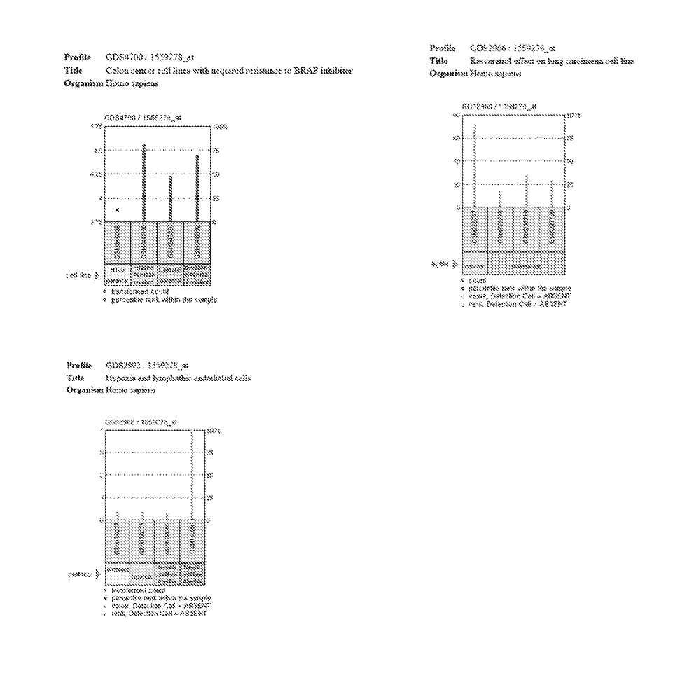

It was further established by the present inventor that these eukaryotic CRISPR sequences were transcribed, since RNA transcripts derived from the CRISPR sequences have been shown to be present in eukaryotic cells (see FIG. 2A-F and Table 1). Accordingly, these CRISPRs are controlled by an endogenous promoter.

The role of these CRISPRs is yet unknown, but the present invention provides means for using the eukaryotic CRISPR sequences in assays to study their role and the interaction with gene regulation. Further, the assays described herein will enable studying the factors that are able to activate the eukaryotic CRISPRs and the interaction of the eukaryotic CRISPRs with the prokaryotic CRISPR-Cas systems that are used for genetic editing of eukaryotic cells.

In one of the assays of the invention a eukaryotic CRISPR sequence, e.g. selected from one of the Tables IA-IX and Tables IIA-IIM of the priority document PCT/NL2015/050438, with some additions now presented as SEQ ID NO: 1-11297, is either put under control of its own endogenous promoter sequences or under control of a eukaryotic promoter. It will of course depend on the nature and characteristics of the host cells which are transfected with such a vector which promoters would be suitable for driving expression of the CRISPR sequence(s). If the host cells are from the same organism as the CRISPR that is to be assayed, it would be possible to use the endogenous promoter. With respect to heterologous promoters, for plant cells plant specific promoters such as the CaMV 35S promoter or the Rubisco promoter may be taken, for insect cells a baculovirus promoter and for mammalian cells e.g. the SV40 promoter, CMV promoter or the EF-1 promoter may be used. Other promoters that may equally well be used are known to the skilled person. Alternatively, a (commercial) expression vector may be used, like the mammalian multi-purpose Flexi® Vectors, the pCMVTNT™ Vector, the pTargeT™ T-Vector, Regulated Mammalian Expression Systems, and the CheckMate™ Systems (all obtainable form Promega), adapted pCAGGS, pSC101 and other recombinase vectors (obtainable from Gene Bridges, Germany) or several plant expression vectors (see e.g. Tzfira, T. et al., 2007, Plant Physiol. 145:1087-1089). A heterologous promoter as indicated above is defined herein as a promoter that in nature does not drive expression of the CRISPR sequence with which it is connected in the vector, while an endogenous promoter is defined as the promoter that in nature does drive expression of the CRISPR that is present in the vector.

It is demonstrated that expression of most of the eukaryotic CRISPRs, especially those that have spacers which are directed to endogenous nucleotide sequences, may lead to changes in the gene regulation of the cell in which it is expressed. This is likely to be caused by the same mechanism that is also used with the CRISPR technology in prokaryotes: through RNAi. As has been mentioned above, the CRISPR sequences are expressed in the eukaryotic cell, but they are expressed in small, siRNA-like sequences. The fact that RNAi also may be applied in the eukaryotic cell has been established long ago (the scientific work was found fit for the Nobel prize), and RNAi has since then been one of the mechanisms to influence gene expression (predominantly used in genetic engineering of plant cells and for studying gene expression and gene knock-out). In the experimental part it has been established that indeed expression of the CRISPR sequences would lead to a change in the regulation of the expression of a genetic sequence by inhibiting the target to which the CRISPR sequence is directed. The effect on the expression depends on the nature of the element that is inhibited: if the element is (part of) the coding sequence or an enhancer element of such a coding sequence the expression of the product is generally inhibited. If the CRISPR is directed to an element that normally inhibits expression, the inhibition will be lifted as a result of the CRISPR sequence and expression will be enhanced.

Whether the enzyme DICER is involved in the processing of the CRISPR sequences into the RNAi-like mRNA sequences or whether an endogenous Cas gene or any other potential nuclease is responsible for this, is currently unknown. It has been shown in the experimental part that introduction of a (bacterial) Cas9 enzyme without further introduction of any guide RNA causes a plurality of doubles-stranded breaks in the DNA of an eukaryotic cell. It has further been shown that these double-stranded breaks occur at places that are considered to be targets for one (or more) of the eukaryotic CRISPR sequences of the present application. The conclusion thus is that a bacterial Cas9 enzyme is capable of mobilizing eukaryotic CRISPR sequences and use these as guide to the target sequence in order to perform the enzymatic function and to cause a double-stranded break in the target DNA. Further enhancement of this effect can be achieved by introducing, next to the Cas9 enzyme (or an enzyme that is functionally equivalent with Cas9, such as Cpf1) a vector harboring an eukaryotic CRISPR as presented in the present invention. This will cause an overexpression of the eukaryotic CRISPR sequence and thus it will increase the interaction between the enzyme and the CRISPR and thereby the effect of the CRISPR.

Thus, in general, whether or not in the presence of an endogenous or exogenous (heterologous) Cas enzyme, the production of the RNAi-like transcripts effects changes in the expression profile of the cell. Such changes may be measured in the assay according to the present invention by measuring the total transcriptome (i.e. the total amount and nature of the RNA produced) of the cell. By measuring the transcriptome of the cell for a specific CRISPR sequence and compare this to the transcriptome of a similar cell without expression of said specific CRISPR sequence, one is capable to determine the effects of the RNAi products that are produced by the expression of said eukaryotic CRISPR sequence on the expression of the gene or genes that are targeted, either directly or through any expression regulation sequence (such as enhancers or inhibitory sequences).

Methods to determine the transcriptome of a cell are sufficiently known to the skilled person. One such a method is known as RNA-seq (or Whole Transcriptome Shotgun Sequencing (WTSS)), which involves isolation of the RNA that is produced in the cell and sequencing this RNA with the use of deep sequencing technology (such as provided by Illumines,ChIP-seq or 454 Life Sciences).

Another type of assay is intended to study the mechanisms that cause expression of the CRISPR. It is shown in the experimental part that expression of the CRISPR sequences is dependent on factors, such as cell type, organ, etc. Although the precise function of this specific expression of the eukaryotic CRISPR sequences is still unknown, it may be that the expression is dependent on (or may be causing) development processes, such as cell growth and differentiation or any other endogenous or exogenous factor that may influence the (genetic) regulatory behavior of the cell.

For this assay a vector is used where the eukaryotic CRISPR sequence is under control of its endogenous promoter and the cell in which the vector has been introduced will be subjected to a stimulus, after which stimulus the transcriptome of the cell is studied for the occurrence of RNA sequences that are transcribed from the CRISPR under study. The stimulus can he a physical stimulus, such as temperature or pH, but alternatively a chemical stimulus may be administered. Such a chemical stimulus can be the administration of an endogenous compound, such as a hormone, a cytokine, a nucleotide sequence or an enzyme. It may also be a compound that does not naturally occur in the cell in which the vector has been introduced, such as an RNAi construct, a nuclease, or a Cas enzyme. When using exogenous stimuli, such as compounds that are typically used for CRISPR-Cas9 gene editing, the off-targeting that often occurs when engineering eukaryotic cells can easily be studied. A further variation on this assay can be made when such an off-targeting effect is found to use the assay to find compounds that may inhibit off-targeting. In this case a cell is provided with a vector having a eukaryotic CRISPR sequence according to the invention and a compound that is tested for its inhibition of off-targeting. Then, the cell is stimulated with the stimulus of which it is known that it causes transcription of the CRISPR and its corresponding effects on endogenous gene expression. In this way an inhibitor of off-targeting effect may be found. Of course then a final test may be to test the effect of a CRISPR-Cas gene-editing cassette in a eukaryotic target cell (which is known to have the eukaryotic CRISPR of which the off-targeting will be inhibited) in the presence of said inhibitor and to see whether now indeed the intended editing of the gene with the CRISPR-Cas system has taken place. Of course, next to Cas9 any enzyme that is capable of exerting double-stranded breaks and being targeted through an RNA guide, such as Cas9 variants and enzymes like Cpf1, may be used in this respect.

Further, it is shown that various CRISPR sequences may be expressed as a result of application of a variety of chemical compounds (with or without any pharmaceutical action) or in the occurrence of a certain condition. As such, the assay in which the CRISPR is expressed may be used as an assay to find compounds that would interfere with the compound(s) or condition(s) that would cause the expression of the CRISPR. In such a way the assay may be used to find compounds that may ameliorate or inhibit a condition by affecting the expression of a CRISPR sequence that is associated with said condition.

Concomitant with the results provided in the experimental section, preferred embodiments of the invention are vectors and assays as defined herein harboring or using the sequences that have been found to have special characteristics. The SEQ ID NOs of these sequences can be found in any of Tables 1-7 or in any of FIGS. 1-7.

### EXAMPLES

### Example 1

### List of Potential Human CRISPR Gene Targets

To identify potential targets of the identified human CRISPRs as submitted in the present application the package of https:/tbioconductor.org/packages/release/bioc/html/BiocGenerics.html was down-loaded for high-through put genomic analysis. By making use of this package the human CRISPRs could be blasted efficiently after which the CRISPR blast results were combined with the NCBI NT database with gene identifier (GI) and taxonomy (tax) information. Next to that this software tool also allowed us to analyse which sites in the human genome show homology to other organisms. The taxonomic annotation of these GI numbers was obtained from the NCBI via their eutils packages. BLAST output file was loaded and extended with the original region and GI information. A total of 5977649 BLAST results with 1175610 unique hits for 31970 unique queries was loaded. This default output of BLAST does not add vital information such as the query length therefore a FastA file was loaded to annotate the BLAST results. BLAST alignments were found for 97.5736% of the queries, but 795 hits were left annotated. As the BLAST results table was excessively large (5977649), a basic filtering on query coverage was performed. Alignments that cover less than 95% of the query were spurious and could thus be safely filtered out. This filtering resulted in more than 1643613 hits to be inspected in which only 4501 Queries harbored a single BLAST hit. For 1640646 BLAST results we could retrieve taxonomy Ids and further we found hits to 3917 taxonomic divisions. Most of the BLAST alignments pointed to eukaryote targets (FIG. 1) of which more than 35.000 were homo sapiens related. Interestingly, with respect to the known CRISPR defense function for the questionable human CRISPRs only 2.01% of the spacers aligned to bacterium or viral related nucleotides, whereas bacterial and viral targets occurred more often in confirmed CRISPRs 16.98%.

### Example 2

### Human CRISPRs are Expressed in Different Cell Lines

Since the submitted human CRISPRs as provided in the present application in potential target more than 35.000 genes it means that the mean target of each human CRISPR is three human endogenous genes. In prokaryotes the CRISPRs are shown to be involved in endogenous gene regulation which could also be true for the human CRISPRs. To test whether this is true we first need to know whether the human CRISPRs are actively transcribed. Therefore CHIP-seq data obtained from control samples of the human cell lines U2OS, Caco-2, and K562 were obtained from ENCODE. The exact condition of cell line culturing, ChIP-seq analyses, sequence mapping can be found under the accession numbers; 1) GSM935288 for the U2OS cell line; 2) GSM945236 for the Caco-2 cell line and 3) SAMN04284550 for the K562 cell line at http://www.ncbi.nlm.nih.gov/biosample/. The BAM files of the ChIP-seq data were uploaded into the Integrative Genomics Viewer. The ChIP-seq data of each cell line was mapped against the Hg19 genome. The human CRISPRs provided in the present application were position based identified on the Hg19 genome and transformed in galaxy (https://bioinf-galazian.erasmusmc.nl/galaxy/) into a block definition (BED) file and imported as a red region at the exact positions were they reside in the human genome. Uploading a BED file with every known small non-coding RNA helped us to establish that the transcription of human CRISPRs was specific. Cell line identified ChIP-seq regions were visualized in grey block (arrow-like to show the transcription orientation).

FIG. 2A-F shows examples of cell line specific ChIP-seq transcripts that match with the human CRISPRs and a large number are reverse complementary orientated on the transcripts of the human genes, strongly suggestive that they fulfill a regulatory role in endogenous gene regulation as was revealed earlier for other small regulatory RNAs. Table 1 shows an overview of the tissue and cell line specific CRISPR expression data of many of the sequences contained in the sequence listing.

Lengthy table referenced here
US20180187190A1-20180705-T00001
Please refer to the end of the specification for access instructions.

### Example 3

### Human CRISPRs are Organ Specifically Transcribed

Since the human CRISPRs were found to be actively transcribed in human cell lines it was further investigated whether the human CRISPRs are tissue specifically transcribed. Therefore RNA-sequence data from the body map 2.0 dataset of different tissues of Brain, Colon, Heart, Kidney, Lung, Liver, Thyroid, White Blood cell, Skeletal muscle, Adrenal, Lymph node, Ovary, Testes, Adipose, Breast, Prostate was uploaded into the Integrative Genomics Viewer from the broad institute (https://www.broadinstitute.org/igv/). Transcripts were mapped against the Hg19 reference genome. The human CRISPRs of the present application were position based identified on the Hg19 genome and transformed in galaxy (https://bioinf-galaxian.erasmusmc.nl/galaxy/) into a block definition (BED) file and imported as a red region at the exact positions were they reside in the human genome. Tissue specific transcripts were visualized in blue, specific details on how the RNA was isolated and transcript were mapped against the human genome can be retrieved from http://www.ncbi.nlm.nih.gov/geo/query/acc.egi?ace=GSE30611. It was found that the human CRISPRs matched with specific transcripts. This means that transcription of these specific CRISPR sequences is organ-specific. FIG. 3A-E shows examples of tissue specific transcripts that match with the human CRISPRs.

### Example 4

### Construction of a Vector with a Eukaryotic CRISPR

In order to unambiguously establish that human CRISPRs are responsible for gene regulation in an RNA interference manner, RNAi expression constructs were generated synthetically by Baseclear (Leiden, The Netherlands) using the sequences from a U6 promoter, one of the human CRISPR sequences as provided herein, a termination signal and a cloning vector PUC57. Twelve examples comprising the sequences of the U6 promoter, a human CRISPR sequences that resides in the genes ADAM10, ADAMTS9-AS2, ADAM17, TUBD and IL-10 are shown*. After synthesis the generated constructs were sequenced to confirm their correctness. A final concentration of four microgram of each plasmid was lyophilized in 40 microliter of 10mM Tris buffer (PH 8,5) to a final concentration of 100 nanogram per microliter per construct. Plasmids were transformed to Escherichia coli TOP10 cells and purified using the GeneJET plasmid Miniprep kit (Thermofisher Scientific, Breda, The Netherlands) with a final concentration of 1 microgram per microliter. Plasmid were stored at minus 20 degrees Celsius until further usage. Plasmid maps are visualized in FIG. 4 and named pLOHA+SEQ ID NO that corresponds to the human CRISPR sequence * as cloned into the RNAi expression vector PUC57 and as submitted in P108037PC00.

ADAM10_plus:
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACCTTAGAAAGTAATAAT
TTCTTGGGTAGTTTGCAGTTTTAAAATTATGTTTTAAAATGGACTATCAT
ATGCTTACCGTAACTTGAAAGTATTTCGATTTCTTGGCTTTATATATCTT
GTGGAAAGGACGAAACACCAATCTTTGGTAAGTCACTTAAACACTTAGTT
TCATCCTACAAAATGTTACAGAACAGGTATCAAATCTTTGGTAAGTCACT
TAAACACTTAGTTTCATCTTTTTT; (contains human CRISPR
SEQ ID NO: 7710)
ADAM10_Minus:
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCGATGAAACTAAGTGTTTAAGTGACTTACCAAA
GATTTGATACCTGTTCTGTAACATTTTGTAGGATGAAACTAAGTGTTTAA
GTGACTTACCAAAGATTTTTTTT (contains human CRISPR SEQ
ID NO: 7710)
ADAMTS9-AS2_27_plus:
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCACAGGAAGCATGGCTCAAGGAGATTTAGAGGC
TTAATTGGCTCATGGTTCTGCAGGAAGCACAGTTGTACAGGAAGCATGGC
TCAAGGAGATTTTTTTTT (contains human CRISPR SEQ ID
NO: 1762)
ADAMTS9-AS2_27_Minus:
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCAAATCTCCTTGAGCCATGCTTCCTGTACAACT
GTGCTTCCTGCAGAACCATGAGCCAATTAAGCCTCTAAATCTCCTTGAGC
CATGCTTCCTGTTTTTTT (contains human CRISPR SEQ ID
NO: 1762)
ADAMTS9-AS2_71_plus:
TGTACAAAAAAGGAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCAATTAACTAAAGACATCGCTAGCTAATAGTGA
GGCTTCCCACCTATAATCTAAGCATAATATGTTAATTTAGCTAATGCAAT
TAACTAAAGACATCGCTAGCTAATATTTTTT (contains human
CRISPR SEQ ID NO: 1763)
ADAMTS9-AS2_71_Minus:
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCTATTAGCTAGCGATGTCTTTAGTTAATTGCAT
TAGCTAAATTAACATATTATGCTTAGATTATAGGTGGGAAGCCTCACTAT
TAGCTAGCGATGTCTTTAGTTAATTTTTTTT (contains human
CRISPR SEQ ID NO: 1763)
ADAM17_plus:
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCCAGCTAATTTTTGTATTTTTGGTAGCTGGGAT
TACAGGCATGAGCCACCACGTCCAGCTAATTTTTGTATTTTTAGTAGTTT
TTT (contains human CRISPR SEQ ID NO: 8306)
ADAM17_Minus:
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCCTACTAAAAATACAAAAATTAGCTGGACGTGG
TGGCTCATGCCTGTAATCCCAGCTACCAAAAATACAAAAATTAGCTGTTT
TTT (contains human CRISPR SEQ ID NO: 8306)
TUBD_plus:
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGACTATCATAT
GCTTACCGTAACTTGAAAGTATTTCGATTTCTTGGCTTTATATATCTTGT
GGAAAGGACGAAACACCGTCTCTACTAAAAATACAAAAATTACCATCCTG
GCTAACACGGTGAAACCCCGTCTCTACTAAAAATACAAAAATTTTTT
(contains human CRISPR SEQ ID NO: 1138)
TUBD_Minus:
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCTTTTTGTATTTTTAGTAGAGACGGGGTTTCAC
CGTGTTAGCCAGGATGGTAATTTTTGTATTTTTAGTAGAGACTTTTTT
(contains human CRISPR SEQ ID NO: 1138)
IL-10_plus:
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCGAAACCCCGTCTCTACTAAAAAAATACAAAAA
AAATTAGCCGGGCATGGTCACACAATGAAACCCCGTCTCTACTAAAAATT
TTTT (contains human CRISPR SEQ ID NO: 8156)
IL-10_Minus:
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCTTTTTAGTAGAGACGGGGTTTCATTGTGTGAC
CATGCCCGGCTAATTTTTTTTGTATTTTTTTAGTAGAGACGGGGTTTCTT
TTTT (contains human CRISPR SEQ ID NO: 8156)

### Example 5

### RNAi Assay

After synthetically generating the constructs required to validate whether the human CRISPRs are able to actively regulated endogenous gene expression an RNAi assay was developed. For this assay U2OS bone marrow epithelial cells and SKBR3 breast cancer cells were maintained in Dulbecco's modified Eagle's medium (DMEM) (Invitrogen, Breda, The Netherlands) supplemented with 10% fetal bovine serum (FBS) (Invitrogen, Breda, The Netherlands), 100 U/ml penicillin, 100 μg/ml streptomycin and 1% nonessential amino acids (NEAR) (Invitrogen, Breda, The Netherlands). U2OS and SKBR3 cells were used because they have low amounts of ADAMTS9 and in case of U2OS cells high amounts of ADAM10. The cells were cultured in a 75-cm² flask (Greiner Bio-one, Alphen aan den Rijn, The Netherlands) at 37° C. and 5% CO₂ in a humidified air incubator. For the RNAi assay U2OS and SKBR3 cells were grown to 40% to 50% confluence on chamber slides (Greiner Bio-one, Alphen aan den Rijn, The Netherlands). Cells (U2OS and SKBR3) were transiently transfected with plasmid DNA pLOHA_7710_−; pLOHA_7710_+; pLOHA_1762_− and pLOHA_1762_+ (see FIG. 4) to silence ADAM10 and ADAMTS9-AS2 expression using X-tremeGENE HP DNA Transfection Reagent (Roche Applied Science, Almere, The Netherlands), according to the manufacturer's protocols. After 24-48 hours the U2OS and SKBR3 cells were washed three times with room temperature HBSS and fixated with 4% paraformaldehyde and then permeabilized with 0.1% HBSS-Triton X-100 solution for 20 min. Background antibody binding was blocked with block buffer (1% fetal bovine serum, 1% Tween20, HBSS). Slides were then incubated for one hour with the respective primary antibody for ADAM10 or ADAMTS9 (Abeam, Cambridge, United Kingdom) at a 1:1000 dilution in block buffer. The appropriate secondary antibodies from the IgG class (H+L), A594 labeled (Molecular Probes, Bleiswijk, The Netherlands), providing a red stain were used to detect ADAM10 and ADAMTS9 thereby revealing that pLOHA_7710_+ was able to affect silencing of the expression of ADAM10 in U2OS cells (FIG. 5A). For pLOHA_1762_− it was revealed that ADAMTS9 expression was induced in both U2OS (FIG. 5B) and SKBR3 (FIG. 5C) cells, because the antisense RNA (ADAMTS9-AS2) that controls the expression of ADAMTS9 is silenced by pLOHA_1762_−. Untreated U2OS and SKBR3 cells were used as a negative control and pCDNA3.1 as an empty plasmid control (FIG. 5A-C). To confirm that pLOHA_7710_+ was able to affect the expression of ADAM10 in U2OS cells, an SDS-PAGE was performed after which the ADAM10 protein was quantified by western immunoblotting. Briefly, U2OS cells were transfected with pLOHA_7710_+, pLOHA_7710_−, pCDNA3.1 or left untreated and after 24-48 hours the four samples were homogenized in Laemmli buffer. The cell lysate was resolved on a 12% SDS-polyacrylamide gel and electroblotted to a nitrocellulose membrane (Protran; Schleicher & Schuell, Dassel, Germany). The membrane was then pre-incubated in blocking buffer (5% non-fat milk powder, 0.1% (w/v) Tween 20 in PBS) and incubated with a 1:1000 dilution of a polyclonal antibody that is specific for ADAM10 (ABCAM). Subsequently, the membrane was incubated with a 1:1000 diluted AP-conjugated appropriate secondary antibody (Promega, Leiden, The Netherlands). NBT-BCIP solution was used that reacts with the AP-conjugated appropriate secondary antibody to visualize ADAM10 expression (FIG. 5D).

From these experiments it follows that constructs with human CRISPR sequences can influence the expression of genes, both positively (enhancing expression) or negatively (inhibition of expression) depending on the function of the target sequence.

### Example 6

### Human CRISPRs Function as a Guide RNA for Cas9 to Induce Toxic Double Stranded DNA Breaks in Eukaryotic Cells

With respect to CRISPR-Cas9 genome editing the role of human CRISPRs in off targeting was also explored by making use of a specific technique called BLESS. For BLESS analysis the detailed protocol of Crosetto et al. (Nature Methods 10:361-365, 2013) was used. U2OS cells were infected for 6 hours with C. jejuni wild type, Acas9 and Acas9A and then fixated according to the Crossetto protocol and further processed for PCR and sequencing. Or, U2OS cells were transfected with pCDNA3.1+CjCas9 using HP X-tremegene transfection agent (Roche Applied Science, Almere, The Netherlands), radiated with 1 Gy or untreated and after 24 fixated according to the Crossetto protocol and further processed for PCR and sequencing. As a positive control U2OS cells with a stable integrated I-SceI site were transfected with a plasmid expressing the I-SceI enzyme harboring a nuclear localization site. After 24 hours cells were fixated according to the Crosetto protocol and further processed for PCR and sequencing (the U2OS-I-SceI cell line and plasmid containing the I-SceI-nls enzyme were kindly provided by Prof. Dik van Gent (Erasmus MC)). Analysis was performed according the Crosetto protocol with the addition that the obtained sequences were also mapped against the human CRISPR regions. In brief, we analyzed Illumina data using the Galaxy software from the bioinformatic department (Erasmus MC). We used a generated pipeline (https://bioinf-galaxian.erasmusmc.nl/galaxy/workflow) in which sequences of strand 1 and sequences of strand 2 were uploaded into the galaxian server separately and simultaneously. After upload the sequences were quality controlled using FastQC, concatenated and mapped with BWA-MEM against the hg19 genome and analyzed on the number of breaks induced, and positions of the breaks. Further, it was determined whether the breaks were PAM motif dependent or related to the human CRISPR transcripts identified in the U2OS cells. BAM files of the BLESS samples were uploaded into the Integrative Genomics Viewer. The human CRISPRs of the present application were position based identified on the Hg19 genome and transformed in galaxy (https://bioinf-galaxian.erasmusmc.nl/galaxy/) into a block definition (BED) file and imported as a red region at the exact positions where they reside in the human genome. FIG. 6A-E display examples of C. jejuni Cas9 breaks that could be complemented during the infection of U2OS cells and after transfection of CjCas9 in an eukaryotic expression vector of the same strain used for infection. Further inspection of the break sites revealed that the human CRISPRs functioned as a guide RNA. It was found that 23bp sequences of actively transcribed human CRISPRs in the U2OS cells base paired exactly in an ungapped manner with Cas9 dependent DSB break position in the genome of these eukaryotic cells during bacterial infection. This could be confirmed with plasmid transfection of Cas9 (Table 2).

Accordingly, this experiment shows that the human CRISPRs can function as a guide RNA whenever an appropriate Cas9 or Cas9-like enzyme is available.

TABLE 2
human CRISPR and CjCas9 induced toxic DSB's in U2OS cells.

| | | | No. | |
|---|---|---|---|---|
| Patent SEQ ID + hCRISPR | | Match | of | |
| position^{A} | CjCas9 induced breaksite in the genome U2OS cells^{B} | in %^{C} | bp^{D} | E-Value^{E} |
| 1109_Chr2_85737752_85738008 | Seq_GeCjCas9.OGB.20plus_chr9_66469976_66469999 | 100 | 22 | 2.00E−07 |
| 1109_Chr2_85737752_85738008 | Seq_GeCjCas9.OGB.20plus_chr9_66469976_66469999 | 100 | 22 | 2.00E−07 |
| 1138_Chr2_106855597_106856189 | Seq_GeCjCas9.OGB.20plus_chr3_134188505_134188528 | 100 | 23 | 1.00E−07 |
| 115_Chr1_24079090_24079202 | Seq_GeCjCas9.OGB.20plus_chr5_57183007_57183030 | 95.65 | 23 | 5.00E−06 |
| 115_Chr1_24079090_24079202 | Seq_GeCjCas9.OGB.20plus_chr1_237600602_237600625 | 95.65 | 23 | 5.00E−06 |
| 1333_Chr2_175844339_175844446 | Seq_GeCjCas9.OGB.20plus_chr5_31469271_31469294 | 95.65 | 23 | 5.00E−06 |
| 1419_Chr2_202729300_202729405 | Seq_GeCjCas9.OGB.20plus_chr9_66469976_66469999 | 95.65 | 23 | 4.00E−06 |
| 1471_Chr2_219072954_219073050 | Seq_GeCjCas9.OGB.20plus_chr1_237600602_237600625 | 100 | 23 | 2.00E−08 |
| 1471_Chr2_219072954_219073050 | Seq_GeCjCas9.OGB.20plus_chr1_237600602_237600625 | 100 | 22 | 7.00E−08 |
| 1473_Chr2_219713963_219714078 | Seq_GeCjCas9.OGB.20plus_chr15_57336567_57336590 | 95.65 | 23 | 5.00E−06 |
| 1513_Chr2_232501114_232501223 | Seq_GeCjCas9.OGB.20plus_chr9_66469976_66469999 | 100 | 23 | 2.00E−08 |
| 1513_Chr2_232501114_232501223 | Seq_GeCjCas9.OGB.20plus_chr2_54032605_54032628 | 95.65 | 23 | 5.00E−06 |
| 1513_Chr2_232501114_232501223 | Seq_GeCjCas9.OGB.20plus_chr1_150539667_150539690 | 95.65 | 23 | 5.00E−06 |
| 1565_Chr2_242262774_242262909 | Seq_GeCjCas9.OGB.20plus_chr5_31469271_31469294 | 95.65 | 23 | 6.00E−06 |
| 1565_Chr2_242262774_242262909 | Seq_GeCjCas9.OGB.20plus_chr12_47579379_47579402 | 95.65 | 23 | 6.00E−06 |
| 1611_Chr3_14667912_14668025 | Seq_GeCjCas9.OGB.20plus_chr5_57183007_57183030 | 95.65 | 23 | 5.00E−06 |
| 1611_Chr3_14667912_14668025 | Seq_GeCjCas9.OGB.20plus_chr1_237600602_237600625 | 95.65 | 23 | 5.00E−06 |
| 1611_Chr3_14667912_14668025 | Seq_GeCjCas9.OGB.20plus_chr9_66469976_66469999 | 100 | 22 | 8.00E−08 |
| 1631_Chr3_19957215_19957290 | Seq_GeCjCas9.OGB.20plus_chr5_31469271_31469294 | 95.65 | 23 | 3.00E−06 |
| 1631_Chr3_19957215_19957290 | Seq_GeCjCas9.OGB.20plus_chr12_47579379_47579402 | 95.65 | 23 | 3.00E−06 |
| 1672_Chr3_32692995_32693124 | Seq_GeCjCas9.OGB.20plus_chr9_66469976_66469999 | 100 | 23 | 2.00E−08 |
| 1672_Chr3_32692995_32693124 | Seq_GeCjCas9.OGB.20plus_chr2_54032605_54032628 | 95.65 | 23 | 6.00E−06 |
| 1672_Chr3_32692995_32693124 | Seq_GeCjCas9.OGB.20plus_chr1_150539667_150539690 | 95.65 | 23 | 6.00E−06 |
| 1672_Chr3_32692995_32693124 | Seq_GeCjCas9.OGB.20plus_chr9_66469976_66469999 | 100 | 22 | 9.00E−08 |
| 183_Chr1_43651312_43651417 | Seq_GeCjCas9.OGB.20plus_chr11_77993558_77993581 | 95.65 | 23 | 4.00E−06 |
| 1964_Chr3_136424743_136424831 | Seq_GeCjCas9.OGB.20plus_chr2_84646045_84646068 | 95.65 | 23 | 4.00E−06 |
| 1964_Chr3_136424743_136424831 | Seq_GeCjCas9.OGB.20plus_chr2_84646045_84646068 | 95.65 | 23 | 4.00E−06 |
| 2131_Chr3_185312627_185312778 | Seq_GeCjCas9.OGB.20plus_chr22_20325979_20326002 | 95.65 | 23 | 7.00E−06 |
| 2227_Chr4_1708589_1708850 | Seq_GeCjCas9.OGB.20plus_chr9_66469976_66469999 | 100 | 22 | 2.00E−07 |
| 2227_Chr4_1708589_1708850 | Seq_GeCjCas9.OGB.20plus_chr9_66469976_66469999 | 100 | 22 | 2.00E−07 |
| 2231_Chr4_3335028_3335150 | Seq_GeCjCas9.OGB.20plus_chr9_66469976_66469999 | 95.65 | 23 | 5.00E−06 |
| 2602_Chr4_111209282_111209397 | Seq_GeCjCas9.OGB.20plus_chr1_150539667_150539690 | 100 | 23 | 2.00E−08 |
| 2602_Chr4_111209282_111209397 | Seq_GeCjCas9.OGB.20plus_chr9_66469976_66469999 | 95.65 | 23 | 5.00E−06 |
| 2602_Chr4_111209282_111209397 | Seq_GeCjCas9.OGB.20plus_chr14_105785533_105785556 | 95.65 | 23 | 5.00E−06 |
| 2602_Chr4_111209282_111209397 | Seq_GeCjCas9.OGB.20plus_chr5_57183007_57183030 | 100 | 22 | 8.00E−08 |
| 2713_Chr4_148579989_148580100 | Seq_GeCjCas9.OGB.20plus_chr9_66469976_66469999 | 100 | 23 | 2.00E−08 |
| 2713_Chr4_148579989_148580100 | Seq_GeCjCas9.OGB.20plus_chr9_66469976_66469999 | 95.65 | 23 | 5.00E−06 |
| 2713_Chr4_148579989_148580100 | Seq_GeCjCas9.OGB.20plus_chr2_54032605_54032628 | 95.65 | 23 | 5.00E−06 |
| 2713_Chr4_148579989_148580100 | Seq_GeCjCas9.OGB.20plus_chr1_150539667_150539690 | 95.65 | 23 | 5.00E−06 |
| 2713_Chr4_148579989_148580100 | Seq_GeCjCas9.OGB.20plus_chr1_237600602_237600625 | 100 | 22 | 8.00E−08 |
| 2765_Chr4_165781005_165781116 | Seq_GeCjCas9.OGB.20plus_chr17_4861121_4861144 | 95.65 | 23 | 5.00E−06 |
| 2765_Chr4_165781005_165781116 | Seq_GeCjCas9.OGB.20plus_chr5_57183007_57183030 | 95.65 | 23 | 5.00E−06 |
| 2765_Chr4_165781005_165781116 | Seq_GeCjCas9.OGB.20plus_chr1_237600602_237600625 | 95.65 | 23 | 5.00E−06 |
| 2765_Chr4_165781005_165781116 | Seq_GeCjCas9.OGB.20plus_chr9_66469976_66469999 | 100 | 22 | 8.00E−08 |
| 3081_Chr5_37367004_37367113 | Seq_GeCjCas9.OGB.20plus_chr9_66469976_66469999 | 100 | 23 | 2.00E−08 |
| 3081_Chr5_37367004_37367113 | Seq_GeCjCas9.OGB.20plus_chr2_54032605_54032628 | 95.65 | 23 | 5.00E−06 |
| 3081_Chr5_37367004_37367113 | Seq_GeCjCas9.OGB.20plus_chr1_150539667_150539690 | 95.65 | 23 | 5.00E−06 |
| 3095_Chr5_40817519_40817633 | Seq_GeCjCas9.OGB.20plus_chr9_66469976_66469999 | 100 | 23 | 2.00E−08 |
| 3095_Chr5_40817519_40817633 | Seq_GeCjCas9.OGB.20plus_chr9_66469976_66469999 | 95.65 | 23 | 5.00E−06 |
| 3095_Chr5_40817519_40817633 | Seq_GeCjCas9.OGB.20plus_chr2_54032605_54032628 | 95.65 | 23 | 5.00E−06 |
| 3095_Chr5_40817519_40817633 | Seq_GeCjCas9.OGB.20plus_chr1_150539667_150539690 | 95.65 | 23 | 5.00E−06 |
| 3595_Chr6_28955550_28955804 | Seq_GeCjCas9.OGB.20plus_chr17_4861121_4861144 | 100 | 23 | 4.00E−08 |
| 3595_Chr6_28955550_28955804 | Seq_GeCjCas9.OGB.20plus_chr9_66469976_66469999 | 100 | 22 | 2.00E−07 |
| 3660_Chr6_47470473_47470577 | Seq_GeCjCas9.OGB.20plus_chr9_66469976_66469999 | 95.65 | 23 | 4.00E−06 |
| 3696_Chr6_58244470_58244582 | Seq_GeCjCas9.OGB.20plus_chr5_57183007_57183030 | 95.65 | 23 | 5.00E−06 |
| 3696_Chr6_58244470_58244582 | Seq_GeCjCas9.OGB.20plus_chr5_57183007_57183030 | 95.65 | 23 | 5.00E−06 |
| 3696_Chr6_58244470_58244582 | Seq_GeCjCas9.OGB.20plus_chr1_237600602_237600625 | 95.65 | 23 | 5.00E−06 |
| 3696_Chr6_58244470_58244582 | Seq_GeCjCas9.OGB.20plus_chr1_237600602_237600625 | 95.65 | 23 | 5.00E−06 |
| 3696_Chr6_58244470_58244582 | Seq_GeCjCas9.OGB.20plus_chr9_66469976_66469999 | 100 | 22 | 8.00E−08 |
| 3696_Chr6_58244470_58244582 | Seq_GeCjCas9.OGB.20plus_chr9_66469976_66469999 | 100 | 22 | 8.00E−08 |
| 3716_Chr6_64381823_64381962 | Seq_GeCjCas9.OGB.20plus_chr5_31469271_31469294 | 95.65 | 23 | 6.00E−06 |
| 3716_Chr6_64381823_64381962 | Seq_GeCjCas9.OGB.20plus_chr12_47579379_47579402 | 95.65 | 23 | 6.00E−06 |
| 4012_Chr6_158992376_158992468 | Seq_GeCjCas9.OGB.20plus_chr9_66469976_66469999 | 100 | 23 | 2.00E−08 |
| 4012_Chr6_158992376_158992468 | Seq_GeCjCas9.OGB.20plus_chr9_66469976_66469999 | 95.65 | 23 | 4.00E−06 |
| 4012_Chr6_158992376_158992468 | Seq_GeCjCas9.OGB.20plus_chr2_54032605_54032628 | 95.65 | 23 | 4.00E−06 |
| 4012_Chr6_158992376_158992468 | Seq_GeCjCas9.OGB.20plus_chr1_150539667_150539690 | 95.65 | 23 | 4.00E−06 |
| 4120_Chr7_5207255_5207379 | Seq_GeCjCas9.OGB.20plus_chr5_31469271_31469294 | 95.65 | 23 | 5.00E−06 |
| 4120_Chr7_5207255_5207379 | Seq_GeCjCas9.OGB.20plus_chr12_47579379_47579402 | 95.65 | 23 | 5.00E−06 |
| 4122_Chr7_5841079_5841203 | Seq_GeCjCas9.OGB.20plus_chr5_31469271_31469294 | 95.65 | 23 | 5.00E−06 |
| 4122_Chr7_5841079_5841203 | Seq_GeCjCas9.OGB.20plus_chr12_47579379_47579402 | 95.65 | 23 | 5.00E−06 |
| 4247_Chr7_49354671_49354751 | Seq_GeCjCas9.OGB.20plus_chr5_31469271_31469294 | 95.65 | 23 | 3.00E−06 |
| 4247_Chr7_49354671_49354751 | Seq_GeCjCas9.OGB.20plus_chr12_47579379_47579402 | 95.65 | 23 | 3.00E−06 |
| 4256_Chr7_62033801_62033942 | Seq_GeCjCas9.OGB.20plus_chr2_92316200_92316223 | 100 | 23 | 3.00E−08 |
| 4256_Chr7_62033801_62033942 | Seq_GeCjCas9.OGB.20plus_chr2_92316200_92316223 | 95.65 | 23 | 6.00E−06 |
| 4256_Chr7_62033801_62033942 | Seq_GeCjCas9.OGB.20plus_chr10_42404383_42404406 | 95.65 | 23 | 6.00E−06 |
| 4256_Chr7_62033801_62033942 | Seq_GeCjCas9.OGB.20plus_chr10_42398148_42398171 | 95.65 | 23 | 6.00E−06 |
| 4258_Chr7_62986233_62986335 | Seq_GeCjCas9.OGB.20plus_chr5_57183007_57183030 | 95.65 | 23 | 4.00E−06 |
| 4258_Chr7_62986233_62986335 | Seq_GeCjCas9.OGB.20plus_chr1_237600602_237600625 | 95.65 | 23 | 4.00E−06 |
| 4258_Chr7_62986233_62986335 | Seq_GeCjCas9.OGB.20plus_chr9_66469976_66469999 | 100 | 22 | 7.00E−08 |
| 4259_Chr7_63025117_63025233 | Seq_GeCjCas9.OGB.20plus_chr2_54032605_54032628 | 95.65 | 23 | 5.00E−06 |
| 4267_Chr7_65452039_65452140 | Seq_GeCjCas9.OGB.20plus_chr9_66469976_66469999 | 95.65 | 23 | 4.00E−06 |
| 4323_Chr7_75182490_75182581 | Seq_GeCjCas9.OGB.20plus_chr5_57183007_57183030 | 95.65 | 23 | 4.00E−06 |
| 4323_Chr7_75182490_75182581 | Seq_GeCjCas9.OGB.20plus_chr1_237600602_237600625 | 95.65 | 23 | 4.00E−06 |
| 4323_Chr7_75182490_75182581 | Seq_GeCjCas9.OGB.20plus_chr9_66469976_66469999 | 100 | 22 | 6.00E−08 |
| 4331_Chr7_76985344_76985448 | Seq_GeCjCas9.OGB.20plus_chr5_57183007_57183030 | 95.65 | 23 | 4.00E−06 |
| 4331_Chr7_76985344_76985448 | Seq_GeCjCas9.OGB.20plus_chr1_237600602_237600625 | 95.65 | 23 | 4.00E−06 |
| 4331_Chr7_76985344_76985448 | Seq_GeCjCas9.OGB.20plus_chr9_66469976_66469999 | 100 | 22 | 7.00E−08 |
| 4428_Chr7_98817493_98817576 | Seq_GeCjCas9.OGB.20plus_chr5_31469271_31469294 | 95.65 | 23 | 3.00E−06 |
| 483_Chr1_161313749_161313849 | Seq_GeCjCas9.OGB.20plus_chr4_25224555_25224578 | 95.65 | 23 | 4.00E−06 |
| 4932_Chr8_66296708_66296805 | Seq_GeCjCas9.OGB.20plus_chr8_58176271_58176294 | 95.65 | 23 | 4.00E−06 |
| 4932_Chr8_66296708_66296805 | Seq_GeCjCas9.OGB.20plus_chr10_69315106_69315129 | 95.65 | 23 | 4.00E−06 |
| 5328_Chr9_39478934_39479041 | Seq_GeCjCas9.OGB.20plus_chr5_57183007_57183030 | 95.65 | 23 | 5.00E−06 |
| 5328_Chr9_39478934_39479041 | Seq_GeCjCas9.OGB.20plus_chr1_237600602_237600625 | 95.65 | 23 | 5.00E−06 |
| 5328_Chr9_39478934_39479041 | Seq_GeCjCas9.OGB.20plus_chr9_66469976_66469999 | 100 | 22 | 7.00E−08 |
| 5328_Chr9_40806509_40806616 | Seq_GeCjCas9.OGB.20plus_chr5_57183007_57183030 | 95.65 | 23 | 5.00E−06 |
| 5328_Chr9_40806509_40806616 | Seq_GeCjCas9.OGB.20plus_chr1_237600602_237600625 | 95.65 | 23 | 5.00E−06 |
| 5328_Chr9_40806509_40806616 | Seq_GeCjCas9.OGB.20plus_chr9_66469976_66469999 | 100 | 22 | 7.00E−08 |
| 5328_Chr9_41623952_41624059 | Seq_GeCjCas9.OGB.20plus_chr5_57183007_57183030 | 95.65 | 23 | 5.00E−06 |
| 5328_Chr9_41623952_41624059 | Seq_GeCjCas9.OGB.20plus_chr1_237600602_237600625 | 95.65 | 23 | 5.00E−06 |
| 5328_Chr9_41623952_41624059 | Seq_GeCjCas9.OGB.20plus_chr9_66469976_66469999 | 100 | 22 | 7.00E−08 |
| 5416_Chr9_97060762_97060846 | Seq_GeCjCas9.OGB.20plus_chr15_57336567_57336590 | 95.65 | 23 | 3.00E−06 |
| 5421_Chr9_98070799_98070929 | Seq_GeCjCas9.OGB.20plus_chr9_66469976_66469999 | 100 | 23 | 2.00E−08 |
| 5421_Chr9_98070799_98070929 | Seq_GeCjCas9.OGB.20plus_chr2_54032605_54032628 | 95.65 | 23 | 6.00E−06 |
| 5421_Chr9_98070799_98070929 | Seq_GeCjCas9.OGB.20plus_chr1_150539667_150539690 | 95.65 | 23 | 6.00E−06 |
| 5469_Chr9_115520799_115520894 | Seq_GeCjCas9.OGB.20plus_chr3_134188505_134188528 | 95.65 | 23 | 4.00E−06 |
| 5562_Chr9_139952108_139952652 | Seq_GeCjCas9.OGB.20plus_chr2_54032605_54032628 | 100 | 22 | 4.00E−07 |
| 5636_Chr10_5923903_5924012 | Seq_GeCjCas9.OGB.20plus_chr9_66469976_66469999 | 100 | 23 | 2.00E−08 |
| 5636_Chr10_5923903_5924012 | Seq_GeCjCas9.OGB.20plus_chr2_54032605_54032628 | 95.65 | 23 | 5.00E−06 |
| 5636_Chr10_5923903_5924012 | Seq_GeCjCas9.OGB.20plus_chr1_150539667_150539690 | 95.65 | 23 | 5.00E−06 |
| 5690_Chr10_25063890_25063983 | Seq_GeCjCas9.OGB.20plus_chr2_54032605_54032628 | 95.65 | 23 | 4.00E−06 |
| 5733_Chr10_35354207_35354310 | Seq_GeCjCas9.OGB.20plus_chr9_66469976_66469999 | 95.65 | 23 | 4.00E−06 |
| 5840_Chr10_70449270_70449388 | Seq_GeCjCas9.OGB.20plus_chr15_57336567_57336590 | 95.65 | 23 | 5.00E−06 |
| 5927_Chr10_99470981_99471218 | Seq_GeCjCas9.OGB.20plus_chr15_57336567_57336590 | 95.65 | 23 | 1.00E−05 |
| 5931_Chr10_100924520_100924608 | Seq_GeCjCas9.OGB.20plus_chr9_66469976_66469999 | 100 | 22 | 6.00E−08 |
| 6071_Chr11_426264_426358 | Seq_GeCjCas9.OGB.20plus_chr4_25224555_25224578 | 95.65 | 23 | 4.00E−06 |
| 6215_Chr11_45570011_45570123 | Seq_GeCjCas9.OGB.20plus_chr9_66469976_66469999 | 100 | 22 | 8.00E−08 |
| 6224_Chr11_48839300_48839406 | Seq_GeCjCas9.OGB.20plus_chr2_92305595_92305618 | 100 | 22 | 7.00E−08 |
| 6224_Chr11_48839300_48839406 | Seq_GeCjCas9.OGB.20plus_chr7_61969183_61969206 | 95.65 | 23 | 4.00E−06 |
| 6224_Chr11_48839300_48839406 | Seq_GeCjCas9.OGB.20plus_chr10_42406652_42406675 | 95.65 | 23 | 4.00E−06 |
| 6224_Chr11_48839300_48839406 | Seq_GeCjCas9.OGB.20plus_chr10_42534960_42534983 | 95.65 | 23 | 4.00E−06 |
| 6251_Chr11_62746325_62746440 | Seq_GeCjCas9.OGB.20plus_chr9_66469976_66469999 | 95.65 | 23 | 5.00E−06 |
| 6366_Chr11_97961502_97961616 | Seq_GeCjCas9.OGB.20plus_chr5_57183007_57183030 | 95.65 | 23 | 5.00E−06 |
| 6366_Chr11_97961502_97961616 | Seq_GeCjCas9.OGB.20plus_chr1_237600602_237600625 | 95.65 | 23 | 5.00E−06 |
| 6366_Chr11_97961502_97961616 | Seq_GeCjCas9.OGB.20plus_chr9_66469976_66469999 | 100 | 22 | 8.00E−08 |
| 6381_Chr11_102133291_102133396 | Seq_GeCjCas9.OGB.20plus_chr15_57336567_57336590 | 95.65 | 23 | 4.00E−06 |
| 6430_Chr11_116672942_116673037 | Seq_GeCjCas9.OGB.20plus_chr5_57183007_57183030 | 95.65 | 23 | 4.00E−06 |
| 6430_Chr11_116672942_116673037 | Seq_GeCjCas9.OGB.20plus_chr5_57183007_57183030 | 95.65 | 23 | 4.00E−06 |
| 6430_Chr11_116672942_116673037 | Seq_GeCjCas9.OGB.20plus_chr1_237600602_237600625 | 95.65 | 23 | 4.00E−06 |
| 6430_Chr11_116672942_116673037 | Seq_GeCjCas9.OGB.20plus_chr1_237600602_237600625 | 95.65 | 23 | 4.00E−06 |
| 6430_Chr11_116672942_116673037 | Seq_GeCjCas9.OGB.20plus_chr9_66469976_66469999 | 100 | 22 | 6.00E−08 |
| 6430_Chr11_116672942_116673037 | Seq_GeCjCas9.OGB.20plus_chr9_66469976_66469999 | 100 | 22 | 6.00E−08 |
| 6549_Chr12_19578356_19578461 | Seq_GeCjCas9.OGB.20plus_chr5_57183007_57183030 | 95.65 | 23 | 4.00E−06 |
| 6549_Chr12_19578356_19578461 | Seq_GeCjCas9.OGB.20plus_chr1_237600602_237600625 | 95.65 | 23 | 4.00E−06 |
| 6549_Chr12_19578356_19578461 | Seq_GeCjCas9.OGB.20plus_chr9_66469976_66469999 | 100 | 22 | 7.00E−08 |
| 6622_Chr12_34685662_34685768 | Seq_GeCjCas9.OGB.20plus_chr1_121485297_121485320 | 95.65 | 23 | 4.00E−06 |
| 67_Chr1_8320950_8321039 | Seq_GeCjCas9.OGB.20plus_chr9_66469976_66469999 | 100 | 23 | 2.00E−08 |
| 67_Chr1_8320950_8321039 | Seq_GeCjCas9.OGB.20plus_chr2_54032605_54032628 | 95.65 | 23 | 4.00E−06 |
| 67_Chr1_8320950_8321039 | Seq_GeCjCas9.OGB.20plus_chr1_160639667_160639690 | 95.65 | 23 | 4.00E−06 |
| 676_Chr1_220181116_220181196 | Seq_GeCjCas9.OGB.20plus_chr9_66469976_66469999 | 95.65 | 23 | 3.00E−06 |
| 6811_Chr12_101866663_101866762 | Seq_GeCjCas9.OGB.20plus_chr5_31469271_31469294 | 95.65 | 23 | 4.00E−06 |
| 6811_Chr12_101866663_101866762 | Seq_GeCjCas9.OGB.20plus_chr12_47579379_47579402 | 95.65 | 23 | 4.00E−06 |
| 6838_Chr12_111068960_111069049 | Seq_GeCjCas9.OGB.20plus_chr15_57336567_57336590 | 95.65 | 23 | 4.00E−06 |
| 6871_Chr12_122174321_122174450 | Seq_GeCjCas9.OGB.20plus_chr9_66469976_66469999 | 100 | 23 | 2.00E−08 |
| 6871_Chr12_122174321_122174450 | Seq_GeCjCas9.OGB.20plus_chr9_66469976_66469999 | 100 | 23 | 2.00E−08 |
| 6871_Chr12_122174321_122174450 | Seq_GeCjCas9.OGB.20plus_chr2_54032605_54032628 | 95.65 | 23 | 6.00E−06 |
| 6871_Chr12_122174321_122174450 | Seq_GeCjCas9.OGB.20plus_chr2_54032605_54032628 | 95.65 | 23 | 6.00E−06 |
| 6871_Chr12_122174321_122174450 | Seq_GeCjCas9.OGB.20plus_chr1_150539667_150539690 | 95.65 | 23 | 6.00E−06 |
| 6871_Chr12_122174321_122174450 | Seq_GeCjCas9.OGB.20plus_chr1_150539667_150539690 | 95.65 | 23 | 6.00E−06 |
| 6877_Chr12_123951337_123951461 | Seq_GeCjCas9.OGB.20plus_chr9_66469976_66469999 | 100 | 23 | 2.00E−08 |
| 6877_Chr12_123951337_123951461 | Seq_GeCjCas9.OGB.20plus_chr2_54032605_54032628 | 95.65 | 23 | 5.00E−06 |
| 6877_Chr12_123951337_123951461 | Seq_GeCjCas9.OGB.20plus_chr1_150539667_150539690 | 95.65 | 23 | 5.00E−06 |
| 6922_Chr12_132006844_132006971 | Seq_GeCjCas9.OGB.20plus_chr18_1789852_1789875 | 100 | 23 | 2.00E−08 |
| 711_Chr1_225671162_225671256 | Seq_GeCjCas9.OGB.20plus_chr11_77993558_77993581 | 95.65 | 23 | 4.00E−06 |
| 7342_Chr14_28266246_28266360 | Seq_GeCjCas9.OGB.20plus_chr9_66469976_66469999 | 100 | 22 | 8.00E−08 |
| 7368_Chr14_35165026_35165163 | Seq_GeCjCas9.OGB.20plus_chr17_4861121_4861144 | 100 | 23 | 2.00E−08 |
| 7432_Chr14_59108883_59109015 | Seq_GeCjCas9.OGB.20plus_chr2_54032605_54032628 | 100 | 23 | 2.00E−08 |
| 7432_Chr14_59108883_59109015 | Seq_GeCjCas9.OGB.20plus_chr9_66469976_66469999 | 95.65 | 23 | 6.00E−06 |
| 7542_Chr14_96886299_96886423 | Seq_GeCjCas9.OGB.20plus_chr15_57336567_57336590 | 95.65 | 23 | 5.00E−06 |
| 7542_Chr14_96886299_96886423 | Seq_GeCjCas9.OGB.20plus_chr15_57336567_57336590 | 95.65 | 23 | 5.00E−06 |
| 7565_Chr14_102517614_102517709 | Seq_GeCjCas9.OGB.20plus_chr17_4861121_4861144 | 100 | 22 | 6.00E−08 |
| 7565_Chr14_102517614_102517709 | Seq_GeCjCas9.OGB.20plus_chr5_57183007_57183030 | 95.65 | 23 | 4.00E−06 |
| 7565_Chr14_102517614_102517709 | Seq_GeCjCas9.OGB.20plus_chr1_237600602_237600625 | 95.65 | 23 | 4.00E−06 |
| 7565_Chr14_102517614_102517709 | Seq_GeCjCas9.OGB.20plus_chr9_66469976_66469999 | 100 | 22 | 6.00E−08 |
| 7645_Chr15_33901557_33901681 | Seq_GeCjCas9.OGB.20plus_chr5_57183007_57183030 | 95.65 | 23 | 5.00E−06 |
| 7645_Chr15_33901557_33901681 | Seq_GeCjCas9.OGB.20plus_chr1_237600602_237600625 | 95.65 | 23 | 5.00E−06 |
| 7645_Chr15_33901557_33901681 | Seq_GeCjCas9.OGB.20plus_chr9_66469976_66469999 | 100 | 22 | 9.00E−08 |
| 7802_Chr15_93350891_93351001 | Seq_GeCjCas9.OGB.20plus_chr5_57183007_57183030 | 95.65 | 23 | 5.00E−06 |
| 7802_Chr15_93350891_93351001 | Seq_GeCjCas9.OGB.20plus_chr1_237600602_237600625 | 95.65 | 23 | 5.00E−06 |
| 7802_Chr15_93350891_93351001 | Seq_GeCjCas9.OGB.20plus_chr9_66469976_66469999 | 100 | 22 | 8.00E−08 |
| 7903_Chr16_14391749_14391872 | Seq_GeCjCas9.OGB.20plus_chr5_57183007_57183030 | 95.65 | 23 | 5.00E−06 |
| 7903_Chr16_14391749_14391872 | Seq_GeCjCas9.OGB.20plus_chr1_237600602_237600625 | 95.65 | 23 | 5.00E−06 |
| 7903_Chr16_14391749_14391872 | Seq_GeCjCas9.OGB.20plus_chr9_66469976_66469999 | 100 | 22 | 9.00E−08 |
| 7936_Chr16_21584700_21584808 | Seq_GeCjCas9.OGB.20plus_chr9_66469976_66469999 | 100 | 23 | 2.00E−08 |
| 7936_Chr16_21584700_21584808 | Seq_GeCjCas9.OGB.20plus_chr9_66469976_66469999 | 95.65 | 23 | 5.00E−06 |
| 7936_Chr16_21584700_21584808 | Seq_GeCjCas9.OGB.20plus_chr2_54032605_54032628 | 95.65 | 23 | 5.00E−06 |
| 7936_Chr16_21584700_21584808 | Seq_GeCjCas9.OGB.20plus_chr1_150539667_150539690 | 95.65 | 23 | 5.00E−06 |
| 7948_Chr16_27851607_27851699 | Seq_GeCjCas9.OGB.20plus_chr9_66469976_66469999 | 95.65 | 23 | 4.00E−06 |
| 7948_Chr16_27851607_27851699 | Seq_GeCjCas9.OGB.20plus_chr1_150539667_150539690 | 95.65 | 23 | 4.00E−06 |
| 796_Chr1_245138484_245138592 | Seq_GeCjCas9.OGB.20plus_chr9_66469976_66469999 | 95.65 | 23 | 5.00E−06 |
| 7988_Chr16_57839780_57839868 | Seq_GeCjCas9.OGB.20plus_chr3_134188505_134188528 | 95.65 | 23 | 4.00E−06 |
| 8012_Chr16_67844992_67845092 | Seq_GeCjCas9.OGB.20plus_chr5_57183007_57183030 | 95.65 | 23 | 4.00E−06 |
| 8012_Chr16_67844992_67845092 | Seq_GeCjCas9.OGB.20plus_chr1_237600602_237600625 | 95.65 | 23 | 4.00E−06 |
| 8012_Chr16_67844992_67845092 | Seq_GeCjCas9.OGB.20plus_chr9_66469976_66469999 | 100 | 22 | 7.00E−08 |
| 8022_Chr16_72062630_72062754 | Seq_GeCjCas9.OGB.20plus_chr9_66469976_66469999 | 100 | 23 | 2.00E−08 |
| 8022_Chr16_72062630_72062754 | Seq_GeCjCas9.OGB.20plus_chr9_66469976_66469999 | 95.65 | 23 | 5.00E−06 |
| 8022_Chr16_72062630_72062754 | Seq_GeCjCas9.OGB.20plus_chr2_54032605_54032628 | 95.65 | 23 | 5.00E−06 |
| 8022_Chr16_72062630_72062754 | Seq_GeCjCas9.OGB.20plus_chr1_150539667_150539690 | 95.65 | 23 | 5.00E−06 |
| 8218_Chr17_29052153_29052275 | Seq_GeCjCas9.OGB.20plus_chr5_57183007_57183030 | 95.65 | 23 | 5.00E−06 |
| 8218_Chr17_29052153_29052275 | Seq_GeCjCas9.OGB.20plus_chr1_237600602_237600625 | 95.65 | 23 | 5.00E−06 |
| 8218_Chr17_29052153_29052275 | Seq_GeCjCas9.OGB.20plus_chr9_66469976_66469999 | 100 | 22 | 8.00E−08 |
| 8258_Chr17_44126814_44126916 | Seq_GeCjCas9.OGB.20plus_chr9_66469976_66469999 | 100 | 23 | 2.00E−08 |
| 8258_Chr17_44126814_44126916 | Seq_GeCjCas9.OGB.20plus_chr2_54032605_54032628 | 95.65 | 23 | 4.00E−06 |
| 8258_Chr17_44126814_44126916 | Seq_GeCjCas9.OGB.20plus_chr1_150539667_150539690 | 95.65 | 23 | 4.00E−06 |
| 8269_Chr17_46994135_46994227 | Seq_GeCjCas9.OGB.20plus_chr11_77993558_77993581 | 95.65 | 23 | 4.00E−06 |
| 8309_Chr17_57323810_57323931 | Seq_GeCjCas9.OGB.20plus_chr5_31469271_31469294 | 95.65 | 23 | 5.00E−06 |
| 8309_Chr17_57323810_57323931 | Seq_GeCjCas9.OGB.20plus_chr12_47579379_47579402 | 95.65 | 23 | 5.00E−06 |
| 8311_Chr17_58019456_58019571 | Seq_GeCjCas9.OGB.20plus_chr9_66469976_66469999 | 100 | 23 | 2.00E−08 |
| 8311_Chr17_58019456_58019571 | Seq_GeCjCas9.OGB.20plus_chr2_54032605_54032628 | 95.65 | 23 | 5.00E−06 |
| 8311_Chr17_58019456_58019571 | Seq_GeCjCas9.OGB.20plus_chr1_150539667_150539690 | 95.65 | 23 | 5.00E−06 |
| 8376_Chr17_76278190_76278306 | Seq_GeCjCas9.OGB.20plus_chr9_66469976_66469999 | 100 | 23 | 2.00E−08 |
| 8376_Chr17_76278190_76278306 | Seq_GeCjCas9.OGB.20plus_chr9_66469976_66469999 | 95.65 | 23 | 5.00E−06 |
| 8376_Chr17_76278190_76278306 | Seq_GeCjCas9.OGB.20plus_chr2_54032605_54032628 | 95.65 | 23 | 5.00E−06 |
| 8376_Chr17_76278190_76278306 | Seq_GeCjCas9.OGB.20plus_chr1_150539667_150539690 | 95.65 | 23 | 5.00E−06 |
| 8378_Chr17_76790747_76790854 | Seq_GeCjCas9.OGB.20plus_chr9_66469976_66469999 | 100 | 23 | 2.00E−08 |
| 8378_Chr17_76790747_76790854 | Seq_GeCjCas9.OGB.20plus_chr2_54032605_54032628 | 95.65 | 23 | 5.00E−06 |
| 8378_Chr17_76790747_76790854 | Seq_GeCjCas9.OGB.20plus_chr1_150539667_150539690 | 95.65 | 23 | 5.00E−06 |
| 8594_Chr18_67847548_67847684 | Seq_GeCjCas9.OGB.20plus_chr11_77993558_77993581 | 95.65 | 23 | 6.00E−06 |
| 8680_Chr19_1887080_1887158 | Seq_GeCjCas9.OGB.20plus_chr5_31469271_31469294 | 95.65 | 23 | 3.00E−06 |
| 8680_Chr19_1887080_1887158 | Seq_GeCjCas9.OGB.20plus_chr12_47579379_47579402 | 95.65 | 23 | 3.00E−06 |
| 8703_Chr19_6442810_6442927 | Seq_GeCjCas9.OGB.20plus_chr5_57183007_57183030 | 95.65 | 23 | 5.00E−06 |
| 8703_Chr19_6442810_6442927 | Seq_GeCjCas9.OGB.20plus_chr5_57183007_57183030 | 95.65 | 23 | 5.00E−06 |
| 8703_Chr19_6442810_6442927 | Seq_GeCjCas9.OGB.20plus_chr1_237600602_237600625 | 95.65 | 23 | 5.00E−06 |
| 8703_Chr19_6442810_6442927 | Seq_GeCjCas9.OGB.20plus_chr1_237600602_237600625 | 95.65 | 23 | 5.00E−06 |
| 8703_Chr19_6442810_6442927 | Seq_GeCjCas9.OGB.20plus_chr9_66469976_66469999 | 100 | 22 | 8.00E−08 |
| 8703_Chr19_6442810_6442927 | Seq_GeCjCas9.OGB.20plus_chr9_66469976_66469999 | 100 | 22 | 8.00E−08 |
| 8703_Chr19_6442810_6442927 | Seq_GeCjCas9.OGB.20plus_chr17_4861121_4861144 | 95.65 | 23 | 5.00E−06 |
| 8773_Chr19_24585850_24585981 | Seq_GeCjCas9.OGB.20plus_chr7_61968699_61968722 | 95.65 | 23 | 6.00E−06 |
| 8773_Chr19_24585850_24585981 | Seq_GeCjCas9.OGB.20plus_chr21_10772927_10772950 | 95.65 | 23 | 6.00E−06 |
| 8773_Chr19_24585850_24585981 | Seq_GeCjCas9.OGB.20plus_chr18_18519302_18519325 | 95.65 | 23 | 6.00E−06 |
| 8991_Chr20_31299717_31299831 | Seq_GeCjCas9.OGB.20plus_chr1_237600602_237600625 | 95.65 | 23 | 5.00E−06 |
| 9041_Chr20_47791131_47791244 | Seq_GeCjCas9.OGB.20plus_chr5_57183007_57183030 | 95.65 | 23 | 5.00E−06 |
| 9041_Chr20_47791131_47791244 | Seq_GeCjCas9.OGB.20plus_chr1_237600602_237600625 | 95.65 | 23 | 5.00E−06 |
| 9041_Chr20_47791131_47791244 | Seq_GeCjCas9.OGB.20plus_chr9_66469976_66469999 | 100 | 22 | 8.00E−08 |
| 9338_Chr22_25198710_25198837 | Seq_GeCjCas9.OGB.20plus_chr5_57183007_57183030 | 95.65 | 23 | 5.00E−06 |
| 9338_Chr22_25198710_25198837 | Seq_GeCjCas9.OGB.20plus_chr1_237600602_237600625 | 95.65 | 23 | 5.00E−06 |
| 9338_Chr22_25198710_25198837 | Seq_GeCjCas9.OGB.20plus_chr9_66469976_66469999 | 100 | 22 | 9.00E−08 |
| 9345_Chr22_29847209_29847300 | Seq_GeCjCas9.OGB.20plus_chr5_57183007_57183030 | 95.65 | 23 | 4.00E−06 |
| 9345_Chr22_29847209_29847300 | Seq_GeCjCas9.OGB.20plus_chr1_237600602_237600625 | 95.65 | 23 | 4.00E−06 |
| 9345_Chr22_29847209_29847300 | Seq_GeCjCas9.OGB.20plus_chr9_66469976_66469999 | 100 | 22 | 6.00E−08 |
| 9352_Chr22_31657195_31657308 | Seq_GeCjCas9.OGB.20plus_chr5_57183007_57183030 | 95.65 | 23 | 5.00E−06 |
| 9352_Chr22_31657195_31657308 | Seq_GeCjCas9.OGB.20plus_chr1_237600602_237600625 | 95.65 | 23 | 5.00E−06 |
| 9352_Chr22_31657195_31657308 | Seq_GeCjCas9.OGB.20plus_chr9_66469976_66469999 | 100 | 22 | 8.00E−08 |
| 9378_Chr22_39847505_39847612 | Seq_GeCjCas9.OGB.20plus_chr5_57183007_57183030 | 95.65 | 23 | 5.00E−06 |
| 9378_Chr22_39847505_39847612 | Seq_GeCjCas9.OGB.20plus_chr1_237600602_237600625 | 95.65 | 23 | 5.00E−06 |
| 9378_Chr22_39847505_39847612 | Seq_GeCjCas9.OGB.20plus_chr9_66469976_66469999 | 100 | 22 | 7.00E−08 |
| 9385_Chr22_41341585_41341694 | Seq_GeCjCas9.OGB.20plus_chr9_66469976_66469999 | 95.65 | 23 | 5.00E−06 |
| 9390_Chr22_41797025_41797156 | Seq_GeCjCas9.OGB.20plus_chr9_66469976_66469999 | 100 | 23 | 2.00E−08 |
| 9390_Chr22_41797025_41797156 | Seq_GeCjCas9.OGB.20plus_chr2_54032605_54032628 | 95.65 | 23 | 6.00E−06 |
| 9390_Chr22_41797025_41797156 | Seq_GeCjCas9.OGB.20plus_chr1_150539667_150539690 | 95.65 | 23 | 6.00E−06 |
| 9393_Chr22_42647977_42648072 | Seq_GeCjCas9.OGB.20plus_chr11_77993558_77993581 | 95.65 | 23 | 4.00E−06 |
| 9397_Chr22_43417966_43418096 | Seq_GeCjCas9.OGB.20plus_chr9_66469976_66469999 | 100 | 22 | 9.00E−08 |
| 9397_Chr22_43417966_43418096 | Seq_GeCjCas9.OGB.20plus_chr9_66469976_66469999 | 100 | 22 | 9.00E−08 |
| 9462_ChrX_1371686_1371788 | Seq_GeCjCas9.OGB.20plus_chr5_57183007_57183030 | 95.65 | 23 | 4.00E−06 |
| 9462_ChrX_1371686_1371788 | Seq_GeCjCas9.OGB.20plus_chr1_237600602_237600625 | 95.65 | 23 | 4.00E−06 |
| 9462_ChrX_1371686_1371788 | Seq_GeCjCas9.OGB.20plus_chr9_66469976_66469999 | 100 | 22 | 7.00E−08 |
| 9468_ChrX_2006143_2006265 | Seq_GeCjCas9.OGB.20plus_chr15_57336567_57336590 | 95.65 | 23 | 5.00E−06 |
| 9515_ChrX_20205143_20205251 | Seq_GeCjCas9.OGB.20plus_chr15_57336567_57336590 | 95.65 | 23 | 5.00E−06 |
| 9556_ChrX_38313149_38313274 | Seq_GeCjCas9.OGB.20plus_chr9_66469976_66469999 | 100 | 23 | 2.00E−08 |
| 9556_ChrX_38313149_38313274 | Seq_GeCjCas9.OGB.20plus_chr2_54032605_54032628 | 95.65 | 23 | 5.00E−06 |
| 9556_ChrX_38313149_38313274 | Seq_GeCjCas9.OGB.20plus_chr1_150539667_150539690 | 95.65 | 23 | 5.00E−06 |
| 9563_ChrX_40853707_40853812 | Seq_GeCjCas9.OGB.20plus_chr9_66469976_66469999 | 100 | 23 | 2.00E−08 |
| 9563_ChrX_40853707_40853812 | Seq_GeCjCas9.OGB.20plus_chr5_57183007_57183030 | 95.65 | 23 | 4.00E−06 |
| 9563_ChrX_40853707_40853812 | Seq_GeCjCas9.OGB.20plus_chr2_54032605_54032628 | 95.65 | 23 | 4.00E−06 |
| 9563_ChrX_40853707_40853812 | Seq_GeCjCas9.OGB.20plus_chr1_237600602_237600625 | 95.65 | 23 | 4.00E−06 |
| 9563_ChrX_40853707_40853812 | Seq_GeCjCas9.OGB.20plus_chr1_150539667_150539690 | 95.65 | 23 | 4.00E−06 |
| 9563_ChrX_40853707_40853812 | Seq_GeCjCas9.OGB.20plus_chr9_66469976_66469999 | 100 | 22 | 7.00E−08 |
| 957_Chr2_34380079_34380204 | Seq_GeCjCas9.OGB.20plus_chr3_134188505_134188528 | 100 | 23 | 2.00E−08 |
| 957_Chr2_34380079_34380204 | Seq_GeCjCas9.OGB.20plus_chr3_134188505_134188528 | 95.65 | 23 | 5.00E−06 |
| 9611_ChrX_58490619_58490729 | Seq_GeCjCas9.OGB.20plus_chr18_18516861_18516884 | 95.65 | 23 | 5.00E−06 |
| 980_Chr2_39185999_39186100 | Seq_GeCjCas9.OGB.20plus_chr3_134188505_134188528 | 95.65 | 23 | 4.00E−06 |
| 9881_ChrY_1321686_1321788 | Seq_GeCjCas9.OGB.20plus_chr5_57183007_57183030 | 95.65 | 23 | 4.00E−06 |
| 9881_ChrY_1321686_1321788 | Seq_GeCjCas9.OGB.20plus_chr1_237600602_237600625 | 95.65 | 23 | 4.00E−06 |
| 9881_ChrY_1321686_1321788 | Seq_GeCjCas9.OGB.20plus_chr9_66469976_66469999 | 100 | 22 | 7.00E−08 |
| 9887_ChrY_1956143_1956265 | Seq_GeCjCas9.OGB.20plus_chr15_57336567_57336590 | 95.65 | 23 | 5.00E−06 |
| Cj Cas9 inf transf minus strand | | | | |
| 5839_Chr10_70283722_70283823 | Seq_GeCjCas9.OGB.20minus_chr3_160851802_160851825 | 100 | 22 | 7.00E−08 |
| 7590_Chr15_20014699_20014838 | Seq_GeCjCas9.OGB.20minus_chr20_26303264_26303287 | 100 | 22 | 1.00E−07 |
| 7590_Chr15_20014699_20014838 | Seq_GeCjCas9.OGB.20minus_chr20_26303264_26303287 | 100 | 22 | 1.00E−07 |
| 1109_Chr2_85737752_85738008 | Seq_GeCjCas9.OGB.20minus_chr17_4861104_4861127 | 100 | 23 | 4.00E−08 |
| 1109_Chr2_85737752_85738008 | Seq_GeCjCas9.OGB.20minus_chr17_4861104_4861127 | 100 | 23 | 4.00E−08 |
| 130_Chr1_28173679_28173782 | Seq_GeCjCas9.OGB.20minus_chr12_14461527_14461550 | 100 | 23 | 2.00E−08 |
| 1419_Chr2_202729300_202729405 | Seq_GeCjCas9.OGB.20minus_chr4_25224538_25224561 | 95.65 | 23 | 4.00E−06 |
| 1565_Chr2_242262774_242262909 | Seq_GeCjCas9.OGB.20minus_chr5_31469254_31469277 | 95.65 | 23 | 6.00E−06 |
| 1565_Chr2_242262774_242262909 | Seq_GeCjCas9.OGB.20minus_chr5_31469254_31469277 | 95.65 | 23 | 6.00E−06 |
| 1565_Chr2_242262774_242262909 | Seq_GeCjCas9.OGB.20minus_chr12_633017_633040 | 95.65 | 23 | 6.00E−06 |
| 1565_Chr2_242262774_242262909 | Seq_GeCjCas9.OGB.20minus_chr12_633017_633040 | 95.65 | 23 | 6.00E−06 |
| 1611_Chr3_14667912_14668025 | Seq_GeCjCas9.OGB.20minus_chr17_4861104_4861127 | 100 | 23 | 2.00E−08 |
| 1672_Chr3_32692995_32693124 | Seq_GeCjCas9.OGB.20minus_chr14_38384381_38384404 | 95.65 | 23 | 6.00E−06 |
| 1717_Chr3_47430996_47431114 | Seq_GeCjCas9.OGB.20minus_chr14_105785516_105785539 | 95.65 | 23 | 5.00E−06 |
| 1889_Chr3_109079607_109079696 | Seq_GeCjCas9.OGB.20minus_chr5_31469254_31469277 | 95.65 | 23 | 4.00E−06 |
| 1889_Chr3_109079607_109079696 | Seq_GeCjCas9.OGB.20minus_chr12_633017_633040 | 95.65 | 23 | 4.00E−06 |
| 2131_Chr3_185312627_185312778 | Seq_GeCjCas9.OGB.20minus_chr5_31469254_31469277 | 95.65 | 23 | 7.00E−06 |
| 2131_Chr3_185312627_185312778 | Seq_GeCjCas9.OGB.20minus_chr12_633017_633040 | 95.65 | 23 | 7.00E−06 |
| 2231_Chr4_3335028_3335150 | Seq_GeCjCas9.OGB.20minus_chr4_25224538_25224561 | 95.65 | 23 | 5.00E−06 |
| 2333_Chr4_25484480_25484588 | Seq_GeCjCas9.OGB.20minus_chr12_6328449_6328472 | 95.65 | 23 | 5.00E−06 |
| 2750_Chr4_163803118_163803209 | Seq_GeCjCas9.OGB.20minus_chr10_33287520_33287543 | 100 | 23 | 2.00E−08 |
| 2765_Chr4_165781005_165781116 | Seq_GeCjCas9.OGB.20minus_chr1_150539650_150539673 | 95.65 | 23 | 5.00E−06 |
| 2863_Chr4_186334311_186334430 | Seq_GeCjCas9.OGB.20minus_chr11_77993541_77993564 | 95.65 | 23 | 5.00E−06 |
| 3081_Chr5_37367004_37367113 | Seq_GeCjCas9.OGB.20minus_chr4_25224538_25224561 | 95.65 | 23 | 5.00E−06 |
| 3677_Chr6_52204775_52204885 | Seq_GeCjCas9.OGB.20minus_chr17_4861104_4861127 | 95.65 | 23 | 5.00E−06 |
| 3696_Chr6_58244470_58244582 | Seq_GeCjCas9.OGB.20minus_chr17_4861104_4861127 | 100 | 23 | 2.00E−08 |
| 3696_Chr6_58244470_58244582 | Seq_GeCjCas9.OGB.20minus_chr17_4861104_4861127 | 100 | 23 | 2.00E−08 |
| 3716_Chr6_64381823_64381962 | Seq_GeCjCas9.OGB.20minus_chr22_20325962_20325985 | 100 | 23 | 2.00E−08 |
| 4012_Chr6_158992376_158992468 | Seq_GeCjCas9.OGB.20minus_chr4_25224538_25224561 | 95.65 | 23 | 4.00E−06 |
| 4170_Chr7_21558982_21559129 | Seq_GeCjCas9.OGB.20minus_chr5_31469254_31469277 | 95.65 | 23 | 6.00E−06 |
| 4170_Chr7_21558982_21559129 | Seq_GeCjCas9.OGB.20minus_chr5_31469254_31469277 | 95.65 | 23 | 6.00E−06 |
| 4170_Chr7_21558982_21559129 | Seq_GeCjCas9.OGB.20minus_chr12_633017_633040 | 95.65 | 23 | 6.00E−06 |
| 4170_Chr7_21558982_21559129 | Seq_GeCjCas9.OGB.20minus_chr12_633017_633040 | 95.65 | 23 | 6.00E−06 |
| 4256_Chr7_62033801_62033942 | Seq_GeCjCas9.OGB.20minus_chr9_67320800_67320823 | 95.65 | 23 | 6.00E−06 |
| 4256_Chr7_62033801_62033942 | Seq_GeCjCas9.OGB.20minus_chr2_92316183_92316206 | 95.65 | 23 | 6.00E−06 |
| 4256_Chr7_62033801_62033942 | Seq_GeCjCas9.OGB.20minus_chr2_92316183_92316206 | 95.65 | 23 | 6.00E−06 |
| 4258_Chr7_62986233_62986335 | Seq_GeCjCas9.OGB.20minus_chr17_4861104_4861127 | 100 | 23 | 2.00E−08 |
| 4259_Chr7_63025117_63025233 | Seq_GeCjCas9.OGB.20minus_chr4_25224538_25224561 | 95.65 | 23 | 5.00E−06 |
| 4268_Chr7_65634491_65634593 | Seq_GeCjCas9.OGB.20minus_chr17_4861104_4861127 | 95.65 | 23 | 4.00E−06 |
| 4323_Chr7_75182490_75182581 | Seq_GeCjCas9.OGB.20minus_chr17_4861104_4861127 | 100 | 23 | 2.00E−08 |
| 4331_Chr7_76985344_76985448 | Seq_GeCjCas9.OGB.20minus_chr17_4861104_4861127 | 100 | 23 | 2.00E−08 |
| 5043_Chr8_103816263_103816365 | Seq_GeCjCas9.OGB.20minus_chr14_105785516_105785539 | 95.65 | 23 | 4.00E−06 |
| 5421_Chr9_98070799_98070929 | Seq_GeCjCas9.OGB.20minus_chr4_25224538_25224561 | 95.65 | 23 | 6.00E−06 |
| 5526_Chr9_134170222_134170294 | Seq_GeCjCas9.OGB.20minus_chr17_4861104_4861127 | 95.65 | 23 | 3.00E−06 |
| 5636_Chr10_5923903_5924012 | Seq_GeCjCas9.OGB.20minus_chr4_25224538_25224561 | 95.65 | 23 | 5.00E−06 |
| 5732_Chr10_35154125_35154258 | Seq_GeCjCas9.OGB.20minus_chr5_31469254_31469277 | 100 | 23 | 2.00E−08 |
| 5732_Chr10_35154125_35154258 | Seq_GeCjCas9.OGB.20minus_chr12_633017_633040 | 100 | 23 | 2.00E−08 |
| 5908_Chr10_92437211_92437345 | Seq_GeCjCas9.OGB.20minus_chr5_31469254_31469277 | 100 | 23 | 2.00E−08 |
| 5908_Chr10_92437211_92437345 | Seq_GeCjCas9.OGB.20minus_chr12_633017_633040 | 100 | 23 | 2.00E−08 |
| 6366_Chr11_97961502_97961616 | Seq_GeCjCas9.OGB.20minus_chr17_4861104_4861127 | 100 | 23 | 2.00E−08 |
| 6622_Chr12_34685662_34685768 | Seq_GeCjCas9.OGB.20minus_chr1_121485296_121485319 | 95.65 | 23 | 4.00E−06 |
| 67_Chr1_8320950_8321039 | Seq_GeCjCas9.OGB.20minus_chr4_25224538_25224561 | 95.65 | 23 | 4.00E−06 |
| 6740_Chr12_77287454_77287545 | Seq_GeCjCas9.OGB.20minus_chr17_4861104_4861127 | 95.65 | 23 | 4.00E−06 |
| 6871_Chr12_122174321_122174450 | Seq_GeCjCas9.OGB.20minus_chr4_25224538_25224561 | 95.65 | 23 | 6.00E−06 |
| 6871_Chr12_122174321_122174450 | Seq_GeCjCas9.OGB.20minus_chr4_25224538_25224561 | 95.65 | 23 | 6.00E−06 |
| 6877_Chr12_123951337_123951461 | Seq_GeCjCas9.OGB.20minus_chr4_25224538_25224561 | 95.65 | 23 | 5.00E−06 |
| 6882_Chr12_125436221_125436328 | Seq_GeCjCas9.OGB.20minus_chr11_84152405_84152428 | 100 | 23 | 2.00E−08 |
| 6882_Chr12_125436221_125436328 | Seq_GeCjCas9.OGB.20minus_chr11_84152405_84152428 | 95.65 | 23 | 5.00E−06 |
| 6882_Chr12_125436221_125436328 | Seq_GeCjCas9.OGB.20minus_chr1_102866197_102866220 | 100 | 23 | 2.00E−08 |
| 6882_Chr12_125436221_125436328 | Seq_GeCjCas9.OGB.20minus_chr1_102866197_102866220 | 95.65 | 23 | 5.00E−06 |
| 7590_Chr15_20014699_20014838 | Seq_GeCjCas9.OGB.20minus_chr10_42400353_42400376 | 95.65 | 23 | 6.00E−06 |
| 7802_Chr15_93350891_93351001 | Seq_GeCjCas9.OGB.20minus_chr17_4861104_4861127 | 100 | 23 | 2.00E−08 |
| 8012_Chr16_67844992_67845092 | Seq_GeCjCas9.OGB.20minus_chr17_4861104_4861127 | 100 | 23 | 2.00E−08 |
| 8218_Chr17_29052153_29052275 | Seq_GeCjCas9.OGB.20minus_chr17_4861104_4861127 | 100 | 23 | 2.00E−08 |
| 8248_Chr17_40906107_40906267 | Seq_GeCjCas9.OGB.20minus_chr5_31469254_31469277 | 100 | 23 | 3.00E−08 |
| 8248_Chr17_40906107_40906267 | Seq_GeCjCas9.OGB.20minus_chr5_31469254_31469277 | 95.65 | 23 | 7.00E−06 |
| 8248_Chr17_40906107_40906267 | Seq_GeCjCas9.OGB.20minus_chr12_633017_633040 | 100 | 23 | 3.00E−08 |
| 8248_Chr17_40906107_40906267 | Seq_GeCjCas9.OGB.20minus_chr12_633017_633040 | 95.65 | 23 | 7.00E−06 |
| 8258_Chr17_44126814_44126916 | Seq_GeCjCas9.OGB.20minus_chr4_25224538_25224561 | 95.65 | 23 | 4.00E−06 |
| 8311_Chr17_58019456_58019571 | Seq_GeCjCas9.OGB.20minus_chr4_25224538_25224561 | 95.65 | 23 | 5.00E−06 |
| 8515_Chr18_36425716_36425796 | Seq_GeCjCas9.OGB.20minus_chr10_70837929_70837952 | 95.65 | 23 | 3.00E−06 |
| 8703_Chr19_6442810_6442927 | Seq_GeCjCas9.OGB.20minus_chr17_4861104_4861127 | 100 | 23 | 2.00E−08 |
| 8703_Chr19_6442810_6442927 | Seq_GeCjCas9.OGB.20minus_chr17_4861104_4861127 | 100 | 23 | 2.00E−08 |
| 8773_Chr19_24585850_24585981 | Seq_GeCjCas9.OGB.20minus_chr21_10772910_10772933 | 100 | 23 | 2.00E−08 |
| 8799_Chr19_36687618_36687728 | Seq_GeCjCas9.OGB.20minus_chr11_84152405_84152428 | 100 | 23 | 2.00E−08 |
| 8799_Chr19_36687618_36687728 | Seq_GeCjCas9.OGB.20minus_chr1_102866197_102866220 | 95.65 | 23 | 5.00E−06 |
| 921_Chr2_22196550_22196650 | Seq_GeCjCas9.OGB.20minus_chr5_31469254_31469277 | 100 | 23 | 2.00E−08 |
| 921_Chr2_22196550_22196650 | Seq_GeCjCas9.OGB.20minus_chr12_633017_633040 | 100 | 23 | 2.00E−08 |
| 9338_Chr22_25198710_25198837 | Seq_GeCjCas9.OGB.20minus_chr5_31469254_31469277 | 100 | 23 | 2.00E−08 |
| 9338_Chr22_25198710_25198837 | Seq_GeCjCas9.OGB.20minus_chr5_31469254_31469277 | 95.65 | 23 | 5.00E−06 |
| 9338_Chr22_25198710_25198837 | Seq_GeCjCas9.OGB.20minus_chr17_4861104_4861127 | 100 | 23 | 2.00E−08 |
| 9338_Chr22_25198710_25198837 | Seq_GeCjCas9.OGB.20minus_chr12_633017_633040 | 100 | 23 | 2.00E−08 |
| 9338_Chr22_25198710_25198837 | Seq_GeCjCas9.OGB.20minus_chr12_633017_633040 | 95.65 | 23 | 5.00E−06 |
| 9389_Chr22_41784175_41784446 | Seq_GeCjCas9.OGB.20minus_chr5_31469254_31469277 | 100 | 23 | 5.00E−08 |
| 9389_Chr22_41784175_41784446 | Seq_GeCjCas9.OGB.20minus_chr12_633017_633040 | 100 | 23 | 5.00E−08 |
| 9390_Chr22_41797025_41797156 | Seq_GeCjCas9.OGB.20minus_chr5_31469254_31469277 | 95.65 | 23 | 6.00E−06 |
| 9390_Chr22_41797025_41797156 | Seq_GeCjCas9.OGB.20minus_chr4_25224538_25224561 | 95.65 | 23 | 6.00E−06 |
| 9390_Chr22_41797025_41797156 | Seq_GeCjCas9.OGB.20minus_chr12_633017_633040 | 95.65 | 23 | 6.00E−06 |
| 9403_Chr22_45070254_45070367 | Seq_GeCjCas9.OGB.20minus_chr2_61396269_61396292 | 95.65 | 23 | 5.00E−06 |
| 9462_ChrX_1371686_1371788 | Seq_GeCjCas9.OGB.20minus_chr17_4861104_4861127 | 100 | 23 | 2.00E−08 |
| 9468_ChrX_2006143_2006265 | Seq_GeCjCas9.OGB.20minus_chr5_31469254_31469277 | 100 | 23 | 2.00E−08 |
| 9468_ChrX_2006143_2006265 | Seq_GeCjCas9.OGB.20minus_chr12_633017_633040 | 100 | 23 | 2.00E−08 |
| 9468_ChrX_2006143_2006265 | Seq_GeCjCas9.OGB.20minus_chr12_47579362_47579385 | 100 | 23 | 2.00E−08 |
| 9468_ChrX_2006143_2006265 | Seq_GeCjCas9.OGB.20minus_chr3_134188488_134188511 | 95.65 | 23 | 5.00E−06 |
| 9556_ChrX_38313149_38313274 | Seq_GeCjCas9.OGB.20minus_chr4_25224538_25224561 | 95.65 | 23 | 5.00E−06 |
| 9563_ChrX_40853707_40853812 | Seq_GeCjCas9.OGB.20minus_chr17_4861104_4861127 | 100 | 23 | 2.00E−08 |
| 9563_ChrX_40853707_40853812 | Seq_GeCjCas9.OGB.20minus_chr5_31469254_31469277 | 95.65 | 23 | 4.00E−06 |
| 9563_ChrX_40853707_40853812 | Seq_GeCjCas9.OGB.20minus_chr4_25224538_25224561 | 95.65 | 23 | 4.00E−06 |
| 9563_ChrX_40853707_40853812 | Seq_GeCjCas9.OGB.20minus_chr12_633017_633040 | 95.65 | 23 | 4.00E−06 |
| 9611_ChrX_58490619_58490729 | Seq_GeCjCas9.OGB.20minus_chr7_61969174_61969197 | 95.65 | 23 | 5.00E−06 |
| 9611_ChrX_58490619_58490729 | Seq_GeCjCas9.OGB.20minus_chr7_61969166_61969189 | 95.65 | 23 | 5.00E−06 |
| 9611_ChrX_58490619_58490729 | Seq_GeCjCas9.OGB.20minus_chr2_92305578_92305601 | 95.65 | 23 | 5.00E−06 |
| 9789_ChrX_126005872_126006039 | Seq_GeCjCas9.OGB.20minus_chr5_15414511_15414534 | 100 | 23 | 3.00E−08 |
| 9831_ChrX_141577038_141577166 | Seq_GeCjCas9.OGB.20minus_chr20_54630895_54630918 | 95.65 | 23 | 6.00E−06 |
| 9881_ChrY_1321686_1321788 | Seq_GeCjCas9.OGB.20minus_chr17_4861104_4861127 | 100 | 23 | 2.00E−08 |
| 9887_ChrY_1956143_1956265 | Seq_GeCjCas9.OGB.20minus_chr5_31469254_31469277 | 100 | 23 | 2.00E−08 |
| 9887_ChrY_1956143_1956265 | Seq_GeCjCas9.OGB.20minus_chr12_633017_633040 | 100 | 23 | 2.00E−08 |
| 9887_ChrY_1956143_1956265 | Seq_GeCjCas9.OGB.20minus_chr12_47579362_47579385 | 100 | 23 | 2.00E−08 |
| 9887_ChrY_1956143_1956265 | Seq_GeCjCas9.OGB.20minus_chr3_134188488_134188511 | 95.65 | 23 | 5.00E−06 |
| 130_Chr1_28173679_28173782 | Seq_GeCjCas9.OGB.20minus_chr6_57173198_57173221 | 100 | 22 | 7.00E−08 |
| 1672_Chr3_32692995_32693124 | Seq_GeCjCas9.OGB.20minus_chr8_141501469_141501492 | 100 | 22 | 9.00E−08 |
| 3081_Chr5_37367004_37367113 | Seq_GeCjCas9.OGB.20minus_chr17_4861104_4861127 | 100 | 22 | 7.00E−08 |
| 4012_Chr6_158992376_158992468 | Seq_GeCjCas9.OGB.20minus_chr17_4861104_4861127 | 100 | 22 | 6.00E−08 |
| 5421_Chr9_98070799_98070929 | Seq_GeCjCas9.OGB.20minus_chr17_4861104_4861127 | 100 | 22 | 9.00E−08 |
| 5636_Chr10_5923903_5924012 | Seq_GeCjCas9.OGB.20minus_chr17_4861104_4861127 | 100 | 22 | 7.00E−08 |
| 6008_Chr10_126470795_126470906 | Seq_GeCjCas9.OGB.20minus_chr14_38384381_38384404 | 100 | 22 | 8.00E−08 |
| 67_Chr1_8320950_8321039 | Seq_GeCjCas9.OGB.20minus_chr17_4861104_4861127 | 100 | 22 | 6.00E−08 |
| 6871_Chr12_122174321_122174450 | Seq_GeCjCas9.OGB.20minus_chr17_4861104_4861127 | 100 | 22 | 9.00E−08 |
| 6871_Chr12_122174321_122174450 | Seq_GeCjCas9.OGB.20minus_chr17_4861104_4861127 | 100 | 22 | 9.00E−08 |
| 6877_Chr12_123951337_123951461 | Seq_GeCjCas9.OGB.20minus_chr17_4861104_4861127 | 100 | 22 | 9.00E−08 |
| 7342_Chr14_28266246_28266360 | Seq_GeCjCas9.OGB.20minus_chr8_141501469_141501492 | 100 | 22 | 8.00E−08 |
| 8258_Chr17_44126814_44126916 | Seq_GeCjCas9.OGB.20minus_chr17_4861104_4861127 | 100 | 22 | 7.00E−08 |
| 8311_Chr17_58019456_58019571 | Seq_GeCjCas9.OGB.20minus_chr17_4861104_4861127 | 100 | 22 | 8.00E−08 |
| 9390_Chr22_41797025_41797156 | Seq_GeCjCas9.OGB.20minus_chr17_4861104_4861127 | 100 | 22 | 9.00E−08 |
| 9556_ChrX_38313149_38313274 | Seq_GeCjCas9.OGB.20minus_chr17_4861104_4861127 | 100 | 22 | 9.00E−08 |
| 9563_ChrX_40853707_40853812 | Seq_GeCjCas9.OGB.20minus_chr17_4861104_4861127 | 100 | 22 | 7.00E−08 |
| 5636_Chr10_5923903_5924012 | Seq_GeCjCas9.OGB.20minus_chr14_105785516_105785539 | 100 | 22 | 7.00E−08 |
| 6222_Chr11_48829963_48830063 | Seq_GeCjCas9.OGB.20minus_chr16_35222615_35222638 | 100 | 22 | 7.00E−08 |
| 9460_ChrX_974288_974554 | Seq_GeCjCas9.OGB.20minus_chr17_4861104_4861127 | 100 | 22 | 2.00E−07 |
| 9879_ChrY_924288_924554 | Seq_GeCjCas9.OGB.20minus_chr17_4861104_4861127 | 100 | 22 | 2.00E−07 |
| 1042_Chr2_61856095_61856191 | Seq_GeCjCas9.OGB.20minus_chr8_141501469_141501492 | 100 | 23 | 2.00E−08 |
| 1129_Chr2_99927960_99928168 | Seq_GeCjCas9.OGB.20minus_chr11_84152405_84152428 | 100 | 23 | 3.00E−08 |
| 1129_Chr2_99927960_99928168 | Seq_GeCjCas9.OGB.20minus_chr11_84152405_84152428 | 95.65 | 23 | 8.00E−06 |
| 1129_Chr2_99927960_99928168 | Seq_GeCjCas9.OGB.20minus_chr1_102866197_102866220 | 95.65 | 23 | 8.00E−06 |
| 115_Chr1_24079090_24079202 | Seq_GeCjCas9.OGB.20minus_chr17_4861104_4861127 | 100 | 23 | 2.00E−08 |
| 1471_Chr2_219072954_219073050 | Seq_GeCjCas9.OGB.20minus_chr17_4861104_4861127 | 95.65 | 23 | 4.00E−06 |
| 1513_Chr2_232501114_232501223 | Seq_GeCjCas9.OGB.20minus_chr4_25224538_25224561 | 95.65 | 23 | 5.00E−06 |
| 1611_Chr3_14667912_14668025 | Seq_GeCjCas9.OGB.20minus_chr10_33287520_33287543 | 95.65 | 23 | 5.00E−06 |
| 1672_Chr3_32692995_32693124 | Seq_GeCjCas9.OGB.20minus_chr4_25224538_25224561 | 95.65 | 23 | 6.00E−06 |
| 1711_Chr3_45159251_45159353 | Seq_GeCjCas9.OGB.20minus_chr11_77993541_77993564 | 95.65 | 23 | 4.00E−06 |
| 1717_Chr3_47430996_47431114 | Seq_GeCjCas9.OGB.20minus_chr17_4861104_4861127 | 95.65 | 23 | 5.00E−06 |
| 183_Chr1_43651312_43651417 | Seq_GeCjCas9.OGB.20minus_chr5_31469254_31469277 | 95.65 | 23 | 4.00E−06 |
| 183_Chr1_43651312_43651417 | Seq_GeCjCas9.OGB.20minus_chr2_19125299_19125322 | 95.65 | 23 | 4.00E−06 |
| 183_Chr1_43651312_43651417 | Seq_GeCjCas9.OGB.20minus_chr12_633017_633040 | 95.65 | 23 | 4.00E−06 |
| 2188_Chr3_195844365_195844469 | Seq_GeCjCas9.OGB.20minus_chr9_37955762_37955785 | 95.65 | 23 | 4.00E−06 |
| 2188_Chr3_195844365_195844469 | Seq_GeCjCas9.OGB.20minus_chr1_18949543_18949566 | 95.65 | 23 | 4.00E−06 |
| 2227_Chr4_1708589_1708850 | Seq_GeCjCas9.OGB.20minus_chr17_4861104_4861127 | 100 | 23 | 4.00E−08 |
| 2227_Chr4_1708589_1708850 | Seq_GeCjCas9.OGB.20minus_chr17_4861104_4861127 | 100 | 23 | 4.00E−08 |
| 2231_Chr4_3335028_3335150 | Seq_GeCjCas9.OGB.20minus_chr2_61396269_61396292 | 95.65 | 23 | 5.00E−06 |
| 2383_Chr4_41129764_41129911 | Seq_GeCjCas9.OGB.20minus_chr5_31469254_31469277 | 100 | 23 | 3.00E−08 |
| 2383_Chr4_41129764_41129911 | Seq_GeCjCas9.OGB.20minus_chr5_31469254_31469277 | 100 | 23 | 3.00E−08 |
| 2383_Chr4_41129764_41129911 | Seq_GeCjCas9.OGB.20minus_chr12_633017_633040 | 100 | 23 | 3.00E−08 |
| 2383_Chr4_41129764_41129911 | Seq_GeCjCas9.OGB.20minus_chr12_633017_633040 | 100 | 23 | 3.00E−08 |
| 2572_Chr4_103410365_103410448 | Seq_GeCjCas9.OGB.20minus_chr5_15414511_15414534 | 95.65 | 23 | 3.00E−06 |
| 2637_Chr4_123398735_123398832 | Seq_GeCjCas9.OGB.20minus_chr7_77577434_77577457 | 95.65 | 23 | 4.00E−06 |
| 2713_Chr4_148579989_148580100 | Seq_GeCjCas9.OGB.20minus_chr4_25224538_25224561 | 95.65 | 23 | 5.00E−06 |
| 2726_Chr4_155034720_155034834 | Seq_GeCjCas9.OGB.20minus_chr2_40911491_40911514 | 95.65 | 23 | 5.00E−06 |
| 2726_Chr4_155034720_155034834 | Seq_GeCjCas9.OGB.20minus_chr2_40911491_40911514 | 95.65 | 23 | 5.00E−06 |
| 2765_Chr4_165781005_165781116 | Seq_GeCjCas9.OGB.20minus_chr17_4861104_4861127 | 100 | 23 | 2.00E−08 |
| 2835_Chr4_179859782_179859909 | Seq_GeCjCas9.OGB.20minus_chr17_4861104_4861127 | 95.65 | 23 | 5.00E−06 |
| 3095_Chr5_40817519_40817633 | Seq_GeCjCas9.OGB.20minus_chr4_25224538_25224561 | 95.65 | 23 | 5.00E−06 |
| 3352_Chr5_134057992_134058089 | Seq_GeCjCas9.OGB.20minus_chr5_31469254_31469277 | 100 | 23 | 2.00E−08 |
| 3352_Chr5_134057992_134058089 | Seq_GeCjCas9.OGB.20minus_chr12_633017_633040 | 100 | 23 | 2.00E−08 |
| 3465_Chr5_175785738_175785838 | Seq_GeCjCas9.OGB.20minus_chr17_4861104_4861127 | 95.65 | 23 | 4.00E−06 |
| 3595_Chr6_28955550_28955804 | Seq_GeCjCas9.OGB.20minus_chr17_4861104_4861127 | 100 | 23 | 4.00E−08 |
| 3648_Chr6_42994566_42994649 | Seq_GeCjCas9.OGB.20minus_chr22_20325962_20325985 | 95.65 | 23 | 3.00E−06 |
| 4085_Chr7_1117087_1117189 | Seq_GeCjCas9.OGB.20minus_chr14_38384381_38384404 | 95.65 | 23 | 4.00E−06 |
| 4122_Chr7_5841079_5841203 | Seq_GeCjCas9.OGB.20minus_chr10_84373887_84373910 | 95.65 | 23 | 5.00E−06 |
| 5328_Chr9_39478934_39479041 | Seq_GeCjCas9.OGB.20minus_chr17_4861104_4861127 | 100 | 23 | 2.00E−08 |
| 5328_Chr9_39478934_39479041 | Seq_GeCjCas9.OGB.20minus_chr22_20325962_20325985 | 95.65 | 23 | 5.00E−06 |
| 5328_Chr9_40806509_40806616 | Seq_GeCjCas9.OGB.20minus_chr17_4861104_4861127 | 100 | 23 | 2.00E−08 |
| 5328_Chr9_40806509_40806616 | Seq_GeCjCas9.OGB.20minus_chr22_20325962_20325985 | 95.65 | 23 | 5.00E−06 |
| 5328_Chr9_41623952_41624059 | Seq_GeCjCas9.OGB.20minus_chr17_4861104_4861127 | 100 | 23 | 2.00E−08 |
| 5328_Chr9_41623952_41624059 | Seq_GeCjCas9.OGB.20minus_chr22_20325962_20325985 | 95.65 | 23 | 5.00E−06 |
| 5416_Chr9_97060762_97060846 | Seq_GeCjCas9.OGB.20minus_chr5_31469254_31469277 | 95.65 | 23 | 3.00E−06 |
| 5416_Chr9_97060762_97060846 | Seq_GeCjCas9.OGB.20minus_chr12_633017_633040 | 95.65 | 23 | 3.00E−06 |
| 5517_Chr9_131430500_131430610 | Seq_GeCjCas9.OGB.20minus_chr8_141501469_141501492 | 95.65 | 23 | 5.00E−06 |
| 5526_Chr9_134170222_134170294 | Seq_GeCjCas9.OGB.20minus_chr1_150539650_150539673 | 95.65 | 23 | 3.00E−06 |
| 5528_Chr9_134330219_134330339 | Seq_GeCjCas9.OGB.20minus_chr1_237600585_237600608 | 95.65 | 23 | 5.00E−06 |
| 5690_Chr10_25063890_25063983 | Seq_GeCjCas9.OGB.20minus_chr14_105785516_105785539 | 95.65 | 23 | 4.00E−06 |
| 5732_Chr10_35154125_35154258 | Seq_GeCjCas9.OGB.20minus_chr11_77993541_77993564 | 95.65 | 23 | 6.00E−06 |
| 5927_Chr10_99470981_99471218 | Seq_GeCjCas9.OGB.20minus_chr5_31469254_31469277 | 100 | 23 | 4.00E−08 |
| 5927_Chr10_99470981_99471218 | Seq_GeCjCas9.OGB.20minus_chr12_633017_633040 | 100 | 23 | 4.00E−08 |
| 6224_Chr11_48839300_48839406 | Seq_GeCjCas9.OGB.20minus_chr16_35222615_35222638 | 95.65 | 23 | 4.00E−06 |
| 6224_Chr11_48839300_48839406 | Seq_GeCjCas9.OGB.20minus_chr10_42400588_42400611 | 95.65 | 23 | 4.00E−06 |
| 6224_Chr11_48839300_48839406 | Seq_GeCjCas9.OGB.20minus_chr10_42400586_42400609 | 95.65 | 23 | 4.00E−06 |
| 6251_Chr11_62746325_62746440 | Seq_GeCjCas9.OGB.20minus_chr10_84373887_84373910 | 95.65 | 23 | 5.00E−06 |
| 6398_Chr11_106194298_106194386 | Seq_GeCjCas9.OGB.20minus_chr10_30203146_30203169 | 95.65 | 23 | 4.00E−06 |
| 6430_Chr11_116672942_116673037 | Seq_GeCjCas9.OGB.20minus_chr17_4861104_4861127 | 100 | 23 | 2.00E−08 |
| 6430_Chr11_116672942_116673037 | Seq_GeCjCas9.OGB.20minus_chr17_4861104_4861127 | 100 | 23 | 2.00E−08 |
| 6549_Chr12_19578356_19578461 | Seq_GeCjCas9.OGB.20minus_chr17_4861104_4861127 | 100 | 23 | 2.00E−08 |
| 6844_Chr12_112837286_112837395 | Seq_GeCjCas9.OGB.20minus_chr5_31469254_31469277 | 95.65 | 23 | 5.00E−06 |
| 6844_Chr12_112837286_112837395 | Seq_GeCjCas9.OGB.20minus_chr12_633017_633040 | 95.65 | 23 | 5.00E−06 |
| 7369_Chr14_37751700_37751831 | Seq_GeCjCas9.OGB.20minus_chr5_31469254_31469277 | 95.65 | 23 | 6.00E−06 |
| 7369_Chr14_37751700_37751831 | Seq_GeCjCas9.OGB.20minus_chr12_633017_633040 | 95.65 | 23 | 6.00E−06 |
| 7565_Chr14_102517614_102517709 | Seq_GeCjCas9.OGB.20minus_chr17_4861104_4861127 | 100 | 23 | 2.00E−08 |
| 7567_Chr14_102955734_102955856 | Seq_GeCjCas9.OGB.20minus_chr5_31469254_31469277 | 95.65 | 23 | 5.00E−06 |
| 7567_Chr14_102955734_102955856 | Seq_GeCjCas9.OGB.20minus_chr12_633017_633040 | 95.65 | 23 | 5.00E−06 |
| 7645_Chr15_33901557_33901681 | Seq_GeCjCas9.OGB.20minus_chr17_4861104_4861127 | 100 | 23 | 2.00E−08 |
| 7677_Chr15_44758402_44758502 | Seq_GeCjCas9.OGB.20minus_chr8_141501469_141501492 | 95.65 | 23 | 4.00E−06 |
| 7839_Chr16_423673_423876 | Seq_GeCjCas9.OGB.20minus_chr10_70837929_70837952 | 95.65 | 23 | 8.00E−06 |
| 7903_Chr16_14391749_14391872 | Seq_GeCjCas9.OGB.20minus_chr17_4861104_4861127 | 100 | 23 | 2.00E−08 |
| 7903_Chr16_14391749_14391872 | Seq_GeCjCas9.OGB.20minus_chr17_4861104_4861127 | 95.65 | 23 | 5.00E−06 |
| 7936_Chr16_21584700_21584808 | Seq_GeCjCas9.OGB.20minus_chr4_25224538_25224561 | 95.65 | 23 | 5.00E−06 |
| 8016_Chr16_69825675_69825753 | Seq_GeCjCas9.OGB.20minus_chr6_57173198_57173221 | 95.65 | 23 | 3.00E−06 |
| 8022_Chr16_72062630_72062754 | Seq_GeCjCas9.OGB.20minus_chr4_25224538_25224561 | 95.65 | 23 | 5.00E−06 |
| 8176_Chr17_9045196_9045306 | Seq_GeCjCas9.OGB.20minus_chr5_31469254_31469277 | 100 | 23 | 2.00E−08 |
| 8176_Chr17_9045196_9045306 | Seq_GeCjCas9.OGB.20minus_chr12_633017_633040 | 100 | 23 | 2.00E−08 |
| 8269_Chr17_46994135_46994227 | Seq_GeCjCas9.OGB.20minus_chr5_31469254_31469277 | 95.65 | 23 | 4.00E−06 |
| 8269_Chr17_46994135_46994227 | Seq_GeCjCas9.OGB.20minus_chr2_19125299_19125322 | 95.65 | 23 | 4.00E−06 |
| 8269_Chr17_46994135_46994227 | Seq_GeCjCas9.OGB.20minus_chr12_633017_633040 | 95.65 | 23 | 4.00E−06 |
| 8311_Chr17_58019456_58019571 | Seq_GeCjCas9.OGB.20minus_chr14_105785516_105785539 | 95.65 | 23 | 5.00E−06 |
| 8376_Chr17_76278190_76278306 | Seq_GeCjCas9.OGB.20minus_chr4_25224538_25224561 | 95.65 | 23 | 5.00E−06 |
| 8378_Chr17_76790747_76790854 | Seq_GeCjCas9.OGB.20minus_chr4_25224538_25224561 | 95.65 | 23 | 5.00E−06 |
| 8384_Chr17_77719715_77719791 | Seq_GeCjCas9.OGB.20minus_chr5_31469254_31469277 | 95.65 | 23 | 3.00E−06 |
| 8384_Chr17_77719715_77719791 | Seq_GeCjCas9.OGB.20minus_chr12_633017_633040 | 95.65 | 23 | 3.00E−06 |
| 8594_Chr18_67847548_67847684 | Seq_GeCjCas9.OGB.20minus_chr5_31469254_31469277 | 95.65 | 23 | 6.00E−06 |
| 8594_Chr18_67847548_67847684 | Seq_GeCjCas9.OGB.20minus_chr2_19125299_19125322 | 95.65 | 23 | 6.00E−06 |
| 8594_Chr18_67847548_67847684 | Seq_GeCjCas9.OGB.20minus_chr12_633017_633040 | 95.65 | 23 | 6.00E−06 |
| 8686_Chr19_2837128_2837261 | Seq_GeCjCas9.OGB.20minus_chr5_31469254_31469277 | 100 | 23 | 2.00E−08 |
| 8686_Chr19_2837128_2837261 | Seq_GeCjCas9.OGB.20minus_chr12_633017_633040 | 100 | 23 | 2.00E−08 |
| 8773_Chr19_24585850_24585981 | Seq_GeCjCas9.OGB.20minus_chr7_61968701_61968724 | 95.65 | 23 | 6.00E−06 |
| 8773_Chr19_24585850_24585981 | Seq_GeCjCas9.OGB.20minus_chr1_121485280_121485303 | 95.65 | 23 | 6.00E−06 |
| 8773_Chr19_24585850_24585981 | Seq_GeCjCas9.OGB.20minus_chr1_121484767_121484790 | 95.65 | 23 | 6.00E−06 |
| 8991_Chr20_31299717_31299831 | Seq_GeCjCas9.OGB.20minus_chr17_4861104_4861127 | 95.65 | 23 | 5.00E−06 |
| 9041_Chr20_47791131_47791244 | Seq_GeCjCas9.OGB.20minus_chr17_4861104_4861127 | 100 | 23 | 2.00E−08 |
| 9041_Chr20_47791131_47791244 | Seq_GeCjCas9.OGB.20minus_chr17_4861104_4861127 | 95.65 | 23 | 5.00E−06 |
| 9345_Chr22_29847209_29847300 | Seq_GeCjCas9.OGB.20minus_chr17_4861104_4861127 | 100 | 23 | 2.00E−08 |
| 9345_Chr22_29847209_29847300 | Seq_GeCjCas9.OGB.20minus_chr17_4861104_4861127 | 95.65 | 23 | 4.00E−06 |
| 9352_Chr22_31657195_31657308 | Seq_GeCjCas9.OGB.20minus_chr17_4861104_4861127 | 100 | 23 | 2.00E−08 |
| 9378_Chr22_39847505_39847612 | Seq_GeCjCas9.OGB.20minus_chr17_4861104_4861127 | 100 | 23 | 2.00E−08 |
| 9385_Chr22_41341585_41341694 | Seq_GeCjCas9.OGB.20minus_chr2_61396269_61396292 | 95.65 | 23 | 5.00E−06 |
| 9393_Chr22_42647977_42648072 | Seq_GeCjCas9.OGB.20minus_chr5_31469254_31469277 | 95.65 | 23 | 4.00E−06 |
| 9393_Chr22_42647977_42648072 | Seq_GeCjCas9.OGB.20minus_chr2_19125299_19125322 | 95.65 | 23 | 4.00E−06 |
| 9393_Chr22_42647977_42648072 | Seq_GeCjCas9.OGB.20minus_chr12_633017_633040 | 95.65 | 23 | 4.00E−06 |
| 1513_Chr2_232501114_232501223 | Seq_GeCjCas9.OGB.20minus_chr17_4861104_4861127 | 100 | 22 | 7.00E−08 |
| 1672_Chr3_32692995_32693124 | Seq_GeCjCas9.OGB.20minus_chr17_4861104_4861127 | 100 | 22 | 9.00E−08 |
| 1672_Chr3_32692995_32693124 | Seq_GeCjCas9.OGB.20minus_chr17_4861104_4861127 | 100 | 22 | 9.00E−08 |
| 2381_Chr4_41066259_41066323 | Seq_GeCjCas9.OGB.20minus_chr9_91913001_91913024 | 95.45 | 22 | 1.00E−05 |
| 2381_Chr4_41066259_41066323 | Seq_GeCjCas9.OGB.20minus_chr7_146571708_146571731 | 95.45 | 22 | 1.00E−05 |
| 2637_Chr4_123398735_123398832 | Seq_GeCjCas9.OGB.20minus_chr11_55391880_55391903 | 100 | 22 | 7.00E−08 |
| 2713_Chr4_148579989_148580100 | Seq_GeCjCas9.OGB.20minus_chr17_4861104_4861127 | 100 | 22 | 8.00E−08 |
| 3081_Chr5_37367004_37367113 | Seq_GeCjCas9.OGB.20minus_chr2_61396269_61396292 | 100 | 22 | 7.00E−08 |
| 3095_Chr5_40817519_40817633 | Seq_GeCjCas9.OGB.20minus_chr17_4861104_4861127 | 100 | 22 | 8.00E−08 |
| 4085_Chr7_1117087_1117189 | Seq_GeCjCas9.OGB.20minus_chr8_141501469_141501492 | 100 | 22 | 7.00E−08 |
| 5931_Chr10_100924520_100924608 | Seq_GeCjCas9.OGB.20minus_chr17_4861104_4861127 | 100 | 22 | 6.00E−08 |
| 6215_Chr11_45570011_45570123 | Seq_GeCjCas9.OGB.20minus_chr17_4861104_4861127 | 100 | 22 | 8.00E−08 |
| 6222_Chr11_48829963_48830063 | Seq_GeCjCas9.OGB.20minus_chr17_22261284_22261307 | 100 | 22 | 7.00E−08 |
| 6251_Chr11_62746325_62746440 | Seq_GeCjCas9.OGB.20minus_chr2_61396269_61396292 | 100 | 22 | 8.00E−08 |
| 6871_Chr12_122174321_122174450 | Seq_GeCjCas9.OGB.20minus_chr2_61396269_61396292 | 100 | 22 | 9.00E−08 |
| 7342_Chr14_28266246_28266360 | Seq_GeCjCas9.OGB.20minus_chr17_4861104_4861127 | 100 | 22 | 8.00E−08 |
| 7936_Chr16_21584700_21584808 | Seq_GeCjCas9.OGB.20minus_chr17_4861104_4861127 | 100 | 22 | 7.00E−08 |
| 8012_Chr16_67844992_67845092 | Seq_GeCjCas9.OGB.20minus_chr8_141501469_141501492 | 100 | 22 | 7.00E−08 |
| 8022_Chr16_72062630_72062754 | Seq_GeCjCas9.OGB.20minus_chr17_4861104_4861127 | 100 | 22 | 9.00E−08 |
| 8204_Chr17_19149239_19149347 | Seq_GeCjCas9.OGB.20minus_chr22_24409925_24409948 | 100 | 22 | 7.00E−08 |
| 8218_Chr17_29052153_29052275 | Seq_GeCjCas9.OGB.20minus_chr8_141501469_141501492 | 100 | 22 | 8.00E−08 |
| 8376_Chr17_76278190_76278306 | Seq_GeCjCas9.OGB.20minus_chr17_4861104_4861127 | 100 | 22 | 8.00E−08 |
| 8378_Chr17_76790747_76790854 | Seq_GeCjCas9.OGB.20minus_chr17_4861104_4861127 | 100 | 22 | 7.00E−08 |
| 8773_Chr19_24585850_24585981 | Seq_GeCjCas9.OGB.20minus_chr7_61968711_61968734 | 100 | 22 | 9.00E−08 |
| 9385_Chr22_41341585_41341694 | Seq_GeCjCas9.OGB.20minus_chr8_141501469_141501492 | 100 | 22 | 7.00E−08 |
| 9397_Chr22_43417966_43418096 | Seq_GeCjCas9.OGB.20minus_chr17_4861104_4861127 | 100 | 22 | 9.00E−08 |
| 9397_Chr22_43417966_43418096 | Seq_GeCjCas9.OGB.20minus_chr17_4861104_4861127 | 100 | 22 | 9.00E−08 |

^{A}first the SEQ ID NO is shown and is concatenated with the chromosome position were this human CRISPR receded in the reference human Hg19 genome;
^{B}first mentions that the DSB break site is found at the exact same position during C. jejuni infection and plasmid transfection of U2OS cells and is CjCas9 dependent. This ID is concatenated to the genome position were this break occurred;
^{C}shows the match in percentage of the human CRISPR sequence with the break site;
^{D}shows the number of nucleotides in base pairs of the human CRISPRs that exactly match with the break site;
^{E}shows the significance of CRISPR match with the break site.

### Example 6

### Human CRISPR Transcription is Influenced by (a)biotic Compounds Including Medicine

The more than 35.000 human gene targets as defined in Example 1 that are under transcription regulation control of the human CRISPRs were uploaded into the Ingenuity pathway analysis (IPA) software and established important roles in a wide variety of cellular and tissue functions and disease, which are visualized in a Top list presented in (Table 3) and a more detailed list presented in (Table 4).

TABLE 3
Top list on the role of the human CRISPRs and their gene targets
in disease, development and cellular functions.
Top Diseases and Functions

Nervous System Development and Function, Tissue Morphology, Embryonic Development
Cancer, Gastrointestinal Disease, Organismal Injury and Abnormalities
Developmental Disorder, Hereditary Disorder, Neurological Disease
Cell Signaling, Cell Death and Survival, Neurological Disease
Connective Tissue Disorders, Developmental Disorder, Skeletal and Muscular Disorders
Cell-To-Cell Signaling and Interaction, Cell Signaling, Vitamin and Mineral Metabolism
Drug Metabolism, Endocrine System Development and Function, Lipid Metabolism
Immunological Disease, Connective Tissue Disorders, Cell-To-Cell Signaling and Interaction
Cellular Compromise, Cellular Function and Maintenance, Connective Tissue Disorders
Lipid Metabolism, Small Molecule Biochemistry, Developmental Disorder
Neurological Disease, Organismal Injury and Abnormalities, Nervous System Development and Function
Cellular Development, Cellular Growth and Proliferation, Embryonic Development
Humoral Immune Response, Lymphoid Tissue Structure and Development, Cell-mediated Immune Response
Cellular Development, Cancer, Cell Cycle
Cellular Assembly and Organization, Cellular Movement, Gene Expression
Hematological Disease, Cell Death and Survival, Cellular Assembly and Organization
Gastrointestinal Disease, Organismal Injury and Abnormalities, Carbohydrate Metabolism
Cellular Assembly and Organization, Cellular Function and Maintenance, Developmental Disorder
Cellular Compromise, Developmental Disorder, Organismal Injury and Abnormalities
Lipid Metabolism, Small Molecule Biochemistry, Cell-To-Cell Signaling and Interaction
Hereditary Disorder, Neurological Disease, Organismal Injury and Abnormalities
Organismal Injury and Abnormalities, Reproductive System Disease, Cellular Function and Maintenance
Cellular Development, Cellular Growth and Proliferation, Nervous System Development and Function
Cell-To-Cell Signaling and Interaction, Cellular Assembly and Organization, Cellular Development
Developmental Disorder, Hereditary Disorder, Neurological Disease
The ±35.000 human genes that harbored a significant BLAST hit with the human CRISPRs were uploaded in ingenuity pathway analyses and provided a Top list of diseases, development and other functions in which these genes are involved

TABLE 4
Detailed list on the role of the human CRISPRs and their gene targets in disease, development and cellular functions.

| Nr | | Diseases or Functions | p- |
|---|---|---|---|
| ID^{A} | Categories^{B} | Annotation^{B} | Value^{C} |
| 3 | Gastrointestinal Disease, Hepatic System Disease, Organismal Injury and | liver lesion | 6.78E−23 |
| | Abnormalities | | |
| 4 | Cancer, Gastrointestinal Disease, Hepatic System Disease, Organismal Injury and | hepatobiliary system | 2.45E−22 |
| | Abnormalities | cancer | |
| 5 | Cancer, Gastrointestinal Disease, Hepatic System Disease, Organismal Injury and | liver tumor | 5.36E−22 |
| | Abnormalities | | |
| 6 | Cancer, Gastrointestinal Disease, Hepatic System Disease, Organismal Injury and | liver cancer | 6.80E−22 |
| | Abnormalities | | |
| 7 | Cancer, Organismal Injury and Abnormalities, Reproductive System Disease | endometrial carcinoma | 5.00E−21 |
| 8 | Cancer, Gastrointestinal Disease, Organismal Injury and Abnormalities | digestive organ tumor | 6.95E−21 |
| 9 | Cancer, Gastrointestinal Disease, Organismal Injury and Abnormalities | digestive system cancer | 1.03E−20 |
| 10 | Cancer, Dermatological Diseases and Conditions, Organismal Injury and | malignant cutaneous | 3.84E−20 |
| | Abnormalities | melanoma cancer | |
| 11 | Cancer, Organismal Injury and Abnormalities, Reproductive System Disease | adenocarcinoma in | 3.96E−20 |
| | | endometrium | |
| 12 | Cancer, Organismal Injury and Abnormalities, Reproductive System Disease | uterine carcinoma | 4.68E−20 |
| 13 | Cancer, Organismal Injury and Abnormalities | malignant solid tumor | 6.91E−20 |
| 14 | Cancer, Organismal Injury and Abnormalities | melanoma | 1.07E−19 |
| 15 | Cancer, Gastrointestinal Disease, Organismal Injury and Abnormalities | gastrointestinal tract | 1.37E−19 |
| | | cancer | |
| 16 | Cancer, Dermatological Diseases and Conditions, Organismal Injury and | skin cancer | 1.94E−19 |
| | Abnormalities | | |
| 17 | Cancer, Gastrointestinal Disease, Organismal Injury and Abnormalities | Gastrointestinal Tract | 2.60E−19 |
| | | Cancer and Tumors | |
| 18 | Cancer, Dermatological Diseases and Conditions, Organismal Injury and | skin tumor | 2.78E−19 |
| | Abnormalities | | |
| 19 | Cancer, Organismal Injury and Abnormalities | abdominal neoplasm | 3.45E−19 |
| 20 | Cancer, Organismal Injury and Abnormalities | cancer | 8.19E−19 |
| 21 | Cancer, Organismal Injury and Abnormalities, Reproductive System Disease | endometrial cancer | 1.09E−18 |
| 22 | Cancer, Gastrointestinal Disease, Organismal Injury and Abnormalities | intestinal tumor | 1.15E−18 |
| 23 | Cancer, Gastrointestinal Disease, Organismal Injury and Abnormalities | large intestine neoplasm | 1.70E−18 |
| 24 | Cancer, Organismal Injury and Abnormalities | abdominal cancer | 1.79E−18 |
| 25 | Cancer, Organismal Injury and Abnormalities | neoplasia of epithelial | 1.98E−18 |
| | | tissue | |
| 26 | Cancer, Gastrointestinal Disease, Organismal Injury and Abnormalities | intestinal cancer | 2.61E−18 |
| 27 | Cancer, Gastrointestinal Disease, Organismal Injury and Abnormalities | malignant neoplasm of | 4.70E−18 |
| | | large intestine | |
| 28 | Cancer, Organismal Injury and Abnormalities | epithelial cancer | 4.86E−18 |
| 29 | Cancer, Organismal Injury and Abnormalities | endometrioid carcinoma | 4.99E−18 |
| 30 | Cancer, Organismal Injury and Abnormalities | tumorigenesis of tissue | 1.22E−17 |
| 31 | Cancer, Organismal Injury and Abnormalities, Reproductive System Disease | uterine cancer | 2.31E−17 |
| 32 | Cancer, Organismal Injury and Abnormalities | abdominal | 2.67E−17 |
| | | adenocarcinoma | |
| 33 | Cancer, Organismal Injury and Abnormalities, Reproductive System Disease | female genital tract | 3.03E−17 |
| | | adenocarcinoma | |
| 34 | Cancer, Organismal Injury and Abnormalities | adenocarcinoma | 3.47E−17 |
| 35 | Cancer, Organismal Injury and Abnormalities, Reproductive System Disease | uterine tumor | 4.42E−17 |
| 36 | Cancer, Organismal Injury and Abnormalities | abdominal carcinoma | 7.09E−17 |
| 37 | Cancer, Organismal Injury and Abnormalities, Reproductive System Disease | genital tract cancer | 5.23E−16 |
| 38 | Cancer, Organismal Injury and Abnormalities | genitourinary carcinoma | 7.79E−16 |
| 39 | Cancer, Organismal Injury and Abnormalities, Reproductive System Disease | female genital tract | 3.83E−15 |
| | | cancer | |
| 40 | Cancer, Organismal Injury and Abnormalities, Reproductive System Disease | genital tumor | 7.72E−15 |
| 41 | Cancer, Organismal Injury and Abnormalities, Reproductive System Disease | tumorigenesis of genital | 1.66E−14 |
| | | organ | |
| 42 | Cancer, Organismal Injury and Abnormalities | pelvic cancer | 2.34E−14 |
| 43 | Cancer, Organismal Injury and Abnormalities, Reproductive System Disease | female genital neoplasm | 2.51E−14 |
| 44 | Cancer, Organismal Injury and Abnormalities, Reproductive System Disease | tumorigenesis of | 5.21E−14 |
| | | reproductive tract | |
| 45 | Cancer, Organismal Injury and Abnormalities | cancer of secretory | 1.19E−13 |
| | | structure | |
| 46 | Cancer, Organismal Injury and Abnormalities | urogenital cancer | 1.22E−13 |
| 47 | Cancer, Gastrointestinal Disease, Organismal Injury and Abnormalities | gastrointestinal | 2.08E−13 |
| | | carcinoma | |
| 48 | Cancer, Gastrointestinal Disease, Organismal Injury and Abnormalities | gastrointestinal | 2.52E−13 |
| | | adenocarcinoma | |
| 49 | Cancer, Gastrointestinal Disease, Organismal Injury and Abnormalities | gastroesophageal | 3.42E−13 |
| | | adenocarcinoma | |
| 50 | Cancer, Gastrointestinal Disease, Organismal Injury and Abnormalities | upper gastrointestinal | 7.69E−13 |
| | | tract cancer | |
| 51 | Cancer, Dermatological Diseases and Conditions, Organismal Injury and | squamous cell skin cancer | 8.03E−13 |
| | Abnormalities | | |
| 52 | Cancer, Gastrointestinal Disease, Organismal Injury and Abnormalities | gastroesophageal cancer | 1.44E−12 |
| 53 | Cancer, Organismal Injury and Abnormalities | ductal carcinoma | 1.73E−12 |
| 54 | Cancer, Gastrointestinal Disease, Organismal Injury and Abnormalities | gastroesophageal | 2.00E−12 |
| | | carcinoma | |
| 55 | Cancer, Endocrine System Disorders, Gastrointestinal Disease, Organismal Injury | ductal pancreatic | 2.49E−12 |
| | and Abnormalities | carcinoma | |
| 56 | Cancer, Endocrine System Disorders, Gastrointestinal Disease, Organismal Injury | pancreatic tumor | 3.40E−12 |
| | and Abnormalities | | |
| 57 | Cancer, Dermatological Diseases and Conditions, Organismal Injury and | skin carcinoma | 4.64E−12 |
| | Abnormalities | | |
| 58 | Gastrointestinal Disease | gastric lesion | 5.86E−12 |
| 59 | Cancer, Gastrointestinal Disease, Organismal Injury and Abnormalities | gastric cancer | 7.02E−12 |
| 60 | Cancer, Organismal Injury and Abnormalities, Respiratory Disease | lung tumor | 2.54E−11 |
| 61 | Cancer, Organismal Injury and Abnormalities | thoracic neoplasm | 2.55E−11 |
| 62 | Cancer, Organismal Injury and Abnormalities | breast or colorectal cancer | 3.13E−11 |
| 63 | Cancer, Organismal Injury and Abnormalities, Respiratory Disease | lung cancer | 5.69E−11 |
| 64 | Cancer, Endocrine System Disorders, Gastrointestinal Disease, Organismal Injury | pancreatic cancer | 5.82E−11 |
| | and Abnormalities | | |
| 65 | Inflammatory Disease | chronic inflammatory | 1.30E−10 |
| | | disorder | |
| 66 | Cancer, Organismal Injury and Abnormalities, Respiratory Disease | non-small cell lung cancer | 1.53E−10 |
| 67 | Cancer, Gastrointestinal Disease, Organismal Injury and Abnormalities | gastric adenocarcinoma | 1.63E−10 |
| 68 | Cancer, Organismal Injury and Abnormalities | malignant connective or | 2.65E−10 |
| | | soft tissue neoplasm | |
| 69 | Cancer, Gastrointestinal Disease, Organismal Injury and Abnormalities | colorectal carcinoma | 2.73E−10 |
| 70 | Cancer, Gastrointestinal Disease, Organismal Injury and Abnormalities | gastric carcinoma | 3.08E−10 |
| 71 | Cancer, Organismal Injury and Abnormalities, Respiratory Disease | carcinoma in lung | 3.08E−10 |
| 72 | Cancer, Gastrointestinal Disease, Organismal Injury and Abnormalities | colon carcinoma | 3.97E−10 |
| 73 | Cancer, Organismal Injury and Abnormalities | connective or soft tissue | 4.52E−10 |
| | | tumor | |
| 74 | Cancer, Gastrointestinal Disease, Organismal Injury and Abnormalities | intestinal adenocarcinoma | 5.01E−10 |
| 75 | Cancer, Organismal Injury and Abnormalities, Skeletal and Muscular Disorders | embryonal | 5.36E−10 |
| | | rhabdomyosarcoma | |
| 76 | Cancer, Gastrointestinal Disease, Organismal Injury and Abnormalities | colon adenocarcinoma | 8.20E−10 |
| 77 | Cancer, Gastrointestinal Disease, Organismal Injury and Abnormalities | colorectal adenocarcinoma | 9.26E−10 |
| 78 | Cancer, Organismal Injury and Abnormalities | connective tissue tumor | 9.71E−10 |
| 79 | Cancer, Organismal Injury and Abnormalities | sarcoma | 1.13E−09 |
| 80 | Cancer, Gastrointestinal Disease, Organismal Injury and Abnormalities | colon tumor | 2.01E−09 |
| 81 | Cancer, Gastrointestinal Disease, Organismal Injury and Abnormalities | colorectal neoplasia | 2.08E−09 |
| 82 | Behavior | behavior | 2.19E−09 |
| 83 | Organismal Development | abnormal morphology of | 2.23E−09 |
| | | head | |
| 84 | Cancer, Gastrointestinal Disease, Organismal Injury and Abnormalities | colorectal cancer | 2.28E−09 |
| 85 | Cell Morphology, Cellular Assembly and Organization, Cellular Function and | formation of cellular | 2.32E−09 |
| | Maintenance | protrusions | |
| 86 | Endocrine System Disorders, Gastrointestinal Disease, Metabolic Disease | diabetes mellitus | 2.54E−09 |
| 87 | Cancer, Organismal Injury and Abnormalities, Skeletal and Muscular Disorders | muscle tumor | 3.04E−09 |
| 88 | Immunological Disease | systemic autoimmune | 3.34E−09 |
| | | syndrome | |
| 89 | Organismal Development | morphology of head | 3.62E−09 |
| 90 | Cancer, Organismal Injury and Abnormalities, Skeletal and Muscular Disorders | myosarcoma | 3.98E−09 |
| 91 | Cancer, Gastrointestinal Disease, Organismal Injury and Abnormalities | colon cancer | 4.47E−09 |
| 92 | Cellular Growth and Proliferation, Tissue Development | generation of cells | 4.93E−09 |
| 93 | Cancer, Organismal Injury and Abnormalities, Skeletal and Muscular Disorders | rhabdomyosarcoma | 5.19E−09 |
| 94 | Psychological Disorders | Mood Disorders | 6.48E−09 |
| 95 | Nervous System Development and Function | morphology of nervous | 1.08E−08 |
| | | system | |
| 96 | Nervous System Development and Function | abnormal morphology of | 1.27E−08 |
| | | nervous system | |
| 97 | Cancer, Organismal Injury and Abnormalities | breast or ovarian | 1.41E−08 |
| | | carcinoma | |
| 98 | Hereditary Disorder, Organismal Injury and Abnormalities | autosomal recessive | 1.47E−08 |
| | | disease | |
| 99 | Cellular Development, Cellular Growth and Proliferation, Nervous System | development of neurons | 2.39E−08 |
| | Development and Function, Tissue Development | | |
| 100 | Organismal Survival | organismal death | 2.70E−08 |
| 101 | Cancer, Organismal Injury and Abnormalities | liquid tumor | 2.72E−08 |
| 102 | Nervous System Development and Function | development of central | 2.96E−08 |
| | | nervous system | |
| 103 | Cancer, Organismal Injury and Abnormalities | malignant neoplasm of | 3.08E−08 |
| | | aerodigestive tract | |
| 104 | Metabolic Disease | glucose metabolism | 3.40E−08 |
| | | disorder | |
| 105 | Cancer, Organismal Injury and Abnormalities | cancer of cells | 4.39E−08 |
| 106 | Connective Tissue Disorders, Immunological Disease, Inflammatory Disease, | rheumatoid arthritis | 6.06E−08 |
| | Inflammatory Response, Skeletal and Muscular Disorders | | |
| 107 | Cancer, Organismal Injury and Abnormalities | soft tissue neoplasm | 7.16E−08 |
| 108 | Lipid Metabolism, Small Molecule Biochemistry | fatty acid metabolism | 8.61E−08 |
| 109 | Cancer, Organismal Injury and Abnormalities, Reproductive System Disease | breast or ovarian cancer | 1.05E−07 |
| 110 | Embryonic Development, Organismal Development | development of head | 1.12E−07 |
| 111 | Behavior | cognition | 1.31E−07 |
| 112 | Organismal Injury and Abnormalities | Organ Degeneration | 1.35E−07 |
| 113 | Cancer, Organismal Injury and Abnormalities | upper aerodigestive tract | 1.37E−07 |
| | | carcinoma | |
| 114 | Neurological Disease, Organismal Injury and Abnormalities | gliosis | 1.53E−07 |
| 115 | Embryonic Development, Organismal Development | development of body axis | 1.66E−07 |
| 116 | Cancer, Hematological Disease, Immunological Disease, Organismal Injury and | acute leukemia | 1.87E−07 |
| | Abnormalities | | |
| 117 | Neurological Disease, Psychological Disorders | bipolar disorder | 1.92E−07 |
| 118 | Cardiovascular Disease | peripheral vascular | 1.98E−07 |
| | | disease | |
| 119 | Behavior | learning | 2.01E−07 |
| 120 | Cancer, Hematological Disease, Immunological Disease, Organismal Injury and | leukemia | 2.08E−07 |
| | Abnormalities | | |
| 121 | Cancer, Organismal Injury and Abnormalities | squamous-cell carcinoma | 2.15E−07 |
| 122 | Cellular Assembly and Organization | formation of cellular | 2.57E−07 |
| | | membrane | |
| 123 | Cell-To-Cell Signaling and Interaction, Cellular Assembly and Organization, | developmental process of | 2.76E−07 |
| | Cellular Development, Cellular Function and Maintenance, Cellular Growth and | synapse | |
| | Proliferation, Nervous System Development and Function, Tissue Development | | |
| 124 | Neurological Disease | Movement Disorders | 3.10E−07 |
| 125 | Cellular Assembly and Organization, Cellular Function and Maintenance | microtubule dynamics | 3.14E−07 |
| 126 | Neurological Disease | neurodegeneration of | 3.26E−07 |
| | | central nervous system | |
| 127 | Cancer, Gastrointestinal Disease, Organismal Injury and Abnormalities | esophageal | 3.60E−07 |
| | | adenocarcinoma | |
| 128 | Cellular Assembly and Organization, Cellular Function and Maintenance | organization of cytoplasm | 3.75E−07 |
| 129 | Cell Morphology | abnormal morphology of | 3.79E−07 |
| | | cells | |
| 130 | Cell-To-Cell Signaling and Interaction, Cellular Assembly and Organization, | assembly of intercellular | 4.67E−07 |
| | Cellular Function and Maintenance | junctions | |
| 131 | Nervous System Development and Function, Organ Morphology, Organismal | morphology of brain | 5.23E−07 |
| | Development | | |
| 132 | Cancer, Gastrointestinal Disease, Organismal Injury and Abnormalities | esophageal carcinoma | 5.41E−07 |
| 133 | Psychological Disorders | major affective disorder | 5.86E−07 |
| 134 | Connective Tissue Disorders, Skeletal and Muscular Disorders | arthropathy | 6.20E−07 |
| 135 | Nervous System Development and Function, Organ Morphology, Organismal | abnormal morphology of | 6.33E−07 |
| | Development | brain | |
| 136 | Cellular Assembly and Organization | formation of plasma | 7.44E−07 |
| | | membrane | |
| 137 | Cancer, Endocrine System Disorders, Organismal Injury and Abnormalities, | Ovarian Cancer and | 7.72E−07 |
| | Reproductive System Disease | Tumors | |
| 138 | Cancer, Organismal Injury and Abnormalities | Lymphoid Cancer and | 8.03E−07 |
| | | Tumors | |
| 139 | Cancer, Endocrine System Disorders, Organismal Injury and Abnormalities, | epithelial ovarian cancer | 9.85E−07 |
| | Reproductive System Disease | | |
| 140 | Cancer, Hematological Disease, Immunological Disease, Organismal Injury and | hematological neoplasia | 1.09E−06 |
| | Abnormalities | | |
| 141 | Cellular Assembly and Organization, Cellular Function and Maintenance | organization of | 1.41E−06 |
| | | cytoskeleton | |
| 142 | Cancer, Endocrine System Disorders, Organismal Injury and Abnormalities, | gonadal tumor | 1.45E−06 |
| | Reproductive System Disease | | |
| 143 | Embryonic Development, Nervous System Development and Function, Organ | formation of brain | 1.57E−06 |
| | Development, Organismal Development, Tissue Development | | |
| 144 | Embryonic Development, Organismal Development | development of body | 1.76E−06 |
| | | trunk | |
| 145 | Cancer, Gastrointestinal Disease, Organismal Injury and Abnormalities | esophageal cancer | 1.77E−06 |
| 146 | Molecular Transport | transport of molecule | 1.86E−06 |
| 147 | Neurological Disease, Organismal Injury and Abnormalities | neurodegeneration of | 1.86E−06 |
| | | brain | |
| 148 | Connective Tissue Disorders, Inflammatory Disease, Inflammatory Response, | inflammation of joint | 2.11E−06 |
| | Skeletal and Muscular Disorders | | |
| 149 | Neurological Disease, Organismal Injury and Abnormalities | reactive astrogliosis | 2.17E−06 |
| 150 | Organismal Injury and Abnormalities | discomfort | 2.60E−06 |
| 151 | Cancer, Endocrine System Disorders, Organismal Injury and Abnormalities, | ovarian cancer | 2.68E−06 |
| | Reproductive System Disease | | |
| 152 | Cancer, Hematological Disease, Immunological Disease, Organismal Injury and | myeloid neoplasm | 2.76E−06 |
| | Abnormalities | | |
| 153 | Psychological Disorders | depressive disorder | 2.87E−06 |
| 154 | Organismal Injury and Abnormalities, Renal and Urological Disease | renal impairment | 3.23E−06 |
| 155 | Organismal Injury and Abnormalities | intracranial hemorrhage | 3.31E−06 |
| 156 | Cancer, Hematological Disease, Immunological Disease, Organismal Injury and | lymphoid cancer | 3.35E−06 |
| | Abnormalities | | |
| 157 | Psychological Disorders | schizophrenia spectrum | 3.48E−06 |
| | | disorder | |
| 158 | Cancer, Cardiovascular Disease, Organismal Injury and Abnormalities | hemangioma | 3.63E−06 |
| 159 | Nervous System Development and Function, Tissue Morphology | quantity of neurons | 3.69E−06 |
| 160 | Cardiovascular Disease | Hypertension | 3.88E−06 |
| 161 | Developmental Disorder | multiple congenital | 4.01E−06 |
| | | anomalies | |
| 162 | Organismal Development | abnormal morphology of | 4.30E−06 |
| | | body cavity | |
| 163 | Cell-To-Cell Signaling and Interaction | cell-cell adhesion | 4.35E−06 |
| 164 | Cardiovascular Disease | occlusion of artery | 4.53E−06 |
| 165 | Cancer, Hematological Disease, Immunological Disease, Organismal Injury and | myeloproliferative | 5.15E−06 |
| | Abnormalities | disorder | |
| 166 | Cancer, Organismal Injury and Abnormalities, Respiratory Disease | squamous cell lung cancer | 6.13E−06 |
| 167 | Cardiovascular Disease, Organismal Injury and Abnormalities | coronary disease | 6.36E−06 |
| 168 | Organismal Development | abnormal morphology of | 6.60E−06 |
| | | thoracic cavity | |
| 169 | Cancer, Cardiovascular Disease, Organismal Injury and Abnormalities | capillary hemangioma | 7.36E−06 |
| 170 | Organismal Injury and Abnormalities, Renal and Urological Disease | failure of kidney | 8.03E−06 |
| 171 | Organismal Injury and Abnormalities, Renal and Urological Disease | benign renal mass | 8.08E−06 |
| 172 | Organismal Development | morphology of body cavity | 8.32E−06 |
| 173 | Cancer, Endocrine System Disorders, Organismal Injury and Abnormalities, | ovarian adenocarcinoma | 9.35E−06 |
| | Reproductive System Disease | | |
| 174 | Cell Morphology | abnormal morphology of | 9.59E−06 |
| | | plasma membrane | |
| 175 | Cancer, Organismal Injury and Abnormalities, Reproductive System Disease | carcinoma in breast | 9.87E−06 |
| 176 | Cardiovascular Disease, Organismal Injury and Abnormalities | carotid artery stenosis | 9.88E−06 |
| 177 | Connective Tissue Disorders, Inflammatory Disease, Skeletal and Muscular | Rheumatic Disease | 9.91E−06 |
| | Disorders | | |
| 178 | Cardiovascular Disease | microangiopathy | 9.96E−06 |
| 179 | Cardiovascular Disease, Organismal Injury and Abnormalities | stenosis of aortic valve | 1.06E−05 |
| 180 | Organismal Injury and Abnormalities, Reproductive System Disease | prostatic disease | 1.07E−05 |
| 181 | Cardiovascular Disease, Organismal Injury and Abnormalities | valvulopathy | 1.11E−05 |
| 182 | Cancer, Hematological Disease, Immunological Disease, Organismal Injury and | lymphocytic leukemia | 1.14E−05 |
| | Abnormalities | | |
| 183 | Cell-To-Cell Signaling and Interaction, Nervous System Development and | neurotransmission | 1.18E−05 |
| | Function | | |
| 184 | Cancer, Hematological Disease, Immunological Disease, Organismal Injury and | hematologic cancer | 1.33E−05 |
| | Abnormalities | | |
| 185 | Tissue Morphology | quantity of cells | 1.37E−05 |
| 186 | Neurological Disease, Organismal Injury and Abnormalities | astrocytosis | 1.41E−05 |
| 187 | Cell Morphology | morphology of cells | 1.46E−05 |
| 188 | Cancer, Organismal Injury and Abnormalities, Reproductive System Disease | male genital neoplasm | 1.56E−05 |
| 189 | Cancer, Organismal Injury and Abnormalities, Reproductive System Disease | Prostate Cancer and | 1.58E−05 |
| | | Tumors | |
| 190 | Cell Morphology, Cellular Assembly and Organization, Cellular Development, | formation of plasma | 1.68E−05 |
| | Cellular Function and Maintenance, Cellular Growth and Proliferation | membrane projections | |
| 191 | Organismal Injury and Abnormalities, Renal and Urological Disease | end-stage chronic kidney | 1.68E−05 |
| | | disease | |
| 192 | Organismal Injury and Abnormalities, Renal and Urological Disease | end-stage renal failure | 1.68E−05 |
| 193 | Cancer, Endocrine System Disorders, Organismal Injury and Abnormalities, | endometrioid ovarian | 1.72E−05 |
| | Reproductive System Disease | cancer | |
| 194 | Cancer, Organismal Injury and Abnormalities, Respiratory Disease | non small cell lung | 1.73E−05 |
| | | adenocarcinoma | |
| 195 | Cancer, Organismal Injury and Abnormalities, Reproductive System Disease | prostate cancer | 1.75E−05 |
| 196 | Organismal Injury and Abnormalities | Cytosis | 1.80E−05 |
| 197 | Cardiovascular Disease | atherosclerosis | 1.82E−05 |
| 198 | Organismal Injury and Abnormalities, Renal and Urological Disease | chronic renal failure | 1.97E−05 |
| 199 | Neurological Disease | seizure disorder | 1.97E−05 |
| 200 | Cancer, Cardiovascular Disease, Organismal Injury and Abnormalities | hemangioblastoma | 2.03E−05 |
| 201 | Hereditary Disorder, Neurological Disease, Organismal Injury and Abnormalities, | schizophreniform | 2.08E−05 |
| | Psychological Disorders | psychosis | |
| 202 | Cancer, Organismal Injury and Abnormalities, Reproductive System Disease | breast cancer | 2.19E−05 |
| 203 | Neurological Disease, Psychological Disorders | autism | 2.38E−05 |
| 204 | Cancer, Endocrine System Disorders, Organismal Injury and Abnormalities, | adenosquamous ovarian | 2.44E−05 |
| | Reproductive System Disease | cancer | |
| 205 | Cardiovascular Disease, Organismal Injury and Abnormalities | coronary artery disease | 2.44E−05 |
| 206 | Cancer, Organismal Injury and Abnormalities, Reproductive System Disease | malignant neoplasm of | 2.71E−05 |
| | | male genital organ | |
| 207 | Cancer, Organismal Injury and Abnormalities, Reproductive System Disease | mammary tumor | 2.86E−05 |
| 208 | Cellular Assembly and Organization | organization of cellular | 2.92E−05 |
| | | membrane | |
| 209 | Psychological Disorders | alcoholism | 3.38E−05 |
| 210 | Neurological Disease, Psychological Disorders | Schizophrenia | 3.43E−05 |
| 211 | Ophthalmic Disease, Organismal Injury and Abnormalities | degeneration of eye | 3.55E−05 |
| 212 | Cancer, Hematological Disease, Immunological Disease, Organismal Injury and | myeloid leukemia | 3.60E−05 |
| | Abnormalities | | |
| 213 | Tissue Development | development of epithelial | 3.90E−05 |
| | | tissue | |
| 214 | Cardiovascular Disease, Organismal Injury and Abnormalities | valvular regurgitation of | 4.17E−05 |
| | | atrioventricular valve | |
| 215 | Cancer, Organismal Injury and Abnormalities | metastasis | 4.46E−05 |
| 216 | Organismal Injury and Abnormalities, Renal and Urological Disease | end stage renal disease | 4.46E−05 |
| 217 | Psychological Disorders | Substance-Related | 4.60E−05 |
| | | Disorders | |
| 218 | Psychological Disorders | drug dependence | 4.73E−05 |
| 219 | Cell Morphology, Nervous System Development and Function | innervation | 5.01E−05 |
| 220 | Neurological Disease | hydrocephalus | 5.05E−05 |
| 221 | Cellular Development, Cellular Growth and Proliferation | cell proliferation of tumor | 5.06E−05 |
| | | cell lines | |
| 222 | Cell Death and Survival | killing of leukemia cell | 5.10E−05 |
| | | lines | |
| 223 | Infectious Diseases | infection by Retroviridae | 5.21E−05 |
| 224 | Hereditary Disorder, Neurological Disease, Organismal Injury and Abnormalities, | schizoaffective disorder | 5.23E−05 |
| | Psychological Disorders | | |
| 225 | Cancer, Neurological Disease, Organismal Injury and Abnormalities | glioblastoma cancer | 5.27E−05 |
| 226 | Hereditary Disorder, Organismal Injury and Abnormalities | autosomal dominant | 5.52E−05 |
| | | disease | |
| 227 | Cancer, Hematological Disease, Immunological Disease, Organismal Injury and | acute myeloid leukemia | 5.58E−05 |
| | Abnormalities | with PKC412 resistance | |
| 228 | Gastrointestinal Disease, Inflammatory Disease | Crohn's disease | 5.62E−05 |
| 229 | Behavior | social behavior | 5.63E−05 |
| 230 | Energy Production, Lipid Metabolism, Small Molecule Biochemistry | oxidation of lipid | 5.77E−05 |
| 231 | Hereditary Disorder, Neurological Disease, Organismal Injury and Abnormalities | idiopathic generalized | 5.96E−05 |
| | | epilepsy | |
| 232 | Cancer, Organismal Injury and Abnormalities, Respiratory Disease | tumor antigen-positive | 5.96E−05 |
| | | non-small cell lung cancer | |
| 233 | Gastrointestinal Disease, Inflammatory Disease | Inflammatory Bowel | 5.98E−05 |
| | | Disease | |
| 234 | Developmental Disorder, Organismal Injury and Abnormalities | congenital malformation | 6.00E−05 |
| | | of genitourinary system | |
| 235 | Cancer, Organismal Injury and Abnormalities | growth of tumor | 6.05E−05 |
| 236 | Cardiovascular Disease, Neurological Disease | cerebrovascular | 6.12E−05 |
| | | dysfunction | |
| 237 | Neurological Disease | neurological signs | 6.37E−05 |
| 238 | Cardiovascular Disease | carotid artery disease | 6.50E−05 |
| 239 | Cell Morphology, Cellular Assembly and Organization, Cellular Development, | neuritogenesis | 6.61E−05 |
| | Cellular Function and Maintenance, Cellular Growth and Proliferation, Nervous | | |
| | System Development and Function, Tissue Development | | |
| 240 | Neurological Disease | degeneration of nervous | 6.65E−05 |
| | | system | |
| 241 | Cell Morphology, Nervous System Development and Function, Tissue Morphology | abnormal morphology of | 6.66E−05 |
| | | neurons | |
| 242 | Behavior | hyperactivity | 6.81E−05 |
| 243 | Developmental Disorder, Organismal Injury and Abnormalities, Reproductive | congenital anomaly of | 7.38E−05 |
| | System Disease | genital system | |
| 244 | Cellular Compromise, Neurological Disease, Organismal Injury and Abnormalities | neurodegeneration of | 7.75E−05 |
| | | brain cells | |
| 245 | Cell Death and Survival, Cellular Compromise, Neurological Disease, Organismal | neurodegeneration of | 7.76E−05 |
| | Injury and Abnormalities, Tissue Morphology | Purkinje cells | |
| 246 | Cancer, Organismal Injury and Abnormalities | advanced malignant | 7.91E−05 |
| | | tumor | |
| 247 | Cancer, Hematological Disease, Immunological Disease, Organismal Injury and | acute lymphocytic | 8.08E−05 |
| | Abnormalities | leukemia | |
| 248 | Cell-To-Cell Signaling and Interaction, Nervous System Development and | synaptic transmission of | 8.79E−05 |
| | Function | cerebral cortex cells | |
| 249 | Ophthalmic Disease, Organismal Injury and Abnormalities | retinal degeneration | 8.92E−05 |
| 250 | Cancer, Gastrointestinal Disease, Hepatic System Disease, Organismal Injury and | malignant gallbladder | 9.24E−05 |
| | Abnormalities | neoplasia | |
| 251 | Endocrine System Disorders, Gastrointestinal Disease, Immunological Disease, | insulin-dependent | 9.71E−05 |
| | Metabolic Disease | diabetes mellitus | |
| 252 | Cellular Movement | cell movement | 9.74E−05 |
| 253 | Respiratory Disease | chronic obstructive | 9.75E−05 |
| | | pulmonary disease | |
| 254 | Cell-To-Cell Signaling and Interaction, Nervous System Development and | synaptic transmission | 9.77E−05 |
| | Function | | |
| 255 | Cell Morphology, Nervous System Development and Function, Tissue Morphology | morphology of neurons | 1.02E−04 |
| 256 | Infectious Diseases | infection by lentivirus | 1.05E−04 |
| 257 | Neurological Disease | generalized epilepsy | 1.06E−04 |
| 258 | Neurological Disease | dyskinesia | 1.18E−04 |
| 259 | Infectious Diseases | infection by RNA virus | 1.24E−04 |
| 260 | Molecular Transport | secretion of molecule | 1.25E−04 |
| 261 | Cancer, Gastrointestinal Disease, Hepatic System Disease, Organismal Injury and | biliary tract carcinoma | 1.26E−04 |
| | Abnormalities | | |
| 262 | Psychological Disorders | drug abuse | 1.26E−04 |
| 263 | Cardiovascular System Development and Function | development of | 1.28E−04 |
| | | vasculature | |
| 264 | Skeletal and Muscular System Development and Function | morphology of muscle | 1.32E−04 |
| 265 | Behavior | hypnosis | 1.36E−04 |
| 266 | Cellular Function and Maintenance | regulation of osteoclasts | 1.36E−04 |
| 267 | Organismal Survival | perinatal death | 1.36E−04 |
| 268 | Nervous System Development and Function, Organ Morphology, Organismal | abnormal morphology of | 1.40E−04 |
| | Development | cerebellum | |
| 269 | Neurological Disease | amyotrophic lateral | 1.41E−04 |
| | | sclerosis | |
| 270 | Cancer, Organismal Injury and Abnormalities | benign neoplasia | 1.50E−04 |
| 271 | Neurological Disease, Psychological Disorders | insomnia | 1.53E−04 |
| 272 | Cardiovascular System Development and Function | morphology of | 1.56E−04 |
| | | cardiovascular system | |
| 273 | Psychological Disorders | generalized anxiety | 1.58E−04 |
| | | disorder | |
| 274 | Cell-To-Cell Signaling and Interaction, Nervous System Development and | action potential of cells | 1.61E−04 |
| | Function | | |
| 275 | Skeletal and Muscular System Development and Function | quantity of bone | 1.61E−04 |
| 276 | Connective Tissue Disorders, Developmental Disorder, Skeletal and Muscular | pectus excavatum | 1.65E−04 |
| | Disorders | | |
| 277 | Cardiovascular System Development and Function | abnormal morphology of | 1.65E−04 |
| | | cardiovascular system | |
| 278 | Neurological Disease, Organismal Injury and Abnormalities | subarachnoid hemorrhage | 1.67E−04 |
| 279 | Cell-To-Cell Signaling and Interaction, Reproductive System Development and | binding of gonadal cell | 1.76E−04 |
| | Function | lines | |
| 280 | Behavior | locomotion | 1.83E−04 |
| 281 | Embryonic Development, Lymphoid Tissue Structure and Development, Organ | formation of lymph node | 1.92E−04 |
| | Development, Organismal Development, Tissue Development | | |
| 282 | Neurological Disease, Organismal Injury and Abnormalities | damage of brain | 1.95E−04 |
| 283 | Auditory Disease, Inflammatory Disease | otitis media | 1.96E−04 |
| 284 | Embryonic Development, Organismal Development | development of abdomen | 1.98E−04 |
| 285 | Organismal Functions | grasping | 1.98E−04 |
| 286 | Cell Signaling, Small Molecule Biochemistry, Vitamin and Mineral Metabolism | oscillation of Ca2+ | 1.98E−04 |
| 287 | Cell Death and Survival | neuronal cell death | 1.99E−04 |
| 288 | Lipid Metabolism, Small Molecule Biochemistry | synthesis of lipid | 2.03E−04 |
| 289 | Cell-To-Cell Signaling and Interaction, Humoral Immune Response, Inflammatory | immune response of B | 2.07E−04 |
| | Response | lymphocytes | |
| 290 | Neurological Disease, Respiratory Disease, Skeletal and Muscular Disorders | laryngismus | 2.07E−04 |
| 291 | Cancer, Endocrine System Disorders, Gastrointestinal Disease, Organismal Injury | pancreatic intraepithelial | 2.11E−04 |
| | and Abnormalities | neoplasia | |
| 292 | Embryonic Development, Organ Development, Organ Morphology, Organismal | abnormal morphology of | 2.15E−04 |
| | Development, Tissue Development, Tissue Morphology, Visual System | bruch membrane tissue | |
| | Development and Function | | |
| 293 | Organismal Injury and Abnormalities | low back pain | 2.18E−04 |
| 294 | Lipid Metabolism, Small Molecule Biochemistry | removal of lipid | 2.18E−04 |
| 295 | Infectious Diseases | HIV infection | 2.20E−04 |
| 296 | Gastrointestinal Disease | nausea | 2.33E−04 |
| 297 | Cellular Compromise | degeneration of cells | 2.34E−04 |
| 298 | Cell-To-Cell Signaling and Interaction, Nervous System Development and | long-term potentiation | 2.35E−04 |
| | Function | | |
| 299 | Nervous System Development and Function | abnormal morphology of | 2.36E−04 |
| | | cranial nerve | |
| 300 | Cancer, Gastrointestinal Disease, Hepatic System Disease, Organismal Injury and | gall bladder | 2.36E−04 |
| | Abnormalities | adenocarcinoma | |
| 301 | Developmental Disorder, Organismal Injury and Abnormalities, Reproductive | congenital malformation | 2.37E−04 |
| | System Disease | of genital organs | |
| 302 | Lipid Metabolism, Small Molecule Biochemistry | conversion of fatty acid | 2.41E−04 |
| 303 | Neurological Disease, Psychological Disorders | Sleep Disorders | 2.51E−04 |
| 304 | Organismal Injury and Abnormalities | Pathological Cyst | 2.52E−04 |
| 305 | Connective Tissue Disorders, Developmental Disorder, Organismal Development, | abnormal morphology of | 2.56E−04 |
| | Skeletal and Muscular Disorders | lip | |
| 306 | Cellular Function and Maintenance, Hematological System Development and | engulfment of pyrenocytes | 2.56E−04 |
| | Function, Hematopoiesis | | |
| 307 | Cardiovascular Disease, Infectious Diseases, Organismal Injury and | infection of heart | 2.56E−04 |
| | Abnormalities | | |
| 308 | Lipid Metabolism, Small Molecule Biochemistry | metabolism of | 2.56E−04 |
| | | galactosylceramide | |
| 309 | Lipid Metabolism, Small Molecule Biochemistry | metabolism of | 2.56E−04 |
| | | glucosylceramide | |
| 310 | Cell Cycle, Cellular Development | oncogene-induced | 2.56E−04 |
| | | senescence of melanocytes | |
| 311 | Behavior | emotional behavior | 2.77E−04 |
| 312 | Psychological Disorders | Psychosis | 2.88E−04 |
| 313 | Gastrointestinal Disease | chemotherapy sickness | 2.92E−04 |
| 314 | Organismal Injury and Abnormalities, Renal and Urological Disease | renal cyst | 2.94E−04 |
| 315 | Cancer, Gastrointestinal Disease, Organismal Injury and Abnormalities | cecum adenocarcinoma | 2.95E−04 |
| 316 | Neurological Disease | seizures | 3.07E−04 |
| 317 | Nervous System Development and Function, Organ Morphology, Organismal | abnormal morphology of | 3.08E−04 |
| | Development | telencephalon | |
| 318 | Connective Tissue Disorders, Dental Disease, Developmental Disorder, | dental caries | 3.11E−04 |
| | Gastrointestinal Disease, Skeletal and Muscular Disorders | | |
| 319 | Cellular Development, Cellular Growth and Proliferation, Hematological System | proliferation of TREG | 3.11E−04 |
| | Development and Function | cells | |
| 320 | Cardiovascular Disease, Organismal Injury and Abnormalities | valvular regurgitation of | 3.11E−04 |
| | | mitral valve | |
| 321 | Neurological Disease | absence seizure | 3.32E−04 |
| 322 | Hereditary Disorder, Organismal Injury and Abnormalities, Psychological | trichotillomania | 3.32E−04 |
| | Disorders | | |
| 323 | Connective Tissue Development and Function, Skeletal and Muscular System | quantity of bone cells | 3.36E−04 |
| | Development and Function, Tissue Morphology | | |
| 324 | Neurological Disease | progressive motor | 3.37E−04 |
| | | neuropathy | |
| 325 | Neurological Disease, Skeletal and Muscular Disorders | neuromuscular disease | 3.40E−04 |
| 326 | Cellular Development, Cellular Growth and Proliferation | cell proliferation of breast | 3.44E−04 |
| | | cancer cell lines | |
| 327 | Humoral Immune Response, Lymphoid Tissue Structure and Development | formation of germinal | 3.56E−04 |
| | | center | |
| 328 | Connective Tissue Development and Function, Tissue Morphology | quantity of connective | 3.57E−04 |
| | | tissue cells | |
| 329 | Cellular Movement | migration of cells | 3.70E−04 |
| 330 | Auditory Disease, Inflammatory Disease | chronic otitis media | 3.76E−04 |
| 331 | Cancer, Organismal Injury and Abnormalities, Respiratory Disease | stage 4 EGFR expressing | 3.76E−04 |
| | | non-small cell lung cancer | |
| 332 | Behavior | motor learning | 3.76E−04 |
| 333 | Neurological Disease, Psychological Disorders | disorder of basal ganglia | 3.82E−04 |
| 334 | Nervous System Development and Function, Organ Morphology, Organismal | morphology of cerebral | 4.16E−04 |
| | Development | cortex | |
| 335 | Cell Death and Survival | cellular degradation | 4.17E−04 |
| 336 | Inflammatory Response | graft rejection | 4.24E−04 |
| 337 | Cardiovascular System Development and Function, Organ Morphology, | morphology of trabeculae | 4.24E−04 |
| | Organismal Development | carne | |
| 338 | Psychological Disorders | panic disorder | 4.24E−04 |
| 339 | Cellular Development, Cellular Growth and Proliferation, Embryonic | formation of brain cells | 4.27E−04 |
| | Development, Nervous System Development and Function, Organ Development, | | |
| | Organismal Development, Tissue Development | | |
| 340 | Cancer, Gastrointestinal Disease, Organismal Injury and Abnormalities | small intestine cancer | 4.27E−04 |
| 341 | Embryonic Development, Nervous System Development and Function, Organ | formation of | 4.30E−04 |
| | Development, Organismal Development, Tissue Development | telencephalon | |
| 342 | Energy Production, Lipid Metabolism, Small Molecule Biochemistry | oxidation of fatty acid | 4.49E−04 |
| 343 | Auditory Disease, Neurological Disease | noise-induced hearing loss | 4.49E−04 |
| 344 | Cancer, Organismal Injury and Abnormalities, Respiratory Disease | stage 3B EGFR mutation | 4.49E−04 |
| | | positive non-small cell | |
| | | lung cancer | |
| 345 | Cardiovascular Disease, Neurological Disease | disruption of blood-brain | 4.49E−04 |
| | | barrier | |
| 346 | Developmental Disorder, Hereditary Disorder, Metabolic Disease, Organismal | lipofuscinosis | 4.49E−04 |
| | Injury and Abnormalities | | |
| 347 | Neurological Disease, Psychological Disorders | primary insomnia | 4.49E−04 |
| 348 | Cancer, Organismal Injury and Abnormalities | stage 3A cancer | 4.49E−04 |
| 349 | Neurological Disease | early-onset neurological | 4.61E−04 |
| | | disorder | |
| 350 | Lipid Metabolism, Molecular Transport, Small Molecule Biochemistry | secretion of lipid | 4.74E−04 |
| 351 | Psychological Disorders | Anxiety Disorders | 5.03E−04 |
| 352 | Behavior | anxiety-like behavior | 5.08E−04 |
| 353 | Embryonic Development, Organ Development, Organ Morphology, Organismal | abnormal morphology of | 5.12E−04 |
| | Development, Respiratory System Development and Function, Tissue | lung | |
| | Development | | |
| 354 | Cell Signaling, Cellular Function and Maintenance, Small Molecule Biochemistry, | homeostasis of Ca2+ | 5.16E−04 |
| | Vitamin and Mineral Metabolism | | |
| 355 | Cell Death and Survival | cell death of progenitor | 5.32E−04 |
| | | cells | |
| 356 | Neurological Disease | myoclonic seizure | 5.32E−04 |
| 357 | Developmental Disorder, Hereditary Disorder, Neurological Disease, Organismal | nonsyndromic familial | 5.32E−04 |
| | Injury and Abnormalities | mental retardation | |
| 358 | Skeletal and Muscular System Development and Function | abnormal morphology of | 5.36E−04 |
| | | muscle | |
| 359 | Organismal Development | abnormal morphology of | 5.46E−04 |
| | | abdomen | |
| 360 | Neurological Disease | late-onset neurological | 5.53E−04 |
| | | disorder | |
| 361 | Cellular Movement, Nervous System Development and Function | guidance of axons | 5.64E−04 |
| 362 | Organismal Injury and Abnormalities | stenosis | 5.64E−04 |
| 363 | Organismal Injury and Abnormalities, Renal and Urological Disease | chronic kidney disease | 5.99E−04 |
| 364 | Psychological Disorders | impulse control disorder | 6.02E−04 |
| 365 | Embryonic Development, Nervous System Development and Function, Organ | abnormal morphology of | 6.08E−04 |
| | Development, Organ Morphology, Organismal Development, Tissue Development, | neurosensory retina | |
| | Visual System Development and Function | | |
| 366 | Neurological Disease, Psychological Disorders | daytime sleepiness | 6.10E−04 |
| 367 | Lipid Metabolism, Molecular Transport, Small Molecule Biochemistry | release of oleic acid | 6.10E−04 |
| 368 | Neurological Disease | Neurodegeneration | 6.18E−04 |
| 369 | Cardiovascular System Development and Function, Organ Morphology, | morphology of heart | 6.21E−04 |
| | Organismal Development | | |
| 370 | Behavior | anxiety | 6.26E−04 |
| 371 | Cellular Function and Maintenance | turnover of cells | 6.26E−04 |
| 372 | Metabolic Disease, Neurological Disease, Psychological Disorders | Alzheimer's disease | 6.28E−04 |
| 373 | Neurological Disease, Psychological Disorders | Dementia | 6.29E−04 |
| 374 | Cell Death and Survival, Cellular Compromise, Neurological Disease, Tissue | degeneration of neurons | 6.38E−04 |
| | Morphology | | |
| 375 | Cancer, Organismal Injury and Abnormalities, Renal and Urological Disease | renal cell cancer | 6.45E−04 |
| 376 | Organismal Injury and Abnormalities | postoperative pain | 6.49E−04 |
| 377 | Gastrointestinal Disease | vomiting | 6.49E−04 |
| 378 | Neurological Disease | damage of nervous system | 6.58E−04 |
| 379 | Lipid Metabolism, Molecular Transport, Small Molecule Biochemistry | clearance of lipid | 6.65E−04 |
| 380 | Cell-To-Cell Signaling and Interaction, Nervous System Development and | synaptic transmission of | 6.65E−04 |
| | Function | hippocampal cells | |
| 381 | Behavior | spatial learning | 6.76E−04 |
| 382 | Nervous System Development and Function, Organ Morphology, Organismal | abnormal morphology of | 7.03E−04 |
| | Development | cerebral cortex | |
| 383 | Neurological Disease, Psychological Disorders | attention deficit | 7.08E−04 |
| | | hyperactivity disorder | |
| 384 | Hereditary Disorder, Ophthalmic Disease, Organismal Injury and Abnormalities | Hereditary Eye Disease | 7.20E−04 |
| 385 | Cellular Development, Cellular Growth and Proliferation, Embryonic | development of cerebral | 7.30E−04 |
| | Development, Nervous System Development and Function, Organ Development, | cortex cells | |
| | Organismal Development, Tissue Development | | |
| 386 | Cell Morphology, Cellular Assembly and Organization, Cellular Function and | formation of cilia | 7.30E−04 |
| | Maintenance | | |
| 387 | Developmental Disorder, Endocrine System Disorders, Organismal Injury and | cryptorchidism | 7.38E−04 |
| | Abnormalities, Reproductive System Disease | | |
| 388 | Cellular Movement, Hair and Skin Development and Function | migration of epithelial cell | 7.40E−04 |
| | | lines | |
| 389 | Nervous System Development and Function, Organ Morphology, Organismal | abnormal morphology of | 7.58E−04 |
| | Development | visual cortex | |
| 390 | Cell Death and Survival, Cellular Function and Maintenance | clearance of Salmonella | 7.58E−04 |
| | | enterica subsp. enterica | |
| | | serovar Dublin strain | |
| | | SL5631 | |
| 391 | Cancer, Cellular Development, Cellular Growth and Proliferation, Organismal | colony formation of | 7.58E−04 |
| | Injury and Abnormalities, Tumor Morphology | leukemia stem cells | |
| 392 | Cell Cycle, Cell-To-Cell Signaling and Interaction, Hematological System | effector phase of naive T | 7.58E−04 |
| | Development and Function, Inflammatory Response | lymphocytes | |
| 393 | Cell Cycle, Respiratory System Development and Function | entry into S phase of lung | 7.58E−04 |
| | | cell lines | |
| 394 | Cancer, Gastrointestinal Disease, Organismal Injury and Abnormalities | gastrointestinal stromal | 7.58E−04 |
| | | tumor in gastrointestinal | |
| | | tract | |
| 395 | Cancer, Cellular Movement, Organismal Injury and Abnormalities | invasion of hepatocytes | 7.58E−04 |
| 396 | Neurological Disease | misrouting of axons | 7.58E−04 |
| 397 | Neurological Disease, Organismal Injury and Abnormalities | neurodegeneration of | 7.58E−04 |
| | | spinal cord | |
| 398 | Energy Production, Lipid Metabolism, Small Molecule Biochemistry | oxidation of lauric acid | 7.58E−04 |
| 399 | Connective Tissue Disorders | pseudoxanthoma | 7.58E−04 |
| | | elasticum | |
| 400 | Cellular Function and Maintenance | regulation of chondrocytes | 7.58E−04 |
| 401 | Cardiovascular System Development and Function, Organ Morphology, | abnormal morphology of | 7.59E−04 |
| | Organismal Development | heart | |
| 402 | Nervous System Development and Function | morphology of nerves | 7.87E−04 |
| 403 | Organismal Injury and Abnormalities | formation of pathological | 7.88E−04 |
| | | cyst | |
| 404 | Cell Signaling | enzyme linked receptor | 7.88E−04 |
| | | protein signaling pathway | |
| 405 | Nervous System Development and Function, Organ Morphology, Organismal | abnormal morphology of | 8.01E−04 |
| | Development | forebrain | |
| 406 | Cardiovascular System Development and Function, Cell-To-Cell Signaling and | action potential of | 8.04E−04 |
| | Interaction, Nervous System Development and Function, Skeletal and Muscular | cardiomyocytes | |
| | System Development and Function | | |
| 407 | Ophthalmic Disease, Organismal Injury and Abnormalities | age-related macular | 8.17E−04 |
| | | degeneration | |
| 408 | Neurological Disease, Organismal Injury and Abnormalities | brain atrophy | 8.28E−04 |
| 409 | Cellular Development, Cellular Growth and Proliferation | proliferation of | 8.28E−04 |
| | | lymphoblastoid cell lines | |
| 410 | Organismal Injury and Abnormalities | Bleeding | 8.29E−04 |
| 411 | Cardiovascular System Development and Function, Organ Morphology, | morphology of cardiac | 8.30E−04 |
| | Organismal Development, Skeletal and Muscular System Development and | muscle | |
| | Function | | |
| 412 | Neurological Disease, Psychological Disorders, Skeletal and Muscular Disorders | Parkinson's disease | 8.87E−04 |
| 413 | Cancer, Organismal Injury and Abnormalities, Reproductive System Disease | prostatic carcinoma | 9.00E−04 |
| 414 | Cardiovascular System Development and Function, Organ Morphology, | morphology of heart | 9.15E−04 |
| | Organismal Development | ventricle | |
| 415 | Neurological Disease | epileptic encephalopathy | 9.50E−04 |
| 416 | Tissue Morphology | abnormal morphology of | 9.58E−04 |
| | | epithelial tissue | |
| 417 | Nervous System Development and Function | abnormal morphology of | 9.75E−04 |
| | | vestibulocochlear nerve | |
| 418 | Neurological Disease | unconsciousness | 9.75E−04 |
| 419 | Lipid Metabolism, Small Molecule Biochemistry | homeostasis of lipid | 9.85E−04 |
| 420 | Cardiovascular System Development and Function | morphology of vessel | 9.91E−04 |
| 421 | Cellular Development, Cellular Growth and Proliferation, Hematological System | proliferation of regulatory | 9.95E−04 |
| | Development and Function | T lymphocytes | |
| 422 | Neurological Disease | chorea | 1.01E−03 |
| 423 | Cellular Compromise, Cellular Function and Maintenance | endoplasmic reticulum | 1.01E−03 |
| | | stress response of cells | |
| 424 | Lipid Metabolism, Small Molecule Biochemistry, Vitamin and Mineral Metabolism | homeostasis of cholesterol | 1.01E−03 |
| 425 | Cell-To-Cell Signaling and Interaction | adhesion of connective | 1.02E−03 |
| | | tissue cells | |
| 426 | Nervous System Development and Function, Organ Morphology, Organismal | abnormal morphology of | 1.03E−03 |
| | Development | cerebellar vermis | |
| 427 | Behavior | hyperlocomotion | 1.03E−03 |
| 428 | Cellular Movement, Immune Cell Trafficking, Inflammatory Response | invasion of macrophages | 1.03E−03 |
| 429 | Auditory Disease, Neurological Disease | late-onset hearing loss | 1.03E−03 |
| 430 | Energy Production, Lipid Metabolism, Small Molecule Biochemistry | oxidation of | 1.03E−03 |
| | | polyunsaturated fatty | |
| | | acids | |
| 431 | Cellular Development, Cellular Growth and Proliferation, Embryonic | tubulogenesis of kidney | 1.03E−03 |
| | Development, Organ Development, Organismal Development, Renal and | cell lines | |
| | Urological System Development and Function, Tissue Development | | |
| 432 | Respiratory System Development and Function | abnormal morphology of | 1.05E−03 |
| | | respiratory system | |
| 433 | Cancer, Organismal Injury and Abnormalities | glandular intraepithelial | 1.05E−03 |
| | | neoplasm | |
| 434 | Neurological Disease, Organismal Injury and Abnormalities, Psychological | traumatic brain injury | 1.08E−03 |
| | Disorders | | |
| 435 | Cancer, Neurological Disease, Organismal Injury and Abnormalities | astrocytoma | 1.10E−03 |
| 436 | Digestive System Development and Function | abnormal morphology of | 1.11E−03 |
| | | digestive system | |
| 437 | Cell-To-Cell Signaling and Interaction, Hematological System Development and | cell-cell adhesion of | 1.12E−03 |
| | Function, Immune Cell Trafficking | leukocytes | |
| 438 | Hereditary Disorder, Neurological Disease, Organismal Injury and Abnormalities | severe myoclonic epilepsy | 1.12E−03 |
| | | in infancy | |
| 439 | Cancer, Neurological Disease, Organismal Injury and Abnormalities | central nervous system | 1.13E−03 |
| | | cancer | |
| 440 | Cell Signaling, Post-Translational Modification | activation of Protein | 1.14E−03 |
| | | kinase | |
| 441 | Cardiovascular Disease, Neurological Disease | stroke | 1.19E−03 |
| 442 | Cell Morphology | sprouting | 1.19E−03 |
| 443 | Organismal Injury and Abnormalities, Skeletal and Muscular Disorders | pain of muscle | 1.20E−03 |
| 444 | Developmental Disorder, Neurological Disease, Psychological Disorders | speech and language | 1.20E−03 |
| | | disorders | |
| 445 | Neurological Disease | acute encephalopathy | 1.20E−03 |
| 446 | Cancer, Organismal Injury and Abnormalities, Renal and Urological Disease | renal cancer | 1.26E−03 |
| 447 | Hematological System Development and Function, Tissue Morphology | quantity of myeloid cells | 1.26E−03 |
| 448 | Cellular Growth and Proliferation | proliferation of cells | 1.27E−03 |
| 449 | Organismal Injury and Abnormalities | fatigue | 1.28E−03 |
| 450 | Cell-To-Cell Signaling and Interaction, Nervous System Development and | GABA-mediated receptor | 1.30E−03 |
| | Function | currents | |
| 451 | Lipid Metabolism, Nucleic Acid Metabolism, Small Molecule Biochemistry | conversion of acyl- | 1.30E−03 |
| | | coenzyme A | |
| 452 | Cell Death and Survival, Cellular Compromise | cytotoxicity of phagocytes | 1.30E−03 |
| 453 | Connective Tissue Disorders, Organismal Injury and Abnormalities, Skeletal and | fracture of bone | 1.31E−03 |
| | Muscular Disorders, Tissue Morphology | | |
| 454 | Psychological Disorders | hyperactive behavior | 1.34E−03 |
| 455 | Cardiovascular System Development and Function, Tissue Development | development of | 1.35E−03 |
| | | cardiovascular tissue | |
| 456 | Organismal Injury and Abnormalities | abnormality of head | 1.37E−03 |
| 457 | Cardiovascular System Development and Function, Embryonic Development, | cardiogenesis | 1.38E−03 |
| | Organ Development, Organismal Development, Tissue Development | | |
| 458 | Hematological Disease, Immunological Disease | chronic | 1.38E−03 |
| | | lymphoproliferative | |
| | | disease | |
| 459 | Cancer, Organismal Injury and Abnormalities, Reproductive System Disease | prostatic adenocarcinoma | 1.40E−03 |
| 460 | Cellular Movement | invasion of brain cancer | 1.43E−03 |
| | | cell lines | |
| 461 | Neurological Disease, Psychological Disorders | bipolar I disorder | 1.43E−03 |
| 462 | Connective Tissue Disorders, Organismal Injury and Abnormalities, Skeletal and | hip fracture | 1.44E−03 |
| | Muscular Disorders, Tissue Morphology | | |
| 463 | Cellular Function and Maintenance | maintenance of leukocytes | 1.44E−03 |
| 464 | Cellular Assembly and Organization | quantity of cellular | 1.44E−03 |
| | | inclusion bodies | |
| 465 | Skeletal and Muscular Disorders | skelatal muscle spasticity | 1.44E−03 |
| 466 | Cell-To-Cell Signaling and Interaction, Nervous System Development and | synaptic transmission of | 1.45E−03 |
| | Function | nervous tissue | |
| 467 | Cancer, Neurological Disease, Organismal Injury and Abnormalities | gliomatosis | 1.49E−03 |
| 468 | Cancer, Organismal Injury and Abnormalities, Respiratory Disease | EGFR exon 19 deletion | 1.50E−03 |
| | | positive EGFR L858R | |
| | | mutation positive mixed | |
| | | cell non-small cell lung | |
| | | carcinoma | |
| 469 | Cancer, Organismal Injury and Abnormalities, Respiratory Disease | EGFR(L858R) positive | 1.50E−03 |
| | | metastatic non-small cell | |
| | | lung cancer | |
| 470 | Cancer, Organismal Injury and Abnormalities, Respiratory Disease | HER2 mutation positive | 1.50E−03 |
| | | non-small cell lung cancer | |
| 471 | Auditory Disease, Cellular Compromise, Organismal Injury and Abnormalities, | degeneration of pillar cells | 1.50E−03 |
| | Tissue Morphology | | |
| 472 | Cell-To-Cell Signaling and Interaction, Nervous System Development and | depotentiation of synapse | 1.50E−03 |
| | Function | | |
| 473 | Neurological Disease, Psychological Disorders | drowsiness | 1.50E−03 |
| 474 | Drug Metabolism, Molecular Transport | efflux of mitoxantrone | 1.50E−03 |
| 475 | Cellular Movement | invasion of pneumocytes | 1.50E−03 |
| 476 | Cancer, Organismal Injury and Abnormalities | locally advanced head and | 1.50E−03 |
| | | neck squamous cell | |
| | | carcinoma | |
| 477 | Carbohydrate Metabolism, Lipid Metabolism, Small Molecule Biochemistry | metabolism of | 1.50E−03 |
| | | erythromycin | |
| 478 | Drug Metabolism, Endocrine System Development and Function, Lipid | metabolism of | 1.50E−03 |
| | Metabolism, Small Molecule Biochemistry, Vitamin and Mineral Metabolism | testosterone | |
| 479 | Cancer, Organismal Injury and Abnormalities, Respiratory Disease | metastatic EGFR (L858R) | 1.50E−03 |
| | | positive lung | |
| | | adenocarcinoma | |
| 480 | Cancer, Organismal Injury and Abnormalities | metastatic EGFR G719X | 1.50E−03 |
| | | mutation positive large | |
| | | cell carcinoma | |
| 481 | Cancer, Organismal Injury and Abnormalities, Respiratory Disease | metastatic EGFR G719X | 1.50E−03 |
| | | mutation positive lung | |
| | | adenocarcinoma | |
| 482 | Cancer, Organismal Injury and Abnormalities, Respiratory Disease | metastatic EGFR G719X | 1.50E−03 |
| | | mutation positive non- | |
| | | small cell lung cancer | |
| 483 | Cancer, Organismal Injury and Abnormalities, Respiratory Disease | metastatic EGFR G719X | 1.50E−03 |
| | | mutation positive | |
| | | squamous cell lung | |
| | | carcinoma | |
| 484 | Cancer, Organismal Injury and Abnormalities | metastatic EGFR L858R | 1.50E−03 |
| | | mutation positive large | |
| | | cell carcinoma | |
| 485 | Cancer, Organismal Injury and Abnormalities, Respiratory Disease | metastatic EGFR L858R | 1.50E−03 |
| | | mutation positive | |
| | | squamous cell lung | |
| | | carcinoma | |
| 486 | Cancer, Organismal Injury and Abnormalities | metastatic EGFR L861X | 1.50E−03 |
| | | mutation positive large | |
| | | cell carcinoma | |
| 487 | Cancer, Organismal Injury and Abnormalities, Respiratory Disease | metastatic EGFR L861X | 1.50E−03 |
| | | mutation positive lung | |
| | | adenocarcinoma | |
| 488 | Cancer, Organismal Injury and Abnormalities, Respiratory Disease | metastatic EGFR L861X | 1.50E−03 |
| | | mutation positive non- | |
| | | small cell lung cancer | |
| 489 | Cancer, Organismal Injury and Abnormalities, Respiratory Disease | metastatic EGFR L861X | 1.50E−03 |
| | | mutation positive | |
| | | squamous cell lung | |
| | | carcinoma | |
| 490 | Cancer, Organismal Injury and Abnormalities | metastatic EGFR S768I | 1.50E−03 |
| | | mutation positive large | |
| | | cell carcinoma | |
| 491 | Cancer, Organismal Injury and Abnormalities, Respiratory Disease | metastatic EGFR S768I | 1.50E−03 |
| | | mutation positive lung | |
| | | adenocarcinoma | |
| 492 | Cancer, Organismal Injury and Abnormalities, Respiratory Disease | metastatic EGFR S768I | 1.50E−03 |
| | | mutation positive non- | |
| | | small cell lung cancer | |
| 493 | Cancer, Organismal Injury and Abnormalities, Respiratory Disease | metastatic EGFR S768I | 1.50E−03 |
| | | mutation positive | |
| | | squamous cell lung | |
| | | carcinoma | |
| 494 | Cancer, Organismal Injury and Abnormalities | metastatic EGFR exon 19 | 1.50E−03 |
| | | deletion positive large cell | |
| | | carcinoma | |
| 495 | Cancer, Organismal Injury and Abnormalities, Respiratory Disease | metastatic EGFR exon 19 | 1.50E−03 |
| | | deletion positive lung | |
| | | adenocarcinoma | |
| 496 | Cancer, Organismal Injury and Abnormalities, Respiratory Disease | metastatic EGFR exon 19 | 1.50E−03 |
| | | deletion positive | |
| | | squamous cell lung | |
| | | carcinoma | |
| 497 | Cancer, Organismal Injury and Abnormalities, Respiratory Disease | metastatic EGFR | 1.50E−03 |
| | | sensitizing mutation | |
| | | positive large cell lung | |
| | | carcinoma | |
| 498 | Cancer, Organismal Injury and Abnormalities, Respiratory Disease | metastatic progressive | 1.50E−03 |
| | | EGFR activating | |
| | | mutation positive non- | |
| | | squamous non-small cell | |
| | | lung carcinoma | |
| 499 | Cancer, Organismal Injury and Abnormalities, Respiratory Disease | metastatic progressive | 1.50E−03 |
| | | EGFR sensitizing | |
| | | mutation positive non- | |
| | | small cell lung cancer | |
| 500 | Cancer, Organismal Injury and Abnormalities, Respiratory Disease | metastatic recurrent | 1.50E−03 |
| | | laryngeal squamous cell | |
| | | carcinoma | |
| 501 | Cancer, Organismal Injury and Abnormalities, Respiratory Disease | progressive EGFR | 1.50E−03 |
| | | (L858R) positive lung | |
| | | adenocarcinoma | |
| 502 | Cancer, Organismal Injury and Abnormalities | progressive EGFR G719X | 1.50E−03 |
| | | mutation positive large | |
| | | cell carcinoma | |

The top list from Table 4 is specified in more details.
^{A}shows in category details the diseases, abnormalities, developmental roles and other function of the human genes that are targeted by the human CRISPRs;
^{B}shows the specific disease and function annotations;
^{C}shows the significance of the IPA analyses;
column ^{D} which shows the genes involved can be found in List3 hereinbelow.
© 2000-2016 QIAGEN. All rights reserved.

Next to that, IPA upstream analyses also established that the gene targets could be effected in expression by a wide variety of chemicals including pharmaceutical compounds (Table 5).

TABLE 5
List of upstream regulators that effect the expression human CRISPR gene targets.

| ©2000-2016 QIAGEN. All rights | | | |
|---|---|---|---|
| reserved. | | | |
| Upstream Regulator^{A} | Molecule Type^{B} | p-value of overlap^{C} | |
| STAT6 | transcription | 2.63E−06 | [symbol] COS IL15 MMP2 NCOA3 PPARGC1A |
| | regulator | | [symbol] VCAM1 |
| topotecan | chemical drug | 3.64E−06 | [symbol] RB1 GRID1 GRID2 GRIK2 GRM5 |
| nandrolone decanoate | chemical drug | 1.27E−05 | |
| testosterone cypionate | chemical drug | 1.27E−05 | |
| cyclosporin A | biologic drug | 2.97E−05 | [symbol] COS IQGAP2 ITPR2 LBR MMP2 NFKB1 |
| | | | [symbol] FSF11 UTRN VCAM1 |
| methyltestosterone | chemical drug | 3.13E−05 | |
| CFTR | ion channel | 3.17E−05 | [symbol] D17B4 NFKB1 OSBPL1A |
| PPARA | ligand-dependent | 5.09E−05 | [symbol] CFH CYBB CYP1A1 HMGCR HSD17B4 |
| | nuclear receptor | | [symbol] SARDH SC5D VCAM1 |
| HNRNPA2B1 | other | 5.20E−05 | [symbol] AST4 NPR3 PLOD2 PPARGC1A UTRN |
| lipopolysaccharide | chemical drug | 5.28E−05 | [symbol] D22 CD28 CD2AP CDH2 CRHR1 CTSB |
| | | | [symbol] DDIT4 EGR2 EMB EPHX2 FEM1C |
| | | | [symbol] L1RAPIT1 ITGA8 ITGAM ITGAX LIPG |
| | | | [symbol] PPARGC1A RNF19A SOD1 TEK TFEC |
| EGR1 | transcription | 5.36E−05 | [symbol] HPGD ITGAM NFKB1 SOD1 TNFSF11 |
| | regulator | | |
| SSPN | other | 6.20E−05 | |
| Pkc(s) | group | 6.75E−05 | [symbol] EGR2 HMGCR HPGD MMP2 NFKB1 |
| ethanol | chemical- | 9.61E−05 | [symbol] 3 CYP3A5 EPHX1 GABRG2 GPC5 IL15 |
| | endogenous | | [symbol] SF11 VCAM1 |
| | mammalian | | |
| AGT | growth factor | 1.04E−04 | [symbol] CYBB CYP19A1 FOXP2 GABRB3 HMGCR |
| | | | [symbol] LN2 TNFSF11 VCAM1 |
| CREB1 | transcription | 1.38E−04 | [symbol] EGR2 ESRRG GRIK2 GRM5 HMGCR |
| | regulator | | [symbol] C1A SQSTM1 THOP1 TNFSF11 XKR4 |
| hydroxypropyl-beta-cyclodextrin | chemical drug | 1.53E−04 | |
| prednisolone | chemical drug | 1.61E−04 | [symbol] FKB1 NMT2 SOD1 SQSTM1 TNFSF11 |
| NFATC1 | transcription | 1.69E−04 | [symbol] 1 UTRN VCAM1 |
| | regulator | | |
| Alpha 1 antitrypsin | group | 2.18E−04 | |
| PXR ligand-PXR-Retinoic acid- | complex | 3.32E−04 | [symbol] 2 |
| RXRα | | | |
| TSPYL5 | other | 3.33E−04 | [symbol] 1 FUT8 |
| alprazolam | chemical drug | 3.56E−04 | |
| baicalein | chemical- | 4.65E−04 | |
| | endogenous non- | | |
| | mammalian | | |
| CLCN5 | ion channel | 4.84E−04 | |
| MMP7 | peptidase | 4.84E−04 | |
| ANXA7 | ion channel | 5.48E−04 | |
| dimethyl sulfoxide | chemical drug | 6.15E−04 | [symbol] SQSTM1 TNFSF11 VCAM1 |
| Il8r | group | 6.38E−04 | |
| tetrathiomolybdate | chemical drug | 6.38E−04 | |
| methoxyacetic acid | chemical toxicant | 6.48E−04 | |
| CXCL8 | cytokine | 6.89E−04 | [symbol] 2 VCAM1 |
| ESR1 | ligand-dependent | 7.26E−04 | [symbol] ENPK CLN3 CTSB CYP19A1 CYP1A1 |
| | nuclear receptor | | [symbol] OSR1 HMGCR HSD17B4 HYAL1 KLF3- |
| | | | [symbol] D2 PPARGC1A ROBO2 RTP4 SQSTM1 |
| mono-(2-ethylhexyl)phthalate | chemical toxicant | 7.77E−04 | [symbol] 1 PPARGC1A SC5D SOD1 SUCLG2 |
| LDL | complex | 7.98E−04 | [symbol] YOU1 ITPR2 MMP2 NFKB1 OSCAR |
| miR-19b-3p (and other miRNAs | mature microrna | 8.21E−04 | |
| w/seed GUGCAAA) | | | |
| dopamine | chemical- | 8.57E−04 | [symbol] 4 NPR3 SLC6A3 |
| | endogenous | | |
| | mammalian | | |
| pirinixic acid | chemical toxicant | 8.71E−04 | [symbol] A2 CDH2 CFH CTSB CYP1A1 CYP3A5 |
| | | | [symbol] SC5D VCAM1 |
| eplerenone | chemical drug | 9.50E−04 | |
| TICAM1 | other | 1.01E−03 | [symbol] FSF11 TSC22D1 VCAM1 |
| MYD88 | other | 1.01E−03 | [symbol] KB1 SLCO3A1 TFEC TNFSF11 TSC22D1 |
| YWHAG | other | 1.03E−03 | |
| cholesterol | chemical- | 1.04E−03 | [symbol] M ITGAX MMP2 SC5D VCAM1 ZFPM2 |
| | endogenous | | |
| | mammalian | | |
| SETDB1 | enzyme | 1.08E−03 | |
| PRKAA2 | kinase | 1.19E−03 | [symbol] 2 DOCK4 PPARGC1A |
| PURA | transcription | 1.28E−03 | |
| | regulator | | |
| CDH3 | other | 1.28E−03 | |
| bitertanol | chemical drug | 1.28E−03 | |
| HDAC10 | transcription | 1.28E−03 | |
| | regulator | | |
| GABRA1 | ion channel | 1.28E−03 | |
| (−)-isoproterenol | chemical drug | 1.28E−03 | |
| methyl cellosolve | chemical reagent | 1.28E−03 | |
| nitrendipine | chemical drug | 1.28E−03 | |
| elcatonin | biologic drug | 1.28E−03 | |
| dexamethasone | chemical drug | 1.30E−03 | [symbol] C12orf42 CPA4 CRHR1 CTSB CYBB |
| | | | [symbol] 2 FOXN3 GABRB1 GLRA2 IL15 ITGAM |
| | | | [symbol] 2 OSBPL1A PLOD2 PPARGC1A |
| | | | [symbol] SMOC2 SOD1 SYK TNFSF11 TSC22D1 |
| CLEC11A | growth factor | 1.30E−03 | |
| IL4 | cytokine | 1.36E−03 | [symbol] HPGD ICOS IL15 ITGAX LIPG MERTK |
| | | | [symbol] C1A PROS1 SQSTM1 SYK TFEC THOP1 |
| Foxp1 | transcription | 1.37E−03 | |
| | regulator | | |
| 15-deoxy-delta-12 14-PGJ 2 | chemical- | 1.49E−03 | [symbol] NFKB1 SOD1 TNFSF11 VCAM1 |
| | endogenous non- | | |
| | mammalian | | |
| rosiglitazone | chemical drug | 1.52E−03 | [symbol] A1 DLD EPHX2 GYS2 HPGD ITGAM |
| | | | [symbol] TR1 |
| ZBED6 | other | 1.53E−03 | |
| Rhox5 | transcription | 1.56E−03 | |
| | regulator | | |
| Creb | group | 1.58E−03 | [symbol] GABRG2 HPGD ITGAM MMP2 PPARGC1A |
| MET | kinase | 1.85E−03 | [symbol] SC22D1 |
| TCF | group | 1.85E−03 | [symbol] P2 TSC22D1 |
| ZC3H14 | other | 1.87E−03 | |
| PLAUR | transmembrane | 1.90E−03 | |
| | receptor | | |
| APOE | transporter | 2.07E−03 | [symbol] ITGAX LIPG MMP2 SOD1 VCAM1 |
| minocycline | chemical drug | 2.07E−03 | |
| ACOT13 | enzyme | 2.12E−03 | |
| UBQLN1 | other | 2.12E−03 | |
| UTRN | transmembrane | 2.12E−03 | |
| | receptor | | |
| miR-125b-5p (and other miRNAs | mature microrna | 2.20E−03 | [symbol] UVRAG |
| w/seed CCCUGAG) | | | |
| STK4 | kinase | 2.22E−03 | |
| IL10RA | transmembrane | 2.26E−03 | [symbol] 5 DDIT4 GSTM2 HMCN1 HPGD |
| | receptor | | |
| decitabine | chemical drug | 2.27E−03 | [symbol] R2 CASP8 CDH13 CDH2 CYP19A1 |
| | | | [symbol] RTP4 SYK |
| genistein | chemical drug | 2.31E−03 | [symbol] 1 CYP1A1 HMGCR HPGD MMP2 PPT1 |
| MAPK8 | kinase | 2.40E−03 | [symbol] CYP1A1 MMP2 PPARGC1A |
| Akt | group | 2.42E−03 | [symbol] FKB1 PPARGC1A SEMA6D UTRN |
| PRKAA1 | kinase | 2.46E−03 | [symbol] K4 PPARGC1A |
| TNF | cytokine | 2.50E−03 | [symbol] DH2 CRHR1 CTSB CXCL13 CYBB |
| | | | [symbol] A2 GABRG2 GSTM2 GTDC1 HIVEP1 |
| | | | [symbol] R2 KRT6B MMP2 NFKB1 PDPN PLOD2 |
| | | | [symbol] K TNFSF11 TPST1 VAV3 VCAM1 |
| FOS | transcription | 2.50E−03 | [symbol] 2 GRIK2 HPGD MMP2 PDPN PTPN12 |
| | regulator | | XYLT1 |
| dihydrotestosterone | chemical- | 2.55E−03 | [symbol] CYP19A1 FOXP2 GOSR1 HMGCR HPGD |
| | endogenous | | [symbol] S1 SLCO3A1 VCAM1 |
| | mammalian | | |
| GNAS | enzyme | 2.57E−03 | |
| TNFRSF1A | transmembrane | 2.74E−03 | [symbol] IMP2 VCAM1 |
| | receptor | | |
| ADCYAP1 | other | 2.74E−03 | [symbol] GAM LMO1 NPR3 TENM2 TSC22D1 |
| OLR1 | transmembrane | 2.82E−03 | |
| | receptor | | |
| metribolone | chemical reagent | 2.85E−03 | [symbol] P2 PDE4D PROS1 TRIM36 TSC22D1 |
| ESRRA | ligand-dependent | 2.89E−03 | [symbol] ARGC1A PPP2R2B |
| | nuclear receptor | | |
| LY294002 | chemical-kinase | 3.01E−03 | [symbol] CUBN CYP19A1 DHRS2 EGR2 ERBB3 |
| | inhibitor | | VCAM1 |
| emodin | chemical drug | 3.09E−03 | |
| GRK5 | kinase | 3.14E−03 | |
| CXCL5 | cytokine | 3.14E−03 | |
| FES | kinase | 3.14E−03 | |
| LRIG1 | other | 3.14E−03 | |
| phenelzine | chemical drug | 3.14E−03 | |
| bergamottin | chemical- | 3.14E−03 | |
| | endogenous non- | | |
| | mammalian | | |
| dieldrin | chemical toxicant | 3.14E−03 | |
| PD98059 | chemical-kinase | 3.26E−03 | [symbol] C5 HPGD HYOU1 ITGAM ITGAX LDHC |
| | inhibitor | | [symbol] VCAM1 |
| methotrexate | chemical drug | 3.28E−03 | [symbol] D17B4 MASP2 SOD1 TNFSF11 VCAM1 |
| HR | transcription | 3.38E−03 | |
| | regulator | | |
| BAPTA-AM | chemical reagent | 3.38E−03 | |
| tosylphenylalanyl chloromethyl | chemical-protease | 3.50E−03 | |
| ketone | inhibitor | | |
| Mapk | group | 3.54E−03 | [symbol] AM1 |
| H89 | chemical-kinase | 3.55E−03 | [symbol] 1A TNFSF11 VCAM1 |
| | inhibitor | | |
| TGFB1 | growth factor | 3.64E−03 | [symbol] 28 CDH2 CHRNA7 CTSB CYBB DDIT4 |
| | | | [symbol] D IL15 ITGAM ITGAX ITPR2 LBR MMP2 |
| | | | [symbol] 1 PLOD2 PPARGC1A PPT1 PTPN20 |
| | | | [symbol] AM1 XYLT1 ZFPM2 |
| GPX1 | enzyme | 3.68E−03 | |
| ARHGAP21 | other | 3.68E−03 | |
| U73122 | chemical reagent | 3.68E−03 | |
| caffeic acid phenethyl ester | chemical toxicant | 3.72E−03 | [symbol] VCAM1 |
| TP73 | transcription | 3.84E−03 | [symbol] INTEP1 ITGAX MMP2 PDPN PRKAG2 |
| | regulator | | |
| ritonavir | chemical drug | 3.85E−03 | [symbol] GCR SC5D VCAM1 |
| berberine | chemical drug | 3.91E−03 | [symbol] 2 VCAM1 |
| CEBPA | transcription | 3.92E−03 | [symbol] 2 EPHA7 EPHX1 HMGCR HPGD ITGAM |
| | regulator | | |
| RAC2 | enzyme | 4.00E−03 | |
| phorbol myristate acetate | chemical drug | 4.02E−03 | [symbol] CL13 CYBB CYP19A1 CYP1A1 DDIT4 |
| | | | [symbol] KLHL4 LIPG MAST4 MMP2 NFKB1 |
| | | | [symbol] AM1 |
| RNF41 | enzyme | 4.36E−03 | |
| PPP1CA | phosphatase | 4.36E−03 | |
| androstenedione | chemical- | 4.36E−03 | |
| | endogenous | | |
| | mammalian | | |
| Calcineurin A | group | 4.56E−03 | |
| CD86 | transmembrane | 5.08E−03 | |
| | receptor | | |
| cholic acid | chemical- | 5.09E−03 | [symbol] GC1A |
| | endogenous | | |
| | mammalian | | |
| IFI16 | transcription | 5.09E−03 | |
| | regulator | | |
| nifedipine | chemical drug | 5.09E−03 | [symbol] 1 |
| MTA3 | other | 5.15E−03 | |
| diosgenin | chemical- | 5.15E−03 | |
| | endogenous non- | | |
| | mammalian | | |
| TLR4 | transmembrane | 5.33E−03 | [symbol] RK2 MERTK NFKB1 SLCO3A1 TNFSF11 |
| | receptor | | |
| U0126 | chemical-kinase | 5.65E−03 | [symbol] 1 DDAH1 DHRS2 HPGD MMP2 PTPN12 |
| | inhibitor | | [symbol] R1 |
| lithium | chemical drug | 5.69E−03 | |
| 2-amino-5-azotoluene | chemical toxicant | 5.75E−03 | |
| ziprasidone | chemical drug | 5.75E−03 | |
| miR-200b-3p (and other miRNAs | mature microrna | 5.79E−03 | |
| w/seed AAUACUG) | | | |
| Cg | complex | 5.95E−03 | [symbol] EPHA7 HMGCR HPGD MMP2 NPR3 |
| PTEN | phosphatase | 5.99E−03 | [symbol] HMGCR LALBA MMP2 NFKB1 |
| bucladesine | chemical toxicant | 6.00E−03 | [symbol] HMGCR MMP2 NFKB1 PDEAD TNFSF11 |
| NR1H4 | ligand-dependent | 6.13E−03 | [symbol] P2 PPARGC1A SQSTM1 |
| | nuclear receptor | | |
| PELP1 | other | 6.34E−03 | |
| ciglitazone | chemical drug | 6.34E−03 | |
| P38 MAPK | group | 6.36E−03 | [symbol] ITGAM MMP2 NFKB1 PPARGC1A |
| TCF7L2 | transcription | 6.44E−03 | [symbol] KLHL4 LDLRAP1 NKAIN2 PPARGC1A |
| | regulator | | |
| NfkB1-RelA | complex | 6.47E−03 | |
| DICER1 | enzyme | 6.64E−03 | [symbol] OS1 PTPRN2 TEK |
| SNCA | enzyme | 6.68E−03 | |
| RORA | ligand-dependent | 7.07E−03 | [symbol] CR HPGD PPARGC1A UGGT2 |
| | nuclear receptor | | |
| platelet activating factor | chemical- | 7.28E−03 | |
| | endogenous | | |
| | mammalian | | |
| aflatoxin B1 | chemical- | 7.33E−03 | |
| | endogenous non- | | |
| | mammalian | | |
| voltage-gated calcium channel | complex | 7.33E−03 | |
| FFAR1 | g-protein coupled | 7.33E−03 | |
| | receptor | | |
| TPM3 | other | 7.33E−03 | |
| SIAH1 | enzyme | 7.33E−03 | |
| beta-estradiol | chemical- | 7.43E−03 | [symbol] 2 CRHR1 CTSB CUBN CXCL13 CYBB |
| | endogenous | | [symbol] 2 ERBB3 ERBB4 FOXN3 HMGCR HPGD |
| | mammalian | | [symbol] NCOA3 NPR3 PTPN12 RAPSN RTP4 |
| | | | [symbol] SF11 TRHDE TSC22D1 VAV3 VCAM1 |
| SCAP | other | 7.78E−03 | |
| PIM1 | kinase | 7.78E−03 | |
| CIDEC | other | 7.98E−03 | |
| WNT3A | cytokine | 8.13E−03 | [symbol] RAPSN TNFSF11 VCAM1 |
| RASSF1 | other | 8.18E−03 | |
| diethylnitrosamine | chemical toxicant | 8.18E−03 | [symbol] 1 |
| calcitriol | chemical drug | 8.24E−03 | [symbol] 1 ERBB3 HPGD HSD17B4 ITGAM ITGAX |
| NUPR1 | transcription | 8.65E−03 | [symbol] ARS2 LBR P3H1 PCTP PRELID2 RILPL2 |
| | regulator | | |
| amphetamine | chemical drug | 9.01E−03 | |
| miR-451a (and other miRNAs | mature microrna | 9.07E−03 | |
| w/seed AACCGUU) | | | |
| GBX2 | transcription | 9.07E−03 | |
| | regulator | | |
| TAPI-1 | chemical-protease | 9.07E−03 | |
| | inhibitor | | |
| E64d | chemical-protease | 9.07E−03 | |
| | inhibitor | | |
| salmonella minnesota R595 | chemical- | 9.30E−03 | [symbol] 1 VCAM1 |
| lipopolysaccharides | endogenous non- | | |
| | mammalian | | |
| CXCR4 | g-protein coupled | 9.43E−03 | |
| | receptor | | |
| IL32 | cytokine | 9.43E−03 | |
| lysophosphatidic acid | chemical-other | 9.45E−03 | |
| REST | transcription | 9.66E−03 | [symbol] KR4 |
| | regulator | | |
| Focal adhesion kinase | group | 9.68E−03 | |
| NPPB | other | 9.68E−03 | |
| proteasome inhibitor PSI | chemical-protease | 9.68E−03 | |
| | inhibitor | | |
| daunorubicin | chemical drug | 9.68E−03 | |
| DDIT3 | transcription | 1.00E−02 | |
| | regulator | | |
| delta-9-tetrahydrocannabinol | chemical drug | 1.00E−02 | |
| mifepristone | chemical drug | 1.01E−02 | [symbol] GR2 HPGD ITGAM KRT6B NCOA3 |
| paclitaxel | chemical drug | 1.03E−02 | [symbol] A5 DDAH1 DHRS2 PTPN12 TSC22D1 |
| ESR2 | ligand-dependent | 1.04E−02 | [symbol] 11 EMB HPGD HSD17B4 KRT6B MMP2 |
| | nuclear receptor | | |
| vorinostat | chemical drug | 1.06E−02 | [symbol] MP2 TEK |
| HSF2 | transcription | 1.06E−02 | |
| | regulator | | |
| Map3k7 | kinase | 1.06E−02 | |
| atorvastatin | chemical drug | 1.07E−02 | [symbol] D1 TNFSF11 VCAM1 |
| TAF4 | transcription | 1.08E−02 | [symbol] |
| | regulator | | |
| phorbol esters | chemical-other | 1.08E−02 | [symbol] 3 |
| DNA-methyltransferase | group | 1.10E−02 | |
| mCluR | group | 1.10E−02 | |
| Adaptor protein 2 | complex | 1.10E−02 | |
| PLA2G2D | enzyme | 1.10E−02 | |
| Collagen Alpha1 | group | 1.10E−02 | |
| NPPC | other | 1.10E−02 | |
| AFP | transporter | 1.10E−02 | |
| ITGA3 | other | 1.10E−02 | |
| meloxicam | chemical drug | 1.10E−02 | |
| puerarin | chemical drug | 1.10E−02 | |
| gentamicin | chemical drug | 1.11E−02 | [symbol] OU1 SQSTM1 SUOX TSC22D1 VCAM1 |
| trichostatin A | chemical drug | 1.13E−02 | [symbol] CYP1A1 CYP3A5 DHRS2 FUT8 HMGCR |
| | | | [symbol] N VCAM1 |
| 8-bromoguanosine 3′ 5′-cyclic | chemical-kinase | 1.16E−02 | |
| monophosphate | inhibitor | | |
| SCARB1 | transporter | 1.16E−02 | |
| 4-methylnitrosoamino-1-(3- | chemical toxicant | 1.16E−02 | |
| pyridinyl)-1-butanone | | | |
| Sn50 peptide | chemical toxicant | 1.20E−02 | |
| LEP | growth factor | 1.20E−02 | [symbol] HMGCR ITGAM MMP2 PPARGC1A SC5D |
| IL27 | cytokine | 1.20E−02 | [symbol] CAM1 |
| SP600125 | chemical-kinase | 1.22E−02 | [symbol] IL15 TNFSF11 VCAM1 |
| | inhibitor | | |
| CEBPD | transcription | 1.24E−02 | [symbol] SF11 |
| | regulator | | |
| Pro-inflammatory Cytokine | group | 1.27E−02 | |
| FOLR1 | transporter | 1.29E−02 | [symbol] 1 |
| betamethasone | chemical drug | 1.30E−02 | |
| ELK4 | transcription | 1.30E−02 | |
| | regulator | | |
| CLCA2 | ion channel | 1.30E−02 | |
| RALA | enzyme | 1.30E−02 | |
| clotrimazole | chemical drug | 1.30E−02 | |
| palmitic acid | chemical- | 1.31E−02 | [symbol] R2B SQSTM1 VCAM1 |
| | endogenous | | |
| | mammalian | | |
| F3 | transmembrane | 1.34E−02 | |
| | receptor | | |
| SP1 | transcription | 1.34E−02 | CYP19A1 HMGCR IL15 ITGAM ITGAX |
| | regulator | | |
| lenalidomide | chemical drug | 1.35E−02 | [symbol] 4 UTRN |
| DMD | other | 1.43E−02 | [symbol] 1R14C UTRN XK |
| cocaine | chemical drug | 1.43E−02 | [symbol] NFKB1 SLC6A3 |
| CD80 | transmembrane | 1.48E−02 | |
| | receptor | | |
| ricinoleic acid | chemical- | 1.48E−02 | |
| | endogenous non- | | |
| | mammalian | | |
| troleandomycin | chemical drug | 1.48E−02 | |
| nicotinamide-beta-riboside | chemical- | 1.48E−02 | |
| | endogenous | | |
| | mammalian | | |
| 3 7 12-trihydroxycoprostane | chemical- | 1.48E−02 | |
| | endogenous | | |
| | mammalian | | |
| beclomethasone dipropionate | chemical drug | 1.48E−02 | |
| mecarzole | chemical drug | 1.48E−02 | |
| etalocib | chemical drug | 1.48E−02 | |
| hexacosanoyl-coenzyme A | chemical- | 1.48E−02 | |
| | endogenous non- | | |
| | mammalian | | |
| Plc beta | group | 1.48E−02 | |
| ceramidase | group | 1.48E−02 | |
| Abl1/2 | group | 1.48E−02 | |
| artemether | chemical drug | 1.48E−02 | |
| diacyl lipopeptide | chemical-other | 1.48E−02 | |
| AMG-9810 | chemical reagent | 1.48E−02 | |
| levocetirizine | chemical drug | 1.48E−02 | |
| polyglactin 910 | chemical reagent | 1.48E−02 | |
| LRP4 | other | 1.48E−02 | |
| CDC42BPB | kinase | 1.48E−02 | |
| SGSM1 | other | 1.48E−02 | |
| KLHL17 | other | 1.48E−02 | |
| ACER1 | enzyme | 1.48E−02 | |
| harman | chemical- | 1.48E−02 | |
| | endogenous non- | | |
| | mammalian | | |
| DNAJC15 | other | 1.48E−02 | |
| SLC39A4 | transporter | 1.48E−02 | |
| S100A14 | other | 1.48E−02 | |
| SGMS | group | 1.48E−02 | |
| AZD1981 | chemical drug | 1.48E−02 | |
| PF-4523655 | biologic drug | 1.48E−02 | |
| GABRD | ion channel | 1.48E−02 | |
| AHRR | other | 1.48E−02 | |
| CXCR5 | g-protein coupled | 1.48E−02 | |
| | receptor | | |
| mir-584 | microrna | 1.48E−02 | |
| mir-219 | microrna | 1.48E−02 | |
| mir-654 | microrna | 1.48E−02 | |
| GW 6471 | chemical reagent | 1.48E−02 | |
| atacicept | biologic drug | 1.48E−02 | |
| SLC30A9 | transporter | 1.48E−02 | |
| ETF1 | translation | 1.48E−02 | |
| | regulator | | |
| NAPB | transporter | 1.48E−02 | |
| FSCN1 | other | 1.48E−02 | |
| RNF19A | enzyme | 1.48E−02 | |
| SPOCK1 | other | 1.48E−02 | |
| DLGAP3 | other | 1.48E−02 | |
| ADAM19 | peptidase | 1.48E−02 | |
| GTx-560 | chemical reagent | 1.48E−02 | |
| ZNF433 | other | 1.48E−02 | |
| diacetylbis(4- | chemical reagent | 1.48E−02 | |
| methylthiosemicarbazonato)copper | | | |
| (II) | | | |
| cAMP-dependent protein kinase | chemical drug | 1.48E−02 | |
| inhibitor | | | |
| CGP 52608 | chemical reagent | 1.48E−02 | |
| SB202474 | chemical-kinase | 1.48E−02 | |
| | inhibitor | | |
| sterigmatocystin | chemical toxicant | 1.48E−02 | |
| benomyl | chemical toxicant | 1.48E−02 | |
| D-lactic acid | chemical- | 1.48E−02 | |
| | endogenous | | |
| | mammalian | | |
| cinacalcet | chemical drug | 1.48E−02 | |
| flurazepam | chemical drug | 1.48E−02 | |
| thiabendazole | chemical drug | 1.48E−02 | |
| fadrozole | chemical drug | 1.48E−02 | |
| naphthalene-1-2-dione | chemical toxicant | 1.48E−02 | |
| trans-7 8-dihydroxy-7 8- | chemical toxicant | 1.48E−02 | |
| dihydrobenzo(a)pyrene | | | |
| anthracene | chemical toxicant | 1.48E−02 | |
| nabumetone | chemical drug | 1.48E−02 | |
| 2-amino-3-methylimidazo(4 5- | chemical toxicant | 1.48E−02 | |
| f)quinoline | | | |
| 4-cresol | chemical toxicant | 1.48E−02 | |
| gemtuzumab ozogamicin | biologic drug | 1.48E−02 | |
| myrcene | chemical- | 1.48E−02 | |
| | endogenous non- | | |
| | mammalian | | |
| cineole | chemical drug | 1.48E−02 | |
| harmaline | chemical drug | 1.48E−02 | |
| erucic acid | chemical- | 1.48E−02 | |
| | endogenous non- | | |
| | mammalian | | |
| estramustine | chemical drug | 1.48E−02 | |
| Fe3+ | chemical- | 1.48E−02 | |
| | endogenous | | |
| | mammalian | | |
| Cr3+ | chemical reagent | 1.48E−02 | |
| entinostat | chemical drug | 1.49E−02 | |
| flutamide | chemical drug | 1.49E−02 | |
| fulvestrant | chemical drug | 1.51E−02 | [symbol] B3 NCOA3 SOD1 SQSTM1 VCAM1 |
| harmine | chemical- | 1.53E−02 | |
| | endogenous non- | | |
| | mammalian | | |
| EN1 | transcription | 1.53E−02 | |
| | regulator | | |
| mir-218 | microrna | 1.53E−02 | |
| BMX | kinase | 1.53E−02 | |
| UGCG | enzyme | 1.53E−02 | |
| APEX1 | enzyme | 1.53E−02 | |
| CYP51A1 | enzyme | 1.53E−02 | |
| PEX5L | ion channel | 1.53E−02 | |
| pentylenetetrazol | chemical drug | 1.53E−02 | |
| farnesyl pyrophosphate | chemical- | 1.53E−02 | |
| | endogenous | | |
| | mammalian | | |
| morphine | chemical drug | 1.53E−02 | [symbol] PP1R14C |
| fluticasone | chemical drug | 1.56E−02 | [symbol] 1A TPST1 TSC22D1 |
| GATA6 | transcription | 1.56E−02 | [symbol] 1 DLD IIYAL1 |
| | regulator | | |
| NEDD9 | other | 1.60E−02 | |
| IL1A | cytokine | 1.62E−02 | [symbol] IP2 NFKB1 TNFSF11 VCAM1 |
| IL6 | cytokine | 1.64E−02 | [symbol] 3A5 EGR2 HPGD ICOS IL15 ITGAM |
| | | | [symbol] 1A SC5D TNFSF11 VCAM1 |
| MAP3K14 | kinase | 1.65E−02 | |
| CD3E | transmembrane | 1.65E−02 | |
| | receptor | | |
| SB203580 | chemical-kinase | 1.69E−02 | [symbol] MMP2 NFKB1 PPARGC1A TNFSF11 |
| | inhibitor | | |
| camptothecin | chemical drug | 1.70E−02 | [symbol] XN3 FUT8 IQGAP2 MERTK NFKB1 |
| | | | [symbol] M1 |
| ibuprofen | chemical drug | 1.72E−02 | |
| propofol | chemical drug | 1.72E−02 | |
| tyrphostin AG 1478 | chemical-kinase | 1.74E−02 | |
| | inhibitor | | |
| HOXA10 | transcription | 1.75E−02 | [symbol] S1 RIT2 |
| | regulator | | |
| 15(S)-HETE | chemical- | 1.76E−02 | |
| | endogenous | | |
| | mammalian | | |
| dactolisib | chemical drug | 1.76E−02 | |
| LTB4R | g-protein coupled | 1.76E−02 | |
| | receptor | | |
| gallic acid | chemical- | 1.76E−02 | |
| | endogenous non- | | |
| | mammalian | | |
| spermidine | chemical- | 1.76E−02 | |
| | endogenous | | |
| | mammalian | | |
| Cd2+ | chemical toxicant | 1.76E−02 | |
| INSIC1 | other | 1.78E−02 | |
| TGFB3 | growth factor | 1.78E−02 | [symbol] C1A |
| isobutylmethylxanthine | chemical toxicant | 1.80E−02 | [symbol] C1A SYK |
| INSR | kinase | 1.82E−02 | [symbol] SD17B4 MMP2 PPARGC1A SC5D |
| CASZ1 | enzyme | 1.84E−02 | |
| MAFB | transcription | 1.84E−02 | |
| | regulator | | |
| propranolol | chemical drug | 1.84E−02 | |
| RAC1 | enzyme | 1.85E−02 | |
| DOCK8 | other | 1.91E−02 | |
| 5-hydroxytryptamine | chemical- | 1.91E−02 | |
| | endogenous | | |
| | mammalian | | |
| LHX1 | transcription | 1.92E−02 | |
| | regulator | | |
| ANGPT1 | growth factor | 1.98E−02 | |
| verapamil | chemical drug | 1.98E−02 | |
| PRKCA | kinase | 2.00E−02 | |
| FOSL1 | transcription | 2.01E−02 | |
| | regulator | | |
| leukotriene C4 | chemical- | 2.02E−02 | |
| | endogenous | | |
| | mammalian | | |
| fluoride | chemical- | 2.02E−02 | |
| | endogenous | | |
| | mammalian | | |
| PD 0325901 | chemical drug | 2.02E−02 | |
| Fcgr3 | group | 2.02E−02 | |
| Sod | group | 2.02E−02 | |
| ADRBK1 | kinase | 2.02E−02 | |
| DDR1 | kinase | 2.02E−02 | |
| TNFAIP2 | other | 2.02E−02 | |
| EDNRA | transmembrane | 2.02E−02 | |
| | receptor | | |
| ENPP2 | enzyme | 2.02E−02 | |
| isoquercitrin | chemical drug | 2.02E−02 | |
| kainic acid | chemical toxicant | 2.04E−02 | [symbol] O1 |
| SASH1 | other | 2.10E−02 | |
| GCG | other | 2.10E−02 | |
| TGFBR2 | kinase | 2.10E−02 | PN PTPN20 |
| Wnt | group | 2.11E−02 | |
| ADAM17 | peptidase | 2.11E−02 | |
| MGEA5 | enzyme | 2.14E−02 | RBB3 GABRA2 HMGCR HPGD SUCLG2 |
| losartan potassium | chemical drug | 2.15E−02 | |
| NFATC2 | transcription | 2.17E−02 | [symbol] C22D1 |
| | regulator | | |
| JUN | transcription | 2.18E−02 | [symbol] LMO1 MMP2 PTPRD SYK VAV3 VCAM1 |
| | regulator | | |
| EGR2 | transcription | 2.23E−02 | [symbol] L1 TNFSF11 |
| | regulator | | |
| mir-146 | microrna | 2.26E−02 | |
| S-nitrosoglutathione | chemical toxicant | 2.26E−02 | |
| DEF6 | other | 2.28E−02 | |
| TNFRSF6B | transmembrane | 2.28E−02 | |
| | receptor | | |
| ABCC8 | transporter | 2.28E−02 | |
| EFNB2 | other | 2.28E−02 | |
| IL1RL1 | transmembrane | 2.28E−02 | |
| | receptor | | |
| 2-acetylaminofluorene | chemical toxicant | 2.28E−02 | |
| allyl sulfide | chemical drug | 2.28E−02 | |
| TNFRSF1B | transmembrane | 2.30E−02 | |
| | receptor | | |
| cadmium | chemical toxicant | 2.30E−02 | |
| SPARC | other | 2.30E−02 | |
| ANGPT2 | growth factor | 2.33E−02 | SF11 VCAM1 |
| AHR | ligand-dependent | 2.35E−02 | G HMGCR ITGAM MMP2 NMT2 SOD1 |
| | nuclear receptor | | |
| PI3K (complex) | complex | 2.36E−02 | TPN12 SEMA6D SLC6A3 VCAM1 |
| TXNIP | other | 2.40E−02 | |
| 9 10-dimethyl-1 2-benzanthracene | chemical toxicant | 2.40E−02 | |
| acetaminophen | chemical drug | 2.43E−02 | 7B4 VCAM1 |
| Immunoglobulin | complex | 2.47E−02 | MMP2 VCAM1 |
| MITF | transcription | 2.47E−02 | SCAR PHACTR1 UVRAG |
| | regulator | | |
| poly rI:rC-RNA | biologic drug | 2.48E−02 | [symbol] G MMP2 NFKB1 STAM SUOX SYK |
| OSM | cytokine | 2.49E−02 | B3 FOXN3 GMPR IL15 LMO1 MMP2 |
| RET | kinase | 2.55E−02 | [symbol] |
| NPC1 | transporter | 2.56E−02 | |
| Ro31-8220 | chemical-kinase | 2.56E−02 | |
| | inhibitor | | |
| PECAM1 | other | 2.56E−02 | |
| WNT7A | cytokine | 2.56E−02 | |
| ACSL5 | enzyme | 2.56E−02 | |
| MUC4 | other | 2.56E−02 | |
| indirubin | chemical drug | 2.56E−02 | |
| metyrapone | chemical drug | 2.56E−02 | |
| EDN1 | cytokine | 2.57E−02 | [symbol] 1 VCAM1 |
| PTH | other | 2.58E−02 | [symbol] FSF11 |
| RORC | ligand-dependent | 2.58E−02 | UGGT2 |
| | nuclear receptor | | |
| bexarotene | chemical drug | 2.58E−02 | [symbol] AM1 |
| apigenin | chemical- | 2.62E−02 | |
| | endogenous non- | | |
| | mammalian | | |
| resveratrol | chemical drug | 2.66E−02 | PPARGC1A SOD1 SQSTM1 VCAM1 |
| KLF 15 | transcription | 2.72E−02 | |
| | regulator | | |
| cisplatin | chemical drug | 2.74E−02 | [symbol] B4 FOXN3 GPC5 HMGCR HYAL1 IL15 |
| | | | AM1 |
| CREBBP | transcription | 2.81E−02 | PKHD1 PPARGC1A RTP4 UTRN VCAM1 |
| | regulator | | |
| MAPK1 | kinase | 2.81E−02 | P2 PROS1 SUOX TNFSF11 TRIM5 |
| C5 | cytokine | 2.82E−02 | |
| LYL1 | transcription | 2.85E−02 | |
| | regulator | | |
| SIRT2 | transcription | 2.85E−02 | |
| | regulator | | |
| NFIA | transcription | 2.85E−02 | |
| | regulator | | |
| SLC6A3 | transporter | 2.85E−02 | |
| TNFRSF11A | transmembrane | 2.85E−02 | |
| | receptor | | |
| BID | other | 2.85E−02 | |
| NONO | other | 2.85E−02 | |
| dichlororibofuranosylbenzimidazole | chemical toxicant | 2.85E−02 | |
| docosahexaenoic acid | chemical drug | 2.88E−02 | CAM1 |
| BMI1 | transcription | 2.88E−02 | |
| | regulator | | |
| APOA1 | transporter | 2.88E−02 | |
| tegafur | chemical drug | 2.93E−02 | |
| ipilimumab | biologic drug | 2.93E−02 | |
| 2 2′ 4 4′-tetrabromodiphenyl ether | chemical reagent | 2.93E−02 | |
| DF 203 | chemical drug | 2.93E−02 | |
| Mitochondrial complex 1 | complex | 2.93E−02 | |
| NPS | other | 2.93E−02 | |
| des-Arg(10)-kallidin | chemical reagent | 2.93E−02 | |
| PLEKHM1 | other | 2.93E−02 | |
| NLRP10 | other | 2.93E−02 | |
| TAAR1 | g-protein coupled | 2.93E−02 | |
| | receptor | | |
| BTBD7 | other | 2.93E−02 | |
| GPR4 | g-protein coupled | 2.93E−02 | |
| | receptor | | |
| LRIG3 | other | 2.93E−02 | |
| EVX1 | transcription | 2.93E−02 | |
| | regulator | | |
| CBP-ICSBP-IRF-1-PU.1 | complex | 2.93E−02 | |
| ACAT1 | enzyme | 2.93E−02 | |
| FBLIM1 | other | 2.93E−02 | |
| RAP1B | enzyme | 2.93E−02 | |
| TRPC5 | ion channel | 2.93E−02 | |
| RBBP7 | transcription | 2.93E−02 | |
| | regulator | | |
| ATP1A1 | transporter | 2.93E−02 | |
| FAIM2 | other | 2.93E−02 | |
| IBSP | other | 2.93E−02 | |
| CCNC | other | 2.93E−02 | |
| HPGD | enzyme | 2.93E−02 | |
| CLCN3 | ion channel | 2.93E−02 | |
| mir-342 | microrna | 2.93E−02 | |
| mir-665 | microrna | 2.93E−02 | |
| GNAT1 | enzyme | 2.93E−02 | |
| EGFL7 | other | 2.93E−02 | |
| BAZ1A | other | 2.93E−02 | |
| PBK | kinase | 2.93E−02 | |
| GPR34 | g-protein coupled | 2.93E−02 | |
| | receptor | | |
| BCAN | other | 2.93E−02 | |
| LAMC2 | other | 2.93E−02 | |
| TXNDC5 | enzyme | 2.93E−02 | |
| RNF139 | enzyme | 2.93E−02 | |
| DRP2 | other | 2.93E−02 | |
| RHCE/RHD | transporter | 2.93E−02 | |
| cyclic des-acyl ghrelin (6-13) | biologic drug | 2.93E−02 | |
| PNRC1 | other | 2.93E−02 | |
| TRIP11 | transcription | 2.93E−02 | |
| | regulator | | |
| Sfl | other | 2.93E−02 | |
| gimeracil | chemical drug | 2.93E−02 | |
| phthalic acid | chemical- | 2.93E−02 | |
| | endogenous non- | | |
| | mammalian | | |
| palytoxin | chemical toxicant | 2.93E−02 | |
| alpha-endosulfan | chemical toxicant | 2.93E−02 | |
| primaquine | chemical drug | 2.93E−02 | |
| acetoxyacetylaminofluorene | chemical toxicant | 2.93E−02 | |
| gabazine | chemical drug | 2.93E−02 | |
| oxazepam | chemical drug | 2.93E−02 | |
| dibenzo(al)pyrene | chemical toxicant | 2.93E−02 | |
| benzo(a)pyrene-7 8-dione | chemical toxicant | 2.93E−02 | |
| procaine | chemical drug | 2.93E−02 | |
| lavendustin A | chemical-kinase | 2.93E−02 | |
| | inhibitor | | |
| all-trans-4-Oxo-retinoic acid | chemical- | 2.93E−02 | |
| | endogenous | | |
| | mammalian | | |
| indolo(3 2-b)carbazole | chemical reagent | 2.93E−02 | |
| linalool | chemical- | 2.93E−02 | |
| | endogenous non- | | |
| | mammalian | | |
| ginkgolide A | chemical- | 2.93E−02 | |
| | endogenous non- | | |
| | mammalian | | |
| KT 5926 | chemical-kinase | 2.93E−02 | |
| | inhibitor | | |
| sulfur | chemical- | 2.93E−02 | |
| | endogenous | | |
| | mammalian | | |
| trehalose dimycolate | chemical-other | 2.93E−02 | |
| elaidic acid | chemical- | 2.93E−02 | |
| | endogenous | | |
| | mammalian | | |
| epoxyeicosatrienoic acid | chemical-other | 2.93E−02 | |
| polyinosinic acid | chemical reagent | 2.93E−02 | |
| E64 | chemical- | 2.93E−02 | |
| | endogenous non- | | |
| | mammalian | | |
| 17-epiestriol | chemical- | 2.93E−02 | |
| | endogenous | | |
| | mammalian | | |
| 3 20-pregnanedione | chemical- | 2.93E−02 | |
| | endogenous | | |
| | mammalian | | |
| N-Ac-Leu-Leu-norleucinal | chemical-protease | 2.97E−02 | |
| | inhibitor | | |
| PROC | peptidase | 3.05E−02 | |
| FOXO1 | transcription | 3.07E−02 | YOU1 IL15 PPARGC1A SEMA6D VCAM1 |
| | regulator | | |
| IL33 | cytokine | 3.09E−02 | |
| SRC | kinase | 3.10E−02 | |
| benzyloxycarbonyl-Leu-Leu-Leu | chemical-protease | 3.16E−02 | [symbol] 2 SOD1 SQSTM1 VCAM1 |
| aldehyde | inhibitor | | |
| ADCY | group | 3.16E−02 | |
| Ncoa-Nrli2-Rxra | complex | 3.16E−02 | |
| NADPH oxidase | complex | 3.16E−02 | |
| Lymphotoxin | complex | 3.16E−02 | |
| CYP27A1 | enzyme | 3.16E−02 | |
| NRTN | growth factor | 3.16E−02 | |
| 1-methyl-4-phenylpyridinium | chemical toxicant | 3.16E−02 | |
| fumonisin B1 | chemical toxicant | 3.16E−02 | |
| ammonium chloride | chemical drug | 3.16E−02 | |
| leuprolide | biologic drug | 3.16E−02 | |
| STK11 | kinase | 3.20E−02 | H VAV3 |
| Alpha catenin | group | 3.20E−02 | |
| homocysteine | chemical- | 3.21E−02 | |
| | endogenous | | |
| | mammalian | | |
| TCR | complex | 3.22E−02 | NFKB1 TNFSF11 TSC22D1 |
| CHUK | kinase | 3.26E−02 | SQSTM1 VCAM1 |
| XRP1 | transcription | 3.26E−02 | SF11 VCAM1 |
| | regulator | | |
| androgen | chemical drug | 3.28E−02 | VAV3 |
| curcumin | chemical drug | 3.29E−02 | NFKB1 PPARGC1A SOD1 TNFSF11 |
| POU2F1 | transcription | 3.30E−02 | |
| | regulator | | |
| hydrogen peroxide | chemical- | 3.31E−02 | [symbol] MGCR MMP2 PPARGC1A SC5D SOD1 |
| | endogenous | | |
| | mammalian | | |
| NFYA | transcription | 3.34E−02 | |
| | regulator | | |
| TBK1 | kinase | 3.34E−02 | |
| NEkB (complex) | complex | 3.36E−02 | HIVEP1 ICOS IL15 ITGAM KRT6B MMP2 |
| TNFSF11 | cytokine | 3.38E−02 | [symbol] CAR TNFSF11 VCAM1 |
| LTBR | transmembrane | 3.40E−02 | |
| | receptor | | |
| ATG7 | enzyme | 3.40E−02 | |
| FOXC2 | transcription | 3.40E−02 | |
| | regulator | | |
| omeprazole | chemical drug | 3.40E−02 | |
| PML | transcription | 3.41E−02 | QSTM1 |
| | regulator | | |
| IRF7 | transcription | 3.46E−02 | 45 |
| | regulator | | |
| deferoxamine | chemical drug | 3.46E−02 | D1 VCAM1 |
| 11 12-epoxyeicosatrienoic acid | chemical- | 3.47E−02 | |
| | endogenous | | |
| | mammalian | | |
| puromycin | chemical- | 3.47E−02 | |
| | endogenous non- | | |
| | mammalian | | |
| HTATIP2 | transcription | 3.47E−02 | |
| | regulator | | |
| SIM2 | transcription | 3.47E−02 | |
| | regulator | | |
| EZR | other | 3.47E−02 | |
| F10 | peptidase | 3.47E−02 | |
| carvedilol | chemical drug | 3.47E−02 | |
| chelerythrine | chemical drug | 3.47E−02 | |
| BMP7 | growth factor | 3.51E−02 | [symbol] GC1A |
| Sb202190 | chemical-kinase | 3.51E−02 | [symbol] C1A |
| | inhibitor | | |
| CSF2 | cytokine | 3.55E−02 | L15 ITGAM ITGAX MMP2 NFKB1 |
| ELK1 | transcription | 3.58E−02 | |
| | regulator | | |
| ADM | other | 3.58E−02 | |
| streptozocin | chemical drug | 3.64E−02 | [symbol] C1A SLC6A3 SOD1 |
| PPARG | ligand-dependent | 3.66E−02 | CR HYOU1 PCTP PPARGC1A PPP2R2B |
| | nuclear receptor | | |
| tamoxifen | chemical drug | 3.66E−02 | [symbol] BB4 HMGCR SQSTM1 |
| IL10 | cytokine | 3.69E−02 | [symbol] GD MERTK MMP2 TNFSF11 VCAM1 |
| Ca2+ | chemical- | 3.73E−02 | OSCAR TCHH TNFSF11 |
| | endogenous | | |
| | mammalian | | |
| 5-O-mycolyl beta araf-(1->2)-5-O- | chemical- | 3.74E−02 | |
| mycolyl-alpha-araf-(1->1′)-glycerol | endogenous non- | | |
| | mammalian | | |
| cholecalciferol | chemical- | 3.74E−02 | |
| | endogenous | | |
| | mammalian | | |
| ADRA1A | g-protein coupled | 3.77E−02 | |
| | receptor | | |
| anisomycin | chemical- | 3.77E−02 | |
| | endogenous non- | | |
| | mammalian | | |
| JINK1/2 | group | 3.80E−02 | |
| NTN1 | other | 3.80E−02 | |
| PAX2 | transcription | 3.80E−02 | |
| | regulator | | |
| MECOM | transcription | 3.80E−02 | |
| | regulator | | |
| fluconazole | chemical drug | 3.80E−02 | |
| pifithrin alpha | chemical reagent | 3.80E−02 | |
| alendronic acid | chemical drug | 3.80E−02 | |
| probucol | chemical drug | 3.80E−02 | |
| cyclic AMP | chemical- | 3.88E−02 | IL15 PDE4D PPARGC1A |
| | endogenous | | |
| | mammalian | | |
| E. coli B5 lipopolysaccharide | chemical- | 3.88E−02 | PARGC1A SOD1 TNFSF11 |
| | endogenous non- | | |
| | mammalian | | |
| ERBB4 | kinase | 3.88E−02 | |
| INS | other | 3.95E−02 | [symbol] SOD1 |
| ZBTB20 | transcription | 3.90E−02 | |
| | regulator | | |
| vitamin D | chemical drug | 3.96E−02 | |
| N-nitro-L-arginine methyl ester | chemical drug | 4.02E−02 | |
| FOXO3 | transcription | 4.11E−02 | [symbol] 1A SOD1 |
| | regulator | | |
| thapsigargin | chemical toxicant | 4.11E−02 | VCAM1 |
| cycloheximide | chemical reagent | 4.13E−02 | IT4 DHRS2 ITGAM MMP2 TNFSF11 |
| CRTC2 | other | 4.14E−02 | |
| CABA | chemical- | 4.14E−02 | |
| | endogenous | | |
| | mammalian | | |
| selenomethylselenocysteine | chemical- | 4.14E−02 | |
| | endogenous | | |
| | mammalian | | |
| sucrose | chemical- | 4.14E−02 | |
| | endogenous | | |
| | mammalian | | |
| MAPKAPK2 | kinase | 4.16E−02 | |
| NKX3-1 | transcription | 4.16E−02 | |
| | regulator | | |
| NEKBIB | transcription | 4.16E−02 | |
| | regulator | | |
| DCN | other | 4.16E−02 | |
| raloxifene | chemical drug | 4.21E−02 | [symbol] FSF11 |
| PRKCD | kinase | 4.31E−02 | [symbol] M1 |
| Nfat (family) | group | 4.31E−02 | |
| IL12A | cytokine | 4.36E−02 | |
| TRAF6 | enzyme | 4.36E−02 | |
| CUX1 | transcription | 4.36E−02 | |
| | regulator | | |
| Go 6976 | chemical-kinase | 4.36E−02 | |
| | inhibitor | | |
| Rp-8-Br-cGMPS | chemical-kinase | 4.37E−02 | |
| | inhibitor | | |
| ergosterol-5 8-peroxide | chemical- | 4.37E−02 | |
| | endogenous non- | | |
| | mammalian | | |
| glycodeoxycholic acid | chemical- | 4.37E−02 | |
| | endogenous | | |
| | mammalian | | |
| taurochenodeoxycholate | chemical- | 4.37E−02 | |
| | endogenous | | |
| | mammalian | | |
| kawain | chemical- | 4.37E−02 | |
| | endogenous non- | | |
| | mammalian | | |
| benzo(a)pyrene 7 8-dihydrodiol | chemical toxicant | 4.37E−02 | |
| 14 15-DIIET | chemical- | 4.37E−02 | |
| | endogenous | | |
| | mammalian | | |
| methysticin | chemical reagent | 4.37E−02 | |
| 1 2 5 6-dibenzanthracene | chemical toxicant | 4.37E−02 | |
| diosmin | chemical drug | 4.37E−02 | |
| carfilzomib | biologic drug | 4.37E−02 | |
| fluoroquinolone | chemical drug | 4.37E−02 | |
| prochloraz | chemical reagent | 4.37E−02 | |
| CpG ODN 1555 | chemical reagent | 4.37E−02 | |
| dietary fat derivative | chemical drug | 4.37E−02 | |
| ATP6AP2 | transporter | 4.37E−02 | |
| patulin | chemical toxicant | 4.37E−02 | |
| TMBIM1 | other | 4.37E−02 | |
| PIH1D3 | other | 4.37E−02 | |
| GLDN | other | 4.37E−02 | |
| FREM1 | other | 4.37E−02 | |
| ZXDC | transcription | 4.37E−02 | |
| | regulator | | |
| GALNT6 | enzyme | 4.37E−02 | |
| SPON2 | other | 4.37E−02 | |
| DERL1 | other | 4.37E−02 | |
| FREM2 | other | 4.37E−02 | |
| Integrin alpha 3 beta 1 | complex | 4.37E−02 | |
| ITGBL1 | other | 4.37E−02 | |
| P2RY6 | g-protein coupled | 4.37E−02 | |
| | receptor | | |
| PTPN23 | phosphatase | 4.37E−02 | |
| Ap1 gamma | group | 4.37E−02 | |
| Eotaxin | group | 4.37E−02 | |
| ADRBK2 | kinase | 4.37E−02 | |
| RPL13A | other | 4.37E−02 | |
| NRCAM | other | 4.37E−02 | |
| BPTF | transcription | 4.37E−02 | |
| | regulator | | |
| HAO1 | enzyme | 4.37E−02 | |
| mir-541 | microrna | 4.37E−02 | |
| miR-485-3p (and other miRNAs | mature microrna | 4.37E−02 | |
| w/seed UCAUACA) | | | |
| PHGDH | enzyme | 4.37E−02 | |
| VSX2 | transcription | 4.37E−02 | |
| | regulator | | |
| SLN | other | 4.37E−02 | |
| PFN1 | other | 4.37E−02 | |
| TCF20 | transcription | 4.37E−02 | |
| | regulator | | |
| GFRA2 | transmembrane | 4.37E−02 | |
| | receptor | | |
| ADRA2A | g-protein coupled | 4.37E−02 | |
| | receptor | | |
| GNG2 | enzyme | 4.37E−02 | |
| ANK1 | other | 4.37E−02 | |
| NPHS2 | other | 4.37E−02 | |
| TFPI2 | other | 4.37E−02 | |
| ITGAD | other | 4.37E−02 | |
| DHCR7 | enzyme | 4.37E−02 | |
| AHCY | enzyme | 4.37E−02 | |
| NR2F6 | ligand-dependent | 4.37E−02 | |
| | nuclear receptor | | |
| KLF16 | other | 4.37E−02 | |
| DNM2 | enzyme | 4.37E−02 | |
| (E)-1-(2-nitrovinyl)naphthalene | chemical reagent | 4.37E−02 | |
| (E)-9-(2-nitrovinyl)anthracene | chemical reagent | 4.37E−02 | |
| N-butyryl arginine | chemical reagent | 4.37E−02 | |
| 3-hydroxybutyrate arginate | chemical reagent | 4.37E−02 | |
| phenylacetic acid | chemical- | 4.37E−02 | |
| | endogenous | | |
| | mammalian | | |
| anastrozole | chemical drug | 4.37E−02 | |
| nilutamide | chemical drug | 4.37E−02 | |
| dichlorodiphenyl dichloroethylene | chemical toxicant | 4.37E−02 | |
| amantadine | chemical drug | 4.37E−02 | |
| PTIO | chemical reagent | 4.37E−02 | |
| oxonic acid | chemical drug | 4.37E−02 | |
| bongkrekic acid | chemical toxicant | 4.37E−02 | |
| trimetazidine | chemical drug | 4.37E−02 | |
| apomine | chemical drug | 4.37E−02 | |
| ropivacaine | chemical drug | 4.37E−02 | |
| 3-nitrobenzanthrone | chemical toxicant | 4.37E−02 | |
| myristoylated PKCzeta | chemical-kinase | 4.37E−02 | |
| pseudosubstrate peptide inhibitor | inhibitor | | |
| (Ala9)-autocamtide-2 | chemical-kinase | 4.37E−02 | |
| | inhibitor | | |
| indole | chemical- | 4.37E−02 | |
| | endogenous | | |
| | mammalian | | |
| zeaxanthin | chemical drug | 4.37E−02 | |
| acacetin | chemical- | 4.37E−02 | |
| | endogenous non- | | |
| | mammalian | | |
| taxifolin | chemical- | 4.37E−02 | |
| | endogenous non- | | |
| | mammalian | | |
| physostigmine | chemical drug | 4.37E−02 | |
| latanoprost | chemical drug | 4.37E−02 | |
| glyphosate | chemical toxicant | 4.37E−02 | |
| arginine butyrate | chemical drug | 4.37E−02 | |
| chitosan | chemical- | 4.37E−02 | |
| | endogenous non- | | |
| | mammalian | | |
| megestrol acetate | chemical drug | 4.37E−02 | |
| LDLR | transporter | 4.46E−02 | |
| Ikb | group | 4.50E−02 | |
| MTDH | transcription | 4.50E−02 | |
| | regulator | | |
| AKT3 | kinase | 4.50E−02 | |
| LCAT | enzyme | 4.50E−02 | |
| RC3H1 | enzyme | 4.50E−02 | |
| KN-62 | chemical-kinase | 4.50E−02 | |
| | inhibitor | | |
| evodiamine | chemical- | 4.50E−02 | |
| | endogenous non- | | |
| | mammalian | | |
| RARA | ligand-dependent | 4.50E−02 | KLF3-AS1 RTP4 XK |
| | nuclear receptor | | |
| glucagon | biologic drug | 4.57E−02 | |
| luteolin | chemical- | 4.57E−02 | |
| | endogenous non- | | |
| | mammalian | | |
| ASCL1 | transcription | 4.61E−02 | |
| | regulator | | |
| SAMSN1 | other | 4.61E−02 | |
| 8-bromo-cAMP | chemical reagent | 4.62E−02 | PPARGC1A |
| FOXA1 | transcription | 4.66E−02 | |
| | regulator | | |
| FAS | transmembrane | 4.74E−02 | ERTK PTPRN2 SYK TLN2 VCAM1 |
| | receptor | | |
| PRL | cytokine | 4.75E−02 | DPN SOD1 TNFSF11 |
| miR-141-3p (and other miRNAs | mature microrna | 4.78E−02 | |
| w/seed AACACUG) | | | |
| SERPINF1 | other | 4.78E−02 | |
| BIRC5 | other | 4.78E−02 | |
| thioctic acid | chemical drug | 4.78E−02 | |
| SPP1 | cytokine | 4.83E−02 | [symbol] FSF11 |
| BMP | group | 4.86E−02 | |
| BGN | other | 4.86E−02 | |
| ICOSLG/LOC102723996 | other | 4.86E−02 | |
| PKM | kinase | 4.86E−02 | |
| ITGA1 | other | 4.86E−02 | |
| DPP4 | peptidase | 4.86E−02 | |
| TRPV4 | ion channel | 4.86E−02 | |
| HSPD1 | enzyme | 4.86E−02 | |
| MED1 | transcription | 4.86E−02 | |
| | regulator | | |
| methyl-beta-cyclodextrin | chemical drug | 4.86E−02 | |
| NOTCH1 | transcription | 4.86E−02 | [symbol] K TNFSF11 |
| | regulator | | |
| tetrachlorodibenzodioxin | chemical toxicant | 4.88E−02 | [symbol] B3 ESRRG ITGAX NMT2 SLCO3A1 SOD1 |
| Pka | complex | 4.91E−02 | [symbol] F11 |
| TNFSF10 | cytokine | 4.93E−02 | |

^{A}shows the upstream regulators;
^{B}shows whether the upstream regulator is a chemical drug, transcription regulator among others;
^{C}shows the significance of the role of this regulator in gene regulation;
^{D}shows the target genes of the upstream regulator which are also the target genes of the human CRISPRs. [symbol]
indicates data missing or illegible when filed

To further analyse the human CRISPR controlled gene regulation during different (a)biotic conditions a microarray platform the Human Genome U133 Plus 2.0 Array for Affymetrix was explored. The probe sequences and annotation of this Affymetrix chip were downloaded and then uploaded into Galaxy software analyses tool and transformed into a Blast database using a standard software package in Galaxy (https://bioinf-galaxian.erasmusmc.nl/galaxy/). Secondly, the human CRISPR sequences were separated in a group containing only the spacer sequences and in a group containing only the repeat sequences. These were also uploaded into the Galaxy software analysis tool. After BLAST analyses we retrieved a significant hit with a human CRISPR spacer sequence for more than 2000 probes of the Human Genome U133 Plus 2.0 Array (Table 6).

TABLE 6
Affymetrix probes harbor significant identity with the human
CRISPR spacers.

| SEQ ID NO | Probe Set ID | E-Value | Gene Target Symbol^{E} |
|---|---|---|---|
| 5927 | 1562661_at | 1.00E−06 | — |
| 4064 | 239410_at | 1.75E−06 | — |
| 7250 | 210230_at | 5.00E−08 | — |
| 9629 | 234590_x_at | 2.00E−07 | — |
| 4599 | 238016_s_at | 4.00E−08 | — |
| 6399 | 232273_at | 4.00E−08 | — |
| 8195 | 230580_at | 8.10E−07 | — |
| 2750 | 1569527_at | 5.00E−07 | — |
| 5856 | 1566501_at | 1.00E−06 | — |
| 3595 | 1557580_at | 8.00E−08 | — |
| 9831 | 234753_x_at | 5.00E−08 | — |
| 3020 | 1563721_at | 1.00E−06 | — |
| 4331 | 205183_at | 2.00E−07 | — |
| 8960 | 1560118_at | 6.00E−09 | — |
| 3595 | 1566541_at | 3.01E−08 | — |
| 8384 | 216692_at | 5.00E−07 | — |
| 1038 | 1554963_at | 4.50E−07 | — |
| 185 | 1569182_at | 2.80E−07 | — |
| 6399 | 243768_at | 5.00E−07 | — |
| 1038 | 1554964_x_at | 4.50E−07 | — |
| 8678 | 230461_s_at | 5.00E−21 | — |
| 8195 | 215958_at | 3.00E−07 | — |
| 4331 | 1557335_at | 5.00E−08 | — |
| 5856 | 234247_at | 4.00E−08 | — |
| 2700 | 1556521_a_at | 4.00E−12 | — |
| 3395 | 217482_at | 1.40E−06 | — |
| 63 | 1565857_at | 5.00E−09 | — |
| 7988 | 1557774_at | 2.36E−08 | — |
| 3595 | 1557581_x_at | 8.00E−08 | — |
| 7665 | 234574_at | 3.00E−10 | — |
| 8195 | 224086_at | 2.70E−07 | — |
| 5927 | 241867_at | 2.50E−07 | — |
| 4475 | 1559434_at | 8.00E−08 | — |
| 6860 | 237187_at | 1.00E−21 | — |
| 5927 | 1558889_at | 5.75E−07 | — |
| 4064 | 1569416_at | 1.25E−07 | — |
| 4865 | 242495_at | 1.50E−07 | — |
| 8823 | 220874_at | 8.00E−09 | — |
| 4331 | 238812_at | 2.00E−06 | — |
| 8195 | 238301_at | 1.00E−08 | — |
| 4331 | 242256_x_at | 2.00E−07 | — |
| 7461 | 216767_at | 4.00E−07 | — |
| 2836 | 1562642_at | 3.00E−13 | — |
| 6113 | 1566425_at | 1.00E−22 | — |
| 250 | 1569603_at | 4.00E−07 | — |
| 6009 | 1562553_at | 3.00E−08 | — |
| 8407 | 241468_at | 1.00E−12 | — |
| 676 | 233235_x_at | 5.00E−07 | — |
| 9005 | 234654_at | 1.00E−13 | — |
| 9101 | 1562586_at | 3.00E−15 | — |
| 8198 | 1560230_at | 2.00E−07 | — |
| 4064 | 1561737_at | 1.00E−09 | — |
| 6914 | 1564984_at | 4.00E−11 | — |
| 8792 | 242795_at | 1.00E−11 | — |
| 7129 | 1555664_at | 9.00E−10 | — |
| 8195 | 215928_at | 1.00E−08 | — |
| 9342 | 242924_at | 9.00E−25 | — |
| 5170 | 1561642_at | 4.00E−07 | — |
| 8960 | 1559724_at | 2.00E−09 | — |
| 558 | 233716_at | 1.00E−04 | — |
| 559 | 217052_x_at | 0.002034 | — |
| 581 | 221788_at | 0.005 | PGM3 |
| 592 | 227931_at | 0.001 | INO80D |
| 592 | 231798_at | 0.004 | NOG |
| 639 | 205481_at | 6.00E−19 | ADORA1 |
| 641 | 1555433_at | 6.00E−06 | SLC39A14 |
| 653 | 234659_at | 3.00E−04 | — |
| 660 | 1569768_at | 1.00E−04 | — |
| 660 | 1569770_x_at | 0.001319 | — |
| 662 | 244172_at | 5.40E−04 | — |
| 662 | 235662_at | 0.004 | — |
| 662 | 224322_at | 0.004 | ARID4B |
| 662 | 215102_at | 0.002 | DPY19L1P1 |
| 669 | 235175_at | 2.48E−04 | GBP4 |
| 669 | 219724_s_at | 2.98E−04 | TESPA1 |
| 676 | 216189_at | 8.00E−05 | — |
| 676 | 232441_at | 2.00E−06 | KRR1 |
| 676 | 1560119_at | 7.00E−06 | LOC389634 |
| 676 | 202707_at | 1.54E−04 | UMPS |
| 683 | 227173_s_at | 0.004 | BACH2 |
| 687 | 233715_at | 0.002 | — |
| 687 | 1559026_at | 5.00E−05 | LOC100506599 |
| 720 | 215607_x_at | 5.10E−04 | — |
| 779 | 217704_x_at | 0.001505 | SUZ12P |
| 796 | 1559125_at | 7.00E−06 | — |
| 804 | 236247_at | 0.004 | NSUN4 |
| 807 | 1563074_at | 1.00E−05 | LOC255654 |
| 5679 | 207235_s_at | 0.005 | GRM5 |
| 6075 | 235582_at | 0.003 | E2F2 |
| 6092 | 213897_s_at | 1.00E−13 | MRPL23 |
| 5711 | 1564212_at | 0.001 | — |
| 6105 | 39763_at | 1.00E−12 | HPX |
| 6131 | 1559530_at | 4.00E−06 | — |
| 6131 | 240025_x_at | 1.00E−05 | — |
| 6131 | 215763_at | 5.00E−04 | — |
| 6131 | 231843_at | 4.05E−04 | DDX55 |
| 6131 | 210005_at | 6.00E−04 | GART |
| 6131 | 236255_at | 6.00E−04 | PLEKHG4B |
| 6131 | 204477_at | 6.00E−04 | RABIF |
| 6131 | 233321_x_at | 5.00E−05 | LOC90834 |
| 6131 | 222196_at | 4.05E−04 | LOC389906 |
| 5717 | 232875_at | 5.40E−04 | — |
| 5717 | 242025_at | 0.001 | — |
| 6194 | 241924_at | 0.001 | MECP2 |
| 6240 | 242526_at | 3.00E−04 | — |
| 6240 | 225690_at | 3.00E−04 | CDK12 |
| 6240 | 218988_at | 8.00E−05 | SLC35E3 |
| 6240 | 1559870_at | 2.00E−05 | LOC100129129 |
| 6249 | 1568864_at | 5.56E−04 | LOC100131691 |
| 6251 | 1560542_at | 5.00E−06 | MCM3AP-AS1 |
| 6266 | 1558315_s_at | 2.70E−05 | HOOK3 |
| 6266 | 220934_s_at | 0.003 | TMEM223 |
| 6267 | 201800_s_at | 0.001 | OSBP |
| 5732 | 231886_at | 3.83E−04 | LOC100134822 |
| 6310 | 216165_at | 3.00E−04 | — |
| 6310 | 208233_at | 1.00E−04 | PDPN |
| 6323 | 236854_at | 0.004 | LINC00494 |
| 5736 | 221164_x_at | 3.75E−06 | CHST5 |
| 6365 | 232790_at | 2.00E−07 | LOC100506054 |
| 6375 | 220335_x_at | 4.62E−04 | CES3 |
| 6381 | 1560540_x_at | 7.00E−06 | — |
| 6399 | 234492_at | 7.00E−06 | — |
| 6399 | 1565802_at | 0.001 | — |
| 6399 | 209527_at | 3.00E−04 | EXOSC2 |
| 6399 | 1566140_at | 2.00E−06 | HOPX |
| 6399 | 242377_x_at | 3.65E−06 | THUMPD3 |
| 6399 | 240617_at | 3.00E−04 | LOC100287098 |
| 6399 | 239083_at | 2.00E−07 | ZNF786 |
| 6411 | 232910_at | 7.00E−05 | BBIP1 |
| 6411 | 216424_at | 0.001433 | CD4 |
| 6416 | 1557590_at | 6.00E−04 | PARD6G-AS1 |
| 6430 | 239603_x_at | 9.00E−05 | — |
| 6430 | 233315_at | 6.50E−04 | — |
| 6430 | 241686_x_at | 0.001024 | — |
| 6430 | 234565_x_at | 0.001335 | — |
| 6430 | 1570065_at | 0.001733 | — |
| 6430 | 1561346_at | 0.0025 | — |
| 6430 | 222961_at | 0.0035 | — |
| 6430 | 1557478_at | 0.0035 | — |
| 6430 | 234423_x_at | 0.004 | — |
| 6430 | 1560781_at | 3.43E−04 | DNIID1 |
| 6430 | 224275_at | 0.001334 | GPR98 |
| 6430 | 232703_at | 0.001 | GLUD1P7 |
| 6430 | 218637_at | 0.0035 | IMPACT |
| 6430 | 1553265_at | 0.0035 | SLC23A3 |
| 6430 | 234697_x_at | 0.004 | TAMM41 |
| 6430 | 1558253_x_at | 9.67E−04 | ZNF587 /// ZNF587B |
| 6430 | 1558251_a_at | 9.67E−04 | ZNF587 /// ZNF587B |
| 6431 | 1569409_x_at | 7.03E−04 | — |
| 6431 | 215387_x_at | 8.11E−04 | — |
| 6431 | 207860_at | 1.00E−04 | NCR1 |
| 6431 | 206482_at | 1.80E−04 | PTK6 |
| 6431 | 231938_at | 4.07E−04 | SGOL1 |
| 6431 | 1555363_s_at | 1.10E−07 | LOC284440 |
| 10004 | 230063_at | 9.00E−05 | ZNF264 |
| 6515 | 1564413_at | 5.48E−04 | FLJ36116 |
| 6583 | 244869_at | 1.00E−04 | — |
| 6583 | 224667_x_at | 1.00E−04 | ANAPC16 |
| 6583 | 215058_at | 0.001 | DENND5B |
| 6619 | 1553882_at | 8.00E−04 | — |
| 6634 | 215255_at | 0.001502 | IGSF9B |
| 6647 | 240698_s_at | 2.00E−04 | — |
| 6647 | 239790_s_at | 2.00E−04 | — |
| 6647 | 241737_x_at | 2.57E−04 | — |
| 6647 | 1570308_at | 3.47E−04 | — |
| 6647 | 239817_at | 5.10E−04 | — |
| 6647 | 1563210_at | 0.004 | — |
| 6647 | 1568850_at | 7.33E−05 | CCBP2 |
| 6647 | 214666_x_at | 4.28E−04 | IREB2 |
| 6647 | 206370_at | 0.003 | PIK3CG |
| 6647 | 214750_at | 2.00E−04 | PLAC4 |
| 6648 | 242321_at | 5.00E−05 | PTPN14 |
| 6661 | 1558275_at | 1.80E−04 | — |
| 6661 | 215893_x_at | 3.36E−04 | — |
| 6661 | 242609_x_at | 5.00E−04 | — |
| 6661 | 217639_at | 5.00E−04 | — |
| 6661 | 233627_at | 7.18E−04 | — |
| 6661 | 234656_x_at | 7.80E−04 | — |
| 6661 | 1569474_at | 8.02E−04 | — |
| 6661 | 215604_x_at | 8.70E−04 | — |
| 6661 | 1556931_at | 0.001251 | — |
| 6661 | 234494_x_at | 0.00165 | — |
| 6661 | 215349_at | 7.63E−04 | BTBD18 |
| 6661 | 1555346_at | 1.26E−04 | CDC20B |
| 6661 | 232065_x_at | 8.04E−04 | CENPL |
| 6661 | 242649_x_at | 3.10E−04 | HMGN2P46 |
| 6661 | 215112_x_at | 6.17E−04 | MCF2L2 |
| 6661 | 215067_x_at | 6.52E−04 | PRDX2 |
| 6661 | 1560770_at | 5.51E−04 | PABPC1 |
| 6661 | 233852_at | 4.16E−05 | POLH |
| 6661 | 1560445_x_at | 0.0015 | ARHGEF1 |
| 6661 | 227196_at | 0.001077 | RHPN2 |
| 6661 | 232392_at | 5.02E−05 | SRSF3 |
| 6661 | 213936_x_at | 5.01E−04 | SFTPB |
| 6661 | 237469_at | 0.0025 | TOP2A |
| 6661 | 1562566_at | 0.001427 | TRAF3IP2-AS1 |
| 6661 | 203148_s_at | 9.38E−04 | TRIM14 |
| 6661 | 1555322_at | 0.001174 | PP2672 |
| 6661 | 223845_at | 0.001333 | VSIG10 |
| 6672 | 216425_at | 0.00204 | — |
| 6672 | 208154_at | 0.004 | — |
| 6672 | 242265_at | 0.00235 | BRD8 |
| 6672 | 233649_at | 3.64E−04 | KATNAL2 |
| 6674 | 1560511_at | 0.003 | — |
| 6713 | 229711_s_at | 5.00E−10 | MDM2 |
| 6727 | 1560721_at | 0.001467 | — |
| 6739 | 233644_at | 7.67E−04 | KATNAL2 |
| 6747 | 225864_at | 0.003 | FAM84B |
| New 11 | 215628_x_at | 7.30E−04 | — |
| 6786 | 233554_at | 2.00E−06 | — |
| 6786 | 1563026_at | 0.001 | — |
| 6787 | 227683_x_at | 3.00E−07 | NUDT4 |
| 6807 | 206876_at | 0.002 | SIM1 |
| 6811 | 233431_x_at | 1.33E−05 | — |
| 6811 | 1556658_a_at | 1.69E−04 | — |
| 6811 | 232495_x_at | 1.90E−04 | — |
| 6811 | 211411_at | 2.32E−04 | — |
| 6811 | 1566538_at | 2.49E−04 | — |
| 6811 | 1562823_at | 2.61E−04 | — |
| 6811 | 1565821_at | 2.64E−04 | — |
| 6811 | 1565820_x_at | 2.64E−04 | — |
| 6811 | 1565819_at | 2.64E−04 | — |
| 6811 | 208082_x_at | 2.89E−04 | — |
| 6811 | 222319_at | 3.00E−04 | — |
| 6811 | 215868_x_at | 3.29E−04 | — |
| 6811 | 234578_at | 3.40E−04 | — |
| 6811 | 215862_at | 4.03E−04 | — |
| 6811 | 211444_at | 4.62E−04 | — |
| 6811 | 232580_x_at | 5.76E−04 | — |
| 6811 | 217571_at | 5.94E−04 | — |
| 6811 | 1569577_x_at | 6.26E−04 | — |
| 6811 | 1552975_x_at | 6.57E−04 | — |
| 6811 | 1552974_at | 6.57E−04 | — |
| 6811 | 220700_at | 6.88E−04 | — |
| 6811 | 1562063_x_at | 6.91E−04 | — |
| 6811 | 1566953_x_at | 7.04E−04 | — |
| 6811 | 1566952_at | 7.04E−04 | — |
| 6811 | 1558672_at | 7.18E−04 | — |
| 6811 | 1562062_at | 7.52E−04 | — |
| 6811 | 222295_x_at | 8.26E−04 | — |
| 6811 | 239333_x_at | 8.49E−04 | — |
| 6811 | 233900_at | 8.71E−04 | — |
| 6811 | 216229_x_at | 0.001001 | — |
| 6811 | 217703_x_at | 0.001049 | — |
| 6811 | 1561966_at | 0.001086 | — |
| 6811 | 216043_x_at | 0.0012 | — |
| 6811 | 215956_at | 0.001401 | — |
| 6811 | 217524_x_at | 0.002053 | — |
| 6811 | 216197_at | 6.70E−04 | ATF7IP |
| 6811 | 235049_at | 1.72E−04 | ADCY1 |
| 6811 | 1555369_at | 7.45E−04 | BAGE |
| 6811 | 202901_x_at | 4.52E−04 | CTSS |
| 6811 | 215377_at | 2.41E−05 | CTBP2 |
| 6811 | 1562772_a_at | 2.83E−04 | DAND5 |
| 6811 | 218362_s_at | 8.80E−04 | DIS3 |
| 6811 | 211129_x_at | 0.001024 | EDA |
| 6811 | 211130_x_at | 0.001024 | EDA |
| 6811 | 211127_x_at | 0.001024 | EDA |
| 6811 | 210525_x_at | 0.001019 | EFCAB11 |
| 6811 | 207822_at | 9.89E−04 | FGFR1 |
| 6811 | 232905_at | 0.001636 | GTF2H5 |
| 6811 | 1560396_at | 3.60E−04 | KLHL6 |
| 6811 | 223350_x_at | 2.21E−04 | LIN7C |
| 6811 | 220904_at | 4.72E−04 | LINC00574 |
| 6811 | 217456_x_at | 2.05E−04 | HLA-E |
| 6811 | 210869_s_at | 1.22E−04 | MCAM |
| 6811 | 1553688_at | 1.04E−04 | METTL6 |
| 6811 | 1553407_at | 4.91E−04 | MACF1 |
| 6811 | 215582_x_at | 2.41E−04 | MCM3AP |
| 6811 | 239268_at | 8.96E−04 | NDUFS1 |
| 6811 | 235432_at | 5.92E−04 | NPHP3 |
| 6811 | 1557172_x_at | 9.17E−04 | NEK8 |
| 6811 | 204185_x_at | 0.001113 | PPID |
| 6811 | 202122_s_at | 2.03E−04 | PLIN3 |
| 6811 | 205194_at | 1.47E−04 | PSPH |
| 6811 | 205233_s_at | 1.09E−04 | PAFAH2 |
| 6811 | 231809_x_at | 1.58E−04 | PDCD7 |
| 6811 | 1560715_at | 7.72E−04 | P4HA3 |
| 6811 | 232161_x_at | 5.42E−04 | PTPN4 |
| 6811 | 203735_x_at | 1.62E−04 | PPFIBP1 |
| 6811 | 217201_at | 2.27E−04 | RASAL2 |
| 6811 | 216247_at | 0.001225 | RPS20 /// SNORD54 |
| 6811 | 215856_at | 2.46E−04 | SIGLEC15 |
| 6811 | 218392_x_at | 2.05E−04 | SFXN1 |
| 6811 | 1560741_at | 6.90E−04 | SNRPN /// SNURF |
| 6811 | 224029_x_at | 1.37E−04 | SCN11A |
| 6811 | 1562415_a_at | 1.07E−04 | SPOCD1 |
| 6811 | 235526_at | 5.33E−04 | SOX6 |
| 6811 | 1554686_at | 2.43E−04 | STAU2 |
| 6811 | 215673_at | 2.15E−04 | TEF |
| 6811 | 219206_x_at | 8.04E−05 | TMBIM4 |
| 6811 | 222252_x_at | 1.32E−04 | UBQLN4 |
| 6811 | 221477_s_at | 6.52E−04 | LOC100129518 /// SOD2 |
| 6811 | 233840_at | 6.04E−04 | LOC100130950 |
| 6811 | 233775_x_at | 4.52E−04 | LOC100289333 |
| 6811 | 1562365_at | 0.001093 | LOC286177 |
| 6811 | 233210_at | 1.39E−04 | FLJ12120 |
| 6811 | 220020_at | 0.001426 | XPNPEP3 |
| 6811 | 231848_x_at | 6.03E−04 | ZNF207 |
| 6811 | 231988_x_at | 4.77E−04 | ZNF490 |
| 6811 | 219741_x_at | 5.50E−04 | ZNF552 |
| 6811 | 206180_x_at | 8.51E−04 | ZNF747 |
| 6811 | 219782_s_at | 5.82E−04 | ZNF771 |
| 6811 | 215826_x_at | 7.47E−04 | ZNF835 |
| 6838 | 243751_at | 7.50E−04 | CHD2 |
| 6859 | 1566485_at | 3.34E−04 | — |
| 6859 | 233994_at | 7.00E−04 | — |
| 6859 | 1566481_at | 0.001 | — |
| 6859 | 1563012_x_at | 0.001 | — |
| 6859 | 1561896_at | 0.001 | — |
| 6859 | 234788_x_at | 0.00201 | — |
| 6859 | 216601_at | 9.21E−04 | AOC4 |
| 6859 | 234981_x_at | 0.004 | CMBL |
| 6859 | 1553871_at | 8.00E−06 | CSNK1G2-AS1 |
| 6859 | 223487_x_at | 0.001 | GNB4 |
| 6859 | 244616_x_at | 2.76E−04 | MDM2 |
| 6859 | 204387_x_at | 0.002 | MRP63 |
| 6859 | 231437_at | 3.00E−04 | SLC35D2 |
| 6859 | 233399_x_at | 0.001 | TMED10P1 /// ZNF252 |
| 6859 | 214871_x_at | 0.001101 | LOC100287590 |
| 6859 | 1557472_a_at | 3.80E−04 | FLJ30838 |
| 6859 | 1562434_at | 1.00E−04 | UNK |
| 6878 | 238611_at | 0.001 | — |
| 6878 | 237549_at | 0.001 | — |
| 6878 | 236215_at | 0.001 | — |
| 6878 | 233817_at | 0.001 | — |
| 6878 | 233243_at | 0.001 | — |
| 6878 | 1569916_at | 0.001 | — |
| 6878 | 1569703_a_at | 0.001 | — |
| 6878 | 1569167_at | 0.001 | — |
| 6878 | 241207_at | 0.003 | — |
| 6878 | 217549_at | 0.003 | — |
| 6878 | 216110_x_at | 0.003 | — |
| 6878 | 242515_x_at | 0.001575 | AKIP1 |
| 6878 | 226036_x_at | 0.001 | CASP2 |
| 6878 | 224278_at | 0.001 | CYP4F30P |
| 6878 | 241923_x_at | 0.001 | NANS |
| 6878 | 206285_at | 6.00E−04 | NPHP1 |
| 6878 | 243643_x_at | 0.001 | SLC30A6 |
| 6878 | 1555259_at | 0.001 | ZAK |
| 6878 | 221085_at | 5.01E−04 | TNFSF15 |
| 6878 | 226891_at | 0.001 | XXYLT1 |
| 6935 | 216214_at | 5.00E−04 | — |
| 6943 | 1555510_at | 2.07E−04 | ZNF215 |
| 6953 | 232244_at | 2.00E−06 | KIAA1161 |
| 5803 | 209613_s_at | 0.002 | ADH1B |
| 5803 | 215046_at | 2.00E−04 | KANSL1L |
| 5803 | 206868_at | 2.00E−04 | STARD8 |
| 7004 | 1552311_a_at | 9.32E−04 | RAX2 |
| 7009 | 1558919_a_at | 3.00E−04 | SEC23IP |
| 7020 | 227040_at | 3.00E−16 | NHLRC3 |
| 7033 | 216739_at | 3.00E−04 | — |
| 7153 | 220583_at | 5.02E−04 | — |
| 7153 | 213759_at | 0.001 | ARL4C |
| 7153 | 229400_at | 0.001 | HOXD10 |
| 7153 | 218418_s_at | 0.001 | KANK2 |
| 7153 | 230863_at | 0.001 | LRP2 |
| 7153 | 1563458_at | 0.001 | PARVA |
| 7153 | 220025_at | 0.001 | TBR1 |
| 7153 | 1559756_at | 0.001 | DKFZp667F0711 |
| 5607 | 206825_at | 7.00E−04 | OXTR |
| 7212 | 241836_x_at | 0.00265 | CEP97 |
| 7257 | 233310_at | 5.00E−05 | — |
| 7267 | 220296_at | 8.00E−06 | — |
| 7300 | 210769_at | 0.003 | CNGB1 |
| 7300 | 1553389_at | 5.71E−04 | NPHP3 |
| 5837 | 1558976_x_at | 3.28E−04 | FAM100A |
| 5837 | 1558975_at | 3.28E−04 | FAM100A |
| 7313 | 213388_at | 0.0021 | LOC728802 /// PDE4DIP |
| 5839 | 222929_at | 3.00E−04 | FAM160B2 |
| 5839 | 222928_s_at | 3.00E−04 | FAM160B2 |
| 5839 | 1556178_x_at | 7.53E−05 | TAF8 |
| 5839 | 1559180_at | 0.001 | LOC100131860 |
| 7331 | 215200_x_at | 3.55E−04 | — |
| 7331 | 220252_x_at | 3.31E−04 | CXorf21 |
| 7331 | 235327_x_at | 0.001418 | UBXN2A |
| 5840 | 1562076_at | 3.60E−04 | — |
| 5840 | 1569912_at | 4.00E−04 | — |
| 5840 | 212886_at | 1.24E−04 | CCDC69 |
| 5840 | 215308_at | 0.001 | XRCC6 |
| 7362 | 232251_at | 2.00E−05 | NUDT16P1 |
| 7397 | 205101_at | 0.0015 | CIITA |
| 7421 | 1559843_s_at | 4.50E−05 | — |
| 7421 | 206091_at | 4.50E−05 | MATN3 |
| 7453 | 236921_at | 8.00E−05 | — |
| 7461 | 1564674_a_at | 9.35E−05 | CDC20B |
| 7461 | 240111_at | 0.0015 | RHOBTB3 |
| 7468 | 1564905_at | 2.00E−05 | — |
| 7468 | 215645_at | 0.0016 | FLCN |
| 7468 | 233812_at | 9.00E−04 | LINC00028 |
| 7468 | 215066_at | 0.001667 | PTPRF |
| 5856 | 215219_at | 1.00E−06 | DOPEY2 |
| 7481 | 1552677_a_at | 0.002 | DIP2A |
| 7481 | 1561482_at | 0.002 | LOC100133308 |
| 7536 | 1562644_at | 3.36E−04 | MTHFD2L |
| 7542 | 216151_at | 1.60E−04 | — |
| 7542 | 1569231_x_at | 1.60E−04 | — |
| 7542 | 1569230_at | 1.60E−04 | — |
| 7542 | 244838_at | 3.00E−04 | — |
| 7542 | 216613_at | 3.00E−04 | — |
| 7542 | 210272_at | 5.25E−04 | CYP2B7P1 |
| 7542 | 215281_x_at | 6.01E−04 | POGZ |
| 7542 | 243361_at | 3.00E−04 | SREK1 |
| 7550 | 1560576_at | 0.003 | — |
| 7550 | 214354_x_at | 0.001072 | SFTPB |
| 7564 | 1558887_at | 6.00E−05 | ZNF321P /// ZNF816 /// ZNF816-ZNF321P |
| 7565 | 1562579_at | 2.23E−05 | — |
| 7565 | 1566597_at | 1.36E−04 | — |
| 7565 | 233440_at | 1.72E−04 | — |
| 7565 | 205800_at | 8.00E−04 | — |
| 7565 | 1570432_at | 9.78E−04 | — |
| 7565 | 210334_x_at | 4.00E−08 | BIRC5 |
| 7565 | 223477_s_at | 2.99E−04 | C12orf65 |
| 7565 | 231859_at | 6.44E−05 | C14orf132 |
| 7565 | 243910_x_at | 1.42E−04 | CAND1 |
| 7565 | 222109_at | 2.50E−04 | GNL3L |
| 7565 | 220720_x_at | 3.53E−04 | MZT2B |
| 7565 | 1560145_at | 1.55E−05 | MKLN1 |
| 7565 | 207249_s_at | 2.28E−06 | SLC28A2 |
| 7565 | 1557275_a_at | 1.01E−04 | TLCD2 |
| 7565 | 1559289_at | 1.16E−04 | TADA1 |
| 7565 | 1569823_at | 0.001126 | LOC440346 |
| 7565 | 215625_at | 3.51E−06 | LOC644450 |
| 7565 | 208087_s_at | 4.00E−07 | ZBP1 |
| 7565 | 1552947_x_at | 1.92E−04 | ZNF114 |
| 7565 | 1552946_at | 1.92E−04 | ZNF114 |
| 7567 | 216155_at | 5.40E−04 | — |
| 7567 | 215597_x_at | 7.28E−04 | — |
| 7567 | 232991_at | 8.48E−04 | — |
| 7567 | 222207_x_at | 9.50E−04 | — |
| 7567 | 238829_at | 9.51E−04 | — |
| 7567 | 1569784_at | 0.001025 | — |
| 7567 | 1555617_x_at | 0.001124 | — |
| 7567 | 1555616_at | 0.001124 | — |
| 7567 | 233358_at | 0.001321 | — |
| 7567 | 215837_x_at | 0.001385 | — |
| 7567 | 242223_at | 0.003 | — |
| 7567 | 234148_at | 0.004 | — |
| 7567 | 221927_s_at | 7.66E−04 | ABHD11 |
| 7567 | 215135_at | 9.63E−04 | DNPEP |
| 7567 | 210858_x_at | 6.43E−04 | ATM |
| 7567 | 216875_x_at | 0.001183 | HAB1 |
| 7567 | 232885_at | 0.001251 | BBIP1 |
| 7567 | 206100_at | 0.004 | CPM |
| 7567 | 1559583_at | 0.001026 | CD276 |
| 7567 | 233693_at | 0.002075 | C1orf201 |
| 7567 | 242324_x_at | 7.92E−04 | CCBE1 |
| 7567 | 222104_x_at | 0.0022 | GTF2H3 |
| 7567 | 215202_at | 7.62E−04 | GUSBP11 |
| 7567 | 210224_at | 0.002001 | MR1 |
| 7567 | 215162_at | 0.001089 | RALGAPA1 |
| 7567 | 215208_x_at | 7.57E−04 | RPL35A |
| 7567 | 206995_x_at | 5.00E−04 | SCARF1 |
| 7567 | 222183_x_at | 0.001677 | SNW1 |
| 7567 | 204362_at | 0.004 | SKAP2 |
| 7567 | 203227_s_at | 0.00184 | TSPAN31 |
| 7567 | 203672_x_at | 0.00114 | TPMT |
| 7567 | 222267_at | 5.68E−04 | TMEM209 |
| 7567 | 224493_x_at | 0.001147 | TMEM241 |
| 7567 | 201671_x_at | 0.001556 | USP14 |
| 7567 | 1558527_at | 0.001055 | LOC100132707 |
| 7567 | 233992_x_at | 0.0014 | ZNF445 |
| 7567 | 1553022_at | 0.002007 | ZIM3 |
| 5865 | 1569761_x_at | 4.56E−04 | — |
| 5865 | 1569760_at | 4.56E−04 | — |
| 5865 | 240971_x_at | 6.90E−04 | — |
| 5865 | 1556814_a_at | 0.002333 | — |
| 5865 | 215803_at | 9.77E−04 | CXorf57 |
| 5865 | 215168_at | 0.001333 | TIMM17A |
| 5865 | 215978_x_at | 0.00184 | ZNF721 |
| 10016 | 211305_x_at | 0.001068 | FCAR |
| 10019 | 208162_s_at | 7.59E−04 | — |
| 10019 | 220809_at | 0.001 | — |
| 7602 | 234915_s_at | 0.004 | DENR |
| 7645 | 215995_x_at | 8.59E−04 | — |
| 7665 | 1561600_at | 2.00E−05 | — |
| 7665 | 220901_at | 7.00E−06 | GPR157 |
| 7665 | 215599_at | 1.00E−09 | GUSBP3 /// GUSBP9 |
| 7678 | 216733_s_at | 3.00E−14 | GATM |
| 7680 | 1558449_at | 2.00E−05 | — |
| 7680 | 212019_at | 8.00E−05 | RSL1D1 |
| 7680 | 219956_at | 2.53E−04 | GALNT6 |
| 7752 | 223679_at | 3.00E−04 | CTNNB1 |
| 7767 | 1562501_at | 3.00E−08 | LOC255177 |
| 7802 | 215385_at | 1.00E−05 | — |
| 7802 | 1562863_at | 3.00E−04 | — |
| 7802 | 243207_at | 0.0011 | — |
| 7802 | 222328_x_at | 0.001202 | — |
| 7802 | 233041_x_at | 0.001302 | — |
| 7802 | 232347_x_at | 0.001367 | — |
| 7802 | 234020_x_at | 0.00155 | — |
| 7802 | 1559229_at | 0.001852 | — |
| 7802 | 1565976_at | 0.002333 | — |
| 7802 | 207470_at | 0.003 | — |
| 7802 | 206924_at | 0.002 | IL11 |
| 7802 | 232457_at | 0.001483 | LIMCH1 |
| 7802 | 216466_at | 0.001 | NAV3 |
| 7802 | 1565786_x_at | 0.001077 | FLJ45482 |
| 7808 | 220387_s_at | 3.00E−04 | HHLA3 |
| 7808 | 234703_at | 3.00E−04 | HHLA3 |
| 7808 | 234665_x_at | 3.00E−04 | HHLA3 |
| 7816 | 1566129_at | 0.001054 | LIMS1 |
| 7839 | 235084_x_at | 4.00E−05 | TRIM38 |
| 7839 | 228353_x_at | 0.002 | UBASH3B |
| 7848 | 226913_s_at | 0.0015 | SOX8 |
| 7864 | 218661_at | 1.00E−09 | NAA60 |
| 7864 | 45526_g_at | 8.00E−06 | NAA60 |
| 7865 | 211412_at | 0.001039 | PADI4 |
| 7894 | 216132_at | 1.02E−04 | — |
| 7896 | 221501_x_at | 4.00E−10 | LOC100288332 /// LOC642778 /// LOC642799 /// PKD1P1 |
| 7897 | 241524_at | 2.00E−04 | — |
| 7897 | 1556778_at | 2.00E−04 | — |
| 7899 | 1560238_at | 0.001 | LOC100506930 |
| 7908 | 214035_x_at | 2.00E−10 | LOC399491 |
| 7908 | 204538_x_at | 2.00E−07 | NPIP |
| 7908 | 214870_x_at | 6.00E−04 | LOC100288332 /// LOC100506193 /// LOC642778 /// LOC642799 /// NPIP /// |
| 7916 | 240569_at | 5.45E−04 | — |
| 7933 | 234274_at | 4.30E−04 | — |
| 7936 | 1565788_at | 2.09E−04 | — |
| 7936 | 1554723_x_at | 0.002 | — |
| 7936 | 219384_s_at | 4.00E−05 | ADAT1 |
| 7936 | 1564307_a_at | 2.38E−04 | A2ML1 |
| 7936 | 206795_at | 8.00E−05 | F2RL2 |
| 7936 | 217535_at | 5.73E−04 | FAM49B |
| 7936 | 217534_at | 5.73E−04 | FAM49B |
| 7936 | 1569006_at | 2.21E−04 | LOC284379 |
| 7936 | 229296_at | 2.08E−04 | LOC100506119 |
| 7938 | 1559497_at | 8.00E−04 | — |
| 10025 | 239231_at | 8.00E−04 | — |
| 7964 | 240892_at | 8.00E−04 | — |
| 7964 | 234447_at | 0.003 | — |
| 7964 | 232753_at | 0.003 | ZNF346 |
| 7983 | 635_s_at | 0.003 | PPP2R5B |
| 7984 | 1566266_at | 2.00E−04 | — |
| 7984 | 1566265_at | 2.00E−04 | — |
| 7984 | 211369_at | 2.72E−04 | — |
| 7984 | 1557758_at | 3.40E−04 | — |
| 7984 | 216612_x_at | 4.01E−04 | — |
| 7984 | 219706_at | 1.75E−04 | AP5S1 |
| 7984 | 210873_x_at | 9.25E−05 | APOBEC3A |
| 7984 | 1555572_at | 4.69E−04 | CA6 |
| 7984 | 219621_at | 6.20E−04 | CLSPN |
| 7984 | 223255_at | 1.00E−04 | G2E3 |
| 7984 | 1570200_at | 2.60E−04 | HELB |
| 7984 | 216713_at | 5.00E−07 | KRIT1 |
| 7984 | 1559731_x_at | 6.58E−04 | MACROD1 |
| 7984 | 241395_at | 2.73E−04 | NIT1 |
| 7984 | 209422_at | 2.00E−04 | PHF20 |
| 7984 | 235218_x_at | 2.33E−04 | TIIAP6 |
| 7984 | 1569004_at | 1.54E−04 | LOC100505812 |
| 7991 | 1569551_at | 0.00178 | — |
| 7992 | 222272_x_at | 3.12E−04 | SCIN |
| 7994 | 223736_at | 0.002986 | IFT81 |
| 8002 | 233702_x_at | 4.82E−04 | — |
| 8002 | 234214_at | 8.88E−04 | — |
| 8002 | 211717_at | 0.003 | ANKRD40 |
| 8002 | 1562460_at | 0.00165 | CNDP2 |
| 8002 | 214372_x_at | 9.38E−04 | ERN2 |
| 8002 | 225164_s_at | 0.001733 | EIF2AK4 |
| 8002 | 222036_s_at | 0.003 | MCM4 |
| 8002 | 232158_x_at | 6.24E−04 | NIPAL1 |
| 8002 | 225335_at | 0.003 | ZNF496 |
| 8015 | 1562771_at | 1.69E−05 | — |
| 8015 | 215846_at | 2.51E−05 | — |
| 8015 | 239735_at | 2.51E−05 | — |
| 8015 | 221176_x_at | 3.84E−05 | — |
| 8015 | 216051_x_at | 5.26E−05 | — |
| 8015 | 240529_at | 1.00E−04 | — |
| 8015 | 1560129_at | 1.41E−04 | — |
| 8015 | 215507_x_at | 1.86E−04 | — |
| 8015 | 210679_x_at | 1.87E−04 | — |
| 8015 | 242080_at | 2.00E−04 | — |
| 8015 | 234046_at | 2.00E−04 | — |
| 8015 | 216745_x_at | 2.13E−04 | — |
| 8015 | 1570198_x_at | 2.75E−04 | — |
| 8015 | 234814_at | 2.75E−04 | — |
| 8015 | 216123_x_at | 2.97E−04 | — |
| 8015 | 1562947_x_at | 3.52E−04 | — |
| 8015 | 1562946_at | 3.52E−04 | — |
| 8015 | 215494_at | 3.75E−04 | — |
| 8015 | 1557325_at | 4.33E−04 | — |
| 8015 | 215609_at | 5.10E−04 | — |
| 8015 | 216789_at | 5.28E−04 | — |
| 8015 | 215314_at | 5.33E−04 | — |
| 8015 | 1557644_at | 5.37E−04 | — |
| 8015 | 235123_at | 5.38E−04 | — |
| 8015 | 222214_at | 5.50E−04 | — |
| 8015 | 233526_at | 5.51E−04 | — |
| 8015 | 1564336_at | 5.70E−04 | — |
| 8015 | 217679_x_at | 5.75E−04 | — |
| 8015 | 221071_at | 5.76E−04 | — |
| 8015 | 233721_x_at | 5.80E−04 | — |
| 8015 | 217706_at | 6.00E−04 | — |
| 8015 | 217164_at | 6.00E−04 | — |
| 8015 | 1559753_at | 6.00E−04 | — |
| 8015 | 1564148_at | 6.02E−04 | — |
| 8015 | 210920_x_at | 6.52E−04 | — |
| 8015 | 233218_at | 6.61E−04 | — |
| 8015 | 238672_at | 6.64E−04 | — |
| 8015 | 215742_at | 6.74E−04 | — |
| 8015 | 243103_at | 6.75E−04 | — |
| 8015 | 232962_x_at | 7.43E−04 | — |
| 8015 | 233519_at | 9.33E−04 | — |
| 8015 | 232714_at | 9.60E−04 | — |
| 8015 | 243660_at | 0.002 | — |
| 8015 | 231317_at | 0.0021 | — |
| 8015 | 207728_at | 7.33E−05 | ATF7IP |
| 8015 | 233333_x_at | 8.30E−04 | AVIL |
| 8015 | 1555435_at | 6.00E−04 | AFF4 |
| 8015 | 207133_x_at | 5.07E−04 | ALPK1 |
| 8015 | 209425_at | 0.002 | AMACR /// C1QTNF3 /// C1QTNF3-AMACR |
| 8015 | 223677_at | 4.50E−04 | ATG10 |
| 8015 | 1562391_at | 2.90E−04 | B3GALNT2 |
| 8015 | 221703_at | 6.32E−04 | BRIP1 |
| 8015 | 209508_x_at | 2.51E−05 | CFLAR |
| 8015 | 211316_x_at | 3.50E−04 | CFLAR |
| 8015 | 209790_s_at | 0.00125 | CASP6 |
| 8015 | 201974_s_at | 6.00E−04 | CCZ1 |
| 8015 | 205264_at | 4.45E−04 | CD3EAP |
| 8015 | 244352_at | 1.84E−05 | CD84 |
| 8015 | 1558557_at | 6.00E−04 | C16orf62 |
| 8015 | 1564315_at | 4.78E−04 | C8orf49 |
| 8015 | 1559003_a_at | 6.00E−04 | CCDC163P |
| 8015 | 211325_x_at | 0.001066 | DSTNP2 |
| 8015 | 1555404_a_at | 0.002 | DUOXA1 |
| 8015 | 215173_at | 8.61E−04 | DNAAF1 |
| 8015 | 220725_x_at | 0.001407 | DNAH3 |
| 8015 | 1559044_at | 2.10E−04 | EXOSC1 |
| 8015 | 226062_x_at | 4.66E−04 | FAM63A |
| 8015 | 232350_x_at | 2.67E−04 | GPR161 |
| 8015 | 234918_at | 9.14E−05 | GLTSCR2 |
| 8015 | 206952_at | 1.93E−04 | G6PC |
| 8015 | 223327_x_at | 3.03E−04 | AGSK1 /// LOC727751 /// LOC727849 |
| 8015 | 1557350_at | 0.002 | G3BP1 |
| 8015 | 1552318_at | 7.37E−04 | GIMAP1 |
| 8015 | 232043_at | 2.00E−04 | GNG7 |
| 8015 | 220071_x_at | 5.57E−04 | HAUS2 |
| 8015 | 236400_at | 2.00E−04 | IDH1-AS1 |
| 8015 | 1569033_at | 5.14E−04 | IGF2BP3 |
| 8015 | 1560172_at | 8.28E−04 | INTS10 |
| 8015 | 207194_s_at | 0.0021 | ICAM4 |
| 8015 | 1552584_at | 0.0015 | IL12RB1 |
| 8015 | 209245_s_at | 5.61E−04 | KIF1C |
| 8015 | 244276_at | 5.02E−05 | KLB |
| 8015 | 215322_at | 1.70E−05 | LONRF1 |
| 8015 | 210711_at | 0.00181 | LINC00260 |
| 8015 | 231840_x_at | 2.52E−05 | LYRM7 |
| 8015 | 202793_at | 7.42E−04 | LPCAT3 |
| 8015 | 208306_x_at | 1.09E−04 | HLA-DRB1 /// LOC100507709 /// LOC100507714 |
| 8015 | 221697_at | 4.63E−05 | MAP1LC3C |
| 8015 | 1569348_at | 8.72E−04 | MIR4500HG /// TPTEP1 |
| 8015 | 216206_x_at | 1.69E−05 | MAP2K7 |
| 8015 | 217295_at | 1.68E−05 | MUC8 |
| 8015 | 211445_x_at | 2.91E−04 | NACAP1 |
| 8015 | 1557737_s_at | 1.67E−05 | NKTR |
| 8015 | 207631_at | 1.03E−04 | NBR2 |
| 8015 | 214123_s_at | 1.00E−04 | NOP14-AS1 |
| 8015 | 222255_at | 0.002035 | PRX |
| 8015 | 244689_at | 9.34E−04 | PPARA |
| 8015 | 206792_x_at | 5.30E−04 | PDE4C |
| 8015 | 210304_at | 3.92E−04 | PDE6B |
| 8015 | 1552343_s_at | 1.70E−04 | PDE7A |
| 8015 | 231812_x_at | 1.33E−04 | PHAX |
| 8015 | 220714_at | 6.67E−05 | PRDM14 |
| 8015 | 222106_at | 3.38E−04 | PRND |
| 8015 | 207291_at | 2.70E−05 | PRRG4 |
| 8015 | 1569748_at | 0.001411 | PRKCZ |
| 8015 | 231830_x_at | 7.34E−04 | RAB11FIP1 |
| 8015 | 212706_at | 0.002063 | RASA4 /// RASA4B |
| 8015 | 1563014_at | 6.00E−04 | RPS15 |
| 8015 | 1557981_at | 1.00E−04 | RPS9 |
| 8015 | 207735_at | 1.45E−04 | RNF125 |
| 8015 | 207889_at | 0.002 | SARDH |
| 8015 | 232421_at | 1.00E−04 | SCARB1 |
| 8015 | 222169_x_at | 5.60E−04 | SH2D3A |
| 8015 | 1552910_at | 4.00E−04 | SIGLEC11 |
| 8015 | 229209_at | 0.001052 | SNHG10 |
| 8015 | 1568922_at | 4.00E−04 | SLC24A4 |
| 8015 | 1560062_at | 0.001067 | SLC29A2 |
| 8015 | 205482_x_at | 7.63E−04 | SNX15 |
| 8015 | 214791_at | 1.26E−05 | SP140L |
| 8015 | 1555614_at | 6.00E−04 | SUGT1P1 |
| 8015 | 1569073_x_at | 2.70E−04 | SMARCA4 |
| 8015 | 215122_at | 8.02E−04 | TBX6 |
| 8015 | 1566901_at | 0.001067 | TGIF1 |
| 8015 | 1562490_at | 7.53E−04 | THEM5 |
| 8015 | 1569392_at | 6.33E−06 | TECR |
| 8015 | 226483_at | 7.92E−04 | TMEM68 |
| 8015 | 215844_at | 1.69E−05 | TNPO2 |
| 8015 | 1554182_at | 1.10E−04 | TRIM73 /// TRIM74 |
| 8015 | 216439_at | 4.14E−04 | TNK2 |
| 8015 | 211008_s_at | 0.001293 | UBE2I |
| 8015 | 1552834_at | 2.03E−04 | B3GNT6 |
| 8015 | 214809_at | 3.33E−04 | LOC100294145 |
| 8015 | 232804_at | 2.63E−04 | LOC100506385 |
| 8015 | 1564232_at | 5.48E−04 | LOC100506453 /// LOC441528 |
| 8015 | 235014_at | 3.50E−05 | LOC147727 |
| 8015 | 1563571_at | 3.27E−04 | LOC285463 |
| 8015 | 220352_x_at | 9.71E−04 | FLJ42627 |
| 8016 | 215840_at | 5.01E−04 | DNAH2 |
| 8016 | 217497_at | 5.50E−04 | TYMP |
| 8016 | 235317_at | 1.00E−05 | LOC284454 |
| 8016 | 1555847_a_at | 1.00E−05 | LOC284454 |
| 8017 | 234953_x_at | 3.00E−13 | ZNF19 |
| 8034 | 208319_s_at | 6.00E−04 | RBM3 |
| 8057 | 1565868_at | 0.005 | CD44 |
| 8097 | 238360_s_at | 0.003 | LOC100505576 |
| 8105 | 217033_x_at | 0.003 | NTRK3 |
| 8142 | 216118_at | 0.002 | — |
| 8147 | 220578_at | 0.002 | ADAMTSL4 |
| 8147 | 206829_x_at | 0.001 | ZNF430 |
| 8156 | 216290_x_at | 3.25E−04 | — |
| 8156 | 1569952_x_at | 3.61E−04 | — |
| 8156 | 242740_at | 3.96E−04 | — |
| 8156 | 1563505_at | 4.12E−04 | DUSP16 |
| 8156 | 212394_at | 0.001401 | EMC1 |
| 8156 | 208247_at | 0.001433 | ERC2-IT1 |
| 8156 | 217618_x_at | 4.33E−04 | HUS1 |
| 8156 | 207433_at | 8.03E−04 | IL10 |
| 8156 | 208262_x_at | 9.11E−04 | MEFV |
| 8156 | 215123_at | 0.001758 | 61E3.4 /// LOC100132247 /// LOC100652992 /// LOC613037 /// LOC728888 /// |
| 8156 | 1570210_x_at | 8.20E−04 | PPP6R2 |
| 8156 | 203971_at | 4.18E−04 | SLC31A1 |
| 8156 | 206293_at | 7.96E−04 | SULT2A1 |
| 8156 | 203568_s_at | 0.00101 | TRIM38 |
| 8156 | 206916_x_at | 5.12E−04 | TAT |
| 8156 | 214413_at | 5.63E−04 | TAT |
| 8156 | 207499_x_at | 3.14E−04 | UNC45A |
| 8156 | 1564639_at | 0.00225 | LOC100291323 |
| 8165 | 240620_at | 7.00E−04 | — |
| 8165 | 223290_at | 7.00E−04 | PDXP /// SH3BP1 |
| 5925 | 1566202_at | 2.00E−05 | — |
| 5925 | 220696_at | 7.00E−04 | — |
| 5925 | 233776_at | 0.003 | — |
| 5925 | 1569728_at | 0.003 | LINC00423 |
| 5925 | 201695_s_at | 7.00E−04 | PNP |
| 5925 | 231961_at | 0.00185 | RBPMS |
| 5925 | 242358_at | 7.00E−04 | LOC100506451 |
| 8179 | 217098_s_at | 0.003 | ZSCAN12 |
| 8179 | 1558622_a_at | 2.00E−04 | ZNF548 |
| 8179 | 1568900_a_at | 0.003 | ZNF568 |
| 8179 | 217627_at | 0.003 | ZNF573 |
| 8179 | 1559789_a_at | 8.00E−04 | ZNF781 |
| 5927 | 1561879_at | 2.50E−06 | — |
| 5927 | 1560678_at | 5.31E−06 | — |
| 5927 | 215907_at | 1.47E−05 | — |
| 5927 | 1569478_s_at | 4.40E−05 | — |
| 5927 | 1561704_at | 6.40E−05 | — |
| 5927 | 236839_at | 8.28E−05 | — |
| 5927 | 232363_at | 1.18E−04 | — |
| 5927 | 233004_x_at | 1.55E−04 | — |
| 5927 | 1555718_x_at | 1.91E−04 | — |
| 5927 | 1555717_at | 1.91E−04 | — |
| 5927 | 215553_x_at | 2.13E−04 | — |
| 5927 | 1561924_at | 2.50E−04 | — |
| 5927 | 1566887_x_at | 2.53E−04 | — |
| 5927 | 216187_x_at | 3.45E−04 | — |
| 5927 | 231212_x_at | 4.69E−04 | — |
| 5927 | 241094_at | 6.59E−04 | — |
| 5927 | 215373_x_at | 7.29E−04 | — |
| 5927 | 207436_x_at | 8.08E−04 | — |
| 5927 | 222359_x_at | 1.43E−04 | KDSR |
| 5927 | 241354_at | 5.04E−05 | ASMTL-AS1 |
| 5927 | 207288_at | 4.25E−04 | ASMTL-AS1 |
| 5927 | 204920_at | 0.0019 | CPS1 |
| 5927 | 225836_s_at | 4.46E−04 | C12orf32 |
| 5927 | 1554176_a_at | 2.29E−04 | C3orf33 |
| 5927 | 1555095_at | 3.37E−04 | C6orf123 |
| 5927 | 1569293_x_at | 2.65E−04 | CROCCP2 |
| 5927 | 1569291_at | 2.65E−04 | CROCCP2 |
| 5927 | 220774_at | 1.00E−06 | DYM |
| 5927 | 222194_at | 3.00E−04 | FAM66D |
| 5927 | 219815_at | 1.00E−06 | GAL3ST4 |
| 5927 | 233193_x_at | 1.17E−04 | INTS4 |
| 5927 | 1553243_at | 0.001302 | ITIH5 |
| 5927 | 216958_s_at | 4.00E−06 | IVD |
| 5927 | 230567_at | 1.01E−04 | KIAA1430 |
| 5927 | 211452_x_at | 2.80E−04 | LRRFIP1 |
| 5927 | 217810_x_at | 2.13E−04 | LARS |
| 5927 | 222762_x_at | 5.02E−04 | LIMD1 |
| 5927 | 220945_x_at | 4.76E−04 | MANSC1 |
| 5927 | 234976_x_at | 1.00E−06 | MTHFD2 |
| 5927 | 214650_x_at | 5.76E−04 | MOG |
| 5927 | 229220_x_at | 6.67E−04 | NOM1 |
| 5927 | 215179_x_at | 1.46E−04 | PGF |
| 5927 | 207109_at | 7.23E−04 | POU2F3 |
| 5927 | 1554376_s_at | 0.002525 | PTPLA |
| 5927 | 223927_at | 2.66E−04 | PCDHB9 |
| 5927 | 1565800_x_at | 3.35E−04 | RAB3IP |
| 5927 | 1565799_at | 3.35E−04 | RAB3IP |
| 5927 | 1569396_at | 2.13E−04 | RAB40C |
| 5927 | 233872_x_at | 4.78E−05 | ARHGAP5 |
| 5927 | 1564377_at | 3.68E−07 | SHANK2 |
| 5927 | 1552497_a_at | 7.67E−04 | SLAMF6 |
| 5927 | 220182_at | 1.00E−06 | SLC25A23 |
| 5927 | 220181_x_at | 6.14E−04 | SLC30A5 |
| 5927 | 243594_x_at | 0.001 | SPIRE2 |
| 5927 | 222907_x_at | 5.66E−04 | TMEM50B |
| 5927 | 1566152_a_at | 5.60E−05 | LOC100507106 |
| 5927 | 1566734_at | 1.77E−04 | LOC283454 |
| 5927 | 1558723_at | 2.84E−04 | LOC284014 |
| 5927 | 232455_x_at | 0.001002 | LOC340085 |
| 5927 | 1561099_at | 2.91E−05 | FLJ32756 |
| 5927 | 224185_at | 3.41E−04 | WRAP53 |
| 5927 | 205519_at | 8.98E−04 | WDR76 |
| 5927 | 239243_at | 1.00E−06 | ZNF638 |
| 5927 | 1561038_at | 5.20E−07 | ZNF81 |
| 8195 | 1566098_at | 4.47E−04 | — |
| 8195 | 1554839_at | 6.69E−05 | CIDECP |
| 8195 | 231994_at | 9.01E−07 | CHDH |
| 8195 | 1559591_s_at | 1.98E−05 | CHDH |
| 8195 | 207516_at | 4.00E−05 | CHRNB4 |
| 8195 | 216459_x_at | 9.00E−04 | DDR1-AS1 |
| 8195 | 242945_at | 5.00E−07 | FAM20A |
| 8195 | 210429_at | 5.00E−04 | RHD |
| 8195 | 233360_at | 0.001268 | UBE2I |
| 8195 | 1564021_at | 8.18E−04 | LOC100130456 |
| 8195 | 1569078_at | 1.05E−07 | LOC100294362 |
| 8198 | 1563303_at | 4.80E−05 | — |
| 8198 | 1561139_at | 2.67E−04 | — |
| 8198 | 215163_at | 4.23E−04 | — |
| 8198 | 243037_at | 0.001333 | — |
| 8198 | 1558577_at | 1.67E−04 | LOC148709 |
| 8198 | 219857_at | 0.0011 | PLEKHS1 |
| 8198 | 215359_x_at | 7.00E−06 | ZNF44 |
| 10041 | 1557897_at | 3.00E−04 | — |
| 10041 | 1562384_at | 4.00E−04 | — |
| 10041 | 241848_x_at | 6.55E−04 | — |
| 10041 | 1570491_at | 0.0011 | — |
| 10041 | 1569213_at | 7.45E−04 | LOC400891 |
| 10041 | 234098_at | 0.001375 | SOBP |
| 8240 | 234880_x_at | 4.00E−04 | KRTAP1-3 |
| 8246 | 223647_x_at | 0.0021 | HSCB |
| 8248 | 1557864_x_at | 3.04E−04 | — |
| 8251 | 234517_at | 2.78E−04 | — |
| 8251 | 1557135_at | 3.70E−04 | — |
| 8251 | 1566696_at | 4.39E−04 | — |
| 8251 | 208246_x_at | 4.65E−04 | — |
| 8251 | 1570295_at | 5.24E−04 | — |
| 8251 | 1561741_at | 7.10E−04 | — |
| 8251 | 244711_at | 0.001505 | — |
| 8251 | 226544_x_at | 5.89E−04 | EEF1E1-MUTED /// MUTED |
| 8251 | 1556436_at | 3.03E−04 | EXTL3-AS1 |
| 8251 | 216502_at | 5.70E−04 | ISG20L2 |
| 8251 | 202903_at | 0.0015 | LSM5 |
| 8251 | 206426_at | 0.001159 | MLANA |
| 8251 | 217677_at | 0.00144 | PLEKHA2 |
| 8251 | 208411_x_at | 2.32E−04 | PPEF2 |
| 8251 | 1569502_s_at | 8.46E−04 | TP73-AS1 |
| 8251 | 206169_x_at | 3.52E−04 | ZC3H7B |
| 8251 | 1558586_at | 5.72E−04 | ZNF33B |
| 8251 | 1553901_x_at | 0.001428 | ZNF486 |
| 8251 | 1552770_s_at | 3.04E−04 | ZNF563 |
| 8269 | 234501_x_at | 1.33E−04 | — |
| 8269 | 233783_at | 1.76E−04 | — |
| 8269 | 242658_at | 4.00E−04 | — |
| 8303 | 236438_at | 0.001 | — |
| 8303 | 208014_x_at | 0.001445 | — |
| 8303 | 1563080_at | 0.002001 | — |
| 8303 | 1566703_at | 0.0022 | — |
| 8303 | 244665_at | 0.0025 | — |
| 8303 | 213026_at | 0.002004 | ATG12 |
| 8303 | 240366_at | 0.001 | LHFPL3-AS1 |
| 8303 | 210556_at | 6.32E−04 | NFATC3 |
| 8303 | 1558738_at | 0.001401 | NOL3 |
| 8306 | 1557486_at | 3.52E−06 | — |
| 8306 | 243931_at | 4.50E−06 | — |
| 8306 | 208238_x_at | 1.10E−05 | — |
| 8306 | 233037_at | 5.00E−05 | — |
| 8306 | 210598_at | 1.03E−04 | — |
| 8306 | 1569712_at | 1.69E−04 | — |
| 8306 | 242731_x_at | 1.95E−04 | — |
| 8306 | 232626_at | 2.36E−04 | — |
| 8306 | 1559652_at | 2.59E−04 | — |
| 8306 | 1559663_at | 3.00E−04 | — |
| 8306 | 1563461_at | 5.22E−04 | — |
| 8306 | 243391_x_at | 5.40E−04 | — |
| 8306 | 1560792_at | 5.52E−04 | — |
| 8306 | 236153_at | 6.32E−04 | — |
| 8306 | 216771_at | 6.50E−04 | — |
| 8306 | 242413_at | 6.70E−04 | — |
| 8306 | 207730_x_at | 7.56E−04 | — |
| 8306 | 1570496_at | 0.00104 | — |
| 8306 | 205745_x_at | 5.00E−07 | ADAM17 |
| 8306 | 231858_x_at | 5.04E−04 | AP5B1 |
| 8306 | 232814_x_at | 0.00154 | APOPT1 |
| 8306 | 1554367_at | 2.00E−05 | CALHM1 |
| 8306 | 223942_x_at | 7.00E−06 | CHST5 |
| 8306 | 243403_x_at | 2.00E−05 | CPM |
| 8306 | 232650_at | 2.00E−05 | C12orf65 |
| 8306 | 220601_at | 5.81E−04 | C16orf70 |
| 8306 | 219678_x_at | 1.36E−04 | DCLRE1C |
| 8306 | 208963_x_at | 0.00154 | FADS1 /// MIR1908 |
| 8306 | 210608_s_at | 8.00E−05 | FUT2 |
| 8306 | 1556742_at | 2.00E−05 | GUSBP1 |
| 8306 | 211351_at | 8.00E−05 | KIF25-AS1 |
| 8306 | 206500_s_at | 0.001693 | MIS18BP1 |
| 8306 | 220411_x_at | 4.35E−05 | PODNL1 |
| 8306 | 211265_at | 6.93E−04 | PTGER3 |
| 8306 | 1560014_s_at | 3.52E−06 | PDXDC1 |
| 8306 | 1560013_at | 3.01E−04 | PDXDC1 |
| 8306 | 227888_at | 3.00E−04 | RRBP1 |
| 8306 | 238087_at | 0.001218 | RTCA |
| 8306 | 229334_at | 0.00104 | RUFY3 |
| 8306 | 1569938_at | 5.10E−04 | SIRT5 |
| 8306 | 1564911_at | 2.11E−04 | SNHG4 |
| 8306 | 232355_at | 0.001001 | SNORD114-3 |
| 8306 | 211855_s_at | 2.00E−05 | SLC25A14 |
| 8306 | 1557073_s_at | 0.00101 | TTBK2 |
| 8306 | 217625_x_at | 3.50E−04 | LOC100506190 |
| 8306 | 235617_x_at | 8.00E−06 | LOC100507537 |
| 8306 | 233013_x_at | 5.04E−04 | LOC220906 |
| 8306 | 1566760_at | 7.00E−06 | FLJ34208 |
| 8306 | 1554476_x_at | 7.00E−06 | ZNF808 |
| 8311 | 242826_at | 0.001 | — |
| 8311 | 1561276_at | 3.81E−04 | DOCK5 |
| 8318 | 1566106_at | 0.002 | — |
| 8318 | 230309_at | 3.36E−06 | BHMT2 |
| 8318 | 213275_x_at | 1.00E−05 | CTSB |
| 8318 | 211042_x_at | 0.002 | MCAM |
| 8318 | 219788_at | 0.002 | PILRA |
| 8318 | 218997_at | 3.10E−04 | POLR1E |
| 8318 | 1559169_at | 0.002 | LOC100507013 |
| 8319 | 215674_at | 1.50E−04 | — |
| 8319 | 1560563_at | 2.52E−04 | — |
| 8319 | 1564472_at | 5.45E−04 | — |
| 8319 | 234478_at | 5.45E−04 | — |
| 8319 | 216729_at | 0.001 | — |
| 8319 | 233622_x_at | 0.001 | — |
| 8319 | 1561918_at | 0.001 | — |
| 8319 | 239053_at | 6.46E−04 | CIAO1 |
| 8319 | 233741_at | 5.33E−04 | FTCD |
| 8319 | 1569503_at | 5.52E−04 | HEATR5B |
| 8319 | 232627_at | 3.39E−04 | HGS |
| 8319 | 1569980_x_at | 5.00E−04 | HKR1 |
| 8319 | 1569979_at | 5.00E−04 | HKR1 |
| 8319 | 1553484_at | 0.0025 | LINC00477 |
| 8319 | 221192_x_at | 0.004 | MFSD11 |
| 8319 | 215895_x_at | 0.001 | PLIN2 |
| 8319 | 207836_s_at | 9.00E−05 | RBPMS |
| 8319 | 207837_at | 9.00E−05 | RBPMS |
| 8319 | 1566191_at | 0.001 | SUZ12 |
| 8319 | 207554_x_at | 3.33E−04 | TBXA2R |
| 8319 | 220655_at | 1.45E−04 | TNIP3 |
| 8319 | 1552648_a_at | 9.50E−04 | TNFRSF10A |
| 8319 | 1562051_at | 0.001 | LIC284757 /// LOC729296 |
| 8324 | 240148_at | 1.00E−04 | — |
| 8324 | 240800_x_at | 5.00E−04 | — |
| 8324 | 1569738_at | 5.00E−04 | — |
| 8324 | 221184_at | 5.80E−04 | — |
| 8324 | 207953_at | 0.001001 | — |
| 8324 | 1556815_at | 0.0011 | — |
| 8324 | 241443_at | 0.001112 | — |
| 8324 | 233258_at | 0.00135 | — |
| 8324 | 204573_at | 0.002 | CROT |
| 8324 | 220575_at | 3.50E−04 | FAM106A |
| 8324 | 1553361_x_at | 0.002 | FBXL18 |
| 8324 | 1553359_at | 0.002 | FBXL18 |
| 8324 | 203607_at | 5.13E−04 | INPP5F |
| 8324 | 1558425_x_at | 0.002 | LINC00265 /// LOC100292676 /// LOC730376 |
| 8324 | 1564658_at | 2.90E−04 | NAT16 |
| 8324 | 239362_at | 5.00E−04 | NAPA-AS1 |
| 8324 | 205296_at | 8.26E−04 | RBL1 |
| 8324 | 202335_s_at | 7.50E−04 | UBE2B |
| 8324 | 1558148_x_at | 0.003 | FLJ90757 |
| 8330 | 1570105_at | 2.00E−04 | — |
| 17 | 230694_at | 1.00E−08 | SLC35E2B |
| 8366 | 1560347_at | 6.12E−04 | — |
| 8366 | 1566870_at | 9.33E−04 | — |
| 8366 | 233291_at | 0.00135 | — |
| 8366 | 231856_at | 0.002 | KIAA1244 |
| 8366 | 206451_at | 7.16E−04 | TBCCD1 |
| 19 | 1556951_at | 4.00E−04 | — |
| 19 | 235006_at | 7.00E−07 | CDKN2AIPNL |
| 19 | 221170_at | 0.005 | HRH4 |
| 19 | 201013_s_at | 0.005 | PAICS |
| 8378 | 203277_at | 4.00E−04 | DFFA |
| 8384 | 1558021_at | 4.00E−08 | RABEPK |
| 8416 | 213553_x_at | 0.003 | APOC1 |
| 8419 | 235999_at | 0.002 | — |
| 8419 | 206809_s_at | 1.00E−05 | HNRNPA3 /// HNRNPA3P1 |
| 8419 | 206808_at | 1.00E−05 | HNRNPA3P1 |
| 8419 | 218047_at | 0.002 | OSBPL9 |
| 8419 | 203544_s_at | 1.00E−05 | STAM |
| 8419 | 227233_at | 0.002 | TSPAN2 |
| 8419 | 238958_at | 0.002 | UBE2E1 |
| 8442 | 216691_at | 5.00E−06 | — |
| 8442 | 238738_at | 5.00E−06 | — |
| 8452 | 1559147_at | 2.00E−04 | — |
| 8462 | 1565604_at | 3.00E−04 | PWP2 |
| 8462 | 1554676_at | 4.63E−04 | SRGN |
| 8462 | 220826_at | 4.00E−04 | TCP10L |
| 8495 | 217650_x_at | 3.10E−04 | ST3GAL2 |
| 8534 | 1563638_at | 7.56E−04 | FAM18A |
| 8587 | 1561151_a_at | 8.00E−04 | — |
| 8594 | 243667_at | 1.00E−04 | — |
| 8594 | 233875_at | 3.50E−04 | — |
| 8594 | 215504_x_at | 3.50E−04 | — |
| 8594 | 212172_at | 8.70E−05 | AK2 |
| 8594 | 1559097_at | 5.50E−05 | C14orf64 |
| 8594 | 233122_at | 3.00E−05 | KRTCAP2 |
| 8594 | 219730_at | 6.71E−05 | MED18 |
| 8594 | 232648_at | 1.02E−04 | PSMA3 |
| 8594 | 242208_at | 3.00E−07 | ZNF37BP |
| 45 | 1554550_at | 0.001504 | KIAA1430 |
| 45 | 232579_at | 0.003 | LOC100134229 |
| 45 | 1556319_at | 0.003 | LOC283270 |
| 8664 | 207417_s_at | 5.00E−11 | ZNF177 /// ZNF559-ZNF177 |
| 8664 | 223714_at | 6.00E−07 | ZNF256 |
| 8664 | 235414_at | 3.00E−05 | ZNF383 |
| 8664 | 208081_s_at | 0.004 | ZNF442 |
| 8664 | 215315_at | 6.00E−07 | ZNF549 |
| 8678 | 229033_s_at | 4.00E−16 | MUM1 |
| 8680 | 242645_at | 3.50E−06 | — |
| 8680 | 1562962_at | 7.00E−06 | — |
| 8680 | 1570103_at | 3.00E−04 | — |
| 8680 | 1570205_at | 0.001 | — |
| 8680 | 244193_at | 0.003 | DNAJC22 |
| 8680 | 220974_x_at | 2.00E−05 | SFXN3 |
| 8686 | 233056_x_at | 0.00103 | DLGAP4 |
| 8687 | 215901_at | 0.0021 | ZNF81 |
| 8699 | 217708_x_at | 4.00E−05 | — |
| 8699 | 1560370_x_at | 1.01E−05 | ANKH |
| 8699 | 1560369_at | 1.01E−05 | ANKH |
| 8699 | 238723_at | 3.45E−05 | ATXN3 |
| 8699 | 219149_x_at | 0.00144 | DBR1 |
| 8699 | 222842_at | 0.002404 | EIF2C4 |
| 8699 | 1552664_at | 0.001505 | FLCN |
| 8699 | 233605_x_at | 5.53E−04 | IINRNPM |
| 8699 | 212594_at | 5.42E−06 | MIR4680 /// PDCD4 |
| 8699 | 1559127_x_at | 7.30E−04 | RRP12 |
| 8699 | 219125_s_at | 7.79E−04 | SLC50A1 |
| 8699 | 215383_x_at | 0.001513 | SPG21 |
| 8699 | 1569723_a_at | 2.00E−05 | SPIRE2 |
| 8699 | 204341_at | 5.05E−04 | TRIM16 |
| 8699 | 223109_at | 3.00E−04 | TRUB2 |
| 8699 | 1569378_at | 5.00E−05 | LOC100292680 |
| 8699 | 215467_x_at | 0.001605 | LOC647070 |
| 8699 | 204590_x_at | 1.65E−04 | VPS33A |
| 8699 | 214715_x_at | 8.76E−04 | ZNF160 |
| 8699 | 219765_at | 2.04E−04 | ZNF329 |
| 8699 | 206188_at | 1.90E−04 | ZNF623 |
| 8708 | 220444_at | 1.00E−13 | ZNF557 |
| 8763 | 1566503_at | 1.70E−04 | — |
| 8766 | 234193_at | 4.92E−04 | — |
| 8768 | 227234_at | 3.00E−10 | LOC100132815 |
| 8792 | 215196_at | 2.00E−05 | — |
| 8792 | 233677_at | 1.10E−04 | — |
| 8792 | 234190_at | 2.58E−04 | — |
| 8792 | 232144_at | 3.20E−04 | — |
| 8792 | 1552509_a_at | 2.00E−05 | CD300LG |
| 8792 | 225708_at | 4.00E−05 | MED29 |
| 8792 | 1564093_at | 2.00E−04 | NEK1 |
| 8792 | 219347_at | 6.49E−04 | NUDT15 |
| 8792 | 241972_at | 4.00E−05 | LOC401588 |
| 8799 | 208368_s_at | 6.04E−04 | BRCA2 |
| 8799 | 215374_at | 1.00E−10 | PAPOLA |
| 8799 | 235448_at | 6.00E−08 | ZXDC |
| 8806 | 1562731_s_at | 4.00E−05 | MDS2 |
| 8812 | 220019_s_at | 3.50E−07 | ZNF224 |
| 8821 | 1561408_at | 0.005 | — |
| 8821 | 240068_at | 0.005 | LINC00323 |
| 8821 | 207084_at | 0.005 | POU3F2 |
| 8824 | 206203_at | 0.001 | RCVRN |
| 8829 | 1569830_at | 0.001 | PTPRC |
| 8835 | 242978_x_at | 0.00195 | — |
| 8837 | 215919_s_at | 0.005 | MRPS11 |
| 8837 | 210580_x_at | 1.00E−04 | SLX1A-SULT1A3 /// SLX1B-SULT1A4 /// SULT1A3 /// SULT1A4 |
| 8837 | 243553_x_at | 0.005 | TRAF3IP2-AS1 |
| 8837 | 217220_at | 0.005 | LOC100287387 |
| 8837 | 1559059_s_at | 0.001367 | ZNF611 |
| 8837 | 217541_x_at | 6.50E−05 | ZNF816 /// ZNF816-ZNF321P |
| 8841 | 237768_x_at | 1.65E−04 | — |
| 8851 | 233754_x_at | 4.00E−04 | ZNF71 |
| 63 | 1565621_at | 3.00E−05 | — |
| 63 | 1552674_at | 5.00E−09 | DIRC1 |
| 63 | 221536_s_at | 2.00E−07 | LSG1 |
| 63 | 215302_at | 2.50E−09 | LOC257152 |
| 8867 | 233616_at | 7.50E−04 | — |
| 8867 | 237451_x_at | 0.001 | — |
| 8867 | 232511_at | 0.001 | — |
| 8867 | 239235_at | 0.00253 | — |
| 8867 | 221980_at | 3.33E−04 | EMILIN2 |
| 8867 | 205085_at | 0.001 | ORC1 |
| 8867 | 224226_at | 0.005 | PKD2L2 |
| 8867 | 230490_x_at | 0.001 | RSU1 |
| 8867 | 206529_x_at | 3.64E−04 | SLC26A4 |
| 8867 | 235325_at | 0.005 | SPG7 |
| 8867 | 232588_at | 4.00E−04 | STAG1 |
| 8867 | 1562470_at | 0.001 | TCEANC2 |
| 8867 | 235587_at | 0.001 | LOC202781 |
| 8889 | 244677_at | 1.67E−04 | — |
| 8889 | 1569772_x_at | 2.05E−04 | — |
| 8889 | 214605_x_at | 1.25E−04 | GPR1 |
| 8889 | 1560448_at | 1.68E−04 | PRR7-AS1 |
| 66 | 1568755_a_at | 2.00E−04 | — |
| 66 | 237489_at | 7.00E−04 | — |
| 66 | 243442_x_at | 7.00E−04 | — |
| 66 | 243866_x_at | 7.00E−04 | — |
| 66 | 224119_at | 7.00E−04 | — |
| 66 | 1561606_at | 7.00E−04 | — |
| 66 | 233824_at | 0.0016 | — |
| 66 | 231825_x_at | 0.002 | ATF7IP |
| 66 | 1565939_at | 7.00E−04 | C5orf22 |
| 66 | 241784_x_at | 0.002 | HELQ |
| 66 | 235783_at | 2.00E−04 | MRTO4 |
| 66 | 217526_at | 7.00E−04 | NFATC2IP |
| 66 | 216755_at | 7.00E−04 | OSBPL10 |
| 66 | 206056_x_at | 2.00E−04 | SPN |
| 66 | 1553020_at | 0.002 | SMCR5 |
| 66 | 204124_at | 6.00E−05 | SLC34A2 |
| 66 | 1570293_at | 7.00E−04 | TBL1X |
| 66 | 215892_at | 2.00E−04 | ZNF440 |
| 67 | 215019_x_at | 9.01E−04 | ZNF528 |
| 8915 | 232338_at | 3.56E−04 | — |
| 5931 | 221653_x_at | 5.00E−06 | APOL2 |
| 8960 | 243658_at | 3.50E−06 | — |
| 8960 | 237561_x_at | 0.00105 | — |
| 8960 | 220255_at | 1.00E−04 | FANCE |
| 8960 | 243630_at | 3.00E−05 | NDUFB1 |
| 5935 | 236802_at | 3.00E−04 | — |
| 8991 | 242423_x_at | 1.00E−04 | — |
| 8991 | 236801_at | 0.001 | — |
| 8991 | 1570331_at | 0.0011 | — |
| 8991 | 1566123_at | 5.01E−05 | FABP6 |
| 8991 | 210705_s_at | 5.45E−05 | TRIM5 |
| 5937 | 215636_at | 6.73E−05 | UBR4 |
| 8998 | 242950_x_at | 1.34E−05 | — |
| 8998 | 237245_at | 3.01E−05 | — |
| 8998 | 221130_s_at | 1.00E−04 | — |
| 8998 | 1563052_at | 1.01E−04 | — |
| 8998 | 1560026_at | 2.00E−04 | — |
| 8998 | 1565596_at | 3.12E−04 | — |
| 8998 | 1570645_at | 4.02E−04 | — |
| 8998 | 234765_at | 4.50E−04 | — |
| 8998 | 232743_at | 7.00E−04 | — |
| 8998 | 1557908_at | 7.00E−04 | — |
| 8998 | 1555117_at | 7.00E−04 | — |
| 8998 | 234113_at | 8.34E−04 | — |
| 8998 | 232867_at | 8.50E−04 | — |
| 8998 | 234084_x_at | 8.91E−04 | — |
| 8998 | 216858_x_at | 9.00E−04 | — |
| 8998 | 217479_at | 9.25E−04 | — |
| 8998 | 1560638_a_at | 9.33E−04 | — |
| 8998 | 1569929_at | 0.001006 | — |
| 8998 | 1565705_x_at | 0.001007 | — |
| 8998 | 1557849_at | 0.00103 | — |
| 8998 | 1559709_at | 0.0011 | — |
| 8998 | 232775_at | 0.001301 | — |
| 8998 | 1565825_at | 0.001333 | — |
| 8998 | 1566609_at | 0.001347 | — |
| 8998 | 233478_at | 0.0015 | — |
| 8998 | 1561463_at | 0.0016 | — |
| 8998 | 241680_at | 0.002 | — |
| 8998 | 1568754_at | 0.002 | — |
| 8998 | 1563482_at | 0.002 | — |
| 8998 | 215128_at | 0.002204 | — |
| 8998 | 1555421_at | 1.01E−05 | AP1S3 |
| 8998 | 235647_at | 5.00E−04 | AP4S1 |
| 8998 | 212972_x_at | 0.002333 | APBB2 |
| 8998 | 203507_at | 6.78E−04 | CD68 |
| 8998 | 235918_x_at | 0.0014 | CEP97 |
| 8998 | 220084_at | 0.0035 | C14orf105 |
| 8998 | 217752_s_at | 0.0035 | CNDP2 |
| 8998 | 236900_x_at | 6.00E−04 | CCDC163P |
| 8998 | 1553811_at | 2.09E−04 | FAM222A-AS1 |
| 8998 | 204403_x_at | 1.50E−04 | FAM115A /// LOC100294033 |
| 8998 | 220896_at | 0.001015 | FBXL18 |
| 8998 | 211040_x_at | 3.53E−04 | GTSE1 |
| 8998 | 204318_s_at | 6.82E−04 | GTSE1 |
| 8998 | 214205_x_at | 2.76E−05 | GLRX3 |
| 8998 | 223563_at | 1.01E−05 | GNB1L |
| 8998 | 220081_x_at | 1.70E−04 | HSD17B7 |
| 8998 | 1553888_at | 1.01E−05 | LDHAL6A |
| 8998 | 1562392_at | 0.00115 | MRPL23 |
| 8998 | 214826_at | 7.00E−04 | PDE12 |
| 8998 | 214664_at | 2.00E−04 | PAICS |
| 8998 | 208828_at | 2.00E−04 | POLE3 |
| 8998 | 1553761_at | 4.60E−04 | PRR14L |
| 8998 | 215618_at | 0.001354 | RSU1 |
| 8998 | 1562428_at | 0.002 | LOC654780 |
| 8998 | 1570130_at | 1.75E−04 | SPATS2 |
| 8998 | 206438_x_at | 3.35E−05 | TCTN2 |
| 8998 | 223771_at | 1.67E−04 | TMEM87A |
| 8998 | 235097_at | 2.00E−04 | TRMT44 |
| 8998 | 220403_s_at | 8.50E−04 | TP53AIP1 |
| 8998 | 232420_x_at | 0.001 | LOC100289341 |
| 8998 | 1568722_at | 1.40E−05 | LOC100506832 |
| 8998 | 227940_at | 3.53E−04 | LOC339803 |
| 8998 | 1556609_at | 6.75E−04 | LOC401098 |
| 8998 | 232873_at | 3.90E−04 | ZNF33A |
| 8998 | 1552427_at | 9.00E−04 | ZNF485 |
| 8998 | 1558743_at | 0.001333 | ZNF620 |
| 9002 | 1563076_x_at | 2.00E−04 | — |
| 9002 | 1563075_s_at | 2.00E−04 | — |
| 9002 | 238701_x_at | 0.001 | C11orf92 |
| 9002 | 216288_at | 0.002 | CYSLTR1 |
| 9002 | 211481_at | 0.002 | SLCO1A2 |
| 9018 | 220509_at | 0.004 | RBM26 |
| 9035 | 217481_x_at | 1.05E−04 | — |
| 9035 | 1559795_at | 2.55E−04 | — |
| 9035 | 216642_at | 7.01E−04 | — |
| 9035 | 203989_x_at | 1.27E−04 | F2R |
| 9035 | 207115_x_at | 1.59E−04 | MBTD1 |
| 9035 | 209638_x_at | 5.15E−04 | RGS12 |
| 9035 | 233242_at | 0.001175 | WDR73 |
| 9035 | 241950_at | 3.66E−05 | WWC1 |
| 9040 | 206515_at | 4.00E−04 | CYP4F3 |
| 9040 | 220138_at | 0.005 | HAND1 |
| 9040 | 204735_at | 4.00E−04 | PDE4A |
| 9040 | 220984_s_at | 0.0015 | SLCO5A1 |
| 9040 | 1570006_at | 4.00E−04 | LOC400958 |
| 9051 | 235399_at | 3.00E−05 | — |
| 9051 | 1563345_at | 0.002008 | — |
| 9053 | 1562036_at | 4.00E−05 | — |
| 9056 | 244621_x_at | 0.003 | — |
| 9056 | 219223_at | 0.003 | CACFD1 |
| 9056 | 203787_at | 3.00E−04 | SSBP2 |
| 5944 | 1559682_at | 3.00E−04 | — |
| 5944 | 1560177_at | 2.00E−05 | CCDC66 |
| 5944 | 216768_x_at | 3.62E−04 | TTC38 |
| 5945 | 1566176_at | 0.0035 | — |
| 890 | 233289_at | 1.15E−04 | — |
| 890 | 1570127_at | 9.23E−04 | — |
| 890 | 1555375_at | 7.00E−05 | GRIK2 |
| 890 | 234341_x_at | 4.01E−05 | LOC91548 |
| 915 | 233154_at | 3.50E−04 | — |
| 915 | 1568892_at | 6.59E−04 | — |
| 915 | 238382_x_at | 6.67E−04 | — |
| 915 | 222299_x_at | 6.67E−04 | — |
| 915 | 234059_at | 6.93E−04 | — |
| 915 | 1566480_x_at | 3.30E−04 | C17orf104 |
| 915 | 221898_at | 0.001048 | PDPN |
| 921 | 217591_at | 2.16E−05 | — |
| 921 | 1564840_at | 2.88E−05 | — |
| 921 | 1557751_at | 9.83E−05 | — |
| 921 | 1570249_x_at | 2.02E−04 | — |
| 921 | 232468_at | 3.33E−04 | — |
| 921 | 233427_x_at | 4.32E−04 | — |
| 921 | 1561604_at | 4.81E−04 | — |
| 921 | 217643_x_at | 5.17E−04 | — |
| 921 | 233081_at | 5.38E−04 | — |
| 921 | 217288_at | 6.13E−04 | — |
| 921 | 215182_x_at | 6.13E−04 | — |
| 921 | 243167_at | 1.61E−04 | ABCB5 |
| 921 | 220600_at | 0.001033 | C3orf75 |
| 921 | 228700_at | 6.60E−04 | CXorf38 |
| 921 | 211286_x_at | 4.04E−06 | CSF2RA |
| 921 | 1556821_x_at | 8.33E−04 | DLEU2 |
| 921 | 1556820_a_at | 8.33E−04 | DLEU2 |
| 921 | 213475_s_at | 5.38E−04 | ITGAL |
| 921 | 221621_at | 7.61E−05 | LINC00338 /// SCARNA16 |
| 921 | 232034_at | 5.33E−04 | LINC00537 |
| 921 | 210722_at | 5.25E−04 | PNLIPRP1 |
| 921 | 235792_x_at | 2.52E−04 | PIK3C2A |
| 921 | 204461_x_at | 2.47E−04 | RAD1 |
| 921 | 217670_at | 0.00108 | RPLP2 |
| 921 | 1564972_x_at | 8.11E−04 | SETDB2 |
| 921 | 1564970_at | 8.11E−04 | SETDB2 |
| 921 | 206147_x_at | 4.04E−04 | SCML2 |
| 921 | 208157_at | 1.44E−05 | SIM2 |
| 921 | 1561080_at | 8.40E−04 | TXNRD1 |
| 921 | 207907_at | 4.02E−04 | TNFSF14 |
| 921 | 222280_at | 5.50E−04 | LOC100506469 |
| 921 | 239168_at | 4.51E−05 | LOC100652811 /// LOC100653083 /// LOC150622 /// LOC400940 |
| 921 | 208136_s_at | 6.87E−05 | MGC3771 |
| 921 | 1564362_x_at | 4.66E−04 | ZNF843 |
| 932 | 244533_at | 4.02E−04 | PTPN14 |
| 952 | 232496_at | 5.06E−04 | — |
| 952 | 210595_at | 6.82E−05 | ZNF235 |
| 980 | 1558965_at | 0.001 | PHF21A |
| 980 | 222564_at | 0.001 | POGK |
| 984 | 244620_at | 4.00E−10 | SLC8A1-AS1 |
| 1001 | 214989_x_at | 5.05E−04 | — |
| 5968 | 234762_x_at | 9.42E−04 | NLN |
| 1033 | 242859_at | 5.50E−04 | — |
| 1033 | 1561555_at | 6.26E−04 | — |
| 1033 | 1557522_x_at | 6.84E−04 | — |
| 1033 | 220856_x_at | 7.00E−04 | — |
| 1033 | 217698_at | 0.00125 | — |
| 1033 | 239222_at | 5.22E−04 | C9orf9 |
| 1033 | 217598_at | 2.56E−04 | CINP |
| 1033 | 212959_s_at | 4.57E−04 | GNPTAB |
| 1038 | 216337_at | 8.00E−05 | — |
| 1038 | 241722_x_at | 8.00E−05 | — |
| 1038 | 233376_at | 1.00E−04 | — |
| 1038 | 209770_at | 4.03E−05 | BTN3A1 |
| 1038 | 1556588_at | 2.00E−07 | C15orf37 |
| 1038 | 220180_at | 4.00E−08 | CCDC68 |
| 1038 | 235280_at | 4.00E−08 | POLR1A |
| 1043 | 1562673_at | 5.14E−04 | — |
| 1047 | 217610_at | 3.50E−04 | SPDYE2 |
| 1056 | 1565641_at | 0.0035 | C16orf45 |
| 1086 | 216463_at | 2.75E−04 | — |
| 1129 | 242589_x_at | 0.004 | — |
| 1138 | 215852_x_at | 0.00117 | SOGA1 |
| 1145 | 1558623_at | 1.00E−07 | LOC729121 |
| 1194 | 1558237_x_at | 2.00E−05 | — |
| 1221 | 205565_s_at | 0.001 | FXN |
| 1231 | 234920_at | 9.42E−04 | ZNF7 |
| 1231 | 223607_x_at | 0.005 | ZSWIM1 |
| 1252 | 225363_at | 4.00E−04 | PTEN |
| 1280 | 204028_s_at | 2.00E−07 | RABGAP1 |
| 1286 | 210138_at | 0.003 | RGS20 |
| 1286 | 205497_at | 0.003 | ZNF175 |
| 1302 | 1570151_at | 0.004 | — |
| 1302 | 1558489_at | 0.004 | — |
| 1302 | 1560754_at | 0.004 | CMTM7 |
| 1314 | 1555798_at | 0.001 | LOC400692 |
| 1339 | 1555374_at | 1.00E−05 | — |
| 1339 | 242371_x_at | 1.00E−04 | — |
| 1339 | 233055_at | 5.05E−04 | — |
| 1339 | 1564937_at | 6.14E−04 | — |
| 1339 | 215529_x_at | 9.33E−05 | DIP2A |
| 1339 | 242195_x_at | 0.00145 | NUMBL |
| 1340 | 241979_x_at | 0.004 | — |
| 1353 | 216146_at | 2.00E−04 | — |
| 1353 | 244088_at | 0.002 | — |
| 1353 | 1569321_at | 0.002 | — |
| 1353 | 1557542_at | 0.002 | — |
| 1390 | 239321_at | 0.003 | LOC441454 |
| 1394 | 1564568_at | 2.00E−04 | — |
| 1394 | 241268_x_at | 3.35E−04 | — |
| 1394 | 234439_at | 0.002 | — |
| 1407 | 240978_at | 3.40E−04 | — |
| 6009 | 242675_x_at | 0.002 | — |
| 1425 | 1559508_at | 3.55E−04 | — |
| 1425 | 1566232_at | 4.00E−04 | — |
| 1428 | 216793_x_at | 7.54E−04 | — |
| 1428 | 203904_x_at | 1.58E−04 | CD82 |
| 1428 | 235202_x_at | 2.91E−04 | IKBIP |
| 1446 | 230739_at | 0.002333 | FAM210A |
| 1449 | 1556797_at | 0.00225 | RNF144A-AS1 |
| 1468 | 1555400_at | 9.80E−05 | LOC645261 |
| 1471 | 1569537_at | 0.004 | — |
| 1501 | 232800_at | 4.00E−04 | — |
| 1501 | 216101_at | 0.001 | — |
| 1501 | 1560219_at | 0.001334 | ASB3 |
| 1510 | 244253_at | 0.003 | — |
| 1515 | 210723_x_at | 0.001025 | — |
| 1516 | 242842_at | 8.00E−04 | — |
| 1516 | 1557584_at | 0.003 | — |
| 1516 | 223431_at | 2.00E−04 | CNO |
| 1516 | 1553150_at | 0.0017 | KDM1B |
| 1516 | 227979_at | 8.00E−04 | RBM4 |
| 1516 | 1560679_at | 8.00E−04 | LOC100506328 |
| 1562 | 1570578_at | 6.00E−04 | — |
| 1562 | 235834_at | 6.00E−04 | CALD1 |
| 1562 | 227628_at | 0.001334 | GPX8 |
| 1562 | 235279_at | 2.00E−04 | PARVA |
| 1562 | 1563680_at | 2.62E−04 | LOC284950 |
| 9164 | 232054_at | 0.003 | PCDH20 |
| 9202 | 234805_at | 1.82E−04 | — |
| 9276 | 237853_x_at | 9.00E−04 | KRTAP10-12 |
| 9295 | 223019_at | 0.004 | FAM129B |
| 9320 | 1554849_at | 2.63E−04 | TOR1A |
| 9330 | 203898_at | 5.77E−04 | CRCP |
| 9330 | 1561532_at | 1.01E−05 | DACT3-AS1 |
| 9338 | 1558028_x_at | 0.001209 | LOC647979 |
| 9345 | 1554608_at | 6.46E−04 | TGOLN2 |
| 9347 | 236060_at | 9.13E−04 | — |
| 9347 | 233868_x_at | 0.00135 | ADAM33 |
| 9347 | 214782_at | 0.001348 | CTTN |
| 9347 | 242472_x_at | 1.52E−04 | FNBP4 |
| 9347 | 216188_at | 9.13E−04 | MYCNOS |
| 9347 | 219975_x_at | 4.61E−04 | OLAH |
| 9347 | 217049_x_at | 0.00117 | PCDH11X /// PCDH11Y |
| 9347 | 1570528_at | 0.002033 | XYLT2 |
| 9352 | 1564155_x_at | 0.001164 | — |
| 9352 | 1564154_at | 0.001164 | — |
| 9352 | 220039_s_at | 0.001601 | CDKAL1 |
| 9352 | 1566208_at | 2.00E−04 | TCEA1 |
| 9352 | 229587_at | 2.00E−04 | UBA2 |
| 9354 | 215595_x_at | 3.81E−04 | — |
| 9365 | 1559683_at | 1.05E−05 | — |
| 9365 | 234156_at | 2.33E−05 | — |
| 9365 | 217423_at | 2.50E−05 | — |
| 9365 | 222356_at | 2.00E−04 | — |
| 9365 | 1563934_at | 2.16E−04 | — |
| 9365 | 222170_at | 3.71E−04 | — |
| 9365 | 217616_at | 5.00E−04 | — |
| 9365 | 232840_at | 5.01E−04 | — |
| 9365 | 209155_s_at | 5.20E−04 | NT5C2 |
| 9365 | 1554246_at | 3.52E−04 | C1orf210 |
| 9365 | 233645_s_at | 2.00E−04 | C1RL |
| 9365 | 1560043_at | 1.00E−06 | CYB5R1 |
| 9365 | 213631_x_at | 2.97E−04 | DHODH |
| 9365 | 1564473_at | 4.51E−04 | ESCO2 |
| 9365 | 214124_x_at | 0.0011 | FGFR1OP |
| 9365 | 212221_x_at | 3.08E−04 | IDS |
| 9365 | 203943_at | 7.00E−04 | KIF3B |
| 9365 | 214675_at | 2.46E−04 | NUP188 |
| 9365 | 215863_at | 1.25E−04 | TFR2 |
| 9365 | 244716_x_at | 4.38E−04 | TMIGD2 |
| 9365 | 219589_s_at | 1.25E−04 | TMEM143 |
| 9365 | 244495_x_at | 8.13E−04 | TMEM241 |
| 9365 | 236715_x_at | 6.64E−04 | UACA |
| 9365 | 221626_at | 5.00E−05 | ZNF506 |
| 9385 | 1564767_at | 3.27E−04 | — |
| 9385 | 1569365_a_at | 0.002 | KIAA1656 |
| 9385 | 1554468_s_at | 0.00245 | MRPL38 |
| 9385 | 221859_at | 0.004 | SYT13 |
| 9385 | 1560390_s_at | 0.004 | LOC284440 |
| 9385 | 1554388_at | 7.67E−04 | VPS29 |
| 9386 | 215361_at | 3.00E−06 | DOCK5 |
| 9386 | 208674_x_at | 3.64E−04 | DDOST |
| 9386 | 231959_at | 5.00E−06 | LIN52 |
| 9386 | 223770_x_at | 8.24E−05 | MRI1 |
| 9386 | 1562321_at | 6.00E−06 | PDK4 |
| 9386 | 206487_at | 5.54E−04 | SUN1 |
| 9386 | 1558888_x_at | 0.00109 | ZNF321P /// ZNF816 /// ZNF816-ZNF321P |
| 9386 | 208137_x_at | 3.25E−04 | ZNF611 |
| 9386 | 220760_x_at | 5.00E−06 | ZNF665 |
| 9390 | 215233_at | 3.44E−04 | JMJD6 |
| 9397 | 210599_at | 3.00E−12 | ZNF614 |
| 9402 | 222888_at | 1.00E−04 | CCNJ |
| 5636 | 215439_x_at | 3.77E−04 | — |
| 1595 | 222206_s_at | 0.005 | NCLN |
| 1599 | 1557151_at | 3.00E−04 | — |
| 1599 | 243642_x_at | 6.00E−04 | — |
| 1599 | 1566777_at | 0.001 | — |
| 1604 | 222864_s_at | 0.001 | ZNF219 |
| 1611 | 207734_at | 0.004 | LAX1 |
| 1711 | 1560917_at | 9.01E−06 | — |
| 1711 | 215397_x_at | 1.50E−05 | — |
| 1711 | 215406_at | 1.60E−04 | — |
| 1711 | 216645_at | 2.42E−04 | — |
| 1711 | 1562265_at | 2.44E−04 | — |
| 1711 | 214912_at | 2.77E−04 | — |
| 1711 | 1557842_at | 2.90E−04 | — |
| 1711 | 215370_at | 3.34E−04 | — |
| 1711 | 233553_at | 3.34E−04 | — |
| 1711 | 1558078_at | 3.35E−04 | — |
| 1711 | 1563519_at | 5.50E−04 | — |
| 1711 | 1554972_at | 6.00E−04 | — |
| 1711 | 1556327_a_at | 7.51E−04 | — |
| 1711 | 215650_at | 7.86E−04 | — |
| 1711 | 207915_at | 8.00E−04 | — |
| 1711 | 1559210_at | 8.63E−04 | — |
| 1711 | 216527_at | 0.001 | — |
| 1711 | 216198_at | 1.00E−05 | ATF7IP |
| 1711 | 215590_x_at | 2.00E−05 | ACVR2B-AS1 |
| 1711 | 1558792_x_at | 0.001006 | AP2A1 |
| 1711 | 1553253_at | 0.001 | ASB16 |
| 1711 | 215365_at | 0.0015 | CACNB2 |
| 1711 | 1557801_x_at | 0.00105 | C11orf31 |
| 1711 | 1557799_at | 0.00105 | C11orf31 |
| 1711 | 214816_x_at | 3.10E−04 | C19orf40 |
| 1711 | 232266_x_at | 8.17E−04 | CDK13 |
| 1711 | 210965_x_at | 0.00155 | CDK13 |
| 1711 | 202577_s_at | 0.001333 | DDX19A |
| 1711 | 204211_x_at | 2.17E−04 | EIF2AK2 |
| 1711 | 216786_at | 9.63E−04 | FAM224A |
| 1711 | 215602_at | 4.00E−05 | FGD2 |
| 1711 | 211424_x_at | 1.22E−04 | METTL7A |
| 1711 | 209703_x_at | 1.22E−04 | METTL7A |
| 1711 | 205989_s_at | 8.06E−04 | MOG |
| 1711 | 215850_s_at | 1.03E−04 | NDUFA5 |
| 1711 | 228676_at | 1.75E−04 | ORAOV1 |
| 1711 | 217624_at | 6.90E−04 | PDAP1 |
| 1711 | 219392_x_at | 1.86E−04 | PRR11 |
| 1711 | 226090_x_at | 0.001377 | RABL3 |
| 1711 | 217775_s_at | 1.00E−05 | RDH11 |
| 1711 | 1559882_at | 8.00E−05 | SAMHD1 |
| 1711 | 215889_at | 4.00E−04 | SKIL |
| 1711 | 205255_x_at | 4.67E−04 | TCF7 |
| 1711 | 214815_at | 5.00E−04 | TRIM33 |
| 1711 | 214817_at | 4.03E−04 | UNC13A |
| 1711 | 232731_x_at | 5.13E−04 | LOC100190938 |
| 1711 | 220401_at | 0.001158 | FLJ21369 |
| 1711 | 207090_x_at | 4.67E−04 | ZFP30 |
| 1717 | 1569732_at | 4.55E−06 | — |
| 1717 | 233038_at | 3.81E−04 | — |
| 1717 | 223815_at | 2.28E−04 | CEP95 |
| 1717 | 224672_x_at | 3.60E−04 | MESDC2 |
| 1718 | 234170_at | 6.00E−04 | — |
| 1718 | 1564665_at | 0.003 | — |
| 1718 | 244052_at | 0.001 | CBR4 |
| 1718 | 206247_at | 0.001325 | MICB |
| 1718 | 1559705_s_at | 0.001001 | PHKA2 |
| 1718 | 216161_at | 9.50E−04 | SBNO1 |
| 1718 | 1561059_a_at | 6.67E−04 | LOC152024 |
| 1723 | 200905_x_at | 3.10E−05 | HLA-E |
| 1723 | 210389_x_at | 5.00E−06 | TUBD1 |
| 1725 | 1557996_at | 7.67E−04 | — |
| 1732 | 1562898_at | 2.55E−04 | — |
| 1772 | 235381_at | 0.002 | RSF1 |
| 1776 | 1554786_at | 1.00E−05 | CASS4 |
| 1782 | 223335_at | 4.00E−07 | TMEM69 |
| 1792 | 1564449_at | 6.23E−04 | — |
| 1792 | 216163_at | 7.60E−04 | — |
| 1792 | 243901_at | 5.60E−04 | ALDOB |
| 1792 | 211277_x_at | 8.58E−04 | APP |
| 1792 | 207262_at | 6.85E−04 | APOF |
| 1792 | 206775_at | 0.00156 | CUBN |
| 1792 | 205370_x_at | 4.66E−04 | DBT |
| 1792 | 222733_x_at | 7.58E−04 | RRP1 |
| 1792 | 206057_x_at | 0.001388 | SPN |
| 1792 | 216981_x_at | 0.001388 | SPN |
| 1792 | 1565771_at | 0.001206 | DKFZp434E1119 |
| 1792 | 242633_x_at | 9.50E−04 | ZNF75A |
| 1797 | 241835_at | 0.004 | — |
| 1842 | 232106_s_at | 4.67E−04 | CEP89 |
| 1859 | 1566716_at | 3.01E−04 | — |
| 1885 | 234627_at | 0.001683 | FLJ21408 |
| 1889 | 1569542_at | 5.10E−04 | — |
| 1889 | 228141_at | 4.27E−04 | GPX8 |
| 1889 | 238489_at | 0.0025 | PRKAA2 |
| 1889 | 1559126_at | 0.001 | RRP12 |
| 1890 | 207481_at | 0.001231 | — |
| 1891 | 232803_at | 2.65E−04 | — |
| 1891 | 244197_x_at | 5.00E−04 | — |
| 1891 | 232797_at | 5.00E−04 | — |
| 1891 | 220144_s_at | 1.00E−04 | ANKRD5 |
| 1891 | 228813_at | 5.00E−04 | HDAC4 |
| 1891 | 1553808_a_at | 0.002 | NKX2-3 |
| 1891 | 219105_x_at | 0.001036 | ORC6 |
| 1891 | 1558365_at | 5.00E−04 | LOC100652805 /// LOC100653302 /// PGK1 |
| 1891 | 1553354_a_at | 1.00E−04 | FLJ31958 |
| 1951 | 1558402_at | 9.45E−04 | — |
| 1955 | 242448_at | 8.00E−05 | — |
| 1955 | 217580_x_at | 0.001 | — |
| 1955 | 217579_x_at | 0.001 | — |
| 1955 | 231880_at | 0.003 | FAM40B |
| 1955 | 218413_s_at | 0.003 | ZNF639 |
| 98 | 217537_x_at | 6.00E−05 | — |
| 98 | 234266_at | 1.67E−04 | — |
| 98 | 217490_at | 2.69E−04 | — |
| 98 | 216496_s_at | 5.00E−04 | — |
| 98 | 220689_at | 5.00E−04 | — |
| 98 | 216769_x_at | 6.38E−04 | — |
| 98 | 241724_x_at | 8.96E−04 | — |
| 98 | 233448_s_at | 9.19E−04 | — |
| 98 | 216179_x_at | 0.001027 | — |
| 98 | 1570013_at | 0.001301 | — |
| 98 | 1563561_at | 0.001757 | — |
| 98 | 1553234_at | 1.75E−04 | ADAMTS18 |
| 98 | 1570128_at | 3.34E−04 | DDX19A |
| 98 | 1568884_at | 0.001067 | DNAJC2 |
| 98 | 219618_at | 3.54E−04 | IRAK4 |
| 98 | 243539_at | 3.02E−04 | KIAA1841 |
| 98 | 1552979_at | 7.24E−04 | LINC00471 |
| 98 | 1559167_x_at | 0.001125 | MPV17L |
| 98 | 236860_at | 6.67E−04 | NPY6R |
| 98 | 1553364_at | 0.00123 | PNPLA1 |
| 98 | 202739_s_at | 5.98E−04 | PHKB |
| 98 | 1556308_at | 6.88E−04 | PRRT3 |
| 98 | 217348_x_at | 2.56E−04 | ARHGEF15 |
| 98 | 222121_at | 5.67E−04 | ARHGEF26 |
| 98 | 233903_s_at | 5.67E−04 | ARHGEF26 |
| 98 | 210760_x_at | 4.15E−04 | TRIP11 |
| 98 | 1565723_at | 3.50E−04 | LOC100128281 |
| 98 | 207263_x_at | 0.00107 | VEZT |
| 98 | 232303_at | 2.25E−05 | ZNF608 |
| 101 | 1569999_at | 0.004 | — |
| 2012 | 233273_at | 0.00155 | — |
| 2035 | 215768_at | 0.001 | — |
| 2035 | 1566637_at | 0.001 | — |
| 2035 | 1564007_at | 0.001 | — |
| 2035 | 1561192_at | 0.001 | — |
| 2035 | 216144_at | 0.001252 | — |
| 2035 | 1566459_at | 0.001295 | — |
| 2035 | 233906_at | 0.003 | — |
| 2035 | 209774_x_at | 0.001 | CXCL2 |
| 2035 | 1561190_at | 0.001067 | CDKL3 |
| 2035 | 201313_at | 0.003 | ENO2 |
| 2035 | 209193_at | 0.003 | PIM1 |
| 2035 | 1553717_at | 5.25E−04 | KCTD7 /// RABGEF1 |
| 2035 | 1563581_at | 5.69E−04 | RPL34-AS1 |
| 2035 | 204743_at | 0.0025 | TAGLN3 |
| 2035 | 237745_at | 0.001 | TSC22D1-AS1 |
| 2035 | 229930_at | 0.001 | LOC100134361 |
| 2035 | 1569318_at | 0.001 | LOC284440 |
| 2045 | 202464_s_at | 0.001 | PFKFB3 |
| 2049 | 202015_x_at | 2.52E−06 | — |
| 2080 | 1569192_at | 1.01E−04 | — |
| 2080 | 215204_at | 4.78E−04 | — |
| 2080 | 216103_at | 0.003 | ACOT11 |
| 2080 | 207536_s_at | 4.44E−04 | TNFRSF9 |
| 2108 | 232754_at | 0.0025 | — |
| 2110 | 215587_x_at | 5.91E−04 | — |
| 2113 | 221815_at | 2.00E−06 | ABHD2 |
| 2124 | 233931_at | 1.48E−04 | — |
| 115 | 234491_s_at | 0.0025 | SAV1 |
| 2135 | 1561571_at | 1.00E−07 | LOC730139 |
| 2136 | 1566183_at | 0.00124 | — |
| 2136 | 204842_x_at | 0.001733 | PRKAR2A |
| 2146 | 1562297_at | 0.004 | — |
| 2191 | 207524_at | 0.002 | ST7 /// ST7-OT3 |
| 123 | 234060_at | 0.001244 | — |
| 2219 | 1558595_at | 0.003 | — |
| 125 | 1557004_at | 2.00E−14 | LOC100505902 /// LOC285389 |
| 2228 | 222006_at | 0.001667 | LETM1 |
| 130 | 209246_at | 0.001167 | ABCF2 |
| 130 | 233254_x_at | 0.004 | PTEN |
| 130 | 205628_at | 3.00E−04 | PRIM2 |
| 2270 | 243310_at | 2.00E−04 | — |
| 2270 | 1569983_at | 2.60E−05 | LINC00566 |
| 2270 | 1560202_at | 4.00E−06 | LOC338739 |
| 2322 | 1557538_at | 0.001317 | — |
| 2333 | 238826_x_at | 0.001 | — |
| 2333 | 203742_s_at | 0.001 | LOC732360 /// TDG |
| 2333 | 234332_at | 0.002004 | NUB1 |
| 2333 | 1569030_s_at | 0.002004 | NUB1 |
| 2340 | 1569566_at | 0.001 | TBC1D1 |
| 139 | 1561432_at | 7.00E−04 | — |
| 139 | 233341_s_at | 2.66E−04 | POLR1B |
| 2365 | 216524_x_at | 0.001338 | — |
| 2365 | 1559206_at | 4.02E−04 | PSEN1 |
| 2379 | 243876_at | 3.00E−04 | — |
| 2379 | 1560451_at | 3.65E−04 | — |
| 2379 | 1566768_at | 5.00E−04 | — |
| 2379 | 222370_x_at | 5.01E−04 | — |
| 2379 | 216472_at | 5.35E−04 | — |
| 2379 | 215197_at | 5.44E−04 | — |
| 2379 | 232665_x_at | 6.61E−04 | — |
| 2379 | 234833_at | 6.67E−04 | — |
| 2379 | 215445_x_at | 7.54E−04 | — |
| 2379 | 1562853_x_at | 8.33E−04 | — |
| 2379 | 1562852_at | 8.33E−04 | — |
| 2379 | 1560971_a_at | 0.001001 | — |
| 2379 | 1555014_x_at | 0.001013 | — |
| 2379 | 233239_at | 0.001041 | — |
| 2379 | 1560560_at | 0.001067 | — |
| 2379 | 210698_at | 0.001258 | — |
| 2379 | 233017_x_at | 0.00129 | — |
| 2379 | 215083_at | 0.002 | — |
| 2379 | 1565877_at | 0.002175 | — |
| 2379 | 1565876_x_at | 0.002175 | — |
| 2379 | 242113_at | 0.0023 | — |
| 2379 | 235716_at | 0.003 | — |
| 2379 | 1555321_at | 0.002 | ACOT11 |
| 2379 | 233182_x_at | 0.00109 | ATXN3 |
| 2379 | 1555323_at | 0.00105 | ABCB9 |
| 2379 | 1557963_at | 2.00E−04 | CDC42BPB |
| 2379 | 209057_x_at | 6.98E−04 | CDC5L |
| 2379 | 215072_x_at | 0.001205 | C10orf137 |
| 2379 | 208115_x_at | 0.001205 | C10orf137 |
| 2379 | 238028_at | 3.75E−04 | C6orf132 |
| 2379 | 1553446_at | 0.002 | C6orf183 |
| 2379 | 206661_at | 5.15E−04 | DBF4B |
| 2379 | 1559039_at | 0.0035 | DHX36 |
| 2379 | 208386_x_at | 4.88E−04 | DMC1 |
| 2379 | 211328_x_at | 0.002 | HFE |
| 2379 | 239177_at | 0.0025 | IRGQ |
| 2379 | 1559142_at | 3.01E−04 | KAT6A |
| 2379 | 215375_x_at | 0.00118 | LRRFIP1 |
| 2379 | 1554582_a_at | 5.15E−04 | METTL20 |
| 2379 | 232168_x_at | 0.00101 | MACF1 |
| 2379 | 225942_at | 0.002 | NLN |
| 2379 | 1568913_at | 7.34E−04 | NSUN3 |
| 2379 | 210636_at | 3.64E−04 | PPARD |
| 2379 | 203555_at | 0.0016 | PTPN18 |
| 2379 | 1560322_at | 2.53E−04 | RBMS3 |
| 2379 | 1568964_x_at | 9.38E−04 | SPN |
| 2379 | 1553011_at | 7.96E−04 | TAF1L |
| 2379 | 1566207_at | 0.00125 | TCEA1 |
| 2379 | 217974_at | 0.0025 | TM7SF3 |
| 2379 | 208099_x_at | 0.003 | TTLL5 |
| 2379 | 1569604_at | 1.79E−04 | LOC100133612 |
| 2379 | 1562095_at | 4.63E−04 | LOC100287789 /// SLMAP |
| 2379 | 211386_at | 0.0025 | MGC12488 |
| 2383 | 236618_at | 1.00E−08 | C20orf132 |
| 2383 | 214601_at | 2.00E−04 | TPH1 |
| 2413 | 240356_s_at | 6.00E−04 | — |
| 2497 | 217586_x_at | 7.80E−04 | — |
| 2497 | 230808_at | 4.34E−04 | FNTA |
| 2497 | 215436_at | 0.0011 | HSDL2 |
| 2497 | 227277_at | 0.0023 | MTDII |
| 2497 | 1559849_at | 9.73E−04 | ZNF605 |
| 2524 | 214902_x_at | 0.001231 | — |
| 2524 | 205096_at | 8.40E−04 | POM121 |
| 2555 | 226212_s_at | 0.004 | INSR |
| 2603 | 208818_s_at | 0.003 | COMT |
| 2603 | 208817_at | 0.003 | COMT |
| 2619 | 1558906_a_at | 3.00E−05 | LOC100505783 |
| 2623 | 215753_at | 9.00E−04 | — |
| 2623 | 216499_at | 0.0015 | — |
| 2623 | 1561451_a_at | 0.001226 | ADAM29 |
| 2623 | 1555981_at | 8.33E−04 | C17orf65 |
| 2623 | 231781_s_at | 8.00E−04 | LRRC2 |
| 2623 | 221605_s_at | 9.19E−04 | PIPOX |
| 2637 | 1565889_at | 0.001533 | — |
| 2637 | 1565888_at | 0.001533 | — |
| 2637 | 242457_at | 0.00215 | — |
| 2651 | 1557232_at | 0.001018 | — |
| 170 & 171 | 235105_at | 0.0021 | — |
| 2723 | 1556595_at | 0.004 | — |
| 2723 | 1559069_at | 0.004 | MTHFS /// ST20-MTHFS |
| 2723 | 206679_at | 0.004 | APBA1 |
| 2723 | 1558868_a_at | 0.004 | DSE |
| 2730 | 235608_at | 0.002 | — |
| 2747 | 1562454_at | 1.00E−04 | — |
| 2747 | 1559420_x_at | 1.00E−04 | CACNB2 |
| 2747 | 1559419_at | 1.00E−04 | CACNB2 |
| 2750 | 1562890_at | 3.05E−04 | — |
| 2750 | 232723_at | 6.44E−04 | — |
| 2750 | 227551_at | 4.65E−04 | FAM108B1 |
| 2750 | 206681_x_at | 3.68E−04 | GP2 |
| 2750 | 1558832_at | 4.67E−04 | SLC2A1-AS1 |
| 2750 | 241643_at | 1.03E−05 | TLK1 |
| 2750 | 241642_x_at | 4.08E−04 | TLK1 |
| 2750 | 1559576_at | 2.00E−06 | WDR78 |
| 2750 | 1559616_x_at | 8.44E−04 | ZNF626 |
| 2779 | 218923_at | 2.00E−04 | CTBS |
| 2785 | 216309_x_at | 0.001 | JRK |
| 2785 | 214692_s_at | 0.001 | JRK |
| 185 | 1561234_at | 6.67E−05 | — |
| 185 | 238570_at | 8.00E−04 | — |
| 185 | 220812_s_at | 6.67E−05 | HHLA2 |
| 185 | 233997_at | 6.67E−05 | LOC100287166 |
| 2824 | 242813_at | 6.43E−04 | — |
| 187 | 233128_at | 0.003 | — |
| 187 | 1563332_at | 0.003 | — |
| 187 | 234737_at | 0.002 | NT5DC3 |
| 187 | 220233_at | 3.00E−04 | FBXO17 /// SARS2 |
| 187 | 242578_x_at | 0.003 | SLC22A3 |
| 2829 | 204861_s_at | 6.00E−10 | LOC100509323 /// NAIP |
| 2829 | 231772_x_at | 2.00E−09 | CENPH |
| 2835 | 1553072_at | 3.00E−08 | BNIPL |
| 2835 | 209941_at | 1.00E−05 | RIPK1 |
| 2860 | 233027_at | 0.001287 | — |
| 2860 | 1560868_s_at | 0.001505 | — |
| 2860 | 238912_x_at | 0.003 | C9orf85 |
| 2860 | 222265_at | 0.00105 | TNS4 |
| 2860 | 215455_at | 0.0025 | TIMELESS |
| 2860 | 232918_at | 0.001043 | LOC541471 |
| 191 | 1564294_at | 1.45E−04 | — |
| 191 | 1564794_at | 2.00E−04 | — |
| 191 | 1564789_at | 2.00E−04 | — |
| 191 | 1559436_x_at | 2.00E−04 | — |
| 191 | 1556176_at | 2.00E−04 | TAF8 |
| 2899 | 204746_s_at | 0.004 | PICK1 |
| 2981 | 1566043_at | 0.002045 | — |
| 2991 | 1569948_at | 6.00E−04 | — |
| 2991 | 219380_x_at | 1.00E−04 | POLH |
| 208 | 204845_s_at | 0.003 | ENPEP |
| 2946 | 1566482_at | 0.005 | — |
| 2946 | 224707_at | 0.005 | CYSTM1 |
| 2952 | 219058_x_at | 9.61E−04 | TINAGL1 |
| 211 | 238656_at | 4.00E−04 | — |
| 3081 | 233608_at | 2.67E−04 | — |
| 3081 | 224134_at | 0.002 | MGC10814 |
| 3095 | 202495_at | 0.004 | TBCC |
| 3102 | 219605_at | 4.00E−04 | ZNF3 |
| 3110 | 210486_at | 0.001028 | ANKMY1 |
| 3143 | 1564463_at | 0.001038 | LOC100499221 |
| 3145 | 224227_s_at | 2.70E−23 | BDP1 |
| 3210 | 1553910_at | 1.00E−21 | NBFF4 |
| 3290 | 232622_at | 2.65E−04 | — |
| 3294 | 230118_at | 1.68E−04 | — |
| 3294 | 222247_at | 2.50E−04 | — |
| 3294 | 1563154_at | 2.50E−04 | — |
| 3354 | 242024_at | 5.00E−06 | — |
| 3354 | 242117_at | 6.00E−05 | — |
| 3354 | 232119_at | 6.00E−05 | SYNPO2 |
| 3363 | 215541_s_at | 5.00E−20 | DIAPH1 |
| 3395 | 1554266_at | 1.59E−04 | — |
| 3395 | 243568_at | 2.03E−04 | — |
| 3395 | 1556339_a_at | 2.26E−04 | — |
| 3395 | 241303_x_at | 3.77E−04 | — |
| 3395 | 222213_x_at | 6.06E−04 | — |
| 3395 | 240839_at | 8.83E−04 | — |
| 3395 | 220076_at | 4.55E−05 | ANKH |
| 3395 | 1561407_at | 1.35E−05 | ARAP2 |
| 3395 | 207986_x_at | 4.88E−05 | CYB561 |
| 3395 | 232250_at | 9.45E−04 | KIAA1257 |
| 3395 | 207492_at | 0.0012 | NGLY1 |
| 3395 | 206048_at | 3.70E−05 | OVOL2 |
| 3395 | 1552809_at | 4.50E−05 | RFX4 |
| 3395 | 220791_x_at | 4.00E−06 | SCN11A |
| 3395 | 232502_at | 1.26E−04 | LOC100652735 /// LOC100653122 /// LOC728558 |
| 3395 | 1561566_at | 1.71E−04 | LOC285501 |
| 3406 | 234222_at | 0.00141 | — |
| 3406 | 1561181_at | 0.0021 | — |
| 3406 | 203967_at | 0.001087 | CDC6 |
| 3406 | 239937_at | 0.001264 | ZNF207 |
| 3407 | 215137_at | 5.50E−04 | — |
| 3465 | 1562733_at | 5.00E−04 | LINC00092 |
| 3509 | 232335_at | 0.002538 | — |
| 3517 | 232215_x_at | 4.05E−05 | PRR11 |
| 3544 | 223781_x_at | 0.00205 | ADH4 |
| 3544 | 229549_at | 0.004 | CALU |
| 3544 | 204440_at | 0.00135 | CD83 |
| 3544 | 229021_at | 0.001336 | MCTP2 |
| 3545 | 234962_at | 0.003 | — |
| 3561 | 233181_at | 1.00E−04 | — |
| 3561 | 222290_at | 1.00E−04 | OR2A20P /// OR2A9P |
| 3561 | 224006_at | 2.00E−07 | LOC100507377 |
| 3588 | 227797_x_at | 0.002 | LYRM2 |
| 3592 | 1569942_at | 0.00102 | — |
| 3592 | 236571_at | 0.00165 | — |
| 3592 | 244047_at | 0.003 | — |
| 3592 | 243646_at | 0.003 | — |
| 3592 | 1559309_at | 0.003 | MCFD2 |
| 3592 | 239462_at | 0.003 | ZNF284 |
| 3595 | 1562071_at | 4.50E−06 | — |
| 3595 | 1554599_x_at | 4.50E−06 | — |
| 3595 | 233296_x_at | 1.77E−04 | — |
| 3595 | 231873_at | 4.50E−06 | BMPR2 |
| 3595 | 232442_at | 3.01E−08 | BCAR1 |
| 3595 | 1562777_at | 4.50E−06 | ERV3-1 |
| 3595 | 1555317_at | 1.50E−05 | POLK |
| 3595 | 223176_at | 0.001336 | KCTD20 |
| 3609 | 1559509_at | 4.00E−04 | — |
| 3609 | AFFX- | 5.16E−04 | — |
| 3609 | 232778_at | 0.001133 | — |
| 3609 | 1557468_at | 0.001305 | — |
| 3609 | 236566_at | 0.00325 | — |
| 3609 | 222710_at | 0.001211 | AMIGO3 /// GMPPB |
| 3609 | 211004_s_at | 1.67E−04 | ALDH3B1 |
| 3609 | 1558756_at | 0.001285 | C2CD3 |
| 3609 | 1553162_x_at | 3.34E−04 | C19orf55 |
| 3609 | 206497_at | 0.001043 | COA1 |
| 3609 | 219290_x_at | 4.03E−04 | DAPP1 |
| 3609 | 215810_x_at | 5.12E−04 | DST |
| 3609 | 215404_x_at | 0.001024 | FGFR1 |
| 3609 | 239924_at | 4.37E−04 | GUSBP11 |
| 3609 | 213835_x_at | 0.001021 | GTPBP3 |
| 3609 | 206551_x_at | 7.92E−04 | KLHL24 |
| 3609 | 235955_at | 6.00E−04 | MARVELD2 |
| 3609 | 215560_x_at | 5.43E−04 | MTRF1L |
| 3609 | 1568986_x_at | 8.95E−04 | PIGT |
| 3609 | 221596_s_at | 3.60E−04 | RBM48 |
| 3609 | 220091_at | 1.43E−04 | SLC2A6 |
| 3609 | 1556206_at | 0.001 | LOC100652856 |
| 3609 | 220983_s_at | 0.00184 | LOC100653017 /// MIR612 /// NEAT1 |
| 3609 | 1553704_x_at | 0.001089 | ZNF791 |
| 3609 | 1553703_at | 0.001089 | ZNF791 |
| 3620 | 220113_x_at | 6.54E−04 | POLR1B |
| 3626 | 235681_at | 3.00E−05 | — |
| 3626 | 1569459_a_at | 3.00E−05 | — |
| 3626 | 241888_at | 1.00E−04 | — |
| 3626 | 1565874_at | 4.00E−04 | — |
| 3626 | 233042_at | 0.001 | — |
| 3626 | 202532_s_at | 3.00E−05 | DHFR |
| 3626 | 241727_x_at | 4.00E−04 | DHFRL1 |
| 3626 | 228012_at | 3.00E−05 | MATR3 /// SNHG4 |
| 3626 | 1562682_at | 1.00E−04 | RORA |
| 3626 | 1566932_x_at | 2.00E−06 | TFB2M |
| 3626 | 1552302_at | 3.00E−05 | TMEM106A |
| 3646 | 221120_at | 9.00E−04 | — |
| 3646 | 214718_at | 0.001 | GATAD1 |
| 3653 | 206931_at | 0.004 | ZNF141 |
| 3676 | 1566740_at | 0.001244 | PLCE1 |
| 3697 | 1562811_at | 7.83E−04 | — |
| 3697 | 202897_at | 0.002 | SIRPA |
| 3716 | 224592_x_at | 0.001251 | HP1BP3 |
| 3716 | 223871_x_at | 0.00101 | ING5 |
| 3716 | 225746_at | 0.00115 | RAB11FIP4 |
| 3716 | 1564683_at | 0.001189 | TBL3 |
| 3716 | 218050_at | 0.001006 | UFM1 |
| 3728 | 201631_s_at | 0.003 | IER3 |
| 3738 | 232621_at | 5.00E−05 | USP48 |
| 3750 | 1557334_a_at | 0.004 | FERMT1 |
| 280 | 243877_at | 3.00E−04 | — |
| 285 | 234758_at | 8.00E−05 | — |
| 3817 | 215295_at | 0.003 | DTNB |
| 3847 | 223999_at | 0.002 | PPIL2 |
| 3857 | 211775_x_at | 1.23E−04 | MGC13053 |
| 293 | 227509_x_at | 0.0025 | — |
| 293 | 206926_s_at | 0.001 | IL11 |
| 293 | 242077_x_at | 0.0021 | MB21D1 |
| 3906 | 233300_at | 2.00E−04 | — |
| 3915 | 233635_at | 0.001 | — |
| 3915 | 1559932_at | 0.001 | — |
| 3915 | 1570082_x_at | 0.004 | — |
| 3937 | 222136_x_at | 0.001202 | ZNF43 |
| 3910 | 1569673_at | 0.00335 | — |
| 3952 | 1559277_at | 4.00E−10 | FLJ35700 |
| 3953 | 241223_x_at | 2.00E−04 | — |
| 3953 | 1558474_at | 2.00E−04 | — |
| 3953 | 1558385_at | 2.00E−04 | — |
| 3953 | 216675_at | 0.002 | — |
| 3953 | 1564816_at | 2.00E−04 | C14orf178 |
| 3953 | 215600_x_at | 2.00E−04 | FBXW12 |
| 3953 | 210528_at | 2.00E−04 | MR1 |
| 3953 | 210771_at | 2.00E−04 | PPARA |
| 3953 | 233123_at | 0.002 | SLC40A1 |
| 3953 | 216775_at | 2.00E−04 | USP53 |
| 3964 | 241135_at | 0.002 | — |
| 147 | 224284_x_at | 5.04E−05 | FKSG49 |
| 3992 | 1560297_at | 2.67E−04 | — |
| 3992 | 227967_at | 5.50E−06 | TUBGCP5 |
| 3993 | 203460_s_at | 0.003 | PSEN1 |
| 4012 | 1562793_at | 3.84E−04 | — |
| 4012 | 1556831_at | 6.00E−04 | DYNC1H1 |
| 4012 | 244391_at | 4.37E−04 | TSEN2 |
| 4042 | 242171_at | 0.003 | — |
| 4064 | 233120_at | 2.47E−06 | — |
| 4064 | 216630_at | 6.93E−06 | — |
| 4064 | 1569597_at | 7.50E−04 | — |
| 4064 | 220814_at | 8.13E−04 | — |
| 4064 | 224024_at | 0.001 | ERGIC1 |
| 4064 | 1557289_s_at | 7.56E−04 | GTF2IRD2 /// GTF2IRD2B |
| 4064 | 218202_x_at | 0.001313 | MRPL44 |
| 4064 | 1552476_s_at | 5.22E−04 | PLCD3 |
| 4064 | 216957_at | 4.33E−07 | USP22 |
| 4064 | 1569270_at | 2.50E−06 | LOC100134368 |
| 4064 | 232131_at | 0.001 | LOC100506874 |
| 4083 | 224478_s_at | 4.00E−11 | C7orf50 |
| 4085 | 215831_at | 6.54E−05 | — |
| 4147 | 224052_at | 0.001 | HSFY1 /// HSFY2 |
| 4170 | 235207_at | 9.01E−04 | — |
| 4170 | 1559895_x_at | 0.001343 | — |
| 4170 | 238363_at | 0.001367 | — |
| 4170 | 242990_at | 0.0017 | — |
| 4170 | 223759_s_at | 0.003 | GSG2 |
| 4170 | 217771_at | 0.003 | GOLM1 |
| 4170 | 242512_at | 0.0017 | MYO9A |
| 4170 | 241919_x_at | 0.003 | WDR31 |
| 4187 | 1561584_at | 0.003 | — |
| 4187 | 210096_at | 0.003 | CYP4B1 |
| 4187 | 1554335_at | 0.003 | CYTH4 |
| 4187 | 1559715_at | 0.003 | LOC100507391 |
| 4188 | 208120_x_at | 4.00E−06 | FKSG49 /// LOC100506290 /// LOC100653153 |
| 4218 | 217473_x_at | 0.0028 | SLC11A1 |
| 4218 | 210422_x_at | 0.0034 | SLC11A1 |
| 4220 | 1564004_at | 0.001 | — |
| 4220 | 1569767_at | 0.001002 | — |
| 4220 | 1570138_at | 0.001067 | — |
| 4220 | 215539_at | 0.002667 | — |
| 4220 | 233779_x_at | 0.003 | — |
| 4220 | 242444_at | 9.20E−04 | C1QTNF6 |
| 4220 | 218290_at | 0.001 | PLEKHJ1 |
| 4220 | 1563465_at | 5.99E−04 | PKD1L1 |
| 4220 | 208671_at | 3.40E−04 | SERINC1 |
| 4220 | 1564208_x_at | 3.41E−04 | FLJ35390 |
| 4220 | 1569090_x_at | 6.02E−04 | FLJ35390 |
| 4220 | 1569089_a_at | 3.41E−04 | FLJ35390 /// LOC100506683 |
| 4221 | 243025_at | 6.00E−04 | — |
| 4292 | 242451_x_at | 0.001433 | RPS19 |
| 10102 | 232597_x_at | 2.00E−05 | SCAF11 |
| 4256 | 1560690_at | 0.004 | — |
| 4267 | 215053_at | 0.004 | SRCAP |
| 4268 | 218735_s_at | 0.001434 | ZNF544 |
| 4270 | 238523_at | 8.00E−05 | KLHL36 |
| 4270 | 239796_x_at | 3.00E−04 | TIRAP |
| 4323 | 207796_x_at | 8.00E−04 | KLRD1 |
| 4324 | 207756_at | 0.001044 | — |
| 4324 | 1561597_x_at | 0.001262 | — |
| 4324 | 236327_at | 0.0024 | — |
| 4324 | 220719_at | 0.0025 | — |
| 4324 | 1564820_at | 0.004 | — |
| 4324 | 1561596_at | 0.004 | — |
| 4324 | 242553_at | 0.004 | ABCC3 |
| 4324 | 1553821_at | 0.001283 | C12orf51 |
| 4331 | 217570_x_at | 3.36E−04 | — |
| 4331 | 1562541_at | 4.20E−04 | — |
| 4331 | 1561156_at | 6.67E−04 | — |
| 4331 | 231937_at | 6.67E−04 | — |
| 4331 | 1570100_at | 0.001064 | — |
| 4331 | 241816_at | 0.0012 | — |
| 4331 | 1554704_at | 6.67E−04 | ATP8B3 |
| 4331 | 1558621_at | 8.00E−06 | CABLES1 |
| 4331 | 228516_at | 1.60E−04 | CDAN1 |
| 4331 | 229082_at | 6.00E−07 | CCDC125 |
| 4331 | 238863_x_at | 4.26E−04 | COG8 |
| 4331 | 231831_at | 0.0015 | COX19 |
| 4331 | 217501_at | 0.001667 | CIAO1 |
| 4331 | 233837_at | 2.00E−04 | GGT1 |
| 4331 | 1560227_at | 4.96E−04 | GDPD1 |
| 4331 | 1557621_at | 5.62E−04 | KCP |
| 4331 | 1557197_a_at | 2.01E−04 | LGALS3 |
| 4331 | 205887_x_at | 0.00102 | MSH3 |
| 4331 | 1568974_at | 0.001028 | NHEG1 |
| 4331 | 215857_at | 3.65E−05 | NCLN |
| 4331 | 219418_at | 0.001063 | NHEJ1 /// SLC23A3 |
| 4331 | 239748_x_at | 7.00E−04 | OCIAD1 |
| 4331 | 1563690_at | 5.10E−04 | PPP4R1 |
| 4331 | 1555452_at | 2.04E−04 | RALGPS1 |
| 4331 | 226056_at | 0.001007 | ARHGAP31 |
| 4331 | 1554703_at | 6.00E−07 | ARHGEF10 |
| 4331 | 215588_x_at | 6.67E−04 | RIOK3 |
| 4331 | 208322_s_at | 5.61E−04 | ST3GAL1 |
| 4331 | 215623_x_at | 4.53E−05 | SMC4 |
| 4331 | 216310_at | 2.00E−06 | TAOK1 |
| 4331 | 233174_at | 1.50E−04 | LOC100287015 |
| 4331 | 1570350_at | 5.00E−08 | LOC100506403 /// RUNX1 |
| 4331 | 215709_at | 5.00E−08 | LOC100653079 /// PRIM2 |
| 4331 | 210065_s_at | 7.02E−04 | UPK1B |
| 4331 | 220104_at | 3.84E−05 | ZC3HAV1 |
| 4331 | 1569557_at | 5.04E−04 | ZNF248 |
| 342 | 1553909_x_at | 6.00E−04 | FAM178A |
| 345 | 1559629_at | 2.85E−04 | — |
| 345 | 231883_at | 0.001 | FBXW8 |
| 345 | 216710_x_at | 0.001322 | ZNF287 |
| 4393 | 204062_s_at | 0.004 | ULK2 |
| 347 | 242182_x_at | 2.00E−05 | — |
| 4412 | 1568914_at | 3.00E−05 | OPN5 |
| 4412 | 242617_at | 4.00E−04 | TMED8 |
| 4426 | 233319_x_at | 6.81E−04 | — |
| 4426 | 1560774_at | 7.90E−04 | — |
| 4426 | 1562358_at | 0.001216 | — |
| 4426 | 1558199_at | 0.003 | FN1 |
| 4426 | 217595_at | 0.0026 | GSPT1 |
| 4426 | 206295_at | 0.001502 | IL18 |
| 4426 | 1562338_at | 0.003 | 01-mrt |
| 4426 | 232169_x_at | 9.35E−04 | MIR4691 /// NDUFS8 |
| 4426 | 242625_at | 0.001057 | RSAD2 |
| 4426 | 218155_x_at | 0.001751 | TSR1 |
| 4447 | 242315_at | 4.00E−04 | — |
| 4447 | 231979_at | 0.001336 | — |
| 4447 | 239788_at | 0.004 | — |
| 4447 | 217588_at | 0.001336 | CATSPER2 /// CATSPER2P1 |
| 4447 | 213804_at | 0.004 | INPP5B |
| 4447 | 225073_at | 0.001 | PPHLN1 |
| 4447 | 1558407_at | 7.46E−04 | PLEKHG2 |
| 4447 | 235037_at | 0.001336 | TMEM41A |
| 4447 | 231904_at | 0.00135 | U2AF1 |
| 4447 | 1553145_at | 0.001333 | FLJ39653 |
| 4447 | 1562305_x_at | 0.004 | ZKSCAN3 |
| 4456 | 232505_at | 4.40E−04 | DCTN2 |
| 4456 | 1558727_at | 2.00E−04 | SERP2 |
| 4484 | 1565576_at | 4.00E−06 | — |
| 4484 | 224105_x_at | 1.02E−05 | — |
| 4484 | 234382_x_at | 3.67E−05 | — |
| 4484 | 1563241_at | 2.00E−04 | — |
| 4484 | 233739_at | 0.002 | — |
| 4484 | 222796_at | 3.00E−07 | ATP5J2-PTCD1 /// PTCD1 |
| 4484 | 220588_at | 1.00E−05 | BCAS4 |
| 4484 | 234938_at | 4.00E−06 | BACH1 |
| 4484 | 211464_x_at | 3.00E−07 | CASP6 |
| 4484 | 224712_x_at | 4.00E−06 | C19orf42 |
| 4484 | 207780_at | 4.00E−06 | CYLC2 |
| 4484 | 217133_x_at | 3.00E−07 | CYP2B6 |
| 4484 | 205998_x_at | 4.00E−06 | CYP3A4 |
| 4484 | 222886_at | 3.00E−07 | NSUN3 |
| 4484 | 235767_x_at | 2.10E−06 | PHAX |
| 4484 | 206021_at | 1.00E−06 | SCAND2 |
| 4484 | 1569793_at | 7.53E−05 | SLC25A18 |
| 4484 | 231923_at | 1.00E−05 | TMEM150C |
| 4484 | 231775_at | 3.33E−04 | TNFRSF10A |
| 4511 | 214589_at | 1.25E−04 | FGF12 |
| 4528 | 211781_x_at | 4.55E−04 | — |
| 4549 | 216735_x_at | 8.82E−04 | — |
| 4549 | 1559716_at | 5.10E−04 | INO80C |
| 4557 | 225609_at | 0.001 | GSR |
| 4579 | 222286_at | 0.0035 | SNAPC3 |
| 4583 | 1565837_at | 5.00E−05 | — |
| 4583 | 236765_at | 4.00E−04 | — |
| 4583 | 243895_x_at | 4.00E−04 | — |
| 4583 | 1557712_x_at | 4.50E−04 | — |
| 4583 | 1557710_at | 4.50E−04 | — |
| 4583 | 221155_x_at | 5.86E−04 | — |
| 4583 | 1560395_at | 7.03E−04 | — |
| 4583 | 217531_at | 8.63E−04 | — |
| 4583 | 216811_at | 8.92E−04 | — |
| 4583 | 234643_x_at | 0.002251 | — |
| 4583 | 1554631_at | 1.82E−04 | ATM |
| 4583 | 217141_at | 5.27E−04 | BTBD7 |
| 4583 | 221550_at | 0.001333 | COX15 |
| 4583 | 206755_at | 0.00145 | CYP2B6 |
| 4583 | 1555629_at | 0.001 | HAVCR2 |
| 4583 | 220089_at | 2.85E−04 | L2HGDH |
| 4583 | 216167_at | 4.06E−04 | LRRN2 |
| 4583 | 211768_at | 4.00E−04 | LAT2 |
| 4583 | 217584_at | 8.00E−06 | NPC1 |
| 4583 | 211697_x_at | 3.00E−04 | PNO1 |
| 4583 | 220796_x_at | 5.85E−04 | SLC35E1 |
| 4583 | 231770_x_at | 3.67E−04 | WDPCP |
| 4583 | 232911_at | 2.63E−04 | ZFP14 |
| 4583 | 1554985_at | 0.001334 | ZNF396 |
| 4586 | 231231_at | 2.00E−04 | — |
| 4586 | 228101_at | 0.002 | APBA1 |
| 4586 | 219028_at | 2.00E−04 | HIPK2 |
| 4586 | 231039_at | 0.002 | NAA30 |
| 4586 | 235263_at | 6.00E−04 | STAG3L1 /// STAG3L2 /// STAG3L3 |
| 366 | 1564773_x_at | 5.63E−04 | — |
| 366 | 217569_x_at | 8.72E−04 | — |
| 366 | 211374_x_at | 0.0013 | — |
| 366 | 1553271_at | 0.001133 | DIP2B |
| 366 | 206565_x_at | 7.65E−04 | SMA4 |
| 366 | 219846_at | 7.33E−04 | GON4L |
| 366 | 1569815_x_at | 0.001025 | STRN |
| 366 | 1569813_at | 0.001025 | STRN |
| 366 | 230623_x_at | 0.001001 | USP28 |
| 366 | 1564240_at | 4.80E−04 | LOC100130856 |
| 4620 | 1556224_a_at | 5.00E−20 | LOC155060 /// ZNF783 |
| 4621 | 216261_at | 0.001333 | ITGB3 |
| 4677 | 1569491_at | 8.40E−04 | — |
| 4677 | 1565809_x_at | 0.001002 | — |
| 4677 | 241993_x_at | 0.001703 | — |
| 4677 | 1554314_at | 3.44E−04 | C6orf141 |
| 4677 | 237475_x_at | 7.80E−04 | CCDC152 |
| 4677 | 1556235_at | 3.24E−04 | LOC100506560 /// LOC100509195 |
| 378 | 240906_at | 0.001302 | — |
| 379 | 1570054_at | 9.99E−04 | — |
| 379 | 1566243_at | 0.001007 | — |
| 379 | 1569540_at | 0.00109 | — |
| 379 | 214808_at | 0.001242 | — |
| 379 | 1566787_at | 0.001302 | — |
| 379 | 1557532_at | 0.00147 | — |
| 379 | 1561829_at | 0.001525 | — |
| 379 | 1566848_x_at | 0.002 | — |
| 379 | 244045_at | 0.002005 | — |
| 379 | 235826_at | 0.002005 | — |
| 379 | 229255_x_at | 0.002015 | — |
| 379 | 234182_at | 0.00205 | — |
| 379 | 1570587_at | 0.0022 | — |
| 379 | 241777_x_at | 0.002275 | — |
| 379 | 217662_x_at | 0.002367 | — |
| 379 | 1566518_at | 0.002367 | — |
| 379 | 209646_x_at | 7.74E−04 | ALDH1B1 |
| 379 | 1559982_s_at | 0.002005 | AKR1E2 |
| 379 | 206738_at | 7.94E−04 | APOC2 /// APOC4 /// APOC4-APOC2 |
| 379 | 207328_at | 8.93E−04 | ALOX15 |
| 379 | 201605_x_at | 0.001087 | CNN2 |
| 379 | 230379_x_at | 3.91E−04 | C2orf56 |
| 379 | 1557782_s_at | 2.80E−04 | C21orf49 |
| 379 | 215151_at | 0.001 | DOCK10 |
| 379 | 1558546_at | 0.0018 | DNASE1 |
| 379 | 232603_at | 5.27E−04 | DCDC5 |
| 379 | 233375_at | 0.00222 | EFCAB2 |
| 379 | 1568891_x_at | 0.001 | FANCD2 |
| 379 | 1568889_at | 0.001 | FANCD2 |
| 379 | 233106_at | 0.002005 | FRMD6-AS1 |
| 379 | 223887_at | 0.001493 | GPR132 |
| 379 | 215490_at | 0.002005 | IBA57 |
| 379 | 232756_at | 2.52E−04 | KALRN |
| 379 | 204449_at | 0.001433 | PDCL |
| 379 | 234971_x_at | 8.73E−04 | PLCD3 |
| 379 | 1553136_at | 0.001006 | KCTD18 |
| 379 | 201404_x_at | 2.73E−04 | PSMB2 |
| 379 | 243792_x_at | 0.001 | PTPN13 |
| 379 | 1559529_at | 0.002015 | PTK2 |
| 379 | 1555324_at | 9.94E−04 | PTK7 |
| 379 | 1554800_at | 9.33E−04 | RAB39A |
| 379 | 1559582_at | 0.00137 | RHOQ |
| 379 | 207489_at | 0.001093 | RPS2P45 |
| 379 | 219493_at | 0.001582 | SHCBP1 |
| 379 | 1558331_at | 0.00204 | SIRT2 |
| 379 | 225002_s_at | 0.001018 | SUMF2 |
| 379 | 235651_at | 0.001577 | TTC22 |
| 379 | 239593_at | 0.0016 | TMEM213 |
| 379 | 244730_x_at | 0.00205 | LOC100129195 |
| 379 | 224766_at | 0.001805 | LOC100506548 /// RPL37 |
| 379 | 210917_at | 0.002005 | YES1 |
| 379 | 1557036_at | 0.002005 | ZBTB1 |
| 379 | 1553781_at | 0.002005 | ZC3HAV1L |
| 379 | 233175_at | 0.001488 | ZNF460 |
| 379 | 205881_at | 0.001536 | ZNF74 |
| 379 | 1569277_at | 8.41E−04 | ZNF91 |
| 4737 | 243147_x_at | 5.00E−06 | — |
| 4737 | 242453_at | 2.00E−05 | — |
| 4737 | 243640_x_at | 6.00E−05 | — |
| 4737 | 1563114_at | 6.00E−05 | — |
| 4737 | 1556416_s_at | 6.00E−05 | — |
| 4737 | 1553462_at | 6.00E−05 | — |
| 4737 | 232957_x_at | 1.00E−04 | — |
| 4737 | 1563704_at | 2.00E−04 | — |
| 4737 | 1561959_x_at | 0.001 | — |
| 4737 | 239196_at | 2.55E−05 | ANKRD22 |
| 4737 | 242280_x_at | 3.00E−04 | CPEB4 |
| 4737 | 218498_s_at | 2.00E−05 | ERO1L |
| 4737 | 215942_s_at | 7.33E−04 | GTSE1 |
| 4737 | 234035_at | 0.0025 | HNRNPM |
| 4737 | 244703_x_at | 3.00E−04 | IPO9 |
| 4737 | 235321_at | 5.00E−06 | NDUFS1 |
| 4737 | 224738_x_at | 5.05E−06 | RPL7L1 |
| 4737 | 242364_x_at | 2.00E−05 | LOC100131096 |
| 4737 | 236433_at | 4.00E−07 | LOC100506713 /// MIR195 /// MIR497 /// MIR497HG |
| 4786 | 237504_at | 2.00E−20 | INTS10 |
| 4788 | 208301_at | 9.00E−06 | — |
| 4788 | 1559372_at | 9.00E−06 | — |
| 4788 | 235407_at | 3.00E−05 | — |
| 4788 | 224758_at | 1.00E−04 | C7orf73 |
| 4788 | 214088_s_at | 9.00E−06 | FUT3 |
| 4788 | 213650_at | 9.00E−06 | GOLGA8A /// GOLGA8B /// LOC100508892 |
| 4788 | 1555318_at | 7.00E−07 | HIF3A |
| 4788 | 208200_at | 3.00E−05 | IL1A |
| 4788 | 223817_at | 9.00E−06 | LRRIQ1 |
| 4788 | 238931_at | 7.00E−07 | METTL16 |
| 4788 | 215462_at | 7.00E−07 | PLK3 |
| 4788 | 215849_x_at | 3.00E−06 | TTC18 |
| 4789 | 224958_at | 0.001 | NUFIP2 |
| 4793 | 208144_s_at | 3.76E−04 | — |
| 4803 | 1561663_at | 0.00115 | — |
| 4803 | 207431_s_at | 2.50E−04 | DEGS1 |
| 4808 | 210919_at | 3.88E−04 | PHLPP1 |
| 4811 | 214967_at | 0.002 | — |
| 4811 | 239948_at | 0.002 | NUP153 |
| 4814 | 230606_at | 0.002 | LOC100287525 |
| 4816 | 1566580_at | 0.004 | — |
| 4816 | 207760_s_at | 0.004 | NCOR2 |
| 4816 | 213917_at | 0.0023 | PAX8 |
| 4865 | 207175_at | 1.67E−05 | ADIPOQ |
| 4865 | 1566150_at | 4.33E−07 | CALML4 |
| 4865 | 223248_at | 2.69E−04 | HSDL1 |
| 4883 | 1554480_a_at | 3.37E−04 | ARMC10 |
| 4883 | 202714_s_at | 0.003 | KIAA0391 |
| 4883 | 219007_at | 0.003 | NUP43 |
| 4883 | 1558712_at | 5.82E−04 | TMEM134 |
| 4883 | 238772_at | 0.003 | ZNF207 |
| 4889 | 214983_at | 0.004 | TTTY15 |
| 4906 | 215484_at | 8.00E−05 | — |
| 4906 | 1560755_at | 8.00E−05 | — |
| 4906 | 1559259_at | 8.00E−05 | — |
| 4906 | 1563204_at | 2.35E−04 | — |
| 4906 | 238286_at | 3.00E−04 | — |
| 4906 | 243537_at | 3.40E−04 | — |
| 4906 | 1561240_at | 0.001 | — |
| 4906 | 1557398_at | 0.002333 | — |
| 4906 | 231111_at | 0.003 | — |
| 4906 | 1554618_at | 8.00E−05 | AGFG2 |
| 4906 | 235459_at | 0.003 | RNF41 |
| 4906 | 224139_at | 7.71E−04 | SHANK2-AS3 |
| 4906 | 203508_at | 0.001 | TNFRSF1B |
| 4906 | 1553258_at | 8.00E−05 | FLJ30679 |
| 4906 | 243161_x_at | 3.00E−04 | ZFP42 |
| 4907 | 213791_at | 4.00E−12 | PENK |
| 4946 | 225884_s_at | 0.005 | GZF1 |
| 4950 | 233152_x_at | 1.00E−04 | — |
| 4972 | 205193_at | 7.47E−04 | MAFF |
| 4973 | 235127_at | 1.00E−13 | PMP2 |
| 5016 | 231992_x_at | 6.72E−04 | — |
| 5043 | 1560499_at | 1.69E−04 | C14orf64 |
| 415 | 216114_at | 0.004 | NCKIPSD |
| 416 | 233757_x_at | 3.24E−04 | — |
| 5114 | 1565740_at | 1.00E−05 | — |
| 5114 | 211050_x_at | 7.64E−04 | LOC100134822 /// LOC100288069 /// LOC100653347 |
| 5114 | 1569108_a_at | 0.001 | ZNF589 |
| 5117 | 1560696_x_at | 6.77E−04 | QTRTD1 |
| 5117 | 1560695_at | 6.77E−04 | QTRTD1 |
| 5149 | 234346_x_at | 3.50E−06 | — |
| 5149 | 206296_x_at | 5.51E−05 | MAP4K1 |
| 5149 | 1569856_at | 2.41E−04 | TPP2 |
| 5212 | 1562010_x_at | 0.003201 | — |
| 5212 | 1559452_a_at | 0.003331 | — |
| 5212 | 241593_x_at | 0.003624 | — |
| 5212 | 1569345_at | 0.003715 | — |
| 5212 | 1569341_at | 0.004101 | — |
| 5212 | 242266_x_at | 0.004218 | — |
| 5212 | 240988_x_at | 0.004283 | — |
| 5212 | 233805_at | 0.004582 | — |
| 5212 | 1562313_at | 0.003193 | BCORP1 |
| 5212 | 1569554_at | 0.002346 | ESR2 |
| 5212 | 211740_at | 0.004 | ICA1 |
| 5212 | 238600_at | 0.003665 | JAKMIP1 |
| 5212 | 224673_at | 0.004 | LENG8 |
| 5212 | 1554589_at | 0.004 | LENG9 |
| 5212 | 244338_at | 0.003665 | LDLRAD3 |
| 5212 | 216436_at | 0.003421 | PIK3R4 |
| 5212 | 1554710_at | 0.00265 | KCNMB1 |
| 5212 | 235964_x_at | 0.003784 | SAMHD1 |
| 5212 | 235529_x_at | 0.003977 | SAMHD1 |
| 5212 | 219620_x_at | 0.002974 | TOR4A |
| 5212 | 233589_x_at | 0.00335 | TOR4A |
| 5212 | 222976_s_at | 0.004333 | TPM3 |
| 5212 | 238901_at | 0.00454 | LOC100505633 |
| 5212 | 1561442_at | 0.004309 | LOC283585 |
| 5212 | 1563978_at | 0.002833 | LOC728690 |
| 5212 | 203604_at | 0.005 | ZNF516 |
| 5216 | 208185_x_at | 0.001018 | — |
| 5314 | 1556678_a_at | 2.10E−04 | — |
| 5316 | 1560798_at | 0.001528 | — |
| 5320 | 220732_at | 0.001025 | PREX2 |
| 5328 | 1560543_at | 0.001334 | GRK4 |
| 5336 | 243189_at | 1.00E−05 | NRF1 |
| 5345 | 244891_x_at | 1.50E−05 | — |
| 5345 | 1555205_at | 0.001333 | — |
| 5345 | 217253_at | 7.52E−04 | SH3BP2 |
| 5360 | 244146_at | 0.003 | DTWD1 |
| 5381 | 1560662_s_at | 4.80E−05 | WHAMMP2 /// WHAMMP3 |
| 5382 | 200775_s_at | 1.00E−12 | HNRNPK /// MIR7-1 |
| 439 | 221251_x_at | 0.005 | INO80B /// INO80B-WBP1 |
| 5409 | 236628_at | 1.70E−04 | — |
| 5409 | 232997_at | 0.001001 | — |
| 5409 | 1562236_at | 1.80E−04 | KAT6B |
| 5409 | 216509_x_at | 0.002 | MLLT10 |
| 5409 | 216506_x_at | 0.002 | MLLT10 |
| 5409 | 1563051_at | 0.00101 | OSBP |
| 5409 | 226120_at | 5.00E−04 | TTC8 |
| 5411 | 214594_x_at | 7.93E−04 | ATP8B1 |
| 5416 | 234773_x_at | 5.33E−05 | — |
| 5416 | 215965_at | 2.00E−04 | — |
| 5416 | 217105_at | 7.50E−04 | — |
| 5416 | 217102_at | 7.50E−04 | — |
| 5416 | 217446_x_at | 9.00E−04 | — |
| 5416 | 235650_at | 1.01E−04 | CDHR3 /// LOC100652903 |
| 5416 | 221064_s_at | 6.00E−04 | UNKL |
| 5430 | 232896_at | 8.00E−05 | ERBB21P |
| 5502 | 212373_at | 5.00E−04 | FEM1B |
| 5523 | 217682_at | 1.65E−04 | — |
| 5526 | 234789_at | 5.50E−04 | — |
| 5526 | 224605_at | 6.00E−04 | C4orf3 |
| 5526 | 232768_at | 2.00E−04 | CCNB2 |
| 5526 | 237866_at | 6.00E−04 | PID1 |
| 5526 | 1569446_x_at | 0.002 | LOC100287576 /// LOC100506454 |
| 5526 | 228346_at | 4.50E−04 | ZNF844 |
| 5543 | 231048_at | 0.003 | — |
| 5543 | 233132_at | 0.003 | LOC96610 |
| 9457 | 1570244_at | 2.00E−06 | — |
| 9457 | 1570476_at | 6.00E−06 | — |
| 9457 | 233407_at | 6.67E−05 | — |
| 9457 | 1561662_at | 2.33E−04 | — |
| 9457 | 1570335_at | 2.37E−04 | — |
| 9457 | 242646_at | 2.47E−04 | — |
| 9457 | 242476_at | 3.00E−04 | — |
| 9457 | 240535_at | 6.68E−04 | — |
| 9457 | 217443_at | 6.68E−04 | — |
| 9457 | 244788_at | 0.001 | — |
| 9457 | 232544_at | 0.002001 | — |
| 9457 | 225065_x_at | 8.00E−05 | C17orf76-AS1 /// SNORD49A /// SNORD49B /// SNORD65 |
| 9457 | 201633_s_at | 3.00E−04 | CYB5B |
| 9457 | 201789_at | 0.003 | DHRS7 |
| 9457 | 233089_at | 2.40E−05 | QRSL1 |
| 9457 | 204544_at | 0.001 | HPS5 |
| 9457 | 211732_x_at | 6.67E−04 | HNMT |
| 9457 | 232844_at | 2.00E−06 | IFT140 |
| 9457 | 242778_at | 0.001 | LPXN |
| 9457 | 1562142_at | 2.34E−04 | SKIV2L2 |
| 9457 | 226662_at | 2.35E−04 | STX17 |
| 9457 | 201746_at | 2.87E−04 | TP53 |
| 9457 | 1554777_at | 0.001 | ZFP42 |
| 9460 | 211590_x_at | 1.00E−09 | TBXA2R |
| 471 | 206907_at | 0.002 | TNFSF9 |
| 9463 | 239227_at | 3.00E−04 | — |
| 9463 | 1569311_at | 3.00E−04 | — |
| 9463 | 1565846_at | 3.00E−04 | — |
| 9463 | 1557155_a_at | 3.00E−04 | — |
| 9463 | 239649_at | 0.001 | — |
| 9463 | 1560346_at | 0.001 | — |
| 9463 | 239744_at | 0.004 | — |
| 9463 | 244748_at | 0.004 | — |
| 9463 | 243620_at | 0.004 | — |
| 9463 | 242747_at | 0.004 | — |
| 9463 | 240791_at | 0.004 | — |
| 9463 | 240541_at | 0.004 | — |
| 9463 | 237398_at | 0.004 | — |
| 9463 | 1562084_at | 0.004 | — |
| 9463 | 203741_s_at | 9.00E−05 | ADCY7 |
| 9463 | 222608_s_at | 3.00E−04 | ANLN |
| 9463 | 216120_s_at | 0.004 | ATP2B2 |
| 9463 | 242197_x_at | 3.00E−04 | CD36 |
| 9463 | 211173_at | 0.004 | CCKAR |
| 9463 | 218182_s_at | 0.004 | CLDN1 |
| 9463 | 205223_at | 3.00E−04 | DEPDC5 |
| 9463 | 230869_at | 0.004 | FAM155A |
| 9463 | 209841_s_at | 0.004 | LRRN3 |
| 9463 | 203062_s_at | 0.004 | MDC1 |
| 9463 | 215880_at | 0.004 | NAGLU |
| 9463 | 204621_s_at | 0.004 | NR4A2 |
| 9463 | 224892_at | 3.00E−04 | PLDN |
| 9463 | 216254_at | 3.00E−04 | PARVB |
| 9463 | 1568764_x_at | 3.00E−04 | LOC728613 /// PDCD6 |
| 9463 | 1568762_at | 3.00E−04 | LOC728613 /// PDCD6 |
| 9463 | 231851_at | 0.001 | RAVER2 |
| 9463 | 238430_x_at | 3.00E−04 | SLFN5 |
| 9463 | 207570_at | 0.004 | SHOX |
| 9463 | 214925_s_at | 0.004 | SPTAN1 |
| 9463 | 227205_at | 0.001 | TAF1 |
| 9463 | 1556154_a_at | 0.004 | MGC23284 |
| 9463 | 1564360_a_at | 0.002 | LOC339260 |
| 9463 | 1559276_at | 3.00E−04 | LOC728606 |
| 9463 | 201244_s_at | 9.00E−05 | RAF1 |
| 9475 | 217533_x_at | 1.00E−04 | — |
| 9475 | 1566725_at | 9.74E−04 | — |
| 9501 | 233390_at | 3.00E−04 | — |
| 9501 | 1562145_at | 5.00E−04 | — |
| 9501 | 211164_at | 4.70E−05 | EPHA3 |
| 9512 | 210259_s_at | 0.004 | DLX4 |
| 9515 | 217713_x_at | 2.14E−04 | — |
| 9515 | 207598_x_at | 3.00E−05 | XRCC2 |
| 9556 | 215615_x_at | 1.00E−05 | — |
| 9556 | 1563329_s_at | 0.001122 | — |
| 9556 | 1566798_at | 0.001333 | — |
| 9556 | 221540_x_at | 4.50E−06 | GTF2H2 /// GTF2H2B /// GTF2H2C /// GTF2H2D |
| 9556 | 207092_at | 0.001002 | LEP |
| 9556 | 232190_x_at | 2.52E−04 | LOC100133445 /// LOC115110 |
| 9573 | 1563147_at | 0.001002 | — |
| 9573 | 216166_at | 0.001075 | — |
| 9573 | 206936_x_at | 0.001456 | — |
| 9573 | 1570361_a_at | 0.001673 | — |
| 9573 | 222517_at | 5.64E−04 | AP3M1 |
| 9573 | 214707_x_at | 7.57E−04 | ALMS1 |
| 9573 | 218056_at | 0.001312 | BFAR |
| 9573 | 234094_x_at | 0.00139 | DNAJC21 |
| 9573 | 220838_at | 9.73E−04 | EXD3 |
| 9573 | 205973_at | 6.22E−04 | FEZ1 |
| 9573 | 222187_x_at | 8.00E−04 | G3EP1 |
| 9573 | 1555685_at | 6.38E−04 | H6PD |
| 9573 | 222521_x_at | 0.001043 | NDUFC2 /// NDUFC2-KCTD14 |
| 9573 | 205636_at | 0.001 | SH3GL3 |
| 483 | 217152_at | 3.00E−04 | — |
| 483 | 1559566_at | 4.36E−04 | FBXO42 |
| 483 | 216115_at | 0.0019 | NF1 |
| 9597 | 1560332_at | 0.004 | — |
| 9598 | 233806_at | 2.00E−04 | — |
| 9602 | 1566162_x_at | 5.00E−04 | — |
| 9602 | 1566161_at | 5.00E−04 | — |
| 9602 | 220847_x_at | 2.50E−04 | ZNF221 |
| 9628 | 236240_at | 0.001022 | C4orf29 |
| 9630 | 1566527_at | 2.00E−06 | — |
| 9630 | 1570272_at | 7.00E−06 | — |
| 9630 | 243203_at | 2.15E−04 | — |
| 9630 | 1562416_at | 3.34E−04 | — |
| 9630 | 233904_at | 3.50E−04 | — |
| 9630 | 238743_at | 3.90E−04 | — |
| 9630 | 1570165_at | 8.00E−04 | — |
| 9630 | 239251_at | 0.001 | — |
| 9630 | 242347_at | 0.001234 | — |
| 9630 | 1555114_at | 5.00E−04 | ATP6V0A2 |
| 9630 | 213020_at | 0.001567 | GOSR1 |
| 9630 | 236906_x_at | 3.50E−04 | IPO5 |
| 9630 | 206341_at | 3.50E−04 | IL2RA |
| 9630 | 1560558_at | 2.15E−04 | INIP |
| 9630 | 232921_at | 2.00E−05 | KIAA1549 |
| 9630 | 215063_x_at | 3.50E−04 | LRRC40 |
| 9630 | 231876_at | 1.75E−04 | TRIM56 |
| 9630 | 1553335_x_at | 0.001233 | LOC285696 |
| 9630 | 1563637_at | 8.00E−06 | LOC729652 |
| 9647 | 238121_at | 0.003 | GK5 |
| 492 | 232811_x_at | 5.26E−04 | PRICKLE1 |
| 9707 | 209109_s_at | 5.00E−21 | TSPAN6 |
| 9718 | 1570412_at | 0.001 | — |
| 9740 | 220339_s_at | 0.003 | TPSG1 |
| 9750 | 233412_x_at | 0.002039 | — |
| 9780 | 1560033_at | 5.00E−04 | MCCC2 |
| 504 | 204713_s_at | 1.00E−07 | F5 |
| 505 | 209913_x_at | 0.001506 | AP5Z1 |
| 9814 | 1566862_at | 0.002 | — |
| 9816 | 241376_at | 0.003 | — |
| 9831 | 221152_at | 6.00E−07 | COL8A1 |
| 9840 | 234394_at | 4.00E−08 | ZNF124 |
| 9924 | 1554503_a_at | 3.51E−04 | OSCAR |
| 521 | 200845_s_at | 1.00E−07 | PRDX6 |

^{A}shows the SEQ ID NO;
^{B}Affymetrix probe ID that harbors significant identity with the spacers from (A);
^{C}shows the significance of the identity;
^{D}shows the transcript that the Affymetrix probe detects

For the human CRISPR repeats this number exceeded 3000 (Table 7).

TABLE 7
Affymetrix probes harbor significant identity with the human
CRISPR repeats.

| SEQ | | | |
|---|---|---|---|
| ID NO^{A} | Probe Set ID^{B} | E-Value^{C} | Gene Target Symbol^{D} |
| 542 | 1566202_at | 5.00E−07 | — |
| 542 | 232712_at | 1.33E−04 | — |
| 559 | 240025_x_at | 8.01E−04 | — |
| 559 | 242453_at | 9.64E−04 | — |
| 559 | 240804_at | 5.59E−04 | — |
| 559 | 1566096_x_at | 2.10E−04 | — |
| 559 | 1561205_at | 2.60E−05 | — |
| 559 | 1559530_at | 2.83E−04 | — |
| 559 | 243310_at | 2.00E−04 | — |
| 559 | 244045_at | 2.06E−04 | — |
| 559 | 207479_at | 1.92E−04 | — |
| 559 | 1560297_at | 5.73E−04 | — |
| 559 | 216773_at | 0.0023 | — |
| 559 | 243537_at | 0.00166 | — |
| 559 | 241993_x_at | 6.80E−04 | — |
| 559 | 241737_x_at | 1.34E−04 | — |
| 559 | 233519_at | 1.99E−04 | — |
| 559 | 1570335_at | 5.06E−04 | — |
| 559 | 1566503_at | 1.65E−04 | — |
| 559 | 1566424_at | 2.53E−04 | — |
| 559 | 1560755_at | 1.58E−04 | — |
| 559 | 241686_x_at | 9.51E−04 | — |
| 559 | 233875_at | 2.19E−04 | — |
| 559 | 1559629_at | 5.02E−04 | — |
| 559 | 242990_at | 0.00168 | — |
| 560 | 1560199_x_at | 3.00E−06 | — |
| 573 | 217137_x_at | 4.80E−06 | — |
| 573 | 233108_at | 7.67E−06 | — |
| 619 | 241391_at | 0.003 | — |
| 663 | 1563204_at | 1.28E−04 | — |
| 663 | 231111_at | 0.00101 | — |
| 663 | 1557398_at | 0.00134 | — |
| 663 | 1561240_at | 5.40E−04 | — |
| 663 | 232493_at | 0.001 | — |
| 663 | 233981_at | 0.001 | — |
| 663 | 1570151_at | 0.001 | — |
| 663 | 237600_at | 8.00E−05 | — |
| 663 | 234505_at | 0.003 | — |
| 716 | 243646_at | 0.001 | — |
| 720 | 242310_at | 1.00E−06 | — |
| 738 | 239815_at | 0.001367 | — |
| 762 | 1563480_at | 2.00E−06 | — |
| 790 | 244714_at | 6.70E−04 | — |
| 790 | 233473_x_at | 3.00E−05 | — |
| 790 | 1561956_at | 6.70E−06 | — |
| 790 | 1561606_at | 6.53E−04 | — |
| 790 | 231674_at | 1.00E−04 | — |
| 796 | 216290_x_at | 1.98E−04 | — |
| 796 | 216163_at | 8.86E−05 | — |
| 796 | 235207_at | 1.35E−04 | — |
| 796 | 231979_at | 6.06E−05 | — |
| 796 | 227509_x_at | 1.61E−04 | — |
| 796 | 1569732_at | 3.00E−05 | — |
| 796 | 1566541_at | 8.73E−06 | — |
| 796 | 1566485_at | 5.60E−05 | — |
| 796 | 1562010_x_at | 8.55E−05 | — |
| 796 | 1560576_at | 1.62E−05 | — |
| 796 | 1558275_at | 5.23E−04 | — |
| 796 | 1557581_x_at | 8.92E−05 | — |
| 796 | 1557580_at | 3.01E−05 | — |
| 796 | 233419_at | 2.33E−04 | — |
| 796 | 232872_at | 2.33E−06 | — |
| 796 | 232468_at | 1.19E−04 | — |
| 796 | 237402_at | 3.97E−06 | — |
| 796 | 215360_at | 3.44E−05 | — |
| 796 | 243846_x_at | 1.04E−06 | — |
| 796 | 211374_x_at | 4.29E−05 | — |
| 796 | 241223_x_at | 3.74E−04 | — |
| 796 | 216756_at | 1.65E−06 | — |
| 796 | 1561597_x_at | 7.58E−05 | — |
| 796 | 1561596_at | 1.87E−05 | — |
| 796 | 1563721_at | 4.92E−04 | — |
| 796 | 238812_at | 2.37E−05 | — |
| 796 | 1559724_at | 5.82E−04 | — |
| 808 | 214019_at | 7.63E−04 | — |
| 5679 | 241593_x_at | 8.12E−04 | — |
| 6071 | 1558714_at | 3.00E−04 | — |
| 6113 | 1566425_at | 1.00E−06 | — |
| 6173 | 1566162_x_at | 0.002001 | — |
| 6173 | 1566161_at | 0.002001 | — |
| 6184 | 1558489_at | 0.0013 | — |
| 6217 | 1570579_at | 4.00E−06 | — |
| 6217 | 1556250_at | 5.17E−06 | — |
| 6217 | 1570099_at | 2.00E−04 | — |
| 6249 | 1556658_a_at | 2.58E−04 | — |
| 6249 | 237768_x_at | 4.61E−04 | — |
| 6249 | 216767_at | 2.72E−04 | — |
| 6249 | 236839_at | 3.78E−04 | — |
| 6249 | 1563147_at | 4.79E−04 | — |
| 6249 | 1554266_at | 7.88E−04 | — |
| 6249 | 215995_x_at | 3.10E−04 | — |
| 6249 | 241888_at | 0.001955 | — |
| 6249 | 217482_at | 9.56E−04 | — |
| 6249 | 238363_at | 0.001254 | — |
| 6249 | 216151_at | 9.63E−04 | — |
| 6249 | 1569459_a_at | 0.0019 | — |
| 6249 | 1561653_at | 0.002333 | — |
| 6249 | 1559895_x_at | 0.001007 | — |
| 6251 | 1555205_at | 1.30E−04 | — |
| 6251 | 1570308_at | 3.91E−04 | — |
| 6251 | 1566798_at | 4.80E−06 | — |
| 6251 | 1562673_at | 2.11E−04 | — |
| 6251 | 1560563_at | 9.34E−05 | — |
| 6251 | 1554599_x_at | 3.72E−05 | — |
| 6251 | 232754_at | 5.39E−05 | — |
| 6251 | 207470_at | 3.20E−04 | — |
| 6251 | 1565976_at | 1.84E−04 | — |
| 6251 | 215628_x_at | 1.89E−04 | — |
| 6251 | 242347_at | 1.57E−04 | — |
| 6251 | 232338_at | 2.10E−04 | — |
| 6251 | 1559229_at | 4.31E−05 | — |
| 6251 | 220583_at | 1.64E−04 | — |
| 6251 | 233783_at | 2.53E−05 | — |
| 5730 | 234019_at | 6.00E−04 | — |
| 5731 | 242675_x_at | 0.002 | — |
| 5731 | 1569805_at | 0.00101 | — |
| 5731 | 1560082_at | 8.35E−04 | — |
| 5731 | 215484_at | 0.002 | — |
| 5732 | 232991_at | 1.16E−04 | — |
| 5732 | 1569784_at | 3.08E−04 | — |
| 5732 | 1568892_at | 2.92E−04 | — |
| 5733 | 234201_x_at | 0.001358 | — |
| 6305 | 224549_x_at | 1.37E−07 | — |
| 6310 | 235274_at | 1.98E−04 | — |
| 6310 | 1556678_a_at | 0.001178 | — |
| 6310 | 243216_x_at | 0.00115 | — |
| 6310 | 1562677_at | 0.002 | — |
| 5736 | 216793_x_at | 4.07E−04 | — |
| 5736 | 216229_x_at | 1.30E−04 | — |
| 5736 | 234266_at | 4.01E−04 | — |
| 5736 | 233440_at | 9.49E−05 | — |
| 5736 | 211411_at | 1.32E−04 | — |
| 5736 | 1570432_at | 5.63E−05 | — |
| 5736 | 1566597_at | 6.34E−05 | — |
| 5736 | 1559682_at | 6.31E−04 | — |
| 5736 | 1569948_at | 1.33E−04 | — |
| 5736 | 1556339_a_at | 5.14E−05 | — |
| 5736 | 221071_at | 4.46E−04 | — |
| 5736 | 216179_x_at | 2.20E−04 | — |
| 5736 | 233319_x_at | 2.52E−04 | — |
| 5736 | 232580_x_at | 2.34E−04 | — |
| 5736 | 1565821_at | 3.06E−04 | — |
| 5736 | 1565820_x_at | 3.06E−04 | — |
| 5736 | 1565819_at | 3.06E−04 | — |
| 5736 | 1559508_at | 8.23E−05 | — |
| 5736 | 216187_x_at | 2.51E−04 | — |
| 5736 | 217446_x_at | 3.34E−04 | — |
| 5736 | 241724_x_at | 2.04E−04 | — |
| 5736 | 217537_x_at | 1.05E−04 | — |
| 5736 | 215868_x_at | 1.39E−04 | — |
| 5736 | 215753_at | 4.46E−04 | — |
| 5736 | 215445_x_at | 2.61E−04 | — |
| 5736 | 234190_at | 1.74E−04 | — |
| 5736 | 233431_x_at | 4.89E−05 | — |
| 5736 | 208246_x_at | 2.97E−04 | — |
| 5736 | 1570054_at | 2.70E−04 | — |
| 5736 | 1566696_at | 1.25E−04 | — |
| 5736 | 1566538_at | 5.19E−05 | — |
| 5736 | 1564449_at | 2.83E−04 | — |
| 5736 | 1561139_at | 2.70E−04 | — |
| 5736 | 1557774_at | 1.25E−04 | — |
| 5736 | 217571_at | 2.27E−04 | — |
| 5736 | 217708_x_at | 4.73E−04 | — |
| 5736 | 216735_x_at | 1.87E−04 | — |
| 5736 | 220689_at | 5.55E−04 | — |
| 5736 | 217703_x_at | 1.41E−04 | — |
| 5736 | 1569231_x_at | 2.13E−04 | — |
| 5736 | 1569230_at | 2.13E−04 | — |
| 5736 | 215907_at | 3.82E−04 | — |
| 6358 | 232670_at | 6.88E−06 | — |
| 6358 | 224098_at | 1.33E−06 | — |
| 6375 | 1558402_at | 0.001423 | — |
| 6385 | 1561234_at | 0.001456 | — |
| 6398 | 224183_at | 5.15E−05 | — |
| 6440 | 212498_at | 0.002 | — |
| 6442 | 234758_at | 0.002 | — |
| 6456 | 237676_at | 0.00185 | — |
| 6456 | 1561341_at | 0.003 | — |
| 5752 | 237458_at | 2.00E−04 | — |
| 6493 | 234582_at | 0.003 | — |
| 6508 | 1566957_at | 5.73E−05 | — |
| 6515 | 1569182_at | 1.82E−04 | — |
| 6520 | 1569999_at | 6.52E−04 | — |
| 6568 | 224114_at | 0.002 | — |
| 6610 | 1556814_a_at | 4.00E−07 | — |
| 6619 | 202015_x_at | 4.00E−07 | — |
| 6619 | 1553882_at | 4.00E−07 | — |
| 6634 | 1566183_at | 4.33E−04 | — |
| 6647 | 1564430_at | 0.00125 | — |
| 6659 | 244866_at | 0.002 | — |
| 6673 | 1557751_at | 0.001067 | — |
| 6673 | 1559436_x_at | 4.00E−05 | — |
| 6675 | 1570165_at | 3.60E−04 | — |
| 6675 | 1565567_at | 4.00E−05 | — |
| 6675 | 1565566_a_at | 4.00E−05 | — |
| 6675 | 238826_x_at | 0.00125 | — |
| 6675 | 233291_at | 7.00E−05 | — |
| 6690 | 229212_at | 4.00E−05 | — |
| 6740 | 243350_at | 6.00E−04 | — |
| 6740 | 1569615_at | 0.002 | — |
| 6772 | 234675_x_at | 3.67E−05 | — |
| 6772 | 220837_at | 0.00125 | — |
| 6811 | 1556327_a_at | 5.62E−06 | — |
| 6811 | 216155_at | 3.11E−05 | — |
| 6811 | 1562962_at | 2.76E−05 | — |
| 6811 | 1562853_x_at | 2.45E−04 | — |
| 6811 | 1562852_at | 2.45E−04 | — |
| 6811 | 1570103_at | 3.53E−05 | — |
| 6811 | 1569542_at | 4.48E−04 | — |
| 6811 | 1560129_at | 3.29E−04 | — |
| 6844 | 215397_x_at | 1.18E−04 | — |
| 6846 | 242795_at | 1.80E−04 | — |
| 6846 | 1568983_a_at | 0.001 | — |
| 6860 | 237187_at | 1.00E−08 | — |
| 5797 | 233658_at | 1.00E−12 | — |
| 6914 | 1564984_at | 1.00E−07 | — |
| 6922 | 242126_at | 1.00E−04 | — |
| 6953 | 234677_at | 9.00E−08 | — |
| 6953 | 1563061_at | 9.00E−08 | — |
| 6953 | 238645_at | 3.50E−04 | — |
| 7009 | 233310_at | 1.18E−04 | — |
| 7009 | 1565857_at | 5.33E−06 | — |
| 7009 | 1564212_at | 3.00E−06 | — |
| 7009 | 220916_at | 4.00E−05 | — |
| 7009 | 1561625_at | 4.00E−05 | — |
| 7009 | 1565621_at | 5.00E−04 | — |
| 7021 | 238881_at | 5.00E−04 | — |
| 7078 | 1570635_at | 5.04E−15 | — |
| 7103 | 222271_at | 0.002 | — |
| 7152 | 234779_at | 4.27E−08 | — |
| 7152 | 234755_x_at | 4.27E−08 | — |
| 5822 | 242315_at | 2.50E−04 | — |
| 5822 | 1567045_at | 6.00E−04 | — |
| 7216 | 1569738_at | 0.003 | — |
| 7267 | 217698_at | 2.77E−04 | — |
| 7268 | 1560511_at | 0.0015 | — |
| 5873 | 1562384_at | 2.83E−04 | — |
| 5873 | 1569474_at | 0.001073 | — |
| 5873 | 244010_at | 8.50E−06 | — |
| 5873 | 224119_at | 3.30E−05 | — |
| 5873 | 1566043_at | 1.19E−04 | — |
| 5873 | 1559843_s_at | 2.64E−06 | — |
| 5873 | 232144_at | 3.48E−04 | — |
| 5873 | 1562752_at | 3.10E−05 | — |
| 5873 | 216337_at | 4.36E−05 | — |
| 7313 | 242371_x_at | 0.002013 | — |
| 7313 | 1570476_at | 5.14E−04 | — |
| 7313 | 1559434_at | 4.29E−04 | — |
| 7313 | 1566787_at | 5.46E−04 | — |
| 7331 | 1560395_at | 0.001015 | — |
| 7331 | 236566_at | 7.29E−04 | — |
| 7331 | 216527_at | 5.52E−04 | — |
| 7331 | 1561959_x_at | 0.001021 | — |
| 7349 | 240695_at | 1.00E−04 | — |
| 7365 | 236072_at | 0.005 | — |
| 7397 | 1563277_at | 0.003 | — |
| 7421 | 234596_at | 4.30E−04 | — |
| 7421 | 216118_at | 3.82E−04 | — |
| 7421 | 1563026_at | 3.15E−04 | — |
| 7421 | 1558208_at | 1.35E−04 | — |
| 7421 | 1566481_at | 4.23E−04 | — |
| 7421 | 1561033_at | 3.19E−04 | — |
| 5851 | 1570630_at | 1.00E−05 | — |
| 7426 | 243246_at | 5.00E−21 | — |
| 7429 | 1563187_at | 0.00185 | — |
| 7472 | 1569345_at | 0.0014 | — |
| 7484 | 1556903_at | 0.002 | — |
| 5610 | 233806_at | 1.55E−04 | — |
| 5610 | 1561749_at | 7.18E−04 | — |
| 5858 | 232877_at | 0.001 | — |
| 7501 | 207750_at | 0.002 | — |
| 7564 | 242080_at | 5.10E−04 | — |
| 7564 | 242805_at | 2.10E−04 | — |
| 7564 | 216169_at | 3.00E−04 | — |
| 7565 | 242731_x_at | 5.90E−04 | — |
| 7566 | 205800_at | 0.002 | — |
| 7567 | 1565788_at | 2.89E−04 | — |
| 7567 | 215965_at | 1.88E−04 | — |
| 7567 | 217591_at | 4.71E−04 | — |
| 5865 | 1566727_at | 0.003 | — |
| 5865 | 1564007_at | 0.003 | — |
| 7577 | 216006_at | 8.00E−06 | — |
| 7577 | 242374_at | 5.00E−04 | — |
| 10116 | 242202_at | 3.00E−07 | — |
| 10019 | 240698_s_at | 3.38E−04 | — |
| 10019 | 239790_s_at | 3.38E−04 | — |
| 10019 | 215862_at | 2.45E−04 | — |
| 10019 | 1565596_at | 8.10E−04 | — |
| 10019 | 1564155_x_at | 3.60E−04 | — |
| 10019 | 1564154_at | 3.60E−04 | — |
| 10019 | 1561704_at | 4.61E−05 | — |
| 10019 | 239817_at | 1.00E−05 | — |
| 10019 | 217662_x_at | 5.26E−05 | — |
| 10019 | 240906_at | 1.85E−04 | — |
| 10019 | 235826_at | 6.36E−04 | — |
| 10019 | 1557532_at | 5.86E−04 | — |
| 10019 | 1570587_at | 0.0014 | — |
| 10019 | 1563210_at | 4.50E−04 | — |
| 10019 | 240160_x_at | 0.0013 | — |
| 5969 | 1561400_at | 6.80E−06 | — |
| 5969 | 1565896_at | 7.00E−04 | — |
| 5969 | 217425_at | 0.00185 | — |
| 7645 | 233596_at | 1.24E−04 | — |
| 7645 | 1561104_at | 3.33E−04 | — |
| 7645 | 238918_at | 6.50E−04 | — |
| 7645 | 1560118_at | 8.80E−04 | — |
| 7649 | 1564763_at | 1.00E−05 | — |
| 7671 | 1569892_at | 6.00E−06 | — |
| 7675 | 207751_at | 1.00E−04 | — |
| 7699 | 241508_at | 6.00E−04 | — |
| 7771 | 211781_x_at | 2.99E−04 | — |
| 7815 | 241777_x_at | 1.33E−04 | — |
| 7815 | 243763_x_at | 4.67E−05 | — |
| 7815 | 242741_x_at | 6.00E−05 | — |
| 7815 | 239892_at | 3.00E−06 | — |
| 7815 | 223872_at | 4.00E−05 | — |
| 5891 | 243682_at | 5.00E−06 | — |
| 5891 | 239804_at | 2.00E−04 | — |
| 5891 | 238286_at | 1.04E−04 | — |
| 5891 | 1569841_x_at | 0.001825 | — |
| 5891 | 1569840_at | 0.00165 | — |
| 5895 | 1559932_at | 1.20E−05 | — |
| 7944 | 235803_at | 0.00152 | — |
| 7944 | 216165_at | 5.50E−04 | — |
| 7964 | 1563460_at | 1.00E−05 | — |
| 7964 | 234531_at | 0.00252 | — |
| 7964 | 1556749_at | 5.00E−04 | — |
| 7980 | 1560557_at | 5.00E−04 | — |
| 7980 | 232757_at | 0.002 | — |
| 7984 | 215504_x_at | 4.00E−05 | — |
| 7984 | 216146_at | 2.18E−05 | — |
| 7984 | 215674_at | 1.08E−04 | — |
| 7984 | 235393_at | 8.33E−05 | — |
| 7984 | 1561662_at | 5.00E−04 | — |
| 7984 | 1570290_at | 5.13E−04 | — |
| 7991 | 239235_at | 0.001313 | — |
| 7992 | 222214_at | 2.08E−04 | — |
| 7992 | 243249_at | 2.90E−04 | — |
| 7992 | 217619_x_at | 3.01E−04 | — |
| 7992 | 1565825_at | 0.00144 | — |
| 7992 | 232029_at | 0.001231 | — |
| 7992 | 1566778_at | 0.00272 | — |
| 7992 | 208154_at | 0.001252 | — |
| 7992 | 217569_x_at | 6.52E−04 | — |
| 7992 | 1566609_at | 9.03E−04 | — |
| 8002 | 237849_at | 1.00E−05 | — |
| 8010 | 232540_x_at | 7.33E−04 | — |
| 8015 | 232800_at | 5.33E−04 | — |
| 8015 | 215861_at | 7.68E−04 | — |
| 8015 | 1555014_x_at | 3.97E−04 | — |
| 8015 | 1569192_at | 1.20E−04 | — |
| 8015 | 242383_at | 1.00E−04 | — |
| 8016 | 1559780_at | 5.00E−04 | — |
| 8083 | 216152_at | 4.00E−04 | — |
| 8132 | 234135_x_at | 0.001735 | — |
| 8147 | 1565692_at | 2.87E−04 | — |
| 8147 | 1556442_x_at | 4.21E−04 | — |
| 8147 | 233622_x_at | 4.19E−05 | — |
| 8147 | 233270_x_at | 0.001256 | — |
| 8147 | 1566501_at | 4.00E−05 | — |
| 8156 | 233239_at | 4.45E−04 | — |
| 8156 | 217105_at | 4.96E−04 | — |
| 8156 | 217102_at | 4.96E−04 | — |
| 8156 | 1566146_x_at | 3.09E−04 | — |
| 8156 | 1566144_at | 3.09E−04 | — |
| 8165 | 1559497_at | 0.0025 | — |
| 8182 | 1570639_at | 5.00E−05 | — |
| 10151 | 1561154_at | 6.25E−04 | — |
| 10151 | 237124_at | 0.003333 | — |
| 8221 | 242995_at | 0.005 | — |
| 8236 | 239088_at | 0.003 | — |
| 8246 | 1562076_at | 3.88E−05 | — |
| 8247 | 1563279_at | 5.00E−04 | — |
| 8247 | 1570483_at | 0.002 | — |
| 8252 | 1565874_at | 0.004 | — |
| 8264 | 230118_at | 4.20E−04 | — |
| 8264 | 1563154_at | 7.80E−04 | — |
| 5618 | 242390_at | 1.00E−05 | — |
| 8270 | 1560868_s_at | 3.00E−06 | — |
| 8270 | 222247_at | 5.00E−04 | — |
| 8303 | 205183_at | 0.001696 | — |
| 8303 | 235399_at | 6.26E−04 | — |
| 8303 | 231937_at | 6.01E−04 | — |
| 8303 | 1565837_at | 4.06E−05 | — |
| 8303 | 1563345_at | 8.88E−04 | — |
| 8303 | 1562811_at | 3.00E−04 | — |
| 8303 | 1561156_at | 2.87E−04 | — |
| 8303 | 1557335_at | 3.69E−04 | — |
| 8303 | 242476_at | 7.00E−05 | — |
| 8303 | 206936_x_at | 1.69E−04 | — |
| 8306 | 243667_at | 0.001051 | — |
| 8311 | 1562905_at | 3.17E−04 | — |
| 8311 | 217653_x_at | 9.02E−05 | — |
| 8311 | 233776_at | 4.99E−04 | — |
| 8318 | 222337_at | 0.00145 | — |
| 8319 | 1561107_at | 0.001 | — |
| 8320 | 207915_at | 3.27E−04 | — |
| 8324 | 242842_at | 5.15E−04 | — |
| 8355 | 243485_at | 5.00E−04 | — |
| 8366 | 222373_at | 1.44E−04 | — |
| 8366 | 1557301_a_at | 1.02E−04 | — |
| 8366 | 1561432_at | 1.00E−05 | — |
| 8366 | 241913_at | 4.00E−05 | — |
| 8366 | 215824_at | 4.80E−04 | — |
| 8366 | 1561591_at | 4.50E−04 | — |
| 8376 | 1569537_at | 3.00E−06 | — |
| 8407 | 241468_at | 3.00E−06 | — |
| 8430 | 232797_at | 0.002 | — |
| 8442 | 236327_at | 0.002 | — |
| 8453 | 233824_at | 6.67E−04 | — |
| 8453 | 1566535_at | 1.00E−06 | — |
| 8453 | 243203_at | 1.00E−06 | — |
| 8453 | 233904_at | 1.00E−05 | — |
| 8462 | 1555392_at | 4.00E−05 | — |
| 8463 | 1569354_at | 5.00E−04 | — |
| 8490 | 237023_at | 3.00E−06 | — |
| 8514 | 233716_at | 4.47E−06 | — |
| 8568 | 242369_x_at | 4.00E−05 | — |
| 8614 | 243577_at | 0.002 | — |
| 8672 | 235872_at | 1.00E−07 | — |
| 8672 | 239995_at | 6.00E−04 | — |
| 8678 | 230461_s_at | 4.00E−10 | — |
| 8699 | 1565722_at | 4.10E−05 | — |
| 8755 | 1566688_at | 0.002 | — |
| 8763 | 237868_x_at | 0.001008 | — |
| 8763 | 1556683_x_at | 3.73E−04 | — |
| 8763 | 217580_x_at | 8.70E−04 | — |
| 8763 | 217579_x_at | 8.70E−04 | — |
| 8763 | 1569181_x_at | 5.00E−05 | — |
| 8763 | 1569180_at | 5.00E−05 | — |
| 8763 | 240205_x_at | 1.00E−04 | — |
| 8763 | 233867_at | 6.50E−05 | — |
| 8763 | 1561149_at | 6.00E−04 | — |
| 8766 | 215956_at | 7.23E−05 | — |
| 8766 | 1561741_at | 1.88E−04 | — |
| 8788 | 233115_at | 0.003 | — |
| 8791 | 217126_at | 7.00E−06 | — |
| 8791 | 1563076_x_at | 7.00E−06 | — |
| 8791 | 1563075_s_at | 7.00E−06 | — |
| 8791 | 1561713_at | 7.00E−06 | — |
| 8792 | 217706_at | 3.62E−04 | — |
| 8806 | 236923_x_at | 0.002 | — |
| 8819 | 1561642_at | 0.001 | — |
| 8819 | 232749_at | 2.95E−04 | — |
| 8823 | 216112_at | 1.00E−05 | — |
| 8829 | 1561417_x_at | 4.00E−06 | — |
| 8829 | 1561415_at | 4.00E−06 | — |
| 8839 | 233248_at | 2.00E−04 | — |
| 8846 | 208061_at | 0.003 | — |
| 8885 | 240978_at | 1.00E−05 | — |
| 8892 | 1562145_at | 6.68E−04 | — |
| 8900 | 238570_at | 0.002 | — |
| 67 | 1560540_x_at | 7.30E−04 | — |
| 67 | 1557712_x_at | 8.26E−04 | — |
| 67 | 1557710_at | 8.26E−04 | — |
| 67 | 242609_x_at | 0.001114 | — |
| 67 | 1566768_at | 5.89E−04 | — |
| 5935 | 1561914_at | 2.30E−04 | — |
| 8994 | 1561562_at | 0.001203 | — |
| 5937 | 243037_at | 7.60E−04 | — |
| 5937 | 234148_at | 5.08E−04 | — |
| 5937 | 1564148_at | 2.46E−04 | — |
| 5937 | 1562771_at | 8.39E−05 | — |
| 5937 | 1560230_at | 5.79E−04 | — |
| 5937 | 215406_at | 2.19E−04 | — |
| 5937 | 234643_x_at | 8.71E−04 | — |
| 5937 | 209446_s_at | 8.29E−04 | — |
| 5937 | 1564789_at | 1.38E−04 | — |
| 5937 | 1563303_at | 2.62E−04 | — |
| 5937 | 1560774_at | 0.001013 | — |
| 5937 | 1555617_x_at | 4.07E−05 | — |
| 5937 | 1555616_at | 4.07E−05 | — |
| 5937 | 215597_x_at | 2.78E−04 | — |
| 5937 | 229255_x_at | 2.20E−04 | — |
| 5937 | 234478_at | 4.79E−04 | — |
| 9005 | 234654_at | 1.00E−11 | — |
| 9010 | 239603_x_at | 1.00E−04 | — |
| 9051 | 1562925_at | 5.05E−04 | — |
| 9051 | 216691_at | 1.70E−04 | — |
| 9051 | 239366_at | 5.00E−04 | — |
| 9051 | 1559377_at | 3.05E−04 | — |
| 9101 | 1562586_at | 1.00E−07 | — |
| 897 | 1560349_at | 3.00E−06 | — |
| 915 | 242643_x_at | 0.001338 | — |
| 932 | 1564568_at | 3.61E−04 | — |
| 932 | 241265_x_at | 8.10E−05 | — |
| 959 | 215872_at | 5.50E−05 | — |
| 959 | 234535_at | 5.50E−05 | — |
| 959 | 1560673_at | 5.50E−05 | — |
| 959 | 232773_at | 2.70E−04 | — |
| 1034 | 207875_at | 0.00125 | — |
| 1040 | 242752_at | 7.00E−06 | — |
| 1043 | 207436_x_at | 1.09E−04 | — |
| 1043 | 1563934_at | 1.09E−04 | — |
| 1043 | 217531_at | 6.43E−04 | — |
| 1043 | 216642_at | 1.93E−04 | — |
| 1043 | 242813_at | 1.41E−04 | — |
| 1043 | 217288_at | 2.41E−04 | — |
| 1043 | 216769_x_at | 1.32E−04 | — |
| 1043 | 233218_at | 1.65E−04 | — |
| 1043 | 1560347_at | 2.66E−04 | — |
| 1043 | 1559210_at | 1.91E−04 | — |
| 1043 | 214989_x_at | 9.53E−04 | — |
| 1043 | 1566703_at | 7.69E−04 | — |
| 1043 | 1560721_at | 8.00E−04 | — |
| 1043 | 1557864_x_at | 2.05E−04 | — |
| 1043 | 1564773_x_at | 4.86E−04 | — |
| 1043 | 1557522_x_at | 6.59E−04 | — |
| 1043 | 1570331_at | 3.39E−05 | — |
| 1047 | 234501_x_at | 2.46E−05 | — |
| 1047 | 233390_at | 1.61E−04 | — |
| 1047 | 1559683_at | 1.87E−04 | — |
| 1047 | 217679_x_at | 6.92E−04 | — |
| 1047 | 1565740_at | 2.12E−04 | — |
| 1047 | 1560678_at | 3.35E−05 | — |
| 1047 | 1557468_at | 4.59E−04 | — |
| 1047 | 241268_x_at | 9.72E−04 | — |
| 1047 | 234156_at | 4.46E−04 | — |
| 1129 | 1569942_at | 6.13E−04 | — |
| 1129 | 239788_at | 4.74E−04 | — |
| 1129 | 1562541_at | 1.48E−04 | — |
| 1129 | 1559753_at | 1.48E−04 | — |
| 1129 | 231317_at | 2.60E−04 | — |
| 1129 | 243660_at | 4.00E−04 | — |
| 1138 | 217606_at | 5.00E−04 | — |
| 1177 | 241718_x_at | 5.10E−04 | — |
| 1201 | 1555373_at | 3.00E−05 | — |
| 1340 | 238743_at | 9.00E−04 | — |
| 1340 | 236628_at | 2.22E−04 | — |
| 1340 | 243442_x_at | 8.34E−04 | — |
| 1340 | 243866_x_at | 9.50E−04 | — |
| 1340 | 239195_at | 0.001325 | — |
| 1407 | 233038_at | 1.94E−04 | — |
| 1407 | 243207_at | 3.24E−04 | — |
| 1407 | 216745_x_at | 7.13E−04 | — |
| 1407 | 233832_at | 0.00202 | — |
| 1407 | 233817_at | 0.001629 | — |
| 1407 | 1563482_at | 0.001185 | — |
| 1407 | 238611_at | 2.34E−04 | — |
| 1407 | 232875_at | 1.61E−04 | — |
| 6009 | 1559523_at | 3.40E−04 | — |
| 6009 | 1559702_at | 0.0025 | — |
| 1428 | 1566518_at | 8.33E−05 | — |
| 1428 | 1561573_at | 2.00E−04 | — |
| 1428 | 1563297_s_at | 6.00E−05 | — |
| 1443 | 1564056_at | 6.00E−05 | — |
| 1444 | 1559452_a_at | 8.80E−04 | — |
| 1458 | 216562_at | 0.003 | — |
| 1471 | 234222_at | 1.44E−06 | — |
| 1515 | 210723_x_at | 1.97E−04 | — |
| 1516 | 1564294_at | 5.89E−04 | — |
| 1562 | 1568869_at | 0.001233 | — |
| 1562 | 221130_s_at | 0.001196 | — |
| 1562 | 217639_at | 0.00118 | — |
| 1562 | 217479_at | 4.94E−04 | — |
| 1562 | 215650_at | 8.03E−04 | — |
| 1562 | 211369_at | 0.001015 | — |
| 1562 | 221184_at | 0.001139 | — |
| 1562 | 215893_x_at | 7.72E−04 | — |
| 1562 | 232867_at | 5.30E−04 | — |
| 1562 | 232743_at | 0.001158 | — |
| 1562 | 1568754_at | 0.0015 | — |
| 1562 | 1565705_x_at | 6.10E−04 | — |
| 1562 | 1561463_at | 0.001002 | — |
| 1562 | 1557849_at | 8.84E−04 | — |
| 1562 | 1556815_at | 9.56E−04 | — |
| 6025 | 244843_x_at | 1.00E−06 | — |
| 6038 | 230642_at | 5.50E−05 | — |
| 9320 | 242936_at | 3.20E−04 | — |
| 9320 | 1565628_at | 0.001505 | — |
| 9330 | 216499_at | 2.01E−04 | — |
| 9338 | 1570205_at | 4.12E−04 | — |
| 9338 | 233721_x_at | 5.22E−05 | — |
| 9338 | 215846_at | 5.53E−05 | — |
| 9338 | 210679_x_at | 1.48E−04 | — |
| 9338 | 215742_at | 2.27E−04 | — |
| 9338 | 1564840_at | 3.95E−04 | — |
| 9338 | 1557644_at | 5.97E−05 | — |
| 9338 | 235123_at | 2.16E−04 | — |
| 9338 | 1570198_x_at | 1.35E−04 | — |
| 9338 | 1564336_at | 1.02E−04 | — |
| 9338 | 221176_x_at | 2.83E−04 | — |
| 9338 | 1562947_x_at | 1.21E−05 | — |
| 9338 | 1562946_at | 1.21E−05 | — |
| 9338 | 215609_at | 5.15E−04 | — |
| 9342 | 242924_at | 1.00E−06 | — |
| 9345 | 1564767_at | 4.31E−06 | — |
| 9345 | 1562036_at | 2.58E−04 | — |
| 9345 | 232995_at | 0.001864 | — |
| 9345 | 222306_at | 1.81E−04 | — |
| 9345 | 234046_at | 9.03E−05 | — |
| 9345 | 233677_at | 2.19E−05 | — |
| 9345 | 1561600_at | 2.48E−05 | — |
| 9345 | 1561181_at | 2.23E−05 | — |
| 9345 | 1557631_at | 1.28E−04 | — |
| 9345 | 242501_at | 4.12E−04 | — |
| 9345 | 216051_x_at | 2.71E−04 | — |
| 9345 | 244145_at | 1.08E−04 | — |
| 9352 | 239900_x_at | 2.29E−04 | — |
| 9352 | 235028_at | 1.38E−04 | — |
| 9352 | 232919_at | 9.26E−04 | — |
| 9352 | 233994_at | 2.04E−04 | — |
| 9352 | 242815_x_at | 1.01E−05 | — |
| 9352 | 234047_at | 3.65E−05 | — |
| 9352 | 1564753_at | 2.38E−04 | — |
| 9352 | 240529_at | 1.66E−04 | — |
| 9352 | 1566582_x_at | 0.00101 | — |
| 9354 | 215372_x_at | 5.50E−05 | — |
| 9358 | 231888_at | 4.00E−08 | — |
| 9365 | 216789_at | 3.63E−04 | — |
| 9365 | 233931_at | 3.81E−06 | — |
| 9365 | 239409_at | 4.50E−05 | — |
| 9365 | 234423_x_at | 0.001618 | — |
| 9365 | 1561555_at | 8.70E−04 | — |
| 9365 | 1563033_x_at | 0.00125 | — |
| 9365 | 1563032_at | 0.00125 | — |
| 9396 | 1560443_at | 6.00E−04 | — |
| 9397 | 237488_at | 3.16E−05 | — |
| 5636 | 1569779_at | 3.09E−05 | — |
| 1582 | 1569539_at | 9.00E−04 | — |
| 1599 | 240541_at | 2.70E−04 | — |
| 1604 | 242025_at | 0.00201 | — |
| 1604 | 1566106_at | 0.001595 | — |
| 6061 | 239497_at | 9.00E−04 | — |
| 1665 | 242564_at | 0.003 | — |
| 1717 | 236801_at | 2.50E−04 | — |
| 1732 | 241848_x_at | 2.47E−04 | — |
| 1732 | 216811_at | 9.40E−05 | — |
| 1732 | 233041_x_at | 1.87E−04 | — |
| 1732 | 1569341_at | 2.60E−04 | — |
| 1732 | 232347_x_at | 4.39E−04 | — |
| 1732 | 1556595_at | 3.50E−04 | — |
| 1732 | 232622_at | 4.13E−04 | — |
| 1732 | 1557897_at | 2.13E−04 | — |
| 1750 | 210717_at | 0.002 | — |
| 1752 | 1570065_at | 0.002023 | — |
| 1784 | 1568867_x_at | 0.001107 | — |
| 1784 | 1568866_at | 0.001107 | — |
| 1784 | 232496_at | 4.91E−04 | — |
| 1885 | 232273_at | 0.001072 | — |
| 1904 | 243561_at | 0.003 | — |
| 1920 | 230350_at | 6.00E−04 | — |
| 1921 | 242612_at | 1.50E−04 | — |
| 1921 | 220913_at | 0.005 | — |
| 1921 | 243078_at | 0.005 | — |
| 1921 | 236235_at | 0.005 | — |
| 1943 | 239752_at | 2.50E−05 | — |
| 1964 | 239418_x_at | 1.00E−06 | — |
| 1964 | 1556345_s_at | 1.00E−06 | — |
| 1964 | 234439_at | 3.13E−04 | — |
| 1964 | 237398_at | 7.00E−04 | — |
| 98 | 235681_at | 0.003 | — |
| 2045 | 217443_at | 1.19E−04 | — |
| 2045 | 1558742_at | 5.20E−06 | — |
| 2045 | 234245_at | 0.00102 | — |
| 2045 | 1561829_at | 2.36E−04 | — |
| 2045 | 1557996_at | 6.18E−06 | — |
| 2045 | 240535_at | 3.40E−05 | — |
| 2045 | 239969_at | 1.50E−04 | — |
| 2045 | 231489_x_at | 9.51E−04 | — |
| 2045 | 239649_at | 5.00E−04 | — |
| 2045 | 1566232_at | 4.10E−04 | — |
| 2045 | 1566209_at | 0.001115 | — |
| 2089 | 217616_at | 9.58E−05 | — |
| 2089 | 217423_at | 9.17E−06 | — |
| 2089 | 241615_x_at | 1.38E−04 | — |
| 2108 | 233425_at | 1.00E−05 | — |
| 2108 | 242724_x_at | 5.50E−05 | — |
| 2110 | 244047_at | 0.001858 | — |
| 2110 | 208144_s_at | 1.64E−04 | — |
| 115 | 1561036_at | 2.30E−05 | — |
| 115 | 1560332_at | 1.19E−04 | — |
| 115 | 244588_at | 3.41E−04 | — |
| 115 | 1561346_at | 2.44E−04 | — |
| 115 | 1555303_at | 1.26E−05 | — |
| 115 | 222961_at | 3.16E−05 | — |
| 115 | 1569912_at | 5.38E−04 | — |
| 115 | 1563472_at | 3.17E−04 | — |
| 2136 | 1569767_at | 0.00125 | — |
| 2136 | 242658_at | 5.38E−04 | — |
| 117 | 1566716_at | 5.05E−04 | — |
| 2168 | 1566580_at | 5.00E−05 | — |
| 2173 | 1566527_at | 4.50E−06 | — |
| 123 | 243568_at | 6.57E−04 | — |
| 123 | 1562454_at | 0.001118 | — |
| 123 | 241303_x_at | 7.85E−04 | — |
| 123 | 1553462_at | 3.47E−05 | — |
| 123 | 240839_at | 4.63E−05 | — |
| 123 | 215607_x_at | 0.001023 | — |
| 2219 | 1557375_at | 2.00E−04 | — |
| 125 | 232962_x_at | 9.86E−05 | — |
| 130 | 1559821_at | 1.00E−05 | — |
| 2270 | 1557099_at | 2.20E−04 | — |
| 2274 | 237549_at | 0.001005 | — |
| 133 | 1566540_at | 1.00E−05 | — |
| 2300 | 1569673_at | 5.00E−04 | — |
| 2300 | 231231_at | 0.002 | — |
| 2315 | 242978_x_at | 7.58E−05 | — |
| 2322 | 237650_at | 0.002 | — |
| 2339 | 241865_at | 9.00E−05 | — |
| 2339 | 217293_at | 0.004 | — |
| 2341 | 1562168_at | 8.00E−04 | — |
| 2497 | 1563030_at | 4.00E−06 | — |
| 2582 | 1563611_at | 7.00E−05 | — |
| 2584 | 233181_at | 1.73E−04 | — |
| 2584 | 235800_at | 0.002 | — |
| 174 | 237539_at | 2.70E−05 | — |
| 174 | 243642_x_at | 3.20E−04 | — |
| 174 | 1563036_at | 3.01E−04 | — |
| 2700 | 1556521_a_at | 1.00E−13 | — |
| 2768 | 233912_x_at | 3.00E−06 | — |
| 2768 | 221179_at | 3.00E−06 | — |
| 2768 | 239868_at | 0.002 | — |
| 2774 | 1560264_at | 7.33E−06 | — |
| 183 | 242646_at | 5.05E−07 | — |
| 2790 | 1561595_x_at | 0.001767 | — |
| 2812 | 243984_at | 6.00E−05 | — |
| 2814 | 242117_at | 0.003 | — |
| 2814 | 221120_at | 0.003 | — |
| 2821 | 1568887_at | 0.004 | — |
| 2824 | 210920_x_at | 1.51E−04 | — |
| 2824 | 243931_at | 8.68E−06 | — |
| 2824 | 220700_at | 1.45E−04 | — |
| 2824 | 217643_x_at | 1.68E−04 | — |
| 2824 | 234814_at | 1.58E−04 | — |
| 2824 | 232714_at | 7.31E−05 | — |
| 2824 | 242740_at | 1.29E−04 | — |
| 2824 | 242413_at | 1.25E−04 | — |
| 2824 | 239333_x_at | 1.42E−04 | — |
| 2824 | 217570_x_at | 7.66E−06 | — |
| 2824 | 238829_at | 1.02E−04 | — |
| 2824 | 238672_at | 1.93E−04 | — |
| 2824 | 215204_at | 4.91E−05 | — |
| 2824 | 236060_at | 4.36E−05 | — |
| 2824 | 234214_at | 2.58E−04 | — |
| 2824 | 234193_at | 2.96E−04 | — |
| 2824 | 233358_at | 2.68E−04 | — |
| 2824 | 233289_at | 1.34E−04 | — |
| 2824 | 233154_at | 1.10E−04 | — |
| 2824 | 233037_at | 9.90E−06 | — |
| 2824 | 232723_at | 9.42E−05 | — |
| 2824 | 232626_at | 6.07E−04 | — |
| 2824 | 208238_x_at | 6.58E−04 | — |
| 2824 | 207756_at | 9.32E−05 | — |
| 2824 | 207730_x_at | 2.28E−04 | — |
| 2824 | 207481_at | 6.18E−05 | — |
| 2824 | 1570361_a_at | 7.15E−05 | — |
| 2824 | 1570100_at | 1.15E−04 | — |
| 2824 | 1569952_x_at | 1.60E−04 | — |
| 2824 | 1569772_x_at | 1.91E−04 | — |
| 2824 | 1569712_at | 3.89E−06 | — |
| 2824 | 1569577_x_at | 1.91E−04 | — |
| 2824 | 1566243_at | 1.01E−04 | — |
| 2824 | 1564937_at | 5.17E−04 | — |
| 2824 | 1563461_at | 3.98E−05 | — |
| 2824 | 1562890_at | 1.64E−05 | — |
| 2824 | 1562265_at | 5.68E−06 | — |
| 2824 | 1560792_at | 7.17E−05 | — |
| 2824 | 1559795_at | 2.07E−04 | — |
| 2824 | 1559652_at | 1.22E−04 | — |
| 2824 | 1557486_at | 3.55E−04 | — |
| 2824 | 1552975_x_at | 3.93E−05 | — |
| 2824 | 1552974_at | 3.88E−05 | — |
| 2824 | 240706_at | 6.70E−05 | — |
| 2824 | 210598_at | 1.89E−04 | — |
| 2824 | 243391_x_at | 3.66E−05 | — |
| 2824 | 216123_x_at | 2.75E−04 | — |
| 2824 | 214902_x_at | 2.18E−04 | — |
| 2824 | 236153_at | 2.18E−04 | — |
| 2824 | 234656_x_at | 2.73E−04 | — |
| 2824 | 234060_at | 2.06E−05 | — |
| 2824 | 1570496_at | 0.001087 | — |
| 2824 | 1565809_x_at | 4.64E−04 | — |
| 2824 | 233004_x_at | 1.02E−04 | — |
| 2824 | 216189_at | 5.87E−05 | — |
| 2824 | 234494_x_at | 9.65E−04 | — |
| 2824 | 216771_at | 4.53E−04 | — |
| 2824 | 215494_at | 7.60E−04 | — |
| 2824 | 235105_at | 7.08E−04 | — |
| 2824 | 1562823_at | 1.65E−04 | — |
| 2824 | 1559663_at | 2.37E−04 | — |
| 2824 | 241159_x_at | 0.001643 | — |
| 2824 | 215587_x_at | 4.58E−04 | — |
| 2824 | 234788_x_at | 8.93E−04 | — |
| 2824 | 1566953_x_at | 1.43E−04 | — |
| 2824 | 1566952_at | 1.43E−04 | — |
| 2824 | 215831_at | 1.08E−04 | — |
| 2824 | 215439_x_at | 1.60E−04 | — |
| 2824 | 214808_at | 3.12E−04 | — |
| 2824 | 234517_at | 8.58E−05 | — |
| 2824 | 234346_x_at | 4.24E−04 | — |
| 2824 | 1570295_at | 1.70E−04 | — |
| 2824 | 222170_at | 3.95E−04 | — |
| 2824 | 1561604_at | 1.87E−04 | — |
| 187 | 232894_at | 4.00E−05 | — |
| 187 | 229932_at | 4.00E−05 | — |
| 2836 | 1562642_at | 0.001 | — |
| 194 | 233014_at | 1.00E−04 | — |
| 2936 | 235575_at | 1.00E−08 | — |
| 2955 | 232803_at | 0.002 | — |
| 3000 | 231055_at | 2.00E−05 | — |
| 3002 | 1561732_at | 3.60E−04 | — |
| 211 | 1566809_a_at | 3.50E−04 | — |
| 3087 | 1563597_at | 0.003 | — |
| 10183 | 240000_at | 0.00145 | — |
| 3146 | 240485_at | 0.004 | — |
| 3167 | 236975_at | 2.00E−04 | — |
| 5652 | 1556486_at | 4.00E−05 | — |
| 5652 | 233768_at | 1.30E−04 | — |
| 5652 | 242024_at | 2.00E−04 | — |
| 5652 | 216625_at | 7.00E−04 | — |
| 5652 | 1570138_at | 0.001061 | — |
| 5652 | 1564820_at | 0.001567 | — |
| 3197 | 234527_at | 1.27E−08 | — |
| 3204 | 1562755_at | 1.00E−04 | — |
| 3215 | 232344_at | 6.50E−06 | — |
| 3277 | 1557232_at | 4.85E−04 | — |
| 3285 | 1559467_at | 1.00E−04 | — |
| 3287 | 229011_at | 7.75E−06 | — |
| 3322 | 216414_at | 4.00E−06 | — |
| 3348 | 217577_at | 5.00E−04 | — |
| 3381 | 232402_at | 0.003 | — |
| 3395 | 242174_at | 0.001315 | — |
| 3395 | 236571_at | 0.001508 | — |
| 3402 | 216425_at | 0.0012 | — |
| 3402 | 234492_at | 4.85E−04 | — |
| 250 | 1569603_at | 4.00E−07 | — |
| 3465 | 243876_at | 2.82E−05 | — |
| 3465 | 242223_at | 1.09E−04 | — |
| 3465 | 235716_at | 7.78E−05 | — |
| 3465 | 1565877_at | 3.75E−05 | — |
| 3465 | 1565876_x_at | 3.75E−05 | — |
| 3465 | 1563519_at | 2.33E−05 | — |
| 3465 | 1560971_a_at | 2.18E−05 | — |
| 3465 | 1560798_at | 7.95E−05 | — |
| 3465 | 233315_at | 3.87E−04 | — |
| 3465 | 234283_at | 5.38E−05 | — |
| 3465 | 233128_at | 1.48E−04 | — |
| 3465 | 216110_x_at | 7.77E−04 | — |
| 3465 | 234574_at | 1.88E−04 | — |
| 3465 | 216729_at | 2.99E−04 | — |
| 3465 | 1557630_s_at | 5.00E−04 | — |
| 3465 | 230663_at | 0.001507 | — |
| 3465 | 220696_at | 8.40E−04 | — |
| 3465 | 234020_x_at | 1.06E−04 | — |
| 3465 | 1557555_at | 1.07E−04 | — |
| 3465 | 1562863_at | 2.78E−04 | — |
| 3465 | 234059_at | 6.83E−05 | — |
| 3509 | 232335_at | 4.00E−08 | — |
| 3509 | 216669_at | 2.31E−04 | — |
| 3572 | 244274_at | 0.003 | — |
| 3577 | 238495_at | 0.004 | — |
| 3588 | 234182_at | 2.88E−04 | — |
| 3588 | 1563704_at | 2.43E−05 | — |
| 3588 | 1569703_a_at | 1.53E−05 | — |
| 3588 | 215763_at | 3.16E−04 | — |
| 3588 | 1561612_at | 5.17E−04 | — |
| 3588 | 238248_at | 0.00102 | — |
| 264 | 1565856_at | 8.00E−05 | — |
| 264 | 233515_at | 2.00E−05 | — |
| 264 | 1569208_a_at | 2.00E−05 | — |
| 264 | 1570244_at | 4.26E−04 | — |
| 264 | 1562035_at | 3.00E−04 | — |
| 3592 | 242495_at | 1.04E−04 | — |
| 3592 | 243640_x_at | 1.12E−04 | — |
| 3592 | 243147_x_at | 7.11E−05 | — |
| 3592 | 242448_at | 7.33E−04 | — |
| 3592 | 1563114_at | 7.33E−04 | — |
| 3592 | 216101_at | 8.32E−04 | — |
| 3609 | 239735_at | 2.08E−04 | — |
| 3620 | 1563329_s_at | 2.45E−06 | — |
| 3620 | 1559372_at | 4.32E−04 | — |
| 3620 | 235407_at | 1.28E−04 | — |
| 3624 | 237410_x_at | 0.00125 | — |
| 3628 | 239408_at | 2.00E−05 | — |
| 3628 | 242588_at | 3.00E−04 | — |
| 3628 | 240271_at | 3.00E−04 | — |
| 3646 | 1559259_at | 2.78E−04 | — |
| 3660 | 1566880_at | 5.00E−04 | — |
| 3660 | 233094_at | 5.00E−04 | — |
| 3676 | 222356_at | 6.57E−05 | — |
| 3676 | 1559709_at | 3.66E−04 | — |
| 3697 | 237561_x_at | 0.001022 | — |
| 3842 | 242695_at | 0.002 | — |
| 3857 | 232997_at | 4.20E−04 | — |
| 293 | 237245_at | 6.16E−04 | — |
| 3906 | 1557409_at | 0.001694 | — |
| 3913 | 235594_at | 1.00E−06 | — |
| 3935 | 229206_at | 3.03E−04 | — |
| 3964 | 241135_at | 7.00E−05 | — |
| 147 | 237580_at | 2.90E−05 | — |
| 3991 | 1563750_at | 0.00125 | — |
| 4012 | 233420_at | 1.10E−05 | — |
| 4033 | 1561654_at | 4.00E−05 | — |
| 4064 | 240630_at | 0.002 | — |
| 4161 | 234087_at | 4.00E−04 | — |
| 4161 | 233373_at | 0.004 | — |
| 324 | 241835_at | 1.00E−04 | — |
| 4220 | 234590_x_at | 1.27E−04 | — |
| 4220 | 1566848_x_at | 6.92E−04 | — |
| 4247 | 241388_at | 0.002 | — |
| 4286 | 217533_x_at | 4.00E−08 | — |
| 4286 | 233505_at | 0.001 | — |
| 4292 | 238738_at | 3.38E−04 | — |
| 5663 | 215577_at | 2.00E−04 | — |
| 4255 | 1559449_a_at | 7.00E−05 | — |
| 4255 | 1556685_at | 0.002 | — |
| 4267 | 216067_at | 3.00E−06 | — |
| 4267 | 224056_at | 2.89E−04 | — |
| 4268 | 241207_at | 6.50E−06 | — |
| 4268 | 208185_x_at | 2.13E−06 | — |
| 4268 | 1569167_at | 1.43E−04 | — |
| 4268 | 1569916_at | 6.50E−06 | — |
| 4268 | 232906_at | 3.80E−04 | — |
| 4268 | 236215_at | 2.15E−05 | — |
| 4317 | 242878_at | 1.87E−04 | — |
| 4331 | 238913_at | 1.70E−05 | — |
| 4331 | 1569275_at | 6.29E−04 | — |
| 4331 | 233142_at | 6.75E−05 | — |
| 4331 | 214388_at | 3.67E−04 | — |
| 4331 | 233407_at | 1.07E−04 | — |
| 4331 | 1558474_at | 0.001077 | — |
| 4331 | 239311_at | 4.63E−04 | — |
| 4331 | 1560405_at | 9.50E−04 | — |
| 4331 | 232889_at | 0.001033 | — |
| 4331 | 228623_at | 0.001033 | — |
| 345 | 220719_at | 3.72E−04 | — |
| 345 | 234153_at | 2.01E−04 | — |
| 345 | 233779_x_at | 0.002833 | — |
| 345 | 242535_at | 1.05E−04 | — |
| 345 | 1565802_at | 3.91E−04 | — |
| 345 | 232742_at | 7.23E−04 | — |
| 345 | 236549_x_at | 0.002 | — |
| 4392 | 234572_at | 2.36E−04 | — |
| 4415 | 1559810_at | 3.00E−06 | — |
| 4482 | 233922_at | 0.00124 | — |
| 4579 | 224105_x_at | 2.70E−04 | — |
| 4579 | 233739_at | 3.17E−04 | — |
| 4579 | 234565_x_at | 1.23E−04 | — |
| 4579 | 1563241_at | 4.35E−04 | — |
| 4579 | 1558078_at | 1.58E−04 | — |
| 4579 | 234382_x_at | 5.35E−04 | — |
| 4579 | 232363_at | 3.41E−04 | — |
| 4579 | 1562661_at | 2.53E−04 | — |
| 4579 | 242859_at | 2.24E−04 | — |
| 4579 | 241867_at | 7.35E−05 | — |
| 4579 | 210230_at | 0.00176 | — |
| 4579 | 214807_at | 1.53E−04 | — |
| 4579 | 1569491_at | 2.11E−04 | — |
| 4579 | 231212_x_at | 3.78E−04 | — |
| 4583 | 208301_at | 3.16E−04 | — |
| 4599 | 238016_s_at | 3.00E−06 | — |
| 4621 | 216630_at | 9.78E−05 | — |
| 4677 | 234122_at | 5.00E−07 | — |
| 378 | 234224_at | 1.78E−04 | — |
| 378 | 1564905_at | 1.45E−04 | — |
| 378 | 217713_x_at | 3.16E−04 | — |
| 378 | 215083_at | 7.84E−04 | — |
| 378 | 229949_at | 6.00E−04 | — |
| 378 | 1566763_at | 6.00E−04 | — |
| 4737 | 1557584_at | 0.001525 | — |
| 4793 | 242886_at | 1.85E−05 | — |
| 4793 | 242256_x_at | 6.22E−05 | — |
| 4793 | 234789_at | 1.66E−04 | — |
| 4793 | 1569540_at | 1.89E−04 | — |
| 4793 | 222320_at | 1.42E−04 | — |
| 4793 | 1570409_x_at | 6.23E−04 | — |
| 4793 | 1570408_at | 2.83E−05 | — |
| 4793 | 1558385_at | 4.05E−05 | — |
| 4793 | 1554972_at | 1.40E−05 | — |
| 4793 | 232665_x_at | 1.02E−04 | — |
| 4793 | 1570196_at | 1.37E−05 | — |
| 4793 | 215373_x_at | 9.94E−05 | — |
| 4793 | 222207_x_at | 2.17E−04 | — |
| 4808 | 215837_x_at | 2.30E−04 | — |
| 4808 | 215197_at | 6.58E−05 | — |
| 4808 | 234578_at | 1.35E−04 | — |
| 4808 | 233900_at | 1.92E−04 | — |
| 4808 | 1562063_x_at | 2.07E−04 | — |
| 4808 | 1562062_at | 9.92E−05 | — |
| 4808 | 1558672_at | 1.58E−04 | — |
| 4808 | 1555718_x_at | 7.98E−05 | — |
| 4808 | 1555717_at | 7.98E−05 | — |
| 4808 | 217524_x_at | 1.84E−04 | — |
| 4808 | 232495_x_at | 2.26E−04 | — |
| 4814 | 207487_at | 8.40E−04 | — |
| 4859 | 230757_at | 7.00E−07 | — |
| 4906 | 216675_at | 4.00E−04 | — |
| 4906 | 244637_at | 2.70E−04 | — |
| 4906 | 235662_at | 0.002 | — |
| 4917 | 224546_at | 9.00E−04 | — |
| 4950 | 233765_at | 7.00E−18 | — |
| 4964 | 1560258_a_at | 0.002 | — |
| 4973 | 233272_at | 5.00E−04 | — |
| 4995 | 1555374_at | 0.002 | — |
| 5016 | 232544_at | 0.001685 | — |
| 5016 | 241097_at | 6.70E−04 | — |
| 5016 | 244482_at | 0.001 | — |
| 5016 | 217164_at | 0.001492 | — |
| 5016 | 1557151_at | 0.002015 | — |
| 5016 | 244869_at | 0.00121 | — |
| 5043 | 216496_s_at | 2.16E−04 | — |
| 5043 | 1561924_at | 4.60E−04 | — |
| 5043 | 1562793_at | 4.75E−04 | — |
| 5043 | 241094_at | 2.72E−04 | — |
| 5043 | 216144_at | 3.51E−04 | — |
| 5043 | 233448_s_at | 1.85E−04 | — |
| 5043 | 232778_at | 1.63E−04 | — |
| 5043 | 231992_x_at | 2.36E−04 | — |
| 5043 | 208162_s_at | 5.22E−04 | — |
| 5043 | 1570013_at | 3.68E−04 | — |
| 5043 | 1566725_at | 2.39E−04 | — |
| 5043 | 1566459_at | 2.89E−04 | — |
| 5043 | 1563561_at | 7.21E−04 | — |
| 5043 | 1557538_at | 1.96E−04 | — |
| 5053 | 1570177_at | 4.00E−10 | — |
| 5069 | 1561581_at | 7.00E−07 | — |
| 416 | 1557224_at | 1.34E−04 | — |
| 416 | 242645_at | 4.25E−04 | — |
| 416 | 1555664_at | 4.46E−04 | — |
| 416 | 242950_x_at | 3.77E−04 | — |
| 416 | 216645_at | 2.49E−04 | — |
| 416 | 234170_at | 1.77E−04 | — |
| 416 | 233081_at | 1.59E−04 | — |
| 416 | 1566887_x_at | 2.46E−04 | — |
| 416 | 1559509_at | 6.01E−05 | — |
| 416 | 1557135_at | 2.70E−04 | — |
| 5127 | 1566279_at | 2.00E−05 | — |
| 5127 | 1566278_at | 2.00E−05 | — |
| 5127 | 222358_x_at | 8.00E−04 | — |
| 5170 | 240971_x_at | 7.20E−04 | — |
| 5170 | 237451_x_at | 7.94E−04 | — |
| 5170 | 233616_at | 7.96E−04 | — |
| 5170 | 232511_at | 2.73E−04 | — |
| 5170 | AFFX- | 7.67E−05 | — |
| | hum_alu_at | | |
| 5170 | 1569770_x_at | 1.09E−04 | — |
| 5170 | 1569761_x_at | 1.60E−04 | — |
| 5170 | 1569760_at | 1.60E−04 | — |
| 5170 | 1569409_x_at | 5.52E−04 | — |
| 5170 | 1562358_at | 2.14E−04 | — |
| 5170 | 215604_x_at | 2.89E−04 | — |
| 5170 | 215137_at | 1.68E−04 | — |
| 5170 | 233478_at | 4.25E−04 | — |
| 5170 | 232840_at | 6.11E−05 | — |
| 5170 | 1557758_at | 5.87E−04 | — |
| 5170 | 215182_x_at | 2.97E−04 | — |
| 5170 | 241680_at | 6.47E−04 | — |
| 5170 | 1562579_at | 4.21E−04 | — |
| 5170 | 1569929_at | 0.001012 | — |
| 5170 | 215200_x_at | 3.75E−04 | — |
| 5170 | 234113_at | 4.46E−04 | — |
| 5170 | 1566870_at | 3.57E−04 | — |
| 5170 | 1557325_at | 3.74E−04 | — |
| 5170 | 1556931_at | 3.18E−04 | — |
| 5170 | 1555117_at | 4.99E−04 | — |
| 5170 | 220856_x_at | 2.61E−04 | — |
| 5170 | 217481_x_at | 4.75E−04 | — |
| 5170 | 217052_x_at | 3.31E−04 | — |
| 5170 | 234833_at | 5.55E−04 | — |
| 5170 | 234084_x_at | 3.04E−04 | — |
| 5170 | 233427_x_at | 2.84E−04 | — |
| 5170 | 233027_at | 4.31E−04 | — |
| 5170 | 1563052_at | 3.25E−04 | — |
| 5170 | 1560638_a_at | 5.46E−04 | — |
| 5170 | 1560026_at | 4.42E−04 | — |
| 5170 | 210698_at | 4.31E−04 | — |
| 5170 | 1558449_at | 3.02E−04 | — |
| 5170 | 1554723_x_at | 8.60E−05 | — |
| 5170 | 234765_at | 5.91E−04 | — |
| 5170 | 1557908_at | 8.45E−04 | — |
| 5170 | 216858_x_at | 5.08E−04 | — |
| 5170 | 1570645_at | 4.08E−04 | — |
| 5170 | 1564794_at | 3.68E−04 | — |
| 5170 | 1562553_at | 0.002375 | — |
| 5216 | 1565576_at | 6.62E−04 | — |
| 5216 | 1557842_at | 4.35E−04 | — |
| 5328 | 238301_at | 4.17E−05 | — |
| 5328 | 239410_at | 3.73E−04 | — |
| 5328 | 216463_at | 2.11E−05 | — |
| 5328 | 234805_at | 1.44E−04 | — |
| 5328 | 224086_at | 6.20E−05 | — |
| 5328 | 222370_x_at | 9.19E−05 | — |
| 5328 | 222319_at | 2.67E−04 | — |
| 5328 | 220814_at | 1.42E−04 | — |
| 5328 | 242113_at | 7.25E−05 | — |
| 5328 | 216692_at | 2.00E−05 | — |
| 5328 | 216621_at | 5.46E−05 | — |
| 5328 | 216612_x_at | 1.64E−04 | — |
| 5328 | 215958_at | 5.29E−05 | — |
| 5328 | 215928_at | 4.00E−05 | — |
| 5328 | 215370_at | 1.84E−04 | — |
| 5328 | 215314_at | 2.30E−05 | — |
| 5328 | 215163_at | 7.09E−05 | — |
| 5328 | 215128_at | 1.61E−04 | — |
| 5328 | 234247_at | 6.65E−05 | — |
| 5328 | 233702_x_at | 1.41E−04 | — |
| 5328 | 233258_at | 5.43E−05 | — |
| 5328 | 233235_x_at | 5.81E−05 | — |
| 5328 | 233017_x_at | 3.04E−04 | — |
| 5328 | 230580_at | 1.48E−04 | — |
| 5328 | 1570491_at | 3.25E−04 | — |
| 5328 | 1570249_x_at | 2.45E−04 | — |
| 5328 | 1569551_at | 1.68E−05 | — |
| 5328 | 1569416_at | 6.00E−05 | — |
| 5328 | 1566098_at | 3.28E−04 | — |
| 5328 | 1565889_at | 2.57E−05 | — |
| 5328 | 1565888_at | 2.57E−05 | — |
| 5328 | 1564130_x_at | 3.00E−05 | — |
| 5328 | 1563012_x_at | 5.69E−05 | — |
| 5328 | 1562416_at | 2.84E−04 | — |
| 5328 | 1561879_at | 1.01E−04 | — |
| 5328 | 1561737_at | 9.22E−05 | — |
| 5328 | 1560560_at | 2.93E−04 | — |
| 5328 | 1560451_at | 1.65E−04 | — |
| 5328 | 1559037_a_at | 5.55E−05 | — |
| 5328 | 1558889_at | 5.15E−05 | — |
| 5328 | 243103_at | 4.54E−04 | — |
| 5328 | 239251_at | 4.10E−05 | — |
| 5328 | 240813_at | 1.26E−04 | — |
| 5328 | 238382_x_at | 5.28E−05 | — |
| 5328 | 236438_at | 2.08E−05 | — |
| 5328 | 232957_x_at | 1.68E−04 | — |
| 5328 | 232775_at | 3.29E−04 | — |
| 5328 | 216668_at | 3.97E−05 | — |
| 5328 | 207953_at | 1.28E−04 | — |
| 5328 | 222299_x_at | 5.28E−05 | — |
| 5328 | 216524_x_at | 1.40E−04 | — |
| 5328 | 216043_x_at | 1.15E−04 | — |
| 5328 | 216628_at | 2.23E−05 | — |
| 5328 | 244677_at | 4.28E−05 | — |
| 5328 | 244172_at | 6.73E−04 | — |
| 5328 | 222295_x_at | 1.40E−04 | — |
| 5328 | 217490_at | 6.38E−05 | — |
| 5328 | 215615_x_at | 2.29E−05 | — |
| 5328 | 215507_x_at | 7.58E−05 | — |
| 5328 | 215385_at | 5.96E−05 | — |
| 5328 | 233757_x_at | 3.10E−04 | — |
| 5328 | 233608_at | 7.64E−05 | — |
| 5328 | 233296_x_at | 1.45E−05 | — |
| 5328 | 233055_at | 4.49E−05 | — |
| 5328 | 208082_x_at | 1.23E−04 | — |
| 5328 | 244891_x_at | 4.24E−05 | — |
| 5328 | 244258_at | 3.38E−05 | — |
| 5328 | 222328_x_at | 5.08E−05 | — |
| 5328 | 222213_x_at | 1.48E−04 | — |
| 5328 | 242182_x_at | 2.65E−05 | — |
| 5328 | 242101_at | 3.95E−05 | — |
| 5328 | 241816_at | 2.79E−05 | — |
| 5328 | 241722_x_at | 1.58E−05 | — |
| 5328 | 240892_at | 5.07E−05 | — |
| 5328 | 239780_at | 5.26E−05 | — |
| 5328 | 217549_at | 3.31E−05 | — |
| 5328 | 216792_at | 5.55E−05 | — |
| 5328 | 1563080_at | 1.28E−04 | — |
| 5328 | 1560917_at | 3.38E−05 | — |
| 5328 | 221155_x_at | 6.31E−04 | — |
| 5328 | 215553_x_at | 1.80E−04 | — |
| 5328 | 211444_at | 2.23E−04 | — |
| 5328 | 208014_x_at | 4.06E−04 | — |
| 5328 | 1569597_at | 9.43E−05 | — |
| 5328 | 1566777_at | 8.66E−05 | — |
| 5328 | 1562071_at | 4.19E−05 | — |
| 5328 | 233120_at | 6.19E−05 | — |
| 5328 | 243658_at | 1.37E−04 | — |
| 5328 | 215595_x_at | 4.85E−04 | — |
| 5328 | 216472_at | 2.66E−05 | — |
| 5328 | 234773_x_at | 3.02E−04 | — |
| 5407 | 1564665_at | 6.00E−07 | — |
| 5409 | 241806_at | 1.57E−04 | — |
| 5416 | 214912_at | 2.40E−04 | — |
| 5461 | 217682_at | 1.00E−04 | — |
| 5496 | 234223_at | 0.002333 | — |
| 5496 | 1558237_x_at | 0.00228 | — |
| 5519 | 242630_at | 0.002 | — |
| 5522 | 244197_x_at | 1.67E−05 | — |
| 5526 | 242797_x_at | 0.002 | — |
| 5526 | 238967_at | 0.002 | — |
| 5526 | 1561896_at | 6.77E−04 | — |
| 468 | 240791_at | 0.001625 | — |
| 9451 | 233805_at | 9.16E−04 | — |
| 9462 | 1564338_at | 4.00E−06 | — |
| 9475 | 1570093_at | 4.06E−04 | — |
| 9475 | 241443_at | 6.25E−04 | — |
| 9475 | 216132_at | 0.001006 | — |
| 9475 | 243768_at | 6.73E−04 | — |
| 9477 | 220296_at | 9.16E−04 | — |
| 9508 | 217290_at | 0.003 | — |
| 9573 | 217695_x_at | 2.61E−04 | — |
| 9573 | 233553_at | 1.30E−04 | — |
| 9573 | 1569527_at | 2.66E−04 | — |
| 9573 | 1566266_at | 1.26E−04 | — |
| 9573 | 1566265_at | 1.26E−04 | — |
| 9573 | 220809_at | 1.19E−04 | — |
| 9573 | 217586_x_at | 4.94E−04 | — |
| 483 | 1561733_at | 3.25E−05 | — |
| 483 | 216613_at | 4.77E−05 | — |
| 483 | 239857_at | 1.22E−04 | — |
| 483 | 234786_at | 2.03E−04 | — |
| 483 | 240988_x_at | 3.81E−04 | — |
| 483 | 242266_x_at | 5.43E−04 | — |
| 483 | 1561918_at | 2.89E−04 | — |
| 483 | 1557478_at | 7.25E−06 | — |
| 483 | 1568755_a_at | 3.13E−05 | — |
| 483 | 1564472_at | 1.47E−04 | — |
| 9610 | 1561529_at | 1.00E−06 | — |
| 10123 | 234571_at | 3.00E−06 | — |
| 9620 | 243895_x_at | 8.67E−04 | — |
| 9628 | 236765_at | 3.36E−04 | — |
| 9628 | 233790_at | 3.89E−04 | — |
| 9628 | 233376_at | 7.04E−05 | — |
| 9628 | 1569478_s_at | 7.65E−05 | — |
| 9628 | 215196_at | 4.52E−05 | — |
| 9628 | 233627_at | 7.52E−04 | — |
| 9628 | 1554964_x_at | 4.34E−05 | — |
| 9628 | 1554963_at | 4.34E−05 | — |
| 9628 | 1570127_at | 2.19E−04 | — |
| 489 | 1564424_at | 3.20E−04 | — |
| 492 | 216166_at | 4.83E−04 | — |
| 492 | 1562898_at | 2.94E−04 | — |
| 492 | 233526_at | 4.85E−04 | — |
| 492 | 1561966_at | 1.01E−04 | — |
| 492 | 1561663_at | 0.001347 | — |
| 492 | 244711_at | 0.001155 | — |
| 492 | 215387_x_at | 5.47E−04 | — |
| 9731 | 244788_at | 7.00E−04 | — |
| 9731 | 241798_at | 0.002 | — |
| 9742 | 216448_at | 2.00E−06 | — |
| 9750 | 233412_x_at | 4.25E−04 | — |
| 9750 | 234274_at | 0.001599 | — |
| 9812 | 1561272_at | 2.15E−05 | — |
| 9831 | 234753_x_at | 1.50E−06 | — |
| 9859 | 1566715_at | 2.15E−05 | — |
| 524 | 222964_at | 4.23E−04 | — |
| 536 | 220874_at | 3.00E−06 | — |
| 483 | 1562338_at | 3.75E−04 | — |
| 9352 | 1557704_a_at | 7.00E−05 | — |
| 5731 | 232637_at | 4.00E−05 | — |
| 1043 | 215123_at | 0.001711 | 61E3.4 /// LOC100132247 /// LOC100652992 /// LOC613037 /// |
| | | | LOC728888 /// NPIPL3 |
| 6251 | 1564307_a_at | 4.36E−04 | A2ML1 |
| 5222 | 236394_at | 0.002 | A2MP1 |
| 2497 | 223593_at | 2.80E−04 | AADAT |
| 5328 | 243167_at | 8.15E−05 | ABCB5 |
| 796 | 1555323_at | 7.18E−05 | ABCB9 |
| 5652 | 242553_at | 6.00E−05 | ABCC3 |
| 5681 | 201872_s_at | 2.00E−04 | ABCE1 |
| 8303 | 209246_at | 4.82E−04 | ABCF2 |
| 5736 | 221927_s_at | 2.63E−04 | ABHD11 |
| 524 | 221815_at | 5.29E−05 | ABHD2 |
| 483 | 232862_at | 2.50E−05 | ABHD5 |
| 10151 | 216113_at | 0.001 | ABI2 |
| 8015 | 216103_at | 0.001167 | ACOT11 |
| 3465 | 1555321_at | 9.88E−05 | ACOT11 |
| 9578 | 220845_at | 1.00E−05 | ACOXL |
| 5117 | 234210_x_at | 3.00E−06 | ACTR2 |
| 4793 | 215590_x_at | 1.01E−04 | ACVR2B-AS1 |
| 2824 | 205745_x_at | 1.30E−04 | ADAM17 |
| 9573 | 1561451_a_at | 2.11E−04 | ADAM29 |
| 4793 | 233868_x_at | 0.001252 | ADAM33 |
| 4793 | 232570_s_at | 0.001302 | ADAM33 |
| 5736 | 1553234_at | 2.12E−04 | ADAMTS18 |
| 9352 | 220578_at | 1.35E−04 | ADAMTSL4 |
| 4116 | 219358_s_at | 7.88E−04 | ADAP2 |
| 5328 | 219384_s_at | 9.82E−05 | ADAT1 |
| 5736 | 235049_at | 2.54E−04 | ADCY1 |
| 559 | 213219_at | 0.001329 | ADCY2 |
| 6599 | 223781_x_at | 6.40E−04 | ADH4 |
| 2824 | 207175_at | 5.06E−05 | ADIPOQ |
| 639 | 205481_at | 1.00E−05 | ADORA1 |
| 1407 | 1555435_at | 1.57E−04 | AFF4 |
| 3588 | 1554618_at | 2.30E−05 | AGFG2 |
| 4593 | 1555610_at | 1.00E−06 | AGK |
| 559 | 223327_x_at | 2.32E−04 | AGSK1 /// LOC727751 /// LOC727849 |
| 3490 | 1563560_at | 0.002 | AHNAK |
| 1562 | 202820_at | 0.001783 | AHR |
| 796 | 212172_at | 1.91E−04 | AK2 |
| 7577 | 230630_at | 1.00E−04 | AK4 /// LOC100507855 |
| 4268 | 242515_x_at | 3.83E−04 | AKIP1 |
| 9345 | 1559982_s_at | 7.66E−05 | AKR1E2 |
| 5328 | 209646_x_at | 2.18E−04 | ALDH1B1 |
| 5328 | 211004_s_at | 7.22E−05 | ALDH3B1 |
| 796 | 243901_at | 1.27E−04 | ALDOB |
| 5731 | 1560733_at | 0.0025 | ALG5 |
| 2824 | 214707_x_at | 3.43E−04 | ALMS1 |
| 6249 | 207328_at | 0.001296 | ALOX15 |
| 4579 | 207133_x_at | 5.44E−04 | ALPK1 |
| 559 | 209425_at | 5.70E−05 | AMACR /// C1QTNF3 /// C1QTNF3-AMACR |
| 5170 | 222710_at | 1.88E−04 | AMIGO3 /// GMPPB |
| 10019 | 224667_x_at | 2.27E−04 | ANAPC16 |
| 7964 | 240363_at | 0.002 | ANK1 |
| 2110 | 1560370_x_at | 5.93E−04 | ANKH |
| 2110 | 1560369_at | 5.93E−04 | ANKH |
| 123 | 220076_at | 7.14E−05 | ANKH |
| 6375 | 210486_at | 4.67E−04 | ANKMY1 |
| 9768 | 1566147_a_at | 0.001 | ANKRD20A12P |
| 492 | 239196_at | 0.001012 | ANKRD22 |
| 5328 | 211717_at | 3.91E−05 | ANKRD40 |
| 1891 | 220144_s_at | 3.20E−06 | ANKRD5 |
| 8034 | 216601_at | 5.89E−04 | AOC4 |
| 9475 | 1555421_at | 5.42E−04 | AP1S3 |
| 1732 | 1558792_x_at | 1.79E−04 | AP2A1 |
| 3857 | 1570032_at | 2.34E−04 | AP3B2 |
| 3592 | 222517_at | 6.69E−05 | AP3M1 |
| 1562 | 235647_at | 6.66E−04 | AP4S1 |
| 2824 | 231858_x_at | 1.56E−04 | AP5B1 |
| 5491 | 1555448_at | 0.002 | AP5M1 |
| 5170 | 219706_at | 6.39E−04 | AP5S1 |
| 200 | 209913_x_at | 7.74E−05 | AP5Z1 |
| 2300 | 228101_at | 0.002 | APBA1 |
| 6249 | 212972_x_at | 0.001139 | APBB2 |
| 5328 | 210873_x_at | 4.09E−04 | APOBEC3A |
| 6249 | 206738_at | 8.89E−04 | APOC2 /// APOC4 /// APOC4-APOC2 |
| 6251 | 207262_at | 1.33E−04 | APOF |
| 5328 | 221653_x_at | 7.96E−05 | APOL2 |
| 2824 | 232814_x_at | 3.80E−05 | APOPT1 |
| 1732 | 211277_x_at | 1.70E−04 | APP |
| 416 | 215402_at | 1.14E−04 | APPBP2 |
| 6249 | 1561407_at | 0.001078 | ARAP2 |
| 379 | 225166_at | 2.00E−06 | ARHGAP18 |
| 4268 | 226056_at | 1.03E−04 | ARHGAP31 |
| 5170 | 233872_x_at | 7.66E−05 | ARHGAP5 |
| 796 | 232567_at | 4.54E−06 | ARHGAP8 /// PRR5-ARHGAP8 |
| 5170 | 1560445_x_at | 6.03E−04 | ARHGEF1 |
| 7645 | 1554703_at | 1.84E−04 | ARHGEF10 |
| 559 | 234129_at | 1.15E−04 | ARHGEF12 |
| 1043 | 217348_x_at | 1.53E−04 | ARHGEF15 |
| 5736 | 222121_at | 3.28E−04 | ARHGEF26 |
| 5736 | 233903_s_at | 4.85E−04 | ARHGEF26 |
| 4793 | 1553349_at | 1.58E−05 | ARID2 |
| 932 | 224322_at | 0.001944 | ARID4B |
| 7867 | 1555964_at | 2.55E−04 | ARL17A /// ARL17B /// LOC100294341 |
| 5889 | 213759_at | 0.0011 | ARL4C |
| 492 | 1554480_a_at | 2.67E−04 | ARMC10 |
| 345 | 234607_at | 1.00E−04 | ARRDC1 |
| 1949 | 218862_at | 2.00E−10 | ASB13 |
| 2110 | 1553253_at | 6.03E−04 | ASB16 |
| 5328 | 1560219_at | 4.29E−04 | ASB3 |
| 8965 | 219336_s_at | 0.001 | ASCC1 |
| 4253 | 221226_s_at | 0.002 | ASIC4 |
| 416 | 207288_at | 2.57E−04 | ASMTL-AS1 |
| 5170 | 241354_at | 2.24E−04 | ASMTL-AS1 |
| 5736 | 216197_at | 1.38E−04 | ATF7IP |
| 8246 | 216198_at | 3.81E−05 | ATF7IP |
| 2524 | 207728_at | 1.75E−06 | ATF7IP |
| 4906 | 231825_x_at | 6.36E−04 | ATF7IP |
| 2524 | 223677_at | 5.10E−04 | ATG10 |
| 5328 | 213026_at | 4.55E−04 | ATG12 |
| 6415 | 1553387_at | 3.00E−07 | ATM |
| 9338 | 210858_x_at | 1.28E−04 | ATM |
| 2824 | 1554631_at | 3.27E−04 | ATM |
| 6922 | 220920_at | 2.60E−05 | ATP10B |
| 6675 | 212362_at | 0.002 | ATP2A2 |
| 4579 | 222796_at | 6.44E−04 | ATP5J2-PTCD1 /// PTCD1 |
| 1340 | 1555114_at | 4.25E−04 | ATP6V0A2 |
| 663 | 212384_at | 2.55E−04 | ATP6V1G2-DDX39B /// DDX39B /// SNORD84 |
| 4808 | 214594_x_at | 4.02E−04 | ATP8B1 |
| 8303 | 1554704_at | 3.51E−04 | ATP8B3 |
| 6251 | 238723_at | 5.54E−04 | ATXN3 |
| 5328 | 233182_x_at | 1.55E−04 | ATXN3 |
| 9338 | 233333_x_at | 1.18E−04 | AVIL |
| 5736 | 1562391_at | 1.85E−04 | B3GALNT2 |
| 9354 | 1552834_at | 2.98E−06 | B3GNT6 |
| 4579 | 234938_at | 2.51E−04 | BACH1 |
| 5043 | 1555369_at | 3.05E−04 | BAGE |
| 5736 | 232885_at | 1.11E−04 | BBIP1 |
| 3620 | 232442_at | 9.24E−06 | BCAR1 |
| 4579 | 220588_at | 2.37E−04 | BCAS4 |
| 7472 | 1562313_at | 0.0011 | BCORP1 |
| 3145 | 224227_s_at | 3.00E−06 | BDP1 |
| 6064 | 224520_s_at | 3.00E−06 | BEST3 |
| 6306 | 232368_at | 8.00E−05 | BET3L |
| 5736 | 218056_at | 5.24E−04 | BFAR |
| 4793 | 230309_at | 4.25E−04 | BHMT2 |
| 483 | 210334_x_at | 2.25E−05 | BIRC5 |
| 5328 | 1569002_x_at | 1.19E−04 | BMP1 |
| 5328 | 1569001_at | 1.19E−04 | BMP1 |
| 5328 | 231873_at | 2.20E−05 | BMPR2 |
| 9041 | 1553072_at | 5.00E−09 | BNIPL |
| 1463 | 228430_at | 6.00E−04 | BOLA3-AS1 |
| 483 | 205750_at | 7.00E−05 | BPHL |
| 2824 | 208368_s_at | 1.90E−04 | BRCA2 |
| 8312 | 204520_x_at | 0.002 | BRD1 |
| 8312 | 215460_x_at | 0.002 | BRD1 |
| 345 | 242265_at | 9.72E−04 | BRD8 |
| 4104 | 225716_at | 0.003 | BRI3BP |
| 5736 | 221703_at | 3.61E−04 | BRIP1 |
| 1043 | 215349_at | 4.86E−04 | BTBD18 |
| 1043 | 217141_at | 1.10E−04 | BTBD7 |
| 3465 | 235819_at | 1.92E−04 | BTF3L4 |
| 5328 | 209770_at | 5.54E−05 | BTN3A1 |
| 8763 | 228434_at | 2.00E−04 | BTNL9 |
| 3465 | 215072_x_at | 1.77E−04 | C10orf137 |
| 3592 | 208115_x_at | 1.73E−04 | C10orf137 |
| 6674 | 1553844_a_at | 8.24E−05 | C10orf67 |
| 2244 | 1568978_s_at | 0.0022 | C11orf21 |
| 6811 | 1557801_x_at | 3.34E−04 | C11orf31 |
| 6811 | 1557799_at | 3.34E−04 | C11orf31 |
| 2541 | 1555365_x_at | 3.00E−06 | C11orf58 |
| 2541 | 1555364_at | 3.00E−06 | C11orf58 |
| 6358 | 238701_x_at | 1.34E−04 | C11orf92 |
| 5170 | 225836_s_at | 4.01E−04 | C12orf32 |
| 5328 | 1553821_at | 2.22E−04 | C12orf51 |
| 5736 | 223477_s_at | 1.48E−04 | C12orf65 |
| 2824 | 232650_at | 9.79E−05 | C12orf65 |
| 2045 | 1565765_x_at | 1.17E−04 | C13orf33 |
| 4405 | 202563_at | 2.00E−04 | C14orf1 |
| 5170 | 220084_at | 4.16E−04 | C14orf105 |
| 559 | 1553801_a_at | 3.28E−05 | C14orf126 |
| 6811 | 231859_at | 1.64E−04 | C14orf132 |
| 5328 | 1564816_at | 3.96E−05 | C14orf178 |
| 7984 | 1559097_at | 7.89E−04 | C14orf64 |
| 4808 | 1560499_at | 1.08E−04 | C14orf64 |
| 3592 | 1556588_at | 1.37E−04 | C15orf37 |
| 2108 | 232507_at | 2.70E−04 | C15orf41 |
| 8034 | 1560814_a_at | 0.00125 | C15orf57 |
| 3548 | 1558648_at | 1.00E−04 | C15orf57 |
| 3402 | 1565641_at | 0.001729 | C16orf45 |
| 483 | 1558557_at | 4.13E−05 | C16orf62 |
| 2824 | 220601_at | 2.36E−04 | C16orf70 |
| 378 | 1557206_at | 2.00E−04 | C17orf104 |
| 5328 | 1566480_x_at | 1.18E−04 | C17orf104 |
| 8453 | 1560703_at | 5.00E−05 | C17orf108 |
| 5328 | 1555981_at | 5.41E−05 | C17orf65 |
| 5569 | 221800_s_at | 0.003 | C17orf70 |
| 5569 | 228136_s_at | 0.003 | C17orf70 |
| 8453 | 225065_x_at | 6.67E−04 | C17orf76-AS1 /// SNORD49A /// SNORD49B /// SNORD65 |
| 1697 | 1553298_at | 1.00E−04 | C17orf77 |
| 7567 | 214816_x_at | 4.54E−04 | C19orf40 |
| 4579 | 224712_x_at | 4.17E−04 | C19orf42 |
| 9628 | 1553162_x_at | 5.93E−05 | C19orf55 |
| 67 | 232469_x_at | 9.47E−04 | C1orf191 |
| 5736 | 233693_at | 5.23E−05 | C1orf201 |
| 5170 | 1554246_at | 1.57E−04 | C1orf210 |
| 8936 | 1562909_at | 2.00E−04 | C1orf98 |
| 7567 | 242444_at | 2.12E−04 | C1QTNF6 |
| 9051 | 218983_at | 4.00E−05 | C1RL |
| 2089 | 233645_s_at | 2.04E−04 | C1RL |
| 4793 | 236618_at | 1.46E−04 | C20orf132 |
| 3330 | 1556721_at | 1.00E−05 | C20orf203 |
| 2300 | 231905_at | 1.00E−04 | C20orf96 |
| 4331 | 240801_at | 0.002 | C21orf37 |
| 2824 | 1557782_s_at | 3.25E−05 | C21orf49 |
| 3002 | 1553850_at | 0.001 | C21orf88 |
| 117 | 1555990_at | 5.00E−04 | C22orf42 |
| 125 | 1558756_at | 1.74E−04 | C2CD3 |
| 3330 | 220149_at | 0.00102 | C2orf54 |
| 492 | 230379_x_at | 4.35E−04 | C2orf56 |
| 492 | 1554176_a_at | 3.34E−04 | C3orf33 |
| 5328 | 220600_at | 1.45E−04 | C3orf75 |
| 5170 | 236240_at | 2.17E−04 | C4orf29 |
| 4793 | 224605_at | 1.06E−04 | C4orf3 |
| 7300 | 1565939_at | 5.50E−05 | C5orf22 |
| 560 | 1557828_a_at | 1.00E−04 | C5orf28 |
| 7984 | 1560128_x_at | 5.51E−04 | C5orf56 |
| 5638 | 1564276_at | 1.00E−05 | C5orf56 |
| 5736 | 1555095_at | 1.17E−04 | C6orf123 |
| 5328 | 238028_at | 1.89E−04 | C6orf132 |
| 5873 | 1554314_at | 4.73E−05 | C6orf141 |
| 3465 | 1553446_at | 0.001 | C6orf183 |
| 2274 | 236504_x_at | 5.00E−05 | C6orf52 |
| 9338 | 238504_at | 3.33E−04 | C6orf57 |
| 4083 | 224478_s_at | 4.00E−07 | C7orf50 |
| 3465 | 224758_at | 2.00E−04 | C7orf73 |
| 2650 | 229430_at | 1.00E−06 | C8orf46 |
| 5170 | 1564315_at | 1.29E−04 | C8orf49 |
| 6071 | 223697_x_at | 1.00E−05 | C9orf64 |
| 8270 | 238912_x_at | 5.05E−04 | C9orf85 |
| 5328 | 239222_at | 1.95E−04 | C9orf9 |
| 8015 | 221865_at | 9.67E−04 | C9orf91 |
| 5043 | 1555572_at | 2.35E−04 | CA6 |
| 8303 | 1558621_at | 1.61E−04 | CABLES1 |
| 123 | 1559420_x_at | 5.16E−04 | CACNB2 |
| 123 | 1559419_at | 5.16E−04 | CACNB2 |
| 416 | 215365_at | 3.60E−04 | CACNB2 |
| 1444 | 206384_at | 0.003 | CACNG3 |
| 9365 | 235834_at | 2.67E−04 | CALD1 |
| 2824 | 1554367_at | 1.63E−05 | CALHM1 |
| 3592 | 1566150_at | 3.97E−05 | CALML4 |
| 6456 | 229549_at | 0.001175 | CALU |
| 5328 | 243910_x_at | 2.12E−05 | CAND1 |
| 4268 | 226036_x_at | 2.06E−04 | CASP2 |
| 5170 | 211464_x_at | 7.32E−04 | CASP6 |
| 5328 | 209790_s_at | 2.43E−04 | CASP6 |
| 8147 | 213373_s_at | 2.41E−04 | CASP8 |
| 2045 | 1554786_at | 0.00152 | CASS4 |
| 5328 | 217588_at | 2.80E−05 | CATSPER2 /// CATSPER2P1 |
| 5731 | 244052_at | 0.001002 | CBR4 |
| 2045 | 1557488_at | 0.0015 | CBX3P2 |
| 5736 | 242324_x_at | 1.93E−04 | CCBE1 |
| 10019 | 1568850_at | 5.35E−06 | CCBP2 |
| 4482 | 239403_at | 0.001152 | CCDC120 |
| 8303 | 229082_at | 4.00E−06 | CCDC125 |
| 7605 | 1561477_at | 1.00E−04 | CCDC144A |
| 6249 | 237475_x_at | 8.63E−04 | CCDC152 |
| 1562 | 236900_x_at | 6.43E−04 | CCDC163P |
| 9642 | 1559003_a_at | 2.00E−04 | CCDC163P |
| 1841 | 1555443_at | 5.00E−04 | CCDC172 |
| 3020 | 1560177_at | 6.97E−05 | CCDC66 |
| 9628 | 220180_at | 1.70E−04 | CCDC68 |
| 5937 | 212886_at | 6.05E−05 | CCDC69 |
| 2045 | 228495_at | 0.00101 | CCDC75 |
| 559 | 217988_at | 2.26E−05 | CCNB1IP1 |
| 8885 | 232768_at | 3.00E−06 | CCNB2 |
| 2045 | 201974_s_at | 2.66E−04 | CCZ1 |
| 5496 | 215024_at | 0.001222 | CCZ1B |
| 6008 | 208651_x_at | 2.00E−04 | CD24 |
| 5732 | 1559583_at | 2.94E−04 | CD276 |
| 2824 | 1552509_a_at | 1.76E−04 | CD300LG |
| 9338 | 205264_at | 3.61E−04 | CD3EAP |
| 9628 | 216424_at | 2.08E−04 | CD4 |
| 5170 | 1565868_at | 5.00E−04 | CD44 |
| 2786 | 208783_s_at | 0.005 | CD46 |
| 2824 | 203507_at | 1.42E−04 | CD68 |
| 5328 | 203904_x_at | 1.26E−04 | CD82 |
| 7421 | 244352_at | 5.21E−04 | CD84 |
| 8366 | 228516_at | 1.22E−04 | CDAN1 |
| 6249 | 1564674_a_at | 4.61E−04 | CDC20B |
| 9365 | 1555346_at | 0.001444 | CDC20B |
| 3465 | 1557963_at | 2.21E−05 | CDC42BPB |
| 483 | 209057_x_at | 8.89E−04 | CDC5L |
| 5170 | 203967_at | 2.10E−04 | CDC6 |
| 5937 | 235650_at | 6.54E−04 | CDHR3 /// LOC100652903 |
| 6075 | 210965_x_at | 2.00E−06 | CDK13 |
| 10019 | 232266_x_at | 7.89E−04 | CDK13 |
| 5328 | 220039_s_at | 4.57E−05 | CDKAL1 |
| 8015 | 1561190_at | 7.57E−04 | CDKL3 |
| 5858 | 235006_at | 4.32E−04 | CDKN2AIPNL |
| 4267 | 211889_x_at | 5.00E−05 | CEACAM1 |
| 8156 | 231772_x_at | 1.89E−04 | CENPH |
| 3592 | 232065_x_at | 6.69E−04 | CENPL |
| 8224 | 232106_s_at | 0.00182 | CEP89 |
| 6251 | 223815_at | 1.04E−05 | CEP95 |
| 796 | 235918_x_at | 2.48E−04 | CEP97 |
| 5409 | 241836_x_at | 9.00E−04 | CEP97 |
| 6477 | 220335_x_at | 8.47E−05 | CES3 |
| 3548 | 1554459_s_at | 1.00E−04 | CFHR3 |
| 9338 | 211316_x_at | 8.37E−05 | CFLAR |
| 9338 | 209508_x_at | 1.87E−05 | CFLAR |
| 8306 | 243751_at | 3.60E−04 | CHD2 |
| 5328 | 231994_at | 1.88E−04 | CHDH |
| 5328 | 1559591_s_at | 2.00E−04 | CHDH |
| 2922 | 218177_at | 3.00E−04 | CHMP1B |
| 897 | 217837_s_at | 3.00E−06 | CHMP3 /// RNF103-CHMP3 |
| 3407 | 211587_x_at | 7.78E−04 | CHRNA3 |
| 5328 | 207516_at | 9.37E−05 | CHRNB4 |
| 2824 | 221164_x_at | 3.32E−04 | CHST5 |
| 2824 | 223942_x_at | 3.20E−04 | CHST5 |
| 2824 | 239053_at | 5.25E−05 | CIAO1 |
| 2824 | 217501_at | 2.74E−04 | CIAO1 |
| 5328 | 1554839_at | 2.58E−04 | CIDECP |
| 8270 | 205101_at | 0.001701 | CIITA |
| 8320 | 217598_at | 2.58E−04 | CINP |
| 285 | 219859_at | 0.00105 | CLEC4E |
| 2582 | 1555213_a_at | 7.00E−05 | CLEC7A |
| 9352 | 242287_at | 7.32E−04 | CLIP1 |
| 3964 | 1558186_s_at | 5.00E−04 | CLLU1 /// LOC100507616 |
| 5526 | 219340_s_at | 0.002 | CLN8 |
| 5328 | 219621_at | 1.23E−04 | CLSPN |
| 6846 | 220855_at | 1.00E−05 | CLTC-IT1 |
| 6381 | 234981_x_at | 0.001001 | CMBL |
| 5114 | 227522_at | 0.00102 | CMBL |
| 7567 | 1562460_at | 0.001025 | CNDP2 |
| 5170 | 217752_s_at | 5.98E−04 | CNDP2 |
| 7366 | 222959_at | 0.002 | CNGB3 |
| 5170 | 201605_x_at | 3.13E−04 | CNN2 |
| 559 | 223431_at | 1.40E−05 | CNO |
| 1043 | 206497_at | 1.60E−04 | COA1 |
| 2824 | 238863_x_at | 6.98E−05 | COG8 |
| 796 | 233109_at | 7.34E−05 | COL12A1 |
| 345 | 232638_at | 6.49E−04 | COL20A1 |
| 9002 | 221152_at | 2.87E−04 | COL8A1 |
| 8147 | 209133_s_at | 5.12E−06 | COMMD4 |
| 4267 | 1559862_at | 4.00E−06 | COPA |
| 5328 | 208684_at | 1.45E−04 | COPA |
| 1982 | 220469_at | 2.00E−05 | COPE |
| 8320 | 221550_at | 3.80E−04 | COX15 |
| 8303 | 231831_at | 1.30E−06 | COX19 |
| 492 | 242280_x_at | 4.42E−04 | CPEB4 |
| 2824 | 243403_x_at | 4.75E−05 | CPM |
| 4268 | 206100_at | 2.13E−04 | CPM |
| 6893 | 231336_at | 0.002 | CPNE4 |
| 4579 | 204920_at | 0.001492 | CPS1 |
| 7992 | 225172_at | 0.00225 | CRAMP1L |
| 5328 | 203898_at | 1.42E−04 | CRCP |
| 796 | 1569293_x_at | 7.15E−05 | CROCCP2 |
| 796 | 1569291_at | 7.15E−05 | CROCCP2 |
| 716 | 204573_at | 0.001 | CROT |
| 796 | 211286_x_at | 7.61E−05 | CSF2RA |
| 5526 | 1553871_at | 0.002 | CSNK1G2-AS1 |
| 6690 | 206075_s_at | 4.00E−05 | CSNK2A1 /// CSNK2A1P |
| 8824 | 1558493_at | 0.003 | CSTT |
| 796 | 232066_x_at | 4.36E−04 | CTBP1-AS1 |
| 5736 | 215377_at | 1.48E−04 | CTBP2 |
| 2824 | 213275_x_at | 2.90E−04 | CTSB |
| 9526 | 231234_at | 4.00E−05 | CTSC |
| 5736 | 202901_x_at | 2.92E−04 | CTSS |
| 5328 | 214782_at | 2.39E−05 | CTTN |
| 663 | 1561501_s_at | 0.003 | CTU2 |
| 416 | 206775_at | 1.49E−04 | CUBN |
| 6310 | 233568_x_at | 0.001254 | CWF19L1 |
| 5736 | 220252_x_at | 1.28E−04 | CXorf21 |
| 5328 | 228700_at | 1.61E−04 | CXorf38 |
| 5170 | 215803_at | 6.66E−05 | CXorf57 |
| 5736 | 207986_x_at | 1.87E−04 | CYB561 |
| 8303 | 201633_s_at | 0.001008 | CYB5B |
| 1047 | 1560043_at | 1.52E−04 | CYB5R1 |
| 4579 | 207780_at | 5.01E−04 | CYLC2 |
| 4579 | 217133_x_at | 3.75E−04 | CYP2B6 |
| 492 | 206755_at | 6.49E−04 | CYP2B6 |
| 2824 | 210272_at | 0.001321 | CYP2B7P1 |
| 4579 | 205998_x_at | 1.55E−04 | CYP3A4 |
| 6067 | 206515_at | 0.002 | CYP4F3 |
| 9628 | 224278_at | 3.47E−04 | CYP4F30P |
| 6698 | 216288_at | 5.08E−05 | CYSLTR1 |
| 3548 | 1559471_s_at | 3.00E−06 | D21S2088E |
| 5736 | 1562772_a_at | 2.93E−04 | DAND5 |
| 796 | 232516_x_at | 2.59E−04 | DAP3 |
| 2824 | 219290_x_at | 1.79E−04 | DAPP1 |
| 5328 | 206661_at | 8.13E−05 | DBF4B |
| 3620 | 219149_x_at | 2.76E−04 | DBR1 |
| 3465 | 205369_x_at | 2.20E−04 | DBT |
| 4793 | 244687_at | 3.59E−05 | DBT |
| 5328 | 205370_x_at | 2.18E−04 | DBT |
| 796 | 232603_at | 2.95E−04 | DCDC5 |
| 2824 | 219678_x_at | 5.78E−04 | DCLRE1C |
| 3392 | 244777_at | 4.00E−05 | DCP2 |
| 9628 | 232505_at | 1.49E−04 | DCTN2 |
| 2824 | 208674_x_at | 1.50E−04 | DDOST |
| 1890 | 216679_at | 0.002 | DDR1-AS1 |
| 5328 | 216459_x_at | 7.97E−05 | DDR1-AS1 |
| 6249 | 202577_s_at | 8.96E−04 | DDX19A |
| 5937 | 1570128_at | 2.96E−04 | DDX19A |
| 9628 | 232915_at | 2.90E−04 | DDX49 |
| 7645 | 231843_at | 9.40E−05 | DDX55 |
| 9345 | 238525_at | 1.28E−05 | DDX56 |
| 6953 | 242961_x_at | 9.00E−08 | DDX58 |
| 9628 | 223662_x_at | 0.001589 | DDX59 |
| 2824 | 207431_s_at | 8.29E−04 | DEGS1 |
| 8156 | 215058_at | 7.57E−04 | DENND5B |
| 3620 | 203277_at | 1.52E−05 | DFFA |
| 5016 | 202532_s_at | 0.002055 | DHFR |
| 8252 | 241727_x_at | 0.004 | DHFRL1 |
| 2824 | 213631_x_at | 1.35E−04 | DHODH |
| 8453 | 201789_at | 0.002 | DHRS7 |
| 3465 | 1559039_at | 2.71E−04 | DHX36 |
| 3577 | 244784_at | 0.001 | DHX57 |
| 3363 | 215541_s_at | 3.00E−06 | DIAPH1 |
| 9397 | 215529_x_at | 3.38E−05 | DIP2A |
| 1407 | 1553271_at | 0.0022 | DIP2B |
| 7009 | 1552674_at | 5.15E−05 | DIRC1 |
| 5043 | 218362_s_at | 3.97E−04 | D1S3 |
| 416 | 1565771_at | 2.63E−04 | DKFZp434E1119 |
| 10196 | 224123_at | 1.00E−04 | DKFZP434F142 |
| 416 | 1556821_x_at | 9.94E−05 | DLEU2 |
| 416 | 1556820_a_at | 9.94E−05 | DLEU2 |
| 5736 | 233056_x_at | 4.23E−04 | DLGAP4 |
| 5328 | 208386_x_at | 2.64E−05 | DMC1 |
| 5328 | 215173_at | 2.55E−05 | DNAAF1 |
| 5328 | 215840_at | 1.47E−04 | DNAH2 |
| 7214 | 216415_at | 1.00E−08 | DNAH3 |
| 5328 | 220725_x_at | 4.75E−05 | DNAH3 |
| 8994 | 1554334_a_at | 0.002 | DNAJA4 |
| 3363 | 214338_at | 0.002 | DNAJB12 |
| 8766 | 1568884_at | 3.26E−04 | DNAJC2 |
| 4247 | 234094_x_at | 0.002 | DNAJC21 |
| 8034 | 244193_at | 1.14E−04 | DNAJC22 |
| 9051 | 239065_at | 4.00E−05 | DNAJC22 |
| 5873 | 1570048_at | 1.51E−05 | DNAJC24 |
| 10019 | 1558546_at | 0.001092 | DNASE1 |
| 8312 | 241854_at | 0.002 | DNASE1 |
| 2814 | 243127_x_at | 0.001 | DNASE1 |
| 4793 | 209831_x_at | 0.00161 | DNASE2 |
| 6647 | 1560781_at | 1.78E−04 | DNHD1 |
| 4793 | 215135_at | 3.20E−04 | DNPEP |
| 2045 | 215151_at | 9.07E−04 | DOCK10 |
| 796 | 215361_at | 1.61E−04 | DOCK5 |
| 5736 | 1561276_at | 2.27E−04 | DOCK5 |
| 9352 | 213267_at | 6.03E−04 | DOPEY1 |
| 5328 | 215219_at | 6.10E−05 | DOPEY2 |
| 324 | 224196_x_at | 1.00E−04 | DPH5 |
| 324 | 219590_x_at | 1.00E−04 | DPH5 |
| 324 | 223671_x_at | 1.00E−04 | DPH5 |
| 6009 | 238012_at | 3.00E−04 | DPP7 |
| 5170 | 230018_at | 3.84E−04 | DPP9 |
| 2045 | 215102_at | 5.26E−05 | DPY19L1P1 |
| 5016 | 213391_at | 1.00E−09 | DPY19L4 |
| 3964 | 241992_at | 0.002 | DRAM1 |
| 7216 | 219512_at | 8.50E−04 | DSN1 |
| 2824 | 215810_x_at | 4.51E−04 | DST |
| 5170 | 211325_x_at | 5.70E−05 | DSTNP2 |
| 5127 | 244146_at | 9.00E−04 | DTWD1 |
| 5170 | 1565693_at | 1.04E−04 | DTYMK |
| 7815 | 1555404_a_at | 5.38E−05 | DUOXA1 |
| 9338 | 1563505_at | 8.78E−05 | DUSP16 |
| 7501 | 213848_at | 0.002 | DUSP7 |
| 4579 | 220774_at | 2.26E−04 | DYM |
| 9462 | 1556831_at | 4.00E−06 | DYNC1H1 |
| 796 | 1569843_at | 5.11E−05 | DYNC1I1 |
| 483 | 241757_x_at | 1.50E−04 | DYNC2LI1 |
| 7815 | 1562477_at | 2.78E−05 | EBF2 |
| 5736 | 211129_x_at | 3.49E−04 | EDA |
| 5736 | 211130_x_at | 2.77E−04 | EDA |
| 5736 | 211127_x_at | 2.77E−04 | EDA |
| 5736 | 226544_x_at | 1.55E−04 | EEF1E1-MUTED /// MUTED |
| 5736 | 210525_x_at | 4.14E−04 | EFCAB11 |
| 10019 | 233375_at | 1.45E−04 | EFCAB2 |
| 2958 | 233600_at | 0.00101 | EFCAB6 |
| 3297 | 208112_x_at | 5.00E−04 | EHD1 |
| 2824 | 204211_x_at | 3.66E−05 | EIF2AK2 |
| 5016 | 225164_s_at | 0.00149 | EIF2AK4 |
| 9628 | 222842_at | 9.72E−05 | EIF2C4 |
| 1407 | 242735_x_at | 6.80E−04 | ELF2 |
| 8015 | 238761_at | 2.02E−04 | ELK4 |
| 2536 | 205994_at | 5.00E−04 | ELK4 |
| 7454 | 226502_at | 2.00E−04 | ELMOD2 |
| 921 | 212394_at | 1.21E−04 | EMC1 |
| 8246 | 1569868_s_at | 0.00146 | EME2 |
| 5328 | 221980_at | 1.09E−04 | EMILIN2 |
| 790 | 1563471_at | 4.00E−05 | EPG5 |
| 345 | 211164_at | 6.11E−04 | EPHA3 |
| 9033 | 209588_at | 0.001 | EPHB2 |
| 8147 | 232896_at | 2.73E−04 | ERBB21P |
| 3465 | 215606_s_at | 4.83E−05 | ERC1 |
| 1043 | 208247_at | 5.27E−04 | ERC2-IT1 |
| 5328 | 224024_at | 7.68E−05 | ERG1C1 |
| 1043 | 214372_x_at | 4.51E−04 | ERN2 |
| 3620 | 1562777_at | 5.03E−05 | ERV3-1 |
| 3509 | 215594_at | 4.00E−05 | ERV9-1 |
| 8900 | 1555753_x_at | 5.00E−04 | ERVH-6 |
| 5053 | 1555299_s_at | 4.00E−10 | ERVW-1 |
| 5053 | 1553352_x_at | 4.00E−10 | ERVW-1 |
| 9628 | 1564473_at | 1.72E−04 | ESCO2 |
| 6358 | 211627_x_at | 4.00E−08 | ESR1 |
| 796 | 1569554_at | 2.43E−05 | ESR2 |
| 5736 | 220838_at | 9.22E−05 | EXD3 |
| 9338 | 1559044_at | 1.42E−04 | EXOSC1 |
| 9475 | 209527_at | 7.85E−05 | EXOSC2 |
| 4972 | 1556436_at | 2.04E−04 | EXTL3-AS1 |
| 5316 | 203989_x_at | 2.07E−04 | F2R |
| 5873 | 206795_at | 1.71E−04 | F2RL2 |
| 504 | 204713_s_at | 3.00E−06 | F5 |
| 5328 | 1566123_at | 1.86E−05 | FABP6 |
| 2824 | 208963_x_at | 4.33E−05 | FADS1 /// MIR1908 |
| 2536 | 212106_at | 5.00E−04 | FAF2 |
| 796 | 1558976_x_at | 2.52E−04 | FAM100A |
| 796 | 1558975_at | 2.52E−04 | FAM100A |
| 4814 | 220575_at | 5.44E−04 | FAM106A |
| 7645 | 227551_at | 2.55E−04 | FAM108B1 |
| 8324 | 204403_x_at | 7.48E−04 | FAM115A /// LOC100294033 |
| 4386 | 221233_s_at | 0.002 | FAM135A |
| 2188 | 213458_at | 6.00E−05 | FAM149B1 |
| 3588 | 1555379_at | 1.12E−04 | FAM159A |
| 5526 | 222929_at | 4.50E−05 | FAM160B2 |
| 5526 | 222928_s_at | 4.50E−05 | FAM160B2 |
| 345 | 242608_x_at | 2.22E−04 | FAM161B |
| 2339 | 1553909_x_at | 0.001667 | FAM178A |
| 2667 | 1563638_at | 2.00E−04 | FAM18A |
| 5328 | 242945_at | 1.21E−04 | FAM20A |
| 5016 | 230739_at | 0.001623 | FAM210A |
| 10019 | 1553811_at | 5.84E−04 | FAM222A-AS1 |
| 9345 | 216786_at | 2.85E−04 | FAM224A |
| 8699 | 231880_at | 0.00194 | FAM40B |
| 5736 | 217535_at | 2.34E−04 | FAM49B |
| 5736 | 217534_at | 2.40E−04 | FAM49B |
| 9338 | 226062_x_at | 3.67E−04 | FAM63A |
| 4972 | 222194_at | 1.66E−04 | FAM66D |
| 7605 | 1568891_x_at | 0.002 | FANCD2 |
| 7605 | 1568889_at | 0.002 | FANCD2 |
| 123 | 220255_at | 4.13E−04 | FANCE |
| 3548 | 231971_at | 1.00E−05 | FANCM |
| 9365 | 220896_at | 7.12E−04 | FBXL18 |
| 4331 | 1553361_x_at | 0.00182 | FBXL18 |
| 4331 | 1553359_at | 0.00182 | FBXL18 |
| 7191 | 237036_at | 1.70E−06 | FBXO10 |
| 8303 | 220233_at | 5.51E−04 | FBXO17 /// SARS2 |
| 9620 | 1559566_at | 8.00E−06 | FBXO42 |
| 4793 | 212991_at | 6.02E−05 | FBXO9 |
| 6774 | 1564138_at | 0.001 | FBXW12 |
| 5328 | 215600_x_at | 2.91E−05 | FBXW12 |
| 5170 | 231883_at | 3.71E−04 | FBXW8 |
| 115 | 211305_x_at | 1.54E−04 | FCAR |
| 5937 | 241954_at | 5.86E−04 | FDFT1 |
| 363 | 239161_at | 4.00E−04 | FDX1 |
| 7547 | 213341_at | 1.00E−05 | FEM1C |
| 9338 | 205973_at | 3.45E−04 | FEZ1 |
| 5328 | 215602_at | 5.74E−05 | FGD2 |
| 8766 | 207822_at | 2.05E−04 | FGFR1 |
| 2824 | 215404_x_at | 1.80E−04 | FGFR1 |
| 5736 | 214124_x_at | 2.33E−04 | FGFR1OP |
| 3465 | 232186_at | 5.29E−04 | FITM2 |
| 2113 | 224284_x_at | 8.97E−06 | FKSG49 |
| 2113 | 224288_x_at | 6.77E−05 | FKSG49 |
| 186 | 211454_x_at | 2.00E−07 | FKSG49 |
| 3822 | 208120_x_at | 1.33E−04 | FKSG49 /// LOC100506290 /// LOC100653153 |
| 7645 | 1552664_at | 1.71E−04 | FLCN |
| 8269 | 215645_at | 6.74E−04 | FLCN |
| 3465 | 217342_x_at | 1.14E−04 | FLJ11292 |
| 1562 | 220529_at | 7.42E−04 | FLJ11710 |
| 5736 | 233210_at | 5.22E−05 | FLJ12120 |
| 7300 | 1570414_x_at | 2.08E−05 | FLJ13197 |
| 9345 | 1559011_at | 1.19E−05 | FLJ13773 |
| 416 | 220401_at | 0.001067 | FLJ21369 |
| 8355 | 234216_at | 0.002 | FLJ21408 |
| 1043 | 234627_at | 9.81E−05 | FLJ21408 |
| 559 | 1553258_at | 2.76E−05 | FLJ30679 |
| 5736 | 1557472_a_at | 4.48E−04 | FLJ30838 |
| 5736 | 1561099_at | 2.35E−04 | FLJ32756 |
| 2824 | 235628_x_at | 2.17E−04 | FLJ33630 |
| 2824 | 1566760_at | 1.80E−05 | FLJ34208 |
| 2045 | 1564208_x_at | 3.51E−04 | FLJ35390 |
| 5043 | 1569090_x_at | 5.95E−04 | FLJ35390 |
| 2045 | 1569089_a_at | 8.02E−04 | FLJ35390 /// LOC100506683 |
| 3952 | 1559277_at | 1.00E−08 | FLJ35700 |
| 1439 | 1564413_at | 7.30E−04 | FLJ36116 |
| 932 | 231882_at | 9.75E−05 | FLJ39632 |
| 796 | 1553145_at | 1.00E−04 | FLJ39653 |
| 5328 | 220352_x_at | 2.25E−04 | FLJ42627 |
| 4253 | 227129_x_at | 0.002 | FLJ45340 |
| 719 | 225899_x_at | 5.50E−05 | FLJ45340 /// FLJ45445 /// LOC100128326 /// LOC100132050 /// |
| | | | LOC100287894 /// LOC100289306 /// LOC100506479 /// LOC100508632 |
| | | | /// LOC100652945 /// LOC100653241 /// LOC100653346 /// LOC729737 |
| 6251 | 1565786_x_at | 4.89E−05 | FLJ45482 |
| 2045 | 1558148_x_at | 0.00168 | FLJ90757 |
| 5309 | 1566558_x_at | 5.00E−04 | FLJ90757 |
| 5016 | 1569688_at | 0.004 | FMO5 |
| 5496 | 1558199_at | 0.001243 | FN1 |
| 2824 | 242472_x_at | 2.05E−04 | FNBP4 |
| 5170 | 235101_at | 0.001185 | FNBP4 |
| 2045 | 230808_at | 6.11E−04 | FNTA |
| 5016 | 1555305_at | 0.001241 | FOXJ2 |
| 1717 | 232096_x_at | 1.00E−05 | FOXP1-IT1 |
| 10019 | 233106_at | 1.57E−04 | FRMD6-AS1 |
| 7984 | 1562650_at | 3.20E−04 | FRYL |
| 1046 | 244419_at | 0.00105 | FRZB |
| 8147 | 233741_at | 1.86E−04 | FTCD |
| 5016 | 231108_at | 0.001515 | FUS |
| 7421 | 210608_s_at | 0.002056 | FUT2 |
| 796 | 214088_s_at | 1.34E−05 | FUT3 |
| 9020 | 1565661_x_at | 1.00E−04 | FUT6 |
| 9020 | 1565660_at | 1.00E−04 | FUT6 |
| 8058 | 209415_at | 0.002 | FZR1 |
| 8892 | 223255_at | 0.001765 | G2E3 |
| 1129 | 1557350_at | 7.55E−04 | G3BP1 |
| 9397 | 222187_x_at | 2.59E−04 | G3BP1 |
| 9338 | 206952_at | 1.59E−04 | G6PC |
| 8386 | 202812_at | 1.00E−05 | GAA |
| 4579 | 219815_at | 4.72E−04 | GAL3ST4 |
| 9731 | 203398_s_at | 2.00E−05 | GALNT3 |
| 5170 | 219956_at | 4.53E−04 | GALNT6 |
| 5115 | 219110_at | 0.002 | GAR1 |
| 559 | 210005_at | 6.50E−04 | GART |
| 559 | 224841_x_at | 0.0025 | GAS5 /// SNORD44 /// SNORD47 /// SNORD76 /// SNORD77 /// |
| | | | SNORD79 /// SNORD80 /// SNORD81 |
| 559 | 224741_x_at | 0.0025 | GAS5 /// SNORD44 /// SNORD47 /// SNORD76 /// SNORD77 /// |
| | | | SNORD79 /// SNORD80 /// SNORD81 |
| 8830 | 214718_at | 0.003 | GATAD1 |
| 7678 | 216733_s_at | 3.00E−06 | GATM |
| 6515 | 235175_at | 5.89E−05 | GBP4 |
| 2824 | 1560227_at | 7.32E−05 | GDPD1 |
| 7944 | 1554670_at | 4.00E−05 | GGA1 |
| 9365 | 1554419_x_at | 6.90E−04 | GGNBP2 |
| 8303 | 233837_at | 4.32E−04 | GGT1 |
| 559 | 237052_x_at | 0.00167 | GIGYF2 |
| 5043 | 1552318_at | 1.41E−04 | GIMAP1 |
| 9628 | 240778_at | 2.29E−04 | GINS4 |
| 6878 | 234116_at | 7.00E−04 | GJD4 |
| 3646 | 1553772_at | 1.09E−05 | GK5 |
| 483 | 214205_x_at | 6.85E−04 | GLRX3 |
| 9338 | 234918_at | 3.68E−04 | GLTSCR2 |
| 6675 | 232703_at | 2.15E−05 | GLUD1P7 |
| 9475 | 223563_at | 5.18E−04 | GNB1L |
| 2110 | 223487_x_at | 6.36E−04 | GNB4 |
| 7564 | 232043_at | 2.66E−06 | GNG7 |
| 6173 | 233544_at | 8.38E−04 | GNL1 |
| 7645 | 222109_at | 6.54E−04 | GNL3L |
| 5170 | 212959_s_at | 3.20E−04 | GNPTAB |
| 4268 | 215203_at | 9.48E−06 | GOLGA4 |
| 1043 | 1561170_at | 1.03E−05 | GOLGA6L1 /// GOLGA6L6 /// LOC440243 |
| 9395 | 208797_s_at | 5.00E−06 | GOLGA8A |
| 3465 | 213650_at | 2.00E−04 | GOLGA8A /// GOLGA8B /// LOC100508892 |
| 6249 | 217771_at | 0.001828 | GOLM1 |
| 9345 | 219846_at | 2.83E−04 | GON4L |
| 8453 | 213020_at | 1.00E−05 | GOSR1 |
| 2824 | 206681_x_at | 1.19E−04 | GP2 |
| 6442 | 206264_at | 0.0018 | GPLD1 |
| 5328 | 214605_x_at | 1.04E−04 | GPR1 |
| 5016 | 223887_at | 0.001934 | GPR132 |
| 9345 | 220901_at | 1.82E−04 | GPR157 |
| 9573 | 232350_x_at | 2.18E−04 | GPR161 |
| 9345 | 224275_at | 3.14E−04 | GPR98 |
| 3822 | 219327_s_at | 0.002 | GPRC5C |
| 2824 | 228141_at | 2.94E−04 | GPX8 |
| 3402 | 227628_at | 1.58E−04 | GPX8 |
| 264 | 1560265_at | 1.55E−04 | GRIK2 |
| 5170 | 1555375_at | 3.27E−04 | GRIK2 |
| 9345 | 1560543_at | 2.12E−05 | GRK4 |
| 6249 | 223759_s_at | 0.00108 | GSG2 |
| 1129 | 1564525_at | 1.37E−05 | GSN |
| 8991 | 217595_at | 8.31E−04 | GSPT1 |
| 9020 | 232193_at | 0.001 | GSTT1 |
| 144 | 230376_at | 2.70E−04 | GTF2A2 |
| 8702 | 202355_s_at | 0.001 | GTF2F1 |
| 5328 | 221540_x_at | 2.23E−05 | GTF2H2 /// GTF2H2B /// GTF2H2C /// GTF2H2D |
| 5937 | 222104_x_at | 8.13E−04 | GTF2H3 |
| 5736 | 232905_at | 2.91E−04 | GTF2H5 |
| 5764 | 210891_s_at | 0.003 | GTF2I /// GTF2IP1 /// LOC100093631 |
| 5328 | 1557289_s_at | 2.93E−04 | GTF2IRD2 /// GTF2IRD2B |
| 5736 | 213835_x_at | 3.10E−04 | GTPBP3 |
| 123 | 211040_x_at | 6.40E−04 | GTSE1 |
| 123 | 204318_s_at | 6.75E−04 | GTSE1 |
| 123 | 215942_s_at | 8.00E−04 | GTSE1 |
| 2824 | 1556742_at | 4.42E−04 | GUSBP1 |
| 5736 | 215202_at | 1.53E−04 | GUSBP11 |
| 7567 | 239924_at | 2.83E−07 | GUSBP11 |
| 9345 | 215599_at | 1.92E−04 | GUSBP3 /// GUSBP9 |
| 9338 | 1555685_at | 2.69E−04 | H6PD |
| 5937 | 216875_x_at | 2.83E−04 | HAB1 |
| 9338 | 220071_x_at | 1.69E−04 | HAUS2 |
| 1732 | 1555629_at | 2.10E−05 | HAVCR2 |
| 2824 | 1569503_at | 1.25E−04 | HEATR5B |
| 5937 | 1570200_at | 6.58E−04 | HELB |
| 9023 | 220085_at | 5.00E−07 | HELLS |
| 8453 | 241784_x_at | 1.00E−06 | HELQ |
| 345 | 233075_at | 0.001026 | HERC2P7 |
| 2129 | 219352_at | 0.002 | HERC6 |
| 796 | 211328_x_at | 3.65E−04 | HFE |
| 2824 | 232627_at | 4.29E−05 | HGS |
| 3396 | 230135_at | 0.001 | HHIP |
| 6385 | 220812_s_at | 0.001201 | HHLA2 |
| 6515 | 220387_s_at | 5.01E−05 | HHLA3 |
| 6515 | 234703_at | 5.01E−05 | HHLA3 |
| 6515 | 234665_x_at | 5.01E−05 | HHLA3 |
| 3465 | 1555318_at | 4.64E−05 | HIF3A |
| 6260 | 226364_at | 0.005 | HIP1 |
| 2300 | 219028_at | 0.002 | H1PK2 |
| 5016 | 1569980_x_at | 5.13E−04 | HKR1 |
| 5016 | 1569979_at | 5.13E−04 | HKR1 |
| 5736 | 208306_x_at | 6.16E−05 | HLA-DRB1 /// LOC100507709 /// LOC100507714 |
| 2824 | 200905_x_at | 8.54E−04 | HLA-E |
| 4808 | 217456_x_at | 5.94E−05 | HLA-E |
| 194 | 1562381_at | 3.33E−06 | HLA-F-AS1 |
| 1047 | 242649_x_at | 5.79E−04 | HMGN2P46 |
| 2110 | 212109_at | 9.03E−04 | HN1L |
| 9630 | 211732_x_at | 6.69E−04 | HNMT |
| 7501 | 206809_s_at | 0.002 | HNRNPA3 /// HNRNPA3P1 |
| 7501 | 206808_at | 0.002 | HNRNPA3P1 |
| 7021 | 227110_at | 0.002 | HNRNPC /// LOC100652761 /// LOC100653343 |
| 5382 | 200775_s_at | 5.50E−05 | HNRNPK /// MIR7-1 |
| 796 | 233605_x_at | 4.87E−05 | HNRNPM |
| 6249 | 1558315_s_at | 5.26E−04 | HOOK3 |
| 5328 | 1566140_at | 7.49E−05 | HOPX |
| 5889 | 229400_at | 0.0011 | HOXD10 |
| 5736 | 224592_x_at | 6.05E−04 | HP1BP3 |
| 2045 | 204544_at | 4.95E−04 | HPS5 |
| 3204 | 222881_at | 1.00E−05 | HPSE |
| 6105 | 210013_at | 2.00E−17 | HPX |
| 2300 | 212983_at | 0.002 | HRAS |
| 9628 | 221170_at | 1.10E−04 | HRH4 |
| 1043 | 223647_x_at | 9.77E−05 | HSCB |
| 7645 | 220081_x_at | 4.95E−04 | HSD17B7 |
| 123 | 223248_at | 4.44E−04 | HSDL1 |
| 4972 | 215436_at | 2.63E−04 | HSDL2 |
| 5652 | 207135_at | 7.00E−05 | HTR2A |
| 5935 | 1553706_at | 6.25E−04 | HTRA4 |
| 1043 | 217618_x_at | 1.53E−04 | HUS1 |
| 2824 | 1563529_at | 5.30E−05 | HYDIN2 |
| 10019 | 215490_at | 7.19E−04 | IBA57 |
| 7472 | 211740_at | 0.002 | ICA1 |
| 5328 | 207194_s_at | 5.62E−05 | ICAM4 |
| 7564 | 236400_at | 5.03E−04 | IDH1-AS1 |
| 5736 | 212221_x_at | 1.87E−04 | IDS |
| 6070 | 212203_x_at | 1.00E−05 | IFITM3 |
| 5328 | 232844_at | 2.52E−04 | IFT140 |
| 5679 | 223736_at | 5.24E−04 | IFT81 |
| 5937 | 1569033_at | 3.82E−04 | IGF2BP3 |
| 7815 | 230128_at | 4.00E−05 | IGLL5 |
| 5937 | 235202_x_at | 8.04E−04 | IKBIP |
| 1043 | 207433_at | 1.03E−04 | IL10 |
| 3465 | 206924_at | 2.07E−04 | IL11 |
| 8324 | 1552584_at | 2.45E−04 | IL12RB1 |
| 3465 | 206295_at | 2.18E−04 | IL18 |
| 796 | 208200_at | 1.50E−05 | IL1A |
| 8015 | 205945_at | 3.00E−04 | IL6R |
| 9378 | 218637_at | 0.002 | IMPACT |
| 8463 | 223871_x_at | 2.55E−04 | ING5 |
| 4793 | 1560558_at | 1.52E−04 | INIP |
| 439 | 221251_x_at | 0.004 | INO80B /// INO80B-WBP1 |
| 2824 | 1559716_at | 8.97E−05 | INO80C |
| 1407 | 203607_at | 3.28E−04 | INPP5F |
| 6249 | 1560172_at | 4.23E−04 | INTS10 |
| 4787 | 237504_at | 1.00E−05 | INTS10 |
| 5736 | 233193_x_at | 3.12E−04 | INTS4 |
| 8453 | 236906_x_at | 1.00E−06 | IPO5 |
| 7313 | 244703_x_at | 3.01E−04 | IPO9 |
| 5736 | 219618_at | 1.23E−04 | IRAK4 |
| 1047 | 214666_x_at | 7.55E−05 | IREB2 |
| 8015 | 239177_at | 9.90E−04 | IRGQ |
| 663 | 216502_at | 0.001342 | ISG20L2 |
| 416 | 213475_s_at | 6.07E−04 | ITGAL |
| 720 | 216261_at | 1.55E−04 | ITGB3 |
| 5043 | 1553243_at | 1.58E−04 | ITIH5 |
| 5170 | 216958_s_at | 8.40E−05 | IVD |
| 7472 | 238600_at | 0.0011 | JAKMIP1 |
| 5328 | 215233_at | 1.87E−04 | JMJD6 |
| 5016 | 232756_at | 0.001131 | KALRN |
| 5889 | 218418_s_at | 0.00135 | KANK2 |
| 7645 | 1559142_at | 2.56E−04 | KAT6A |
| 5328 | 1562236_at | 3.18E−04 | KAT6B |
| 6550 | 233649_at | 0.0015 | KATNAL2 |
| 6953 | 233644_at | 5.00E−04 | KATNAL2 |
| 5679 | 1554710_at | 7.85E−04 | KCNMB1 |
| 559 | 1557621_at | 1.56E−04 | KCP |
| 6249 | 1553136_at | 2.84E−04 | KCTD18 |
| 5114 | 228299_at | 0.002 | KCTD20 |
| 5328 | 223176_at | 1.41E−05 | KCTD20 |
| 559 | 1553717_at | 1.52E−04 | KCTD7 /// RABGEF1 |
| 7984 | 1553150_at | 6.11E−04 | KDM1B |
| 4793 | 222359_x_at | 2.34E−04 | KDSR |
| 5328 | 202714_s_at | 4.94E−05 | KIAA0391 |
| 796 | 205496_at | 6.88E−06 | KIAA0408 |
| 483 | 233915_at | 3.00E−04 | KIAA0825 |
| 3548 | 232244_at | 1.00E−05 | KIAA1161 |
| 6251 | 231856_at | 2.37E−04 | KIAA1244 |
| 123 | 232250_at | 1.14E−04 | KIAA1257 |
| 8355 | 231887_s_at | 4.00E−05 | KIAA1274 |
| 7944 | 236325_at | 0.002 | KIAA1377 |
| 8366 | 232166_at | 3.02E−04 | KIAA1377 |
| 2584 | 1554550_at | 5.00E−04 | KIAA1430 |
| 4579 | 230567_at | 8.14E−05 | KIAA1430 |
| 5016 | 213316_at | 4.00E−04 | KIAA1462 |
| 4906 | 232921_at | 2.55E−04 | KIAA1549 |
| 4888 | 221802_s_at | 0.003 | KIAA1598 |
| 6217 | 1569365_a_at | 4.00E−06 | KIAA1656 |
| 3660 | 215018_at | 0.002 | KIAA1731 |
| 4082 | 231893_at | 0.002 | KIAA1755 |
| 2110 | 243539_at | 4.97E−04 | KIAA1841 |
| 4793 | 209245_s_at | 8.11E−05 | KIF1C |
| 2824 | 211351_at | 6.27E−05 | KIF25-AS1 |
| 9628 | 203943_at | 6.25E−04 | KIF3B |
| 8314 | 216428_x_at | 5.08E−05 | KIR3DX1 |
| 5736 | 244276_at | 3.03E−04 | KLB |
| 492 | 206551_x_at | 3.08E−04 | KLHL24 |
| 5328 | 220374_at | 7.21E−05 | KLHL28 |
| 2765 | 238523_at | 2.00E−04 | KLHL36 |
| 4808 | 1560396_at | 3.38E−04 | KLHL6 |
| 8855 | 233854_x_at | 1.77E−05 | KLK4 |
| 8855 | 224062_x_at | 1.77E−05 | KLK4 |
| 1604 | 207796_x_at | 5.00E−04 | KLRD1 |
| 1738 | 216713_at | 6.91E−04 | KRIT1 |
| 2108 | 204738_s_at | 4.00E−06 | KRIT1 |
| 5328 | 232441_at | 1.65E−04 | KRR1 |
| 9276 | 237853_x_at | 4.67E−04 | KRTAP10-12 |
| 8240 | 234880_x_at | 6.73E−05 | KRTAP1-3 |
| 8242 | 233631_x_at | 1.93E−06 | KRTAP9-2 /// KRTAP9-8 |
| 559 | 233122_at | 2.38E−04 | KRTCAP2 |
| 1043 | 220089_at | 6.16E−04 | L2HGDH |
| 5736 | 217810_x_at | 1.86E−04 | LARS |
| 1043 | 211768_at | 0.001113 | LAT2 |
| 5944 | 207734_at | 6.70E−04 | LAX1 |
| 9475 | 1553888_at | 5.64E−04 | LDHAL6A |
| 194 | 202068_s_at | 1.00E−04 | LDLR |
| 7472 | 244338_at | 0.0011 | LDLRAD3 |
| 1444 | 224673_at | 0.001929 | LENG8 |
| 1444 | 1554589_at | 0.001929 | LENG9 |
| 796 | 207092_at | 4.55E−06 | LEP |
| 2824 | 222006_at | 4.58E−05 | LETM1 |
| 2824 | 1557197_a_at | 4.68E−04 | LGALS3 |
| 1732 | 232457_at | 2.54E−04 | LIMCH1 |
| 4579 | 222762_x_at | 8.70E−05 | LIMD1 |
| 5043 | 1566129_at | 2.33E−04 | LIMS1 |
| 796 | 231959_at | 1.85E−05 | LIN52 |
| 5736 | 223350_x_at | 1.38E−04 | LIN7C |
| 9628 | 1562733_at | 2.95E−04 | LINC00092 |
| 7633 | 1562802_at | 1.33E−04 | LINC00210 |
| 4793 | 210711_at | 5.25E−05 | LINC00260 |
| 614 | 1569755_at | 0.0015 | LINC00276 |
| 2824 | 221621_at | 1.23E−04 | LINC00338 /// SCARNA16 |
| 2824 | 1552979_at | 2.82E−04 | LINC00471 |
| 8147 | 1553484_at | 4.69E−05 | LINC00477 |
| 7645 | 1556072_at | 0.002 | LINC00528 |
| 416 | 232034_at | 9.52E−05 | LINC00537 |
| 5328 | 1569983_at | 2.72E−05 | LINC00566 |
| 2824 | 220904_at | 7.33E−05 | LINC00574 |
| 4331 | 1560419_at | 0.00105 | LOC100127974 |
| 7992 | 1565723_at | 3.45E−04 | LOC100128281 |
| 3465 | 233527_at | 0.00138 | LOC100129069 |
| 10019 | 244730_x_at | 7.83E−05 | LOC100129195 |
| 4808 | 221477_s_at | 2.07E−04 | LOC100129518 /// SOD2 |
| 8378 | 1559528_at | 1.00E−04 | LOC100129917 |
| 5618 | 241851_x_at | 2.16E−05 | LOC100130429 |
| 5328 | 1564021_at | 1.46E−04 | LOC100130456 |
| 9628 | 1564240_at | 2.79E−04 | LOC100130856 |
| 5736 | 233840_at | 6.16E−04 | LOC100130950 |
| 8699 | 242364_x_at | 0.00115 | LOC100131096 |
| 7645 | 1558152_at | 3.12E−04 | LOC100131262 |
| 9628 | 1568864_at | 1.51E−04 | LOC100131691 |
| 5526 | 1559180_at | 1.00E−05 | LOC100131860 |
| 6861 | 1564660_at | 1.00E−05 | LOC100131864 |
| 4253 | 234664_at | 0.001002 | LOC100132062 /// LOC100288102 /// LOC100506479 /// LOC731275 |
| 5937 | 1558527_at | 5.59E−05 | LOC100132707 |
| 8768 | 227234_at | 1.00E−06 | LOC100132815 |
| 4255 | 1565748_at | 1.13E−04 | LOC100132815 |
| 9872 | 1565746_at | 1.00E−04 | LOC100132815 |
| 7481 | 1561482_at | 0.005 | LOC100133308 |
| 5328 | 232190_x_at | 3.87E−05 | LOC100133445 /// LOC115110 |
| 5328 | 1569604_at | 1.55E−04 | LOC100133612 |
| 1353 | 232579_at | 2.63E−05 | LOC100134229 |
| 5865 | 229930_at | 0.003 | LOC100134361 |
| 4331 | 1569270_at | 1.95E−04 | LOC100134368 |
| 5736 | 231886_at | 8.39E−05 | LOC100134822 |
| 1732 | 211050_x_at | 6.15E−04 | LOC100134822 /// LOC100288069 /// LOC100653347 |
| 5736 | 232731_x_at | 5.89E−05 | LOC100190938 |
| 2824 | 233174_at | 1.23E−04 | LOC100287015 |
| 9475 | 240617_at | 1.39E−04 | LOC100287098 |
| 6385 | 233997_at | 0.001456 | LOC100287166 |
| 2136 | 217220_at | 6.81E−04 | LOC100287387 |
| 9381 | 230606_at | 7.00E−05 | LOC100287525 |
| 8885 | 1569446_x_at | 4.00E−05 | LOC100287576 /// LOC100506454 |
| 2824 | 214871_x_at | 4.19E−04 | LOC100287590 |
| 5736 | 1562095_at | 3.38E−04 | LOC100287789 /// SLMAP |
| 7908 | 214870_x_at | 6.67E−13 | LOC100288332 /// LOC100506193 /// LOC642778 /// LOC642799 /// |
| | | | NPIP /// PKD1P1 |
| 7908 | 221501_x_at | 6.67E−13 | LOC100288332 /// LOC642778 /// LOC642799 /// PKD1P1 |
| 5736 | 233775_x_at | 1.33E−04 | LOC100289333 |
| 5170 | 232420_x_at | 4.74E−04 | LOC100289341 |
| 9515 | 1564639_at | 2.02E−05 | LOC100291323 |
| 3407 | 1569378_at | 2.41E−05 | LOC100292680 |
| 932 | 214809_at | 2.76E−04 | LOC100294145 |
| 5328 | 1569078_at | 5.58E−05 | LOC100294362 |
| 6493 | 222947_at | 4.00E−07 | LOC100379224 |
| 3426 | 1564463_at | 2.67E−04 | LOC100499221 |
| 663 | 238901_at | 4.36E−04 | LOC100505633 |
| 2824 | 1557068_at | 1.14E−04 | LOC100505782 |
| 3212 | 1558906_a_at | 2.70E−04 | LOC100505783 |
| 5736 | 1569004_at | 2.76E−04 | LOC100505812 |
| 7988 | 233307_x_at | 5.30E−04 | LOC100505828 /// MGST3 |
| 9628 | 1557004_at | 2.73E−05 | LOC100505902 /// LOC285389 |
| 4268 | 232790_at | 0.002033 | LOC100506054 |
| 7567 | 229296_at | 1.29E−04 | LOC100506119 |
| 6429 | 202477_s_at | 0.002 | LOC100506167 /// TUBGCP2 |
| 2824 | 217625_x_at | 5.15E−04 | LOC100506190 |
| 1211 | 235282_at | 1.00E−06 | LOC100506295 |
| 483 | 1560679_at | 5.40E−05 | LOC100506328 |
| 8355 | 244366_at | 4.00E−05 | LOC100506371 |
| 9338 | 232804_at | 1.60E−04 | LOC100506385 |
| 8303 | 1570350_at | 1.04E−06 | LOC100506403 /// RUNX1 |
| 3465 | 242358_at | 6.42E−05 | LOC100506451 |
| 1043 | 1564232_at | 2.72E−04 | LOC100506453 /// LOC441528 |
| 416 | 222280_at | 1.56E−04 | LOC100506469 |
| 10019 | 224766_at | 6.56E−04 | LOC100506548 /// RPL37 |
| 9475 | 1556235_at | 5.28E−04 | LOC100506560 /// LOC100509195 |
| 2584 | 232070_at | 0.002 | LOC100506639 |
| 3577 | 237844_at | 0.001 | LOC100506639 /// ZNF131 |
| 6599 | 232822_x_at | 5.00E−04 | LOC100506757 |
| 1562 | 1568722_at | 6.63E−04 | LOC100506832 |
| 4621 | 232131_at | 2.78E−04 | LOC100506874 |
| 5328 | 243924_at | 2.17E−05 | LOC100506930 |
| 1968 | 1559546_s_at | 1.00E−05 | LOC100506948 /// SNORD107 /// SNORD115-13 /// SNORD115-26 /// |
| | | | SNORD115-7 /// SNORD116-28 /// SNRPN |
| 5873 | 1559169_at | 2.64E−05 | LOC100507013 |
| 796 | 1566152_a_at | 1.67E−04 | LOC100507106 |
| 1241 | 1563776_at | 3.00E−06 | LOC100507283 |
| 1094 | 229189_s_at | 3.00E−16 | LOC100507376 |
| 1094 | 229190_at | 3.00E−16 | LOC100507376 |
| 6953 | 224006_at | 5.05E−06 | LOC100507377 |
| 6762 | 210553_x_at | 0.002 | LOC100507472 /// PCSK6 |
| 2824 | 235617_x_at | 4.19E−05 | LOC100507537 |
| 1047 | 204861_s_at | 6.32E−04 | LOC100509323 /// NAIP |
| 3588 | 232502_at | 8.51E−04 | LOC100652735 /// LOC100653122 /// LOC728558 |
| 3465 | 239168_at | 4.91E−05 | LOC100652811 /// LOC100653083 /// LOC150622 /// LOC400940 |
| 559 | 222499_at | 7.89E−04 | LOC100652993 /// LOC100653136 /// MRPS16 |
| 6249 | 220983_s_at | 4.51E−04 | LOC100653017 /// MIR612 /// NEAT1 |
| 4331 | 225239_at | 1.76E−04 | LOC100653017 /// MIR612 /// NEAT1 |
| 8303 | 215709_at | 1.60E−06 | LOC100653079 /// PRIM2 |
| 4813 | 242904_x_at | 7.00E−07 | LOC100653229 |
| 7351 | 1558202_at | 1.00E−05 | LOC145783 |
| 3592 | 235014_at | 3.93E−04 | LOC147727 |
| 5937 | 1558577_at | 2.81E−04 | LOC148709 |
| 3465 | 1561059_a_at | 3.87E−04 | LOC152024 |
| 4793 | 1562048_at | 6.73E−06 | LOC152225 |
| 8246 | 232645_at | 4.80E−04 | LOC153684 |
| 4620 | 1556224_a_at | 1.00E−10 | LOC155060 /// ZNF783 |
| 4267 | 1563827_at | 3.28E−04 | LOC158434 |
| 1353 | 1556898_at | 2.78E−05 | LOC200830 |
| 5170 | 235587_at | 1.04E−04 | LOC202781 |
| 9035 | 232294_at | 1.00E−05 | LOC219347 |
| 378 | 1569722_s_at | 0.0013 | LOC219731 |
| 2824 | 233013_x_at | 1.57E−05 | LOC220906 |
| 6953 | 232772_at | 9.00E−08 | LOC221272 |
| 9343 | 241249_at | 0.002 | LOC253039 |
| 807 | 1563074_at | 1.00E−12 | LOC255654 |
| 7009 | 215302_at | 5.33E−06 | LOC257152 |
| 2584 | 1556319_at | 0.001002 | LOC283270 |
| 2824 | 1566734_at | 2.63E−04 | LOC283454 |
| 7472 | 1561442_at | 0.0011 | LOC283585 |
| 1958 | 1561518_at | 2.50E−07 | LOC283914 |
| 2824 | 1558723_at | 8.51E−05 | LOC284014 |
| 559 | 1569006_at | 1.13E−04 | LOC284379 |
| 8156 | 1560390_s_at | 0.0021 | LOC284440 |
| 3402 | 1569318_at | 8.02E−04 | LOC284440 |
| 3592 | 1555363_s_at | 2.35E−04 | LOC284440 |
| 2045 | 235317_at | 5.25E−05 | LOC284454 |
| 416 | 1562051_at | 1.00E−04 | LOC284757 /// LOC729296 |
| 5736 | 1563680_at | 2.59E−04 | LOC284950 |
| 9040 | 1563691_at | 0.001 | LOC285300 |
| 2739 | 1569781_at | 5.00E−04 | LOC285423 |
| 9338 | 1563571_at | 1.88E−04 | LOC285463 |
| 123 | 1561566_at | 2.53E−04 | LOC285501 |
| 6188 | 1559479_at | 5.00E−04 | LOC285540 |
| 1177 | 1563489_at | 1.15E−05 | LOC285638 |
| 8453 | 1553335_x_at | 4.00E−06 | LOC285696 |
| 7547 | 234147_at | 5.00E−04 | LOC286059 |
| 6249 | 1562365_at | 2.57E−04 | LOC286177 |
| 1792 | 1560202_at | 1.00E−06 | LOC338739 |
| 6173 | 1564360_a_at | 2.68E−05 | LOC339260 |
| 483 | 227940_at | 8.19E−04 | LOC339803 |
| 416 | 232455_x_at | 3.24E−04 | LOC340085 |
| 5328 | 1560119_at | 4.70E−05 | LOC389634 |
| 559 | 222196_at | 1.73E−05 | LOC389906 |
| 7908 | 214035_x_at | 0.001 | LOC399491 |
| 133 | 1558075_at | 1.49E−05 | LOC399491 |
| 8252 | 232965_at | 0.004 | LOC400684 |
| 492 | 1569213_at | 6.35E−04 | LOC400891 |
| 7472 | 1570006_at | 6.00E−04 | LOC400958 |
| 115 | 1556609_at | 0.001274 | LOC401098 |
| 6846 | 241972_at | 1.00E−05 | LOC401588 |
| 6251 | 1569823_at | 5.95E−04 | LOC440346 |
| 5328 | 215678_at | 2.52E−05 | LOC440792 |
| 719 | 227922_x_at | 1.00E−05 | LOC441124 |
| 1034 | 215297_at | 1.00E−05 | LOC441204 |
| 5736 | 232918_at | 4.04E−04 | LOC541471 |
| 2824 | 215625_at | 1.55E−05 | LOC644450 |
| 10096 | 235884_at | 0.002 | LOC644794 |
| 6267 | 1555400_at | 7.51E−07 | LOC645261 |
| 4292 | 1568933_at | 2.48E−06 | LOC646627 |
| 796 | 215467_x_at | 1.87E−04 | LOC647070 |
| 614 | 1561492_at | 3.00E−06 | LOC647107 |
| 3588 | 240371_at | 0.002 | LOC647107 |
| 796 | 1558028_x_at | 2.74E−04 | LOC647979 |
| 9628 | 1562428_at | 9.20E−04 | LOC654780 |
| 9573 | 1564559_at | 5.12E−04 | LOC728073 |
| 2145 | 234133_s_at | 5.40E−04 | LOC728543 |
| 1599 | 1568764_x_at | 0.002 | LOC728613 /// PDCD6 |
| 1599 | 1568762_at | 0.002 | LOC728613 /// PDCD6 |
| 5170 | 1563978_at | 1.41E−04 | LOC728690 |
| 5170 | 213388_at | 3.09E−04 | LOC728802 /// PDE4DIP |
| 1145 | 1558623_at | 1.00E−09 | LOC729121 |
| 483 | 220823_at | 0.00115 | LOC729164 |
| 6251 | 1563637_at | 2.69E−06 | LOC729652 |
| 1101 | 241607_at | 0.002 | LOC730102 |
| 2135 | 1561571_at | 3.00E−06 | LOC730139 |
| 959 | 1556117_at | 5.50E−05 | LOC79015 |
| 559 | 233321_x_at | 1.25E−04 | LOC90834 |
| 5736 | 234341_x_at | 6.63E−04 | LOC91548 |
| 5873 | 233132_at | 1.02E−04 | LOC96610 |
| 5328 | 215322_at | 5.28E−05 | LONRF1 |
| 5328 | 202793_at | 1.04E−04 | LPCAT3 |
| 6131 | 242778_at | 3.50E−04 | LPXN |
| 1594 | 230863_at | 2.00E−04 | LRP2 |
| 5328 | 231781_s_at | 9.34E−05 | LRRC2 |
| 6740 | 215063_x_at | 0.001467 | LRRC40 |
| 5732 | 211452_x_at | 5.12E−04 | LRRFIP1 |
| 5328 | 215375_x_at | 8.92E−05 | LRRFIP1 |
| 8366 | 232705_at | 4.00E−05 | LRRFIP2 |
| 3465 | 223817_at | 7.94E−06 | LRRIQ1 |
| 2824 | 216167_at | 3.30E−04 | LRRN2 |
| 7009 | 221536_s_at | 3.00E−06 | LSG1 |
| 174 | 236363_at | 6.00E−04 | LSM3 |
| 7567 | 202903_at | 8.64E−04 | LSM5 |
| 2497 | 243852_at | 0.001025 | LUC7L2 |
| 7313 | 227797_x_at | 0.002 | LYRM2 |
| 9338 | 231840_x_at | 1.35E−04 | LYRM7 |
| 3592 | 232168_x_at | 5.78E−04 | MACF1 |
| 5328 | 1553407_at | 2.06E−04 | MACF1 |
| 5170 | 1559731_x_at | 3.43E−04 | MACROD1 |
| 123 | 205193_at | 7.14E−04 | MAFF |
| 4793 | 1559352_a_at | 5.03E−05 | MAFG-AS1 |
| 5930 | 224650_at | 0.002 | MAL2 |
| 8965 | 217282_at | 0.001 | MAN1A2 |
| 2824 | 220945_x_at | 1.37E−04 | MANSC1 |
| 9338 | 221697_at | 7.36E−05 | MAP1LC3C |
| 9338 | 216206_x_at | 3.53E−05 | MAP2K7 |
| 492 | 206296_x_at | 7.52E−04 | MAP4K1 |
| 10081 | 210059_s_at | 0.003 | MAPK13 |
| 10081 | 210058_at | 0.003 | MAPK13 |
| 3465 | 1553987_at | 3.25E−04 | MAPKAPK5-AS1 |
| 2108 | 232537_x_at | 1.50E−05 | MARK3 |
| 6673 | 235955_at | 1.26E−04 | MARVELD2 |
| 5328 | 206091_at | 2.28E−05 | MATN3 |
| 560 | 242260_at | 0.00105 | MATR3 |
| 6249 | 228012_at | 0.001618 | MATR3 /// SNHG4 |
| 345 | 242077_x_at | 3.03E−04 | MB21D1 |
| 10016 | 241813_at | 5.00E−05 | MBD1 |
| 6251 | 207115_x_at | 3.01E−04 | MBTD1 |
| 7331 | 211042_x_at | 0.003 | MCAM |
| 5328 | 210869_s_at | 1.30E−04 | MCAM |
| 9345 | 1560033_at | 8.58E−05 | MCCC2 |
| 5328 | 215112_x_at | 1.68E−04 | MCF2L2 |
| 5016 | 1559309_at | 0.001703 | MCFD2 |
| 5732 | 215582_x_at | 3.39E−04 | MCM3AP |
| 6249 | 1560542_at | 8.10E−04 | MCM3AP-AS1 |
| 9378 | 222036_s_at | 0.002 | MCM4 |
| 2340 | 229021_at | 9.49E−04 | MCTP2 |
| 6713 | 229711_s_at | 3.00E−06 | MDM2 |
| 7992 | 205386_s_at | 9.91E−04 | MDM2 |
| 1047 | 244616_x_at | 8.50E−04 | MDM2 |
| 5316 | 1562731_s_at | 2.01E−09 | MDS2 |
| 8147 | 219730_at | 2.76E−05 | MED18 |
| 3402 | 221650_s_at | 0.001714 | MED18 |
| 6846 | 225708_at | 1.00E−04 | MED29 |
| 2824 | 208262_x_at | 3.05E−04 | MEFV |
| 9352 | 1569402_at | 5.50E−05 | MEIS3 |
| 1047 | 224672_x_at | 8.13E−04 | MESDC2 |
| 3465 | 238931_at | 1.60E−05 | METTL16 |
| 1043 | 1554582_a_at | 1.59E−04 | METTL20 |
| 416 | 233742_at | 1.01E−04 | METTL22 |
| 4267 | 242111_at | 3.00E−06 | METTL3 |
| 9365 | 1557991_at | 0.001073 | METTL6 |
| 2824 | 1553688_at | 1.37E−04 | METTL6 |
| 1717 | 211424_x_at | 1.43E−04 | METTL7A |
| 1717 | 209703_x_at | 1.43E−04 | METTL7A |
| 5873 | 221192_x_at | 1.74E−04 | MFSD11 |
| 7300 | 224134_at | 8.08E−06 | MGC10814 |
| 7214 | 1560019_at | 2.00E−07 | MGC11082 |
| 3465 | 211386_at | 3.07E−04 | MGC12488 |
| 5170 | 211775_x_at | 4.01E−04 | MGC13053 |
| 9397 | 1553881_at | 5.42E−05 | MGC16142 |
| 2824 | 1558167_a_at | 1.51E−04 | MGC16275 |
| 2824 | 208136_s_at | 1.33E−04 | MGC3771 |
| 4089 | 219332_at | 1.00E−08 | MICALL2 |
| 416 | 206247_at | 9.68E−05 | MICB |
| 492 | 243760_at | 0.001348 | MIPEPP3 |
| 5043 | 1569348_at | 2.92E−04 | MIR4500HG /// TPTEP1 |
| 2824 | 212594_at | 1.92E−05 | MIR4680 /// PDCD4 |
| 1043 | 232169_x_at | 2.20E−04 | MIR4691 /// NDUFS8 |
| 9338 | 206500_s_at | 1.03E−04 | MIS18BP1 |
| 4409 | 226066_at | 0.002 | MITF |
| 6431 | 234167_at | 4.00E−05 | MKI67IP |
| 5736 | 1560145_at | 7.24E−05 | MKLN1 |
| 5170 | 206426_at | 3.76E−04 | MLANA |
| 796 | 205989_s_at | 1.25E−05 | MOG |
| 5328 | 214650_x_at | 7.80E−05 | MOG |
| 5043 | 1559167_x_at | 0.001005 | MPV17L |
| 5937 | 210224_at | 6.55E−04 | MR1 |
| 4331 | 210528_at | 9.80E−04 | MR1 |
| 115 | 223770_x_at | 2.61E−05 | MRI1 |
| 3465 | 204387_x_at | 7.45E−05 | MRP63 |
| 6092 | 213897_s_at | 1.00E−21 | MRPL23 |
| 2045 | 1562392_at | 0.001331 | MRPL23 |
| 9345 | 1554468_s_at | 3.79E−05 | MRPL38 |
| 5328 | 218202_x_at | 8.90E−05 | MRPL44 |
| 921 | 215919_s_at | 5.00E−09 | MRPS11 |
| 9477 | 224015_s_at | 0.001875 | MRPS25 |
| 4793 | 235783_at | 1.86E−05 | MRTO4 |
| 2824 | 205887_x_at | 1.85E−04 | MSH3 |
| 7421 | 227277_at | 1.35E−04 | MTDH |
| 4579 | 234976_x_at | 1.59E−04 | MTHFD2 |
| 5170 | 1562644_at | 4.93E−05 | MTHFD2L |
| 5969 | 228789_at | 8.00E−07 | MTMR6 |
| 4331 | 233873_x_at | 4.00E−04 | MTPAP |
| 559 | 215560_x_at | 2.91E−04 | MTRF1L |
| 9338 | 217295_at | 3.22E−06 | MUC8 |
| 2256 | 36907_at | 5.00E−04 | MVK |
| 2256 | 204056_s_at | 5.00E−04 | MVK |
| 1562 | 216188_at | 0.001114 | MYCNOS |
| 5170 | 201497_x_at | 2.48E−04 | MYH11 |
| 6717 | 224823_at | 0.002 | MYLK |
| 279 | 1568926_x_at | 0.003 | MYLK3 |
| 279 | 1568925_at | 0.003 | MYLK3 |
| 9403 | 214656_x_at | 1.00E−07 | MYO1C |
| 66 | 244654_at | 6.93E−04 | MYO1G |
| 4236 | 227799_at | 1.00E−06 | MYO1G |
| 6249 | 242512_at | 0.001696 | MYO9A |
| 5328 | 220720_x_at | 2.83E−04 | MZT2B |
| 2168 | 231039_at | 0.002 | NAA30 |
| 7864 | 218661_at | 3.00E−06 | NAA60 |
| 7864 | 45526_g_at | 3.00E−06 | NAA60 |
| 8835 | 215178_x_at | 1.00E−04 | NAAA |
| 8104 | 216017_s_at | 0.001 | NAB2 |
| 5736 | 211445_x_at | 1.44E−04 | NACAP1 |
| 790 | 1554702_at | 7.50E−06 | NALCN |
| 1407 | 241923_x_at | 2.88E−04 | NANS |
| 9352 | 238722_x_at | 1.20E−04 | NAPEPLD |
| 8015 | 1564658_at | 3.30E−04 | NAT16 |
| 2045 | 233870_at | 6.00E−05 | NAV1 |
| 7645 | 216466_at | 2.36E−04 | NAV3 |
| 3210 | 1553910_at | 1.00E−05 | NBPF4 |
| 115 | 207631_at | 2.91E−05 | NBR2 |
| 4793 | 216114_at | 6.50E−05 | NCKIPSD |
| 2824 | 215857_at | 4.86E−05 | NCLN |
| 2168 | 207760_s_at | 6.00E−04 | NCOR2 |
| 8892 | 207860_at | 5.08E−04 | NCR1 |
| 1943 | 230219_at | 5.00E−04 | NDE1 |
| 5328 | 215850_s_at | 3.00E−05 | NDUFA5 |
| 378 | 243630_at | 3.00E−04 | NDUFB1 |
| 3592 | 222521_x_at | 5.90E−04 | NDUFC2 /// NDUFC2-KCTD14 |
| 5736 | 239268_at | 4.31E−04 | NDUFS1 |
| 8303 | 229647_at | 5.00E−04 | NDUFS1 |
| 6846 | 1564093_at | 0.001687 | NEK1 |
| 5638 | 1554659_at | 5.00E−04 | NEK4 |
| 5170 | 1557172_x_at | 4.99E−04 | NEK8 |
| 2110 | 216115_at | 0.001163 | NF1 |
| 8453 | 217526_at | 1.00E−06 | NFATC2IP |
| 5736 | 210556_at | 1.81E−04 | NFATC3 |
| 123 | 207492_at | 3.13E−04 | NGLY1 |
| 2824 | 1568974_at | 5.55E−05 | NHEG1 |
| 5328 | 219418_at | 3.89E−04 | NHEJ1 /// SLC23A3 |
| 7020 | 227040_at | 6.00E−19 | NHLRC3 |
| 6811 | 232158_x_at | 1.77E−04 | NIPAL1 |
| 2137 | 241395_at | 0.00102 | NIT1 |
| 3620 | 1557737_s_at | 1.35E−05 | NKTR |
| 796 | 234762_x_at | 2.95E−04 | NLN |
| 3465 | 225942_at | 4.50E−04 | NLN |
| 4416 | 221690_s_at | 0.002 | NLRP2 |
| 5115 | 216015_s_at | 0.002 | NLRP3 |
| 5115 | 216016_at | 0.002 | NLRP3 |
| 214 | 215069_at | 4.00E−05 | NMT2 |
| 287 | 238530_at | 0.00105 | NNT |
| 483 | 1559139_at | 8.18E−04 | NOC2L |
| 5170 | 1558738_at | 2.92E−04 | NOL3 |
| 9628 | 229220_x_at | 2.43E−04 | NOM1 |
| 7984 | 242922_at | 4.67E−05 | NOMO3 |
| 1129 | 214123_s_at | 4.03E−04 | NOP14-AS1 |
| 4043 | 1555390_at | 0.004 | NOP9 |
| 4579 | 217584_at | 1.69E−04 | NPC1 |
| 416 | 206285_at | 3.30E−04 | NPHP1 |
| 1043 | 1553389_at | 9.90E−05 | NPHP3 |
| 2824 | 235432_at | 2.25E−04 | NPHP3 |
| 7908 | 204538_x_at | 6.67E−13 | NPIP |
| 7908 | 221992_at | 1.00E−07 | NPIPL2 |
| 5736 | 236860_at | 1.07E−04 | NPY6R |
| 3465 | 1568913_at | 1.77E−04 | NSUN3 |
| 5170 | 222886_at | 4.53E−04 | NSUN3 |
| 5170 | 209155_s_at | 1.39E−04 | NT5C2 |
| 559 | 234737_at | 6.28E−04 | NT5DC3 |
| 8366 | 1569030_s_at | 2.40E−05 | NUB1 |
| 5328 | 234332_at | 7.96E−05 | NUB1 |
| 5328 | 219347_at | 1.81E−04 | NUDT15 |
| 7129 | 232251_at | 0.00105 | NUDT16P1 |
| 2110 | 1554663_a_at | 0.001398 | NUMA1 |
| 5736 | 214675_at | 1.02E−04 | NUP188 |
| 3697 | 219007_at | 5.62E−04 | NUP43 |
| 5891 | 218039_at | 7.00E−07 | NUSAP1 |
| 9051 | 1553755_at | 4.85E−04 | NXNL1 |
| 5222 | 221684_s_at | 5.00E−04 | NYX |
| 7461 | 232644_x_at | 0.00155 | OCIAD1 |
| 5328 | 239748_x_at | 6.49E−05 | OCIAD1 |
| 3468 | 205760_s_at | 0.002 | OGG1 |
| 2110 | 219975_x_at | 7.37E−04 | OLAH |
| 3402 | 229624_at | 4.60E−04 | OPA3 |
| 6358 | 206323_x_at | 3.00E−05 | OPHN1 |
| 2584 | 222290_at | 4.00E−05 | OR2A20P /// OR2A9P |
| 4425 | 1566956_at | 1.60E−06 | OR7E104P |
| 7567 | 228676_at | 4.15E−04 | ORAOV1 |
| 5170 | 205085_at | 2.87E−04 | ORC1 |
| 5328 | 219105_x_at | 1.37E−04 | ORC6 |
| 559 | 1563051_at | 3.20E−05 | OSBP |
| 9397 | 216755_at | 2.05E−04 | OSBPL10 |
| 8431 | 1554503_a_at | 4.46E−04 | OSCAR |
| 1964 | 230032_at | 0.001001 | OSGEPL1 |
| 1043 | 206048_at | 0.001158 | OVOL2 |
| 4012 | 229686_at | 7.00E−05 | P2RY8 |
| 5043 | 1560715_at | 7.94E−05 | P4HA3 |
| 4621 | 1560770_at | 7.12E−04 | PABPC1 |
| 7984 | 235554_x_at | 2.61E−04 | PACRGL |
| 5043 | 211412_at | 2.22E−04 | PADI4 |
| 2824 | 205233_s_at | 6.41E−05 | PAFAH2 |
| 8270 | 201013_s_at | 4.50E−04 | PAICS |
| 1562 | 214664_at | 6.57E−04 | PAICS |
| 1129 | 215374_at | 4.16E−05 | PAPOLA |
| 8355 | 227348_at | 1.00E−04 | PARS2 |
| 5889 | 1563458_at | 6.50E−04 | PARVA |
| 2017 | 235279_at | 3.00E−06 | PARVA |
| 8278 | 244342_at | 2.00E−07 | PATL1 |
| 2168 | 213917_at | 5.00E−05 | PAX8 |
| 6249 | 217049_x_at | 3.71E−04 | PCDH11X /// PCDH11Y |
| 5328 | 223927_at | 1.53E−04 | PCDHB9 |
| 8147 | 210368_at | 6.18E−04 | PCDHGA8 |
| 3465 | 237577_at | 0.002 | PCNP |
| 2824 | 217624_at | 2.56E−04 | PDAP1 |
| 5937 | 231809_x_at | 4.78E−04 | PDCD7 |
| 3676 | 204449_at | 3.77E−04 | PDCL |
| 7992 | 214826_at | 0.001679 | PDE12 |
| 1043 | 206792_x_at | 3.64E−04 | PDE4C |
| 1138 | 204491_at | 0.002 | PDE4D |
| 2824 | 210304_at | 3.93E−04 | PDE6B |
| 9338 | 1552343_s_at | 2.45E−05 | PDE7A |
| 9345 | 1562321_at | 1.05E−05 | PDK4 |
| 1562 | 208233_at | 5.10E−04 | PDPN |
| 5328 | 221898_at | 2.00E−04 | PDPN |
| 1735 | 225075_at | 6.00E−04 | PDRG1 |
| 2824 | 1560014_s_at | 4.62E−05 | PDXDC1 |
| 2824 | 1560013_at | 4.81E−04 | PDXDC1 |
| 5328 | 1566670_at | 6.04E−05 | PDXK |
| 5216 | 223619_x_at | 2.08E−04 | PECR |
| 4907 | 213791_at | 4.77E−05 | PENK |
| 3867 | 1557476_at | 3.00E−06 | PFN4 |
| 4579 | 215179_x_at | 4.52E−04 | PGF |
| 7472 | 221788_at | 0.0025 | PGM3 |
| 3676 | 231812_x_at | 4.36E−04 | PHAX |
| 4579 | 235767_x_at | 5.43E−04 | PHAX |
| 8819 | 209422_at | 0.002002 | PHF20 |
| 8156 | 1558965_at | 5.43E−04 | PHF21A |
| 8303 | 1559705_s_at | 4.49E−05 | PHKA2 |
| 5736 | 202739_s_at | 1.64E−04 | PHKB |
| 5736 | 210919_at | 3.43E−04 | PHLPP1 |
| 5860 | 205325_at | 0.003 | PHYHIP |
| 3465 | 1566111_at | 2.45E−04 | PIEZO1 |
| 1457 | 1555394_at | 0.004 | PIGK |
| 6249 | 1568986_x_at | 6.43E−04 | PIGT |
| 4268 | 235792_x_at | 8.59E−05 | PIK3C2A |
| 4331 | 206370_at | 4.64E−04 | PIK3CG |
| 7472 | 216436_at | 0.0011 | PIK3R4 |
| 7331 | 219788_at | 0.001799 | PILRA |
| 5170 | 221605_s_at | 3.27E−04 | PIPOX |
| 5732 | 1563465_at | 2.70E−04 | PKD1L1 |
| 5170 | 224226_at | 5.89E−04 | PKD2L2 |
| 187 | 204196_x_at | 5.20E−06 | PKNOX1 |
| 378 | 214750_at | 7.85E−04 | PLAC4 |
| 5328 | 234971_x_at | 8.50E−04 | PLCD3 |
| 5328 | 1552476_s_at | 4.18E−04 | PLCD3 |
| 9338 | 1566740_at | 3.74E−04 | PLCE1 |
| 3465 | 224892_at | 6.33E−04 | PLDN |
| 492 | 217677_at | 7.38E−04 | PLEKHA2 |
| 6251 | 1558407_at | 2.35E−05 | PLEKHG2 |
| 559 | 236255_at | 9.13E−05 | PLEKHG4B |
| 8015 | 218290_at | 0.0012 | PLEKHJ1 |
| 5170 | 219857_at | 4.48E−04 | PLEKHS1 |
| 4268 | 215895_x_at | 2.08E−05 | PLIN2 |
| 5328 | 202122_s_at | 8.44E−05 | PLIN3 |
| 345 | 241368_at | 4.80E−04 | PLIN5 |
| 5328 | 215462_at | 3.39E−05 | PLK3 |
| 4973 | 235127_at | 3.00E−06 | PMP2 |
| 8908 | 214756_x_at | 3.60E−04 | PMS2P1 |
| 8908 | 217485_x_at | 3.60E−04 | PMS2P1 |
| 9352 | 210722_at | 9.88E−05 | PNLIPRP1 |
| 796 | 211697_x_at | 2.18E−04 | PNO1 |
| 3465 | 201695_s_at | 8.57E−04 | PNP |
| 2824 | 1553364_at | 2.08E−04 | PNPLA1 |
| 2824 | 220411_x_at | 3.01E−04 | PODNL1 |
| 6774 | 222564_at | 0.003 | POGK |
| 8366 | 215281_x_at | 3.59E−04 | POGZ |
| 8132 | 244575_at | 0.001848 | POLA2 |
| 1562 | 208828_at | 8.20E−04 | POLE3 |
| 1885 | 233852_at | 8.58E−04 | POLH |
| 5170 | 219380_x_at | 3.08E−04 | POLH |
| 796 | 1555317_at | 9.39E−06 | POLK |
| 9628 | 235280_at | 7.12E−05 | POLR1A |
| 123 | 220113_x_at | 5.34E−04 | POLR1B |
| 3465 | 233341_s_at | 5.00E−04 | POLR1B |
| 9628 | 218997_at | 2.62E−04 | POLR1E |
| 8908 | 216242_x_at | 3.60E−04 | POLR2J2 /// POLR2J3 /// POLR2J4 /// UPK3BL |
| 416 | 205096_at | 1.05E−04 | POM121 |
| 4808 | 207109_at | 2.84E−04 | POU2F3 |
| 9365 | 1555322_at | 7.45E−04 | PP2672 |
| 559 | 244689_at | 2.02E−04 | PPARA |
| 4331 | 210771_at | 4.23E−04 | PPARA |
| 5328 | 210636_at | 3.79E−04 | PPARD |
| 2824 | 208411_x_at | 1.38E−04 | PPEF2 |
| 5170 | 203735_x_at | 1.79E−04 | PPFIBP1 |
| 560 | 225073_at | 3.00E−04 | PPHLN1 |
| 5328 | 204185_x_at | 1.58E−04 | PPID |
| 9797 | 236754_at | 0.002 | PPP1R2 |
| 2824 | 1563690_at | 3.23E−05 | PPP4R1 |
| 9345 | 1570210_x_at | 4.76E−04 | PPP6R2 |
| 5328 | 238110_at | 2.22E−05 | PPP6R2 |
| 8430 | 232312_at | 5.00E−04 | PPP6R3 |
| 4331 | 236999_at | 3.50E−04 | PPWD1 |
| 8319 | 1555783_x_at | 4.50E−05 | PQLC2 |
| 8319 | 1555781_at | 4.50E−05 | PQLC2 |
| 5328 | 220714_at | 8.37E−05 | PRDM14 |
| 492 | 215067_x_at | 9.60E−04 | PRDX2 |
| 521 | 200845_s_at | 1.00E−08 | PRDX6 |
| 7249 | 220732_at | 5.00E−04 | PREX2 |
| 9338 | 232811_x_at | 7.36E−05 | PRICKLE1 |
| 4482 | 1561676_at | 5.00E−05 | PRICKLE2-AS3 |
| 4104 | 217349_s_at | 0.003 | PRICKLE3 |
| 9051 | 205628_at | 8.76E−04 | PRIM2 |
| 9397 | 238489_at | 2.49E−04 | PRKAA2 |
| 559 | 204842_x_at | 2.00E−04 | PRKAR2A |
| 1921 | 214080_x_at | 0.005 | PRKCSH |
| 2824 | 1569748_at | 1.39E−04 | PRKCZ |
| 5115 | 231758_at | 5.00E−04 | PRM3 |
| 6843 | 213320_at | 6.00E−04 | PRMT3 |
| 5328 | 222106_at | 1.28E−04 | PRND |
| 1101 | 222965_at | 0.002 | PRO2214 |
| 559 | 230270_at | 3.94E−04 | PRPF38B |
| 347 | 232215_x_at | 2.99E−04 | PRR11 |
| 5328 | 219392_x_at | 1.40E−04 | PRR11 |
| 5736 | 1553761_at | 5.78E−04 | PRR14L |
| 5170 | 1560448_at | 1.94E−04 | PRR7-AS1 |
| 2824 | 207291_at | 1.82E−05 | PRRG4 |
| 5736 | 1556308_at | 2.14E−04 | PRRT3 |
| 6936 | 202458_at | 6.00E−04 | PRSS23 |
| 8354 | 229441_at | 0.003 | PRSS23 |
| 5328 | 222255_at | 5.84E−05 | PRX |
| 796 | 1559206_at | 9.87E−05 | PSEN1 |
| 1340 | 232648_at | 0.00155 | PSMA3 |
| 7700 | 238425_at | 0.001133 | PSMB2 |
| 1047 | 201404_x_at | 1.28E−04 | PSMB2 |
| 5170 | 205194_at | 1.87E−04 | PSPH |
| 2602 | 233254_x_at | 0.001 | PTEN |
| 2824 | 211265_at | 3.55E−04 | PTGER3 |
| 4267 | 1559529_at | 2.38E−04 | PTK2 |
| 2824 | 206482_at | 8.74E−05 | PTK6 |
| 2824 | 1555324_at | 1.47E−04 | PTK7 |
| 5170 | 1554376_s_at | 3.20E−04 | PTPLA |
| 10019 | 243792_x_at | 0.00164 | PTPN13 |
| 3020 | 242321_at | 5.45E−05 | PTPN14 |
| 538 | 244533_at | 5.44E−04 | PTPN14 |
| 796 | 203555_at | 8.64E−04 | PTPN18 |
| 4808 | 232161_x_at | 6.62E−05 | PTPN4 |
| 3508 | 230250_at | 0.005 | PTPRB |
| 559 | 215066_at | 8.14E−05 | PTPRF |
| 2824 | 218984_at | 0.001688 | PUS7 |
| 4331 | 223540_at | 8.61E−05 | PVRL4 |
| 7214 | 1565604_at | 2.00E−07 | PWP2 |
| 559 | 241933_at | 1.24E−04 | QRSL1 |
| 8453 | 233089_at | 1.00E−06 | QRSL1 |
| 5328 | 1560696_x_at | 2.26E−05 | QTRTD1 |
| 5328 | 1560695_at | 2.26E−05 | QTRTD1 |
| 9365 | 1556123_a_at | 0.002 | RAB11B-AS1 |
| 2824 | 231830_x_at | 3.19E−04 | RAB11FIP1 |
| 5170 | 225746_at | 0.001239 | RAB11FIP4 |
| 1043 | 1554800_at | 8.09E−04 | RAB39A |
| 5170 | 1565800_x_at | 1.09E−04 | RAB3IP |
| 5170 | 1565799_at | 1.09E−04 | RAB3IP |
| 492 | 1569396_at | 3.23E−04 | RAB40C |
| 932 | 1558021_at | 6.20E−06 | RABEPK |
| 5170 | 204477_at | 3.16E−04 | RABIF |
| 1407 | 226090_x_at | 6.99E−04 | RABL3 |
| 5736 | 204461_x_at | 2.32E−04 | RAD1 |
| 5117 | 205023_at | 2.08E−05 | RAD51 |
| 5736 | 215162_at | 1.66E−04 | RALGAPA1 |
| 5652 | 234923_at | 0.003 | RALGAPA1 |
| 5328 | 1555452_at | 5.38E−05 | RALGPS1 |
| 2824 | 212706_at | 4.25E−04 | RASA4 /// RASA4B |
| 5328 | 217201_at | 5.75E−05 | RASAL2 |
| 1265 | 220680_at | 0.002 | RAVER2 |
| 2824 | 1552311_a_at | 1.83E−04 | RAX2 |
| 5328 | 205169_at | 2.11E−05 | RBBP5 |
| 6249 | 205296_at | 7.45E−04 | RBL1 |
| 7481 | 200997_at | 0.005 | RBM14-RBM4 /// RBM4 |
| 4579 | 208319_s_at | 7.76E−05 | RBM3 |
| 559 | 227979_at | 1.02E−04 | RBM4 |
| 5968 | 221596_s_at | 2.88E−04 | RBM48 |
| 5328 | 1560322_at | 1.06E−04 | RBMS3 |
| 379 | 231961_at | 2.45E−05 | RBPMS |
| 483 | 207836_s_at | 1.28E−04 | RBPMS |
| 483 | 207837_at | 1.28E−04 | RBPMS |
| 6811 | 217775_s_at | 1.43E−06 | RDH11 |
| 2548 | 216153_x_at | 3.00E−06 | RECK |
| 3509 | 1558116_x_at | 5.00E−05 | RECK |
| 3509 | 1558115_at | 5.00E−05 | RECK |
| 9345 | 203225_s_at | 2.75E−05 | RFK |
| 123 | 1552809_at | 9.05E−04 | RFX4 |
| 9628 | 209638_x_at | 2.93E−04 | RGS12 |
| 6358 | 233164_x_at | 8.12E−04 | RHBDD1 |
| 5328 | 210429_at | 1.02E−05 | RHD |
| 6249 | 240111_at | 1.81E−04 | RHOBTB3 |
| 10019 | 1559582_at | 2.63E−04 | RHOQ |
| 2824 | 227196_at | 1.60E−04 | RHPN2 |
| 6249 | 233811_at | 0.001386 | RIN2 |
| 8303 | 215588_x_at | 7.10E−04 | RIOK3 |
| 8269 | 209941_at | 3.00E−05 | RIPK1 |
| 6084 | 220329_s_at | 1.70E−05 | RMND1 |
| 483 | 207735_at | 7.40E−06 | RNF125 |
| 6548 | 201824_at | 5.00E−04 | RNF14 |
| 8430 | 1556797_at | 0.002 | RNF144A-AS1 |
| 9628 | 211387_x_at | 5.87E−04 | RNGTT |
| 5016 | 1562682_at | 0.002 | RORA |
| 4220 | 1565759_at | 0.0035 | RPL13 |
| 796 | 1563581_at | 4.24E−05 | RPL34-AS1 |
| 9338 | 215208_x_at | 2.44E−04 | RPL35A |
| 492 | 224738_x_at | 5.15E−04 | RPL7L1 |
| 8306 | 217670_at | 0.00115 | RPLP2 |
| 5736 | 1563014_at | 4.27E−04 | RPS15 |
| 9628 | 1566079_at | 5.11E−04 | RPS16P5 |
| 7645 | 211487_x_at | 0.002 | RPS17 /// RPS17L |
| 4064 | 242451_x_at | 3.02E−04 | RPS19 |
| 5328 | 216247_at | 1.38E−04 | RPS20 /// SNORD54 |
| 5736 | 207489_at | 2.90E−04 | RPS2P45 |
| 1129 | 1557981_at | 1.50E−05 | RPS9 |
| 7869 | 1555916_at | 8.63E−04 | RPUSD3 |
| 2824 | 227888_at | 2.20E−04 | RRBP1 |
| 2824 | 222733_x_at | 1.57E−04 | RRP1 |
| 4793 | 1559127_x_at | 1.77E−04 | RRP12 |
| 4793 | 1559126_at | 3.67E−05 | RRP12 |
| 1043 | 242625_at | 2.86E−04 | RSAD2 |
| 5170 | 212019_at | 1.32E−04 | RSL1D1 |
| 4331 | 230093_at | 1.78E−04 | RSPH1 |
| 8763 | 1555272_at | 3.34E−04 | RSPH10B /// RSPH10B2 |
| 1562 | 215618_at | 9.26E−04 | RSU1 |
| 5170 | 230490_x_at | 6.97E−04 | RSU1 |
| 2824 | 238087_at | 1.45E−04 | RTCA |
| 7421 | 229334_at | 6.57E−04 | RUFY3 |
| 2497 | 1559882_at | 3.34E−05 | SAMHD1 |
| 5328 | 235529_x_at | 2.23E−04 | SAMHD1 |
| 5328 | 234987_at | 1.85E−05 | SAMHD1 |
| 9628 | 235964_x_at | 2.13E−04 | SAMHD1 |
| 3465 | 207889_at | 3.39E−04 | SARDH |
| 5526 | 234491_s_at | 1.67E−05 | SAV1 |
| 1047 | 216161_at | 2.20E−04 | SBNO1 |
| 4268 | 232597_x_at | 2.15E−05 | SCAF11 |
| 4579 | 206021_at | 3.53E−05 | SCAND2 |
| 1129 | 232421_at | 4.03E−04 | SCARB1 |
| 8198 | 215754_at | 0.001 | SCARB2 |
| 2110 | 206995_x_at | 9.07E−04 | SCARF1 |
| 6628 | 220232_at | 3.00E−13 | SCD5 |
| 5328 | 222272_x_at | 5.76E−04 | SCIN |
| 2824 | 206147_x_at | 7.40E−05 | SCML2 |
| 6249 | 220791_x_at | 6.08E−04 | SCN11A |
| 2824 | 224029_x_at | 2.53E−04 | SCN11A |
| 796 | 210364_at | 7.86E−06 | SCN2B |
| 1594 | 218681_s_at | 0.00185 | SDF2L1 |
| 63 | 1558919_a_at | 2.27E−04 | SEC231P |
| 4786 | 202375_at | 7.00E−04 | SEC24D |
| 9397 | 205405_at | 2.37E−05 | SEMA5A |
| 1047 | 208671_at | 4.74E−04 | SERINC1 |
| 9345 | 1558727_at | 4.75E−04 | SERP2 |
| 1129 | 209723_at | 6.44E−04 | SERPINB9 |
| 4579 | 1564972_x_at | 5.63E−05 | SETDB2 |
| 4579 | 1564970_at | 5.63E−05 | SETDB2 |
| 7783 | 232487_at | 0.002 | SFT2D1 |
| 5736 | 213936_x_at | 5.14E−04 | SFTPB |
| 6515 | 214354_x_at | 5.18E−05 | SFTPB |
| 7991 | 218392_x_at | 1.21E−04 | SFXN1 |
| 6811 | 220974_x_at | 2.84E−04 | SFXN3 |
| 5328 | 231938_at | 2.52E−04 | SGOL1 |
| 9020 | 217538_at | 3.35E−05 | SGSM2 |
| 5328 | 222169_x_at | 7.38E−05 | SH2D3A |
| 5328 | 217253_at | 5.54E−05 | SH3BP2 |
| 3465 | 205636_at | 1.04E−04 | SH3GL3 |
| 4579 | 1564377_at | 2.56E−04 | SHANK2 |
| 123 | 224139_at | 0.001335 | SHANK2-AS3 |
| 559 | 219493_at | 3.32E−04 | SHCBP1 |
| 4081 | 202980_s_at | 1.00E−04 | SIAH1 |
| 2524 | 1552910_at | 2.63E−04 | SIGLEC11 |
| 2824 | 215856_at | 1.36E−04 | SIGLEC15 |
| 3548 | 208253_at | 3.00E−06 | SIGLEC8 |
| 5328 | 208157_at | 2.83E−05 | SIM2 |
| 10019 | 1558331_at | 7.35E−04 | SIRT2 |
| 2824 | 1569938_at | 1.80E−05 | SIRT5 |
| 5115 | 206634_at | 0.002 | SIX3 |
| 5732 | 204362_at | 0.0015 | SKAP2 |
| 416 | 215889_at | 1.05E−04 | SKIL |
| 1340 | 1562142_at | 3.14E−04 | SKIV2L2 |
| 4579 | 1552497_a_at | 5.58E−05 | SLAMF6 |
| 4579 | 217473_x_at | 6.69E−04 | SLC11A1 |
| 4579 | 210422_x_at | 6.69E−04 | SLC11A1 |
| 9352 | 210047_at | 8.00E−05 | SLC11A2 |
| 2136 | 215274_at | 0.0016 | SLC12A3 |
| 9022 | 211349_at | 0.003 | SLC15A1 |
| 7567 | 223441_at | 7.00E−05 | SLC17A5 |
| 716 | 242578_x_at | 4.00E−04 | SLC22A3 |
| 3465 | 1553265_at | 2.00E−04 | SLC23A3 |
| 9397 | 1568922_at | 1.99E−05 | SLC24A4 |
| 2824 | 211855_s_at | 1.58E−05 | SLC25A14 |
| 9628 | 1569793_at | 2.21E−04 | SLC25A18 |
| 9628 | 220182_at | 1.80E−04 | SLC25A23 |
| 5170 | 206529_x_at | 2.72E−04 | SLC26A4 |
| 5316 | 207249_s_at | 1.14E−04 | SLC28A2 |
| 4405 | 1560062_at | 9.00E−05 | SLC29A2 |
| 4268 | 232167_at | 1.07E−05 | SLC2A11 |
| 9628 | 1558832_at | 4.54E−04 | SLC2A1-AS1 |
| 3212 | 202497_x_at | 3.00E−04 | SLC2A3 |
| 5328 | 220091_at | 6.37E−05 | SLC2A6 |
| 5652 | 238845_at | 6.00E−04 | SLC30A4 |
| 4808 | 220181_x_at | 1.82E−04 | SLC30A5 |
| 4268 | 243643_x_at | 3.00E−06 | SLC30A6 |
| 1043 | 203971_at | 2.49E−04 | SLC31A1 |
| 4331 | 236217_at | 0.002 | SLC31A1 |
| 483 | 204124_at | 1.03E−04 | SLC34A2 |
| 1043 | 220796_x_at | 5.55E−04 | SLC35E1 |
| 18 | 230694_at | 4.00E−08 | SLC35E2B |
| 8394 | 230448_at | 1.00E−06 | SLC38A10 |
| 5170 | 1555433_at | 2.03E−04 | SLC39A14 |
| 7421 | 233123_at | 3.39E−04 | SLC40A1 |
| 7815 | 1552782_at | 1.85E−05 | SLC44A5 |
| 2824 | 219125_s_at | 2.29E−05 | SLC50A1 |
| 984 | 244620_at | 3.00E−06 | SLC8A1-AS1 |
| 796 | 1555142_at | 2.32E−05 | SLC9B1 |
| 9831 | 211481_at | 3.10E−05 | SLCO1A2 |
| 211 | 238430_x_at | 0.001184 | SLFN5 |
| 2136 | 216216_at | 0.002 | SLIT3 |
| 5016 | 210580_x_at | 0.001439 | SLX1A-SULT1A3 /// SLX1B-SULT1A4 /// SULT1A3 /// SULT1A4 |
| 483 | 206565_x_at | 7.43E−04 | SMA4 |
| 9338 | 1569073_x_at | 9.59E−05 | SMARCA4 |
| 6131 | 1556925_at | 0.00125 | SMC3 |
| 8303 | 215623_x_at | 2.00E−04 | SMC4 |
| 6071 | 1553020_at | 3.00E−04 | SMCR5 |
| 5969 | 233946_at | 8.00E−07 | SMU1 |
| 8165 | 222286_at | 1.00E−04 | SNAPC3 |
| 2173 | 224610_at | 7.00E−05 | SNHG1 /// SNORD22 /// SNORD25 /// SNORD26 /// SNORD27 /// |
| | | | SNORD28 /// SNORD29 /// SNORD31 |
| 6251 | 229209_at | 2.16E−04 | SNHG10 |
| 2824 | 1564911_at | 2.07E−04 | SNHG4 |
| 2824 | 232355_at | 2.07E−04 | SNORD114-3 |
| 67 | 226263_at | 0.002026 | SNRNP48 |
| 5170 | 1560741_at | 2.83E−04 | SNRPN /// SNURF |
| 7992 | 222183_x_at | 4.67E−04 | SNW1 |
| 9338 | 205482_x_at | 2.18E−04 | SNX15 |
| 6634 | 234098_at | 6.04E−04 | SOBP |
| 5043 | 215852_x_at | 3.39E−04 | SOGA1 |
| 6675 | 230782_at | 3.00E−06 | SORD |
| 5115 | 211665_s_at | 5.00E−04 | SOS2 |
| 5736 | 235526_at | 5.15E−04 | SOX6 |
| 7848 | 226913_s_at | 1.00E−05 | SOX8 |
| 9338 | 214791_at | 6.65E−05 | SP140L |
| 1752 | 216989_at | 0.003333 | SPAM1 |
| 5170 | 1570130_at | 3.26E−04 | SPATS2 |
| 3592 | 217610_at | 6.26E−04 | SPDYE2 |
| 4793 | 215383_x_at | 8.10E−05 | SPG21 |
| 5170 | 235325_at | 1.19E−04 | SPG7 |
| 6325 | 206318_at | 2.48E−04 | SPINLW1 /// SPINLW1-WFDC6 |
| 6520 | 1569723_a_at | 2.17E−04 | SPIRE2 |
| 5170 | 243594_x_at | 3.13E−04 | SPIRE2 |
| 796 | 206056_x_at | 3.45E−04 | SPN |
| 796 | 206057_x_at | 2.36E−04 | SPN |
| 796 | 216981_x_at | 2.36E−04 | SPN |
| 5328 | 1568964_x_at | 3.65E−04 | SPN |
| 5736 | 1562415_a_at | 4.07E−05 | SPOCD1 |
| 1129 | 210693_at | 1.19E−04 | SPPL2B |
| 8763 | 214925_s_at | 7.50E−05 | SPTAN1 |
| 3588 | 243361_at | 1.00E−04 | SREK1 |
| 6173 | 1554676_at | 0.001275 | SRGN |
| 1885 | 232392_at | 9.64E−04 | SRSF3 |
| 3660 | 242837_at | 0.002 | SRSF4 |
| 6973 | 204963_at | 0.004 | SSPN |
| 2089 | 210242_x_at | 2.14E−04 | ST20 |
| 2089 | 217104_at | 2.14E−04 | ST20 |
| 559 | 208322_s_at | 0.00116 | ST3GAL1 |
| 9338 | 217650_x_at | 1.34E−04 | ST3GAL2 |
| 5270 | 202440_s_at | 5.00E−04 | ST5 |
| 5170 | 232588_at | 2.80E−04 | STAG1 |
| 7501 | 203544_s_at | 0.002 | STAM |
| 9397 | 242569_at | 8.00E−06 | STAM2 |
| 194 | 206868_at | 5.00E−04 | STARD8 |
| 5736 | 1554686_at | 1.34E−04 | STAU2 |
| 9748 | 1556185_a_at | 5.00E−06 | STEAP4 |
| 1961 | 1563496_at | 3.00E−06 | STL |
| 5636 | 201060_x_at | 9.17E−05 | STOM |
| 8960 | 1553202_at | 0.001 | STOX1 |
| 10019 | 1569815_x_at | 9.04E−04 | STRN |
| 10019 | 1569813_at | 9.04E−04 | STRN |
| 5577 | 212111_at | 0.00195 | STX12 |
| 559 | 226662_at | 2.16E−04 | STX17 |
| 7992 | 1555614_at | 3.35E−04 | SUGT1P1 |
| 2045 | 215299_x_at | 0.002333 | SULT1A1 |
| 6008 | 222940_at | 2.00E−04 | SULT1E1 |
| 1043 | 206293_at | 3.84E−04 | SULT2A1 |
| 5328 | 225002_s_at | 5.16E−05 | SUMF2 |
| 5170 | 206487_at | 3.16E−04 | SUN1 |
| 3646 | 1566191_at | 1.62E−04 | SUZ12 |
| 2824 | 217704_x_at | 5.20E−05 | SUZ12P |
| 790 | 232119_at | 1.00E−04 | SYNPO2 |
| 5016 | 232445_at | 0.002575 | SYT9 |
| 5736 | 1559289_at | 3.77E−04 | TADA1 |
| 2985 | 209358_at | 1.00E−06 | TAF11 |
| 4291 | 214690_at | 0.002 | TAF1B |
| 5043 | 1553011_at | 4.63E−04 | TAF1L |
| 2824 | 1556178_x_at | 3.49E−04 | TAF8 |
| 3676 | 1556176_at | 6.77E−04 | TAF8 |
| 4793 | 234697_x_at | 3.59E−04 | TAMM41 |
| 3588 | 216310_at | 2.62E−04 | TAOK1 |
| 1043 | 214413_at | 1.49E−04 | TAT |
| 1043 | 206916_x_at | 1.39E−04 | TAT |
| 6358 | 1569566_at | 1.40E−07 | TBC1D1 |
| 8156 | 202495_at | 2.23E−04 | TBCC |
| 5736 | 206451_at | 1.74E−04 | TBCCD1 |
| 663 | 1570293_at | 7.75E−04 | TBL1X |
| 8306 | 1564683_at | 3.45E−04 | TBL3 |
| 8015 | 207662_at | 5.44E−04 | TBX1 |
| 1043 | 215122_at | 5.01E−04 | TBX6 |
| 5873 | 207554_x_at | 6.87E−05 | TBXA2R |
| 2089 | 211590_x_at | 9.17E−06 | TBXA2R |
| 8324 | 1566207_at | 8.90E−04 | TCEA1 |
| 483 | 1566208_at | 2.33E−04 | TCEA1 |
| 3548 | 1563863_x_at | 3.00E−06 | TCEANC |
| 3548 | 1563862_at | 3.00E−06 | TCEANC |
| 5170 | 1562470_at | 0.001288 | TCEANC2 |
| 5328 | 205255_x_at | 1.38E−04 | TCF7 |
| 5328 | 220826_at | 5.61E−05 | TCP10L |
| 5937 | 206438_x_at | 5.68E−04 | TCTN2 |
| 3620 | 1569392_at | 2.32E−04 | TECR |
| 4482 | 241961_at | 3.00E−08 | TECRL |
| 5170 | 215673_at | 2.73E−04 | TEF |
| 185 | 219724_s_at | 1.52E−04 | TESPA1 |
| 324 | 1566606_a_at | 5.15E−05 | TEX9 |
| 5016 | 1566932_x_at | 0.002033 | TFB2M |
| 5115 | 207385_at | 1.00E−04 | TFDP3 |
| 3592 | 215863_at | 4.93E−04 | TFR2 |
| 6366 | 203084_at | 7.00E−07 | TGFB1 |
| 9642 | 1566901_at | 2.00E−05 | TGIF1 |
| 2824 | 1554608_at | 2.78E−04 | TGOLN2 |
| 7992 | 219231_at | 7.59E−04 | TGS1 |
| 1407 | 1566888_at | 6.80E−04 | THADA |
| 5796 | 244190_at | 0.003 | THAP5 |
| 7331 | 235218_x_at | 6.38E−04 | THAP6 |
| 2824 | 1562490_at | 1.57E−04 | THEM5 |
| 9475 | 242377_x_at | 3.29E−04 | THUMPD3 |
| 8010 | 215455_at | 5.27E−04 | TIMELESS |
| 5170 | 215168_at | 9.76E−04 | TIMM17A |
| 5016 | 219058_x_at | 4.93E−04 | TINAGL1 |
| 3635 | 239796_x_at | 0.002 | TIRAP |
| 7964 | 1557275_a_at | 3.03E−04 | TLCD2 |
| 2824 | 241642_x_at | 1.62E−04 | TLK1 |
| 2824 | 241643_at | 2.10E−05 | TLK1 |
| 5170 | 238168_at | 5.99E−05 | TM4SF1 |
| 7313 | 217974_at | 2.18E−04 | TM7SF3 |
| 2113 | 1563222_at | 4.46E−04 | TMBIM4 |
| 4808 | 219206_x_at | 5.61E−05 | TMBIM4 |
| 2985 | 242644_at | 1.00E−06 | TMC8 |
| 8943 | 208715_at | 6.00E−05 | TMCO1 |
| 4793 | 233399_x_at | 3.11E−05 | TMED10P1 /// ZNF252 |
| 9573 | 1558053_s_at | 5.23E−04 | TMED4 |
| 2582 | 239283_at | 2.00E−04 | TMED5 |
| 5016 | 1552302_at | 0.0016 | TMEM106A |
| 2274 | 1558712_at | 1.60E−04 | TMEM134 |
| 9628 | 219589_s_at | 4.83E−05 | TMEM143 |
| 4579 | 231923_at | 8.86E−04 | TMEM150C |
| 5937 | 222267_at | 1.68E−04 | TMEM209 |
| 1428 | 239593_at | 8.05E−04 | TMEM213 |
| 2497 | 220934_s_at | 0.0011 | TMEM223 |
| 6249 | 244495_x_at | 9.43E−04 | TMEM241 |
| 5170 | 224493_x_at | 5.97E−04 | TMEM241 |
| 2045 | 219503_s_at | 1.00E−05 | TMEM40 |
| 5328 | 235037_at | 5.73E−05 | TMEM41A |
| 9396 | 231696_x_at | 0.002 | TMEM50B |
| 2824 | 222907_x_at | 1.24E−04 | TMEM50B |
| 6882 | 1552765_x_at | 7.00E−05 | TMEM67 |
| 6882 | 1552763_at | 7.00E−05 | TMEM67 |
| 2650 | 1569377_at | 3.00E−05 | TMEM67 |
| 9338 | 226483_at | 5.54E−05 | TMEM68 |
| 524 | 223335_at | 3.37E−04 | TMEM69 |
| 9475 | 223771_at | 7.70E−04 | TMEM87A |
| 5170 | 244716_x_at | 3.26E−04 | TMIGD2 |
| 6776 | 221032_s_at | 0.003 | TMPRSS5 |
| 559 | 1555707_at | 6.74E−04 | TNAP |
| 2824 | 1552648_a_at | 9.78E−06 | TNFRSF10A |
| 9628 | 231775_at | 1.84E−04 | TNFRSF10A |
| 5731 | 203508_at | 0.002 | TNFRSF1B |
| 4268 | 207536_s_at | 2.76E−04 | TNFRSF9 |
| 3348 | 214329_x_at | 3.00E−06 | TNFSF10 |
| 10051 | 233935_at | 1.00E−04 | TNFSF14 |
| 5328 | 207907_at | 7.46E−05 | TNFSF14 |
| 4268 | 221085_at | 1.28E−04 | TNFSF15 |
| 9397 | 220655_at | 2.89E−04 | TNIP3 |
| 9338 | 216439_at | 1.43E−04 | TNK2 |
| 1043 | 215844_at | 2.77E−05 | TNPO2 |
| 492 | 222265_at | 7.67E−04 | TNS4 |
| 8426 | 1566833_x_at | 0.0015 | TOP1P2 |
| 8426 | 1566832_at | 0.0015 | TOP1P2 |
| 9365 | 237469_at | 0.001436 | TOP2A |
| 9338 | 1554849_at | 1.12E−04 | TOR1A |
| 5328 | 219620_x_at | 7.52E−05 | TOR4A |
| 5328 | 233589_x_at | 9.71E−05 | TOR4A |
| 5618 | 201746_at | 7.29E−04 | TP53 |
| 5170 | 220403_s_at | 2.04E−04 | TP53AIP1 |
| 1177 | 235192_at | 0.00251 | TP53RK |
| 8766 | 1569502_s_at | 8.81E−05 | TP73-AS1 |
| 9345 | 214601_at | 1.23E−04 | TPH1 |
| 6067 | 222976_s_at | 0.002 | TPM3 |
| 5732 | 203672_x_at | 1.35E−04 | TPMT |
| 492 | 1569856_at | 3.20E−04 | TPP2 |
| 2824 | 1562566_at | 0.001012 | TRAF3IP2-AS1 |
| 5328 | 243553_x_at | 1.19E−04 | TRAF3IP2-AS1 |
| 4936 | 215269_at | 0.003 | TRAPPC10 |
| 10065 | 213193_x_at | 0.002 | TRBC1 |
| 492 | 203148_s_at | 8.96E−04 | TRIM14 |
| 9628 | 204341_at | 1.66E−04 | TRIM16 |
| 5328 | 214815_at | 5.74E−05 | TRIM33 |
| 1043 | 203568_s_at | 2.18E−04 | TRIM38 |
| 3465 | 235084_x_at | 3.67E−04 | TRIM38 |
| 6358 | 224159_x_at | 1.20E−07 | TRIM4 |
| 796 | 210705_s_at | 3.19E−05 | TRIM5 |
| 264 | 231876_at | 0.001075 | TRIM56 |
| 9628 | 235081_x_at | 7.42E−04 | TRIM65 |
| 6844 | 1554182_at | 2.00E−05 | TRIM73 /// TRIM74 |
| 2824 | 210760_x_at | 1.89E−04 | TRIP11 |
| 5873 | 240166_x_at | 2.34E−05 | TRMT10B |
| 1562 | 235097_at | 9.25E−04 | TRMT44 |
| 6520 | 223109_at | 4.00E−06 | TRUB2 |
| 7331 | 244391_at | 7.93E−05 | TSEN2 |
| 5732 | 203227_s_at | 2.40E−04 | TSPAN31 |
| 9707 | 209109_s_at | 3.00E−06 | TSPAN6 |
| 5328 | 218155_x_at | 1.13E−04 | TSR1 |
| 2824 | 1557073_s_at | 3.07E−04 | TTBK2 |
| 416 | 215849_x_at | 2.79E−04 | TTC18 |
| 10019 | 235651_at | 9.12E−04 | TTC22 |
| 2395 | 244189_at | 0.003 | TTC28-AS1 |
| 2824 | 216768_x_at | 9.69E−05 | TTC38 |
| 3588 | 1555307_at | 2.33E−06 | TTF2 |
| 3465 | 208099_x_at | 6.87E−04 | TTLL5 |
| 5316 | 210389_x_at | 1.63E−05 | TUBD1 |
| 5016 | 227967_at | 0.002314 | TUBGCP5 |
| 9365 | 227873_at | 0.00275 | TXNDC15 |
| 416 | 1561080_at | 1.50E−04 | TXNRD1 |
| 7567 | 217497_at | 3.00E−04 | TYMP |
| 6698 | 241825_at | 5.08E−05 | TYW5 |
| 5328 | 231904_at | 3.40E−04 | U2AF1 |
| 5043 | 236715_x_at | 2.75E−04 | UACA |
| 3620 | 229587_at | 1.78E−04 | UBA2 |
| 3465 | 228353_x_at | 1.39E−04 | UBASH3B |
| 3402 | 202335_s_at | 0.001682 | UBE2B |
| 4317 | 238958_at | 0.003333 | UBE2E1 |
| 2824 | 233360_at | 4.05E−04 | UBE2I |
| 5043 | 211008_s_at | 1.44E−04 | UBE2I |
| 9110 | 225209_s_at | 0.004 | UBE2J2 |
| 4808 | 222252_x_at | 2.80E−04 | UBQLN4 |
| 2824 | 215636_at | 3.18E−04 | UBR4 |
| 2824 | 235327_x_at | 6.07E−05 | UBXN2A |
| 5016 | 218050_at | 0.001174 | UFM1 |
| 7649 | 216687_x_at | 4.32E−05 | UGT2B15 |
| 8791 | 207392_x_at | 7.00E−06 | UGT2B15 |
| 5170 | 1568789_at | 0.001391 | UHRF1BP1 |
| 3993 | 221323_at | 1.02E−04 | ULBP1 |
| 5328 | 202707_at | 1.03E−04 | UMPS |
| 5170 | 214817_at | 5.91E−04 | UNC13A |
| 5328 | 207499_x_at | 4.60E−04 | UNC45A |
| 7567 | 221064_s_at | 1.39E−04 | UNKL |
| 2824 | 210065_s_at | 7.07E−05 | UPK1B |
| 4268 | 223613_at | 1.00E−04 | UQCR11 |
| 5328 | 201671_x_at | 8.21E−05 | USP14 |
| 5328 | 216957_at | 1.04E−04 | USP22 |
| 6811 | 230623_x_at | 0.0012 | USP28 |
| 9628 | 207365_x_at | 5.88E−04 | USP34 |
| 7752 | 232621_at | 0.001128 | USP48 |
| 4331 | 216775_at | 9.30E−05 | USP53 |
| 4579 | 208067_x_at | 2.54E−04 | UTY |
| 5043 | 207263_x_at | 1.63E−04 | VEZT |
| 5170 | 1554388_at | 5.18E−04 | VPS29 |
| 1732 | 204590_x_at | 4.89E−04 | VPS33A |
| 5170 | 223845_at | 5.75E−04 | VSIG10 |
| 559 | 231770_x_at | 6.70E−04 | WDPCP |
| 7645 | 241919_x_at | 0.00113 | WDR31 |
| 5737 | 238677_at | 6.73E−04 | WDR36 |
| 5316 | 233242_at | 0.00325 | WDR73 |
| 492 | 205519_at | 3.12E−04 | WDR76 |
| 3796 | 238650_x_at | 3.00E−06 | WDR89 |
| 5736 | 1560662_s_at | 1.80E−04 | WHAMMP2 /// WHAMMP3 |
| 5170 | 224185_at | 3.93E−04 | WRAP53 |
| 941 | 241626_at | 1.00E−05 | WTAP |
| 5736 | 241950_at | 3.63E−04 | WWC1 |
| 285 | 228617_at | 4.00E−05 | XAF1 |
| 9658 | 206133_at | 6.00E−04 | XAF1 |
| 9365 | 242021_at | 0.0028 | XBP1 |
| 5328 | 220020_at | 2.65E−04 | XPNPEP3 |
| 4268 | 226891_at | 2.15E−05 | XXYLT1 |
| 8699 | 1570528_at | 1.89E−04 | XYLT2 |
| 10019 | 210917_at | 3.50E−04 | YES1 |
| 9345 | 1554553_s_at | 7.64E−06 | YIF1B |
| 6071 | 223519_at | 1.00E−05 | ZAK |
| 4268 | 1555259_at | 0.001302 | ZAK |
| 5328 | 208087_s_at | 1.45E−05 | ZBP1 |
| 10019 | 1557036_at | 4.11E−04 | ZBTB1 |
| 2824 | 220391_at | 2.94E−04 | ZBTB3 |
| 9224 | 225845_at | 6.00E−04 | ZBTB44 |
| 5731 | 216844_at | 8.35E−04 | ZC3H7B |
| 9381 | 206169_x_at | 2.00E−06 | ZC3H7B |
| 1697 | 225634_at | 0.002 | ZC3HAV1 |
| 2824 | 220104_at | 4.31E−05 | ZC3HAV1 |
| 5170 | 1553781_at | 5.52E−04 | ZC3HAV1L |
| 2824 | 232911_at | 1.27E−04 | ZFP14 |
| 1732 | 207090_x_at | 4.57E−05 | ZFP30 |
| 6386 | 243161_x_at | 0.002 | ZFP42 |
| 345 | 1554777_at | 3.33E−04 | ZFP42 |
| 9352 | 235176_at | 3.50E−04 | ZFP82 |
| 8849 | 232774_x_at | 2.10E−05 | ZIK1 |
| 3592 | 1553022_at | 9.34E−05 | ZIM3 |
| 2045 | 1562305_x_at | 2.34E−04 | ZKSCAN3 |
| 8847 | 216350_s_at | 1.00E−04 | ZNF10 |
| 2824 | 1552947_x_at | 3.22E−04 | ZNF114 |
| 2824 | 1552946_at | 3.22E−04 | ZNF114 |
| 6358 | 234394_at | 4.00E−08 | ZNF124 |
| 4579 | 219854_at | 3.92E−04 | ZNF14 |
| 2396 | 235580_at | 0.001 | ZNF141 |
| 8850 | 216677_at | 4.00E−05 | ZNF154 |
| 5937 | 214715_x_at | 1.45E−04 | ZNF160 |
| 2594 | 1567032_s_at | 4.00E−06 | ZNF160 |
| 2594 | 1567031_at | 4.00E−06 | ZNF160 |
| 8179 | 206683_at | 4.00E−06 | ZNF165 |
| 8614 | 207417_s_at | 5.01E−06 | ZNF177 /// ZNF559-ZNF177 |
| 8017 | 234953_x_at | 1.00E−05 | ZNF19 |
| 5736 | 231848_x_at | 1.33E−04 | ZNF207 |
| 3609 | 238772_at | 3.01E−04 | ZNF207 |
| 9628 | 239937_at | 1.45E−04 | ZNF207 |
| 4579 | 1555510_at | 9.97E−05 | ZNF215 |
| 8270 | 220847_x_at | 6.45E−04 | ZNF221 |
| 8812 | 206175_x_at | 1.00E−06 | ZNF222 |
| 8812 | 220019_s_at | 1.00E−06 | ZNF224 |
| 8812 | 233461_x_at | 1.00E−06 | ZNF226 |
| 8845 | 217403_s_at | 5.05E−06 | ZNF227 |
| 3102 | 219123_at | 4.00E−06 | ZNF232 |
| 9475 | 210595_at | 0.001099 | ZNF235 |
| 2594 | 203247_s_at | 6.00E−04 | ZNF24 |
| 2824 | 1569557_at | 1.75E−04 | ZNF248 |
| 7214 | 206900_x_at | 7.27E−04 | ZNF253 |
| 8849 | 223714_at | 2.67E−06 | ZNF256 |
| 1043 | 216710_x_at | 2.55E−04 | ZNF287 |
| 8614 | 1567856_x_at | 0.003 | ZNF29P |
| 8614 | 1567855_at | 0.003 | ZNF29P |
| 2594 | 244289_at | 6.00E−04 | ZNF300P1 |
| 2594 | 236551_at | 5.00E−05 | ZNF311 |
| 2594 | 236550_s_at | 5.00E−05 | ZNF311 |
| 9345 | 1558888_x_at | 2.76E−05 | ZNF321P /// ZNF816 /// ZNF816-ZNF321P |
| 3677 | 1558887_at | 4.00E−07 | ZNF321P /// ZNF816 /// ZNF816-ZNF321P |
| 492 | 219765_at | 6.50E−04 | ZNF329 |
| 3102 | 219228_at | 0.003 | ZNF331 |
| 6493 | 1552375_at | 6.00E−05 | ZNF333 |
| 173 | 220022_at | 0.00102 | ZNF334 |
| 7992 | 232873_at | 0.001588 | ZNF33A |
| 492 | 1558586_at | 1.82E−04 | ZNF33B |
| 2262 | 240449_at | 0.003 | ZNF341 |
| 663 | 242208_at | 4.02E−05 | ZNF37BP |
| 8156 | 1554985_at | 1.01E−04 | ZNF396 |
| 1659 | 219826_at | 0.003 | ZNF419 |
| 5736 | 222136_x_at | 2.50E−04 | ZNF43 |
| 5328 | 206829_x_at | 1.46E−05 | ZNF430 |
| 4878 | 233712_at | 5.00E−04 | ZNF44 |
| 5328 | 215359_x_at | 6.45E−04 | ZNF44 |
| 483 | 215892_at | 2.53E−04 | ZNF440 |
| 8614 | 208081_s_at | 0.003 | ZNF442 |
| 5937 | 233992_x_at | 1.56E−04 | ZNF445 |
| 8813 | 207304_at | 5.00E−05 | ZNF45 |
| 6249 | 233175_at | 0.001279 | ZNF460 |
| 1047 | 232391_at | 7.30E−04 | ZNF461 |
| 8614 | 211923_s_at | 7.00E−06 | ZNF471 |
| 8850 | 213124_at | 1.00E−05 | ZNF473 |
| 8614 | 1555368_x_at | 0.001 | ZNF479 |
| 8614 | 1555367_at | 0.001 | ZNF479 |
| 1562 | 1552427_at | 6.28E−04 | ZNF485 |
| 2824 | 1553901_x_at | 4.50E−04 | ZNF486 |
| 5736 | 231988_x_at | 1.79E−04 | ZNF490 |
| 7649 | 235604_x_at | 1.00E−05 | ZNF493 |
| 8311 | 211064_at | 8.71E−05 | ZNF493 |
| 3102 | 225333_at | 0.003 | ZNF496 |
| 9628 | 225335_at | 4.57E−04 | ZNF496 |
| 5328 | 1562386_s_at | 1.46E−05 | ZNF501 |
| 5170 | 221626_at | 2.49E−04 | ZNF506 |
| 1444 | 203604_at | 3.53E−04 | ZNF516 |
| 8766 | 215019_x_at | 2.00E−04 | ZNF528 |
| 8890 | 235121_at | 1.00E−12 | ZNF542 |
| 10019 | 218735_s_at | 8.92E−04 | ZNF544 |
| 1659 | 1558622_a_at | 5.00E−06 | ZNF548 |
| 3102 | 215315_at | 0.001515 | ZNF549 |
| 8849 | 242992_at | 3.50E−06 | ZNF551 |
| 5736 | 219741_x_at | 5.45E−04 | ZNF552 |
| 8614 | 1553286_at | 2.00E−05 | ZNF555 |
| 8708 | 220444_at | 8.00E−17 | ZNF557 |
| 447 | 219163_at | 1.50E−05 | ZNF562 |
| 2824 | 1552770_s_at | 5.24E−04 | ZNF563 |
| 8461 | 1564356_at | 4.88E−04 | ZNF568 |
| 8614 | 1568900_a_at | 3.50E−05 | ZNF568 |
| 8461 | 1552562_at | 0.001517 | ZNF570 |
| 7250 | 1558253_x_at | 2.50E−05 | ZNF587 /// ZNF587B |
| 7250 | 1558251_a_at | 2.50E−05 | ZNF587 /// ZNF587B |
| 5931 | 221963_x_at | 2.00E−07 | ZNF587B |
| 8699 | 1569108_a_at | 0.001362 | ZNF589 |
| 8847 | 242463_x_at | 2.00E−10 | ZNF600 |
| 2824 | 1559849_at | 1.25E−04 | ZNF605 |
| 5328 | 232303_at | 1.01E−04 | ZNF608 |
| 8461 | 235953_at | 6.50E−06 | ZNF610 |
| 6251 | 1559059_s_at | 1.67E−04 | ZNF611 |
| 2824 | 208137_x_at | 2.13E−04 | ZNF611 |
| 6844 | 210599_at | 2.00E−05 | ZNF614 |
| 5170 | 1558743_at | 3.32E−04 | ZNF620 |
| 3592 | 206188_at | 4.24E−05 | ZNF623 |
| 3414 | 1552769_at | 3.00E−06 | ZNF625 |
| 2824 | 1559616_x_at | 4.64E−04 | ZNF626 |
| 4579 | 239243_at | 3.81E−06 | ZNF638 |
| 8763 | 218413_s_at | 3.03E−04 | ZNF639 |
| 8846 | 223616_at | 8.00E−04 | ZNF649 |
| 5328 | 220760_x_at | 3.63E−05 | ZNF665 |
| 4793 | 220215_at | 2.92E−05 | ZNF669 |
| 173 | 208273_at | 3.00E−06 | ZNF670 /// ZNF670-ZNF695 /// ZNF695 |
| 8461 | 1554465_s_at | 0.002 | ZNF673 /// ZNF674 |
| 8303 | 215309_at | 0.002 | ZNF674 |
| 8813 | 217547_x_at | 2.00E−04 | ZNF675 |
| 5043 | 234920_at | 2.62E−04 | ZNF7 |
| 1667 | 220242_x_at | 4.00E−05 | ZNF701 |
| 10009 | 206557_at | 5.00E−04 | ZNF702P |
| 8195 | 239482_x_at | 0.002667 | ZNF708 |
| 8851 | 233754_x_at | 0.002 | ZNF71 |
| 7331 | 215978_x_at | 9.45E−04 | ZNF721 |
| 8461 | 1559275_x_at | 4.42E−04 | ZNF732 |
| 8461 | 1559274_at | 4.42E−04 | ZNF732 |
| 559 | 205881_at | 7.77E−04 | ZNF74 |
| 6251 | 206180_x_at | 1.74E−04 | ZNF747 |
| 8319 | 242633_x_at | 2.96E−04 | ZNF75A |
| 416 | 1558755_x_at | 3.81E−04 | ZNF763 |
| 416 | 1558754_at | 3.81E−04 | ZNF763 |
| 5328 | 219782_s_at | 1.44E−04 | ZNF771 |
| 8852 | 230517_at | 0.00125 | ZNF775 |
| 173 | 1559789_a_at | 1.00E−05 | ZNF781 |
| 8162 | 1554770_x_at | 1.00E−05 | ZNF785 |
| 1885 | 239083_at | 0.001055 | ZNF786 |
| 3102 | 216486_x_at | 0.003 | ZNF79 |
| 3102 | 216482_x_at | 0.003 | ZNF79 |
| 5328 | 1553704_x_at | 4.26E−04 | ZNF791 |
| 5328 | 1553703_at | 4.26E−04 | ZNF791 |
| 2824 | 1554476_x_at | 1.72E−04 | ZNF808 |
| 3102 | 215901_at | 0.001033 | ZNF81 |
| 9628 | 1561038_at | 1.03E−04 | ZNF81 |
| 10019 | 217541_x_at | 1.16E−04 | ZNF816 /// ZNF816-ZNF321P |
| 5115 | 226315_at | 5.00E−04 | ZNF830 |
| 2824 | 215826_x_at | 2.06E−04 | ZNF835 |
| 5618 | 1569076_a_at | 0.0025 | ZNF836 |
| 8851 | 241277_x_at | 3.00E−06 | ZNF837 |
| 542 | 1555078_at | 2.00E−07 | ZNF843 |
| 5043 | 1564362_x_at | 7.39E−05 | ZNF843 |
| 8717 | 1569157_s_at | 0.003 | ZNF846 |
| 9352 | 1564331_at | 2.95E−05 | ZNF846 |
| 2594 | 1564662_at | 6.00E−04 | ZNF852 |
| 345 | 235913_at | 3.28E−05 | ZNF880 |
| 6249 | 1569277_at | 0.001012 | ZNF91 |
| 8614 | 206059_at | 0.001 | ZNF91 |
| 2594 | 215758_x_at | 5.00E−05 | ZNF93 |
| 3609 | 215985_at | 1.50E−04 | ZNRD1-AS1 |
| 3857 | 224124_at | 0.001 | ZRANB3 |
| 8845 | 219904_at | 7.00E−04 | ZSCAN5A |
| 1129 | 235448_at | 4.63E−06 | ZXDC |

^{A}shows the SEQ ID NO;
^{B}affymetrix probe ID that harbors significant identity with the repeats from (A);
^{C}shows the significance of the identity;
^{D}shows the transcript that the affymetrix probe detects

For the probes presented in Table 6 and 7 a large amount of data is available at http://www.ncbi.nlm.nih.gov/pubmed/geoprofiles, revealing their roles in disease (See List 1 below). Further, the site provides information on gene regulation upon exposure to (a)biotic compounds or medicine (See List 2 below). Unfortunately, from this data it remains unclear whether the effect is obtained through the human CRISPRs being switched ON/OFF upon exposure to (a)biotic compounds or specific pharmaceutical compounds. In order to obtain more information on the mechanism of the observed effects, the exact positions on the Hg19 genome from which the Human Genome U133 Plus 2.0 Array probe sequences were obtained that were used for the above described BLAST analysis were available. Using the Galaxy software package for interval analysis and the exact positions on the Hg19 genome for the human CRISPRs we were able to identify twenty two human CRISPRs that exactly matched the Human Genome U133 Plus 2.0 Array probes. In FIG. 7 expression examples are provided of four probes that were genome position matched to a human CRISPR and resided in an intergenic region. Seventy-four occasions in total were obtained for these four probes demonstrating that a human CRISPR is more active or less active upon (a)biotic or medicine exposure compared to the corresponding control(s) (FIG. 7).

List 1) shows in a repetitive manner in fasta format the CRISPR SEQ ID NO, the Affymetrix probe ID that harbored a significant identity with the concatenated CRISPR SEQ ID followed by a list of diseases that are linked to the Affymetrix probes.

List 2) shows in a repetitive manner in fasta format the CRISPR SEQ ID NO, the Affymetrix probe ID that harbored a significant E-value with the repeat of the connected CRISPR SEQ ID to this probe after BLAST analyses followed by a list of (combination of) drugs that have significant effect on the transcript(s) that this Affymetrix probe recognizes.

List 3) is connected to Table 4 and shows in a repetitive number for each row in the table (row no. is indicated) and shows the genes involved in the disease targets specified in Table 4.

List 1

>5927 1562661 at
(A)biotic stress effect on eukaryotic cells, Pancreatic ductal adenocarcinoma, Streptococcus
oneumoniae infection, Tendinopathy, Medicine resistance cancer cells, Staphylococcus aureus
infection, Prostate cancer, Erythroid differentiation, Pancreatic cancer, Adenoid cystic carcinoma,
Systemic lupus erythematosus, Metaphase II stage oocytes maturation, Pediatric immune
thrombocytopenia
>4064 239410 at
(A)biotic stress effect on eukaryotic cells, Erythroid differentiation, Melanoma progression,
Embryonic development, Breast cancer, Influenza virus infection, Endometrium and menstrual cycle,
Systemic lupus erythematosus, Cystic fibrosis, Francisella tularensis infections, Autosomal dominant
monocytopenia, Polycystic ovary syndrome, Obesity, Papillary thyroid cancer, Newly diagnosed and
chronic pediatric immune chrombocytopenia, Inflammatory dilated cardiomyopathy, Medicine resistance
cancer cells, Teratozoospermia, Muscle function and aging, Male Fetal and adult reticulocytes
>7250 210230 at
(A)biotic stress effect on eukaryotic cells, Essential thrombocythemia, Pituitary adenoma subtypes,
Lassa tever, Multiple sclerosis, Hutchinson-Giltord progeria syndrome, Tuberculosis, Tibial muscular
dystrophy, Endometriosis, Sindbis virus infection, Essential thrombocythemia, Prostate cancer,
Embryonic development, Crohns disease, Small cell lung cancers, Sjögren's syndrome, Alzheimer's
disease, Colon cancer, Rheumatoid arthritis, Toxoplasma gondii infecticn, Non-melanoma skin cancer,
Heart failure, Cystic fibrosis, Prostate cancer, Brain cancer, Pediatric glioblastoma brain tumors,
Francisella tularensis infections, Lethal congenital contracture syndrome, Streptococcus gordonii
infection, T-cell acute lymphoblastic leukemia, Porphyromonas gingivalis infection, Autosomal
dominant monocytopenia, Metaphase II stage oocytes maturation, Acute myeloid leukemia, Muscle
function and aging, Familial combined hyperlipidemia, Breast cancer, House dust mite allergy,
Diamond-Blackfan anaemia, Pulmonary sarcoidosis, Medicine resistance cancer cells, COPD, Sex
specific transcription in hypothalamus
>9629 234590 x at
(A)biotic stress effect on eukaryotic cells, Eutchinson-Gilford progeria syndrome, High-grade
gliomas, Idiopathic pulmonary fibrosis, AIDS-related lymphomas, Multiple sclerosis, Systemic juvenile
idiopathic arthritis, Cerebral aging and Alzheimer's disease, Embryonic development, Burkitt lymphoma
subtypes, Multiple sclerosis, Duchenne Muscular, Dystrophy, Influenza virus infection, Prostate
cancer, Familial hypercholesterolemia, Teratozoospermia, Optineurin depletion, Aldosterone-producing
adeonoma, Dystocic myometrium, Pancreatic cancer, Anaplasma phagocytophilum infection, Systemic lupus
erythematosus, Fetal and adult reticulocytes, Medicine resistance cancer cells
>4599 238016 s at
(A)biotic stress effect on eukaryotic cells Medicine resistance cancer cells, Pompe disease,
Polycystic ovary syndrome, Obesity, Teratozoospermia, ISCU myopathy, Schizophrenia, Colorectal
cancer, Endometriosis, Metaphase II stage oocytes maturation, Optineurin depletion, Vulvar
intraepithelial neoplasia, Pancreatic Tumor Breast cancer, Pediatric glioblastoma brain tumors,
Cystic fibrosis, Heart failure, Kaposi's sarcoma, Sjögren's syndrome, Duchenne, Muscular Dystrophy
response to oxandrolone, Prechondrocyte differentiation, Erythroid differentiation, HIV-associated
neurocognitive disorders, Prostate cancer, Psoriasis, Rhinovirus infection, Plasmodium infection,
Systemic juvenile idiopathic arthritis and non-systemic JIA subtypes, Coronary artery disease,
Multiple sclerosis, Medulloblastomas, Alzheimer's disease
>6399 232273 at
(A)biotic stress effect on eukaryotic cells, Notch-on/off effects on T-lymphoblastic leukemia cells,
Atopic dermatitis, Nodular lymphocyte-predominant, Hodgkin lymphoma, Glioblastoma, Psoriasis, HIV-
associated neurocognitive disorders, Endometrium and menstrual cycle, Staphylococcus superantigen
exposure, Inflammatory bowel disease, Duchenne, Muscular Dystrophy, Sjögren's syndrome, HPV16
infection, Prostate cancer, Placentae development, Chromophobe renal cell carcinoma, ISCU myopathy,
Pulmonary sarcoidosis, Inflammatory dilated cardiomyopathy, Colorectal cancer, Alcoholic hepatitis,
Chronic B-lymphocytic leukemia, Metachronous liver metastases, Medicine resistance cancer cells,
Chlamydia pneumonia infection, Metaphase II stage oocytes maturation, Muscle function and aging
>8195 230580 at
(A)biotic stress effect on eukaryotic cells, Obesity, Asthma, Stage I endometrial cancer, Age and
skeletal muscle types, Prostate cancer, Ovarian endometriosis, Hutchinson-Gilford progeria syndrome,
Polycystic ovary syndrome, Plasmodium infection, Respiratory syncytial virus infection,
Cryptosporidium infection, Erythroid differentiation, Embryonic development, Duchenne, Muscular
Dystrophy Influenza virus infection, Inflammatory bowel disease, Cystic fibrosis, HPV16 infection,
Francisella tularensis infection, Ductal carcinoma in situ, Pancreatic cancer, Multiple sclerosis,
Muscle function and aging - female, Asthma,
Fetal and adult reticulocytes, Medicine resistance cancer cells
>2750 1569527 at
Alcoholic hepatitis, Hereditary gingival fibromatosis, Rotavirus infection, AIDS-related lymphomas,
Hepatitis C, Virus infection, Ulcerative colitis, Williams-Beuren Syndrome, Nodular Lymphocyte-
predominant, Hodgkin lymphoma, Tibial muscular dystrophy, HBV-associated, ALF, Colorectal cancer
primary or metastatic lesions, Erythroid differentiation, HIV-associated neurocognitive disorders,
Multiple sclerosis, Embryonic development, Multistep pancreatic carcinogenesis, Polycystic ovary
syndrome, Sjögren's syndrome, Obesity, Pulmonary sarcoidosis, Multiple sclerosis, Endometriosis,
Chronic B-lymphocytic leukemia, Vulvar intraepithelial neoplasia, Asthma, Autosomal dominant
monocytopenia
(A)biotic stress effect on eukaryotic cells, Medicine resistance cancer cells, Multiple myeloma
>5856 1566501 at
Autosomal dominant monocytopenia, Atopic dermatitis, Tendinopathy, (A)biotic stress effect on
eukaryotic cells, HBV-associated, ALF, Unresectable colorectal cancer, Prostate cancer, Radiation
and leukemia, Multistep pancreatic carcinogenesis, Familia 1 hypercholesterolemia and
atherosclerosis, Francisella infection, Systemic lupus erythematosas, Endometriosis, Nephroblastoma,
Asthma, Obesity, Polycystic ovary syndrome, Pediatric acute leukemia, Liver transplantation
tolerance, Medicine resistance cancer cells, Multiple sclerosis
>3595 1557580 at
Chronic B-lymphocytic leukemia, Acute myeloid leukemia, Systemic javenile idiopathic arthritis,
Tuberculosis, Ovarian endometriosis, Papillary thyroid carcinoma, HBV-associated, ALF, Tibial
muscular dystrophy, Respiratory syncytial virus infection, Embryonic development, Adenoid cystic
carcinoma, Sjögren's syndrome, Heart failure, Prostate cancer, Sonic hedgehog homolog,
Teratozoospermia, Staphylococcus aureus infection, Systemic lupus erythematosus, Inflammatory dilated
cardiomyopathy, Intracranial arterial aneurysm, Asthma, Metaphase II stage oocytes maturation, Liver
transplantation tolerance, Pulmonary sarcoidosis (A)biotic stress effect on eukaryotic cells,
Medicine resistance
cancer cells,
>9831 234753 x at
Chronic B-lymphocytic leukemia, Clear cell renal cell carcinoma, Ovarian endometriosis, Leishmania
major infection, (A)biotic stress effect on eukaryotic cells, Medicine resistance cancer cells,
Tibial muscular dystrophy, Embryonic development, Sjögren's syndrome, Cystic fibrosis, Multiple
sclerosis, Obesity, Synchronous and metachronous liver metestases, Anaplasma phagocytophilum
infection, Teratozoospermia, Francisella tularensis infections,
>3020 1563721 at
Colorectal cancer, House dust mite allergy, Preeclampsia, Rotavirus infection, Invasive ductal,
Multiple sclerosis, Alzheimer's
disease, Pituitary gonadotrope tumors, Respiratory syncytial
virus infection, Invasive ductal carcinoma, EPV16 infection, Multistep pancreatic carcinogenesis,
Pancreatic cancer
>4331 205183 at
Dystccic myometrium, Parkinson's disease, Measles, Rhinovirus infection, Essential thrombocythemia,
Cryptosporidium infection, Fetal lung development, Heart failure, RSV infection, Francisella
tularensis infections, Retinoblastoma, Dermatomyositis, ADH with Breast cancer, Anaplasma
phagocytophilum infaction, Neuroblastoma, Utcrinc fibroid, Glioblastoms pscudopalisading colls,
Retinoblastoma, Dermatomyositis, Muscle functioning and aging, Porphyromonas gingivalis, Kaposi
sarcoma, Inflammatory dilated cardiomyopathy, Early onset colorectal cancer, Staphylococcus aureus
infection, Chlamydia pneumonia infection, Chronic pediatric immune thrombocytopenia, Acute myelcid
leukemia, Obesity, Asthma atopic, Metaphase II stage oocytes maturation, Social isolation (A)biotic
stress effect on eukaryotic cells, Medicine resistance cancer cells
>8960 1560118 at
Growth factor effects on pancreatic adenocarcinoma, Metaphase II stage oocytes maturation,
Teratozoospermia, House dust mite allergy, Chlamydia pneumonia infection, Colorectal cancer,
Optineurin depletion, Medicine resistance cancer cells, Epithelium response to cigarette smoke and
estradiol, Dystocic myometrium, Intracranial arterial aneurysm, Gastric tubular adenocarcinoma,
Kaposi's sarcoma, HPV16 infection, Prostate cancer, Influenza virus infection, Aspergillus infection,
Acute myeloid
Leukemia, Cryptosporidium infection, Psoriasis, Plasmodium infection, Pituitary gonadotrope tumors,
Staphylococcus aureus infection, Alzheimer's Oisease
>3595 1566541 at
Head and neck squamous cell carcinoma tumors harboring HPV
(A)biotic stress effect on eukaryotic cells, Medicine resistance cancer cells, Aldosterone-producing
adeonoma, Colorectal cancer, Vulvar intraepithelial neoplasia, ISCU myopathy, Pulmonary sarcoidesis,
Hereditary gingival fibromatosis, House dust mite allergy, Autosomal dominant monocytopenia,
Optineurin depletion, Prechondrocytes, Heart failure, Prostate cancer, Influenza virus infection,
Aspergillus infection, Trypanosoma cruzi infection, Bipolar disorder, Embryonic development, Multiple
sclerosis, RSV infection, Inflammation vascular epithelial cells, Nodular lymphocyte-predominant,
Hodgkin lymphoma, Atopic dermatitis, Training resistance skeletal muscle, Tendinopathy,
Schizophrenia, Rotavirus infection
>8384 216692 at
Hereditary gingival fibromatosis (A)biotic stress effect on eukaryotic cells, Essential
thrombocythemia, Pancreatic ductal adenocarcinoma, Colorectal adenoma formation, Psoriasis, Type 2
diabetic, Chronic myelogenous leukemia, Fetal lung development, Melanocyte to melanoma
transformation, Prechondrocyte differentiation, T-cell prolymphocyzic leukemia, Preeclampsia B-cell
chronic lymphocytic leukemia, Sjögren's syndrome, Adenoid cystic carcinoma, Influenza virus
infection, Untreated juvenile dermatomyosites, Ewing family tumor, Familial combined hyperlipidemia,
Intracranial arterial aneurysm, Systemic lupus erythematosus, Dystocic myometrium, House dust mite
allergy, Liver transplantation tolerance, Chronic obstructive pulmonary disease, Medicine resistance
cancer cells, Anaplasma phagocytophilum infection Testosterone effect on skeletal muscles of HIV-
infected males,
>1038 1554963 at
Hereditary gingival fibromatosis, Teratozoospermia, Liver transplantation tolerance, Endometriosis,
Leishmania major infection, Multiple sclerosis, Plasmodium infection, Nodular lymphocyte-predominant,
Hodgkin lymphoma, Uveal melanoma, Influenza virus infection, Ulcerative colitis, Embryonic
development, Prechoncrocyte differentiation, Erythroid differentiation, Francisella tularensis
infections, Sjögren's syndrome, Colon cancer,
(A)biotic stress effect on eukaryotic cells, Newly diagnosed and chronic pediatric immune
thronbocytopenia, Medicine resistance cancer cells, Aldosterone-producing adeonoma, Autosomal
dominant monocytopenia, Systemic lupus erythematosus, Inflammatory dilated cardiomyopathy, Asthma
Obesity,
>185 1569182 at
Herpesvirus-8 infection, Preeclampsia, Pancreatic ductal adeno, Carcinoma, Alzheimer's disease,
Colorectal adenoma formation, Muscle training, Prostate cancer, Hepatitis C, Virus infection,
Cerebral aging, Tuberculosis, Endometriosis, Breast cancer, Adrenocortical adenomas, Influenza virus
infection, Multiple sclerosis, Prechondrocyte differentiation, Fetal lung development, Aldosterone-
producing adeonoma, Kaposi's sarcoma, Autosomal dominant monocytopenia, Optineurin depletion,
Papillary thyroid cancer, Teratozoospermia, Metaphase II stage oocytes maturation
(A)biotic stress effect on eukaryotic cells, Medicine resistance cancer cells,
>6399 243768 at
Human herpesvirus-8 infecfion, Influenza virus infection, Rheumatoid arthritis, Glioblastoma brain
tumors, Hepatitis C, Virus infection, Clear cell renal cell carcinoma, Ovarian endometriosis, Various
muscle diseases, Tuberculosis, Nodular lymphocyte-predominant, Hodgkin lymphoma, Williams-Beuren,
Syndrome, Asthma, Cibial muscular dystrophy, Plasmodium infection, Psoriasis, Embryonic developnbnt,
Erythroid differentiation, Fetal lung development, Duchenne, Muscular, Dystrophy, Multistep
pancreatic carcinogenesis, Inflammatory bowel disease, Tibolone hormone ettect on postmenopausal
endometrium, Hepatocellular carcinoma, Sjögren's syndrome, Prostate cancer, Familial
hypercholesterolemia and atherosclerosis, Aldosterone-producing adeonoma, Aging effect, HPSC,
Pituitary adenoma predisposition, Liver transplantation tolerance, Autosomal dominant monocytopenia,
Systemic lupus erythematosus, Synchronous and metachronous liver metastases, Early onset colorectal
cancer, Preeclamosia, Fetal and adult reticulocytes, Medulloblastoma, Obesity, Polycystic ovary
syndrome, Chronic pediatric inmune thrombocytopenia, Pulmonary sarcoidesis, Muscle function and aging
(A)biotic stress effect on eukaryotic cells, Medicine resistance cancer cells
>1038 1554964 x at
IGHV mutated/unmutated, Mantle, Cell, Lymphoma, Colorectal adenoma formation, Placental malaria,
Multiple sclerosis, Sjögren's syndrome, HBV-associated, ALF, Colon cancer cells, Multistep pancreatic
carcinogenesis, Francisella tularensis infection, Familial hypercholesterolemia and atherosclerosis,
Metaphase II stage oocytes maturation, Preeclampsia, Endometriosis, Chlamydia pneumonia infection,
Pituitary adenoma predisposition, Early onset colorectal cancer, House dust mite allergy, Liver
transplantation tolerance, Hereditary gingival fibromatosis, Infantile-onset, Pompe disease
(A)biotic stress effect on eukaryotic cells, Medicine resistance cancer cells
>8678 230461 s at
Inflammatory dilated cardiomyopathy, Prostate cancer, Colon cancer, Medicine resistance cancer cells,
Bipolar Disorder, Autophagy, Type II Diabetes, Hepatitis B virus, (HBV)-associated acute liver
failure, (ALF) patheots, Multiple Sclerosis, Interstitial lung disease
>8195 215958 at
Intracranial arterial aneurysm, Cryptosporidium infection, Pituitary adenoma subtypes, Essential
thrombocythemia, Alzheimer's disease, Colorectal adenoma formation, Placental malaria, Melanoma
progression, Hyperinsulinemia, (A)biotic stress effect on eukaryotic cells, Staphylococcus aureus
infection, Papillary thyroid carcinoma, HBV-associated, ALF, Hungington's disease, Ovarian cancer, B-
cell chronic lymphocytic leukemia, Prechondrocyte differentiation, Sjögren's syndrome, Prostate
cancor, Juvenilc dermatomyositis, Cystic fibrosis, HIV infection, Clioblastoma, Colorectal cancer,
Aldosterone-producing adeonoma, Dermatomyositis, Kaposi sarcoma, Porphyromonas gingivalis infection,
Medicine resistance cancer cells
>4331 1557335 at
Intracranial arterial aneurysm, Pancreatic ductal adenocarcinoma, Probiotic effect, Sonic hedgehog
homolog, Plasmodium infection, Prechondrocyte differentiation, Influenza virus infection, Obesity
(A)biotic stress effect on eukaryotic cells, Medicine resistance cancer cells, Cerebral
adrenoleukodystrophy,
>5856 234247 at
Leukemia, House dust if allergy, Atopic dermatitis, Multiple
Sclerosis, Renal cell carcinonh, Pituitary gonadotrope tumors, Psoriasis, Cystic fibrosis, Type 2
diabetes, Parkinson's disease, Myeloma, Prechondrocytes pancreatic cancer, Medicine resistance
cancer cells, Optineurin depletion, EPC response on exercise, Intracranial arterial aneurysm,
Metachronous liver metastases, HIV infection, Cushing's Syndrome, Sonic hedgehog homolog acute
myocardial infarctior, Heart failure, Aspergillus infection, Influenza infection, Lung cancer, Acute
myeloid leukemia, Endometrium and menstrual cycle, Embryonic development, Ovarian cancer,
Cryptosporidium infection, Prostate cancer
>2700 1556521 a at
Liver metastases, Heart failure, Type 2 diabetes, Plasmodium falciparum infection, HIV and SIV
infection, Breast cancer, Medicine resistance cancer cells, Optineurin depletion, Neural tube defect,
Metaphase II stage oocytes, Polycystic ovary syndrome, Vulvar intraepithelial neoplasia, Childhood
cerebral adrenoleukodystrophy
>3395 217482 at
Liver transplantation tolerance, Colorectal acenoma formation, SCTD, Acute myeloid leukemia,
Mitochondrial encephalomyopathies, Biomaterial engineering, Presymptomatic and symptomatic malaria,
Sarcoma, Ovarian endometriosis, Myelodysplastic syndrome, Dermatomyositis, Asthma, Prostate cancer,
HIV infection, Unresectable colorectal cancer, Erythroid differentiation, Ulcerative colitis,
Embryonic development, Fetal lung development, Influenza virus infection, Sjögren's syndrome,
Alzheimer's disease, Myelodysplastic syndromes, Scleroderma, Infant biliary atresia,
Teratozoospermia, Ductal carcinoma
(A)biotic stress effect on eukaryotic cells, Medicine resistance cancer cells, Polycystic ovary
syndrome
>63 1565857 at
Mantle Cell, Lymphoma, Intracranial arterial aneurysm, Nephroblastoma, Autosomal dominant
monocytopenia, Pulmonary sarcoidosis epithelium response cigarette smoke, Optineurin depletion,
Colorectal cancer, Hormone effect cancer cells, Chlamydia pneumonia infection, Tolerant renal
transplant recipients, HPV16 infection, Prostate cancer, HIV/SIV infection, Medicine resistance
cancer cells, Pancreatic carcinogenesis, Sjögren's syndrome, Aspergillus infection, Preeclampsia,
Embryonic development, Stress exposure skeletal muscle, HIV-associated neurocognitive disorders, Type
2 diabetes, Plasmodium falciparum infection, Parkinson's disease, Multiple sclerosis
>7988 1557774 at
Medicine resistance cancer cells, (A)biotic stress effect on eukaryotic cells, Intracrnial arterial
aneurysm, Polycystic ovary syndrome, Obesity, Teratozoospermia, Chronic B-lymphocytic leukemia,
Pulmonary sarcoidosis, Eosinophilic esophagitis, Staphylococcus aureus infection, Pancreatic, Tumor,
Colorectal cancer, Hereditary gingival fibromatosis, SIV virus infection cytokine effect on immune
cells, Pediatric immune thrombocytopenia, HPV16 virus infection, Prostate cancer, Kaposi's sarcoma,
Aspergillus infection, Hepatitis C virus infection, Muscle response to weight loss, HIV-associated
neurocognitive disorders, RSV virus infection, Psoriasis, Plasmodium infection, Prostate cancer,
Hepatitis B virus, (ALF), Burkitt lymphoma subtypes, Medulloblastoma, Malaria, Notch-on/off effects
oF T-lymphoblastic leukemia cells, Alzheimer's disease
>3595 1557581 x at
Medicine resistance cancer cells, Alzheimer's disease, Tuberculosis, Idiopathic pulmonary fibrosis,
Ovarian endometriosis, HIV-associated neurocognitive impairment, Acute myeloid leukemia, Obesity,
Tibial muscular dystrophy, Influenza virus infection, Neuroblastoma, Asthma, Prostate cancer,
Sjögren's syndrome, Colon cancer, Pulmonary sarcoidosis, Liver transplantation tolerance, Metaphase
II stage oocytes maturation (A)biotic stress effect on eukaryotic cells colecteral cancers,
>7665 234574 at
Medicine resistance cancer cells, Chronic B-lymphocytic leukemia, Transplantation resistance,
Multiple sclerosis, Obesity, Vitamine D effect smooth muscle cells, Pulmonary sarcoidosis, Polycystic
ovary syndrome, Jnruptured intracranial arterial aneurysm, Teratozoospermia, Francisella tularensis
infection, Hormone effect breast cancer cells, Fungal infection, Inflammatory bowel disease, Multiple
sclerosis, Aids related lymphomas
>8195 224086 at
Medicine resistance cancer cells, Pancreatic ductal adenocarcinoma, Medulloblastomas in children,
Idiopathic pulmonary fibrosis, Prechondrocyte differentiation, Probiotic effect on inflamed colonic
explants, Cardiothoracic surgery effect on diaphragm, Tuberculosis, Hutchinson-Gilford progeria
syndrome, Cryptosporidium infection, Fetal Lung development, Familial hypercholesterolemia and
atherosclerosis, Papillary thyroid cancer, Autosomal dominant monocytopenia, Schizophrenia,
Aldosterone-producing adeonoma, Chronic B-lymphocytic leukemia, Optineurin depletion, Metaphase II
stage oocytes maturation, Intracranial arterial aneurysm
(A)biotic stress effect on eukaryotic cells ISCU myopathy
>5927 241867 at
Medicine Desistance cancer cells, Rhinovirus infection, Idiopathic pulmonary fibrosis, AIDS-related
lymphomas, Acute myeloid leukemia, Placental malaria, HIV-associated neurocognitive impairment,
Cardiothoracic surgery, Multiple sclerosis, Nodular lymphocyte-predominant, Hodgkin Lymphoma, HBV-
associated, ALF, Tibial muscular dystrophy, Colorectal cancer primary cr metastatic Lesions,
Inflammatory bowel disease, Parkinson's disease Erythroid differenziation Prechondrocyte
differentiation, Embryonic development, Fetal lung development, Duchenne, Muscular, Dystrophy,
Pancreatic cancer, Optineurin depletion Sjögren's syndrome Ductal carcinoma Franciseila novicida
infections, Polycystic ovary syndrome, Obesity, House dust mite allergy, Chlamydia pneumonia
infection, Systemic lupus erythematosus, Childhood cerebral adrenoleukodystrophy, Multiple sclerosis,
Asthma, Chronic pediatric immune thrombocytopenia, Intracranial arterial aneurysm, Multiple myeloma,
Aldosterone-producing adeonoma, (A)biotic stress effect on eukaryotic cells
>4475 1559434 at
Medicine resistance cancer cells, Tuberculosis, HBV-associated, ALF, Influenza virus infection,
Inflammatory dilated cardiomyopathy, Aldosteronc-producing adconoma, Cushing's Syndromc, (A)biotic
stress effect on eukaryotic cells
>6860 237187 at
Medulloblastoma, Chronic lymphocytic leukemia, Prostate cancer, Pompe disease, Intracranial arterial
aneurysm, Hereditary gingival fibromatosis, Hereditary gingival fibromatosis, Medicine resistance
cancer cells, Hepatitis C Virus infection
>5927 1558889 at
Metaphase II stage oocytes maturation, Influenza infection, Melanoma progression, Systemic lupus
erythematosus and arthritides, Tuberculosis, Alzheimer's disease, Bronchopulmonary dysplasia, Muscles
response exercise and age, Parkinson's disease, Embryonic development, Respiratory syncytial virus
infection, Erythroid differentiation, Heart failure, (A)biotic stress effect on eukaryotic cells,
Heart failure, Early onset colorectal cancer, Chlamydia pneumonia infection, Medicine resistance
cancer cells, Newly ciagnosed and chronic pediatric immune thromb ocytopenia, Aldosterone-producing
adeonoma, Active tuberculosis
>4064 1569416 at
Metaphase II stage oocytes matured, Pancreatic ductal adenocarcinoma, Notch-on/off effects on T-
lymphoblastic leukemia cells, Acute myeloid leukemia, Ulcerative colitis, Melanoma progression,
Tuberculosis, Inflammation vascular epithelial cells, Influenza virus infection, Prostate cancer,
HIV-associated neurocognitive disorders, Francisella tularensis infections, Chronic pediatric immune
thrombocytopenia, Inflammatory dilated cardiomyopathy, Intracranial arterial aneurysm, Childhood
cerebral adrenoleukodystrophy, (A)biotic stress effect on eukaryotic cells, Medicine resistance
cancer cells,
>4865 242495 at
Metaphase TT stage oocytes matured, Early and advanced idiopathic pulmenary fibrosis, Multiple
sclerosis, Placental malaria, Colorectal cancer tumors, Endometriosis, HBV-associated, ALF,
Rhinovirus infection, Embryonic development, Aspergillus infection, Tolerant renal transplant
recipients, Familial hypercholesterolemia and atherosclerosis, Muscle functioning and aging,
Infantile-onset, Pompe disease, Polyomavirus infection, House dust mite allergy, Synchronous and
metachronous liver metastases, EPC response to exercise, Limb girdle muscular dystrophy 2, Obesity,
Pancreatic, Tumor, Neural tube defect, Optineurin depletion, (A)biotic stress effect on eukaryotic
cells Medicine resistance cancer cells,
>8823 220874 at
Multiple myeloma, Chronic B-lymphocytic leukemia, Hepatitis C virus infection, Colorectal cancer,
Teratozoospermia, Medicine resistance cancel cells, COPD lymphoma, Dermatomyositis, Hereditary
gingival fibromatosis, Liver cancer metastasis, Staphylococcus aureus infection, Alcoholic hepatitis
Primary immunodeficiency syndrome Retinoblastomas Asthma, House dust mite allergy, Candida albicans
infection, Hyperlipidemia, Optineurin depletion, Esophagitis, Adenoid cystic carcinoma, HIV
infection, Breast cancer, Toxcplasma gondii infection, Influenza infection, Glioblastoma, Melanoma,
Burkitt lymphoma, Multiple sclerosis, HIV-associated neurocognitive disorders, Psoriasis, Tibial
muscular dystrophy, Hepatitis B virus, (ALF), Acute myeloid leukemia, Presymptomatic and symptomatic
malaria
>4331 238812 at
Multiple sclerosis, Chronic B-lymphocytic leukemia, Plasmodium infection, Acute myeloid leukemia,
Multiple sclerosis, Ovarian endometriosis, Tibial muscular dystrophy, Erythroid differentiation,
Prostate cancer, Endometrium and menstrual cycle, Inflammatory bowel disease, Sjögren's syndrome,
Multistep pancreatic carcinogenesis, Hepatocellular carcinoma, Active tuberculosis, (A)biotic stress
effect on eukaryotic cells, Papillary thyroid cancer, Fetal and adalt reticulocytes, Aldosterone-
producing adeonoma, Muscle function and aging - female, Medicine resistance cancer cells, Asthma
>8195 238301 at
Muscle aging, T-cell acute lymphoblastic leukemia, Medicine resistance cancer cells, Cellular
response to compounds, Teratozoospermia, Chlamydia pneumonia infection, Polycystic ovary syndrome,
Optineurin depletion, Pancreatic, Tumor, Intracranial arterial aneurysm, Preeclampsia, Glioblastoma,
COPD, HPV16 infection, Cushing's syndrome, Breast cancer, Influenza virus infection, Aspergillus
infection, Inflammatory bowel disease, Preeclampsia, Prechondrocyte differentiation, Embryonic
development, Ovarian cancer, Leukemia, Stress effect on skeletal muscle, Prostate cancer, Multiple
sclerosis, Psoriasis, Staphylccoccus aureus infection, Hutchinson-Gilford progeria syndrome, Notch-
on/off effects on T-lymphoblastic leukemia cells, Pancreatic ductal adenocarcinoma, Parkinson's
disease, Rotavirus infection
>4331 242256 x at
Muscle function and aging, Parkinson's disease, Early and advanced idicpathic pulmonary fibrosis,
Williams-Beuren Syndrome, Multiple sclerosis, Alzheimer's disease, Glioblastoma, Tuberculosis,
Plasmodium infection, Fetal lung development, Cryptosporidium infection, Influenza virus infection,
Embrionic development, Aspergillus intection, Prostate cancer ISCU myopathy, Papillary thyroid
cancer, Chlamydia pneumonia infection, Obesity, Polycistic ovary syndrome, Vulvar intraepithelial
neoplasia, Quiescent systemic lupus erythematosus, Synchronous and metachronous liver metastases,
Hereditary gingival fibromatosis, EPC response to exercise, Aldosterone-producing adeonoma, Pituitary
adenoma predisposition, Prechondrocytes, (A)biotic stress effect on eukaryotic cells, Medicine
resistance cancer cells,
>7461 216767 at
Muscles and weight loss, RSV infection, Idiopathic pulmonary fibrosis, Presymptomatic and symptomatic
malaria, Tuberous sclerosis ccmplex hamartomas, Systemic lupus erythematosus and arthritides,
Neisseria meningitidis infection, Rheumatoid arthritis, Adenocarcinoma, Radioresistant tumor
response, Sindbis virus infection, Multistep pancreatic carcinogenesis, Essential thrombocythemia, T-
cell prolymphocytic leukemia, B-cell chronic lymphocytic leukemia, Embryonic development,
Preeclampsia, Duchenne, Muscular, Dystrophy, Myelodysplastic syndrome, Influenza virus infection,
Huntington's disease, Familial hypercholesterolemia and atherosclerosis, Liver transplantation
tolerance, Metaphase II stage oocytes maturation Staphylococcus aureus infection Prostate cancer
Nephrosclerosis Multiple sclerosis Pulmonary type II cell differentiation Chronic pediatric immune
thrombocytopenia Chlamydia pneumonia infection Polycystic ovary syndrome Retinoblastoma Diamond-
Blackfan anaemia Papillary thyroid cancer Melanoma metastasis Obesity Carious pulpal tissue Asthma
Nasopharyngeal carcinoma Aldosterone-producing adeonoma Porphyromonas gingivalis infection
Intracranial arterial aneurysm (A)biotic stress effect on eukaryotic cells Medicine resistance cancer
cells Dermatomyositis,
>2836 1562642 at
Nephroblastoma Obese HBV virus infection Intracranial arterial aneurysm Medicine resistance cancer
cells Optineurin depletion Colorectal cancer Fanccreatic Tumor HPV16 infection Plasmodium falciparum
infection Prostatc cancer Colcrectal cancer metastatic lesions
>6113 1566425 at
Oocytes metaphase Medicine resistance cancer cells Granulocyte
colony-stimulating factor mobilized leukocytes Parkinson's disease
>250 1569603 at Pancreatic tumor Embryonic development Adenoid cystic carcinoma Ovarian cancer Colon
cancer Invasive ductal and lobular breast carcinomas Influenza viris infection Hormone effect on
postmenopausal endometrium Familial hypercholesterolemia and atherosclerosis Francisella novicida
infection Medicine resistance cancer cells (A)biotic stress effect on eukaryotic cells Metaphase II
stage oocytes maturation Chlamydia pneumonia infection Early onset colorectal cancer Sonic hedgehog
homolog
>6009 1562553 at
Papillary thyroid cancer (A)biotic stress effect on eukaryotic cells Medicine resistance cancer cells
Optineurin depletion PDCCE tumor Pancreatic tumor Pituitary adenoma predisposition Acute myeloid
leukemia Staphylococcus aureus infection Cushing's syndrome Prostahe cancer HPV16 virus infection
Heart failure Aspergillus infection Influenza virus infection Ovarian cancer Prechondrocyte
differentiation Plasmodium infection Hepatitis B virus infection (ALF) Embryonic development
>8407 241468 at
Papillary thyroid cancer Breast cancer Multiple Sclerosis Plasmodium falciparum infection
Prechondrocyte differentiation Lung carcinoma Influenza virus infections Herpesvirus infection
Prostate cancer Chronic B-lymphocytic leukemia Medicine resistance cancer cells Intracranial arterial
aneurysm Liver transplantation tolerance Simian immune deficiency virus infection Multiple Sclerosis
Optineurin depletion
>676 233235 x at
Polycystic ovary syndrome Obesity (A)biotic stress effect on eukaryotic cells Stage I endometrial
cancer Skeletal muscle and age Endometriosis Ovarian endometriosis Prostate cancer Embryonic
development Preeclampsia Prechondrocyte differentiation Duchenne Muscular Dystrophy Heart failure
Adenoid cystic carcinoma Chronic B-lymphocytic leukemia Liver transplantation
tolerance Metaphase II stage oocytes maturation Autosomal dominant monocytopenia
Teratozoospermia Intracranial arterial aneurysm Muscle function and aging - male Medicine resistance
cancer cells Chronic pediatric immune thrombocytopenia
>9005 234654 at Polyomavirus JCV infection Teratospermia Medicine resistance cancer cells Simian
immune deficiency virus infection Francisella
tularensis infection Prechondrocyte differentiation Multiple Sclerosis Mycobacterium
tuberculosis infection
>9101 1562586 at
Prostate cancer Intracranial arterial aneurysm Influenza virus infection Medicine resistance cancer
cells Plasmodium falciparum infection Rheumatoid arthritis Tendinopathy Pancreatic ductal
adenocarcinoma
>8198 1560230 at
Pulmonary sarcoidosis (A)biotic stress effect on eukaryotic cells Psoriasis Lassa fever Tendinopathy
Jlcerative colitis Melanoma progression Ovarian endometriosis Cervical cancer tumors Papillary
thyroid carcinoma Pituitary genadotrope tumors Tibial muscular dystrophy Plasmodium infection
Parkinson's disease Embryonic development Ovarian cancer Prechondrocyte differentiation Endometrium
and menstrual cycle Adenoid cystic carcinoma Influenza virus infection Tibolone hormone effect
on postmenopausal endometrium Pancreatic cancer Small cell lung cancers Sjögren's syndrome
Aspergillus infectior Basal-like breast cancer tumors Hepatitis C virus infection Francisella
novicida infections Obesity HIV infection Papillary thyroid cancer Chronic B-lymphocytic leckemia
Polycystic ovary syndrome Chronic pediatric immune thrombocytopenia House dust mite allergy Medicine
resistance cancer cells Autosomal dominant monocytopenia
>4064 1561737 atSchizophrenia Chronic B-lymphocytic leukemia Medicine resistance cancer cells Obesity
Acute myeloid leukemia Chlamydia
pneumonia infection Acute myccardial infarction Pulmonary sarcoidosis Hormone effect cancer cells
Immature dendritic cell response to hypoxia Chromophobe renal cell carcinoma Hereditary gingival
fibromatosis Metaphase II stage oocytes maturation HIV and SIV infection Aspergillus infection
Sjögren's syndrome Embryonic development Prechondrocyte differentiation HIV-associated
neurocognitive disorders Psoriasis Inflammation
vascular epithelial cells Staphylococcus aureus infection
Hodgkin and non-Hodgkin lymphoma Notch-on/off effects on
T-lymphoblastic Leukemia cells>6914 1564984 at Sepsis Obesity Medicine resistance cancer cells
Jnruptured intracranial arterial aneurysm Pituitary adenoma predisposition Acute leukemia Vulvar
intraepithelial neoplasia Interleukin therapeutic Polycystic ovary syndrome House dust mite allergy
Multiple Sclerosis Optineurin depletion Prostate cancer linoleic acid isomers exposure response
Influenza infection Fungi infection Pancreatic cancer IFNß-1a treatment multiple sclerosis patients
Idiopathic pulmonary fibrosis
>8792 242795 at
Sepsis Optineurin depletion Medicine resistance cancer cells Teratozoospermia Pancreatic cancer
Polyomavirus JCV resistance Prostate cancer progression Cystic fibrosis COPD Cryptosporidium
infection Type 2 diabetes Inflammation vascular epithelial cells Tibial muscular dystrophy
Staphylococcus aureus infection
>7129 1555664 at
Sepsis Sickle cell disease Metaphase II stage oocytes maturation Intracranial arterial aneurysm
Hormone effect on cancer cells Dystocic myonetrium Medicine resistance cancer cells Optineurin
depletion Schizophrenia Aids Colorectal cancer Pituitary adenoma Familial hypercholesterolemia and
atherosclerosis linoleic acid isomers effect on eukaryotic cells Hepatitis C virus infection
Influenza virus infection Prostate cancer Preeclampsia Embryonic development Ovarian cancer Notch-
on/off effects on T-lymphoblastic leukemia cell lines
>8195 215928 at
Systemic lupus erythematosus Colon cancer Pompe disease Sepsis Medicine resistance cancer cells
Asthma Cellular responds do compounds Limb girdle muscular dystrophy Down syndrome Aldosterone-
producing adeonoma Chlamydia infection Dystocic myometrium Vulvar intraepithelial neoplasia COPD
Pulmonary sarcoidosis Ewing family tumor and neuroblastoma comparison In
flammatory dilated cardiomyopathy Obesity SIV virus infection Smoking effect on B lymphocytes in
females Nephroblastoma Idiopathic yelofibrosis Kaposi sarcoma Toxoplasma gondii infection Rheumatoid
arthritis Bipolar disorder Prechondrocyte differentiation Cryptosporidium infection Tibial muscular
dystrophy Renal clear cell carcinoma Hungington's disease Adenocarcinoma
Ewing Sarcoma Malaria CD4+ T cell differentiation Exercise effect on whitc blood cells
Celiac disease Notch-on/off effects on T-lymphoblastic leukemia cells SCID disease Essential
thrombocythemia Pituitary adenoma subtypes
>9342 242924 at
Teratospermia myeloma Prechondrocyte differentation Alzheimer's disease
>5170 1561642 at
Teratozoospermia Pituitary gonadotrope tumors Tendinopathy Tuberculosis Preeclampsia Prostaue cancer
Multiple sclerosis Cryptosporidium infection RSV infection Embryonic development Ovarian cancer
Sjögren's syndrome Francisella tularensis infections Heart failure HIV infection Obesity (A)biotic
stress effect on eukaryotic cells Medicine resistance cancer cells,
>8960 1559724 at
Therapeutic or toxin effects on cell lines Chronic B-lymphocytic leukemia Autosomal dominant
monocytopenia Vulvar intraepithelial neoplasia Medicine resistance cancer cells Optinetrin depletion
Asthma Francisella tularensis infections Basal-like breast cancer tumors Hepatitis C Virus infection
Ovarian cancer HIV-associated neurocognitive disorders Psioriasis Leishmania infection Endotoxin
effect immune cells Notch-on/
off effects on T-Lymphoblastic leukemia cells

List 2

>639_205481_at
adenosine, dyphylline, aspirin/butalbital/caffeine, acetaminophen/caffeine/dihydrocodeine,
acetaminophen/aspirin/caffeine, caffeine/ergotamine, aspirin/caffeine/propoxyphene,
aspirin/butalbital/caffeine/codeine, aspirin/caffeine/dihydrocodeine, caffeine/ethanol,
acetaminophen/butalbital/caffeine, aminophylline, aspirin/caffeine/orphenadrine, clofarabine,
acetaminophen/butalbital/caffeine/codeine, theophylline, pentoxifylline, caffeine, theobromine,
tecadenoson, acetaminophen/caffeine/chlorpeniramine/hydrocodone/phenylephrine
>676_202707_at
pyrazofurin, gimeracil/oxonic acid/tegafur, oxonic acid
>6131_210005_at
bevacizumab/pemetrexed, gemcitabine/pemetrexeby pemetrexed
>6431_206482_at
vandetanib
>6647_206070_at
SF 1126, PX-866, PI3Kg inhibitor, dactolisib, pictilisib, buparlisib, XL147, PQR309, RP6530
>6661_237469_at
novobiocin, etoposide, teniposide, CPI-0004Na, becatecarin, elsamitrucin, AQ4N, elomotecan,
tafluposide, fleroxacin, cyclophosphamide/epirubicin/5-fluorouracil, cytarabine/daunorubicin,
finafloxacin, hydrocortisone/mitoxantrone, mitoxantrone/prednisone,
cladribine/cytarabine/daunorubicin, cytarabine/daunorubicin/tretinoin, cytarabine/idarubicin,
idarubicin/mitoxantrone/tretinoin, arsenic trioxide/daunorubicin/tretinoin, cytarabine/etoposide,
cytarabine/etoposide/mitoxantrone, cytarabine/mitoxantrone, daunorubicin/tretinoin,
idarubicin/tretinoin, bortezomib/dexamethasone/doxorubicin, cisplatin/doxorubicin/methotrexate,
cyclophosphamide/epirubicin, cyclophosphamide/etoposide,
cisplatin/doxorubicin/ifosfamide/methotrexate, cisplatin/doxorubicin,
cyclophosphamide/docetaxel/epirubicin/5-fluorouracil/trastuzumab,
clofarabine/cyclophosphamide/etoposide, cyclophosphamide/epirubicin/vincristine,
bortezomib/doxorubicin, carboplatin/doxorubicin, cladribine/cytarabine/filgrastim/mitoxantrone,
epirubicin/5-fluorouracil/oxaliplatin, dacarbazine/doxorubicin, cytarabine/filgrastim/fludarabine
phosphate/idarubicin, capecitabine/epirubicin/oxaliplatin, doxorubicin/ifosfamide,
cladribine/cytarabine/filgrastim/idarubicin, doxorubicin/streptozocin,
dacarbazine/doxorubicin/ifosfamide, dexamethasone/fludarabine phosphate/mitoxantrone,
cyclophosphamide/etoposide/prednisone/rituximab/
vincristine, cyclophosphamide/fludarabine phosphate/mitoxantrone/rituximab, fludarabine
phosphate/mitoxantrone/rituximab, cyclophosphamide/mitoxantrone/prednisone/vincristine,
cyclophosphamide/etoposide/prednisone/vincristine, dexamethasone/fludarabine
phosphate/mitoxantrone/rituximab, chlorambucil/mitoxantrone/prednisone, fludarabine
phosphate/mitoxantrone, cyclophosphamide/fludarabine phosphate/mitoxantrone,
alemtuzumab/cyclophosphamide/fludarabine phosphate/mitoxantrone,
cyclophosphamide/mitoxantrone/prednisone/rituximab/vincristine,
cladribine/cytarabine/filgrastim/idarubicin/plerixafor, doxorubicin/tretinoin,
cytarabine/doxorubicin, chlorambucil/mitoxantrone/prednisone/rituximab, docetaxel/epirubicin,
doxorubicin/paclitaxel, epirubicin/paclitaxel, arsenic trioxide/idarubicin/tretinoin,
clofarabine/filgrastim/idarubicin, clofarabine/cytarabine/filgrastim/idarubicin,
cyclophosphamide/epirubicin/5-fluorouracil/vinorelbine, cyclophosphamide/epirubicin/5-
fluorouracil/paclitaxel/trastuzumab, cytarabine/idarubicin/sorafenib, cyclophosphamide/epirubicin/5-
fluorouracil/paclitaxel, cyclophosphamide/docetaxel/epirubicin/5-fluorouracil, mitoxantrone,
amsacrine, norfloxacin, dexrazoxane, tirapazamine, TAS-103, gatifloxacin, valrubicin, gemifloxacin,
nemorubicin, nalidixic acid, levofloxacin, epirubicin, doxorubicin, idarubicin, daunorubicin,
nodophyllotoxin, cis-sparfloxacin
>6713_229711_s_at
CGM097, DS-3032b, HDM201, RO5503781, RO6839921
>6811_207822_at
pazopanib, lenvatinib, nintedanib, regorafenib, bortezomib/sorafenib,
dexamethasone/lenalicomide/sorafenib, GSK3052230, lucitanib, cytarabine/idarubicin/sorafenib, 5-
azacytidine/sorafenib, decitabine/sorafenib, cebio 1347, ARQ 087, sulfatinib, everolimus/sorafenib,
sorafenib
>6811_224029_x_at
riluzole, zonisamide
>6859_244616_x_at
CGM097, DS-3032b, HDM201, RO5503781, RO6839921
>7567_210224_at
anti-thymocyte globulin (rabbit)
>7567_203672_x_at
olsalazine
>7984_1555572_at
methazolamide, hydrochlorothiazide, acetazolamide, trichloromethiazide, chlorothiazide,
chlorthalidone, benzthiazide, sulfacetamide, topiramate
>7384_1570200_at
cyclophosphamide/epirubicin/5-fluorouracil, cyclophosphamide/epirubicin,
cyclophosphamide/docetaxel/epirubicin/5-fluorouracil/trastuzumab,
cyclophosphamide/epirubicin/vincristine, epirubicin/5-fluorouracil/oxaliplatin,
capecitabine/epirubicin/oxaliplatin, docetaxel/epirubicin, epirubicin/paclitaxel,
cyclophosphamide/epirubicin/5-fluorouracil/vinorelbine, cyclophosphamide/epirubicin/5-
fluorouracil/paclitaxel/trastuzumab, cyclophosphamide/epirubicin/5-fluorouracil/paclitaxel,
cyclophosphamide/docetaxel/epirubicin/5-fluorouracil, epirubicin
>8015_208306_x_at
apolizumab
>8015_244689_at
choline fenofibrate, aleglitazar, fenofibric acid/rosuvastatin, atorvastatin/choline fenofibrate,
choline fenofibrate/simvastatin, ezetimibe/fenofibrate, fenofibrate/simvastatin, NS-22C,
tesaglitazar, bezafibrate, clofibrate, fenofibrate, docosahexaenoic acid, gemfibrozil
>8015_206792_x_at
dyphylline, nitroglycerin, caffeine/ergotamine, arofylline, tetomilast, L 869298, ibudilast,
aminophylline, anagrelide, cilomilast, milrinone, dipyridamole, L-326, 141, ketotifen, roflumilast,
tolbutamide, theophylline, pentoxifylline, caffeine
>8015_1552343_s_at
dyphylline, nitroglycerin, aminophylline, anagrelide, milrinone, dipyridamole, ketotefen,
tolbutamide, theophylline, pentoxifylline
>8015_1569748_at
pkc-zeta inhibitor, ingenol mebutate
>8015_216439_at
vemurafenib, osimertinib, cobimetinib/vemurafenib
>5925_201695_s_at
forodesine, PD 141955, purine nucleoside phosphorylase inhibitor
>5927_204920_at
N-carbamyl-L-glutamate
>5927_215179_x_at
aflibercept, aflibercept/irinotecan
>8195_207516_at
ABT-089, isoflurane, mecamylamine, succinylcholine, rocuronium, doxacurium, mivacurium, pipecuronium,
rapacuronium, metocurine, atracurium, cisatracurium, acetylcholine, nicotine, D-tubocurarine,
arecoline, ibogaine, enflurane, pancuronium, vecuronium
>8306_211265_at
misoprostol, prostaglandin E2, prostaglandin E1, diclofenac/misoprostol, bimatoprost
>8306_1569938_at
suramin
>8319_207554_x_at
S-18886, ridogrel, torsemide
>8319_1552648_a_at
trm-1
>19_221170_at
tesmilitene, triprolidine, buclizine
>8889_244677_at
avibactam
>8998_208828_at
bortezomib/cladribine/rituximab, gemcitabine/paclitaxel, cladribine/cytarabine/daunorubicin,
gemcitabine/oxaliplatin, gemcitabine/vinorelbine, docetaxel/gemcitabine,
docetaxel/gemcitabine/vincristine, cladribine/cytarabine/filgrastim, capecitabine/gemcitabine,
gemcitabine/oxaliplatin/paclitaxel, cladribine/cytarabine/filgrastim/mitoxantrone,
cladribine/rituximab, capecitabine/docetaxel/gemcitabine,
cladribine/cytarabene/filgrastim/idarubicin, bevacizumab/gemcitabine,
gemcitabine/oxaliplatin/rituximab, L-asparaginase/gemcitabine/oxaliplatin,
cladribine/cytarabine/filgrastim/idarubicin/plerixator, gemcitabine/irinotecan, 5-
fluorouracil/gemcitabine, cladribine/peginterferon alfa-2a, gemcitabine/pemetrexed,
cladribine/cytarabine/decitabine, cyclophosphamide/gemcitabine/prednisclone/rituximab/vincristine,
gemcitabine, cladribine
>9002_216288_at
zeneca ZD 3523, montelukast, zafirlukast, pranlukast
>9035_203989_x_at
chrysalin, vorapaxar, argatroban, bivalirudin
>921_211286_x_at
sargramostim, mavrilimumab, EPO/sargramostim, rituximab/sargramostim
>921_213475_s_at
efalizumab
>921_1561080_at
arsenic trioxide/tretinoin, arsenic trioxide/caunorubicin/tretinoin, arsenic trioxide/gemtuzumab
Ozogamicin/tretinoin, arsenic trioxide/idarubicin/tretinoin, ethaselen, arsenic trioxide
>9365_213631_x_at
teriflunomide, leflunomide
>1711_215365_at
amlodipine/hydrochlorothiazide/valsartan, amlodipine/telmisartan, aliskiren/amlodipine,
aliskiren/amlodipine/hydrochlorothiazide, nilvadipine, amlodipine/valsartan, amlodipine/perindopril,
amlodipine/benazepril, verapamil, mibefradil, enalapril/felodipine, amlodipine/atorvastatin,
nisoldipine, isradipine, felodipine, nimodipine, nitrendipine, amlodipine, nicardipine, nifedipine,
trandolapril/veapamil
>1723_210389_x_at
epothilone B, colchicine/probenecid, larotaxel, eribulin, davunetide, vintafolide, milataxel,
cevipabulin, gemcitabine/paclitaxel, docetaxel/prednisone, capecitabine/docetaxel,
paclitaxol/trastuzumab, cyclophosphamide/predrisone/vincristine, docetaxol/hydrocortisone,
cyclophosphamide/docetaxel, gemcitabine/vinorelbine,
cyclophosphamide/daunorubicin/imatinib/prednisone/vincristine,
cyclophosphamide/topotecan/vincristine, docetaxel/gemcitabine, docetaxel/gemcitabine/vincristine,
irinetecan/vincristine, irinotecan/temozolomice/vincristine, bevacizumab/paclitaxel,
cyclophosphamide/docetaxel/epirubicin/5-fluorouracil/trastuzumab, docetaxel/trastuzumab,
trastuzumab/vinorelbine, gemcitabine/oxaliplatin/paclitaxel, cyclophosphamide/epirubicin/vincristine,
docetaxel/irinotecan, docetaxel/5-fluorouracil/oxaliplatin, capecitabine/docetaxel/gemcitabine, L-
asparaginase/prednisone/vincristine, cyclophosphamide/etoposide/prednisone/rituximab/vincristine,
cyclophosphamide/vinorelbine, cyclophosphamide/mitoxantrone/prednisone/vincristine,
cyclophosphamide/etoposide/prednisone/vincristine, cyclophosphamide/prednisone/rituximab/vincristine,
cyclophosphamide/mitoxantrone/prednisone/rituximab/vincristine, plinabulin, docetaxel/epirubicin,
docetaxel/paclitaxel, epirubicin/paclitaxel, bevacizumab/paclitaxel/topotecan, paclitaxel/topotecan,
bevacizumab/docetaxel, cyclophosphamide/prednisolone/vincristine,
cyclophosphamide/prednisolone/rituximab/vincristine, cyclophosphamide/epirubicin/5-
fluorouracil/vinorelbine, cyclophosphamide/epirubicin/5-fluorouracil/paclitaxel,/trastuzumab,
cyclophosphamide/epirubicin/5-fluorouracil/paclitaxel, cyclophosphamide/docetaxel/epirubicin/5-
fluorouracil, cyclophosphamide/docetaxel/trastuzumab,
cyclophosphamide/gemcitabine/prednisolone/rituximab/vincristine, docetaxel, vinflunine, vinorelbine,
vincristine, vinblastine, paclitaxel,
podophyllotoxin, colchicine
>1792_211277_x_at
bapineuzumab, florbetapir F18, florbetaben F
>2049_202015_x_at
nitroxoline, beloranib, XMT-1107, PPI-2458
>2080_207536_s_at
urelumab
>130_205628_at
cytarabine/fludarabine phosphate, cyclophosphamide/fludarabine phosphate/rituximab,
cytarabine/filgrastim/fludarabine phosphate, fludarabine phosphate/rituximab,
dexamethasone/fludarabine phosphate/mitoxantrone, alemtuzumab/cyclophosphamide/fludarabine
phosphate/rituximab, alemtuzumab/fludarabine phosphate, cytarabine/fludarabine
phosphate/oxaliplatin/rituximab, cyclophosphamide/fludarabine phosphate/mitoxantrone/rituximab,
fludarabine phosphate/mitoxantrone/rituximab, dexamethasone/fludarabine
phosphate/mitoxantrone/rituximab, fludarabine phosphate/mitoxantrone, cyclophosphamide/fludarabine
phosphate/mitoxantrone, alemtuzumab/cyclophosphamide/fludarabine phosphate/mitoxantrone,
chlorambucil/fludarabine phosphate, fludarabine phosphate
>2365_1559206_at
tarenflurbil
>2379_210636_at
treprostinil, icosapent, GW501516, bezafibrate
>2383_214601_at
5,6,7,8-tetrahydrobiopterin
>2497_230808_at
lonafarnib
>2747_1559420_x_at
amlodipine/hydrochlorothiazide/valsartan, amlodipine/telmisartan, aliskiren/amlodipine,
aliskiren/amlodipine/hydrochlorothiazide, nilvadipine, amlodipine/valsartan, amlodipine/perindopril,
amlodipine/benazepril, verapamil, mibefrafil, enalapril/felodipine, amlodipine/atorvastatin,
nisoldipine, isradipine, felodipine, nimodipine, nitrendipine, amlodipine, nicardipine, nifedipine,
trandolapril/verapamil
>2747_1559419_at
amlodipine/hydrochlorothiazide/valsartan, amlodipineite/telmisartan, aliskiren/amlodipine,
aliskiren/amlodipine/hydrochlorothiazide, nilvadipine, amlodipine/valsartan, amlodipine/perindopril,
amlodipine/benazepril, verapamil, mibefradil, enalapri1/felodipine, amlodipine/atorvastatin,
nisoldipine, isradipine, felodipine, nimodipine, nitrendipine, amlodipine, nicardipine, nifedipine,
trandolapril/verapamil
>3395_220791_x_at
riluzole, zonisamide
>3609_215404_x_at
pazopanib, lenvatinib, nintedanib, regorafenib, bortezomib/sorafenib,
dexamethasone/lenalicomide/sorafenib, GSK3052230, lucitanib, cytarabine/idarubicin/sorafenib, 5-
azacytidine/sorafenib, decitabine/sorafenib, cebio 1347, ARQ 087, sulfatinib, everolimus/sorafenib,
sorafenib
>3626_202532_s_at
pyrimethamine, trimethoprim, iclaprim, proguanil, methotrexate/ofazumumab,
methotrexate/sirolimus/tacrolimus, pralatrexate, abatacept/methotrexate, infliximab/methotrexate,
methotrexate/rituximab, golimumab/methotrexate, cisplatin/doxorubicin/methotrexate,
cytarabine/methotrexate, cisplatin/doxorubicib/ifosfamide/methotrexate,
methotremate/rituximab/temozolomide, ifosfamibe/methotrexate, bevacizumab/pemetrexed,
cytarabine/dexamethasone/methotrexate, leucovorin/methotrexate, cyclophosphamide/methotrexate,
adalimumab/methotrexate, gemcitabine/pemetrexed, epinephrine/methotrexate, dihydrofolate reductase
inhibitor, ABTL0812, methotrexate, atovaquone/proguanil, sulfisoxazole, triamterene, folic acid,
trimetrexate, pemetrexed, talctrexin
>3953_210528_at
anti-thymocyte globulin (rabbit)
>3953_210771_at
choline fenofibrate, aleglitazar, fenofibric acid/rosuvastatin, atorvastatin/choline fenofibrate,
choline fenofibrate/simvastatin, ezetimibe/ferofibrate, fenofibrate/simvastatin, NS-22C,
tesaglitazar, bezafibrate, cicfibrate, fenofibrate, docosahexaenoic acid, gemfibrozil
>4331_1557197_a_at
GR-MD-02
>4331_215709_at
cytarabine/fludarabire phosphate, cyclophosphamide/fludarabine phosphate/rituximab,
cytarabine/filgrastim/fludarabine phosphate, fludarabine phosphate/rituximab,
dexamethosone/fludarabine phosphate/mitoxantrone, alemtuzumab/cyclophosphamide/fludarabine
phosphate/rituximab, alemtuzumab/fludarabine phosphate, cytarabine/fludarabine
phosphate/oxaliplatin/rituximab, cyclophosphamide/fludarabine phosphate/mitoxantrone/rituximab,
fludarabine phosdhate/mitoxantrone/rituximab, dexamethasone/fludarabine
phosphate/mitoxantrone/rituximab, fludaradine phosphate/mitoxantrone, cyclophosphamide/fludarabine
phosphate/mitoxantrone, alemtuzumab/cyclophosphamide/fludarabine phosphate/mitoxantrone,
chlorambucil/fludarabine phosphate, fludarabine phosphate
>4426_1558199_at
ocriplasmin, L19-IL2 monoclonal antibody-cytokine fusion protein
>4484_205998_x_at
cobicistat, cobicistat/elvitegravir/emtricitabine/tenofovir disoproxil, atazanavir/cobicistat,
cobicistat/darunavir, ketoconazole
>4484_231775_at
trm-1
>4621_216261_at
abciximab, TP 9201, cilengitide, tirofiban
>379_201404_x_at
carfilzomib, bortezomib/cladribine/rituximab, bortezomib/sorafenib, bortezomib/paclitaxel,
bortezomib/dexamethasone, bortezomib/dexamethasone/doxorubicin,
bortezomib/dexamethasone/lenalidomide, bortezomib/dexamethasone/thalidomide,
carfilzomib/dexamethasone/lenalidomide, bortezomib/prednisone, bortezomib/doxorubicin,
bortezomib/vorinostat, bortezomib/thalidomide, bortezomib/rituximab,
bortezomib/dexamethasone/rituximab, carfilzomib/dexamethasone/rituximab, bortezomib
>379_210917_at
dasatinib
>5212_1569554_at
17-alpha-ethinylestradiol, fulvestrant, beta-estradiol, estradiol 17beta-cypionate, estrone,
estradiol valerate, CHF-4227, bazedoxifene, estradiol valerate/tesfosterone enanthate, TAS-108,
ethinyl estradiol/ethynodiol diacetate, estrabiol acetate, esterified estrogens, estradiol
cypionate/medroxyprogesterone acetate, estradiol/norethindrone acetate, estradiol
cypionate/testosterore cypionate, synthetic conjugated estrogens, 3, CC8490, MITO-4509, megestrol
acetate/tamoxifen, sulindac/tamoxifen, sulindac/toremifene, raloxifene/sulindac, goserelin/tamoxifen,
raloxifene/teriparatide, desogestrel/ethinyl estradiol, drospirenone/ethinyl estradiol, premarin,
ethinyl estradiol/norelgestromin, ethinyl estradiol/norethindrone, ethinyl estradiol/levonorgestrel,
ethinyl estradiol/norgestrel, ethinyl estradiol/norgestimate, conjugated estrogen/medroxyprogesterone
acetate, diethylstilbestrol, ospemifene, toremifene, tamoxifen, prinaberel, raloxifene, arzoxifene,
clomiphene, estramustine phosphate, diethylstilbestrol diphosphate, estropipate, genistein,
trilostane
>5212_1554710_at
tedisamil
>9457_201746_at
CGM097, kevetrin
>9460_211590_x_at
S-18886, ridogrel, torsemide
>9501_211164_at
anti-EphA3 monoclonal antibody KB004
>504_204713_s_at
drotrecogin alfa, antithrombin alfa
>9831_221152_at
collagenase clostridium histolyticum
>559_210005_at
bevacizumab/pemetrexed, gemcitabine/pemetrexec, pemetrexed
>559_244689_at
choline fenofibrate, aleglitazar, fenofibric acid/rosuvastatin, atorvastatin/choline fenofibrate,
choline fenofibrate/simvastatin, ezetimibe/fenofibrate, fenofibrate/simvastatin, NS-22C,
tesaglitazar, bezafibrate, clofibrate, fenofibrate, docosahexaenoic acid, gemfibrozil
>639_205481_at
adenosine, dyphylline, aspirin/butalbital/caffeine, acetaminophen/caffeine/dihydrocodeine,
acetaminophen/aspirin/caffeine, caffeine/ergotamine, aspirin/caffeine/propoxyphene,
aspirin/butalbital/caffeine/codeine, aspirin/caffeine/dihydrocodeine, caffeine/ethanol,
acetaminophen/butalbital/caffeine, aminophylline, aspirin/caffeine/orphenadrine, clotarabine,
acetaminophen/butalbital/caffeine/codeine, theophylline, pentoxifylline, caffeine, theobromine,
tecadenoson, acetaminophen/caffeine/chlorpheniramine/hydrocodone/phenylephrine
>720_216261_at
abciximab, TP 9201, cilengitide, tirofiban
>796_233109_at
collagenase clostridium histolyticum
>796_211286_x_at
sargramostim, mavrilimumab, EPO/sargramostim, rituximab/sargramostim
>796_210364_at
riluzole, zonisamide
>796_1569554_at
17-alpha-ethinylestradiol, fulvestrant, beta-estradiol, estradiol 17beta-cypionate, estrone,
estradiol valerate, CHF-4227, bazedoxifene, estradiol valerate/testosterone enanthate, TAS-108,
ethinyl estradiol/ethynodiol diacetate, estradiol acetate, esterified estrogens, estradiol
cypionate/medroxyprogesterone acetate, estradiol/norethindrone acetate, estradiol
cypionate/testosterone cypionate, synthetic conjugated estrogens, 3, CC8490, MITO-4509, megestrol
acetate/tamoxifen, sulindac/tamoxifen, sulindac/toremifene, raloxifene/sulindac, goserelin/tamoxifen,
raloxifene/teriparatide, desogestrel/ethinyi estradiol, drospirenone/ethinyl estradiol, premarin,
ethinyl estradiol/norelgestromin, ethinyl estradiol/norethindrone, ethinyl estradiol/levonorgestrel,
ethinyl estradiol/norgestrel, ethinyl estradiol/norgestimate, conjugated estrogen/medroxyprogesterone
acetate, diethylstilbestrol, ospemifene, toremifene, tamoxifen, prinaberel, raloxifene, arzoxifene,
clomiphene, estramustine phosphate, diethylstilbestrol diphosphate, estropipate, gendstein,
trilostane
>796_1559206_at
tarenflurbil
>808_214019_st
arsenic trioxide/tretinoin, arsenic trioxide/daunorubicin/tretinoin, arsenic trioxide/gemtuzumab
ozogamicin/tretinoin, arsenic trioxide/idarubicin/tretinoin, arsenic trioxide
>5679_1554710_at
tedisamii
>6249_220791_x_at
riluzole, zonisamide
>5732_203672_x_at
olsalazine
>5736_208306_x_at
apolizumab
>6358_211627_x_at
17-alpha-ethinylestradiol, fulvestrant, beta-estradiol, estradiol 17beta-cypionate, estriol, estrone,
estradiol valerate, estrone sulfate, mestranol, CHF-4227, bazedoxifene, estradiol
valerate/testosterone enanthate, TAS-108, ethynodiol diacetate, ethinyl estradiol/ethynodiol
diacetate, estradiol acetate, esterified estrogens, estradiol cypionate/medroxyprogesterone acetate,
estradiol/norethindrone acetate, estradiol cypionate/testosterone cypionate, synthetic conjugated
estrogens, B, etonogestrel, CC8490, MITO-4509, cyproterone acetate/ethinyl estradiol, ethinyl
estradiol/etonogestrel, chlorctrianisene, megestrol acetate/tamoxifen, sulindac/tamoxifen,
sulindac/toremifene, raloxifene/sulindac, AN-810, goserelin/tamoxifen, raloxifene/teriparatide,
RAD1901, SRN-927, fulvestrant/palbociclib, desogestrel/ethinyl estradiol, drospirenone/ethinyl
estradiol, premarin, ethinyl estradiol/norelgestromin, ethinyl estradiol/norethindrone, ethinyl
estradiol/levonorgestrel, ethinyl estradiol/norgestrel, ethinyl estradiol/norgestimate, conjugated
estrogen/medroxyprogesterone acetate, diethylstilbestrol, ospemifene, toremifene, tamoxifen,
raloxifene, arzoxifene, clomiphene, estramustine phosphate, diethylstilbestrol diphosphate,
estropipate, 4-hydroxytamoxifen, dienestrol, estramustine, medroxyprogesterone acetate, desogestrel,
danazol, trilostane, fluoxymesterone, norgestimate, progesterone
>6366_203084_at
dalantercept
>6619_202015_x_at
nitroxoline, beloranib, XMT-1107, PPI-2458
>6698_216288_at
zeneca ZD 3523, montelukast, zafirlukast, pranlukast
>6713_229711_s_at
CCM097, DS-3032b, HDM201, R05503781, R06839921
>5873_207554_x_at
S-18886, ridogrel, torsemide
>New 33_210917_at
dasatinib
>5969_217425_at
cosyntropin, corticotropin
>7700_238425_at
carfilzomib, bortezomib/cladribine/rituximab, bortezomib/sorafenib, bortezomib/paclitaxel,
bortezomib/dexamethasone, bortezomib/dexamethasone/doxorubicin,
bortezomib/dexamethasone, lenalidomide, bortexomib/dexamethasone/thalidomide,
carfilzomib/dexamethasone/lenalidomide, bortexomib/prednisone, bortezomib/doxorubicin,
bortezomib/vorinostat, bortezomib/thalidomide, bortezomib/rituximab,
bortezomib/dexamethasone/rituximab, carfilzomib/dexamethasone/rituximab, bortezomib
>7992_205386_s_at
CGM097, D5-3032b, HDM201, RO5503781, RO6839921
>8015_205945_at
tocilizumab
>5618_201746_at
CGM097, kevetrin
>8303_215709_at
cytarabine/fludarabine phosphate, cyclophosphamide/fludarabine phosphate/rituximab,
cytarabine/filgrastim/fludarabine phosphate, fludarabine phosphate/rituximab,
dexamethasone/fludarabine phosphate/mitoxantrone, alemtuzumab/cyclophosphamide/fludarabine
phosphate/rituximab, alemtuzumab/fludarabine phosphate, cytarabine/fludarabine
phosphate/oxaliplatin/rituximab, cyclophosphamide/fludarabine phosphate/mitoxantrone/rituximab,
fludarabine phosphate/mitoxantrone/rituximab, dexamethasone/fludarabine
phosphate/mitoxantrore/rituximab, fludarabine phosphate/mitoxantrone, cyclophosphamide/fludarabine
phosphate/mitoxantrone, alemtuzumab/cyclophosphamide/fludarabine phosphate/mitoxantrone,
chlorambucil/fludarabine phosphate, fludarabine phosphate
>8386_202812_at
miglito1, acarbose
>8766_207822_at
pazopanib, lenvatinib, nintedanib, regorafenib, bortezomib/sorafenib,
dexamethasone/lenalidomide/sorafenib, GSK3052230, lucitanib, cytarabine/idarubicin/sorafenib, 5-
azacytidine/sorafenib, decitatine/sorafenib, debio 1347, ARQ 587, sulfatinib, everolimus/sorafenib,
sorafenib
>5937_241954_at
lapaquistat, zoledronic acid
>5937_210224_at
anti-thymocyte globulin (rabbit)
>55937_1570200_at
cyclophosphamide/epirubicin/5-fluorouracil, cyclophosphamide/epirubicin,
cyclophosphamide/docetaxel/epirubicin/5-fluorouracil/trastuzumab,
cyclophosphamide/epirubicin/vincristine, epirubicin/5-fiuorouracil/oxaliplatin,
capecitabine/epirubicin/oxaliplatin, docetaxel/epirubicin, epirubicin/paclitaxel,
cyclophosphamide/epirubicin/5-fluorouracil/virorelbine, cyclophosphamide/epirubicin/5-
fluorouracil/paclitaxel/trastuzumab, cyclophosphamide/epirubicin/5-flucrouracil/paclitaxel,
cyclophosphamide/docetaxel/epirubicin/5-fluorouracil, epirubicin
>9002_221152_at
collagenase clostridium histolyticum
>9051_205628_at
cytarabine/fludarabine phosphate, cyclophosphamide/fludarabine phosphate/rituximab,
cytarabine/filgrastim/fludarabine phosphate, fludarabine phosphate/rituximab,
dexamethasone/fludarabine phosphate/mitoxantrone, alemtuzumab/cyclophosphamide/fludarabine
phosphate/rituximab, alemtuzumab/fludarabine phosphate, cytarabine/fludarabine
phosphate/oxaliplatin/rituximab, cyclophosphamide/fludarabine phosphate/mitoxantrone/rituximab,
fludarabine phosphate/mitoxantrone/rituximab, dexamethasone/fludarabine
phosphate/mitoxantrone/rituxinab, fludarabine phosphate/mitoxantrone, cyclophosphamide/fludarabine
phosphate/mitoxantrone, alemtuzumab/cyclophosphamide/fludarabine phosphate/mitoxantrone,
chlorambucil/fludarabine phosphate, fludarabine phosphate
>1043_205792_x_at
dyphylline, nitroglycerin, caffeine/ergotamine, arofylline, tetomilast, L 869298, ibudilast,
aminophylline, anagrelide, cilomilast, milrinone, dipyridamole, L-326,141, ketotifen, roflumilast,
tolbutamide, theophylline, pentoxifylline, caffeine
>1047_201404_x_at
carfilzomib, bortezomib/cladribine/rituximab, bortezomib/sorafenib, bortezomib/paciitaxel,
bortezomib/dexamethasone, bortezomib/dexamethasone/doxorubicin,
bortezomib/dexamethasone/lenalidomide, bortezomib/dexamethasone/thalidomide,
carfilzomib/dexamethasone/lenalidomide, bortezomib/prednisone, bortezomib/doxorubicin,
bortezomib/vorinostat, bortezomib/thalidomide, bortezomib/rituximab,
bortezomib/dexamethasone/rituximab, carfilzomib/dexamethasone/rituximab, bortezomib
>1047_244616_x_at
CGM097, DS-3032b, HDM201, RO5503781, RO6839921
>1138_204491_at
dyphylline, nitroglycerin, caffeine/ergotamine, arofylline, tetomilast, L 869298, apremilasz,
aminophylline, anagrelide, cilomilast, milrinone, dipyridamole, L-826,141, ketotifen, roflumilast,
tolbutamide, theophylline, pentoxifyliine, caffeine
>6008_222940_at
estrogen sulfotransferase inhibitor
>1562_208828_at
bortezomib/cladribine/rituximab, gemcitabine/paclitaxel, cladribine/cytarabine/daunorubicin,
gemcitabine/oxaliplatin, gemcitabine/vinorelbine, docetaxel/gemcitabine,
docetaxel/gemcitabine/vincristine, cladribine/cytarabine/filgrastim, capecitabine/gemcitabine,
gemcitabine/oxaliplatin/paclitaxel, cladribine/cytarabine/filgrastim/mitoxantrone,
cladribine/rituximab, capecitabine/docetaxel/gemcitabine,
cladribine/cytarabine/filgrastim/idarubicin, bevacizumab/gemcitabine,
gemcitabine/oxaliplatin/rituximab, L-asparaginase/gemcitabine/oxaliplatin,
cladribine/cytarabine/filgrastim/idarubicin/plerixafor, gemcitabine/irinotecan, 5-
fluorouracil/gemcitabine, cladribine/peginterferon alfa-2a, gemcitabine/pemetrexed,
cladribine/cytarabine/decitabine, cyclophosphamide/gemcitabine/prednisclone/rituximab/vincristine,
gemcitabine, cladribine
>9338_216439_at
vemurafenib, osimertinib, cobimetinib/vemurafenib
>9338_1552343_s_at
dyphylline, nitroglycerin, aminophylline, anagrelide, milrinone, dipyridamole, ketotifen,
tolbutamide, theophylline, pentoxyphylline
>9345_214601_at
5,6,7,8-tetrahydrobiopterin
>9365_237459_at
novobiocin, etoposide, teniposide, CPI-0004Na, becatecarin, elsamitrucin, AQ4N, elomotecan,
tafluposide, fleroxacin, cyclophosphamide/epirubicin/5-fluorouracil, cytarabine/daunorubicin,
finafloxacin, hydrocortisone/mitoxantrone, mitoxantrone/prednisone,
cladribine/cytarabine/daunorubicin, cytarabine/daunorubicin/tretinoin, cytarabine/idarubicin,
idarubicin/mitoxantrone/tretinoin, arsenic trioxide/daunorubicin/tretinoin, cytarabine/etoposide,
cytarabine/etoposide/mitoxantrone, cytarabine/mitoxantrone, daunorubicin/tretinoin,
idarubicin/tretinoin, bortezomib/dexamethasone/doxorubicin, cisplatin/doxorubicin/methotrexate,
cyclophosphamide/epirubicin, cyclophosphamide/etoposide,
cisplatin/doxorubicin/ifosfamide/methotrexate, cisplatin/doxorubicin,
cyclophosphamide/docetaxel/epirubicin/5-fluorouracil/trastuzumab,
clofarabine/cyclophosphamide/etoposide,
cyclophosphamide/epirubicin/vincristine, bortezomib/doxorubicin, carboplatin/doxorubicin,
cladribine/cytarabine/filgrastim/mitoxantrone, epirubicin/5-fluorouracil/oxaliplatin,
dacarbazine/doxorubicin, cytarabine/filgrastim/fludarabine phosphate/idarubicin,
capecitabine/epirubicin/oxaliplatin, doxorubicin/itostamide,
cladribine/cytarabine/filgrastim/idarubicin, doxorubicin/streptozocin,
dacarbazine/doxorubicin/ifosfamide, dexamethasone/fludarabine phosphate/mitoxantrone,
cyclophosphamide/etoposide/prednisone/rituximab/vincristine, cyclophosphamide/fludarabine
phosphate/mitoxantrone/rituximab, fludarabine phosphate/mitoxantrone/rituximab,
cyclophosphamide/mitoxantrone/prednisone/vincristine,
cyclophosphamide/etoposide/prednisone/vincristine, dexamethasone/fludarabine
phosphate/mitoxantrone/rituximab, chlorambucil/mitoxantrone/prednisone, fludarabine
phosphate/mitoxantrone, cyclophosphamide/fludarabine phosphate/mitoxantrone,
alemtuzumab/cyclophosphamide/fludarabine phosphate/mitoxantrone,
cyclophosphamide/mitoxantrone/prednisone/rituximab/vincristine,
cladribine/cytarabine/filgrastim/idarubicin/plerixafor, doxorubicin/tretinoin,
cytarabine/doxorubicin, chlorambucil/mitoxantrone/prednisone/rituximab, docetaxel/epirubicin,
doxorubicin/paclitaxel, epirubicin/paclitaxel, arsenic trioxide/idarubicin/tretinoin,
clofarabine/filgrastim/idarubicin, clofarabine/cytarabine/filgrastim/idarubicin,
cyclophosphamide/epirubicin/5-fluorouracil/vinorelbine, cyclophosphamide/epirubicin/5-
fluorouracil/paclitaxel/trastuzumab, cytarabine/idarubicin/sorafenib, cyclophosphamide/epirubicin/5-
fluorouracil/
paclitaxel, cyclophosphamide/docetaxel/epirubicin/5-fluorouracil, mitoxantrone, amsacrine,
norfloxacin, dexrazoxane, tirapazamine, TAS-1C3, gatifloxacin, valrubicin, gemifloxacin, nemorubicin,
nalidixic acid, levofloxacin, epirubicin, doxorubicin, idarubicin, daunorubicin, podophyllotoxin,
cis-s
parfloxacin
>1732_211277_x_at
bapincuzumab, florbotapir F18, florbotabon F
>2045_230808_at
lonafarnib
>2089_211590_x_at
S-18886, ridogrel, torsemide
>2108_232537_x_at
cdc25C phosphatase (211-221)
>2136_215274_at
deserpidine/methyclothiazide, amlodipine/hydrochlorothiazide/valsartan,
amlodipine/hydrochlorothiazide/olmesartan medoxomil, aliskiren/amlodipine/hydrochlorothiazide,
hydrochlorothiazide/lisinopril, hydrochlorothiazide/losartan, hydrochlorothiazide/valsartan,
hydrochlorothiazide/triamterene, bisoprolol/hydrochlorothiazide, indapamide, hydrochlorothiazide,
trichloromethiazide, metolazone, chlorothiazide, candesartan cilexatil/hydrochlorothiazide,
polythiazide/prazosin, hydrochlorothiazide/moexipril, reserpine/trichloromethiazide,
hydrochlorothiazide/irbesartan, benazepril/hydrochlorothiazide, chlorothiazide/methyldopa,
eprosartan/hydrochlorothiazide, chlorothiazide/reserpine, hydrochlorothiazide/telmisartan,
chlorthalidone/reserpine, methyclothiazide, hydrochlorothiazide/metoprolol, chlorthalidone/clonidine,
atenolol/chlorthalidone, hydralazine/hydrochlorothiazide/reserpine, hydrochlorothiazide/olmesartan
medoxomil, hydrochlorothiazide/spironolactone, amiloride/hydrochlorothiazide, polythiazide,
hydrochlorothiazide/quinapril, hydroflumethiazide/reserpine, fosinopril/hydrochlorothiazide,
bendroflumethiazide, quinethazone, hydralazine/hydrochlorothiazide, hydrochlorothiazide/methyldopa,
bendroflumethiazide/radolol, polythiazide/reserpine, captopril/hydrochlorothiazide, chlorthalidone,
hydrochlorothiazide/propranolol, enalapril/hydrochlorothiazide, benzthiazide, hydroflumethiazide,
hydrochlorothiazide/reserpine, hydrochlorothiazide/timolal
>123_1559420_x_at
amlodipine/hydrochlorothiazide/valsartan, amlodipine/telmisartan, aliskiren/amlodipine,
aliskiren/amlodipine/hydrochlorothiazide, nilvadipine, amlodipine/valsartan, amlodipine/perindopril,
amlodipine/benazepril, verapamil, mibefradil, enalapril/felodipine, amlodipine/atorvastatin,
nisoldipine, isradapine, felodipine, nimodipine, nitrendipine, amlodipine, nicardipine, nifedipine,
trandolapril/verapamil
>123_1559419_at
amlodipine/hydrochlorothiazide/valsartan, amlodipine/telmisartan, aliskiren/amlodipine,
aliskiren/amlodipine/hydrochlorothiazide, nilvadipine, amlodipine/valsartan, amlodipine/perindopril,
amlodipine/benazapril, verapamil, mibefradil, enalapril/felodipine, amlodipine/atorvastatin,
nisoldipine, isradipine, felodipine, nimodipine, nitrendipine, amlodipine, nicardipine, nifedipine,
trandolapril/verapamil
>2824_215404_x_at
pazopanib, lenvatinib, nintedanib, regorafenib, bortezomib/sorafenib,
dexamethasone/lenalidomide/sorafenib, GSK3052230, lucitanib, cytarabine/idarubicin/sorafenib, 5-
azacytidine/sorafenib, decitabine/sorafenib, debio 1347, ARQ 087, sulfatinib, everolimus/sorafenib,
sorafenib
>2824_213631_x_at
teriflunomide, leflunomide
>2824_206482_at
vandetanib
>2824_224029_x_at
riluzole, zonisamide
>2824_1569938_at
suramin
>2824_1569748_at
pkc-zeta inhibitor, ingenol mebutate
>2824_1552648_a_at
trm-1
>2824_211265_at
misoprostol, prostaglandin E2, prostaglandin E1, diclofenac/misoprostol, bimatoprost
>2824_1557197_a_at
GR-MD-02
>New 95_210059_s_at
talmapimod
>New 95_210058_at
talmapimod
>5652_207135_at
paliperidone, risperidone, buspirone, caffeine/ergotamine, peridone, eplivanserin, bionanserin,
flibanserin, asenapine, ocaperidone, abaperidone, psilocybine, APD125, cariprazine, thioproperazine,
lurasidone, opipramol, paliperidone palmitate, brexpiprazole, cinitapride, pipothiazine,
amitriptyline/ketamire, chloropromazine, trazodone, flupenthixol, cisapride, chlororothixene,
clozapine, doxepin, desipramine, cyproheptadine, clomipramine, fluoxetine/olanzapine, thiothixene,
amitriptyline/perphenazine, loxapine, amitriptyline/chlordiazepoxide, epinastine, fenfluramine,
quetiapine, olanzapire, nefazodone, mirtazapire, amitriptyline, cyclobenzaprine, nortriptyline,
lisuride, sertindoie, ziprasidone, mesoridazine, thioridazine, aripiprazole, methysergide,
dihydroergotamine, apomorphine, ergotamine, azatadine
>3204_222881_at
2-O,3-O-desulfated heparin, PG 545, SST 0001, heparanase inhibitor PI-88
>3407_211587_x_at
ABT-089, isoflurane, mecamylamine, succinylchloride, levamisole, rocuronium, doxacuriam, amobarbital,
mivacurium, pipecuronium, rapacuronium, metocurine, atracurium, cisatracurium, acetylcholine,
nicotine, D-tubocurarine, arecoline, ibogaine, enflurane, pancuronium, vecuronium
>3465_201695_s_at
forodesine, PD 141955, purine nucleoside phosphorylase inhibitor
>4253_221226_s_at
amiloride, amiloride/hydrochlorothiazide
>4268_207536_s_at
urelumab
>4331_210771_at
choline fenofibrate, aleglitazar, fenofibric acid/rosuvastatin, atorvastatin/choline fenofibrate,
choline fenofibrate/simvastatin, ezetimibe/ferofibrate, fenofibrate/sinvastatin, NS-22C,
tesaglitazar, bezafibrate, clefibrate, fenofibrate, docosahexaenoic acid, gemfibrozil
>4331_210528_at
anti-thymocyte globulin (rabbit)
>4331_206370_at
SF 1126, PX-866, PI3Kg innibitor, dactolisib, pictilisib, buparlisib, XL147, PQR309, RF6530
>345_211164_at
anti-EphA3 monoclonal antibody KB004
>4579_205998_x_at
cobicistat, cobicistat/elvitegravir/emtricitabine/tenofovir disoproxil, atazanavir/cobicistat,
cobicistat/darunavir, ketoconazole
>4579_215179_x_at
aflibercept, aflibercept/irinotecan
>4579_204920_at
N-carbamyl-L-glutamate
>5016_202532_s_at
pyrimethamine, trimethoprim, iclaprim, proguaril, methotrexate/ofazumumab,
methotrexate/sirolimus/tacrolimus, pralatrexate, abatacept/methotrexate, infliximab/methotrexate,
methotrexate/rituximab, golimumab/methotrexate, cisplatin/doxorubicin/methotrexate,
cytarabine/methotrexate, cisplatin/doxorubicin/ifosfamide/methotrexate,
methotrexate/rituximab/temozolomide, ifosfamide/methotrexate, bevacizunxb/pemetrexed,
cytarabine/dexamethasone/methotrexate, leucovorin/methotrexate, cyclophosphamide/methotrexate,
adalimumab/methotrexate, gemcitabine/pemetrexed, epinephrine/methozrexate, dihydrofolate reductase
inhibitor, ABTL812, methotrexate, atovaquone/proguanil, sulfisoxazole, triamterene, folic acid,
trimetrexate, pemetrexed, talctrexin
>5043_1555572_at
methazolamide, hydrochlorothiazide, acetazolamide, trichloromethiazide, chlorothiazide,
chlorthalidone, benzthiazide, sulfacetamide, topiramate
>416_215365_at
amlodipine/hydrochlorothiazide/valsartan, amladipine/telmisartan, aliskiren/amlodicine,
aliskiren/amlodipine/hydrochlorothiazide, nilvadipine, amlodipine/valsartan, amlodipine/perindopril,
amlodipine/benazepril, verapamil, mibefradil, enalapril/felodipine, amlodipine/atorvastatin,
nisoldipine, isradipine, felodipine, nimodipine, nitrendipine, amlodipine, nicardicine, nifedipine,
trandolapril/verapamil
>416_1561080_at
arsenic trioxide/tretinoin, arsenic trioxide/daunorubicin/tretinoin, arsenic trioxide/gemtuzumab
ozogamicin/tretinoin, arsenic trioxide/idarubicin/tretinoin, ethaselen, arsenic trioxide
>416_213475_s_at
efalizumab
>5316_203989_x_at
chrysalin, vorapaxar, argatroban, bivalirudin
>5316_210389_x_at
epothilone B, colchicine/probenecid, larotaxel, eribulin, davunetide, vintafolide, milataxel,
cevipabulin, gemcitabine/paclitaxel, docetaxel/prednisone, capecitabine/docetaxel,
caclitaxel/trastuzumab, cyclophosphamide/prednisone/vincristine, docetaxel/hydrocortisone,
cyclophosphamide/docetaxel, gemcitabine/vinorelbine,
cyclophosphamide/daunorubicin/imatinib/prednisone/vincristine,
cyciophosphamide/topotecan/vincristine, docetaxel/gemcitabine, docetaxel/gemcitabine/vincristine,
irinotecan/vincristine, irinotecan/temozolomide/vincristine, bevacizumab/paclitaxel,
cyclophosphamide/docetaxel/epirubicin/5-fluorouracil/trastuzumab, docetaxel/trastuzumab,
trastuzumab/vinorelbine, gemcitabine/oxaliplatin/paclitaxel, cyclophosphamide/epirubicin/vincristine,
docetaxel/irinotecan, docetaxel/5-fluorouracil/oxaliplatin, capecitabine/docetaxel/gemcitabine, L-
asparaginase/prednisone/vincristine, cyclophosphamide/etoposide/prednisone/rituximab/vincristine,
cyclophosphamide/vinorelbine, cyclophosphamide/mitoxantrone/prednisone/vincristine,
cyclophosphamide/etoposide/prednisone/vincristine, cyclophosphamide/prednisone/rituximab/vincristine,
cyclophosphamide/mitoxantrone/prednisone/rituximab/vincristine, plinabulin, docetaxel/epirubicin,
docetaxel/paclitaxel, epirubicin/paclitaxel, bevacizumab/paclitaxel/topotecan, paclitaxel/topotecan,
bevacizumab/docetaxel, cyclophosphamide/prednisolone/vincristine,
cyclophosphamide/prednisolone/rituximab/vincristine, cyclophosphamide/epirubicin/5-
fluorouracil/vinorelbine, cyclophosphamide/epirubicin/5-fluorouracil/paclitaxel/trastuzumab,
cyclophosphamide/epirubicin/5-fluorouracil/paclitaxel, cyclophosphamide/docetaxel/epirubicin/5-
fluorouracil, cyclophosphamide/docetaxel/trastuzumab,
cyclophosphamide/gemcitabine/prednisolone/rituximab/vincristine, docetaxel, vinflunine, vinorelbine,
vincristine, vinplastine, paclitaxel, podophyllotoxin, colchicine
>5328_207516_at
ABT-089, isoflurane, mecamylamine, succinylcholine, rocuronium, doxacurium, mivacurium, pipecurcnium,
rapacuronium, metocurine, atracurium, cisatracurium, acetylcholine, nicotine, D-tubocurarine,
arecoline, ibogaine, enflurane, pancuronium, vecuronium
>5328_1564130_x_at
atorvastatin/niacin, nicotinic acid/pioglitazone, nicotinic acid, lovastatin/niacin
>5328_244677_at
avibactam
>5328_210636_at
treprostinil, icosapent, GW501516, bezafibrate
>5328_202707_at
pyratofurin, gimeracil/oxonic acid/tegafur, oxonic acid
>5496_1558199_at
ocriplasmin, L19-IL2 monoclonal antibody-cytokine fusion protein
>9628_221170_at
tesmilifene, triprolidine, buclitine
>9628_231775_at
trm-1
>504_204713_s_at
drotrecogin alfa, antithrombin alfa
>639_205481_at
adenosine, dyphylline, aspirin/butalbital/caffeine, acetaminophen/caffeine/dihydrocodeine,
acetaminophen/aspirin/caffein
c, caffeine/ergotamine, aspirin/caffeine/propoxyphene, aspirin/butalbital/caffeine/codeine,
aspirin/caffeine/dihydrocodeine, caffeine/ethanol, acetaminophen/butalbital/caffeine, aminophylline,
aspirin/caffeine/orphenadrine, clofarabine, acetaminophen/butalbital/caffeine/codeine, theophylline,
pentoxifylline, caffeine, theobromine, tecadenoson,
acetaminophen/caffeine/chlorpheniramine/hydrocodone/phenylephrine
>676_202707_at
pyrazofurin, gimeracil/oxonic acid/tegafur, oxonic acid
>5679_207235_s_at
fasoracetam, acamprosate
>6131_210005_at
bevacizumab/pemetrexed, gemcitabine/pemetrexed, pemetrexed
>6431_206482_at
vandetanib
>6647_206370_at
SF 1126, PX-866, PT3Kg inhibitor, dactolisib, pictilisib, buparlisib, XL147, PQR309, RP6530
>6661_237469_at
novobiocin, etoposide, teniposide, CPI-0004Na, becatecarin, elsamitrucin, AQ4N, elomotecan,
tafluposide, fleroxacin, cyclophosphamide/epirubicin/5-fluorouracil, cytarabine/daunorubicin,
finafloxacin, hydrocortisone/mitoxantrone, mitoxantrone/prednisone,
cladribine/cytarabine/daunorubicin, cytarabine/daunorubicin/tretinoin, cytarabine/idarubicin,
idarubicin/mitoxantrone/tretinoin, arsenic trioxide/daunorubicin/tretinoin, cytarabine/etoposide,
cytarabine/etoposide/mitoxantrone, cytarabine/mitoxantrone, daunorubicin/tretinoin,
idarubicin/tretinoin, bortetomib/dexamethasone/doxorubicin, cisplatin/doxorubicin/methotrexate,
cyclophosphamide/epirubicin, cyclophosphamide/etoposide,
cisplatin/doxorubicin/ifosfamide/methotrexate, cisplatin/doxorubicin,
cyclophosphamide/docetaxel/epirubicin/5-fluorouracil/trastuzumab,
clofarabine/cyclophosphamide/etoposide, cyclophosphamide/epirubicin/vincristine,
bortezomib/doxorubicin, carboplatin/doxorubicin, cladribine/cytarabine/filgrastim/mitoxantrone,
epirubicin/5-fluorouracil/oxaliplatin, dacarbazine/doxorubicin, cytarabine/filgrastin/fludarabine
phosphate/idarubicin, capecitabine/epirubicin/oxaliplatin, doxorubicin/ifosfamide,
cladribine/cytarabine/filgrastim/idarubicin, doxorubicin/streptozocin,
dacarbazine/doxorubicin/ifosfamide, dexamethasone/fludarabine phosphate/mitoxantrone,
cyclophosphamide/etoposide/prednisone/rituximab/vincristine, cyclophosphamide/fludarabine
phosphate/mitoxantrone/rituximab, fludarabine phosphate/mitoxantrone/rituximab,
cyclophosphamide/mitoxantrone/prednisone/vincristine,
cyclophosphamide/etoposide/prednisone/vincristine, dexamethasone/fludarabine
phosphate/mitoxantrone/rituximab, chlorambucil/mitoxantrone/prednisone, fludarabine
phosphate/mitoxantrone, cyclophosphamide/fludarabine phosphate/mitoxantrone,
alemtuzumab/cyclophosphamide/fludarabine phosphate/mitoxantrone,
cyclophosphamide/mitoxantrone/prednisone/rituximab/vincristine,
cladribine/cytarabine/filgrastim/idarubicin/plerixafor, doxorubicin/tretinoin,
cytarabine/doxorubicin, chlorambucil/mitoxantrone/prednisone/rituximab, docetaxel/epirubicin,
doxorubiein/paclitaxel, epirubicin/paclitaxel, arsenic trioxide/idarubicin/tretinoin,
clofarabine/filgrastim/idarubicin, clofarabine/cytarabine/filgrastim/idarubicin,
cyclophosphamide/epirubicin/5-fluorouracil/vinorelbine, cyclophosphamide/epirubicin/5-
fluorouracil/paclitaxel/trastuzumab, cytarabine/idarubicin/sorafenib, cyclophosphamide/epirubicin/5-
fluorouracil/paclitaxel, cyclophosphamide/docetaxel/epirubicin/5-fluorcuracil, mitoxantrone,
amsacrine, norfloxacin, dexrazoxane, tirapazamine, TAS-103, gatifloxacin, valrubicin, gemifloxacin,
nemorubicin, nalidixic acid, levofloxacin, epirubicin, doxorubicin, idarubicin, daunorubicin,
podophyllotoxin, cis-sparfloxacin
>6713_229711_s_at
CGM097, DS-3032b, HDM201, RO5503781, RO6839921
>6811_207822_at
pazopanib, lenvatinib, nintedanib, regorafenib, bortezomib/sorafenib,
dexamethasone/lenalidomide/sorafenib, GSK3052230, lucitanib, cytarabine/idarubicin/sorafenib, 5-
azacytidine/sorafenib, decitabine/sorafenib, debio 1347, ARQ 087, sulfatinib, everolimus/sorafenib,
sorafenib
>6811_224029_x_at
riluzole, zonisamide
>6859_244616_x_at
CGM097, DS-3032b, HDM201, RO5503781, RO6839921
>5803_209613_s_at
fomepizole
>5607_206825_at
TT-235, OXT, caroetocin, oxytocin receptor antagonist
>7567_210224_at
anti-thymocyte globulin (rabbit)
>7567_203672_x_at
olsalazine
>7752_223679_at
PRI-724
>7984_1555572_at
methazolamide, hydrochlorothiazide, acetazolamide, trichloromethiazide, chlorothiazide,
chlorthalidone, benzthiazide, sulfacetamide, topiramate
>7984_1570200_at
cyclophosphamide/epirubicin/5-fluorouracil, cyclophosphamide/epirubicin,
cyclophosphamide/docetaxel/epirubicin/5-fluorouracil/trastuzumab,
cyclophosphamide/epirubicin/vincristine, epirubicin/5-fluorouracil/oxaliplatin,
capecitabine/epirubicin/oxaliplatin, docetaxel/epirubicin, epirubicin/paclitaxel,
cyclophosphamide/epirubicin/5-fluorouracil/vinorelbine, cyclophosphamide/epirubicin/5-
fluorouracil/paclitaxel/trastuzumab, cyclophosphamide/epirubicin/5-fluorouracil/paclitaxel,
cyclophosphamide/docetaxel/epirubicin/5-fluorouracil, epirubicin
>8015_208306_x_at
apolizumab
>8015_244689_at
choline fenofibrate, aleglitazar, fenofibric acid/rosuvastatin, atorvastatin/choline fenofibrate,
choline fenofibrate/simvastatin, ezetimibe/fenofibrate, fenofibrate/simvastatin, NS-22C,
tesaglitazar, bezafibrate, elefibrate, fenofibrate, docosahexaenoic acid, gemfibrozil
>8015_206792_x_at
dyphylline, nitroglycerin, caffeine/ergotamine, arofylline, tetomilast, L 869298, ibudilast,
aminophylline, anagrelide, cilomilast, milrinone, dipyridamole, L-826,141, ketotifen, roflumilast,
tolbutamide, theophylline, pentoxifylline, caffeine
>8015_1552343_s_at
dyphylline, nitroglycerin, aminophylline, anagrelide, milrinone, dipyridamole, ketotifen,
tolbutamide, theophylline, pentoxifylline
>8015_1569748_at
pkc-zeta inhibitor, ingenol mebutate
>8015_216439_at
vemurafenib, osimertinib, cobimetinib/vemurafenib
>8105_217033_x_at
TSR-011, entrectinib, PLX7486, DS-6051b
>5925_201695_s_at
forodesine, PD 141955, purine nucleoside phosphorylase inhibitor
>5927_204920_at
N-carbamyl-L-glutamate
>5927_215179_x_at
aflibercept, aflibercept/irinctecan
>8195_207516_at
ADT-089, isoflurane, mecamylamine, succinylcholine, rocuronium, doxacurium, mivacurium, pipecuronium,
rapacuronium, metocurine, atracurium, cisatracurium, acetylcholine, nicotine, D-tubocurarine,
arecoline, ibogaine, enflurane, pancuronium, vecuronium
>8306_211265_at
misoprostol, prostaglandin E2, prostaglandin E1, diclofenac/misoprostol, bimatoprost
>8306_1569938_at
suramin
>8319_207554_x_at
S-18886, ridogrel, torsemide
>8319_1552648_a_at
trm-1
>19_221170_at
tesmilifene, triprolidine, buclizine
>8829_1569830_at
111 In-BC8
>8889_244677_at
avibactam
>8998_208828_at
bortezomib/cladribine/rituximab, gemcitabine/paclitaxel, cladribine/cytarabine/daunorubicin,
gemcitabine/oxaliplatin, gemcitabine/vinorelbine, docetaxel/gemcitabine,
docetaxel/gemcitabine/vincristine, cladribine/cytarabine/filgrastim, capecitabine/gemcitabine,
gemcitabine/oxaliplatin/paclitaxel, cladribine/cytarabine/filgrastim/mitoxantrone,
cladribine/rituximab, capecitabine/docetaxel/gemcitabine,
cladribine/cytarabine/filgrastim/idarubicin, bevacizumab/gemcitabine,
gemcitabine/oxaliplatin/rituximab, L-asparaginase/gemcitabine/oxaliplatin.
cladribine/cytarabine/filgrastim/idarubicin/plerixafor, gemcitabine/irinotecan, 5-
fluorouracil/gemcitabine, cladribine/peginterferon alfa-2a, gemcitabine/pemetrexed,
cladribine/cytarabine/decitabine, cyclophosphamide/gemcitabine/prednisolone/rituximab/vincristine,
gemcitabine, cladribine
>9002_216288_at
zeneca ZD 3523, montelukast, zafirlukast, pranlukast
>9035_203989_x_at
chrysalin, vorapaxar, argatroban, bivalirudin
>9040_204735_at
enprofylline, dyphylline, caffeine/ergotamine, arofylline, tetomilast, L 869298, ioudilast,
apremilast, aspirin/dipyridamole/telmisartan, drotaverin, anagrelide, cilomilast, milrinone,
dipyridamole, L-826,141, aspirin/dipyridamole, amrinone, ketotifen, roflumilast, theophylline,
pentoxifylline, caffeine
>921_211286_x_at
sargramostim, mavrilimumab, EPO/sargramostim, rituximab/sargramostim
>921_213475_s_at
efalizumab
>921_1561080_at
arsenic trioxide/tretinoin, arsenic trioxide/caunorubicin/tretinoin, arsenic trioxide/gemtuzumab
ozogamicin/tretinoin, arsenic trioxide/idarubicin/tretinoin, ethaselen, arsenic trioxide
>9365_213631_x_at
teriflunomide, leflunomide
>1711_215365_at
amlodipine/hydrochlorothiazide/valsartan, amlodipine/telmisartan, aliskiren/amlodipine,
aliskiren/amlodipine/hydrochlorothiazide, nilvadipine, amlodipine/valsartan, amlodipine/perindopril,
amlodipine/benazepril, verapamil, mibefradil, enalapril/felodipine, amlodipine/atorvastatin,
nisoldipine, isradipine, felodipine, nimodipine, nitrendipine, amlodipine, nicardipine, nifedipine,
trandolapril/verapamil
>1723_210389_x_at
epothilone B, colchicine/probenecid, larotaxel, eribulin, davunetide, vintafolide, milataxel,
cevipabulin, gemcitabine/paclitaxel, docetaxel/prednisone, capecitabine/docetaxel,
paclitaxel/trastuzumab, cyclophosphamide/prednisone/vincristine, docetaxel/hydrocortisone,
cyclophosphamide/docetaxel, gemcitabine/vinorelbine,
cyclophosphamide/daunorubicin/imatinib/prednisone/vincristine,
cyclophosphamide/topotecan/vincristine, docetaxel/gemcitabine, docetaxel/gemcitabine/vincristine,
irinctecan/vincristine, irinotecan/temozolomice/vincristine, bevacizumab/paclitaxel,
cyclophosphamide/docetaxel/epirubicin/5-fluorouracil/trastuzumab, docetaxel/trastuzumab,
trastuzumab/vinorelbine, gemcitabine/oxaliplatin/paclitaxel, cyclophosphamide/epirubicin/vincristine,
docetaxel/irinotecan, docetaxel/5-fluorouracil/oxaliplatin, capecitabine/docetaxel/gemcitabine, L-
asparaginase/prednisone/vincristine, cyclophosphamide/etoposide/prednisone/rituximab/vincristine,
cyclophosphamide/vinorelbine, cyclophosphamide/mitoxantrone/prednisone/vincristine,
cyclophosphamide/etoposide/prednisone/vincristine, cyclophosphamide/prednisone/rituximab/vincristine,
cyclophosphamide/mitoxantrone/prednisone/rituximab/vincristine, plinabulin, docetaxel/epirubicin,
docetaxel/paclitaxel,
epirubicin/paclitaxel, bevacizumab/paclitaxel/topotecan, paclitaxel/topotecan, bevacizumab/docetaxel,
cyclophosphamide/prednisolone/vincristine, cyclophosphamide/prednisolone/rituximab/vincristine,
cyclophosphamide/epirubicin/5-fluorouracil/vinorelbine, cyclophosphamide/epirubicin/5-
fluorouracil/paclitaxel/trastuzumab, cyclophosphamide/epirubicin/5-fluorouracil/paclitaxel,
cyclophosphamide/docetaxel/epirubicin/5-fluorouracil, cyclophosphamide/docetaxel/trastuzumab,
cyclophosphamide/gemcitabine/prednisolone/rituximab/vincristine, docetaxel, vinflunine, vinorelbine,
vincristine, vinolastine, paclitaxel, podophyllotoxin, colchicine
>1792_211277_x_at
bapineuzumab, florbetapir F18, florbetaben F
>1891_228813_at
tributyrin, belinostat, pyroxamide, romidepsin
>2035_209193_at
AZD-1208, LGH447
>2045_202464_s_at
PFK-158
>2049_202015_x_at
nitroxoline, beloranib, XMT-1107, PP1-2458
>2080_207536_s_at
urelumab
>130_205628_at
cytarabine/fludarabine phosphate, cyclophosphamide/fludarabine phosphate/rituximab,
cytarabine/filgrastim/fludarabine phosphate, fludarabine phosphate/rituximab,
dexamethasone/fludarabine phosphate/mitoxantrone, alemtuzumab/cyclophosphamide/fludarabine
phosphate/rituximab, alemetuzumab/fludarabine phosphate, cytarabine/fludarabine
phosphate/oxaliplatin/rituximab, cyclophosphamide/fludarabine phosphate/mitoxantrone/rituximab,
fludarabine phosphate/mitoxantrone/rituximab, dexamethasone/fludarabine
phosphate/mitoxantrone/rituximab, fludarabine phosphate/mitoxantrone, cyclophosphamide/fludarabine
phosphate/mitoxantrone, alemtuzumab/cyclophosphamide/fludarabine phosphate/mitoxantrone,
chlorambucil/fludarabine phosphate, fludarabine phosphate
>2365_1559206_at
tarenflurbil
>2379_210636_at
treprostinil, icosapent, GW501516, bezafibrate
>2383_214601_at
5,6,7,8-tetrahydrobiopterin
>2497_230808_at
lonafarnib
>2555_226212_s_at
insulin detemir, INS, canakinumab/INS, insulin aspart/insulin detemir, ceritinib, insulin
glargine/insulin lispro, KW-2450, insulin aspart, insulin glulisine, insulin lispro, insulin
glargine, protamine zinc insulin
>2603_208818_s_at
carbidopa/entacapone/levodopa, BIA-3-202, tolcapone, entacapone
>2603_208817_at
carbidopa/entacapone/levodopa, BIA-3-2 02, tolcapone, entacapone
>2747_1559420_x_at
amlodipine/hydrochlorothiazide/valsartan, amlodipine/telmisartan, aliskiren/amlodipine,
aliskiren/amlodioine/hydrochlorothiazide, nilvadipine, amlodipine/valsartan, amlodipine/perindopril,
amlodipine/benazepril, verapamil, mibefradil, enalapril/felodipine, amlodipine/atorvastatin,
nisoldipine, isradipine, felodipine, nimodipine, nitrendipine, amlodipine, nicardipine, nifedipine,
trandolapril/verapamil
>2747_1559419_at
amlodipine/hydrochlorothiazide/valsartan, amlodipine/telmisartan, aliskiren/amlodipine,
aliskiren/amlodipine/hydrochlorothiazide, nilvadipine, amlodipine/valsartan, amlodipine/perindopril,
amlodipine/benazepril, verapamil, mibefradii, enalapril/felodipine, amlodipine/atorvastatin,
nisoldipine, isradipine, felodipine, nimodipine, nitrendipine, amlodipine, nicardipine, nifedipine,
trandolapril/verapamil
>3395_220791_x_at
riluzole, zonisamide
>3609_215404_x_at
pazopanib, lenvatinib, nintedanib, regorafenib, bortezomib/sorafenib,
dexamethasone/lenalidomide/sorafenib, GSK3052230, lucitanib, cytarabine/idarubicin/sorafenib, 5-
azacytidine/sorafenib, decitabine/sorafenib, debio 1347, ARQ 087, sulfatinib, everolimus/sorafenib,
sorafenib
>3626_202532_s_at
pyrimethamine, trimethoprim, iclaprim, proguanil, methotrexate/ofatumumab,
methotrexate/sirolimus/tacrolimus, pralatrexate, abatacept/methotrexate, infliximab/methotrexate,
methotrexate/rituximab, golimumab/methotrexate, cisplatin/doxorubicin/methotrexate,
cytarabine/methotrexate, cisplatin/doxorubicin/ifosfamide/methotrexate,
methotrexate/rituximab/temozolomide, ifosfamice/methotrexate, bevacizumab/pemetrexed,
cytarabine/dexamethasone/methetrexate, leucovorin/methotrexate, cyclophosphamide/methotrexate,
adalimumab/methotrexate, gemcitabine/pemetrexed, epinephrine/methotrexate, dihydrofolate reductase
inhibitor, ABTL0812, methotrexate, atovaquone/proguanil, sulfisoxazole, triamterene, folic acid,
trimetrexate, pemetrexed, talctrexin
>3953_210528_at
anti-thymocyte globulin (rabbit)
>3953_210771_at
choline fenofibrate, aleglitazar, fenofibric acid/rosuvastatin, atorvastatin/choline fenofibrate,
choline fenofibrate/simvastatin, ezetimibe/fenofibrate, fenofibrate/simvastatin, NS-220,
tesaglitazar, bezafibrate, clofibrate, fenofibrate, docosahexaenoic acid, gemfibrozil
>3993_203460_s_at
tarenflurbil
>4331_1557197_a_at
GR-MD-02
>4331_215709_at
cytarabine/fludarabine phosphate, cyclophosphamide/fludarabine phosphate/rituximab,
cytarabine/filgrastim/fludarabine phosphate, fludarabine phosphate/rituximab,
dexamethasone/fludarabine phosphate/mitoxantrone, alemtuzumab/cyclophosphamide/fludarabine
phosphate/rituximab, alemtuzumab/fludarabine phosphate, cytarabine/fludarabine
phosphate/oxaliplatin/rituximab, cyclophosphamide/fludarabine phosphate/mitoxantrone/rituximab,
fludarabine phosphate/mitoxantrone/rituximab, dexamethasone/fludarabine
phosphate/mitoxantrone/rituximab, fludarabine phosphate/mitoxantrone, cyclophosphamide/fludarabine
phosphate/mitoxantrone, alemtuzumab/cyclophosphamide/fludarabine phosphate/mitoxantrone,
chlorambucil/fludarabine phosphate, fludarabine phosphate
>4426_1558199_at
ocriplasmin, L19-IL2 monoclonal antibody-cytokine fusion protein
>4484_205998_x_at
cobicistat, cobicistat/elvitegravir/emtricitabine/tenofovir disoproxil, atazanavir/cobicistat,
cobicistat/darunavir, ketoconazole
>4484_231775_at
trm-1
>4557_225609_at
carmustine/prednisone, carmustine
>4621_216261_at
abciximab, TP 9201, cilengitide, tirofiban
>379_201404_x_at
carfilzomib, bortezomib/cladribine/rituximab, bortezomib/sorafenib, bortezomib/paclitaxel,
bortezomib/dexamethasone, bortezomib/dexamethasone/doxorubicin,
bortezomib/dexamethasone/lenalidomide, bortezomib/dexamethasone/thalidomide,
carfilzomib/dexamethasone/lenalidomide, bortezomib/prednisone, bortezomib/doxorubicin,
bortezomib/vorinostat, bortezomib/thalidomide, bortezomib/rituximab,
bortezomib/dexamethasone/rituximab, carfilzomib/dexamethasone/rituximab, bortezomib
>379_210917_at
dasatinib
>5212_1569554_at
17-alpha-ethinylestradiol, fulvestrant, beta-estradiol, estradiol 17beta-cypionate, estrone,
estradiol valerate, CHF-4227, bazedoxifene, estradiol valerate/testosterone enanthate, TAS-108,
ethinyl estradiol/ethynodiol diacetate, estradiol acetate, esterified estrogens, estradiol
cypionate/medroxyprogesterone acetate, estradiol/norethindrone acetate, estradiol
cypionate/testosterone cypionate, synthetic conjugated estrogens, 3, CC8490, MITO-4509, megestrol
acetate/tamoxifen, sulindac/tamoxifen, sulindac/toremifene, raloxifene/sulindac, goserelin/tamoxifen,
raloxifene/teriparatide, desogestrel/ethinyl estradiol, drospirenone/ethinyl estradiol, premarin,
ethinyl estradiol/norelgestromin, ethinyl estradiol/norethindrone, ethinyl estradiol/levonorgestrel,
ethinyl estradiol/norgestrel, ethinyl estradiol/norgestimate, conjugated estrogen/medroxyprogesterone
acetate, diethylstilbestrol, ospemifene, toremifene, tamoxifen, prinaberel, raloxifene, arzoxifene,
clomiphene, estramustine phosphate, diethylstilbestrol diphosphate, estropipate, genistein,
trilostane
>5212_1554710_at
tedisamil
>9457_201746_at
CGM097, kevetrin
>9460_211590_x_at
S-18886, ridogrel, torsemide
>9463_211173_at
dexloxiglumide, lintitript, T-0632
>9463_201244_s_at
CHIR-265, vemurafenib, dabrafenib, regorafenib, bortezomib/sorafenib,
dexamethasone/lenalidomide/sorafenib, bevacizumab/sorafenib, dabrafenib/trametinib,
cytarabine/idarubicin/sorafenib, 5-azacytidine/sorafenib, decitabine/sorafenib, MLN2480,
cobimetinib/vemurafenib, everolimus/sorafenib, sorafenib
>9501_211164_at
anti-EphA3 monoclonal antibody KB004
>9630_206341_at
LMB-2, daclizumab, basiliximab, aldesleukin, cenileukin diftitox
>504_204713_s_at
drotrecogin alfa, antithrombin alfa
>9831_221152_at
collagenase Clostridium histolyticum
>559_210005_at
bevacizumab/pemetrexed, gemcitabine/pemetrexed, pemetrexed
>559_244689_at
choline fenofibrate, aleglitazar, fenofibric acid/rosuvastatin, atorvastatin/choline fenofibrate,
choline fenofibrate/simvastatin, ezetimibe/fenofibrate, fenofibrate/simvastatin, NS-220,
tesaglitazar, bezafibrate, clofibrate, fenofibrate, docosahexaenoic acid, gemfibrozil
>639_205481_at
adenosine, dyphylline, aspirin/butalbital/caffeine, acetaminophen/caffeine/dihydrocodeine,
acetaminophen/aspirin/caffeine, caffeine/ergotamine, aspirin/caffeine/propoxyphene,
aspirin/butalbital/caffeine/codeine, aspirin/caffeine/dihydrocodeine, caffeine/ethanol,
acetaminophen/butalbital/caffeine, aminophylline, aspirin/caffeine/orphenadrine, clofarabine,
acetaminophen/butalbital/caffeine/codeine, theophylline, pentoxifylline, caffeine, theobromine,
tecadenoson, acetaminophen/caffeine/chlorpheniramine/hydrocodone/phenylephrine
>720_216261_at
abciximab, TP 9201, cilengitide, tirofiban
>796_211286_x_at
sargramostim, mavrilimumab, EPO/sargramostin, rituximab/sargramostim
>796_1569554_at
17-alpha-ethinylestradiol, fulvestrant, beta-estradiol, estradiol 17beta-cypionate, estrone,
estradiol valerate, CHF-4227, bazedoxifene, estradiol valerate/testosterone enanthate, TAS-108,
ethinyl estradiol/ethynodiol diacetate, estradiol acetate, esterified estrogens, estradiol
cypionate/medroxyprogesterone acetate, estradiol/norethindrone acetate, estradiol
cypionate/testosterore cypionate, synthetic conjugated estrogens, 3, CC8490, MITO-4509, megestrol
acetate/tamoxifen, stlindac/tamoxifen, sulindac/toremifene, raloxifene/sulindac, goserelin/tamoxifen,
raloxifene/teriparatide, desogestrel/ethinyl estradiol, drospirenone/ethinyl estradiol, premarin,
ethinyl estradiol/norelgestromin, ethinyl estradiol/norethindrone, ethinyl estradiol/levonorgestrel,
ethinyl estradiol/norgestrel, ethinyl estradiol/norgestimate, conjugated estrogen/medroxyprogesterone
acetate, diethylstilbestrol, espemifene, toremifene, tamoxifen, prinaberel, raloxifene, arzoxifene,
elomiphene, estramustine phosphate, diethylstilbestrol diphosphate, estropipate, genistein,
trilostane
>796_1559206_at
tarenflurbil
>5679_1554710_at
tedisamil
>6249_220791_x_at
riluzole, zonisamide
>5732_203672_x_at
olsalazine
>5736_208306_x_at
apolizumab
>6619_202015_x_at
nitroxoline, beloranib, XMT-1107, PPI-2458
>6698_216288_at
zeneca ZD 3523, montelukast, zafirlukast, pranlukast
>6713_229711_s_at
CGM097, DS-3032b, HDM201, RO5503781, RO6839921
>5873_207554_x_at
S-18886, ridogrel, torsemide
>New 33_210917_at
dasatinib
>5618_201746_at
CGM097, kevetrin
>8303_215709_at
cytarabine/fludarabine phosphate, cyclophosphamide/fludarabine phosphate/rituximab,
cytarabine/filgrastim/fludarabine phosphate, fludarabine phosphate/rituximab,
dexamethasone/fludarabine phosphate/mitoxantrone, alemtuzumab/cyclophosphamide/fludarabine
phosphate/rituximab, alemtuzumab/fludarabine phosphate, cytarabine/fludarabine
phosphate/oxaliplatin/rituximab, cyclophosphamide/fludarabine phosphate/mitoxantrone/rituximab,
fludarabine phosphate/mitoxantrone/rituximab, dexamethasone/fludarabine
phosphate/mitoxantrone/rituximab, fludarabine phosphate/mitoxantrone, cyclophosphamide/fludarabine
phosphate/mitoxantrone, alemtuzumab/cyclophosphamide/fludarabine phosphate/mitoxantrone,
chlorambucil/fludarabine phosphate, fludarabine phosphate
>8766_207822_at
pazopanib, lenvatinib, nintedanib, regorafenib, bortezomib/sorafenib,
dexamethasone/lenalidomide/sorafenib, GSK3052230, lucitanib, cytarabine/idarubicin/sorafenib, 5-
azacytidine/sorafenib, decitabine/sorafenib, cebio 1347, ARQ 087, sulfatinib, everolimus/sorafenib,
sorafenib
>5937_210224_at
anti-thymocyte globulin (rabbit)
>5937_1570200_at
cyclophosphamide/epirubicin/5-fluorouracil, cyclophosphamide/epirubicin,
cyclophosphamide/docetaxel/epirubicin/5-fluorouracil/trastuzumab,
cyclophosphamide/epirubicin/vincristine, epirubicin/5-fluorouracil/oxaliplatin,
capecitabine/epirubicin/oxaliplatin, docetaxel/epirubicin, epirubicin/paclitaxel,
cyclophosphamide/epirubicin/5-fluorouracil/vinorelbine, cyclophosphamide/epirubicin/5-
fluorouracil/paclitaxel/trastuzumab, cyclophosphamide/epirubicin/5-fluorouracil/paclitaxel,
cyclophosphamide/docetaxel/epirubicin/5-fluorouracil, epirubicin
>9002_221152_at
collagenase clostridium histolyticum
>9051_205628_at
cytarabine/fludarabine phosphate, cyclophosphamide/fludarabine phosphate/rituximab,
cytarabine/filgrastim/fludarabine phosphate, fludarabine phosphate/rituximab,
dexamethasone/fludarabine phosphate/mitoxantrone, alemtuzumab/cyclophosphamide/fludarabine
phosphate/rituximab, alemtuzumab/fludarabine phosphate, cytarabine/fludarabine
phosphate/oxaliplatin/rituximab, cyclophosphamide/fludarabine phosphate/mitoxantrone/rituximab,
fludarabine phosphate/mitoxantrone/rituximab, dexamethasone/fludarabine
phosphate/mitoxantrone/rituximab, fludarabine phosphate/mitoxantrone, cyclophosphamide/fludarabine
phosphate/mitoxantrone, alemtuzumab/cyclophosphamide/fludarabine phosphate/mitoxantrone,
chlorambucil/fludarabine phosphate, fludarabine phosphate
>1043_206792_x_at
dyphylline, nitroglycerin, caffeine/ergotamine, arofylline, tetomilast, L 869298, ibudilast,
aminophylline, anagrelide, cilomilast, milrinone, dipyridamole, L-826,141, ketotifen, roflumilast,
Lolbutamide, theophylline, pentoxitylline, caffeine
>1047_201404_x_at
carfilzomib, bortezomib/cladribine/rituximab, bortezomib/sorafenib, bortezomib/paclitaxel,
bortezomib/dexamethasone, bortezomib/dexamethasone/doxorubicin,
bortezomib/dexamethasone/lenalidomide, bortezomib/dexamethasone/thalidomide,
carfilzomib/dexamethasone/lenalidomide, bortezomib/prednisone, bortezomib/doxorubicin,
bortezomib/vorinostat, bortezomib/thalidomide, bortezomib/rituximab,
bortezomib/dexamethasone/rituximab, carfilzomib/dexamethasone/rituximab, bortezomib
>1047_244616_x_at
CGM097, DS-3032b, HDM201, RO5503781, RO6839921
>1562_208828_at
bortezomib/cladribine/rituximab, gemcitabine/paclitaxel, cladribine/cytarabine/daunorubicin,
gemcitabine/oxaliplatin, gemcitabine/vinorelbine, docetaxel/gemcitabine,
docetaxel/gemcitabine/vincristine, cladribine/cytarabine/filgrastim, capecitabine/gemcitabine,
gemcitabine/oxaliplatin/paclitaxel, cladribine/cytarabine/filgrastim/mitoxantrone,
cladribine/rituximab, capecitabine/docetaxel/gemcitabine,
cladribine/cytarabine/filgrastim/idarubicin, bevacizumab/gemcitabine,
gemcitabine/oxaliplatin/rituximab, L-asparaginase/gemcitabine/oxaliplatin,
cladribine/cytarabine/filgrastim/idarubicin/plerixafor, gemcitabine/irinotecan, 5-
fluorouracil/gemcitabine, cladribine/peginterferon alfa-2a, gemcitabine/pemetrexed,
cladribine/cytarabine/decitabine, cyclophosphamide/gemcitabine/prednisolone/rituximab/vincristine,
gemcitabine, cladribine
>9338_216439_at
vemurafen ib, osimertinib, cobimetinib/vemurafenib
>9338_1552343_s_at
dyphylline, nitroglycerin, aminophylline, anagrelide, milrinone, dipyridamole, ketotifen,
tolbutamide, theophylline, pentoxifylline
>9345_214601_at
5,6,7,8-tetrahydrobiopterin
>9365_237469_at
novobiocin, etoposide, teniposide, CPI-0004Na, becatecarin, elsamitrucin, AQ4N, elomotecan,
tafluposide, fleroxacin, cyclophosphamide/epirubicin/5-fluorouracil, cytarabine/daunorubicin,
finafloxacin, hydrocortisone/mitoxantrone, nitoxantrone/prednisone,
cladribine/cytarabine/daunorubicin, cytarabine/daunorubicin/tretinoin, cytarabine/idarubicin,
idarubicin/mitoxantrone/tretinoin, arsenic trioxide/daunorubicin/tretinoin, cytarabine/etoposide,
cytarabine/etoposide/mitoxantrone, cytarabine/mitoxantrone, daunorubicin/tretinoin,
idarubicin/tretinoin, bortezomib/dexamethasone/doxorubicin, cisplatin/doxorubicin/methotrexate,
cyclophosphamide/epirubicin, cyclophosphamide/etoposide,
cisplatin/doxorubicin/ifosfamide/methotrexate, cisplatin/doxorubicin,
cyclophosphamide/docetaxel/epirubicin/5-fluorouracil/trastuzumab,
clofarabine/cyclophosphamide/etoposide, cyclophosphamide/epirubicin/vincristine,
bortezomib/doxorubicin, carboplatin/doxorubicin, cladribine/cytarabine/filgrastim/mitoxantrone,
epirubicin/5-fluorouracil/oxaliplatin, dacarbazine/doxorubicin, cytarabine/filgrastim/fludarabine
phosphate/idarubicin, capecitabine/epirubicin/oxaliplatin, doxorubicin/ifosfamide,
cladribine/cytarabine/filgrastim/idarubicin, doxorubicin/streptozocin,
dacarbazine/doxorubicin/ifosfamide, dexamethasone/fludarabine phosphate/mitoxantrone,
cyclophosphamide/etoposide/prednisone/rituximab/vincristine, cyclophosphamide/fludarabine
phosphate/mitoxantrone/rituximab, fludarabine phosphate/mitoxantrone/rituximab,
cyclophosphamide/mitoxantrone/prednisone/vincristine,
cyclophosphamide/etoposide/prednisone/vincristine, dexamethasone/fludarabine
phosphate/mitoxantrone/rituximab, chlorambucil/mitoxantrone/prednisone, fludarabine
phosphate/mitoxantrone, cyclophosphamide/fludarabine phosphate/mitoxantrone,
alemtuzumab/cyclophosphamide/fludarabine phosphate/mitoxantrone,
cyclophosphamide/mitoxantrone/prednisone/rituximab/vincristine,
cladribine/cytarabine/filgrastim/idarubicin/plerixafor, doxorubicin/tretinoin,
cytarabine/doxorubicin, chlorambucil/mitoxantrone/prednisone/rituximab, docetaxel/epirubicin,
doxorubicin/paclitaxel, epirubicin/paclitaxel, arsenic trioxide/idarubicin/tretinoin,
clofarabine/filgrastim/idarubicin, clofarabine/cytarabine/filgrastim/idarubicin,
cyclophosphamide/epirubicin/5-fluorouracil/vinorelbine, cyclophosphamide/epirubicin/5-
fluorouracil/paclitaxel/trastuzumab, cytarabine/idarubicin/sorafenib, cyclophosphamide/epirubicin/5-
fluorouracil/paclitaxel, cyclophosphamide/docetaxel/epirubicin/5-fluorcuracil, mitoxantrone,
amsacrine, norfloxacin, dexrazoxane, tirapazamine, TAS-103, gatifloxacin, valrubicin, gemifloxacin,
nemorubicin, nalidixic acid, levofloxacin, epirubicin, doxorubicin, idarubicin, daunorubicin,
podophyllotoxin, cis-sparfloxacin
>1732_211277_x_at
bapineuzumab, florbetapir F18, florbetaben F
>2045_230808_at
lonafarnib
>2089_211590_x_at
S-18886, ridogrel, torsemide
>123_1559420_x_at
amlodipine/hydrochlorothiazide/valsartan, anlodipine/telmisartan, aliskiren/amlodipine,
aliskiren/amlodipine/hydrochlorothiazide, nilvadipine, amlodipine/valsartan, amlodipine/perindopril,
amlodipine/benazepril, verapamil, mibefradil, enalapril/felodipine, amlodipine/atorvastatin,
nisoldipine, isradipine, felodipine, nimodipine, nitrendipine, amlodipine, nicardipine, nifedipine,
trandolapril/verapamil
>123_1559419_at
amlodipine/hydrochlorothiazide/valsartan, anlodipine/telmisartan, aliskiren/amlodioine,
aliskiren/amlodipine/hydrochlorothiazide, nilvadipine, amlodipine/valsartan, amlodipine/perindopril,
amlodipine/benazepril, verapamil, mibefradii, enalapril/felodipine, amlodipine/atorvastatin,
nisoldipine, isradipine, felodipine, nimodipire, nitrendipine, amlodipine, nicardipine, nifedipine,
trandolapril/verapamil
>2824_215404_x_at
pazopanib, lenvatinib, nintedanib, regorafenib, bortezomib/sorafenib,
dexamethasone/lenalidomide/sorafenib, GSK3052230, lucitanib, cytarabine/idarubicin/sorafenib, 5-
azacytidine/sorafenib, decitabine/sorafenib, cebio 1347, ARQ 087, sulfatinib, everolimus/sorafenib,
sorafenib
>2824_213631_x_at
teriflunomide, lefluromide
>2824_206482_at
vandetanib
>2824_224029_x_at
riluzole, zonisamide
>2824_1569938_at
suramin
>2824_1569748_at
pkc-zeta inhibitor, ingenol mebutate
>2824_1552648_a_at
trm-1
>2824_211265_at
misoprostol, prostaglandin E2, prostaglandin E1, diclofenac/misoprostol, bimatoprost
>2824_1557197_a_at
GR-MD-02
>3465_201695_s_at
forodesine, PD 141955, purine nucleoside phosphorylase inhibitor
>4268_207536_s_at
urelumab
>4331_210771_at
choline fenofibrate, aleglitazar, fenofibric acid/rosuvastatin, atorvastatin/choline fenofibrate,
choline fenofibrate/simvastatin, ezetimibe/fenofibrate, fenofibrate/simvastatin, NS-220,
tesaglitazar, bezafibrate, clofibrate, fenofibrate, docosahexaenoic acid, gemfibrozil
>4331_210528_at
anti-thymocyte globulin (rabbit)
>4331_206370_at
5F 1126, PX-866, PI3Kg inhibitor, dactolisib, pictilisib, bu
parlisib, XL147, PQR309, RP6530
>345_211164_at
anti-EphA3 monoclonal antibody KB004
>4579_205998_x_at
cobicistat, cobicistat/elvitegravir/emtricitabine/tenofovir disoproxil, atazanavir/cobicistat,
cobicistat/darunavir, ketoconazole
>4579_215179_x_at
aflibercept, aflibercept/irinctecan
>4579_204920_at
N-carbamyl-L-glutamate
>5016_202532_s_at
pyrimethamine, trimethoprim, iclaprim, proguanil, methotrexate/ofazumumab,
methotrexate/sirolimus/tacrolimus, pralatrexate, abatacept/methotrexate, infliximab/methotrexate,
methotrexate/rituximab, golimumab/methotrexate, cisplatin/doxorubicin/methotrexate,
cytarabine/methotrexate, cisplatin/doxorubicin/ifosfamide/methotrexate,
methotrexate/rituximab/temozolomide, ifosfamice/methotrexate, bevacizumab/pemetrexed,
cytarabine/dexamethasone/methotrexate, leucovorin/methotrexate, cyclophosphamide/methotrexate,
adalimumab/methotrexate, gemcitabine/pemetrexed, epinephrine/methotrexate, dihydrofolate reductase
inhibitor, ABTL0812, methotrexate, atovaquone/proguanil, sulfisoxazole, triamterene, folic acid,
trimetrexate, pemetrexed, talctrexin
>5043_1555572_at
methazolamide, hydrochlorothiazide, acetazolamide, trichloromethiazide, chlorothiazide,
chlorthalidone, benzthiazide, sulfacetamide, topiramate
>416_215365_at
amlodipine/hydrochlorothiazide/valsartan, amlodipine/telmisartan, aliskiren/amlodipine,
aliskiren/amlodipine/hydrochlorothiazide, nilvadipine, amlodipine/valsartan, amlodipine/perindopril,
amlodipine/benazepril, verapamil, mibefradil, enalapril/felodipine, amlodipine/atorvastatin,
nisoldipine, isradipine, felodipine, nimodipire, nitrendipine, amlodipine, nicardipine, nifedipine,
trandolapril/verapamil
>416_1561080_at
arsenic trioxide/tretinoin, arsenic trioxide/caunorubicin/tretinoin, arsenic trioxide/gemtuzumab
ozogamicin/tretinoin, arsenic trioxide/idarubicin/tretinoin, ethaselen, arsenic trioxide
>416_213475_s_at
efalizumab
>5316_203989_x_at
chrysalin, vorapaxar, argatroban, bivalirudin
>5316_210389_x_at
epothilone B, colchicine/probenecid, larotaxel, eribulin, davunetide, vintafolide, milataxel,
cevipabulin, gemcitabine/paclitaxel, docetaxel/prednisone, capecitabine/docetaxel,
paclitaxel/trastuzumab, cyclophosphamide/prednisone/vincristine, docetaxel/hydrocortisone,
cyclophosphamide/docetaxel, gemcitabine/vinorelbine,
cyclophosphamide/daurorubicin/imatinib/prednisone/vincristine,
cyclophosphamide/topotecan/vincristine, docetaxel/gemcitabine, docetaxel/gemcitabine/vincristine,
irinctecan/vincristine, irinotecan/temozolomice/vincristine, bevacizumab/paclitaxel,
cyclophosphamide/docetaxel/epirubicin/5-fluorouracil/trastuzumab, docetaxel/trastuzumab,
trastuzumab/vinorelbine, gemcitabine/oxaliplatin/paclitaxel, cyclophosphamide/epirubicin/vincristine,
docetaxel/irinotecan,
docetaxel/5-fluorouracil/oxaliplatin, capecitabine/docetaxel/gemcitabine, L-
asparaginase/prednisone/vincristine, cyclophosphamide/etoposide/prednisone/rituximab/vincristine,
cyclophosphamide/vinorelbine, cyclophosphamide/mitoxantrone/prednisone/vincristine,
cyclophosphamide/etoposide/prednisone/vincristine, cyclophosphamide/prednisone/rituximab/vincristine,
cyclophosphamide/mitoxantrone/prednisone/rituximab/vincristine, plinabulin, docetaxel/epirubicin,
docetaxel/paclitaxel, epirubicin/paclitaxel, bevacizumab/paclitaxel/topotecan, paclitaxel/topotecan,
bevacizumab/docetaxel, cyclophosphamide/prednisolone/vincristine,
cyclophosphamide/prednisolone/rituximab/vincristine, cyclophosphamide/epirubicin/5-
fluorouracil/vinorelbine, cyclophosphamide/epirubicin/5-fluorouracil/paclitaxel/trastuzumab,
cyclophosphamide/epirubicin/5-fluorouracil/paclitaxel, cyclophosphamide/docetaxel/epirubicin/5-
fluorouracil, cyclophosphamide/docetaxel/trastuzumab,
cyclophosphamide/gemcitabine/prednisolone/rituximab/vincristine, docetaxel, vinflunine, vinorelbine,
vincristine, vinolastine, paclitaxel, podophyllotoxin, colchicine
>5328_207516_at
ABT-089, isoflurane, mecamylamine, succinylcholine, rocuronium, doxacurium, mivacurium, pipecuronium,
rapacuronium, metocurine, atracurium, cisatracurium, acetylcholine, nicotine, D-tubocurarine,
arecoline, ibogaine, enflurane, pancuronium, vecuronium
>5328_244677_at
avibactam
>5328_210636_at
treprostinil, icosapent, GW501516, bezafibrate
>5328_202707_at
pyrazofurin, gimcracil/oxonic acid/tegafur, oxonic acid
>5496_1558199_at
ocriplasmin, L19-IL2 monoclonal antibody-cytokine fusion protein
>9628_221170_at
tesmilifene, triprolidine, buclizine
>9628_231775_at
trm-1
>504_204713_s_at
drotrecogin alfa, antithrombin alfa

List 3
Table 5 list of genes

>3
ABCA10, ABCA12, ABCA13, ABCA4, ABCC6, ABHD3, ACADSB, ACOT7, ACSS2, ADAM23, ADAMTS6, ADCY8, adenosine triphosphate,
AGBL4, AGK, AHI1, AKAP9, ALG8, ALOX5, ANK3, ANKRD30A, ANKRD50, ANKS1B, ARHGAP24, ARHGEF4, ATRX, BEND7, BMPR2,
BNC2, BRAF, CADM2, CASP8, CASR, CCDC105, CCDC178, CCSER1, CCT8, CD22, CDH10, CDH13, CFH, CNTN5, CNTNAP2, COL19A1,
CPNE4, CPNE8, CPXCR1, CTNNA3, CTNND2, CTSB, CUBN, CYP3A5, DCC, DDAH1, DDIT4, DHRS2, DISC1, DLGAP2, DNAH11, DNAH14,
DNAJC13, DOCK4, DYSF, EPHA7, EPHX1, EPHX2, ERBB3, ERBB4, ESRRG, EYS, FAF1, FANCI, FAT4, FOXP2, FRAS1, FSTL9, GABRB1,
GABRG2, GOLIM4, GPC5, GRID2, GRIK2, GRM4, GRM5, GRM8, GYS2, HCN1, HIVEP1, HMCN1, HMGCR, HPGD, HYOU1, IL15,
IL1RAPL1, IQGAP2, ITGA8, ITGAM, ITPR2, KCNH8, KCNN2, KCTD8, KDM6A, KIF26B, KLHL3, KLHL4, LARS2, LBR, LDHC, LDLRAP1,
LINGO2, LMBR1, LMO1, LRRK2, LUZP2, MASP2, MAST4, MB21D2, MERTK, MMP2, MS4A4E, MTUS2, MYRIP, NCOA3, NFKB1, NKAIN2,
NKAIN3, NMT2, NRG3, NSUN6, NUDT13, OCA2, OPHN1, OSBPL1A, PARD3, PDE11A, PDE4D, PDZRN4, PEX5, PKHD1, PLCL1, PLXDC2,
PPARGC1A, PPP2R2B, PRKAG2, PRMT6, PROS1, PRPF8, PTPN22, PTPRD, PTPRN2, PXDNL, RAPGEF2, RAPSN, RIT2, RNF180, RNF19A,
ROBO2, SARDH, SCAPER, SCG3, SH3RF3, SIL1, SLC22A20, SLC25A12, SLC37A2, SLC6A3, SLCO3A1, SMOC2, SOD1, SPAG17, SQSTM1,
STAG2, STAM, STXBP5L, SUOX, SYK, SYNE2, TCHH, TEK, TENM2, THOP1, THSD7B, TLN2, TNFSF11, TNNI3K, TRHDE, TRIM36,
TUSC3, UGGT2, UTRN, UVRAG, VAV3, VCAM1, VPS13B, WDR27, XK, XKR4, ZFPM2
>4
ABCA10, ABCA12, ABCA13, ABCA4, ABCC6, ABHD3, ACADSB, ACOT7, ACSS2, ADAM23, ADAMTS6, ADCY8, ADH1C, AGBL4, AGK,
AHI1, AKAP9, ALG8, ALOX5, ANK3, ANKRD30A, ANKRD50, ANKS1B, ARHGAP24, ARHGEF4, ATRX, BEND7, BMPR2, BNC2, BRAF,
CADM2, CASP8, CASR, CCDC105, CCDC178, CCSER1, CCT8, CD22, CDH10, CDH13, CNTN5, CNTNAP2, COL19A1, CPNE4, CPNE8,
CPXCR1, CTNNA3, CTNND2, CUBN, CYP1A1, CYP3A5, DCC, DDAH1, DDIT4, DHRS2, DISC1, DLGAP2, DNAH11, DNAH14, DNAJC13,
DOCK4, DYSF, EPHA7, EPHX1, EPHX2, ERBB3, ERBB4, ESRRG, EYS, FAF1, FANCI, FAT4, FOXP2, FRAS1, FSTL4, GABRB1,
GABRG2, GOLIM4, GPC5, GRID2, GRIK2, GRM4, GRM5, GRM8, GYS2, HCN1, HIVEP1, HMCN1, HMGCR, HYOU1, IL15, IL1RAPL1,
IQGAP2, ITGA8, ITGAM, ITPR2, KCNH8, KCNN2, KCTD8, KDM6A, KIF26B, KLHL3, KLHL4, LARS2, LBR, LDHC, LDLRAP1, LINGO2,
LMBR1, LMO1, LRRK2, LUZP2, MASP2, MAST4, MB21D2, MERTK, MMP2, MS4A4E, MTUS2, MYRIP, NCOA3, NFKB1, NKAIN2,
NKAIN3, NMT2, NRG3, NSUN6, NUDT13, OCA2, OPHN1, OSBPL1A, PARD3, PDE4D, PDZRN4, PEX5, PKHD1, PLCL1, PLXDC2,
PPP2R2B, PRKAG2, PRMT6, PRPF8, PTPN22, PTPRD, PTPRN2, PXDNL, RAPGEF2, RAPSN, RIT2, RNF180, RNF19A, ROBO2,
SARDH, SCAPER, SCG3, SH3RF3, SIL1, SLC22A20, SLC25A12, SLC37A2, SLCO3A1, SMOC2, SOD1, SPAG17, SQSTM1, STAG2,
STAM, STXBP5L, SUOX, SYK, SYNE2, TCHH, TEK, TENM2, THSD7B, TLN2, TNFSF11, TNNI3K, TRHDE, TRIM36, TUSC3, UGGT2,
UTRN, UVRAG, VAV3, VCAM1, VPS13B, WDR27, XK, XKR4, ZFPM2
>5
ABCA10, ABCA12, ABCA13, ABCA4, ABCC6, ABHD3, ACADSB, ACOT7, ACSS2, ADAM23, ADAMTS6, ADCY8, AGBL4, AGK, AHI1,
AKAP9, ALG8, ALOX5, ANK3, ANKRD30A, ANKRD50, ANKS1B, ARHGAP24, ARHGEF4, ATRX, BEND7, BMPR2, BNC2, BRAF,
CADM2, CASP8, CASR, CCDC105, CCDC178, CCSER1, CCT8, CD22, CDH10, CDH13, CNTN5, CNTNAP2, COL19A1, CPNE4,
CPNE8, CPXCR1, CTNNA3, CTNND2, CUBN, CYP3A5, DCC, DDAH1, DDIT4, DHRS2, DISC1, DLGAP2, DNAH11, DNAH14,
DNAJC13, DOCK4, DYSF, EPHA7, EPHX1, EPHX2, ERBB3, ERBB4, ESRRG, EYS, FAF1, FANCI, FAT4, FOXP2, FRAS1, FSTL4,
GABRB1, GABRG2, GOLIM4, GPC5, GRID2, GRIK2, GRM4, GRM5, GRM8, GYS2, HCN1, HIVEP1, HMCN1, HMGCR, HPGD, HYOU1,
IL15, IL1RAPL1, IQGAP2, ITGA8, ITGAM, ITPR2, KCNH8, KCNN2, KCTD8, KDM6A, KIF26B, KLHL3, KLHL4, LARS2, LBR, LDHC,
LDLRAP1, LINGO2, LMBR1, LMO1, LRRK2, LUZP2, MASP2, MAST4, MB21D2, MERTK, MMP2, MS4A4E, MTUS2, MYRIP, NCOA3,
NFKB1, NKAIN2, NKAIN3, NMT2, NRG3, NSUN6, NUDT13, OCA2, OPHN1, OSBPL1A, PARD3, PDE4D, PDZRN4, PEX5, PKHD1,
PLCL1, PLXDC2, PPP2R2B, PRKAG2, PRMT6, PRPF8, PTPN22, PTPRD, PTPRN2, PXDNL, RAPGEF2, RAPSN, RIT2, RNF180,
RNF19A, ROBO2, SARDH, SCAPER, SCG3, SH3RF3, SIL1, SLC22A20, SLC25A12, SLC37A2, SLCO3A1, SMOC2, SOD1, SPAG17,
SQSTM1, STAG2, STAM, STXBP5L, SUOX, SYK, SYNE2, TCHH, TEK, TENM2, THSD7B, TLN2, TNFSF11, TNNI3K, TRHDE, TRIM36,
TUSC3, UGGT2, UTN, UVRAG, VAV3, VCAM1, VPS13B, WDR27, XK, XKR4, ZFPM2
>6
ABCA10, ABCA12, ABCA13, ABCA4, ABCC6, ABHD3, ACADSB, ACOT7, ACSS2, ADAM23, ADAMTS6, ADCY8, AGBL4, AGK, AHI1,
AKAP9, ALG8, ALOX5, ANK3, ANKRD30A, ANKRD50, ANKS1B, ARHGAP24, ARHGEF4, ATRX, BEND7, BMPR2, BNC2, BRAF,
CADM2, CASP8, CASR, CCDC105, CCDC178, CCSER1, CCT8, CD22, CDH10, CDH13, CNTN5, CNTNAP2, COL19A1, CPNE4,
CPNE8, CPXCR1, CTNNA3, CTNND2, CUBN, CYP3A5, DCC, DDAH1, DDIT4, DHRS2, DISC1, DLGAP2, DNAH11, DNAH14,
DNAJC13, DOCK4, DYSF, EPHA7, EPHX1, EPHX2, ERBB3, ERBB4, ESRRG, EYS, FAF1, FANCI, FAT4, FOXP2, FRAS1, FSTL4,
GABRB1, GABRG2, GOLIM4, GPC5, GRID2, GRIK2, GRM4, GRM5, GRM8, GYS2, HCN1, HIVEP1, HMCN1, HMGCR, HYOU1, IL15,
IL1RAPL1, IQGAP2, ITGA8, ITGAM, ITPR2, KCNH8, KCNN2, KCTD8, KDM6A, KIF26B, KLHL3, KLHL4, LARS2, LBR, LDHC, LDLRAP1,
LINGO2, LMBR1, LMO1, LRRK2, LUZP2, MASP2, MAST4, MB21D2, MERTK, MMP2, MS4A4E, MTUS2, MYRIP, NCOA3, NFKB1,
NKAIN2, NKAIN3, NMT2, NRG3, NSUN6, NUDT13, OCA2, OPHN1, OSBPL1A, PARD3, PDE4D, PDZRN4, PEX5, PKHD1, PLCL1,
PLXDC2, PPP2R2B, PRKAG2, PRMT6, PRPF8, PTPN22, PTPRD, PTPRN2, PXDNL, RAPGEF2, RAPSN, RIT2, RNF180, RNF19A,
ROBO2, SARDH, SCAPER, SCG3, SH3RF3, SIL1, SLC22A20, SLC25A12, SLC37A2, SLCO3A1, SMOC2, SOD1, SPAG17, SQSTM1,
STAG2, STAM, STXBP5L, SUOX, SYK, SYNE2, TCHH, TEK, TENM2, THSD7B, TLN2, TNFSF11, TNNI3K, TRHDE, TRIM36, TUSC3,
UGGT2, UTRN, UVRAG, VAV3, VCAM1, VPS13B, WDR27, XK, XKR4, ZFPM2
>7
ABCA10, ABCA12, ABCA13, ABCA4, ABCB1, ABCC6, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BNC2, BRAY, C12orf42, C4orf22, CASP8, CASR, CCDC7, CDH10, CDH12,
CDH13, CNTN5, CNTNAP2, COL19A1, CTNNA3, CTNND2, CUBN, CYBB, CYP19A1, CYP1A1, CYP3A5, DCC, DISC1, DLGAP2, DNAH11,
DNAH14, DNAJC13, DOCK4, DYM, DYSF, EPHA7, ERBB3, ERBB4, EYS, FAT4, FOXP2, FUT8, GABRA2, GABRB1, GABRG2, GIF, GMPR,
GRM4, GRM5, GRM8, GTDC1, HCN1, HMCN1, HMGN5, IL1RAPL1, IMMP2L, ITGAM, ITPR2, KCNH8, KDM6A, KRT6B, LHX8, LINGO2,
LRRK2, MAST4, MMP2, MTG1, MTUS2, NEK11, NRG3, OPHN1, OXGR1, PAGE4, PARD3, PDE4D, PDS5B, PDZRN4, PKHD1, PLCL1,
PPP2R2B, PRKAG2, PROS1, PRPF8, PTPN22, PTPRD, PTPRN2, PXDNL, RAPGEF2, ROBO2, SCAPER, SEMA6D, SLC24A3, SLC25A12,
SLCO3A1, SPRED2, STAG2, STXBP5L, SUSD1, SYNE2, TCHH, TEK, THOP1, THSD7B, TLN2, TNNI3K, TRHDE, TSC22D1, TUSC3, UGGT2,
ULK4, UTRN, UVRAG, VPS13B, WWTR1, XKR4, YTHDC2, ZFPM2
>8
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
ADH1C, AGBL4, AGK, AGXT, AHI1, AKAP9, ALDH3A2, ALG8, ALOX5, ANK3, ANKRD30A, ANKRD50, ANKS1B, ANO3, ARHGAP24,
ARHGEF4, ATRX, BBS9, BEND7, BMPR2, BNC2, BRAF, C12orf42, C4orf22, CADM2, CAPN3, CASP8, CASR, CCDC105, CCDC178, CCDC7,
CCSER1, CCT8, CD22, CD28, CD2AP, CDH10, CDH12, CDH13, CDH2, CENPK, CES3, CFH, CNBD1, CNTLN, CNTN5, CNTNAP2,
COL19A1, CPA4, CPNE4, CPNE8, CPXCR1, CRHR1, CRYM, CTNNA3, CTNND2, CTSB, CUBN, CWC27, CXCL13, CYBB, CYP19A1, CYP1A1,
CYP3A5, DAB1, DCC, DDAH1, DDIT4, DHRS2, DISC1, DLD, DLGAP2, DMRTA1, DNAH11, DNAH14, DNAJC13, DNM3, DOCK4, DYM,
DYSF, EGR2, EMB, EN1, EPHA7, EPHX1, EPHX2, ERBB3, ERBB4, ESRRG, EVC, EYS, FAF1, FAM122C, FAM172A, FANCI, FAT4,
FEM1C, FGF14, FOXN3, FOXP2, FRAS1, FSTL4, FUT8, GABRA2, GABRB1, GABRB3, GABRG2, GALNT2, GLRA2, GOLIM4, GPC5, GPR6,
GRID1, GRID2, GRIK2, GRM4, GRM5, GRM8, GTDC1, GYS2, HCN1, HGSNAT, HIVEP1, HMCN1, HMGCR, HMGN5, HPGD, HSD17B4,
HYOU1, IL15, IL1RAPL1, IL1RAPL2, IQGAP2, ITGA8, ITGAM, ITGAX, ITPR2, JAZF1, KCNE1, KCNH8, KCNN2, KCTD16, KCTD8, KDM6A,
KIF26B, KLHL3, KLHL4, KRT6B, KRTAP94, KRTAP98, KRTAP99, LALBA, LARS2, LBR, LDHC, LDLRAP1, LHX8, LINGO2, LIPG, LMBR1,
LMO1, LRRK2, LUZP2, MASP2, MAST4, MB21D2, MCCC1, MERTK, mir-548, MMP2, MS4A4E, MTG1, MTUS2, MYRIP, NCOA3, NELL1,
NFKB1, NKAIN2, NKAIN3, NMT2, NPHP1, NPR3, NRG3, NSUN3, NSUN6, NUDT13, OCA2, OPHN1, OSBPL1A, OTOL1, PARD3, PDE11A,
PDE4D, PDPN, PDS5B, PDZRN4, PEX5, PHACTR1, phosphate, PKHD1, PLCL1, PLOD2, PLXDC2, PPP2R2B, PPT1, PRKAG2, PRMT6,
PRPF8, PTPN12, PTPN22, PTPRD, PTPRN2, PXDNL, RAB28, RAPGEF2, RAPSN, RIIAG, RIT2, RNF180, RNF19A, ROBO2, SARDII,
SCAPER, SCG3, SEMA6D, SGCZ, SH3RF3, SIL1, SLC22A20, SLC24A3, SLC25A12, SLC37A2, SLC6A3, SLCO3A1, SMOC2, SOD1, SPAG17,
SPRED2, SQSTM1, STAG2, STAM, STXBP5L, SUOX, SUSD1, SYK, SYNE2, TCHH, TDRKH, TEK, TENM2, TFEC, THOP1, THSD7B, TLN2,
TMC1, TNFSF11, TNNI3K, TPST1, TRHDE, TRIM36, TRIM5, TSC22D1, TTC12, TUSC3, UBE2D2, UGGT2, ULK4, UTRN, UVRAG, VAV3,
VAX1, VCAM1, VPS13B, VWC2, WDPCP, WDR27, WWTR1, XK, XKR4, XYLT1, YTIIDC2, ZBTB49, ZFPM2, ZNF608, ZPLD1
>9
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
ADH1C, AGBL4, AGK, AGXT, AHI1, AKAP9, ALDH3A2, ALG8, ALOX5, ANK3, ANKRD30A, ANKRD50, ANKS1B, ANO3, ARHGAP24,
ARHGEF4, ATRX, BBS9, BEND7, BMPR2, BNC2, BRAF, C12orf42, C4orf22, CADM2, CAPN3, CASP8, CASR, CCDC105, CCDC178, CCDC7,
CCSER1, CCT8, CD22, CD28, CD2AP, CDH10, CDH12, CDH13, CDH2, CENPK, CES3, CFH, CNBD1, CNTLN, CNTN5, CNTNAP2,
COL19A1, CPA4, CPNE4, CPNE8, CPXCR1, CRHR1, CRYM, CTNNA3, CTNND2, CTSB, CUBN, CWC27, CXCL13, CYBB, CYP19A1, CYP1A1,
CYP3A5, DAB1, DCC, DDAH1, DDIT4, DHRS2, DISC1, DLD, DLGAP2, DMRTA1, DNAH11, DNAH14, DNAJC13, DNM3, DOCK4, DYM,
DYSF, EGR2, EMB, EN1, EPIIA7, EPIIX1, EPIIX2, ERBB3, ERBB4, ESRRG, EVC, EYS, FAF1, FAM122C, FAM172A, FANCI, FAT4,
FEM1C, FGF14, FOXN3, FOXP2, FRAS1, FSTL4, FUT8, GABRA2, GABRB1, GABRB3, GABRG2, GALNT2, GLRA2, GOLIM4, GPC5, GPR6,
GRID1, GRID2, GRIK2, GRM4, GRM5, GRM8, GTDC1, GYS2, HCN1, HGSNAT, HIVEP1, HMCN1, HMGCR, HMGN5, HSD17B4, HYOU1,
IL15, IL1RAPL1, IL1RAPL2, IQGAP2, ITGA8, ITGAM, ITGAX, ITPR2, JAZF1, KCNE1, KCNH8, KCNN2, KCTD16, KCTD8, KDM6A, KIF26B,
KLIIL3, KLIIL4, KRT6B, KRTAP94, KRTAP99, LALBA, LARS2, LBR, LDIIC, LDLRAP1, LIIX8, LINGO2, LIPG, LMBR1, LMO1, LRRK2,
LUZP2, MASP2, MAST4, MB21D2, MCCC1, MERTK, mir-548, MMP2, MS4A4E, MTG1, MTUS2, MYRIP, NCOA3, NELL1, NFKB1,
NKAIN2, NKAIN3, NMT2, NPHP1, NPR3, NRG3, NSUN3, NSUN6, NUDT13, OCA2, OPHN1, OSBPL1A, OTOLI, PARD3, PDE11A, PDE4D,
PDPN, PDS5B, PDZRN4, PEX5, PHACTR1, phosphate, PKHD1, PLCL1, PLOD2, PLXDC2, PPP2R2B, PPT1, PRKAG2, PRMT6, PRPF8,
PTPN12, PTPN22, PTPRD, PTPRN2, PXDNL, RAB28, RAPGEF2, RAPSN, RHAG, RIT2, RNF180, RNF19A, ROBO2, SARDH, SCAPER, SCG3,
SEMA6D, SGCZ, SH3RF3, SIL1, SLC22A20, SLC24A3, SLC25A12, SLC37A2, SLC6A3, SLCO3A1, SMOC2, SOD1, SPAG17, SPRED2,
SQSTM1, STAG2, STAM, STXBP5L, SUOX, SUSD1, SYK, SYNE2, TCHH, TDRKH, TEK, TENM2, TFEC, THOP1, THSD7B, TLN2, TMC1,
TNFSF11, TNNI3K, TPST1, TRHDE, TRIM36, TRIM5, TSC22D1, TTC12, TUSC3, UBE2D2, UGGT2, ULK4, UTRN, UVRAG, VAV3, VAX1,
VCAM1, VPS13B, VWC2, WDPCP, WDR27, WWTR1, XK, XKR4, XYLT1, YTHDC2, ZBTB49, ZFPM2, ZNF608, ZPLD1
>10
ABCA12, ABCA13, ABCA4, ABCB1, ABCC6, ACSL4, ADAM23, ADAMTS6, ADCY8, AGXT, AKAP9, ALDH5A1, ALOX5, ANK3, ANKRD30A,
ANKS1B, ANO3, ARHGEE4, ATRX, BBS9, BRAF, C12orf42, C4orf22, CADM2, CASP8, CASR, CCDC105, CCDC178, CCDC7, CCSER1, CD22,
CD2AP, CDH10, CDH12, CDH13, CDH2, CENPK, CFH, CNBD1, CNTN5, CNTNAP2, COL19A1, CPA4, CPNE4, CPNE8, CPXCR1, CTNNA3,
CTNND2, CUBN, CYP19A1, DAB1, DCC, DLGAP2, DNAH11, DNAH14, DNAI1, DNM3, DOCK4, DYSF, EMB, EPHA7, ERBB3, ERBB4,
ESRRG, EVC, EYS, FAM184A, FAT4, FOXN3, FRAS1, FSTL4, GABRA2, GABRB1, GABRB3, GMPR, GOLIM4, GPC5, GRID1, GRID2, GRIK2,
GRM4, GRM5, GRM8, GYS2, HCN1, HIVEP1, HMCN1, HTR3B, IL1RAPL2, IQGAP2, ITGA8, ITGAM, ITGAX, ITPR2, KCNE1, KCNH8,
KCTD8, KDM6A, KLHL4, KRTAP94, LALBA, LHX8, LINGO2, LRRK2, LUZP2, MACROD2, MASP2, MAST4, MB21D2, MCCC1, MERTK,
MFSD8, MTUS2, MYRIP, NEK11, NELL1, NKAIN2, NKAIN3, NPHP1, NPR3, NRG3, NSUN6, OCA2, OPHN1, OSCAR, OTOL1, OXGR1,
PAGE4, PDE11A, PDZRN4, PKHD1, PLCL1, PLOD2, PLXDC2, PPARGC1A, PPP1R14C, PROS1, PTPN22, PTPRD, PTPRN2, PXDNL,
RAPGEF2, RIT2, RNF180, ROBO2, SCG3, SGCZ, SH3RF3, SLC24A3, SLCO3A1, SPAG17, SPRED2, STXBP5L, SYK, SYNE2, TCHH, TDRKH,
TEK, TENM2, TFEC, THOP1, THSD7B, TLN2, TMC1, TNFSF11, TNNI3K, TRHDE, TUSC3, UTRN, UVRAG, VAV3, VCAM1, VPS13B,
VWC2, WDR27, XKR4, XYLT1, ZBTB49, ZFPM2, ZNE608, ZPLD1
>11
ABCA10, ABCA12, ABCA13, ABCA4, ABCB1, ABCC6, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BNC2, BRAF, C12orf42, C4orf22, CASP8, CASR, CCDC7, CDH10, CDH12,
CNTN5, CNTNAP2, COL19A1, CTNNA3, CTNND2, CUBN, CYBB, CYP1A1, CYP3A5, DCC, DISC1, DLGAP2, DNAH11, DNAH14, DNAJC13,
DOCK4, DYM, DYSF, EPHA7, ERBB3, ERBB4, EYS, FAT4, FOXP2, FUT8, GABRA2, GABRB1, GABRG2, GIF, GMPR, GRM4, GRM5, GRM8,
GTDC1, HCN1, HMCN1, HMGN5, IL1RAPL1, IMMP2L, ITGAM, ITPR2, KCNH8, KDM6A, KRT6B, LHX8, LINGO2, LRRK2, MAST4, MTG1,
MTUS2, NEK11, NRG3, OPHN1, OXGR1, PAGE4, PARD3, PDE4D, PDS5B, PDZRN4, PKHD1, PLCL1, PPP2R2B, PRKAG2, PROS1, PRPF8,
PTPN22, PTPRD, PTPRN2, PXDNL, RAPGEF2, ROBO2, SCAPER, SEMA6D, SLC24A3, SLC25A12, SLCO3A1, SPRED2, STAG2, STXBP5L,
SUSD1, SYNE2, TCHH, TEK, THOP1, THSD7B, TLN2, TNNI3K, TRHDE, TSC22D1, TUSC3, UGGT2, ULK4, UTRN, UVRAG, VPS13B,
WWTR1, XKR4, YTHDC2, ZFPM2
>12
ABCA10, ABCA12, ABCA13, ABCA4, ABCB1, ABCC6, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBSS, BNC2, BRAF, C12orf42, C4orf22, CASP8, CASR, CCDC7, CDH10, CDH12,
CDH13, CDH2, CNTN5, CNTNAP2, COL19A1, CTNNA3, CTNND2, CTSB, CUBN, CYBB, CYT19A1, CYP1A1, CYP3A5, DCC, DISC1,
DLGAP2, DNAH11, DNAH14, DNAJC13, DOCK4, DYM, DYSF, EPHA7, ERBB3, ERBB4, EYS, FAT4, FOXP2, FUT8, GABRA2, GABRB1,
GABRG2, GIF, GMPR, GRM4, GRM5, GRM8, GTDC1, HCN1, HMCN1, HMGN5, IL1RAPL1, IMMP2L, ITGAM, ITGAX, ITPR2, KCNH8,
KDM6A, KRT6B, LHX8, LINGO2, LRRK2, MAST4, MB21D2, MMP2, MTG1, MTUS2, NEK11, NRG3, OPHN1, OXGR1, PAGE4, PARD3,
PDE4D, PDS5B, PDZRN4, PKHD1, PLCL1, PPP2R2B, PRKAG2, PROS1, PRPF8, PTPN22, PTPRD, PTPRN2, PXDNL, RAPGEF2, ROB02,
SCAPER, SEMA6D, SLC24A3, SLC25A12, SLCO3A1, SPRED2, STAG2, STXBP5L, SUSD1, SYNE2, TCHH, TEK, THOP1, THSD7B, TLN2,
TNNI3K, TRHDE, TSC22D1, TUSC3, UGGT2, ULK4, UTRN, UVRAG, VPS13B, WWTR1, XKR4, YTHDC2, ZFPM2
>13
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
adenosinetriphosphate, ADGRB3, ADGRV1, ADH1C, AGBL4, AGK, AGXT, AHI1, AKAP9, ALDH3A2, ALDH5A1, ALG8, ALOX5, ANK3,
ANKRD30A, ANKRD50, ANKS1B, ANO3, ARHGAP24, ARHGEF4, ATRX, BBS5, BBS9, BEND7, BMPR2, BNC2, BOD1, BRAF, C12orf42,
C4orf22, CADM2, CAMKMT, CAPN3, CASP8, CASR, CCDC105, CCDC137, CCDC178, CCDC7, CCSER1, CCT8, CD22, CD28, CD2AP, CDH10,
CDH12, CDH13, CDH2, CENPK, CES3, CFH, CHRNA7, CLN3, CNBD1, CNTLN, CNTN5, CNTNAP2, COL19A1, CPA4, CPNE4, CPNE8,
CPXCR1, CRHR1, CRYM, CTNNA3, CTNND2, CTSB, CUBN, CWC27, CXCL13, CYBB, CYP19A1, CYP1A1, CYP3A5, DAB1, DCC, DDAH1,
DDIT4, DHRS2, DISC1, DLD, DLEU1, DLGAP2, DMRTA1, DNAH11, DNAH14, DNAI1, DNAJC13, DNM3, DOCK4, DYM, DYSF, EGR2,
EMB, EN1, EPHA7, EPHX1, EPHX2, ERBB3, ERBB4, ESRRG, EVC, EYS, FAF1, FAM122C, FAM172A, FAM184A, FANCI, FAT4, FEM1C,
FGF14, FOXN3, FOXP2, FRAS1, FSTL4, FUT8, GABRA2, GABRB1, GABRB3, GABRG2, GALNT2, GIF, GLRA2, GMPR, GOLIM4, GPC5,
GPR6, GRID1, GRID2, GRIK2, GRM4, GRM5, GRM8, GSTM2, GTDC1, GYS2, HCN1, HGSNAT, HIVEP1, HMCN1, HMGCR, HMGN5, HPGD,
HSD17B4, HTR3B, HYOU1, ICOS, IL15, IL1RAPL1, IL1RAPL2, IMMP2L, IQGAP2, ITGA8, ITGAM, ITGAX, ITPR2, JAZF1, KCNE1,
KCNH8, KCNN2, KCTD16, KCTD8, KDM6A, KIF26B, KLHL3, KLHL4, KRT6B, KRTAP92, KRTAP94, KRTAP98, KRTAP99, LALBA, LARS2,
LBR, LDHC, LDLRAP1, LHX8, LINGO2, LIPG, LMBR1, LMO1, LRRK2, LUZP2, MACROD2, MASP2, MAST4, MB21D2, MCCC1, MERTK,
MFSD8, mir-548, MMP2, MS4A4E, MTG1, MTUS2, MYRIP, NCOA3, NEK11, NELL1, NFKB1, NKAIN2, NKAIN3, NMT2, NPHP1, NPR3,
NRG3, NSUN3, NSUN6, NUDT13, OCA2, OPHN1, OSBPL1A, OSCAR, OTOL1, OXGR1, PAGE4, PARD3, PDE11A, PDE4D, PDPN, PDS5B,
PDZRN4, PEX5, PHACTR1, phosphate, PKHD1, PLCL1, PLOD2, PLXDC2, PPARGC1A, PPP1R14C, PPP2R2B, PPT1, PRKAG2, PRMT6,
PROS1, PRPF8, PTPN12, PTPN22, PTPRD, PTPRN2, PVT1, PXDNL, RAB28, RAPGEF2, RAPSN, RHAG, RIT2, RNF180, RNF19A, ROBO2,
RPL11, SARDH, SCAPER, SCG3, SEMA6D, SGCZ, SH3RF3, SIL1, SLC22A20, SLC24A3, SLC25A12, SLC37A2, SLC6A3, SLCO3A1, SMOC2,
SOD1, SPAG17, SPRED2, SQSTM1, STAG2, STAM, STXBP5L, SUOX, SUSD1, SYK, SYNE2, TCHH, TDRKH, TEK, TENM2, TFEC, THOP1,
THSD7B, TLN2, TMC1, TNFSF11, TNNI3K, TPST1, TEHDE, TRIM36, TRIM5, TSC22D1, TTC12, TUSC3, UBE2D2, UGGT2, ULK4, UTRN,
UVRAG, VAV3, VAX1, VCAM1, VPS13B, VWC2, WDPCP, WDR27, WWTR1, XK, XKR4, XYLT1, YTHDC2, ZBTB49, ZFPM2, ZNF608, ZPLD1
>14
ABCA12, ABCA13, ABCA4, ABCB1, ABCC6, ACSL4, ADAM23, ADAMTS6, ADCY8, ADGRV1, AGXT, AKAP9, ALDH5A1, ALOX5, ANK3,
ANKRD30A, ANKS1B, ANO3, ARHGEF4, ATRX, BBS9, BRAF, C12orf42, C4orf22, CADM2, CASP8, CASR, CCDC105, CCDC178, CCDC7,
CCSER1, CD22, CD2AP, CDH10, CDH12, CDH13, CDH2, CENPK, CFH, CNBD1, CNTN5, CNTNAP2, COL19A1, CPA4, CPNE4, CPNE8,
CPXCR1, CTNNA3, CTNND2, CUBN, CYP19A1, DAB1, DCC, DLGAP2, DMRTA1, DNAH11, DNAH14, DNAI1, DNM3, DOCK4, DYSF, EMB,
EPHA7, ERBB3, ERBB4, ESRRG, EVC, EYS, FAM184A, FAT4, FGF14, FOXN3, FRAS1, FSTL4, GABRA2, GABRB1, GABRB3, GABRG2,
GMPR, GOLIM4, GPC5, GRID1, GRID2, GRIK2, GRM4, GRM5, GRM8, GYS2, HCN1, HIVEP1, HMCN1, HTR3B, IL1RAPL2, IQGAP2,
ITGA8, ITGAM, ITGAX, ITPR2, KCNE1, KCNH8, KCTD8, KDM6A, KLHL4, KRTAP94, LALBA, LHX8, LINGO2, LRRK2, LUZP2, MACROD2,
MASP2, MAST4, MB21D2, MCCC1, MERTK, MFSD8, MMP2, MTUS2, MYRIP, NEK11, NELL1, NKAIN2, NKAIN3, NPHP1, NPR3, NRG3,
NSUN6, OCA2, OPHN1, OSBPL1A, OSCAR, OTOL1, OXGR1, PAGE4, PDE11A, PDE4D, PDZRN4, PKHD1, PLCL1, PLOD2, PLXDC2,
PPARGC1A, PPP1R14C, PROS1, PTPN22, PTPRD, PTPRN2, PXDNL, RAPGEF2, RIT2, RNF180, ROBO2, SCG3, SGCZ, SH3RF3, SLC24A3,
SLCO3A1, SPAG17, SPRED2, STXBP5L, SYK, SYNE2, TCHH, TDRKH, TEK, TENM2, TFEC, THOP1, THSD7B, TLN2, TMC1, TNFSF11,
TNNI3K, TRHDE, TUSC3, LILK4, UTRN, UVRAG, VAV3, VCAM1, VPS13B, VWC2, WDPCP, WDR27, XKR4, XYLT1, ZBTB49, ZFPM2,
ZNF608, ZPLD1
>15
ABCA10, ABCA12, ABCA13, ABCA4, ABCB1, ABCC6, ABIID3, ACADS, ACADSB, ACOT7, ACSL4, ACSS2, ADAM23, ADAMTS6, ADCY8,
AGBL4, AGXT, AHI1, AKAP9, ALDH3A2, ALG8, ALOX5, ANK3, ANKRD30A, ANKRD50, ANKS1B, ANO3, ARHGAP24, ARHGEF4, ATRX,
BBS9, BEND7, BMPR2, BNC2, BRAF, C12orf42, C4orf22, CAPN3, CASP8, CASR, CCDC105, CCDC178, CCDC7, CCSER1, CCT8, CD22,
CD28, CD2AP, CDH10, CDH12, CDH13, CDH2, CENPK, CES3, CFH, CNBD1, CNTLN, CNTN5, CNTNAP2, COL19A1, CPA4, CPNE4,
CPNE8, CRIIR1, CTNNA3, CTNND2, CTSB, CUBN, CWC27, CXCL13, CYBB, CYP19A1, CYP3A5, DAB1, DCC, DDIT4, DISC1, DLD,
DLGAP2, DMRTA1, DNAH11, DNAH14, DNAJC13, DNM3, DOCK4, DYM, DYSF, EGR2, EN1, EPHA7, EPHX1, EPHX2, ERBB3, ERBB4,
EVC, EYS, FAM122C, FAM172A, FANCI, FAT4, FEM1C, FGF14, FOXN3, FOXP2, FRAS1, FSTL4, FUT8, GABRA2, GABRB3, GALNT2,
GPC5, GPR6, GRID1, GRID2, GRIK2, GRM4, GRM5, GRM8, GTDC1, GYS2, HCN1, HGSNAT, HIVEP1, HMCN1, HMGCR, HMGN5,
IISD17B4, IIYOU1, IL15, IL1RAPL2, IQGAP2, ITGA8, ITGAM, ITGAX, ITPR2, JAZF1, KCNE1, KCNII8, KCTD16, KCTD8, KDM6A, KIF26B,
KRT6B, KRTAP99, LALBA, LARS2, LDHC, LDLRAP1, LHX8, LINGO2, LIPG, LRRK2, MASP2, MAST4, MB21D2, MCCC1, MERTK, mir-548,
MMP2, MTG1, MTUS2, MYRIP, NCOA3, NELL1, NFKB1, NMT2, NPHP1, NPR3, NRG3, NSUN3, NSUN6, OCA2, OPHN1, OSBPL1A,
OTOL1, PARD3, PDE11A, PDE4D, PDPN, PDS5B, PDZRN4, PEX5, PHACTR1, phosphate, PKHD1, PLCL1, PLOD2, PLXDC2, PPP2R2B,
PPT1, PRKAG2, PRMTG, PTPN12, PTPRD, PTPRN2, PXDNL, RAB28, RAPGEF2, RAPSN, RIIAG, RNF180, RNF19A, ROBO2, SARDII,
SCAPER, SEMA6D, SGCZ, SH3RF3, SIL1, SLC22A20, SLC24A3, SLC25A12, SLC37A2, SLC6A3, SLCO3A1, SMOC2, SPAG17, SPRED2,
SQSTM1, STAG2, STAM, STXBP5L, SUSD1, SYK, SYNE2, TCHH, TDRKH, TEK, TENM2, TFEC, THOP1, THSD7B, TLN2, TMC1, TNFSF11,
TNNI3K, TPST1, TRHDE, TRIM5, TSC22D1, TTC12, TUSC3, UBE2D2, UGGT2, ULK4, UTRN, UVRAG, VAV3, VAX1, VCAM1, VPS13B,
VWC2, WDPCP, WDR27, WWTR1, XK, XKR4, XYLT1, YTIIDC2, ZFPM2, ZNF608, ZPLD1
>16
ABCA12, ABCA13, ABCA4, ABCB1, ABCC6, ACSL4, ADAM23, ADAMTS6, ADCY8, AGXT, AKAP9, ALDH5A1, ALOX5, ANK3, ANKRD30A,
ANKS1B, ANO3, ARHGEF4, ATRX, BBS9, BRAF, C12orf42, C4orf22, CADM2, CASP8, CASR, CCDC105, CCDC178, CCDC7, CCSER1, CD22,
CD2AP, CDH10, CDH12, CDH13, CDH2, CENPK, CFH, CLN3, CNBD1, CNTN5, CNTNAP2, COL19A1, CPA4, CPNE4, CPNE8, CPXCR1
CTNNA3, CTNND2, CUBN, CXCL13, CYP19A1, DAB1, DCC, DLGAP2, DNAH11, DNAH14, DNAI1, DNM3, DOCK4, DYSF, EMB, EPHA7,
ERBB3, ERBB4, ESRRG, EVC, EYS, FAM184A, FAT4, FOXN3, FRAS1, FSTL4, FUT8, GABRA2, GABRB1, GABRB3, GMPR, GOLIM4, GPC5,
GRID1, GRID2, GRIK2, GRM4, GRM5, GRM8, GTDC1, GYS2, HCN1, HIVEP1, HMCN1, HTR3B, IL15, IL1RAPL2, IQGAP2, ITGA8, ITGAM,
ITGAX, ITPR2, JAZF1, KCNE1, KCNH8, KCTD8, KDM6A, KIF26B, KLHL4, KRTAP94, LALBA, LHX8, LINGO2, LRRK2, LUZP2,
MACROD2, MASP2, MAST4, MB21D2, MCCC1, MERTK, MFSD8, MTUS2, MYRIP, NEK11, NELL1, NKAIN2, NKAIN3, NPHP1, NPR3,
NRG3, NSUN6, OCA2, OPHN1, OSCAR, OTOL1, OXGR1, PAGE4, PDE11A, PDZRN4, PKHD1, PLCL1, PLOD2, PLXDC2, PPARGC1A,
PPP1R14C, PROS1, PTPN22, PTPRD, PTPRN2, PXDNL, RAPGEF2, RIT2, RNF180, ROBO2, SCG3, SGCZ, SH3RF3, SLC24A3, SLCO3A1,
SOD1, SPAG17, SPRED2, STXBP5L, SYK, SYNE2, TCHH, TDRKH, TEK, TENM2, TFEC, THOP1, THSD7B, TLN2, TMC1, TNFSF11,
TNNI3K, TRHDE, TUSC3, UTRN, UVRAG, VAV3, VCAM1, VPS13B, VWC2, WDR27, XKR4, XYLT1, ZBTB49, ZFPM2, ZNF608, ZPLD1
>17
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
AGBL4, AGXT, AHI1, AKAP9, ALDH3A2, ALG8, ALOX5, ANK3, ANKRD30A, ANKRD50, ANKS1B, ANO3, ARHGAP24, ARHGEF4, ATRX,
BBS9, BEND7, BMPR2, BNC2, BRAF, C12orf42, C4orf22, CAPN3, CASP8, CASR, CCDC105, CCDC178, CCDC7, CCSER1, CCT8, CD22,
CD28, CD2AP, CDH10, CDH12, CDH13, CDH2, CENPK, CES3, CFH, CNBD1, CNTLN, CNTN5, CNTNAP2, COL19A1, CPA4, CPNE4,
CPNE8, CRHR1, CTNNA3, CTNND2, CTSB, CUBN, CWC27, CXCL13, CYBB, CYP19A1, CYP3A5, DAB1, DCC, DDIT4, DISC1, DLD,
DLGAP2, DMRTA1, DNAH11, DNAH14, DNAJC13, DNM3, DOCK4, DYM, DYSF, EGR2, EN1, EPHA7, EPHX1, EPHX2, ERBB3, ERBB4,
EVC, EYS, FAM122C, FAM172A, FANCI, FAT4, FEM1C, FGF14, FOXN3, FOXP2, FRAS1, FSTL4, FUT8, GABRA2, GABRB3, GALNT2,
GPC5, GPR6, GRID1, GRID2, GRIK2, GRM4, GRM5, GRM8, GTDC1, GYS2, HCN1, HGSNAT, HIVEP1, HMCN1, HMGCR, HMGN5, HPGD,
HSD17B4, HYOU1, IL15, IL1RAPL2, IQGAP2, ITGA8, ITGAM, ITGAX, ITPR2, JAZF1, KCNE1, KCNH8, KCTD16, KCTD8, KDM6A, KIF26B,
KRT6B, KRTAP9-9, LALBA, LARS2, LDHC, LDLRAP1, LHX8, LINGO2, LIPG, LRRK2, MASP2, MAST4, MB21D2, MCCC1, MERTK,
mir-548, MMP2, MTG1, MTUS2, MYRIP, NCOA3, NELL1, NFKB1, NMT2, NPHP1, NPR3, NRG3, NSUN3, NSUN6, OCA2, OPHN1,
OSBPL1A, OTOL1, PARD3, PDE11A, PDE4D, PDPN, PDS5B, PDZRN4, PEX5, PHACTR1, phosphate, PKHD1, PLCL1, PLOD2, PLXDC2,
PPP2R2B, PPT1, PRKAG2, PRMT6, PTPN12, PTPRD, PTPRN2, PXDNL, RAB28, RAPGEF2, RAPSN, RHAG, RNF180, RNF19A, ROBO2,
SARDH, SCAPER, SEMA6D, SGCZ, SH3RF3, SIL1, SLC22A20, SLC24A3, SLC25A12, SLC37A2, SLC6A3, SLCO3A1, SMOC2, SPAG17,
SPRED2, SQSTM1, STAG2, STAM, STXBP5L, SUSD1, SYK, SYNE2, TCHH, TDRKH, TEK, TENM2, TFEC, THOP1, THSD7B, TLN2, TMC1,
TNFSF11, TNNI3K, TPST1, TRHDE, TRIM5, TSC22D1, TTC12, TUSC3, UBE2D2, UGGT2, ULK4, UTRN, UVRAG, VAV3, VAX1, VCAM1,
VPS13B, VWC2, WDPCP, WDR27, WWTR1, XK, XKR4, XYLT1, YTHDC2, ZFPM2, ZNF608, ZPLD1
>18
ABCA12, ABCA13, ABCA4, ABCB1, ABCC6, ACSL4, ADAM23, ADAMTS6, ADCY8, AGXT, AKAP9, ALDH5A1, ALOX5, ANK3, ANKRD30A,
ANKS1B, ANO3, ARHGEF4, ATRX, BBS9, BRAF, C12orf42, C4orf22, CADM2, CASP8, CASR, CCDC105, CCDC178, CCDC7, CCSER1, CD22,
CD2AP, CDH10, CDH12, CDH13, CDH2, CENPK, CFH, CLN3, CNBD1, CNTN5, CNTNAP2, COL19A1, CPA4, CPNE4, CPNE8, CPXCR1,
CTNNA3, CTNND2, CUBN, CXCL13, CYP19A1, DAB1, DCC, DLGAP2, DNAH11, DNAH14, DNAI1, DNM3, DOCK4, DYSF, EMB, EPHA7,
ERBB3, ERBB4, ESRRG, EVC, EYS, FAM184A, FAT4, FOXN3, FRAS1, FSTL4, FUT8, GABRA2, GABRB1, GABRB3, GMPR, GOLIM4, GPC5,
GRID1, GRID2, GRIK2, GRM4, GRM5, GRM8, GTDC1, GYS2, HCN1, HIVEP1, HMCN1, HTR3B, IL15, IL1RAPL2, IQGAP2, ITGA8, ITGAM,
ITGAX, ITPR2, JAZF1, KCNE1, KCNH8, KCTD8, KDM6A, KIF26B, KLHL4, KRTAP9-4, LALBA, LHX8, LINGO2, LRRK2, LUZP2,
MACROD2, MASP2, MAST4, MB21D2, MCCC1, MERTK, MFSD8, MMP2, MTUS2, MYRIP, NEK11, NELL1, NKAIN2, NKAIN3, NPHP1,
NPR3, NRG3, NSUN6, OCA2, OPHN1, OSCAR, OTOL1, OXGR1, PAGE4, PDE11A, PDZRN4, PKHD1, PLCL1, PLOD2, PLXDC2, PPARGC1A,
PPP1R14C, PROS1, PTPN22, PTPRD, PTPRN2, PXDNL, RAPGEF2, RIT2, RNF180, ROBO2, SCG3, SGCZ, SH3RF3, SLC24A3, SLCO3A1,
SOD1, SPAG17, SPRED2, STXBP5L, SYK, SYNE2, TCHH, TDRKH, TEK, TENM2, TFEC, THOP1, THSD7B, TLN2, TMC1, TNFSF11,
TNNI3K, TRHDE, TUSC3, UTRN, UVRAG, VAV3, VCAM1, VPS13B, VWC2, WDR27, XKR4, XYLT1, ZBTB49, ZFPM2, ZNF608, ZPLD1
>19
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
ADH1C, AGBL4, AGK, AGXT, AHI1, AKAP9, ALDH3A2, ALDH5A1, ALG8, ALOX5, ANK3, ANKHD30A, ANKRD50, ANKS1B, ANO3,
ARHGAP24, ARHGEF4, ATRX, BBS5, BBS9, BEND7, BMPR2, BNC2, BRAF, C12orf42, C4orf22, CADM2, CAPN3, CASP8, CASR, CCDC105,
CCDC178, CCDC7, CCSER1, CCT8, CD22, CD28, CD2AP, CDH10, CDH12, CDH13, CDH2, CENPK, CES3, CFH, CNBD1, CNTLN, CNTN5,
CNTNAP2, COL19A1, CPA4, CPNE4, CPNE8, CPXCR1, CKHR1, CRYM, CTNNA3, CTNND2, CTSB, CUBN, CWC27, CYBB, CYP19A1,
CYP1A1, CYP3A5, DAB1, DCC, DDAH1, DDIT4, DHRS2, DISC1, DLD, DLGAP2, DMRTA1, DNAH11, DNAH14, DNAJC13, DNM3, DOCK4,
DYM, DYSF, EGR2, EMB, EN1, EPHA7, EPHX1, EPHX2, ERBB3, ERBB4, ESRRG, EVC, EYS, FAF1, FAM122C, FAM172A, FAM184A,
FANCI, FAT4, FEM1C, FGF14, FOXN3, FOXP2, FRAS1, FSTL4, FUT8, GABRA2, GABRB1, GABRB3, GABRG2, GALNT2, GIF, GLRA2,
GMPR, GOLIM4, GPC5, GPR6, GRID1, GRID2, GRIK2, GRM4, GRM5, GRM8, GTDC1, GYS2, HCN1, HGSNAT, HIVEP1, HMCN1, HMGCR,
HMGN5, HPGD, HSD17B4, HYAL1, HYOU1, ICOS, IL15, IL1RAPL1, IL1RAPL2, IMMP2L, IQGAP2, ITGA8, ITGAM, ITGAX, ITPR2, JAZF1,
KCNE1, KCNH8, KCNN2, KCTD16, KCTD8, KDM6A, KIF26B, KLHL3, KLHL4, KRT6B, KRTAP9-4, KRTAP9-8, KRTAP9-9, LALBA, LARS2,
LBR, LDHC, LDLRAP1, LHX8, LINGO2, LIPG, LMBB1, LMO1, LRRK2, LUZP2, MASP2, MAST4, MB21D2, MCCC1, MERTK, mir-548,
MMP2, MS4A4E, MTG1, MTUS2, MYBIP, NCOA3, NEK11, NELL1, NFKB1, NKAIN2, NKAIN3, NMT2, NPHP1, NPR3, NRG3, NSUN3,
NSUN6, NUDT13, OCA2, OPHN1, OSBPL1A, OTOL1, OXGR1, PAGE4, PARD3, PDE11A, PDE4D, PDPN, PDS5B, PDZRN4, PEX5, PHACTR1,
phosphate, PKHD1, PLCL1, PLOD2, PLXDC2, PPP2R2B, PPT1, PRKAG2, PRMT6, PROS1, PRPF8, PTPN12, PTPN22, PTPRD, PTPRN2,
PXDNL, RAB28, RAPGEF2, RAPSN, RHAG, RIT2, RNF180, RNF19A, ROBO2, RPL11, SARDH, SCAPER, SCG3, SEMA6D, SGCZ, SH3RF3,
SIL1, SLC22A20, SLC24A3, SLC25A12, SLC37A2, SLC6A3, SLCO3A1, SMOC2, SOD1, SPAG17, SPRED2, SQSTM1, STAG2, STAM, STXBP5L,
SUOX, SUSD1, SYK, SYNE2, TCHH, TDRKH, TEK, TENM2, TFEC, THOP1, THSD7B, TLN2, TMC1, TNFSF11, TNNI3K, TPST1, TRHDE,
TRIM36, TRIM5, TSC22D1, TTC12 TUSC3, UBE2D2, UGGT2, ULK4, UTRN, UVRAG, VAV3, VAX1, VCAM1, VPS13B, VWC2, WDPCP,
WDR27, WWTR1, XK, XKR4, XYLT1, YTHDC2, ZBTB49, ZFPM2, ZNF608, ZPLD1
>20
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
adenosinetriphosphate, ADGRB3, ADGRL2, ADGRV1, ADH1C, AGBL4, AGK, AGXT, AHI1, AKAP9, ALDH3A2, ALDH5A1, ALG8, ALOX5,
ANK3, ANKRD30A, ANKED50, ANKS1B, ANO3, ARHGAP24, ARHGEF4, ATRX, BBS5, BBSS, BEND7, BMPR2, BNC2, BOD1, BRAF,
C12orf42, C4orf22, CADM2, CAMKMT, CAPN3, CASP8, CASR, CCDC105, CCDC137, CCDC178, CCDC7, CCSER1, CCT8, CD22, CD28,
CD2AP, CDH10, CDH12, CDH13, CDH2, CENPK, CES3, CFH, CHRNA7, CLN3, CNBD1, CNTLN, CNTN5, CNTNAP2, COL19A1, CPA4,
CPNE4, CPNE8, CPXCR1, CRHR1, CRYM, CTNNA3, CTNND2, CTSB, CUBN, CWC27, CXCL13, CYBB, CYP19A1, CYP1A1, CYP3A5, DAB1,
DCC, DDAH1, DDIT4, DHRS2, DISCI, DLD, DLEU1, DLGAP2, DMRTA1, DNAH11, DNAH14, DNAI1, DNAJC13, DNM3, DOCK4, DYM,
DYSF, EGR2, EMB, EN1, EPHA7, EPHX1, EPHX2, ERBB3, ERBB4, ESRRG, EVC, EYS, FAF1, FAM122C, FAM172A, FAM184A, FANCI,
FAT4, FEM1C, FGF14, FOXN3, FOXP2, FRAS1, FSTL4, FUT8, GABRA2, GABRB1, GABRB3, GABRG2, GALNT2, GIF, GLRA2, GMPR,
GOLIM4, GPC5, GPR6, GRID1, GRID2, GRIK2, GRM4, GRM5, GRM8, GSTM2, GTDC1, GYS2, HCN1, HGSNAT, HIVEP1, HMCN1, HMGCR,
HMGN5, HPGD, HSD17B4, HTR3B, HYOU1, ICOS, IL15, IL1RAPL1, IL1RAPL2, IMMP2L, IQGAP2, ITGA8, ITGAM, ITGAX, ITPR2, JAZF1,
KCNE1, KCNH8, KCNN2, KCTD16, KCTD8, KDM6A, KIF26B, KLHL3, KLHL4, KRT6B, KRTAP9-2, KRTAP9-4, KRTAP9-8, KRTAP9-9,
LALBA, LARS2, LBR, LDIIC, LDLRAP1, LIIX8, LINGO2, LIPG, LMBR1, LMO1, LRRK2, LUZP2, MACROD2, MASP2, MAST4, MB21D2,
MCCC1, MERTK, MFSD8, mir-548, MMP2, MS4A4E, MTG1, MTUS2, MYRIP, NCOA3, NEK11, NELL1, NFKB1, NKAIN2, NKAIN3, NMT2,
NPHP1, NPR3, NRG3, NSUN3, NSUN6, NUDT13, OCA2, OPHN1, OSBPL1A, OSCAR, OTOL1, OXGR1, PAGE4, PARD3, PDE11A, PDE4D,
PDPN, PDS5B, PDZRN4, PEX5, PHACTR1, phosphate, PKHD1, PLCL1, PLOD2, PLXDC2, PPARGC1A, PPP1R14C, PPP2R2B, PPT1, PRKAG2,
PRMT6, PROS1, PRPF8, PTPN12, PTPN22, PTPRD, PTPRN2, PVT1, PXDNL, RAB28, RAPGEF2, RAPSN, RIIAG, RIT2, RNF180, RNF19A,
ROBO2, RPL11, SARDH, SCAPER, SCG3, SEMA6D, SGCZ, SH3RF3, SIL1, SLC22A20, SLC24A3, SLC25A12, SLC37A2, SLC6A3, SLCO3A1,
SMOC2, SOD1, SPAG17, SPRED2, SQSTM1, STAG2, STAM, STXBP5L, SUOX, SUSD1, SYK, SYNE2, TCHH, TDRKH, TEK, TENM2, TFEC,
THOP1, THSD7B, TLN2, TMC1, TNFSF11, TNNI3K, TPST1, TRHDE, TRIM36, TRIM5, TSC22D1, TTC12, TUSC3, UBE2D2, UGGT2, ULK4,
UTRN, UVRAG, VAV3, VAX1, VCAM1, VPS13B, VWC2, WDPCP, WDR27, WWTR1, XK, XKR4, XYLT1, YTIIDC2, ZBTB49, ZFPM2, ZNF608,
ZPLD1
>21
ABCA10, ABCA12, ABCA13, ABCA4, ABCB1, ABCC6, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BNC2, BRAF, C12orf42, C4orf22, CASP8, CASR, CCDC7, CDII10, CDII12,
CDH13, CNTN5, CNTNAP2, COL19A1, CTNNA3, CTNND2, CUBN, CYBB, CYP19A1, CYP1A1, CYP3A5, DCC, DISC1, DLGAP2, DNAH11,
DNAH14, DNAJC13, DOCK4, DYM, DYSF, EPHA7, ERBB3, ERBB4, EYS, FAT4, FOXP2, FUT8, GABRA2, GABRB1, GABRG2, GIF, GMPR,
GRM4, GRM5, GRM8, GTDC1, HCN1, HMCN1, HMGN5, IL1RAPL1, IMMP2L, ITGAM, ITPR2, KCNH8, KDM6A, KRT6B, LHX8, LINGO2,
LRRK2, MAST4, MMP2, MTG1, MTUS2, NEK11, NRG3, OPIIN1, OXGR1, PAGE4, PARD3, PDE4D, PDS5B, PDZRN4, PKIID1, PLCL1,
PPP2R2B, PRKAG2, PROS1, PRPF8, PTPN22, PTPRD, PTPRN2, PXDNL, RAPGEF2, ROBO2, SCAPER, SEMA6D, SLC24A3, SLC25A12,
SLCO3A1, SPRED2, STAG2, STXBP5L, SUSD1, SYNE2, TCHH, TEK, THOP1, THSD7B, TLN2, TNNI3K, TRHDE, TSC22D1, TUSC3, UGGT2,
ULK4, UTRN, UVRAG, VPS13B, WWTR1, XKR4, XYLT1, YTHDC2, ZFPM2
>22
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
AGBL4, AGXT, AHI1, AKAP9, ALDH3A2, ALG8, ALOX5, ANK3, ANKRD30A, ANKRD50, ANKS1B, ARHGAP24, ARHGEF4, ATRX, BBS9,
BMPR2, BNC2, BRAF, C12orf42, C4orf22, CAPN3, CASP8, CASR, CCDC105, CCDC178, CCDC7, CCSER1, CCT8, CD22, CD28, CD2AP,
CDH10, CDH12, CDH13, CDH2, CENPK, CES3, CFH, CNBD1, CNTLN, CNTN5, CNTNAP2, COL19A1, CPA4, CPNE4, CPNE8, CTNNA3,
CTNND2, CTSB, CUBN, CWC27, CYBB, CYP19A1, CYP3A5, DAB1, DCC, DDIT4, DISC1, DLD, DLGAP2, DMRTA1, DNAH11, DNAH14,
DNAJC13, DNM3, DOCK4, DYM, DYSF, EGR2, EN1, EPHA7, EPHX1, EPHX2, ERBB3, ERBB4, EVC, EYS, FAM122C, FAM172A, FANCI,
FAT4, FEM1C, FGF14, FOXN3, FOXP2, FRAS1, FSTL4, FUT8, GABRA2, GABRB3, GALNT2, GPC5, GPR6, GRID1, GRID2, GRIK2, GRM4,
GRM5, GRM8, GTDC1, GYS2, HCN1, HGSNAT, HIVEP1, HMCN1, HMGCR, HMGN5, HPGD, HSD17B4, HYOU1, IL15, IL1RAPL2, IQGAP2,
ITGA8, ITGAM, ITGAX, ITPR2, JAZF1, KCNE1, KCNH8, KCTD16, KCTD8, KDM6A, KIF26B, KRT6B, KRTAP9-9, LALBA, LARS2, LDHC,
LDLRAP1, LHX8, LINGO2, LIPG, LRRK2, MASP2, MAST4, MB21D2, MCCC1, MERTK, mir-548, MMP2, MTG1, MTUS2, MYRIP, NCOA3,
NELL1, NMT2, NPHP1, NPR3, NRG3, NSUN3, NSUN6, OCA2, OSBPL1A, OTOL1, PARD3, PDE11A, PDE4D, PDPN, PDS5B, PDZRN4,
PEX5, PHACTR1, phosphate, PKHD1, PLCL1, PLOD2, PLXDC2, PPP2R2B, PPT1, PRKAG2, PRMT6, PTPN12, PTPRD, PTPRN2, PXDNL,
RAB28, RAPGEF2, RAPSN, RHAG, RNF180, RNF19A, ROBO2, SARDH, SCAPER, SH3RF3, SIL1, SLC22A20, SLC24A3, SLC25A12, SLC37A2,
SLCO3A1, SMOC2, SPAG17, SPRED2, SQSTM1, STAG2, STAM, STXBP5L, SUSD1, SYK, SYNE2, TCHH, TEK, TENM2, TFEC, THOP1,
THSD7B, TLN2, TMC1, TNFSF11, TNN13K, TPST1, TRHDE, TRIM5, TSC22D1, TTC12, UBE2D2, UGGT2, ULK4, UTRN, UVRAG, VAV3,
VAX1, VCAM1, VPS13B, VWC2, WDPCP, WDR27, WWTR1, XK, XKR4, XYLT1, YTHDC2, ZFPM2, ZNF608, ZPLD1
>23
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
AGBL4, AGXT, AHI1, AKAP9, ALDH3A2, ALG8, ALOX5, ANK3, ANKRD30A, ANKRD50, ANKS1B, ARHGAP24, ARHGEF4, ATRX, BBS9,
BMPR2, BNC2, BRAF, C12orf42, C4orf22, CAPN3, CASP8, CASR, CCDC105, CCDC178, CCDC7, CCSER1, CCT8, CD22, CD28, CD2AP,
CDH10, CDH12, CDH13, CDH2, CENPK, CES3, CFH, CNBD1, CNTLN, CNTN5, CNTNAP2, COL19A1, CPA4, CPNE4, CPNE8, CTNNA3,
CTNND2, CTSB, CUBN, CWC27, CYBB, CYP19A1, CYP3A5, DAB1, DCC, DDIT4, DISC1, DLD, DLGAP2, DMRTA1, DNAH11, DNAH14,
DNAJC13, DNM3, DOCK4, DYM, DYSF, EGR2, EN1, EPHA7, EPHX1, EPHX2, ERBB3, ERBB4, EVC, EYS, FAM122C, FAM172A, FANCI,
FAT4, FEM1C, FGF14, FOXN3, FOXP2, FRAS1, FSTL4, FUT8, GABRA2, GABRB3, GALNT2, GPC5, GPR6, GRID1, GRID2, GRIK2, GRM4,
GRM5, GRM8, GTDC1, GYS2, HCN1, HGSNAT, HIVEP1, HMCN1, HMGCR, HMGN5, HPGD, HSD17B4, HYOU1, IL15, IL1RAPL2, IQGAP2,
ITGA8, ITGAM, ITGAX, ITPR2, JAZF1, KCNE1, KCNH8, KCTD16, KCTD8, KDM6A, KIF26B, KRT6B, KRTAP9-9, LALBA, LARS2, LDHC,
LDLRAP1, LHX8, LINGO2, LIPG, LRRK2, MASP2, MAST4, MB21D2, MCCC1, MERTK, mir-548, MMP2, MTG1,
MTUS2, MYRIP, NCOA3, NELL1, NMT2, NPHP1, NPR3, NRG3, NSUN3, NSUN6, OCA2, OSBPL1A, OTOL1, PARD3, PDE11A, PDE4D,
PDPN, PDS5B, PDZRN4, PEX5, PHACTR1, phosphate, PKHD1, PLCL1, PLOD2, PLXDC2, PPP2R2B, PPT1, PRKAG2, PRMT6, PTPN12,
PTPRD, PTPRN2, PXDNL, RAB28, RAPGEF2, RAPSN, RHAG, RNF180, RNF19A, ROBO2, SARDH, SCAPER, SH3RF3, SIL1, SLC22A20,
SLC24A3, SLC25A12, SLC37A2, SLCO3A1, SMOC2, SPAG17, SPRED2, SQSTM1, STAG2, STAM, STXBP5L, SUSD1, SYK, SYNE2, TCHH,
TEK, TENM2, TFEC, THOP1, THSD7B, TLN2, TMC1, TNFSF11, TNNI3K, TPST1, TRHDE, TRIM5, TSC22D1, TTC12, UBE2D2, UGGT2,
UTRN, UVRAG, VAV3, VAX1, VCAM1, VPS13B, VWC2, WDPCP, WDE27, WWTR1, XK, XKR4, XYLT1, YTHDC2, ZFPM2, ZNF608, ZPLD1
>24
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
ADH1C, AGBL4, AGK, AGXT, AHI1, AKAP9, ALDH3A2, ALDH5A1, ALG8, ALOX5, ANK3, ANKRD30A, ANKRD50, ANKS1B, ANO3,
ARHGAP24, ARHGEF4, ATRX, BBS5, BBS9, BEND7, BMPR2, BNC2, BRAF, C12orf42, C4orf22, CADM2, CAPN3, CASP8, CASR, CCDC105,
CCDC178, CCDC7, CCSER1, CCT8, CD22, CD28, CD2AP, CDH10, CDH12, CDH13, CDH2, CENPK, CES3, CFH, CNBD1, CNTLN, CNTN5,
CNTNAP2, COL19A1, CPA4, CPNE4, CPNE8, CPXCR1, CRHR1, CRYM, CTNNA3, CTNND2, CTSB, CUBN, CWC27, CYBB, CYP19A1,
CYP1A1, CYP3A5, DAB1, DCC, DDAH1, DDIT4, DHRS2, DISC1, DLD, DLGAP2, DMRTA1, DNAH11, DNAH14, DNAJC13, DNM3, DOCK4,
DYM, DYSF, EGR2, EMB, EN1, EPHA7, EPHX1, EPHX2, ERBB3, ERBB4, ESRRG, EVC, EYS, FAF1, FAM122C, FAM172A, FAM184A,
FANCI, FAT4, FEM1C, FGF14, FOXN3, FOXP2, FRAS1, FSTL4, FUT8, GABRA2, GABRB1, GABRB3, GABRG2, GALNT2, GIF, GLRA2,
GMPR, GOLIM4, GPC5, GPR6, GRID1, GRID2, GRIK2, GRM4, GRM5, GRM8, GTDC1, GYS2, HCN1, HGSNAT, HIVEP1, HMCN1, HMGCR,
HMGN5, HSD17B4, HYOU1, ICOS, IL15, IL1RAPL1, IL1RAPL2, IMMP2L, IQGAP2, ITGA8, ITGAM, ITGAX, ITPR2, JAZF1, KCNE1, KCNH8,
KCNN2, KCTD16, KCTD8, KDM6A, KIF26B, KLHL3, KLHL4, KRT6B, KRTAP9-4, KRTAP9-9, LALBA, LARS2, LBR, LDHC, LDLRAP1,
LHX8, LINGO2, LIPG, LMBR1, LMO1, LRRK2, LUZP2, MASP2, MAST4, MB21D2, MCCC1, MERTK, mir-548, MMP2, MS4A4E, MTG1,
MTUS2, MYRIP, NCOA3, NEK11, NELL1, NFKB1, NKAIN2, NKAIN3, NMT2, NPHP1, NPR3, NRG3, NSUN3, NSUN6, NUDT13, OCA2,
OPHN1, OSBPL1A, OTOL1, OXGR1, PAGE4, PARD3, PDE11A, PDE4D, PDPN, PDS5B, PDZRN4, PEX5, PHACTR1, phosphate, PKHD1,
PLCL1, PLOD2, PLXDC2, PPP2R2B, PPT1, PRKAG2, PRMT6, PROS1, PRPF8, PTPN12, PTPN22, PTPRD, PTPRN2, PXDNL, RAB28,
RAPGEF2, RAPSN, RHAG, RIT2, RNF180, RNF19A, ROBO2, RPL11, SARDH, SCAPER, SCG3, SEMA6D, SGCZ, SH3RF3, SIL1, SLC22A20,
SLC24A3, SLC25A12, SLC37A2, SLC6A3, SLCO3A1, SMOC2, SOD1, SPAG17, SPRED2, SQSTM1, STAG2, STAM, STXBP5L, SUOX, SUSD1,
SYK, SYNE2, TCHH, TDRKH, TEK, TENM2, TFEC, THOP1, THSD7B, TLN2, TMC1, TNFSF11, TNNI3K, TPST1, TRHDE, TRIM36, TRIM5,
TSC22D1, TTC12, TUSC3, UBE2D2, UGGT2, ULK4, UTRN, UVRAG, VAV3, VAX1, VCAM1, VPS13B, VWC2, WDPCP, WDR27, WWTR1, XK,
XKR4, XYLT1, YTHDC2, ZBTB49, ZFPM2, ZNF608, ZPLD1
>25
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
ADH1C, AGBL4, AGK, AGXT, AHI1, AKAP9, ALDH3A2, ALDH5A1, ALG8, ALOX5, ANK3, ANKRD30A, ANKRD50, ANKS1B, ANO3,
ARHGAP24, ARHGEF4, ATRX, BBS5, BBS9, BEND7, BMPR2, BNC2, BOD1, BRAF, C12orf42, C4orf22, CADM2, CAMKMT, CASP8, CASR,
CCDC105, CCDC178, CCDC7, CCSER1, CCT8, CD22, CD28, CD2AP, CDH10, CDH12, CDH13, CDH2, CENPK, CES3, CFH, CHRNA7, CLN3,
CNBD1, CNTLN, CNTN5, CNTNAP2, COL19A1, CPA4, CPNE4, CPNE8, CPXCR1, CRHR1, CRYM, CTNNA3, CTNND2, CTSB, CUBN,
CWC27, CXCL13, CYBB, CYP19A1, CYP1A1, CYP3A5, DAB1, DCC, DDAH1, DDIT4, DHRS2, DISC1, DLD, DLGAP2, DMRTA1, DNAH11,
DNAH14, DNAJC13, DNM3, DOCK4, DYM, DYSF, EGR2, EMB, EN1, EPHA7, EPHX1, EPHX2, ERBB3, ERBB4, ESRRG, EVC, EYS, FAF1,
FAM122C, FAM172A, FAM184A, FANCI, FAT4, FEM1C, FGF14, FOXN3, FOXP2, FRAS1, FSTL4, FUT8, GABRA2, GABRB1, GABRB3,
GABRG2, GALNT2, GIF, GLRA2, GMPR, GOLIM4, GPC5, GPR6, GRID1, GRID2, GRIK2, GRM4, GRM5, GRM8, GTDC1, GYS2, HCN1,
HGSNAT, HIVEP1, HMCN1, HMGCR, HMGN5, HPGD, HSD17B4, HYOU1, ICOS, IL1RAPL1, IL1RAPL2, IMMP2L, IQGAP2, ITGA8, ITGAM,
ITGAX, ITPR2, JAZF1, KCNE1, KCNH8, KCNN2, KCTD16, KCTD8, KDM6A, KIF26B, KLHL3, KLHL4, KRT6B, KRTAP9-2, KRTAP9-4,
KRTAP9-8, KRTAP9-9, LALBA, LARS2, LBR, LDHC, LDLRAP1, LHX8, LINGO2, LIPG, LMBR1, LMO1, LRRK2, LUZP2, MASP2, MAST4,
MB21D2, MCCC1, MERTK, MMP2, MS4A4E, MTG1, MTUS2, MYRIP, NCOA3, NEK11, NELL1, NFKB1, NKAIN2, NKAIN3, NMT2, NPHP1,
NPR3, NRG3, NSUN3, NSUN6, NUDT13, OCA2, OPHN1, OSBPL1A, OTOL1, OXGR1, PAGE4, PARD3, PDE11A, PDE4D, PDPN, PDS5B,
PDZRN4, PEX5, PHACTR1, phosphate, PKHD1, PLCL1, PLOD2, PLXDC2, PPARGC1A, PPP2R2B, PPT1, PRKAG2, PRMT6, PROS1, PRPF8,
PTPN12, PTPN22, PTPRD, PTPRN2, PXDNL, RAB28, RAPGEF2, RAPSN, RHAG, RIT2, RNF180, RNF19A, ROBO2, RPL11, SARDH,
SCAPER, SCG3, SEMA6D, SGCZ, SH3RF3, SIL1, SLC22A20, SLC24A3, SLC25A12, SLC37A2, SLC6A3, SLCO3A1, SMOC2, SOD1, SPAG17,
SPRED2, SQSTM1, STAG2, STAM, STXBP5L, SUOX, SUSD1, SYK, SYNE2, TCHH, TDRKH, TEK, TENM2, TFEC, THOP1, THSD7B, TLN2,
TMC1, TNFSF11, TNNI3K, TPST1, TRHDE, TRIM36, TRIM5, TSC22D1, TTC12, TUSC3, UBE2D2, UGGT2, ULK4, UTRN, UVRAG, VAV3,
VAX1, VCAM1, VPS13B, VWC2, WDPCP, WDR27, WWTR1, XK, XKR4, XYLT1, YTIIDC2, ZBTB49, ZFPM2, ZNF608, ZPLD1
>26
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
AGBL4, AGXT, AHI1, AKAP9, ALDH3A2, ALG8, ALOX5, ANK3, ANKRD30A, ANKRD50, ANKS1B, ARHGAP24, ARHGEF4, ATRX, BBS9,
BMPR2, BNC2, BRAF, C12orf42, C4orf22, CAPN3, CASP8, CASR, CCDC105, CCDC178, CCDC7, CCSER1, CCT8, CD22, CD28, CD2AP,
CDH10, CDH12, CDH13, CDH2, CENPK, CES3, CFH, CNBD1, CNTLN, CNTN5, CNTNAP2, COL19A1, CPA4, CPNE4, CPNE8, CTNNA3,
CTNND2, CTSB, CUBN, CWC27, CYBB, CYP19A1, CYP3A5, DAB1, DCC, DDIT4, DISC1, DLD, DLGAP2, DMRTA1, DNAH11, DNAH14,
DNAJC13, DNM3, DOCK4, DYM, DYSF, EGR2, EN1, EPHA7, EPHX1, EPHX2, ERBB3, ERBB4, EVC, EYS, FAM122C, FAM172A, FANCI,
FAT4, FEM1C, FGF14, FOXN3, FOXP2, FRAS1, FSTL4, FUT8, GABRA2, GABRB3, GALNT2, GPC5, GPR6, GRID1, GRID2, GRIK2, GRM4,
GRM5, GRM8, GTDC1, GYS2, HCN1, HGSNAT, HIVEP1, HMCN1, HMGCR, HMGN5, HSD17B4, HYOU1, IL15, IL1RAPL2, IQGAP2, ITGA8,
ITGAM, ITGAX, ITPR2, JAZF1, KCNE1, KCNH8, KCTD16, KCTD8, KDM6A, KIF26B, KRT6B, KRTAP9-9, LALBA, LARS2, LDHC, LDLRAP1,
LHX8, LINGO2, LIPG, LRRK2, MASP2, MAST4, MB21D2, MCCC1, MERTK, mir-548, MMP2, MTG1, MTUS2, MYRIP, NCOA3, NELL1,
NMT2, NPIIP1, NPR3, NRG3, NSUN3, NSUN6, OCA2, OSBPL1A, OTOL1, PARD3, PDE11A, PDE4D, PDS5B, PDZRN4, PEX5, PIIACTR1,
phosphate, PKHD1, PLCL1, PLOD2, PLXDC2, PPP2R2B, PPT1, PRKAG2, PRMT6, PTPN12, PTPRD, PTPRN2, PXDNL, RAB28, RAPGEF2,
RAPSN, RHAG, RNF180, RNF19A, ROBO2, SARDH, SCAPER, SH3RF3, SIL1, SLC22A20, SLC24A3, SLC25A12, SLC37A2, SLCO3A1, SMOC2,
SPAG17, SPRED2, SQSTM1, STAG2, STAM, STXBP5L, SUSD1, SYK, SYNE2, TCHH, TEK, TENM2, TFEC, THOP1, THSD7B, TLN2,
TMC1, TNFSF11, TNNI3K, TPST1, TRIIDE, TRIM5, TSC22D1, TTC12, UBE2D2, UGGT2, ULK4, UTRN, UVRAG, VAV3, VAX1, VCAM1,
VPS13B, VWC2, WDPCP, WDR27, WWTR1, XK, XKR4, XYLT1, YTHDC2, ZFPM2, ZNF608, ZPLD1
>27
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
AGBL4, AGXT, AHI1, AKAP9, ALDH3A2, ALG8, ALOX5, ANK3, ANKRD30A, ANKRD50, ANKS1B, ARHGAP24, ARHGEF4, ATRX, BBS9,
BMPR2, BNC2, BRAF, C12orf42, C4orf22, CAPN3, CASP8, CASR, CCDC105, CCDC178, CCDC7, CCSER1, CCT8, CD22, CD28, CD2AP,
CDH10, CDH12, CDH13, CDH2, CENPK, CES3, CFH, CNBD1, CNTLN, CNTN5, CNTNAP2, COL19A1, CPA4, CPNE4, CPNE8, CTNNA3,
CTNND2, CTSB, CUBN, CWC27, CYBB, CYP19A1, CYP3A5, DAB1, DCC, DDIT4, DISC1, DLD, DLGAP2, DMRTA1, DNAH11, DNAH14,
DNAJC13, DNM3, DOCK4, DYM, DYSF, EGR2, EN1, EPHA7, EPHX1, EPHX2, ERBB3, ERBB4, EVC, EYS, FAM122C, FAM172A, FANCI,
FAT4, FEM1C, FGF14, FOXN3, FOXP2, FRAS1, FSTL4, FUT8, GABRA2, GABRB3, GALNT2, GPC5, GPR6, GRID1, GRID2, GRIK2, GRM4,
GRM5, GRM8, GTDC1, GYS2, HCN1, HGSNAT, HIVEP1, HMCN1, HMGCR, HMGN5, HSD17B4, HYOU1, IL15, IL1RAPL2, IQGAP2, ITGA8,
ITGAM, ITGAX, ITPR2, JAZF1, KCNE1, KCNH8, KCTD16, KCTD8, KDM6A, KIF26B, KRT6B, KRTAP9-9, LALBA, LARS2, LDHC, LDLRAP1,
LHX8, LINGO2, LIPG, LRRK2, MASP2, MAST4, MB21D2, MCCC1, MERTK, mir-548, MMP2, MTG1, MTUS2, MYRIP, NCOA3, NELL1,
NMT2, NPHP1, NPR3, NRG3, NSUN3, NSUN6, OCA2, OSBPL1A, OTOL1, PARD3, PDE11A, PDE4D, PDS5B, PDZRN4, PEX5, PHACTR1,
phosphate, PKHD1, PLCL1, PLOD2, PLXDC2, PPP2R2B, PPT1, PRKAG2, PRMT6, PTPN12, PTPRD, PTPRN2, PXDNL, RAB28, RAPGEF2,
RAPSN, RHAG, RNF180, RNF19A, ROBO2, SARDH, SCAPER, SH3RF3, SIL1, SLC22A20, SLC24A3, SLC25A12, SLC37A2, SLCO3A1, SMOC2,
SPAG17, SPRED2, SQSTM1, STAG2, STAM, STXBP5L, SUSD1, SYK, SYNE2, TCHH, TEK, TENM2, TFEC, THOP1, THSD7B, TLN2,
TMC1, TNFSF11, TNNI3K, TPST1, TRHDE, TRIM5, TSC22D1, TTC12, UBE2D2, UGGT2, UTRN, UVRAG, VAV3, VAX1, VCAM1, VPS13B,
VWC2, WDPCP, WDR27, WWTR1, XK, XKR4, XYLT1, YTHDC2, ZFPM2, ZNF608, ZPLD1
>28
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
ADH1C, AGBL4, AGK, AGXT, AHI1, AKAP9, ALDH3A2, ALDH5A1, ALG8, ALOX5, ANK3, ANKRD30A, ANKRD50, ANKS1B, ANO3,
ARHGAP24, ARHGEF4, ATRX, BBS5, BBS9, BEND7, BMPR2, BNC2, BOD1, BRAF, C12orf42, C4orf22, CADM2, CAMKMT, CASP8, CASR,
CCDC105, CCDC178, CCDC7, CCSER1, CCT8, CD22, CD28, CD2AP, CDH10, CDH12, CDH13, CDH2, CENPK, CES3, CFH, CHRNA7, CLN3,
CNBD1, CNTLN, CNTN5, CNTNAP2, COL19A1, CPA4, CPNE4, CPNE8, CPXCR1, CRHR1, CRYM, CTNNA3, CTNND2, CTSB, CUBN,
CWC27, CXCL13, CYBB, CYP19A1, CYP1A1, CYP3A5, DAB1, DCC, DDAH1, DDIT4, DHRS2, DISC1, DLD, DLGAP2, DMRTA1, DNAH11,
DNAH14, DNAJC13, DNM3, DOCK4, DYM, DYSF, EGR2, EMB, EN1, EPHA7, EPHX1, EPHX2, ERBB3, ERBB4, ESRRG, EVC, EYS, FAF1,
FAM122C, FAM172A, FAM184A, FANCI, FAT4, FEM1C, FGF14, FOXN3, FOXP2, FRAS1, FSTL4, FUT8, GABRA2, GABRB1, GABRB3,
GABRG2, GALNT2, GIF, GMPR, GOLIM4, GPC5, GPR6, GRID1, GRID2, GRIK2, GRM4, GRM5, GRM8, GTDC1, GYS2, HCN1, HGSNAT,
HIVEP1, HMCN1, HMGCR, HMGN5, HPGD, HSD17B4, HYOU1, ICOS, IL1RAPL1, IL1RAPL2, IMMP2L, IQGAP2, ITGA8, ITGAM, ITGAX,
ITPR2, JAZF1, KCNE1, KCNH8, KCNN2, KCTD16, KCTD8, KDM6A, KIF26B, KLHL3, KLHL4, KRT6B, KRTAP9-2, KRTAP9-4, KRTAP9-8,
KRTAP9-9, LALBA, LARS2, LBR, LDHC, LDLRAP1, LHX8, LINGO2, LIPG, LMBR1, LMO1, LRRK2, LUZP2, MASP2, MAST4, MB21D2,
MCCC1, MERTK, MMP2, MS4A4E, MTG1, MTUS2, MYRIP, NCOA3, NEK11, NELL1, NFKB1, NKAIN2, NKAIN3, NMT2, NPHP1, NPR3,
NRG3, NSUN3, NSUN6, NUDT13, OCA2, OPHN1, OSBPL1A, OTOL1, OXGR1, PAGE4, PARD3, PDE11A, PDE4D, PDPN, PDS5B, PDZRN4,
PEX5, PHACTR1, PKHD1, PLCL1, PLOD2, PLXDC2, PPARGC1A, PPP2R2B, PPT1, PRKAG2, PRMT6, PROS1, PRPF8, PTPN12, PTPN22,
PTPRD, PTPRN2, PXDNL, RAB28, RAPGEF2, RAPSN, RHAG, RIT2, RNF180, RNF19A, ROBO2, RPL11, SARDH, SCAPER, SCG3, SEMA6D,
SGCZ, SH3RF3, SIL1, SLC22A20, SLC24A3, SLC25A12, SLC37A2, SLC6A3, SLCO3A1, SMOC2, SOD1, SPAG17, SPRED2, SQSTM1, STAG2,
STAM, STXBP5L, SUOX, SUSD1, SYK, SYNE2, TCHH, TDRKH, TEK, TENM2, TFEC, THOP1, THSD7B, TLN2, TMC1, TNFSF11, TNNI3K,
TPST1, TRHDE, TRIM36, TRIM5, TSC22D1, TTC12, TUSC3, UBE2D2, UGGT2, ULK4, UTRN, UVBAG, VAV3, VAX1, VCAM1, VPS13B,
VWC2, WDPCP, WDR27, WWTR1, XK, XKR4, XYLT1, YTHDC2, ZBTB49, ZFPM2, ZNF608, ZPLD1
>29
ABCA10, ABCA12, ABCA13, ABCA4, ABCB1, ABCC6, ACADS, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BNC2, BRAF, C12orf42, C4orf22, CASP8, CASR, CCDC7, CDH10, CDH12,
CDH2, CFH, CNTN5, CNTNAP2, COL19A1, CTNNA3, CTNND2, CUBN, CYBB, CYP1A1, CYP3A5, DCC, DISC1, DLGAP2, DNAH11,
DNAH14, DNAJC13, DNM3, DOCK4, DYM, DYSF, EGR2, EPHA7, EPHX1, ERBB3, ERBB4, EVC, EYS, FAT4, FOXP2, FUT8, GABRA2,
GABRB1, GABRG2, GIF, GMPR, GRID1, GRM4, GRM5, GRM8, GTDC1, HCN1, HMCN1, HMGN5, IL1RAPL1, IMMP2L, ITGAM, ITGAX,
ITPR2, KCNH8, KDM6A, KRT6B, LHX8, LINGO2, LRRK2, MAST4, MTG1, MTUS2, NCOA3, NEK11, NRG3, OPHN1, OSBPL1A, OXGR1,
PAGE4, PARD3, PDE4D, PDS5B, PDZRN4, PKHD1, PLCL1, PPP2R2B, PRKAG2, PROS1, PRPF8, PTPN22, PTPRD, PTPRN2, PXDNL,
RAPGEF2, ROBO2, SCAPER, SEMA6D, SLC24A3, SLC25A12, SLCO3A1, SPRED2, STAG2, STXBP5L, SUSD1, SYNE2, TCHH, TEK, TENM2,
THOP1, THSD7B, TLN2, TNNI3K, TRHDE, TRIM36, TSC22D1, TUSC3, UGGT2, ULK4, UTRN, UVRAG, VPS13B, WDPCP, WWTR1, XKR4,
YTHDC2, ZFPM2
>30
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
ADH1C, AGBL4, AGK, AGXT, AHI1, AKAP9, ALDH3A2, ALDH5A1, ALG8, ALOX5, ANK3, ANKRD30A, ANKRD50, ANKS1B, ANO3,
ARHGAP24, ARHGEF4, ATRX, BBS5, BBS9, BEND7, BMPR2, BNC2, BOD1, BRAF, C12orf42, C4orf22, CADM2, CAMKMT, CASP8, CASR,
CCDC105, CCDC178, CCDC7, CCSER1, CCT8, CD22, CD28, CD2AP, CDH10, CDH12, CDH13, CDH2, CENPK, CES3, CFH, CHRNA7, CLN3,
CNBD1, CNTLN, CNTN5, CNTNAP2, COL19A1, CPA4, CPNE4, CPNE8, CPXCR1, CRHR1, CRYM, CTNNA3, CTNND2, CTSB, CUBN,
CWC27, CXCL13, CYBB, CYP19A1, CYP1A1, CYP3A5, DAB1, DCC, DDAH1, DDIT4, DHRS2, DISC1, DLD, DLGAP2, DMRTA1, DNAH11,
DNAH14, DNAJC13, DNM3, DOCK4, DYM, DYSF, EGR2, EMB, EN1, EPHA7, EPHX1, EPHX2, ERBB3, ERBB4, ESRRG, EVC, EYS, FAF1,
FAM122C, FAM172A, FAM184A, FANCI, FAT4, FEM1C, FGF14, FOXN3, FOXP2, FRAS1, FSTL4, FUT8, GABRA2, GABRB1, GABRB3,
GABRG2, GALNT2, GIF, GLRA2, GMPR, GOLIM4, GPC5, GPR6, GRID1, GRID2, GRIK2, GRM4, GRM5, GRM8, GTDC1, GYS2, HCN1,
HGSNAT, HIVEP1, HMCN1, HMGCR, HMGN5, HPGD, HSD17B4, HYOU1, ICOS, IL15, IL1RAPL1, IL1RAPL2, IMMP2L, IQGAP2, ITGA8,
ITGAM, ITGAX, ITPR2, JAZF1, KCNE1, KCNH8, KCNN2, KCTD16, KCTD8, KDM6A, KIF26B, KLHL3, KLHL4, KRT6B, KRTAP9-2,
KRTAP9-4, KRTAP9-8, KRTAP9-9, LALBA, LARS2, LBR, LDHC, LDLRAP1, LHX8, LINGO2, LIPG, LMBR1, LMO1, LRRK2, LUZP2, MASP2,
MAST4, MB21D2, MCCC1, MERTK, MMP2, MS4A4E, MTG1, MTUS2, MYRIP, NCOA3, NEK11, NELL1, NFKB1, NKAIN2, NKAIN3, NMT2,
NPHP1, NPR3, NRG3, NSUN3, NSUN6, NUDT13, OCAS, OPHN1, OSBPL1A, OTOL1, OXGR1, PAGE4, PARD3, PDE11A, PDE4D, PDPN,
PDS5B, PDZRN4, PEX5, PHACTR1, phosphate, PKHD1, PLCL1, PLOD2, PLXDC2, PPARGC1A, PPP2R2B, PPT1, PRKAG2, PRMT6, PROS1,
PRPF8, PTPN12, PTPN22, PTPRD, PTPRN2, PXDNL, RAB28, RAPGEF2, RAPSN, RHAG, RIT2, RNF180, RNF19A, ROBO2, RPL11, SARDH,
SCAPER, SCG3, SEMA6D, SGCZ, SH3RF3, SIL1, SLC22A20, SLC24A3, SLC25A12, SLC37A2, SLC6A3, SLCO3A1, SMOC2, SOD1, SPAG17,
SPRED2, SQSTM1, STAG2, STAM, STXBP5L, SUOX, SUSD1, SYK, SYNE2, TCHH, TDRKH, TEK, TENM2, TFEC, THOP1, THSD7B, TLN2,
TMC1, TNFSF11, TNNI3K, TPST1, TRHDE, TRIM36, TRIM5, TSC22D1, TTC12, TUSC3, UBE2D2, UGGT2, ULK4, UTRN, UVRAG, VAV3,
VAX1, VCAM1, VPS13B, VWC2, WDPCP, WDR27, WWTR1, XK, XKR4, XYLT1, YTHDC2, ZBTB49, ZFPM2, ZNF608, ZPLD1
>31
ABCA10, ABCA12, ABCA13, ABCA4, ABCB1, ABCC6, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BNC2, BRAF, C12orf42, C4orf22, CASP8, CASR, CCDC7, CDH10, CDH12,
CDH13, CDH2, CNTN5, CNTNAP2, COL19A1, CTNNA3, CTNND2, CTSB, CUBN, CYBB, CYP19A1, CYP1A1, CYP3A5, DCC, DISC1,
DLGAP2, DNAH11, DNAH14, DNAJC13, DOCK4, DYM, DYSF, EPHA7, ERBB3, ERBB4, EYS, FAT4, FOXP2, FLIT8, GABRA2, GABRB1,
GABRG2, GIF, GMPR, GRM4, GRM5, GRM8, GTDC1, HCN1, HMCN1, HMGN5, IL1RAPL1, IMMP2L, ITGAM, ITGAX, ITPR2, KCNH8,
KDM6A, KRT6B, LHX8, LINGO2, LRRK2, MAST4, MB21D2, MMP2, MTG1, MTUS2, NEK11, NRG3, OPHN1, OXGR1, PAGE4, PARD3,
PDE4D, PDS5B, PDZRN4, PKIID1, PLCL1, PPP2R2B, PRKAG2, PROS1, PRPF8, PTPN22, PTPRD, PTPRN2, PXDNL, RAPGEF2, ROBO2,
SCAPER, SEMA6D, SLC24A3, SLC25A12, SLCO3A1, SPRED2, STAG2, STXBP5L, SUSDl, SYNE2, TCHH, TEK, THOP1, THSD7B, TLN2,
TNNI3K, TRHDE, TSC22D1, TUSC3, UGGT2, ULK4, UTRN, UVRAG, VPS13B, WWTR1, XKR4, XYLT1, YTHDC2, ZFPM2
>32
ABCA10, ABCA12, ABCA13, ABCA4, ABCB1, ABCC6, ACADS, ACADSB, ACOT7, ACSL4, ACSS2, ADAM23, ADCY8, AGBL4, AGK, AGXT,
AHI1, AKAP9, ALDH5A1, ALG8, ALOX5, ANK3, ANKRD30A, ANKRD50, ANKS1B, ANO3, ATRX, BBS5, BBS9, BEND7, BMPR2, BNC2,
BRAF, C12orf42, C4orf22, CADM2, CASP8, CASR, CCDC178, CCDC7, CCSER1, CCT8, CD22, CD2AP, CDH10, CDH12, CDH13, CDH2, CES3,
CFH, CNBD1, CNTLN, CNTN5, CNTNAP2, COL19A1, CPA4, CPNE4, CPNE8, CRHR1, CTNNA3, CTNND2, CUBN, CWC27, CYBB,
CYP1A1, CYP3A5, DAB1, DCC, DISC1, DLGAP2, DNAHII11, DNAII14, DNAJC13, DNM3, DOCK4, DYM, DYSF, EGR2, EN1, EPIIA7, EPIIX1,
EPHX2, ERBB3, ERBB4, EVC, EYS, FAM122C, FAT4, FEM1C, FGF14, FOXN3, FOXP2, FRAS1, FSTL4, FUT8, GABRA2, GABRB1,
GABRB3, GABRG2, GIF, GMPR, GPC5, GRID1, GRID2, GRIK2, GRM4, GRM5, GRM8, GTDC1, HCN1, HIVEP1, HMCN1, HMGCR, HMGN5,
HSD17B4, IL1RAPL1, IL1RAPL2, IMMP2L, IQGAP2, ITGAM, ITGAX, ITPR2, JAZF1, KCNH8, KCTD16, KCTD8, KDM6A, KIF26B, KRT6B,
KRTAP9-4, LARS2, LDIIC, LDLRAP1, LIIX8, LINGO2, LRRK2, MAST4, MB21D2, MCCC1, MMP2, MTG1, MTUS2, NCOA3, NEK11, NPIIP1,
NPR3, NRG3, NSUN6, OCA2, OPHN1, OSBPL1A, OTOL1, OXGR1, PAGE4, PARD3, PDE4D, PDS5B, PDZRN4, PHACTR1, PKHD1, PLCL1,
PLOD2, PPP2R2B, PRKAG2, PROS1, PRPF8, PTPN22, PTPRD, PTPRN2, PXDNL, RAPGEF2, RAPSN, RNF19A, ROBO2, SARDH, SCAPER,
SCG3, SEMA6D, SGCZ, SH3RF3, SLC24A3, SLC25A12, SLC6A3, SLCO3A1, SPAG17, SPRED2, SQSTM1, STAG2, STXBP5L, SUSD1, SYNE2,
TCIIII, TDRKII, TEK, TENM2, TFEC, TIIOP1, TIISD7B, TLN2, TMC1, TNNI3K, TRIIDE, TRIM36, TSC22D1, TUSC3, UGGT2, ULK4,
UTRN, UVRAG, VAV3, VAX1, VCAM1, VPS13B, WDPCP, WWTR1, XK, XKR4, YTHDC2, ZFPM2, ZNF608, ZPLD1
>33
ABCA10, ABCA12, ABCA13, ABCA4, ABCB1, ABCC6, ACADS, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BNC2, BRAF, C12orf42, C4orf22, CASP8, CASR, CCDC7, CDH10, CDH12,
CDH2, CFH, CNTN5, CNTNAP2, COL19A1, CTNNA3, CTNND2, CUBN, CYBB, CYP1A1, CYP3A5, DCC, DISC1, DLGAP2, DNAH11,
DNAH14, DNAJC13, DNM3, DOCK4, DYM, DYSF, EGR2, EPHA7, EPHX1, ERBB3, ERBB4, EVC, EYS, FAT4, FOXP2, FUT8, GABRA2,
GABRB1, GABRG2, GIF, GMPR, GR1D1, GRM4, GRM5, GRM8, GTDC1, HCN1, HIVEP1, HMCN1, HMGN5, IL1RAPL1, IMMP2L, ITGAM,
ITGAX, ITPR2, KCNH8, KDM6A, KRT6B, LHX8, LINGO2, LRRK2, MAST4, MCCC1, MTG1, MTUS2, NCOA3, NEK11, NRG3, OPHN1,
OSBPL1A, OXGR1, PAGE4, PARD3, PDE4D, PDS5B, PDZRN4, PKHD1, PLCL1, PPP2R2B, PRKAG2, PROS1, PRPF8, PTPN22, PTPRD,
PTPRN2, PXDNL, RAPGEF2, ROBO2, SCAPER, SEMA6D, SLC24A3, SLC25A12, SLCO3A1, SPRED2, STAG2, STXBP5L, SUSD1, SYNE2,
TCHH, TEK, TENM2, THOP1, THSD7B, TLN2, TNN13K, TRHDE, TRIM36, TSC22D1, TUSC3, UGGT2, ULK4, UTRN, UVRAG, VAV3,
VPS13B, WDPCP, WWTR1, XKR4, YTHDC2, ZFPM2, ZPLD1
>34
ABCA10, ABCA12, ABCA13, ABCA4, ABCB1, ABCC6, ABHD3, ACADS, ACADSB, ACOT7, ACSL4, ACSS2, ADAM23, ADAMTS6, ADCY8,
ADH1C, AGBL4, AGK, AGXT, AH11, AKAP9, ALDH3A2, ALDH5A1, ALG8, ALOX5, ANK3, ANKRD30A, ANKRD50, ANKS1B, ANO3,
ARHGAP24, ARHGEF4, ATRX, BBS5, BBS9, BEND7, BMPR2, BNC2, BRAF, C12orf42, C4orf22, CADM2, CASP8, CASR, CCDC105,
CCDC178, CCDC7, CCSER1, CCT8, CD22, CD28, CD2AP, CDH10, CDH12, CDH13, CDH2, CENPK, CES3, CFH, CNBD1, CNTLN, CNTN5,
CNTNAP2, COL19A1, CPA4, CPNE4, CPNE8, CPXCR1, CRHR1, CRYM, CTNNA3, CTNND2, CTSB, CUBN, CWC27, CYBB, CYP19A1,
CYP1A1, CYP3A5, DAB1, DCC, DDIT4, DISC1, DLD, DLGAP2, DMRTA1, DNAH11, DNAH14, DNAJC13, DNM3, DOCK4, DYM, DYSF,
EGR2, EMB, EN1, EPHA7, EPHX1, EPHX2, ERBB3, ERBB4, ESRRG, EVC, EYS, FAF1, FAM122C, FAM172A, FAM184A, FANCI, FAT4,
FEM1C, FGF14, FOXN3, FOXP2, FRAS1, FSTL4, FUT8, GABRA2, GABRB1, GABRB3, GABRG2, GALNT2, GIF, GMPR, GPC5, GPR6,
GRID1, GRID2, GRIK2, GRM4, GRM5, GRM8, GTDC1, GYS2, HCN1, HGSNAT, HIVEP1, HMCN1, HMGCR, HMGN5, HSD17B4, HYOU1,
IL1RAPL1, IL1RAPL2, IMMP2L, IQGAP2, ITGA8, ITGAM, ITGAX, ITPR2, JAZF1, KCNE1, KCNH8, KCTD16, KCTD8, KDM6A, KIF26B,
KLHL4, KRT6B, KRTAP9-4, KRTAP9-9, LALBA, LARS2, LDHC, LDLRAP1, LHX8, LINGO2, LIPG, LRRK2, MASP2, MAST4, MB21D2,
MCCC1, MERTK, MMP2, MTG1, MTUS2, MYRIP, NCOA3, NEK11, NELL1, NMT2, NPHP1, NPR3, NRG3, NSUN3, NSUN6, OCA2, OPHN1,
OSBPL1A, OTOL1, OXGR1, PAGE4, PARD3, PDE11A, PDE4D, PDS5B, PDZRN4, PHACTR1, PKHD1, PLCL1, PLOD2, PLXDC2, PPP2R2B,
PPT1, PRKAG2, PRMT6, PROS1, PRPF8, PTPN12, PTPN22, PTPRD, PTPRN2, PXDNL, RAB28, RAPGEF2, RAPSN, RHAG, RNF180,
RNF19A, ROBO2, RPL11, SARDH, SCAPER, SCG3, SEMA6D, SGCZ, SH3RF3, SIL1, SLC22A20, SLC24A3, SLC25A12, SLC37A2, SLC6A3,
SLCO3A1, SMOC2, SPAG17, SPRED2, SQSTM1, STAG2, STAM, STXBP5L, SUSD1, SYK, SYNE2, TCHH, TDRKH, TEK, TENM2, TFEC,
THOP1, THSD7B, TLN2, TMC1, TNFSF11, TNNI3K, TPST1, TRHDE, TRIM36, TRIM5, TSC22D1, TTC12, TUSC3, UBE2D2, UGGT2, ULK4,
UTRN, UVRAG, VAV3, VAX1, VCAM1, VPS13B, VWC2, WDPCP, WDR27, WWTR1, XK, XKR4, XYLT1, YTHDC2, ZBTB49, ZFPM2, ZNF608,
ZPLD1
>35
ABCA10, ABCA12, ABCA13, ABCA4, ABCB1, ABCC6, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BNC2, BRAF, C12orf42, C4orf22, CASP8, CASR, CCDC7, CDH10, CDH12,
CDH13, CDH2, CFH, CNTN5, CNTNAP2, COL19A1, CTNNA3, CTNND2, CTSB, CUBN, CYBB, CYP19A1, CYP1A1, CYP3A5, DCC, DISC1,
DLGAP2, DNAH11, DNAH14, DNAJC13, DOCK4, DYM, DYSF, EPHA7, ERBB3, ERBB4, ESRRG, EYS, FAT4, FOXP2, FUT8, GABRA2,
GABRB1, GABRG2, GIF, GMPR, GRM4, GRM5, GRM8, GTDC1, HCN1, HMCN1, HMGN5, HYAL1, IL1RAPL1, IMMP2L, ITGAM, ITGAX,
ITPR2, KCNH8, KDM6A, KRT6B, LDHC, LHX8, LINGO2, LRRK2, MAST4, MB21D2, MMP2, MTG1, MTUS2, NEK11, NRG3, OPHN1,
OXGR1, PAGE4, PARD3, PDE4D, PDS5B, PDZRN4, PKHD1, PLCL1, PPP2R2B, PRKAG2, PROS1, PRPF8, PTPN22, PTPRD, PTPRN2,
PXDNL, RAPGEF2, ROBO2, SCAPER, SEMA6D, SLC24A3, SLC25A12, SLCO3A1, SPRED2, STAG2, STXBP5L, SUSD1, SYNE2, TCHH, TEK,
THOP1, THSD7B, TLN2, TNNI3K, TRHDE, TSC22D1, TUSC3, UGGT2, ULK4, UTRN, UVRAG, VPS13B, WWTR1, XKR4, XYLT1, YTHDC2,
ZFPM2
>36
ABCA10, ABCA12, ABCA13, ABCA4, ABCB1, ABCC6, ACADS, ACADSB, ACOT7, ACSL4, ACSS2, ADAM23, ADCY8, AGBL4, AGK, AGXT,
AHI1, AKAP9, ALDH5A1, ALG8, ALOX5, ANK3, ANKRD30A, ANKRD50, ANKS1B, ANO3, ATRX, BBS5, BBS9, BEND7, BMPR2, BNC2,
BRAF, C12orf42, C4orf22, CADM2, CASP8, CASR, CCDC178, CCDC7, CCSER1, CCT8, CD22, CD2AP, CDH10, CDH12, CDH13, CDH2, CES3,
CFH, CNBD1, CNTLN, CNTN5, CNTNAP2, COL19A1, CPA4, CPNE4, CPNE8, CRHR1, CTNNA3, CTNND2, CTSB, CUBN, CWC27, CYBB,
CYP19A1, CYP1A1, CYP3A5, DAB1, DCC, DISC1, DLGAP2, DNAH11, DNAH14, DNAJC13, DNM3, DOCK4, DYM, DYSF, EGR2, EN1,
EPHA7, EPHX1, EPHX2, ERBB3, ERBB4, EVC, EYS, FAM122C, FAT4, FEM1C, FGF14, FOXN3, FOXP2, MAS1, FSTL4, FUT8, GABRA2,
GABRB1, GABRB3, GABRG2, GIF, GMPR, GPC5, GRID1, GRID2, GRIK2, GRM4, GRM5, GRM8, GTDC1, HCN1, HIVEP1, HMCN1, HMGCR,
HMGN5, HSD17B4, IL1RAPL1, IL1RAPL2, IMMP2L, IQGAP2, ITGAM, ITGAX, ITPR2, JAZF1, KCNH8, KCTD16, KCTD8, KDM6A, KIF26B,
KRT6B, KRTAP9-4, LARS2, LDHC, LDLRAP1, LHX8, LINGO2, LRRK2, MAST4, MB21D2, MCCC1, MMP2, MTG1, MTUS2, NCOA3, NEK11,
NPHP1, NPR3, NRG3, NSUN6, OCA2, OPHN1, OSBPL1A, OTOL1, OXGR1, PAGE4, PARD3, PDE4D, PDS5B, PDZRN4, PHACTR1, PKHD1,
PLCL1, PLOD2, PPP2R2B, PRKAG2, PROS1, PRPF8, PTPN22, PTPRD, PTPRN2, PXDNL, RAPGEF2, RAPSN, RNF19A, ROBO2, SARDH,
SCAPER, SCG3, SEMA6D, SGCZ, SH3RF3, SLC24A3, SLC25A12, SLC6A3, SLCO3A1, SPAG17, SPRED2, SQSTM1, STAG2, STXBP5L,
SUSD1, SYNE2, TCHH, TDRKH, TEK, TENM2, TFEC, THOP1, THSD7B, TLN2, TMC1, TNNI3K, TRHDE, TRIM36, TSC22D1, TUSC3,
UGGT2, ULK4, UTRN, UVRAG, VAV3, VAX1, VCAM1, VPS13B, WDPCP, WWTR1, XK, XKR4, YTHDC2, ZFPM2, ZNF608, ZPLD1
>37
ABCA10, ABCA12, ABCA13, ABCA4, ABCB1, ABCC6, ACADS, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BMPR2, BNC2, BRAF, C12orf42, C4orf22, CASP8, CASR, CCDC105, CCDC7,
CDH10, CDH12, CDH13, CDH2, CFH, CNTN5, CNTNAP2, COL19A1, CPNE8, CTNNA3, CTNND2, CTSB, CUBN, CYBB, CYP19A1, CYP1A1,
CYP3A5, DCC, DDIT4, DISC1, DLGAP2, DNAH11, DNAH14, DNAJC13, DNM3, DOCK4, DYM, DYSF, EGR2, EPHA7, EPHX1, ERBB3,
ERBB4, ESRRG, EVC, EYS, FAM122C, FAM184A, FAT4, FOXP2, FRAS1, FUT8, GABRA2, GABRB1, GABRB3, GABRG2, GIF, GMPR,
GRID1, GRM4, GRM5, GRM8, GTDC1, HCN1, HIVEP1, HMCN1, HMGN5, HSD17B4, ICOS, IL1RAPL1, IMMP2L, ITGAM, ITGAX, ITPR2,
KCNH8, KCTD8, KDM6A, KLHL3, KRT6B, LDHC, LHX8, LINGO2, LRRK2, MAST4, MB21D2, MCCC1, MMP2, MTG1, MTUS2, NCOA3,
NEK11, NFKB1, NRG3, OCA2, OPHN1, OSBPL1A, OXGR1, PAGE4, PARD3, PDE11A, PDE4D, PDPN, PDS5B, PDZRN4, PKHD1, PLCL1,
PPP2R2B, PRKAG2, PROS1, PRPF8, PTPN12, PTPN22, PTPRD, PTPRN2, PXDNL, RAPGEF2, ROBO2, RPL11, SCAPER, SEMA6D, SH3RF3,
SLC24A3, SLC25A12, SLCO3A1, SPRED2, STAG2, STXBP5L, SUSD1, SYNE2, TCHH, TEK, TENM2, THOP1, THSD7B, TLN2, TNFSF11,
TNNI3K, TRHDE, TRIM36, TSC22D1, TUSC3, UGGT2, ULK4, UTRN, UVRAG, VAV3, VCAM1, VPS13B, WDPCP, WWTR1, XKR4, XYLT1,
YTHDC2, ZFPM2, ZPLD1
>38
ABCA10, ABCA12, ABCA13, ABCA4, ABCB1, ABCC6, ACADS, ACADSB, ACOT7, ACSL4, ACSS2, ADAM23, AGBL4, AGK, AGXT, AKAP9,
ALDH5A1, ALOX5, ANK3, ANKRD30A, ANKRD50, ANKS1B, ANO3, ATRX, BBS5, BBS9, BNC2, BRAF, C12orf42, C4orf22, CADM2, CASP8,
CASR, CCDC105, CCDC7, CDH10, CDH12, CDH13, CDH2, CFH, CNTN5, CNTNAP2, COL19A1, CPNE8, CTNNA3, CTNND2, CTSB, CUBN,
CWC27, CYBB, CYP19A1, CYP1A1, CYP3A5, DCC, DDIT4, DISC1, DLGAP2, DNAH11, DNAH14, DNAJC13, DNM3, DOCK4, DYM, DYSF,
EGR2, EPHA7, EPHX1, ERBB3, ERBB4, ESRRG, EVC, EYS, FAM122C, FAM184A, FAT4, FOXP2, FRAS1, FUT8, GABRA2, GABRB1,
GABRB3, GABRG2, GIF, GMPR, GRID1, GRM4, GRM5, GRM8, GTDC1, IICN1, IIIVEP1, IIMCN1, IIMGCR, IIMGN5, IISD17B4, ICOS,
IL1RAPL1, IMMP2L, ITGAM, ITGAX, ITPR2, KCNH8, KCTD8, KDM6A, KRT6B, KRTAP9-4, LHX8, LINGO2, LRRK2, MAST4, MB21D2,
MCCC1, MMP2, MTG1, MTUS2, NCOA3, NEK11, NMT2, NRG3, OCA2, OPHN1, OSBPL1A, OXGR1, PAGE4, PARD3, PDE4D, PDPN,
PDS5B, PDZRN4, PKHD1, PLCL1, PLOD2, PPP2R2B, PRKAG2, PROS1, PRPF8, PTPN12, PTPN22, PTPRD, PTPRN2, PXDNL, RAPGEF2,
ROBO2, RPL11, SCAPER, SCG3, SEMAGD, SII3RF3, SLC24A3, SLC25A12, SLCO3A1, SPRED2, STAG2, STXBP5L, SUSD1, SYNE2, TCIIII,
TEK, TENM2, THOP1, THSD7B, TLN2, TNFSF11, TNNI3K, TRHDE, TRIM36, TSC22D1, TUSC3, UGGT2, ULK4, UTRN, UVRAG, VAV3,
VCAM1, VPS13B, WDPCP, WWTR1, XK, XKR4, YTHDC2, ZFPM2, ZPLD1
>39
ABCA10, ABCA12, ABCA13, ABCA4, ABCB1, ABCC6, ACADS, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BNC2, BRAF, C12orf42, C4orf22, CASP8, CASR, CCDC7, CDH10, CDH12,
CDH13, CDH2, CFH, CNTN5, CNTNAP2, COL19A1, CTNNA3, CTNND2, CTSB, CUBN, CYBB, CYP19A1, CYP1A1, CYP3A5, DCC, DISC1,
DLGAP2, DNAH11, DNAH14, DNAJC13, DNM3, DOCK4, DYM, DYSF, EGR2, EPHA7, EPHX1, ERBB3, ERBB4, EVC, EYS, FAM122C, FAT4,
FOXP2, FUT8, GABRA2, GABRB1, GABRG2, GIF, GMPR, GRID1, GRM4, GRM5, GRM8, GTDC1, IICN1, IIIVEP1, IIMCN1, IIMGN5, ICOS,
IL1RAPL1, IMMP2L, ITGAM, ITGAX, ITPR2, KCNH8, KDM6A, KRT6B, LDHC, LHX8, LINGO2, LRRK2, MAST4, MB21D2, MCCC1, MMP2,
MTG1, MTUS2, NCOA3, NEK11, NRG3, OCA2, OPHN1, OSBPL1A, OXGR1, PAGE4, PARD3, PDE4D, PDS5B, PDZRN4, PKHD1, PLCL1,
PPP2R2B, PRKAG2, PROS1, PRPF8, PTPN22, PTPRD, PTPRN2, PXDNL, RAPGEF2, ROBO2, SCAPER, SEMA6D, SLC24A3, SLC25A12,
SLCO3A1, SPRED2, STAG2, STXBP5L, SUSD1, SYNE2, TCIIII, TEK, TENM2, TIIOP1, TIISD7B, TLN2, TNNI3K, TRIIDE, TRIM36,
TSC22D1, TUSC3, UGGT2, ULK4, UTRN, UVRAG, VAV3, VCAM1, VPS13B, WDPCP, WWTR1, XKR4, XYLT1, YTHDC2, ZFPM2, ZPLD1
>40
ABCA10, ABCA12, ABCA13, ABCA4, ABCB1, ABCC6, ACADS, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BMPR2, BNC2, BRAF, C12orf42, C4orf22, CASP8, CASR, CCDC105, CCDC7,
CDH10, CDH12, CDH13, CDH2, CFH, CNTN5, CNTNAP2, COL19A1, CPNE8, CTNNA3, CTNND2, CTSB, CUBN, CYBB, CYP19A1, CYP1A1,
CYP3A5, DCC, DDIT4, DISC1, DLGAP2, DNAH11, DNAH14, DNAJC13, DNM3, DOCK4, DYM, DYSF, EGR2, EPHA7, EPHX1, ERBB3,
ERBB4, ESRRG, EVC, EYS, FAM122C, FAM184A, FAT4, FOXP2, FRAS1, FUT8, GABRA2, GABRB1, GABRB3, GABRG2, GIF, GMPR,
GRID1, GRM4, GRM5, GRM8, GTDC1, HCN1, HIVEP1, HMCN1, HMGN5, HSD17B4, HYAL1, ICOS, IL1RAPL1, IMMP2L, ITGAM, ITGAX,
ITPR2, KCNH8, KCTD8, KDM6A, KLHL3, KRT6B, LDHC, LHX8, LINGO2, LRRK2, MAST4, MB21D2, MCCC1, MMP2, MTG1, MTUS2,
NCOA3, NEK11, NFKB1, NRG3, OCA2, OPHN1, OSBPL1A, OXGR1, PAGE4, PARD3, PDE11A, PDE4D, PDPN, PDS5B, PDZRN4, PKHD1,
PLCL1, PPP2R2B, PRKAG2, PROS1, PRPF8, PTPN12, PTPN22, PTPRD, PTPRN2, PXDNL, RAPGEF2, ROBO2, RPL11, SCAPER, SEMA6D,
SH3RF3, SLC24A3, SLC25A12, SLCO3A1, SPRED2, STAG2, STXBP5L, SUSD1, SYNE2, TCHH, TEK, TENM2, THOP1, THSD7B, TLN2,
TNFSF11, TNNI3K, TRHDE, TRIM36, TSC22D1, TUSC3, UGGT2, ULK4, UTRN, UVRAG, VAV3, VCAM1, VPS13B, WDPCP, WWTR1, XKR4,
XYLT1, YTHDC2, ZFPM2, ZPLD1
>41
ABCA10, ABCA12, ABCA13, ABCA4, ABCB1, ABCC6, ACADS, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BMPR2, BNC2, BRAF, C12orf42, C4orf22, CASP8, CASR, CCDC105, CCDC7,
CDH10, CDH12, CDH13, CDH2, CFH, CNTN5, CNTNAP2, COL19A1, CPNE8, CTNNA3, CTNND2, CTSB, CUBN, CYBB, CYP19A1, CYP1A1,
CYP3A5, DCC, DDIT4, DISC1, DLGAP2, DNAH11, DNAH14, DNAJC13, DNM3, DOCK4, DYM, DYSF, EGR2, EPHA7, EPHXI, ERBB3,
ERBB4, ESRRG, EVC, EYS, FAM122C, FAM184A, FAT4, FOXP2, FRAS1, FUT8, GABRA2, GABRB1, GABRB3, GABRG2, GIF, GMPR,
GRID1, GRM4, GRM5, GRM8, GTDC1, HCN1, HIVEP1, HMCN1, HMGN5, HSD17B4, HYAL1, IL1RAPL1, IMMP2L, ITGAM, ITGAX, ITPR2,
KCNH8, KCTD8, KDM6A, KLHL3, KRT6B, LDHC, LHX8, LINGO2, LRRK2, MAST4, MB21D2, MCCC1, MMP2, MTG1, MTUS2, NCOA3,
NEK11, NEKB1, NRG3, OCA2, OPHN1, OSBPL1A, OXGR1, PAGE4, PARD3, PDE11A, PDE4D, PDPN, PDS5B, PDZRN4, PKHD1, PLCL1,
PPP2R2B, PRKAG2, PROS1, PRPF8, PTPN12, PTPN22, PTPRD, PTPRN2, PXDNL, RAPGEF2, ROBO2, RPL11, SCAPER, SEMA6D, SH3RF3,
SLC24A3, SLC25A12, SLCO3A1, SPRED2, STAG2, STXBP5L, SUSD1, SYNE2, TCHH, TEK, TENM2, THOP1, THSD7B, TLN2, TNFSF11,
TNNI3K, TRHDE, TRIM36, TSC22D1, TUSC3, UGGT2, ULK4, UTRN, UVRAG, VAV3, VCAM1, VPS13B, WDPCP, WWTR1, XKR4, XYLT1,
YTHDC2, ZFPM2, ZPLD1
>42
ABCA10, ABCA12, ABCA13, ABCA4, ABCB1, ABCC6, ACADS, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BMPR2, BNC2, BRAF, C12orf42, C4orf22, CASP8, CASR, CCDC105, CCDC7,
CDH10, CDH12, CDH13, CDH2, CFH, CNTN5, CNTNAP2, COL19A1, CPNE8, CTNNA3, CTNND2, CTSB, CUBN, CWC27, CYBB, CYP19A1,
CYP1A1, CYP3A5, DCC, DDIT4, DISC1, DLGAP2, DNAH11, DNAH14, DNAJC13, DNM3, DOCK4, DYM, DYSF, EGR2, EPHA7, EPHX1,
ERBB3, ERBB4, ESRRG, EVC, EYS, FAM122C, FAM184A, FAT4, FOXP2, FRAS1, FSTL4, FUT8, GABRA2, GABRB1, GABRB3, GABRG2,
GIF, GMPR, GRID1, GRIK2, GRM4, GRM5, GRM8, GTDC1, HCN1, HIVEP1, HMCN1, HMGN5, HSD17B4, ICOS, IL1RAPL1, IL1RAPL2,
IMMP2L, ITGAM, ITGAX, ITPR2, KCNH8, KCTD8, KDM6A, KLHL3, KRT6B, LDHC, LHX8, LINGO2, LRRK2, MAST4, MB21D2, MCCC1,
MMP2, MTG1, MTUS2, NCOA3, NEK11, NEKB1, NMT2, NRG3, OCA2, OPHN1, OSBPL1A, OXGR1, PAGE4, PARD3, PDE11A, PDE4D,
PDS5B, PDZRN4, PKHD1, PLCL1, PPP2R2B, PRKAG2, PROS1, PRPF8, PTPN12, PTPN22, PTPRD, PTPRN2, PXDNL, RAPGEF2, ROBO2,
RPL11, SCAPER, SEMA6D, SH3RF3, SLC24A3, SLC25A12, SLCO3A1, SPRED2, STAG2, STXBP5L, SUSD1, SYNE2, TCHH, TEK, TENM2,
THOP1, THSD7B, TLN2, TNFSF11, TNNI3K, TRHDE, TRIM36, TSC22D1, TUSC3, UGGT2, ULK4, UTRN, UVRAG, VAV3, VCAM1, VPS13B,
WDPCP, WWTR1, XKR4, XYLT1, YTHDC2, ZFPM2, ZPLD1
>43
ABCA10, ABCA12, ABCA13, ABCA4, ABCB1, ABCC6, ACADS, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BNC2, BRAF, C12orf42, C4orf22, CASP8, CASR, CCDC7, CDH10, CDH12,
CDH13, CDH2, CFH, CNTN5, CNTNAP2, COL19A1, CTNNA3, CTNND2, CTSB, CUBN, CYBB, CYP19A1, CYP1A1, CYP3A5, DCC, DISC1,
DLGAP2, DNAH11, DNAH14, DNAJC13, DNM3, DOCK4, DYM, DYSF, EGR2, EPHA7, EPHX1, ERBB3, ERBB4, ESRRG, EVC, EYS,
FAM122C, FAT4, FOXP2, FUT8, GABRA2, GABRB1, GABRG2, GIF, GMPR, GRID1, GRM4, GRM5, GRM8, GTDC1, HCN1, HIVEP1,
HMCN1, HMGN5, HYAL1, ICOS, IL1RAPL1, IMMP2L, ITGAM, ITGAX, ITPR2, KCNH8, KDM6A, KRT6B, LDHC, LHX8, LINGO2, LRRK2,
MAST4, MB21D2, MCCC1, MMP2, MTG1, MTUS2, NCOA3, NEK11, NRG3, OCA2, OPHN1, OSBPL1A, OXGR1, PAGE4, PARD3, PDE4D,
PDS5B, PDZRN4, PKHD1, PLCL1, PPP2R2B, PRKAG2, PROS1, PRPF8, PTPN22, PTPRD, PTPRN2, PXDNL, RAPGEF2, ROBO2, SCAPER,
SEMA6D, SLC24A3, SLC25A12, SLCO3A1, SPRED2, STAG2, STXBP5L, SUSD1, SYNE2, TCHH, TEK, TENM2, THOP1, THSD7B, TLN2,
TNNI3K, TRHDE, TRIM36, TSC22D1, TUSC3, UGGT2, ULK4, UTRN, UVRAG, VAV3, VCAM1, VPS13B, WDPCP, WWTR1, XKR4, XYLT1,
YTHDC2, ZFPM2, ZPLD1
>44
ABCA10, ABCA12, ABCA13, ABCA4, ABCB1, ABCC6, ACADS, ACADSB, ACSL4, ACSS2, ADAM23, AGBL4, AGK, AKAP9, ALOX5, ANK3,
ANKRD30A, ANKRD50, ANKS1B, ANO3, ATRX, BBS5, BBS9, BNC2, BRAF, C12orf42, C4orf22, CASP8, CASR, CCDC7, CDH10, CDH12,
CDH13, CDH2, CFH, CNTN5, CNTNAP2, COL19A1, CTNNA3, CTNND2, CTSB, CUBN, CYBB, CYP19A1, CYP1A1, CYP3A5, DCC, DISC1,
DLGAP2, DNAH11, DNAH14, DNAJC13, DNM3, DOCK4, DYM, DYSF, EGR2, EPHA7, EPHX1, ERBB3, ERBB4, ESRRG, EVC, EYS,
FAM122C, FAT4, FOXP2, FUT8, GABRA2, GABRB1, GABRG2, GIF, GMPR, GRID1, GRM4, GRM5, GRM8, GTDC1, HCN1, HIVEP1,
HMCN1, HMGN5, HYAL1, IL1RAPL1, IMMP2L, ITGAM, ITGAX, ITPR2, KCNH8, KDM6A, KRT6B, LDHC, LHX8, LINGO2, LRRK2, MAST4,
MB21D2, MCCC1, MMP2, MTG1, MTUS2, NCOA3, NEK11, NRG3, OCA2, OPHN1, OSBPL1A, OXGR1, PAGE4, PARD3, PDE4D, PDS5B,
PDZRN4, PKHD1, PLCL1, PPP2R2B, PRKAG2, PROS1, PRPF8, PTPN22, PTPRD, PTPRN2, PXDNL, RAPGEF2, ROBO2, SCAPER, SEMA6D,
SLC24A3, SLC25A12, SLCO3A1, SPRED2, STAG2, STXBP5L, SUSD1, SYNE2, TCHH, TEK, TENM2, THOP1, THSD7B, TLN2, TNNI3K,
TRHDE, TRIM36, TSC22D1, TUSC3, UGGT2, ULK4, UTRN, UVRAG, VAV3, VCAM1, VPS13B, WDPCP, WWTR1, XKR4, XYLT1, YTHDC2,
ZFPM2, ZPLD1
>45
ABCA12, ACADS, ADAM23, ADCY8, AKAP9, ALOX5, ANKRD30A, ANKS1B, ANO3, ATRX, BBS9, BMPR2, BRAF, CADM2, CASP8, CASR,
CCDC105, CCDC7, CDH10, CDH12, CDH2, CFH, CNTLN, CNTN5, CNTNAP2, COL19A1, CPNE8, CRHR1, CRYM, CUBN, CYP19A1,
CYP3A5, DCC, DDIT4, DISC1, DLD, DNAH11, DNAH14, DNAJC13, DNM3, DOCK4, DYSF, EMB, EPHA7, ERBB3, ERBB4, ESRRG, EVC,
EYS, FAF1, FAM184A, FANCI, FAT4, FOXP2, FRAS1, FUT8, GABRA2, GABRB1, GABRB3, GALNT2, GLRA2, GRID2, GRIK2, GRM4, GRM5,
GYS2, HCN1, HIVEP1, HMCN1, HSD17B4, HYOU1, IL1RAPL1, ITGAX, ITPR2, KCTD8, KIF26B, KLHL3, KRTAP9-4, LRRK2, MAST4,
MCCC1, MMP2, MTUS2, NCOA3, NFKB1, NPHP1, NSUN6, OCA2, OPHN1, OTOL1, PDE11A, PDE4D, PDS5B, PHACTR1, PKHD1, PLCL1,
PLOD2, PLXDC2, PPARGC1A, PTPN12, PTPRD, PTPRN2, PXDNL, RAPGEF2, RHAG, ROBO2, RPL11, SCAPER, SH3RF3, SLC22A20,
SLC37A2, SMOC2, STAG2, SYK, SYNE2, TCHH, TEK, TENM2, TFEC, THSD7B, TLN2, TNFSF11, TNNI3K, TRHDE, TRIM36, ULK4,
UTRN, VAV3, VCAM1, XK, ZBTB49, ZFPM2
>46
ABCA10, ABCA12, ABCA13, ABCA4, ABCB1, ABCC6, ACADS, ACADSB, ACOT7, ACSL4, ACSS2, ADAM23, AGBL4, AGK, AGXT, AKAP9,
ALDH5A1, ALOX5, ANK3, ANKRD30A, ANKRD50, ANKS1B, ANO3, ATRX, BBS5, BBS9, BMPR2, BNC2, BRAF, C12orf42, C4orf22, CADM2,
CASP8, CASR, CCDC105, CCDC7, CDH10, CDH12, CDH13, CDH2, CFH, CNTN5, CNTNAP2, COL19A1, CPNE8, CTNNA3, CTNND2, CTSB,
CUBN, CWC27, CYBB, CYP19A1, CYP1A1, CYP3A5, DCC, DDIT4, DISC1, DLGAP2, DNAII11, DNAII14, DNAJC13, DNM3, DOCK4, DYM,
DYSF, EGR2, EPHA7, EPHX1, ERBB3, ERBB4, ESRRG, EVC, EYS, FAM122C, FAM184A, FAT4, FOXP2, FRAS1, FUT8, GABRA2, GABRB1,
GABRB3, GABRG2, GIF, GMPR, GRID1, GRM4, GRM5, GRM8, GTDC1, HCN1, HIVEP1, HMCN1, HMGCR, HMGN5, HSD17B4, ICOS,
IL1RAPL1, IMMP2L, ITGAM, ITGAX, ITPR2, KCNH8, KCTD8, KDM6A, KLHL3, KRT6B, KRTAP9-4, LDHC, LHX8, LINGO2, LRRK2,
MAST4, MB21D2, MCCC1, MMP2, MTG1, MTUS2, NCOA3, NEK11, NFKB1, NMT2, NRG3, OCA2, OPIIN1, OSBPL1A, OXGR1, PAGE4,
PARD3, PDE11A, PDE4D, PDPN, PDS5B, PDZRN4, PKHD1, PLCL1, PLOD2, PPP2R2B, PRKAG2, PROS1, PRPF8, PTPN12, PTPN22,
PTPRD, PTPRN2, PXDNL, RAPGEF2, ROBO2, RPL11, SCAPER, SCG3, SEMA6D, SH3RF3, SLC24A3, SLC25A12, SLCO3A1, SPRED2,
STAG2, STXBP5L, SUSD1, SYNE2, TCHH, TEK, TENM2, THOP1, THSD7B, TLN2, TNFSF11, TNNI3K, TRHDE, TRIM36, TSC22D1, TUSC3,
UGGT2, ULK4, UTRN, UVRAG, VAV3, VCAM1, VPS13B, WDPCP, WWTR1, XK, XKR4, XYLT1, YTIIDC2, ZFPM2, ZPLD1
>47
ABCA10, ABCA12, ABCA13, ABCB1, ACOT7, ACSL4, ADAM23, ADCY8, AGBL4, AGXT, AHI1, AKAP9, ALG8, ANK3, ANKRD30A,
ANKRD50, ANO3, ATRX, BEND7, BMPR2, BNC2, BRAF, C4orf22, CASP8, CASR, CCDC178, CCDC7, CCSER1, CCT8, CD22, CD2AP,
CDII10, CDII12, CDII13, CDII2, CES3, CFII, CNBD1, CNTLN, CNTN5, CNTNAP2, CPA4, CPNE4, CPNE8, CRIIR1, CTNNA3, CTNND2,
CUBN, CWC27, CXCL13, DAB1, DCC, DLGAP2, DNAH11, DNAH14, DNAJC13, DNM3, DOCK4, DYM, DYSF, EGR2, EN1, EPHA7, EPHX1,
EPHX2, ERBB3, ERBB4, EVC, EYS, FAT4, FEM1C, FGF14, FOXN3, FOXP2, FRAS1, FSTL4, GABRB3, GPC5, GRID2, GRIK2, GRM5,
GRM8, HCN1, HMCN1, HMGN5, IL1RAPL2, IQGAP2, ITGA8, ITGAX, JAZF1, KCNH8, KCTD16, KCTD8, KIF26B, KRT6B, LARS2, LDHC,
LDLRAP1, LINGO2, LRRK2, MAST4, MB21D2, MMP2, MTG1, MTUS2, MYRIP, NCOA3, NPIIP1, NPR3, NRG3, NSUN6, OCA2, OPIIN1,
OSBPL1A, OTOL1, PDPN, PDS5B, PDZRN4, PEX5, PHACTR1, PKHD1, PLOD2, PPP2R2B, PTPRN2, PXDNL, RAPGEF2, RAPSN, RNF19A,
ROBO2, SARDH, SCAPER, SEMA6D, SGCZ, SH3RF3, SLC24A3, SLC6A3, SLCO3A1, SPAG17, SPRED2, SQSTM1, STAG2, STXBP5L, SYNE2,
TCHH, TDRKH, TEK, TENM2, TFEC, THSD7B, TMC1, TNN13K, TRHDE, TSC22D1, TUSC3, UGGT2, ULK4, UTRN, VAX1, VCAM1,
VPS13B, WDPCP, WWTR1, YTHDC2, ZNF608
>48
ABCA10, ABCA12, ABCA13, ABCB1, ACSL4, ADAM23, ADCY8, AGBL4, AGXT, AHI1, AKAP9, ALG8, ANK3, ANKRD30A, ANKRD50,
ANO3, ATRX, BEND7, BMPK2, BNC2, BRAF, C4orf22, CASP8, CASR, CCDC178, CCDC7, CCSER1, CCT8, CD22, CD2AP, CDH10,
CDH12, CDH13, CDH2, CES3, CFH, CNBD1, CNTLN, CNTN5, CNTNAP2, CPA4, CPNE4, CPNE8, CRHR1, CTNNA3, CTNND2, CUBN,
CWC27, DAB1, DLGAP2, DNAH11, DNAH14, DNAJC13, DNM3, DOCK4, DYM, DYSF, EGR2, EN1, EPHA7, EPHX1, EPHX2, ERBB3,
ERBB4, EVC, EYS, FAT4, FEM1C, FGF14, FOXN3, FOXP2, FRAS1, FSTL4, GABRB3, GPC5, GRID2, GRIK2, GRM5, GRM8, HCN1,
HMCN1, HMGN5, IL1RAPL2, 1QGAP2, ITGAX, JAZF1, KCNH8, KCTD16, KCTD8, KIF26B, KKT6B, LARS2, LDHC, LDLRAP1, L1NGO2,
LRRK2, MAST4, MB21D2, MTG1, MTUS2, MYRIP, NCOA3, NPHP1, NPR3, NRG3, NSUN6, OCA2, OPHN1, OSBPL1A, OTOL1, PDS5B,
PDZRN4, PHACTR1, PKHD1, PLOD2, PPP2R2B, PTPRN2, PXDNL, RAPGEF2, RAPSN, RNF19A, ROBO2, SARDH, SCAPER, SEMA6D,
SGCZ, SH3RF3, SLC24A3, SLC6A3, SLCO3A1, SPAG17, SPRED2, SQSTM1, STXBP5L, SYNE2, TCHH, TDRKH, TEK, TENM2, TFEC,
THSD7B, TMC1, TNNI3K, TRHDE, TSC22D1, TUSC3, UGGT2, ULK4, UTRN, VAX1, VCAM1, VPS13B, WDPCP, WWTR1, YTHDC2,
ZNF608
>49
ABCA10, ABCA13, ABCB1, ADCY8, AGBL4, ANK3, ANO3, BEND7, BNC2, BRAF, CASR, CCDC178, CCT8, CDH10, CDH12, CDH2, CFH,
CNBD1, CNTLN, CNTN5, CNTNAP2, CPNE4, CRHR1, CTNND2, CUBN, DAB1, DLGAP2, DNAJC13, DOCK4, DYSF, EGR2, EPHA7, ERBB3,
EKBB4, EVC, EYS, FAT4, FSTL4, GABRB3, GRID2, GKM5, HMCN1, KCNH8, KCTD16, LINGO2, LRRK2, MAST4, MTUS2, MYRIP,
NCOA3, NPHP1, NPR3, OPHN1, PDS5B, PKHD1, PXDNL, SARDH, SEMA6D, SGCZ, SLC24A3, SLC6A3, SPRED2, STXBP5L, SYNE2, TCHH,
TDRKH, TENM2, THSD7B, TNNI3K, TRHDE, TUSC3, ULK4, UTRN, VAX1
>50
ABCA10, ABCA12, ABCA13, ABCB1, ADCY8, AGBL4, AGXT, AKAP9, ANK3, ANO3, BEND7, BMPR2, BNC2, BRAF, CASP8, CASR,
CCDC178, CCDC7, CCT8, CDH10, CDH12, CDH13, CDH2, CFH, CNBD1, CNTLN, CNTN5, CNTNAP2, CPNE4, CRHR1, CTNND2, CUBN,
CXCL13, DAB1, DCC, DLGAP2, DNAJC13, DOCK4, DYSF, EGR2, EPHA7, ERBB3, ERBB4, EVC, EYS, FAT4, FSTL4, GABRB3, GRID2,
GRIK2, GRM5, HMCN1, HMGCR, ITGA8, KCNH8, KCTD16, LINGO2, LRRK2, MAST4, MMP2, MTUS2, MYRIP, NCOA3, NFKB1, NPHP1,
NPR3, OPHN1, PDPN, PDS5B, PKHD1, PLOD2, PXDNL, ROBO2, SARDH, SEMA6D, SGCZ, SLC24A3, SLC6A3, SPRED2, STXBP5L, SYNE2,
TCHH, TDRKH, TENM2, THSD7B, TNNI3K, TRHDE, TUSC3, ULK4, UTRN, UVRAG, VAV3, VAX1
>51
ABCA13, ABCA4, ALDH5A1, ANK3, ANKRD30A, ANKS1B, BBS9, CADM2, CCSER1, CDH2, CLN3, CNTN5, CNTNAP2, COL19A1, CPNE4,
CTNND2, CUBN, DCC, DLGAP2, DNAH14, DOCK4, ERBB4, EYS, FAT4, FUT8, GABRB3, GRM8, GTDC1, GYS2, HCN1, ITGA8, JAZF1,
KIF26B, MAST4, NELL1, OCA2, PDZRN4, PKHD1, PPARGC1A, PROS1, PXDNL, ROBO2, SCG3, SGCZ, SOD1, STXBP5L, SYK, THOP1,
THSD7B, TLN2, TRHDE, VCAM1, WDR27, XKR4
>52
ABCA10, ABCA12, ABCA13, ABCB1, ADCY8, AGBL4, AGXT, AKAP9, ANK3, ANO3, BEND7, BMPR2, BNC2, BRAF, CASP8, CASR,
CCDC178, CCDC7, CCT8, CDH10, CDH12, CDH2, CFH, CNBD1, CNTLN, CNTN5, CNTNAP2, CPNE4, CRHR1, CTNND2, CUBN, DAB1,
DCC, DLGAP2, DNAJC13, DOCK4, DYSF, EGR2, EPHA7, ERBB3, ERBB4, EVC, EYS, FAT4, FSTL4, GABRB3, GRID2, GRIK2, GRM5,
HMCN1, HMGCR, KCNH8, KCTD16, LINGO2, LRRK2, MAST4, MTUS2, MYRIP, NCOA3, NFKB1, NPHP1, NPR3, OPHN1, PDPN, PDS5B,
PKHD1, PXDNL, SARDH, SEMA6D, SGCZ, SLC24A3, SLC6A3, SPRED2, STXBP5L, SYNE2, TCHH, TDRKH, TENM2, THSD7B, TNNI3K,
TRHDE, TUSC3, ULK4, UTRN, UVRAG, VAV3, VAX1
>53
ABCA12, ADAM23, ADCY8, AKAP9, ANKRD30A, ANKS1B, ANO3, ATRX, BBS9, BRAF, CADM2, CASR, CCDC7, CDH10, CDH12, CDH2,
CNTLN, CNTN5, CNTNAP2, COL19A1, CRHR1, CRYM, CUBN, DISC1, DLD, DNAH11, DNAH14, DNAJC13, DNM3, DOCK4, DYSF, EMB,
EPHA7, ERBB3, ERBB4, ESRRG, EVC, EYS, FAF1, FANCI, FAT4, FRAS1, GABRA2, GABRB1, GABRB3, GALNT2, GRID2, GRIK2, GRM4,
GRM5, GYS2, HCN1, HIVEP1, HMCN1, HYOU1, IL1RAPL1, ITPR2, KIF26B, KRTAP9-4, LRRK2, MAST4, MCCC1, MMP2, MTUS2, NCOA3,
NPHP1, NSUN6, OCA2, OPHN1, OTOL1, PDE4D, PDS5B, PHACTR1, PKHD1, PLCL1, PLOD2, PLXDC2, PTPRD, PXDNL, RAPGEF2, RHAG,
ROBO2, SCAPER, SH3RF3, SLC22A20, SLC37A2, SMOC2, STAG2, SYK, SYNE2, TENM2, TFEC, THSD7B, TLN2, TNNI3K, TRIM36,
UTRN, WWTR1, ZBTB49, ZFPM2
>54
ABCA10, ABCA13, ABCB1, ADCY8, AGBL4, ANK3, ANO3, BEND7, BNC2, BRAF, CASP8, CASR, CCDC178, CCT8, CDH10, CDH12, CDH2,
CFH, CNBD1, CNTLN, CNTN5, CNTNAP2, CPNE4, CRHR1, CTNND2, CUBN, DAB1, DCC, DLGAP2, DNAJC13, DOCK4, DYSF, EGR2,
EPHA7, ERBB3, ERBB4, EVC, EYS, FAT4, FSTL4, GABRB3, GRID2, GRM5, HMCN1, KCNH8, KCTD16, LINGO2, LRRK2, MAST4, MTUS2,
MYRIP, NCOA3, NPHP1, NPR3, OPHN1, PDS5B, PKHD1, PXDNL, SARDH, SEMA6D, SGCZ, SLC24A3, SLC6A3, SPRED2, STXBP5L,
SYNE2, TCHH, TDRKH, TENM2, THSD7B, TNNI3K, TRHDE, TUSC3, ULK4, UTRN, VAX1
>55
ABCA12, ADAM23, ADCY8, AKAP9, ANKRD30A, ANKS1B, ANO3, ATRX, BBS9, BRAF, CADM2, CASR, CCDC7, CDH10, CDH12, CNTLN,
CNTN5, CNTNAP2, COL19A1, CRHR1, CRYM, CUBN, DISC1, DLD, DNAH11, DNAH14, DNAJC13, DNM3, DOCK4, DYSF, EMB, EPHA7,
ERBB3, ERBB4, ESRRG, EVC, EYS, FAF1, FANCI, FAT4, FRAS1, GABRA2, GABRB1, GABRB3, GALNT2, GRID2, GRIK2, GRM4, GRM5,
GYS2, HCN1, HIVEP1, HMCN1, HYOU1, IL1RAPL1, ITPR2, KIF26B, KRTAP9-4, LRRK2, MAST4, MCCC1, MTUS2, NPHP1, NSUN6, OCA2,
OPHN1, OTOL1, PDE4D, PDS5B, PHACTR1, PKHD1, PLCL1, PLOD2, PLXDC2, PTPRD, PXDNL, RAPGEF2, RHAG, ROBO2, SCAPER,
SH3RF3, SLC22A20, SLC37A2, SMOC2, STAG2, SYK, SYNE2, TENM2, TFEC, THSD7B, TLN2, TNNI3K, TRIM36, UTRN, ZBTB49, ZFPM2
>56
ABCA12, ADAM23, ADCY8, AKAP9, ANKRD30A, ANKS1B, ANO3, ATRX, BBS9, BRAF, CADM2, CASR, CCDC178, CCDC7, CDH10, CDH12,
CDH2, CNTLN, CNTN5, CNTNAP2, COL19A1, CRHR1, CRYM, CTSB, CUBN, DISC1, DLD, DNAH11, DNAH14, DNAJC13, DNM3, DOCK4,
DYM, DYSF, EMB, EPHA7, ERBB3, ERBB4, ESRRG, EVC, EYS, FAF1, FANCI, FAT4, FRAS1, GABRA2, GABRB1, GABRB3, GALNT2,
GLRA2, GRID2, GRIK2, GRM4, GRM5, GYS2, HCN1, HIVEP1, HMCN1, HYOU1, IL1RAPL1, ITPR2, KDM6A, KIF26B, KRTAP9-4, KRTAP9-
8, LRRK2, MAST4, MCCC1, MTUS2, NPHP1, NSUN6, OCA2, OPHN1, OTOL1, PDE4D, PDS5B, PHACTR1, PKHD1, PLCL1, PLOD2,
PLXDC2, PTPRD, PXDNL, RAPGEF2, RHAG, ROBO2, SCAPER, SH3RF3, SLC22A20, SLC37A2, SMOC2, SPAG17, STAG2, SYK, SYNE2,
TENM2, TFEC, THSD7B, TLN2, TNNI3K, TRIM36, ULK4, UTRN, VAV3, ZBTB49, ZFPM2
>57
ABCA13, ABCA4, ALDH5A1, ANK3, ANKRD30A, ANKS1B, BBS9, BRAF, CADM2, CCSER1, CDH2, CLN3, CNTN5, CNTNAP2, COL19A1,
CPNE4, CTNND2, CUBN, DCC, DLGAP2, DNAH14, DOCK4, ERBB4, EYS, FAT4, FUT8, GABRB3, GRM8, GTDC1, GYS2, HCN1, ITGA8,
JAZF1, KIF26B, MAST4, NELL1, OCA2, PDZRN4, PKHD1, PPARGC1A, PROS1, PXDNL, ROBO2, SCG3, SGCZ, SOD1, STXBP5L, SYK,
THOP1, THSD7B, TLN2, TRHDE, VCAM1, WDR27, XKR4
>58
ABCA10, ABCA12, ABCA13, ABCB1, ADCY8, AGXT, AKAP9, ANK3, BEND7, BMPR2, BNC2, BRAF, CASP8, CASR, CCDC178, CCDC7,
CCT8, CDH12, CFH, CNBD1, CNTLN, CNTN5, CNTNAP2, CPNE4, CRHR1, CTNND2, CUBN, DLGAP2, DNAJC13, DOCK4, DYSF, EGR2,
ERBB3, ERBB4, EVC, FAT4, FSTL4, GABRB3, GRID2, GRIK2, GRM5, HMCN1, HMGCR, KCNH8, KCTD16, LINGO2, LRRK2, MAST4,
MMP2, MTUS2, NCOA3, NFKB1, NPIIP1, NPR3, OPIIN1, PDPN, PDS5B, PKIID1, PXDNL, SARDII, SGCZ, SLC24A3, SLC6A3, SPRED2,
STXBP5L, SYNE2, TCHH, TDRKH, TENM2, TNNI3K, TUSC3, ULK4, UTRN, UVRAG, VAV3, VAX1
>59
ABCA10, ABCA12, ABCA13, ABCB1, ADCY8, AGXT, AKAP9, ANK3, BEND7, BMPR2, BNC2, BRAF, CASP8, CASR, CCDC178, CCDC7,
CCT8, CDII12, CFII, CNBD1, CNTLN, CNTN5, CNTNAP2, CPNE4, CRIIR1, CTNND2, CUBN, DLGAP2, DNAJC13, DOCK4, DYSF, EGR2,
ERBB3, ERBB4, EVC, FAT4, FSTL4, GABRB3, GRID2, GRIK2, GRM5, HMCN1, HMGCR, KCNH8, KCTD16, LINGO2, LRRK2, MAST4,
MTUS2, NCOA3, NFKB1, NPHP1, NPR3, OPHN1, PDPN, PDS5B, PKHD1, PXDNL, SARDH, SGCZ, SLC24A3, SLC6A3, SPRED2, STXBP5L,
SYNE2, TCHH, TDRKH, TENM2, TNNI3K, TUSC3, ULK4, UTRN, UVRAG, VAV3, VAX1
>60
ABCA13, ABCB1, ACSS2, ADAM23, ADH1C, ANKRD30A, ANKS1B, ATRX, BMPR2, BRAF, C12orf42, CASP8, CDH10, CDH12, CDH13,
CLN3, CNTN5, CNTNAP2, CPXCR1, CTNND2, CUBN, CXCL13, CYP19A1, CYP1A1, CYP3A5, DCC, DYSF, EPHX1, ERBB3, ERBB4,
FRAS1, FUT8, GABRA2, GABRB1, GABRB3, GABRG2, GMPR, GPC5, GRID2, GRM5, GRM8, HCN1, HPGD, HSD17B4, IL15, ITGAX,
KCNH8, KCTD16, KDM6A, KRT6B, LRRK2, MASP2, MB21D2, MMP2, NCOA3, NEK11, NFKB1, PDE4D, PIIACTR1, RIIAG, ROBO2,
SLC6A3, SQSTM1, STAG2, SYNE2, TCHH, TEK, TNFSF11, TRHDE, ULK4, UTRN, VCAM1
>61
ABCA13, ABCB1, ACSS2, ADAM23, ADH1C, ANKRD30A, ANKS1B, ATRX, BMPR2, BRAF, C12orf42, CASP8, CDH10, CDH12, CDH13,
CLN3, CNTN5, CNTNAP2, CPXCR1, CTNND2, CUBN, CXCL13, CYP19A1, CYP1A1, CYP3A5, DCC, DYSF, EPIIX1, ERBB3, ERBB4,
FRAS1, FUT8, GABRA2, GABRB1, GABRB3, GABRG2, GMPR, GPC5, GRID2, GRM5, GRM8, HCN1, HPGD, HSD17B4, IL15, ITGAX,
KCNH8, KCTD16, KDM6A, KRT6B, LMO1, LRRK2, MASP2, MB21D2, MMP2, NCOA3, NEK11, NFKB1, PDE4D, PHACTR1, RHAG,
ROBO2, SLC6A3, SQSTM1, STAG2, SYNE2, TCHH, TEK, TNFSF11, TRHDE, ULK4, UTRN, VCAM1
>62
ABCA10, ABCA12, ABCA13, ABCA4, ABCB1, ACADS, ACOT7, ACSL4, ACSS2, ADAM23, ADAMTS6, AGBL4, AGK, AGXT, AHI1, AKAP9,
ALG8, ALOX5, ANK3, ANKRD30A, ANKRD50, ANKS1B, ATRX, BMPR2, BNC2, BRAF, C12orf42, C4orf22, CAMKMT, CAPN3, CASP8,
CASR, CCDC178, CCDC7, CCSER1, CD22, CD28, CD2AP, CDH10, CDH13, CDH2, C`ES3, CFH, CNBD1, CNTLN, CNTN5, CNTNAP2, CPA4,
CPNE8, CTNNA3, CTNND2, CTSB, CUBN, CWC27, CYP19A1, CYP1A1, CYP3A5, DAB1, DCC, DDIT4, DNAH11, DNAH14, DNAJC13, DNM3,
DOCK4, DYM, DYSF, EGR2, EN1, EPHX1, EPHX2, ERBB3, ERBB4, ESRRG, FAT4, FEM1C, FGF14, FOXN3, FOXP2, FRAS1, FSTL4,
GABRB3, GPC5, GRIK2, GRM5, GRM8, GSTM2, HCN1, HMCN1, HMGCR, HMGN5, IL15, IL1RAPL1, IL1RAPL2, IQGAP2, ITGAM, ITGAX,
JAZF1, KCNH8, KCTD16, KCTD8, KDM6A, KIF26B, KRT6B, KRTAP9-9, LARS2, LDHC, LDLRAP1, LINGO2, LMO1, LRRK2, MASP2,
MAST4, MB21D2, MERTK, mir-548, MMP2, MTG1, MTUS2, NCOA3, NPHP1, NRG3, NSUN6, NUDT13, OCA2, OSBPL1A, OTOL1, PDE4D,
PDPN, PDS5B, PDZRN4, PEX5, PHACTR1, phosphate, PLOD2, PPP2R2B, PRKAG2, PTPN12, PTPN22, PTPRD, PTPRN2, PXDNL, RAPGEF2,
RAPSN, RNF19A, ROBO2, SCAPER, SH3RF3, SIL1, SLC24A3, SLCO3A1, SOD1, SPAG17, SQSTM1, STAG2, SYNE2, TCHH, TEK, TFEC,
THSD7B, TMC1, TNFSF11, TNN13K, TSC22D1, UGGT2, UTRN, VAX1, VCAM1, VPS13B, WDPCP, WWTR1, YTHDC2, ZNF608
>63
ABCA13, ABCB1, ADAM23, ADH1C, ANKRD30A, ANKS1B, ATRX, BMPR2, BRAF, C12orf42, CASP8, CDH10, CDH12, CDH13, CLN3,
CNTN5, CNTNAP2, CPXCR1, CTNND2, CUBN, CXCL13, CYP19A1, CYP1A1, DCC, DYSF, ERBB3, ERBB4, FRAS1, FUT8, GABRA2,
GABRB1, GABRB3, GABRG2, GMPR, GPC5, GR1D2, GRM5, GRM8, HCN1, HPGD, HSD17B4, IL15, ITGAX, KCNH8, KCTD16, KDM6A,
KRT6B, LRRK2, MASP2, MB21D2, MMP2, NCOA3, NEK11, NFKB1, PDE4D, PHACTR1, RHAG, ROBO2, SLC6A3, SQSTM1, STAG2, SYNE2,
TCHH, TEK, TNFSF11, TRHDE, ULK4, UTRN, VCAM1
>64
ABCA12, ADAM23, ADCY8, AKAP9, ANKRD30A, ANKS1B, ANO3, ATRX, BBS9, BRAF, CADM2, CASR, CCDC7, CDH10, CDH12, CDH2,
CNTLN, CNTN5, CNTNAP2, COL19A1, CRHR1, CRYM, CUBN, DISC1, DLD, DNAH11, DNAH14, DNAJC13, DNM3, DOCK4, DYSF, EMB,
EPHA7, ERBB3, ERBB4, ESRRG, EVC, EYS, FAF1, FANCI, FAT4, FRAS1, GABRA2, GABRB1, GABRB3, GALNT2, GLRA2, GRID2, GRIK2,
GRM4, GRM5, GYS2, HCN1, HIVEP1, HMCN1, HYOU1, IL1RAPL1, ITPR2, KIF26B, KRTAP9-4, LRRK2, MAST4, MCCC1, MTUS2, NPHP1,
NSUN6, OCA2, OPHN1, OTOL1, PDE4D, PDS5B, PHACTR1, PKHD1, PLCL1, PLOD2, PLXDC2, PTPRD, PXDNL, RAPGEF2, RHAG, ROBO2,
SCAPER, SH3RF3, SLC22A20, SLC37A2, SMOC2, STAG2, SYK, SYNE2, TENM2, TFEC, THSD7B, TLN2, TNNI3K, TRIM36, UTRN,
ZBTB49, ZFPM2
>65
ABCB1, ADCY8, AHI1, ALOX5, BRAF, CASR, CCDC7, CD28, CFH, CTNNA3, CTSB, CXCL13, CYBB, CYP19A1, CYP1A1, DCC, DYM,
EPHX2, EVC, FAF1, GABRA2, GABRB1, GABRB3, GABRG2, GALNT2, GRM8, HMGCR, HPGD, HTR3B, ICOS, IL15, LRRK2, MASP2,
MERTK, MMP2, NELL1, NFKB1, NKAIN2, OCA2, PDE11A, PDE4D, PPP2R2B, PTPN22, PTPRN2, RPL11, SIL1, SLC6A3, SYK, TBC1D32,
TENM4, THOP1, TNFSF11, TPST1, VAV3, VCAM1, YTHDC2
>66
ABCA13, ABCB1, ADAM23, ADH1C, ANKRD30A, ANKS1B, ATRX, BRAF, C12orf42, CASP8, CDH10, CDH12, CDH13, CLN3, CNTN5,
CNTNAP2, CPXCR1, CTNND2, CUBN, CYP19A1, DCC, DYSF, ERBB3, ERBB4, FRAS1, FUT8, GABRA2, GABRB1, GABRB3, GABRG2,
GMPR, GPC5, GRID2, GRM5, GRM8, HCN1, HPGD, HSD17B4, ITGAX, KCNH8, KCTD16, KDM6A, KRT6B, LRRK2, MASP2, MB21D2,
MMP2, NCOA3, NEK11, NFKB1, PHACTR1, RHAG, ROBO2, SLC6A3, STAG2, SYNE2, TCHH, TEK, TNFSF11, TRHDE, ULK4
>67
ABCA10, ABCA13, ADCY8, ANK3, BEND7, BNC2, BRAF, CASR, CCT8, CDH12, CFH, CNBD1, CNTLN, CNTN5, CNTNAP2, CPNE4,
CRHR1, CTNND2, CUBN, DLGAP2, DNAJC13, DYSF, EGR2, ERBB3, ERBB4, EVC, FAT4, FSTL4, GABRB3, GRID2, GRM5, HMCN1,
KCNH8, KCTD16, LINGO2, LRRK2, MAST4, MTUS2, NCOA3, NPHP1, NPR3, OPHN1, PKHD1, PXDNL, SARDH, SGCZ, SLC24A3, SLC6A3,
SPRED2, STXBP5L, SYNE2, TCHH, TDRKH, TENM2, TNNI3K, TUSC3, ULK4, UTRN, VAX1
>68
ABCA10, ABCA4, ABCB1, ABCC6, ACADS, adenosine triphosphate, AGXT, AHI1, ALDH5A1, ANK3, ARHGEF4, ATRX, BRAF, CASP8,
CCDC137, CCT8, CD22, CNTN5, CNTNAP2, CTNNA3, CTNND2, CYP19A1, CYP1A1, DISC1, DNAH14, EPHA7, EPHX2, ERBB3, ERBB4,
EYS, HTR3B, HYOU1, IL15, ITGAM, LRRK2, MASP2, MERTK, MMP2, MTUS2, NCOA3, NSUN3, OCA2, OSBPL1A, PDE4D, PDPN, PEX5,
PHACTR1, phosphate, PPARGC1A, PTPN12, PTPRN2, ROBO2, SEMA6D, SLC24A3, STAG2, STXBP5L, TCHH, TEK, TMC1, TNFSF11,
TNNI3K, TRIM5, VAV3, VPS13B, WDPCP, WDR27
>69
ABCA10, ABCA12, ABCA13, ABCB1, ACOT7, ACSL4, ADAM23, AGBL4, AGXT, AHI1, AKAP9, ALG8, ANK3, ANKRD30A, ANKRD50,
ATRX, BMPR2, BNC2, BRAF, C4orf22, CASP8, CASR, CCDC178, CCDC7, CCSER1, CD22, CD2AP, CDH10, CDH13, CDH2, CES3,
CNBD1, CNTLN, CNTN5, CNTNAP2, CPA4, CPNE8, CTNNA3, CTNND2, CUBN, CWC27, DAB1, DCC, DNAH11, DNAH14, DNM3,
DOCK4, DYM, DYSF, EGR2, EN1, EPHX1, EPHX2, ERBB3, ERBB4, FAT4, FEM1C, FGF14, FOXN3, FOXP2, FRAS1, FSTL4, GABRB3,
GPC5, GRIK2, GRM5, GRM8, HCN1, HMCN1, HMGN5, IL1RAPL2, IQGAP2, ITGAX, JAZF1, KCNH8, KCTD16, KCTD8, KIF26B, KRT6B,
LARS2, LDHC, LDLRAP1, LINGO2, LRRK2, MB21D2, MMP2, MTG1, MTUS2, NCOA3, NPHP1, NRG3, NSUN6, OCA2, OSBPL1A,
OTOL1, PDS5B, PDZRN4, PEX5, PHACTR1, PLOD2, PPP2R2B, PTPRN2, PXDNL, RAPGEF2, RAPSN, RNF19A, ROBO2, SCAPER,
SH3RF3, SLC24A3, SLCO3A1, SPAG17, SQSTM1, STAG2, SYNE2, TCHH, TEK, TFEC, THSD7B, TMC1, TNNI3K, TSC22D1, UGGT2,
UTRN, VAX1, VCAM1, VPS13B, WDPCP, WWTR1, YTHDC2, ZNF608
>70
ABCA10, ABCA13, ADCY8, ANK3, BEND7, BNC2, BRAF, CASP8, CASR, CCT8, CDH12, CFH, CNBD1, CNTLN, CNTN5, CNTNAP2, CPNE4,
CRHR1, CTNND2, CUBN, DLGAP2, DNAJC13, DYSF, EGR2, ERBB3, ERBB4, EVC, FAT4, FSTL4, GABRB3, GRID2, GRM5, HMCN1,
KCNH8, KCTD16, LINGO2, LRRK2, MAST4, MTUS2, NCOA3, NPHP1, NPR3, OPHN1, PKHD1, PXDNL, SARDH, SGCZ, SLC24A3, SLC6A3,
SPRED2, STXBP5L, SYNE2, TCHH, TDRKH, TENM2, TNNI3K, TUSC3, ULK4, UTRN, VAX1
>71
ABCA13, ABCB1, ADAM23, ADH1C, ANKRD30A, ANKS1B, ATRX, BRAF, C12orf42, CASP8, CDH10, CDH12, CDH13, CLN3, CNTN5,
CNTNAP2, CPXCR1, CTNND2, CUBN, CYP19A1, DCC, DYSF, ERBB3, ERBB4, FRAS1, FUT8, GABRA2, GABRB1, GABRB3, GABRG2,
GMPR, GPC5, GRID2, GRM5, GRM8, HCN1, HPGD, HSD17B4, ITGAX, KCNH8, KCTD16, KDM6A, KRT6B, LRRK2, MASP2, MB21D2,
MMP2, NCOA3, NEK11, NFKB1, PHACTR1, RHAG, ROBO2, SLC6A3, STAG2, SYNE2, TCHH, TEK, TNFSF11, TRHDE, ULK4, VCAM1
>72
ABCA10, ABCA12, ABCA13, ABCB1, ACOT7, ACSL4, ADAM23, AGBL4, AGXT, AHI1, AKAP9, ALG8, ANK3, ANKRD30A, ANKRD50,
ATRX, BMPR2, BNC2, BRAF, C4orf22, CASP8, CASR, CCDC178, CCDC7, CCSER1, CD22, CD2AP, CDH10, CDH13, CDH2, CES3,
CNBD1, CNTLN, CNTN5, CNTNAP2, CPA4, CPNE8, CTNNA3, CTNND2, CUBN, CWC27, DAB1, DCC, DNAH11, DNAH14, DNM3,
DOCK4, DYM, DYSF, EGR2, EN1, EPHX2, ERBB3, ERBB4, FAT4, FEM1C, FGF14, FOXN3, FOXP2, FRAS1, FSTL4, GABRB3, GPC5,
GRIK2, GRM5, GRM8, HCN1, HMCN1, HMGN5, IQGAP2, ITGAX, JAZF1, KCNH8, KCTD16, KCTD8, KIF26B, KRT6B, LARS2, LDHC,
LDLRAP1, LINGO2, LRRK2, MB21D2, MTG1, MTUS2, NPHP1, NRG3, NSUN6, OCA2, OSBPL1A, OTOL1, PDS5B, PDZRN4, PEX5,
PHACTR1, PLOD2, PPP2R2B, PTPRN2, PXDNL, RAPGEF2, RAPSN, RNF19A, ROBO2, SCAPER, SH3RF3, SLC24A3, SLCO3A1,
SPAG17, SQSTM1, SYNE2, TCHH, TEK, TFEC, TIISD7B, TMC1, TNNI3K, TSC22D1, UGGT2, UTRN, VAX1, VCAM1, VPS13B, WDPCP,
WWTR1, YTIIDC2, ZNF608
>73
ABCA10, ABCA4, ABCB1, ABCC6, ACADS, adenosine triphosphate, AGXT, AHI1, ALDH5A1, ANK3, ARHGEF4, ATRX, BRAF, CASP8,
CCDC137, CCT8, CD22, CFH, CNTN5, CNTNAP2, COL19A1, CTNNA3, CTNND2, CYP19A1, CYP1A1, DISC1, DNAH14, EPHA7, EPHX2,
ERBB3, ERBB4, ESRRG, EYS, IITR3B, IIYAL1, IIYOU1, IL15, ITGAM, LRRK2, MASP2, MERTK, MMP2, MTUS2, NCOA3, NSUN3, OCA2,
OSBPL1A, PDE4D, PDPN, PDS5B, PEX5, PHACTR1, phosphate, PPARGC1A, PRPF8, PTPN12, PTPRN2, ROBO2, SEMA6D, SLC24A3,
STAG2, STXBP5L, TCHH, TEK, TMC1, TNFSF11, TNNI3K, TRIM5, UTRN, VAV3, VPS13B, WDPCP, WDR27, ZFPM2
>74
ABCA10, ABCA12, ABCA13, ABCB1, ACSL4, ADAM23, AGBL4, AGXT, AIII1, AKAP9, ALG8, ANK3, ANKRD30A, ANKRD50, ATRX,
BMPR2, BNC2, BRAF, C4orf22, CASP8, CASR, CCDC178, CCDC7, CCSER1, CD22, CD2AP, CDH10, CDH13, CDH2, CESS, CNBD1,
CNTLN, CNTN5, CNTNAP2, CPA4, CPNE8, CTNNA3, CTNND2, CUBN, CWC27, DAB1, DCC, DNAH11, DNAH14, DNM3, DOCK4, DYM,
DYSF, EGR2, EN1, EPHX1, EPHX2, ERBB3, ERBB4, FAT4, FEM1C, FGF14, FOXN3, FOXP2, FRAS1, FSTL4, GABRB3, GPC5, GRIK2,
GRM5, GRM8, HCN1, IIMCN1, IIMGN5, IL1RAPL2, IQGAP2, ITGAX, JAZF1, KCNII8, KCTD16, KCTD8, KIF26B, KRT6B, LARS2, LDIIC,
LDLRAP1, LINGO2, LRRK2, MB21D2, MTG1, MTUS2, NCOA3, NPHP1, NRG3, NSUN6, OCA2, OSBPL1A, OTOL1, PDS5B, PDZRN4,
PHACTR1, PLOD2, PPP2R2B, PTPRN2, PXDNL, RAPGEF2, RAPSN, RNF19A, ROBO2, SCAPER, SH3RF3, SLC24A3, SLCO3A1,
SPAG17, SQSTM1, SYNE2, TCHH, TEK, TFEC, THSD7B, TMC1, TNNI3K, TSC22D1, UGGT2, UTRN, VAX1, VCAM1, VPS13B, WDPCP,
WWTR1, YTHDC2, ZNF608
>75
ABCA10, ABCA4, ABCB1, ABCC6, ACADS, AGXT, AHI1, ALDH5A1, ANK3, ARHGEF4, BRAF, CCDC137, CCT8, CD22, CNTNAP2, CTNNA3,
CTNND2, CYP1A1, DISC1, DNAH14, EPHA7, EPHX2, ERBB3, HTR3B, HYOU1, ITGAM, LRRK2, MERTK, NCOA3, NSUN3, OCA2,
OSBPL1A, PHACTR1, PPARGC1A, PTPN12, PTPKN2, ROBO2, SLC24A3, STXBP5L, TCHH, TMC1, TRIM5, VAV3, VPS13B, WDPCP, WDR27
>76
ABCA10, ABCA12, ABCA13, ABCB1, ACSL4, ADAM23, AGBL4, AGXT, AHI1, AKAP9, ALG8, ANK3, ANKRD30A, ANKRD50, ATRX,
BMPR2, BNC2, BRAF, C4orf22, CASP8, CASR, CCDC178, CCDC7, CCSER1, CD22, CD2AP, CDH10, CDH13, CDH2, CES3, CNBD1,
CNTLN, CNTN5, CNTNAP2, CPA4, CPNE8, CTNNA3, CTNND2, CUBN, CWC27, DAB1, DNAH11, DNAH14, DNM3, DOCK4, DYM,
DYSF, EGR2, EN1, EPHX2, ERBB3, ERBB4, FAT4, FEM1C, FGF14, FOXN3, FOXP2, FRAS1, FSTL4, GABRB3, GPC5, GRIK2, GRM5,
GRM8, HCN1, HMCN1, HMGN5, IQGAP2, ITGAX, JAZF1, KCNH8, KCTD16, KCTD8, KIF26B, KRT6B, LARS2, LDHC, LDLRAP1, LINGO2,
LRRK2, MB21D2, MTG1, MTUS2, NPHP1, NRG3, NSUN6, OCA2, OSBPL1A, OTOL1, PDS5B, PDZRN4, PHACTR1, PLOD2, PPP2R2B,
PTPRN2, PXDNL, RAPGEF2, RAPSN, KNF19A, KOBO2, SCAPER, SH3RF3, SLC24A3, SLCO3A1, SPAG17, SQSTM1, SYNE2, TCHH,
TEK, TFEC, THSD7B, TMC1, TNNI3K, TSC22D1, UGGT2, UTRN, VAX1, VCAM1, VPS13B, WDPCP, WWTR1, YTHDC2, ZNF608
>77
ABCA10, ABCA12, ABCA13, ABCB1, ACSL4, ADAM23, AGBL4, AGXT, AHI1, AKAP9, ALG8, ANK3, ANKRD30A, ANKRD50, ATRX,
BMPR2, BNC2, BRAF, C4orf22, CASP8, CASR, CCDC178, CCDC7, CCSER1, CD22, CD2AP, CDH10, CDH13, CDH2, CES3, CNBD1,
CNTLN, CNTN5, CNTNAP2, CPA4, CPNE8, CTNNA3, CTNND2, CUBN, CWC27, DAB1, DNAH11, DNAH14, DNM3, DOCK4, DYM,
DYSF, EGR2, EN1, EPHX1, EPHX2, ERBB3, ERBB4, FAT4, FEM1C, FGF14, FOXN3, FOXP2, FRAS1, FSTL4, GABRB3, GPC5, GRIK2,
GRM5, GRM8, HCN1, HMCN1, HMGN5, IL1RAPL2, IQGAP2, ITGAX, JAZF1, KCNH8, KCTD16, KCTD8, KIF26B, KRT6B, LARS2, LDHC,
LDLRAP1, LINGO2, LRRK2, MB21D2, MTG1, MTUS2, NCOA3, NPHP1, NRG3, NSUN6, OCA2, OSBPL1A, OTOL1, PDS5B, PDZRN4,
PHACTR1, PLOD2, PPP2R2B, PTPRN2, PXDNL, RAPGEF2, RAPSN, RNF19A, ROBO2, SCAPER, SH3RF3, SLC24A3, SLCO3A1,
SPAG17, SQSTM1, SYNE2, TCHH, TEK, TFEC, THSD7B, TMC1, TNNI3K, TSC22D1, UGGT2, UTRN, VAX1, VCAM1, VPS13B, WDPCP,
WWTR1, YTHDC2, ZNF608
>78
ABCA10, ABCA4, ABCB1, ABCC6, ACADS, adenosine triphosphate, AGXT, AHI1, ALDH5A1, ANK3, ARHGEF4, ATRX, BRAF, CASP8,
CCDC137, CCT8, CD22, CNTN5, CNTNAP2, CTNNA3, CTNND2, CYP19A1, CYP1A1, DISC1, DNAH14, EPHA7, EPHX2, ERBB3, ERBB4,
EYS, HTR3B, HYOU1, IL15, ITGAM, LRRK2, MASP2, MERTK, MMP2, MTUS2, NCOA3, NSUN3, OCA2, OSBPL1A, PDE4D, PDPN, PEX5,
PHACTR1, phosphate, PPARGC1A, PTPN12, PTPRN2, ROBO2, SEMA6D, SLC24A3, STAG2, STXBP5L, TCHH, TMC1, TNFSF11, TNNI3K,
TRIM5, VAV3, VPS13B, WDPCP, WDR27
>79
ABCA10, ABCA4, ABCB1, ABCC6, ACADS, adenosine triphosphate, AGXT, AHI1, ALDH5A1, ANK3, ARHGEF4, ATRX, BRAF, CASP8,
CCDC137, CCT8, CD22, CNTN5, CNTNAP2, CTNNA3, CTNND2, CYP19A1, CYP1A1, DISC1, DNAH14, EPHA7, EPHX2, ERBB3, ERBB4,
EYS, HTR3B, HYOU1, IL15, ITGAM, LRRK2, MASP2, MERTK, MMP2, MTUS2, NCOA3, NSUN3, OCA2, OSBPL1A, PDE4D, PDPN, PEX5,
PHACTR1, phosphate, PPARGC1A, PTPN12, PTPRN2, ROBO2, SEMA6D, SLC24A3, STAG2, STXBP5L, TCHH, TMC1, TNNI3K, TRIM5,
VAV3, VPS13B, WDPCP, WDR27
>80
ABCA10, ABCA12, ABCA13, ABCB1, ACOT7, ACSL4, ADAM23, AGBL4, AGXT, AHI1, AKAP9, ALG8, ANK3, ANKRD30A, ANKRD50,
ATRX, BMPR2, BNC2, BRAF, C4orf22, CASP8, CASR, CCDC178, CCDC7, CCSER1, CD22, CD2AP, CDH10, CDH13, CDH2, CES3,
CNBD1, CNTLN, CNTN5, CNTNAP2, CPA4, CPNE8, CTNNA3, CTNND2, CTSB, CUBN, CWC27, DAB1, DCC, DNAH11, DNAH14,
DNM3, DOCK4, DYM, DYSF, EGR2, EN1, EPHX2, ERBB3, ERBB4, FAT4, FEM1C, FGF14, FOXN3, FOXP2, FRAS1, FSTL4, GABRB3,
GPC5, GRIK2, GRM5, GRM8, HCN1, HMCN1, HMGN5, HPGD, IQGAP2, ITGAX, JAZF1, KCNH8, KCTD16, KCTD8, KIF26B, KRT6B,
LARS2, LDHC, LDLRAP1, LINGO2, LRRK2, MB21D2, MMP2, MTG1, MTUS2, NPHP1, NRG3, NSUN6, OCA2, OSBPL1A, OTOL1, PDPN,
PDS5B, PDZRN4, PEX5, PHACTR1, PLOD2, PPP2R2B, PTPN12, PTPRN2, PXDNL, RAPGEF2, RAPSN, RNF19A, ROBO2, SCAPER,
SH3RF3, SLC24A3, SLCO3A1, SPAG17, SQSTM1, SYNE2, TCHH, TEK, TFEC, THSD7B, TMC1, TNNI3K, TSC22D1, UGGT2, UTRN,
VAX1, VCAM1, VPS13B, WDPCP, WWTR1, YTHDC2, ZNF608
>81
ABCA10, ABCA12, ABCA13, ABCB1, ACADS, ACOT7, ACSL4, ADAM23, AGBL4, AGXT, AHI1, AKAP9, ALG8, ALOX5, ANK3, ANKRD30A,
ANKBD50, ATRX, BMPR2, BNC2, BRAF, C4orf22, CAPN3, CASP8, CASR, CCDC178, CCDC7, CCSER1, CD22, CD28, CD2AP, CDH10,
CDH13, CDH2, CES3, CFH, CNBD1, CNTLN, CNTN5, CNTNAP2, CPA4, CPNE8, CTNNA3, CTNND2, CTSB, CUBN, CWC27, DAB1, DCC,
DNAH11, DNAH14, DNM3, DOCK4, DYM, DYSF, EGR2, EN1, EPHX1, EPHX2, ERBB3, ERBB4, FAT4, FEM1C, FGF14, FOXN3, FOXP2,
FRAS1, FSTL4, GABRB3, GPC5, GRIK2, GRM5, GRM8, HCN1, HMCN1, HMGCR, HMGN5, HPGD, IL15, IL1RAPL2, IQGAP2, ITGAM,
ITGAX, JAZF1, KCNH8, KCTD16, KCTD8, KIF26B, KRT6B, LARS2, LDHC, LDLRAP1, LINGO2, LRRK2, MASP2, MB21D2, MERTK, mir-
548, MMP2, MTG1, MTUS2, NCOA3, NPHP1, NRG3, NSUN6, OCA2, OSBPL1A, OTOL1, PDE4D, PDPN, PDS5B, PDZRN4, PEX5, PHACTR1,
phosphate, PLOD2, PPP2R2B, PTPN12, PTPRN2, PXDNL, RAPGEF2, RAPSN, RNF19A, ROBO2, SCAPER, SH3RF3, SLC24A3, SLCO3A1,
SPAG17, SQSTM1, STAG2, SYNE2, TCHH, TEK, TFEC, THSD7B, TMC1, TNNI3K, TSC22D1, UGGT2, UTRN, VAX1, VCAM1, VPS13B,
WDPCP, WWTR1, YTHDC2, ZNF608
>82
ADCY8, adenosine triphosphate, ADGRB3, BRAF, CASR, CHRNA7, CLN3, CNTNAP2, CRHR1, CTNND2, CTSB, CYBB, CYP19A1, DCC,
DISC1, DMRTA1, DNM3, EGR2, EN1, EPHX2, ERBB4, GABRB3, GABRG2, GRID1, GRID2, GRIK2, GRM4, GRM5, GRM8, HCN1, HMGCR,
HSD17B4, IL15, ITGA8, ITPR2, KHT6B, LALBA, LHX8, LRRK2, NFKB1, NPR3, OPHN1, PDE11A, PDE4D, PPARGC1A, PPP1R14C, PPT1,
PTPRN2, RNF180, ROBO2, SLC6A3, SOD1, SQSTM1, TPST1, VCAM1
>83
ABCA12, ABCA4, ABCC6, ADGRV1, AHI1, ANK3, ASIC2, BARX1, BRAF, CFH, CHRNA7, CLN3, CNTNAP2, CTSB, CYP19A1, DAB1, DCC,
EGR2, EN1, EPHA7, ERBB3, ERBB4, FAT4, GABRB3, GRID2, ILIRAPL1, ITGA8, KRT6B, LHX8, MERTK, MMP2, NFKB1, NPHP1, NPR3,
OPHN1, PDS5B, PEX5, PPARGC1A, PPT1, PROS1, ROBO2, SC5D, SLC6A3, SOD1, TENM2, TNFSF11, ULK4, VAX1, WDPCP
>84
ABCA10, ABCA12, ABCA13, ABCB1, ACADS, ACOT7, ACSL4, ADAM23, AGBL4, AGXT, AHI1, AKAP9, ALG8, ALOX5, ANK3, ANKRD30A,
ANKRD50, ATRX, BMPR2, BNC2, BRAF, C4orf22, CAPN3, CASP8, CASR, CCDC178, CCDC7, CCSER1, CD22, CD28, CD2AP, CDH10,
CDH13, CDH2, CES3, CFH, CNBD1, CNTLN, CNTN5, CNTNAP2, CPA4, CPNE8, CTNNA3, CTNND2, CTSB, CUBN, CWC27, DAB1, DCC,
DNAH11, DNAH14, DNM3, DOCK4, DYM, DYSF, EGR2, EN1, EPHX1, EPHX2, ERBB3, ERBB4, FAT4, FEM1C, FGF14, FOXN3, FOXP2,
FRAS1, FSTL4, GABRB3, GPC5, GRIK2, GRM5, GRM8, HCN1, HMCN1, HMGCR, HMGN5, IL15, IL1RAPL2, IQGAP2, ITGAM, ITGAX,
JAZF1, KCNH8, KCTD16, KCTD8, KIF26B, KRT6B, LARS2, LDHC, LDLRAP1, LINGO2, LRRK2, MASP2, MB21D2, MERTK, mir-548, MMP2,
MTG1, MTUS2, NCOA3, NPHP1, NRG3, NSUN6, OCA2, OSBPL1A, OTOL1, PDE4D, PDS5B, PDZRN4, PEX5, PHACTR1, phosphate,
PLOD2, PPP2R2B, PTPN12, PTPRN2, PXDNL, RAPGEF2, RAPSN, RNF19A, ROBO2, SCAPER, SH3RF3, SLC24A3, SLCO3A1, SPAG17,
SQSTM1, STAG2, SYNE2, TCHH, TEK, TFEC, THSD7B, TMC1, TNNI3K, TSC22D1, UGGT2, UTRN, VAX1, VCAM1, VPS13B, WDPCP,
WWTR1, YTHDC2, ZNF608
>85
adenosine triphosphate, ADGRB3, AHI1, AKAP9, ANK3, ARHGAP24, ARHGEF4, BBS5, BBS9, BRAF, CD2AP, CDH13, CDH2, CNTNAP2,
CRHR1, CTNND2, CYP19A1, DAB1, DCC, DDAH1, DISC1, DNM3, EPHA7, EPHX2, ERBB4, FUT8, GRID2, GRM4, GRM5, ICOS, ILIRAPL1,
LRRK2, NFKB1, OPIIN1, PARD3, PDPN, PKIID1, PTPN20, RAPGEF2, RAPSN, RIT2, ROBO2, SOD1, SYK, TBC1D32, TENM4, TNFSF11,
ULK4, VAV3, WDPCP, WWTR1
>86
ADAMTS6, AHI1, CAPN3, CASP8, CD22, CD28, CFH, CNTNAP2, COL19A1, CPNE4, CTSB, CXCL13, CYBB, CYP19A1, DAB1, DDAH1,
DLGAP2, DYM, ERBB3, FGF14, FSTL4, GABRA2, GABRB1, GABRB3, GABRG2, GRIK2, IICN1, HMGCR, IIPGD, IITR3B, IIYAL1, ICOS,
IL15, ITGAM, ITGAX, JAZF1, KCNE1, KCTD16, LARS2, MERTK, NFKB1, NRG3, PDE11A, PDE4D, phosphate, PPP2R2B, PTPN22, PXDNL,
ROBO2, RTP4, SGCZ, SLC6A3, SOD1, SQSTM1, TEK, TNFSF11, TUSC3, VCAM1
>87
ABCA10, ABCA4, ABCB1, ABCC6, ACADS, AGXT, AIII1, ALDII5A1, ANK3, ARIIGEF4, BRAF, CCDC137, CCT8, CD22, CFII, CNTNAP2,
CTNNA3, CTNND2, CYP19A1, CYP1A1, DISC1, DNAH14, EPHA7, EPHX2, ERBB3, ERBB4, ESRRG, EYS, HTR3B, HYAL1, HYOU1, ITGAM,
LRRK2, MASP2, MERTK, MMP2, MTUS2, NCOA3, NSUN3, OCA2, OSBPL1A, PDS5B, PHACTR1, PPARGC1A, PRPF8, PTPN12, PTPRN2,
ROBO2, SEMA6D, SLC24A3, STXBP5L, TCHH, TMC1, TRIM5, UTRN, VAV3, VPS13B, WDPCP, WDR27
>88
ABCB1, ADAMTS6, ADCY8, AHI1, ALOX5, CASP8, CCDC7, CD22, CD28, CFH, CTNNA3, CTSB, CXCL13, CYBB, CYP19A1, DYM, EGR2,
EPHX2, ERBB3, EVC, FAF1, FGF14, FSTL4, GABEA2, GABRB1, GABRB3, GABRG2, GALNT2, HMGCR, HTR3B, ICOS, IL15, ITGAM,
ITGAX, LRRK2, MERTK, MMP2, NELL1, NFKB1, NRG3, OCA2, PDE11A, PDE4D, PPP2R2B, PTPN22, PTPRN2, PXDNL, RPL11, RTP4,
SGCZ, SIL1, SYK, TNFSF11, TPST1, VCAM1, YTIIDC2
>89
ABCA12, ABCA4, ABCC6, ADGRV1, AHI1, ANK3, ASIC2, BARX1, BRAF, CFH, CHRNA7, CLN3, CNTNAP2, CTSB, CYP19A1, DAB1, DCC,
EGR2, EN1, EPHA7, ERBB3, ERBB4, FAT4, GABRB3, GH1D1, GR1D2, ILIRAPLI, 1TGA8, KKT6B, LHX8, MERTK, MMP2, NFKB1, NPHP1,
NPR3, OPHN1, PDS5B, PEX5, PPARGC1A, PPT1, PROS1, ROBO2, SC5D, SLC6A3, SOD1, TENM2, TNFSF11, ULK4, VAX1, WDPCP
>90
ABCA10, ABCA4, ABCB1, ABCC6, ACADS, AGXT, AHI1, ALDH5A1, ANK3, ARHGEF4, BRAF, CCDC137, CCT8, CD22, CNTNAP2, CTNNA3,
CTNND2, CYP19A1, CYP1A1, DISC1, DNAH14, EPHA7, EPHX2, ERBB3, EYS, HTR3B, HYOL1, ITGAM, LRRK2, MASP2, MERTK, MTUS2,
NCOA3, NSUN3, OCA2, OSBPL1A, PHACTR1, PPARGC1A, PTPN12, PTPRN2, ROBO2, SEMA6D, SLC24A3, STXBP5L, TCHH, TMC1,
TRIM5, VAV3, VPS13B, WDPCP, WDR27
>91
ABCA10, ABCA12, ABCA13, ABCB1, ACOT7, ACSL4, ADAM23, AGBL4, AGXT, AH11, AKAP9, ALG8, ANK3, ANKRD30A, ANKRD50,
ATRX, BMPR2, BNC2, BRAF, C4orf22, CASP8, CASR, CCDC178, CCDC7, CCSER1, CD22, CD2AP, CDH10, CDH13, CDH2, CES3,
CNBD1, CNTLN, CNTN5, CNTNAP2, CPA4, CPNE8, CTNNA3, CTNND2, CTSB, CUBN, CWC27, DAB1, DCC, DNAH11, DNAH14,
DNM3, DOCK4, DYM, DYSF, EGR2, EN1, EPHX2, ERBB3, ERBB4, FAT4, FEM1C, FGF14, FOXN3, FOXP2, FRAS1, FSTL4, GABRB3,
GPC5, GRIK2, GRM5, GRM8, HCN1, HMCN1, HMGN5, 1QGAP2, ITGAX, JAZF1, KCNH8, KCTD16, KCTD8, KIF26B, KRT6B, LARS2,
LDHC, LDLRAP1, L1NGO2, LRRK2, MB21D2, MMP2, MTG1, MTUS2, NPHP1, NRG3, NSUN6, OCAS, OSBPL1A, OTOL1, PDS5B,
PDZRN4, PEX5, PHACTR1, PLOD2, PPP2R2B, PTPN12, PTPRN2, PXDNL, RAPGEF2, RAPSN, RNF19A, ROBO2, SCAPER, SH3RF3,
SLC24A3, SLCO3A1, SPAG17, SQSTM1, SYNE2, TCHH, TEK, TFEC, THSD7B, TMC1, TNNI3K, TSC22D1, UGGT2, UTRN, VAX1,
VCAM1, VPS13B, WDPCP, WWTR1, YTHDC2, ZNF608
>92
adenosine triphosphate, ADGRB3, ADGRL2, AHI1, ALOX5, ANK3, ARHGAP24, ASIC2, ATRX, BMPR2, BRAF, CASP8, CD28, CDH13, CDH2,
CHRNA7, CNTNAP2, CRHR1, CTNND2, CTSB, CYBB, CYP19A1, CYP1A1, DAB1, DCC, DDAH1, DDIT4, DISC1, DNM3, EGR2, EN1,
EPHA7, EPHX2, ERBB3, ERBB4, FOXP2, FUT8, GABRB3, GABRG2, GRID2, GRM4, GRM5, HCN1, HSD17B4, ICOS, IL15, IL1RAPL1,
IMMP2L, LHX8, LINGO2, LMO1, LRRK2, MERTK, MMP2, NCOA3, NFKB1, NRG3, OCA2, OPHN1, PARD3, PPARGC1A, PPT1, PROS1,
PTPN20, PTPN22, RAB28, RAPGEF2, RAPSN, RHAG, RILPL2, RIT2, RNASE10, ROBO2, SMOC2, SOD1, SQSTM1, STAM, SYK, TDRKH,
TEK, TENM4, TNFSF11, UTRN, UVRAG, VAV3, VCAM1, VWC2, ZFPM2
>93
ABCA10, ABCA4, ABCB1, ABCC6, ACADS, AGXT, AHI1, ALDH5A1, ANK3, ARHGEF4, BRAF, CCDC137, CCT8, CD22, CNTNAP2, CTNNA3,
CTNND2, CYP1A1, DISC1, DNAH14, EPHA7, EPHX2, ERBB3, EYS, HTR3B, HYOU1, ITGAM, LRRK2, MASP2, MERTK, MTUS2, NCOA3,
NSUN3, OCA2, OSBPL1A, PHACTR1, PPARGC1A, PTPN12, PTPRN2, ROBO2, SEMA6D, SLC24A3, STXBP5L, TCHH, TMC1, TRIM5, VAV3,
VPS13B, WDPCP, WDR27
>94
ABCB1, adenosine triphosphate, ALDH5A1, ANK3, C4orf22, CHRNA7, CNBD1, CRHR1, CYP19A1, CYP1A1, GABRA2, GABRB1, GABRB3,
GABRG2, GRID1, GRIK2, GRM4, GRM5, HMGCR, HTR3B, ITPR2, MCCC1, NEK11, NKAIN2, PDE11A, phosphate, SLC6A3, SYK, TENM4,
VPS13B
>95
ABCA4, AHI1, ANK3, ASIC2, BRAF, CDH2, CFH, CHRNA7, CLN3, CNTNAP2, CTSB, DAB1, DCC, DNM3, EGR2, EN1, EPHA7, ERBB3,
ERBB4, GABRB3, GRID1, GRID2, GRM5, IL1RAPL1, LHX8, LRRK2, MERTK, NFKB1, OPHN1, PARD3, PDS5B, PEX5, PPARGC1A, PPT1,
PROS1, RAPSN, ROBO2, SLC6A3, SOD1, SYNE2, TENM2, ULK4, UTRN, VAX1, XK
>96
ABCA4, AHI1, ANK3, ASIC2, BRAF, CFH, CHRNA7, CLN3, CNTNAP2, CTSB, DAB1, DCC, DNM3, EGR2, EN1, EPHA7, ERBB3, ERBB4,
GABRB3, GRID2, IL1RAPL1, LHX8, LRRK2, MERTK, NFKB1, OPHN1, PARD3, PDS5B, PEX5, PPARGC1A, PPT1, PROS1, RAPSN, ROBO2,
SLC6A3, SOD1, SYNE2, TENM2, ULK4, UTRN, VAX1, XK
>97
ABCA10, ABCA12, ABCA4, ACADS, ACSS2, ADAM23, ADAMTS6, AGK, AKAP9, ANKRD30A, ANKS1B, ANO3, ATRX, BMPR2, BRAF,
C12orf42, CAMKMT, CASP8, CCDC7, CD22, CDH10, CDH2, CFH, CNTLN, CNTNAP2, CTNND2, CUBN, CYP19A1, DNAH11, DNAH14,
DNAJC13, DNM3, DYSF, EGR2, EN1, EPHX1, ERBB3, ERBB4, ESRRG, EVC, FAM122C, FAT4, FRAS1, GRID1, GRIK2, GRM8, HIVEP1,
HMCN1, HMGN5, IL1RAPL1, ITGAX, KCNH8, KCTD16, KDM6A, KRT6B, KRTAP9-9, LINGO2, LRRK2, MAST4, MB21D2, MCCC1, MMP2,
MTUS2, NCOA3, NUDT13, OCA2, OPHN1, OSBPL1A, PDS5B, PHACTR1, PKHD1, PRKAG2, PTPN22, PTPRD, PTPRN2, PXDNL, ROBO2,
SLC25A12, SLCO3A1, SOD1, STAG2, TCHH, TENM2, THSD7B, TLN2, TRHDE, TRIM36, ULK4, UTRN, VAV3, VPS13B, WDPCP, WWTR1,
ZFPM2, ZNF608, ZPLD1
>98
ABCA12, ABCA4, ABCC6, ACADS, ACADSB, ADGRV1, AGK, AHI1, ANK3, BBS5, BBS9, CAPN3, CLN3, CYBB, DYSF, EGR2, EYS, FAT4,
GRID2, GRIK2, HMGCR, HPGD, HSD17B4, KCNE1, KLHL3, LARS2, LBR, LDLRAP1, MASP2, MCCC1, MERTK, MFSD8, MMP2, NPHP1,
OCA2, P3H1, PEX5, PKHD1, PLOD2, PROS1, RAB28, RHAG, SOD1, SQSTM1, TMC1, TNFSF11, TUSC3, VAX1, WDPCP, XYLT1
>99
adenosine triphosphate, ADGRB3, ADGRL2, ANK3, ASIC2, BRAF, CDH2, CHRNA7, CNTNAP2, CRHR1, CTNND2, CYP19A1, DAB1, DCC,
DDAH1, DISC1, DNM3, EGR2, EN1, EPHA7, ERBB3, ERBB4, FUT8, GABRB3, GABRG2, GRID2, GRM4, GRM5, ILIEAPL1, LHX8, LINGO2,
LRRK2, NFKB1, OPHN1, PARD3, PPT1, PTPN20, RAPGEF2, RAPSN, RIT2, ROBO2, SOD1, STAM, TENM4, UTRN
>100
ABCA12, ABCB1, ADAM23, ADGRV1, ADH1C, AHI1, AKAP9, ALDH5A1, ALOX5, ANK3, BARX1, BMPR2, BRAF, CASP8, CASR, CD22,
CD28, CD2AP, CDH2, CFH, CHRNA7, CLN3, COL19A1, CRHR1, CTNNA3, CTSB, CYBB, CYP1A1, DAB1, DCC, DDAH1, DLD, DNM3,
EGR2, EN1, ERBB3, ERBB4, ESRRG, FAT4, FUT8, GABRA2, GABRB3, GABRG2, GRID2, HMGCR, HPGD, HSD17B4, HYOU1, ICOS, IL15,
IQGAP2, ITGA8, ITGAM, ITPR2, KDM6A, KRT6B, LHX8, LMO1, LRRK2, MERTK, MMP2, NCOA3, NFKB1, NPR3, OPHN1, PARD3, PDPN,
PDS5B, PEX5, PLCL1, PPARGC1A, PPP1R14C, PPT1, PROS1, PRPF8, PTPN12, PTPRN2, RAPGEF2, RAPSN, ROBO2, SC5D, SLC6A3,
SOD1, SPRED2, SQSTM1, STAM, SYK, SYNE2, TEK, TNFSF11, ULK4, UTRN, VAV3, VAX1, VCAM1, WDPCP, ZFPM2
>101
ABCA10, ABCA12, ABCA13, ABCA4, ABCB1, ACADSB, AHI1, ANK3, ANKRD30A, ARHGAP24, BMPR2, BRAF, CCDC7, CD22, CD28,
CDH10, CFH, CNTLN, CNTN5, CNTNAP2, CTNNA3, CTNND2, CUBN, CWC27, CXCL13, CYP1A1, CYP3A5, DLEU1, DNAH11, DNAH14,
DYSF, EPHX1, EPHX2, FAT4, FRAS1, GRIK2, HIVEP1, HMCN1, IL15, KDM6A, LARS2, LMO1, LRRK2, MERTK, MMP2, NCOA3, NEK11,
NFKB1, OSBPL1A, OTOL1, PARD3, PDE4D, PHACTR1, PKHD1, PLOD2, PPP2R2B, PRPF8, PTPRD, PXDNL, RPL11, SGCZ, SLC22A20,
SLC24A3, SLC6A3, SPAG17, STAG2, STAM, SYK, SYNE2, TEK, THSD7B, TLN2, TMC1, TNFSF11, TRHDE, ULK4, VPS13B, YTHDC2,
ZBTB49, ZFPM2
>102
ADAM23, adenosine triphosphate, AHI1, ALDH3A2, ALDH5A1, ANK3, ASIC2, ATRX, BRAF, CDH2, CNTNAP2, CYP19A1, DAB1, DDIT4,
DISC1, EGR2, EN1, EPHA7, ERBB3, ERBB4, FAT4, FOXP2, IL1RAPL1, IL1RAPL2, IMMP2L, LHX8, LRRK2, PARD3, PPT1, RAPGEF2,
ROBO2, SLC6A3, STAM, TENM2, TENM4, ULK4, VAX1, VWC2
>103
ABCB1, AGBL4, ANO3, BRAF, CASP8, CCDC178, CDH10, CDH13, CDH2, CNBD1, CTNND2, CUBN, CXCL13, DAB1, DCC, DOCK4, EPHA7,
ERBB3, ERBB4, EYS, FAT4, HMCN1, HMGCR, ITGA8, KCTD16, MMP2, MYRIP, NFKB1, PDPN, PDS5B, PLOD2, PXDNL, ROBO2,
SEMA6D, SLC6A3, STXBP5L, SYNE2, TCHH, THSD7B, TRHDE, UTRN
>104
ACSL4, ADAMTS6, AHI1, CAPN3, CASP8, CD22, CD28, CFH, CNTNAP2, COL19A1, CPNE4, CTSB, CXCL13, CYBB, CYP19A1, DAB1,
DDAH1, DLGAP2, DYM, ERBB3, FGF14, FSTL4, GABRA2, GABRB1, GABRB3, GABRG2, GRIK2, GRM8, HCN1, HMGCR, HPGD, HTR3B,
HYAL1, HYOU1, ICOS, IL15, ITGAM, ITGAX, ITPR2, JAZF1, KCNE1, KCTD16, LARS2, MERTK, NFKB1, NRG3, PDE11A, PDE4D,
phosphate, PPARGC1A, PPP2R2B, PTPN22, PXDNL, ROBO2, RTP4, SGCZ, SLCGA3, SOD1, SQSTM1, TEK, TNFSF11, TUSC3, VCAM1
>105
ABCA10, ABCA12, ABCA13, ABCA4, ABCB1, ABCC6, ACADS, ADAMTS6, ADCY8, AGK, AKAP9, ALDH5A1, ANK3, ANKRD30A, ANKS1B,
ANO3, ATRX, BBS9, BOD1, BRAF, CADM2, CASP8, CCDC7, CCSER1, CDH10, CDH13, CDH2, CFH, CHRNA7, CLN3, CNTN5, CNTNAP2,
COL19A1, CPNE4, CTNND2, CTSB, CUBN, CXCL13, DCC, DLGAP2, DNAH11, DNAII14, DNM3, DOCK4, EGR2, EPIIX1, EPIIX2, ERBB3,
ERBB4, EVC, EYS, FAF1, FAT4, FUT8, GABRB3, GALNT2, GPC5, GRID1, GRM5, GRM8, GTDC1, GYS2, HCN1, HMCN1, HMGN5, IL15,
ITGA8, ITGAX, JAZF1, KCNN2, KCTD8, KDM6A, KIF26B, KRT6B, KRTAP9-2, KRTAP9-4, KRTAP9-8, LBR, LMO1, LRRK2, MAST4,
MB21D2, MMP2, MTUS2, NCOA3, NELL1, NUDT13, OCA2, OPHN1, OSBPL1A, OTOL1, PARD3, PDE11A, PDPN, PDZRN4, PKHD1, PLOD2,
PPARGC1A, PPP2R2B, PROS1, PTPRD, PTPRN2, PXDNL, RIIAG, ROBO2, SCG3, SGCZ, SLCO3A1, SOD1, STAG2, STXBP5L, SYK, SYNE2,
TCHH, TEK, TENM2, THOP1, THSD7B, TLN2, TNFSF11, TRHDE, TRIM36, UTRN, VAV3, VCAM1, VPS13B, WDPCP, WDR27, WWTR1,
XKR4, YTHDC2, ZPLD1
>106
ABCB1, ADCY8, AHI1, ALOX5, CCDC7, CD28, CTNNA3, CTSB, CXCL13, CYP19A1, DYM, EPHX2, EVC, FAF1, GABRA2, GABRB,
GABRB3, GABRG2, GALNT2, HMGCR, HTR3B, ICOS, IL15, LRRK2, MMP2, NELL1, NFKB1, OCA2, PDE11A, PPP2R2B, PTPN22, PTPRN2,
RPL11, SIL1, SYK, TNFSF11, TPST1, VCAM1, YTHDC2
>107
ABCA10, ABCA4, ABCB1, ABCC6, ACADS, AGXT, AHI1, ALDH5A1, ANK3, ARHGEF4, BRAF, CCDC137, CCT8, CD22, CNTNAP2, CTNNA3,
CTNND2, CYP19A1, CYP1A1, DISC1, DNAH14, EPHA7, EPHX2, ERBB3, EYS, HTR3B, HYOU1, ITGAM, LRRK2, MASP2, MERTK, MTUS2,
NCOA3, NSUN3, OCA2, OSBPL1A, PHACTR1, PPARGC1A, PTPN12, PTPRN2, ROBO2, SEMA6D, SLC24A3, STXBP5L, TCHH, TEK, TMC1,
TRIMS, VAV3, VPS13B, WDPCP, WDK27, ZEPM2
>108
ABCA12, ABCA4, ABCB1, ABCC6, ACADSB, ACOT7, ACSL4, ACSS2, adenosine triphosphate, AGK, ALDH5A1, ALOX5, CASP8, CASR, CLN3,
CYP1A1, DAB1, DLD, EPHX1, EPHX2, ERBB4, HPGD, HSD17B4, IL15, LIPG, NFKB1, OSBPL1A, PARD3, PCTP, PDPN, PEX5,
PPAKGC1A, PKKAG2, SC5D, SLCO3A1, SOD1, SUCLG2, SYK
>109
ABCA10, ABCA12, ABCA4, ABCB1, ACADS, ACSS2, ADAM23, ADAMTS6, AGK, AKAP9, ANKRD30A, ANKS1B, ANO3, ATRX, BMPR2,
BRAF, C12orf42, CAMKMT, CASP8, CCDC7, CD22, CDH10, CDH2, CFH, CNTLN, CNTNAP2, CTNND2, CUBN, CYP19A1, CYP1A1, CYP3A5,
DD1T4, DNAH11, DNAH14, DNAJC13, DNM3, DYSF, EGR2, EN1, EPHX1, EPHX2, ERBB3, ERBB4, ESRRG, EVC, EAM122C, EAT4,
FRAS1, GRID1, GRIK2, GRM8, GSTM2, HCN1, HIVEP1, HMCN1, HMGCR, HMGN5, IL1RAPL1, ITGAX, KCNH8, KCTD16, KDM6A, KHT6B,
KRTAP9-9, LINGO2, LMO1, LRRK2, MAST4, MB21D2, MCCC1, MMP2, MTUS2, NCOA3, NUDT13, OCA2, OPHN1, OSBPL1A, PDPN,
PDS5B, PHACTR1, PKHD1, PRKAG2, PTPN22, PTPRD, PTPRN2, PXDNL, ROBO2, SIL1, SLC25A12, SLCO3A1, SOD1, STAG2, TCHH, TEK,
TENM2, THSD7B, TLN2, TNFSF11, TRHDE, TRIM36, ULK4, UTRN, VAV3, VCAM1, VPS13B, WDPCP, WWTR1, ZFPM2, ZNF608, ZPLD1
>110
ABCA4, ABCC6, adenosine triphosphate, ADGRV1, AHI1, ANK3, ATRX, BARX1, BNC2, BRAF, CDH2, CFH, CLN3, CNTNAP2, CYP19A1,
DAB1, DDIT4, EGR2, EN1, EPHA7, EPHX2, ERBB4, FAT4, FOXP2, FRAS1, GABRB3, HCN1, IL1RAPL1, IMMP2L, ITGA8, LHX8, LRRK2,
MERTK, MMP2, NPHP1, PARD3, PPT1, RAPGEF2, ROBO2, SLC6A3, SOD1, STAM, TBC1D32, TENM2, ULK4, VAX1, VCAM1, VWC2,
WDPCP
>111
ADCY8, ADGRB3, ANK3, BRAF, CHRNA7, CLN3, CNTNAP2, CRHR1, CTNND2, CYBB, CYP19A1, EN1, GABRB3, GABRG2, GRID2, GRM4,
GRM5, HCN1, HMGCR, ITGA8, LHX8, NFKB1, OPHN1, PDE11A, PDE4D, PPARGC1A, PPT1, PTPRN2, SLC6A3, TUSC3
>112
ABCA4, adenosine triphosphate, ADGRV1, AGBL4, AHI1, ANK3, ASIC2, CASP8, CFH, CLN3, CRHR1, CTSB, CYBB, ESRRG, EYS, GABRB3,
GRID1, GRID2, HGSNAT, HMCN1, HSD17B4, LRRK2, MERTK, MFSD8, NFKB1, NPHP1, PPARGC1A, PPT1, PRPF8, RAB28, SOD1,
SQSTM1
>113
ABCB1, AGBL4, ANO3, BRAF, CCDC178, CDH10, CDH13, CDH2, CNBD1, CTNND2, CUBN, CXCL13, DAB1, DCC, DOCK4, EPHA7, ERBB3,
ERBB4, EYS, FAT4, HMCN1, ITGA8, KCTD16, MMP2, MYRIP, PDPN, PDS5B, PLOD2, PXDNL, ROBO2, SEMA6D, SLC6A3, STXBP5L,
SYNE2, TCHH, THSD7B, TRHDE
>114
ALDH5A1, CDH2, CLN3, CNTNAP2, CTSB, DNAI1, HSD17B4, ITPR2, LRRK2, PPARGC1A, PPT1, SOD1, SQSTM1, STAM
>115
ABCA4, ABCC6, adenosine triphosphate, ADGRV1, AHI1, ANK3, ATRX, BARX1, BMPR2, BNC2, BRAF, CDH2, CFH, CLN3, CNTNAP2,
CYP19A1, DAB1, DDIT4, DNAH11, EGR2, EN1, EPHA7, EPHX2, ERBB4, FAT4, FOXP2, FRAS1, GABRB3, HCN1, IL1RAPL1, IMMP2L,
ITGA8, LHX8, LRRK2, MERTK, MMP2, NPHP1, PARD3, PPT1, RAPGEF2, ROBO2, SLC6A3, SOD1, STAM, TBC1D32, TENM2, ULK4,
VAX1, VCAM1, VWC2, WDPCP
>116
ABCA10, ABCA13, ABCA4, ABCB1, ACADSB, ANK3, ANKRD30A, ARHGAP24, BRAF, CCDC7, CD22, CFH, CNTLN, CNTN5, CTNNA3,
CTNND2, CUBN, CWC27, CYP1A1, CYP3A5, DNAH11, DNAH14, DYSH, EPHX1, EPHX2, FAT4, FRAS1, GRIK2, HIVEP1, HMCN1, IL15,
KDM6A, LMO1, MERTK, MMP2, NCOA3, NEK11, OSBPL1A, OTOL1, PARD3, PHACTR1, PKHD1, PPP2R2B, PRPFS, PTPRD, PXDNL,
RPL11, SGCZ, SLC22A20, SLC24A3, SLC6A3, SPAG17, STAG2, STAM, SYK, SYNE2, TEK, THSD7B, TLN2, TNFSF11, ULK4, VPS13B,
YTHDC2, ZBTB49, ZFPM2
>117
ALDH5A1, ANK3, C4orf22, CHRNA7, CNBD1, GABRA2, GABRB1, GABRB3, GABRG2, GRID1, GRIK2, GRM4, GRM5, HMGCR, MCCC1,
NEK11, NKAIN2, SLC6A3, SYK, TENM4
>118
ADCY8, ALOX5, BMPR2, CASP8, CFH, CHRNA7, CTSB, CYBB, CYP1A1, EGR2, GABRA2, GABRB1, GABRB3, GABRG2, HMGCR, HYOU1,
IL15, LBR, NFKB1, NPR3, PDE4D, PROS1, PTPN20, SLC6A3, SYK, TNESF11, VCAM1
>119
AD CY8, ADGRB3, BRAF, CHRNA7, CLN3, CNTNAP2, CRHR1, CTNND2, CYBB, CYP19A1, EN1, GABRB3, GABRG2, GRID2, GRM4, GRM5,
HCN1, HMGCR, ITGA8, LHX8, NFKB1, OPHN1, PDE11A, PDE4D, PPARGC1A, PPT1, PTPRN2, SLC6A3
>120
ABCA10, ABCA13, ABCA4, ARCB1, ACADSB, AHI1, ANK3, ANKRD30A, ARHGAP24, BRAF, CCDC7, CD22, CD28, CDH10, CFH, CNTLN,
CNTN5, CNTNAP2, CTNNA3, CTNND2, CUBN, CWC27, CXCL13, CYP1A1, CYP3A5, DLEU1, DNAH11, DNAH14, DYSF, EPHX1, EPHX2,
FAT4, FRAS1, GRIK2, HIVEP1, HMCN1, IL15, KDM6A, LMO1, LRRK2, MERTK, MMP2, NCOA3, NEK11, OSBPL1A, OTOL1, PARD3,
PDE4D, PHACTR1, PKHD1, PPP2R2B, PRPF5, PTPRD, PXDNL, RPL11, SGCZ, SLC22A20, SLC24A3, SLC6A3, SPAG17, STAG2, STAM,
SYK, SYNE2, TEK, THSD7B, TLN2, TMC1, TNFSF11, TRHDE, ULK4, VPS13B, YTHDC2, ZBTB49, ZFPM2
>121
ABCA10, ABCA12, ABCA13, ABCA4, ABCB1, ABCC6, ACADS, ADAMTS6, ADCY8, AGK, AKAP9, ALDH5A1, ANK3, ANKRD30A, ANKS1B,
ANO3, ATRX, BBS9, BOD1, BRAF, CADM2, CASP8, CCDC7, CCSER1, CDH10, CDH13, CDH2, CFH, CHRNA7, CLN3, CNTN5, CNTNAP2,
COL19A1, CPNE4, CTNND2, CUBN, CXCL13, DCC, DLGAP2, DNAH11, DNAH14, DNM3, DOCK4, EGR2, EPHX1, EPHX2, ERBB3, ERBB4,
EVC, EYS, FAT4, FUT8, GABRB3, GALNT2, GPC5, GRID1, GRM5, GRM8, GTDC1, GYS2, HCN1, HMCN1, HMGN5, ITGA8, ITGAX, JAZF1,
KCNN2, KCTD8, KDM6A, KIF26B, KRT6B, KRTAP9-2, KRTAP9-4, KRTAP9-8, LBR, LRRK2, MAST4, MB21D2, MTUS2, NCOA3, NELL1,
NUDT13, OCA2, OPHN1, OSBPL1A, OTOL1, PDE11A, PDPN, PDZRN4, PKHD1, PLOD2, PPARGC1A, PPP2R2B, PROS1, PTPRD, PTPRN2,
PXDNL, RHAG, ROBO2, SCG3, SGCZ, SLCO3A1, SOD1, STAG2, STXBP5L, SYK, SYNE2, TCHH, TENM2, THOP1, THSD7B, TLN2, TRHDE,
TRIM36, UTRN, VCAM1, VPS13B, WDPCP, WDR27, WWTR1, XKR4, YTHDC2, ZPLD1
>122
adenosine triphosphate, ADGRB3, ADGRL2, ASIC2, CASR, CDII2, CIIRNA7, CYP19A1, DCC, DISC1, DNM3, DOCK4, EPIIA7, ERBB4, GRID2,
IL1RAPL1, LINGO2, LRRK2, PARD3, RAPSN, UTRN
>123
ADGRB3, ADGRL2, ASIC2, CDH2, CHRNA7, CYP19A1, DCC, DISC1, DNM3, EPHA7, ERBB4, GRID2, IL1RAPL1, LINGO2, LRRK2, PARD3,
RAPSN, UTRN
>124
ABCB1, ADAM23, adenosine triphosphate, ADH1C, ALDH5A1, ANK3, ANO3, CAPN3, CASP8, CDH2, CHRNA7, CLN3, CRYM, CTNNA3,
CTSB, CYP19A1, DAB1, DCC, DNAJC13, EGR2, EPHX2, ERBB3, ESRRG, FGF14, FOXN3, GABRA2, GABRB1, GABRB3, GABRG2, GPR6,
GRID2, GRIK2, IIMGCR, IISD17B4, ITGAM, LRRK2, MERTK, NFKB1, PCTP, PDE4D, phosphate, PLOD2, PPARGC1A, PPP2R2B, PPT1,
PTPRD, SIL1, SLC6A3, SOD1, SQSTM1, WWTR1, XK
>125
adenosine triphosphate, ADGRB3, AHI1, AKAP9, ANK3, ARHGAP24, ARHGEF4, BBS5, BBS9, BRAF, CD2AP, CDH13, CDH2, CNTNAP2,
CRIIR1, CTNND2, CYP19A1, DAB1, DCC, DDAII1, DISC1, DNM3, DOCK4, EPIIA7, EPIIX2, ERBB4, FUT8, GRID2, GRM4, GRM5, ICOS,
IL1RAPL1, ITGA8, ITGAM, LRRK2, MTUS2, NFKB1, OPHN1, PARD3, PDPN, PKHD1, PTPN20, RAPGEF2, RAPSN, RIT2, RNF19A, ROBO2,
SOD1, SYK, TBC1D32, TENM4, TNFSF11, ULK4, VAV3, WDPCP, WWTR1
>126
AGBL4, ANK3, CRIIR1, CTSB, CYBB, GRID2, IISD17B4, LRRK2, NEKB1, PPARGC1A, PPT1, SOD1, SQSTM1
>127
ABCB1, AGBL4, ANO3, BRAF, CCDC178, CDH10, CDH2, CNBD1, CTNND2, DAB1, DOCK4, EPHA7, EYS, FAT4, HMCN1, KCTD16, MYRIP,
PDS5B, PXDNL, SEMA6D, SLC6A3, STXBP5L, SYNE2, THSD7B, TRHDE
>128
adenosine triphosphate, ADGRB3, AHI1, AKAP9, ANK3, ARHGAP24, ARHGEF4, BBS5, BBS9, BRAF, CD2AP, CDH13, CDH2, CLN3,
CNTNAP2, CRHR1, CTNND2, CYP19A1, DAB1, DCC, DDAH1, DISC1, DNAJC13, DNM3, DOCK4, DYM, EPHA7, EPHX2, ERBB4, FUT8,
GRID2, GRM4, GRM5, ICOS, IL1RAPL1, ITGA8, ITGAM, KRT6B, LRRK2, MERTK, MFSD8, MTUS2, NFKB1, OPHN1, PARD3, PDPN, PEX5,
PHACTR1, PKHD1, PPARGC1A, PPT1, PTPN20, RAPGEF2, RAPSN, RIT2, RNF19A, ROBO2, SOD1, SYK, TBC1D32, TENM4, TNFSF11,
ULK4, UVRAG, VAV3, WDPCP, WWTR1
>129
ABCA12, ABCA4, ABCB1, ADGRV1, BRAF, CAPN3, CD22, CD28, CD2AP, CDH2, CFH, CHRNA7, CLN3, CNTNAP2, CTNNA3, CTSB, CYBB,
CYP19A1, DNM3, DYSF, EPHA7, ERBB3, GRID2, HSD17B4, HYAL1, IL15, IQGAP2, ITGA8, ITPR2, KRT6B, LHX8, LRRK2, MERTK,
NFKB1, NPHP1, NPR3, OPHN1, OSCAR, PARD3, PCTP, PDPN, PDS5B, PPARGC1A, PPT1, PTPN22, RAPSN, ROBO2, SC5D, SOD1, SYK,
SYNE2, TDRKH, TEK, TNFSF11, UTRN, VAV3, XK
>130
ADGRB3, ADGRL2, ASIC2, CASR, CDH2, CHRNA7, CYP19A1, DCC, DISC1, DNM3, EPHA7, ERBB4, GRID2, IL1RAPL1, LINGO2, LRRK2,
PARD3, RAPSN, TLN2, UTRN
>131
AHI1, ANK3, ASIC2, CHRNA7, CLN3, CNTNAP2, CTSB, DAB1, DCC, EN1, EPHA7, ERBB3, ERBB4, GABRB3, GRID1, GRID2, IL1RAPL1,
MERTK, OPHN1, PEX5, PPARGC1A, PPT1, PROS1, ROBO2, SLC6A3, TENM2, ULK4, VAX1
>132
ABCB1, AGBL4, ANO3, BRAF, CCDC178, CDH10, CDH2, CNBD1, CTNND2, CUBN, DAB1, DCC, DOCK4, EPHA7, EYS, FAT4, HMCN1,
KCTD16, MYRIP, PDS5B, PXDNL, SEMA6D, SLC6A3, STXBP5L, SYNE2, TCHH, THSD7B, TRHDE
>133
ABCB1, ALDH5A1, ANK3, C4orf22, CHRNA7, CNBD1, GABRA2, GABRB1, GABRB3, GABRG2, GRID1, GRIK2, GRM4, GRM5, HMGCR,
HTR3B, MCCC1, NEK11, NKAIN2, PDE11A, SLC6A3, SYK, TENM4, VPS13B
>134
ABCB1, ADCY8, AHI1, ALOX5, CASP8, CCDC7, CD28, CTNNA3, CTSB, CXCL13, CYP19A1, DYM, EPHX2, ERBB3, EVC, FAF1, GABRA2,
GABRB1, GABRB3, GABRG2, GALNT2, HMGCR, HPGD, HTR3B, HYAL1, ICOS, IL15, ITGAM, LRRK2, MERTK, MMP2, NELL1, NFKB1,
OCA2, PDE11A, PDE4D, PPP2R2B, PTPN22, PTPRN2, RPL11,
SIL1, SLC6A3, SYK, TNFSF11, TPST1, VCAM1, YTHDC2
>135
AHI1, ANK3, ASIC2, CHRNA7, CLN3, CNTNAP2, CTSB, DAB1, DCC, EN1, EPHA7, ERBB3, ERBB4, GABRB3, GRID2, IL1RAPL1, MERTK,
OPHN1, PEX5, PPARGC1A, PPT1, PROS1, ROBO2, SLC6A3, TENM2, ULK4, VAX1
>136
ADGRB3, ADGRL2, ASIC2, CASR, CDH2, CHRNA7, CYP19A1, DCC, DISC1, DNM3, DOCK4, EPHA7, ERBB4, GRID2, IL1RAPL1, LINGO2,
LRRK2, PARD3, RAPSN, UTRN
>137
ABCA10, ABCA12, ABCA4, ABCB1, ACADS, ACSS2, AGK, ANKRD30A, ANO3, ATRX, BRAF, CASP8, CCDC7, CDH10, CDH2, CFH,
CNTNAP2, CTNND2, CUBN, CYP19A1, DNAH11, DNAH14, DNM3, EGR2, EPHX1, ERBB3, EVC, FAM122C, FAT4, GRID1, HIVEP1,
HMCN1, HMGN5, ITGAX, KDM6A, KRT6B, LRRK2, MCCC1, MMP2, MTUS2, NCOA3, OCA2, OPHN1, OSBPL1A, PDE4D, PDS5B, PKHD1,
PTPRN2, PXDNL, ROBO2, SLC25A12, SLCO3A1, STAG2, TENM2, THSD7B, TLN2, TRHDE, TRIM36, TUSC3, ULK4, UTRN, VAV3, VCAM1,
WDPCP, ZFPM2, ZPLD1
>138
ABCA10, ABCA12, ABCA13, ABCA4, ABCB1, ACADSB, AHI1, ALOX5, ANK3, ANKRD30A, ARHGAP24, BMPR2, BRAF, CASP8, CCDC7,
CD22, CD28, CDH10, CFH, CNTLN, CNTN5, CNTNAP2, CTNNA3, CTNND2, CUBN, CWC27, CXCL13, CYP1A1, CYP3A5, DCC, DLEU1,
DNAH11, DNAH14, DNM3, DYSF, EGR2, EPHX1, EPHX2, FAT4, FRAS1, GRIK2, HIVEP1, HMCN1, HMGCR, IL15, KDM6A, LARS2, LMO1,
LRRK2, MERTK, MMP2, NCOA3, NEK11, NFKB1, OSBPL1A, OTOL1, PARD3, PDE4D, PHACTR1, PKHD1, PLOD2, PPP2R2B, PRPF8,
PTPRD, PXDNL, RPL11, SGCZ, SLC22A20, SLC24A3, SLC6A3, SPAG17, STAG2, STAM, SYK, SYNE2, TEK, THOP1, THSD7B, TLN2, TMC1,
TNFSF11, TRHDE, ULK4, VPS13B, YTHDC2, ZBTB49, ZFPM2
>139
ABCA10, ABCA12, ABCA4, ACADS, AGK, ANKRD30A, ANO3, ATRX, BRAE, CASP8, CCDC7, CDH10, CDH2, CFH, CNTNAP2, CTNND2,
CUBN, CYP19A1, DNAH11, DNAH14, DNM3, EGR2, EPHX1, EVC, FAM122C, FAT4, GRID1, HIVEP1, HMCN1, HMGN5, ITGAX, KDM6A,
KRT6B, LRRK2, MCCC1, MMP2, MTUS2, NCOA3, OCA2, OPHN1, OSBPL1A, PKHD1, PTPRN2, PXDNL, ROBO2, SLC25A12, SLCO3A1,
STAG2, TENM2, THSD7B, TLN2, TRHDE, TRIM36, ULK4, UTRN, VAV3, WDPCP, ZFPM2, ZPLD1
>140
ABCA10, ABCA12, ABCA13, ABCA4, ABCB1, ACADSB, adenosine triphosphate, AHI1, ALOX5, ANK3, ANKRD30A, ARHGAP24, ATRX,
BMPR2, BRAF, CASP8, CCDC7, CD22, CD28, CDH10, CFH, CNTLN, CNTN5, CNTNAP2, CTNNA3, CTNND2, CUBN, CWC27, CXCL13,
CYP1A1, CYP3A5, DCC, DLEU1, DNAH11, DNAH14, DNM3, DYSF, EGR2, EPHX1, EPHX2, FAT4, FRAS1, GRIK2, HIVEP1, HMCN1,
HMGCR, IL15, KDM6A, LARS2, LMO1, LRRK2, MERTK, MMP2, NCOA3, NEK11, NFKB1, NRG3, OSBPL1A, OTOL1, PARD3, PDE4D,
PHACTR1, PKHD1, PLOD2, PPP2R2B, PRPF8, PTPRD, PXDNL, RPL11, SGCZ, SLC22A20, SLC24A3, SLC6A3, SPAG17, STAG2, STAM,
SYK, SYNE2, TEK, THOP1, THSD7B, TLN2, TMC1, TNFSF11, TNNI3K, TRHDE, ULK4, VPS13B, YTHDC2, ZBTB49, ZFPM2
>141
adenosine triphosphate, ADGRB3, AHI1, AKAP9, ANK3, ARHGAP24, ARHGEF4, BBS5, BBS9, BRAF, CD2AP, CDH13, CDH2, CLN3,
CNTNAP2, CRHR1, CTNND2, CYP19A1, DAB1, DCC, DDAH1, DISCI, DNM3, DOCK4, EPHA7, EPHX2, ERBB4, FUT8, GRID2, GRM4,
GRM5, ICOS, IL1RAPL1, ITGA8, ITGAM, KRT6B, LRRK2, MERTK, MTUS2, NFKB1, OPHN1, PARD3, PDPN, PHACTR1, PKHD1, PTPN20,
RAPGEF2, RAPSN, RIT2, RNF19A, ROBO2, SOD1, SYK, TBC1D32, TENM4, TNFSF11, ULK4, UVRAG, VAV3, WDPCP, WWTR1
>142
ABCA10, ABCA12, ABCA4, ABCB1, ACADS, ACSS2, AGK, ANKRD30A, ANO3, ATRX, BRAF, CASP8, CCDC7, CDH10, CDH2, CFH,
CNTNAP2, CTNND2, CUBN, CYP19A1, DNAH11, DNAH14, DNM3, EGR2, EPHX1, ERBB3, EVC, FAM122C, FAT4, GRID1, HIVEP1,
HMCN1, HMGN5, ITGAX, KDM6A, KRT6B, LRRK2, MCCC1, MMP2, MTUS2, NCOA3, OCA2, OPHN1, OSBPL1A, PDE4D, PDPN, PDS5B,
PKHD1, PTPRN2, PXDNL, ROBO2, SLC25A12, SLCO3A1, STAG2, TENM2, THSD7B, TLN2, TRHDE, TRIM36, TUSC3, ULK4, UTRN, VAV3,
VCAM1, WDPCP, ZFPM2, ZPLD1
>143
adenosine triphosphate, AHI1, ANK3, ATRX, BRAF, CDH2, CNTNAP2, CYP19A1, DAB1, DDIT4, EGR2, EN1, EPHA7, ERBB4, FAT4, FOXP2,
IL1RAPL1, IMMP2L, LHX8, LRRK2, PARD3, PPT1, RAPGEF2, ROBO2, SLC6A3, STAM, ULK4, VAX1, VWC2
>144
ABCA12, ADAMTSG, BARX1, BMPR2, BRAF, CAPN3, CASP8, CRIIR1, CYP19A1, DMRTA1, DNAII11, DNAI1, EGR2, EN1, ERBB3, ERBB4,
ESRRG, FAT4, FRAS1, FUT8, GRID2, HYAL1, IL15, IMMP2L, ITGA8, KDM6A, LALBA, LHX8, LRRK2, MMP2, NCOA3, NFKB1, NRG3,
PARD3, PDPN, PDS5B, PKHD1, PPARGC1A, PTPN12, PTPRN2, RAPGEF2, ROBO2, SLC6A3, SOD1, SYK, TBC1D32, TEK, TNFSF11,
TSC22D1, UTRN, VCAM1, WDPCP, WWTR1, ZFPM2
>145
ABCB1, AGBL4, ANO3, BRAF, CCDC178, CDH10, CDH2, CNBD1, CTNND2, CUBN, DAB1, DCC, DOCK4, EPHA7, EYS, FAT4, HMCN1,
HMGCR, KCTD16, MYRIP, PDS5B, PXDNL, SEMA6D, SLC6A3, STXBP5L, SYNE2, TCHH, THSD7B, TRHDE
>146
ABCA12, ABCA4, ABCB1, ABCC0, ACADS, ACSL4, adenosine triphosphate, ADGRL2, AGK, AGXT, AIII1, AKAP9, ALOX5, ANK3, ASIC2,
BRAF, CASR, CD22, CD28, CES3, CFH, CHRNA7, CLN3, CRHR1, CRYM, CUBN, EGR2, EPHX1, ERBB3, ERBB4, FGF14, FRAS1, GABRA2,
GABRB1, GABRB3, GABRG2, GIF, GLRA2, GOSR1,
GRID2, GRIK2, GRM4, GRM8, HTR3B, ICOS, ITPR2, LIPG, NPR3, OSBPL1A, P3H1, PCTP, PEX5, phosphate, PLCL1, PPARGC1A, PPT1,
PRKAG2, PTPRN2, RIIAG, SLC24A3, SLC25A12, SLC6A3, SLCO3A1, SOD1, SQSTM1, STXBP5L, SYK, TEK, TUSC3, XK
>147
AGBL4, ANK3, CRHR1, CTSB, CYBB, GRID2, HSD17B4, LRRK2, NFKB1, PPARGC1A, PPT1, SOD1
>148
ABCB1, ADCY8, AIII1, ALOX5, CASP8, CCDC7, CD28, CTNNA3, CTSB, CXCL13, CYP19A1, DYM, EPIIX2, EVC, FAF1, GABRA2, GABRB1,
GABRB3, GABRG2, GALNT2, HMGCR, HTR3B, HYAL1, ICOS, IL15, ITGAM, LRRK2, MERTK, MMP2, NELL1, NFKB1, OCA2, PDE11A,
PDE4D, PPP2R2B, PTPN22, PTPRN2, RPL11, SIL1, SLC6A3, SYK, TNFSF11, TPST1, VCAM1, YTHDC2
>149
CDH2, CTSB, ITPR2, SOD1
>150
ABCB1, ALDH5A1, ALOX5, CHRNA7, CYP19A1, EPHX2, GABRA2, GABRB1, GABRB3, GABRG2, GRM4, GRM5, HMGCR, HTR3B, HYAL1,
KCNE1, PDE11A, PDE4D, SLC6A3, TEK, TNFSF11
>151
ABCA10, ABCA12, ABCA4, ABCB1, ACADS, AGK, ANKRD30A, ANO3, ATRX, BRAF, CASP8, CCDC7, CDH10, CDH2, CFH, CNTNAP2,
CTNND2, CUBN, CYP19A1, DNAH11, DNAH14, DNM3, EGR2, EPHX1, ERBB3, EVC, FAM122C, FAT4, GRID1, HIVEP1, HMCN1, HMGN5,
ITGAX, KDM6A, KRT6B, LRRK2, MCCC1, MMP2, MTUS2, NCOA3, OCA2, OPHN1, OSBPLIA, PDS5B, PKHD1, PTPRN2, PXDNL, ROBO2,
SLC25A12, SLCO3A1, STAG2, TENM2, THSD7B, TLN2, TRHDE, TRIM36, ULK4, UTRN, VAV3, VCAM1, WDPCP, ZFPM2, ZPLD1
>152
ABCA12, ABCA13, ABCA4, ABCB1, ACADSB, AHI1, ANK3, ANKRD30A, ARHGAP24, ATRX, BMPR2, BRAF, CCDC7, CFH, CNTLN, CNTN5,
CTNNA3, CTNND2, CUBN, CWC27, CYP1A1, DNAH11, DNAH14, DYSF, EPHX1, EPHX2, FAT4, FRAS1, GRIK2, HIVEP1, IL15, KDM6A,
LARS2, MERTK, MMP2, NEK11, NFKB1, OSBPL1A, OTOL1, PDE4D, PHACTR1, PKHD1, PLOD2, PPP2R2B, PRPF8, PXDNL, RPL11, SGCZ,
SLC22A20, SLC24A3, SPAG17, STAG2, STAM, SYNE2, TEK, THSD7B, TLN2, TNFSF11, ULK4, VPS13B, YTHDC2, ZBTB49, ZFPM2
>153
ABCB1, adenosine triphosphate, ALDH5A1, CHRNA7, CRHR1, CYP19A1, CYP1A1, GABRA2, GABRB1, GABRB3, GABRG2, GRM5, HMGCR,
HTR3B, ITPR2, PDE11A, phosphate, SLC6A3, VPS13B
>154
AGXT, CASR, CD28, CYP1A1, GABRA2, GABRB1, GABRB3, GABRG2, HMGCR, ITGAM, KCNE1, MMP2, NPR3, PDE11A, PDE4D,
phosphate, PVT1, SLC6A3
>155
ALOX5, BRAF, CHRNA7, GABRA2, GABRB1, GABRB3, GABRG2, HMGCR, PDE4D, PROS1, SYK, TEK, ULK4
>156
ABCA10, ABCA13, ABCA4, ABCB1, ACADSB, AHI1, ALOX5, ANK3, ANKRD30A, ARHGAP24, BRAF, CASP8, CCDC7, CD22, CD28, CDH10,
CFH, CNTLN, CNTN5, CNTNAP2, CTNNA3, CTNND2, CUBN, CWC27, CXCL13, CYP1A1, CYP3A5, DCC, DLEU1, DNAH11, DNAH14,
DNM3, DYSF, EGR2, EPHX1, EPHX2, FAT4, FRAS1, GRIK2, HIVEP1, HMCN1, HMGCR, IL15, KDM6A, LMO1, LRRK2, MERTK, MMP2,
NCOA3, NEK11, NFKB1, OSBPL1A, OTOL1, PARD3, PDE4D, PHACTR1, PKHD1, PPP2R2B, PRPF8, PTPRD, PXDNL, RPL11, SGCZ,
SLC22A20, SLC24A3, SLC6A3, SPAG17, STAG2, STAM, SYK, SYNE2, TEK, THOP1, THSD7B, TLN2, TMC1, TNFSF11, TRHDE, ULK4,
VPS13B, YTHDC2, ZBTB49, ZFPM2
>157
ABCB1, ACOT7, ALDH5A1, ANK3, CHRNA7, CNTNAP2, CRYM, CTSB, CYP3A5, DAB1, DISC1, ERBB3, ERBB4, GABRA2, GABRB1,
GABRB3, GABRG2, GIF, GRID1, GRM4, GRM5, HMGCR, NELL1, PRKAG2, RIT2, SLC6A3, THOP1
>158
ABCB1, ADGRB3, BRAF, CADM2, CDH2, CUBN, EPHA7, ERBB3, FANCI, IQGAP2, KDM6A, LMO1, MTUS2, NFKB1, PKHD1, PRPF8,
PTPN12, PTPRD, ROBO2, SYK, TEK, VCAM1
>159
adenosine triphosphate, ANK3, CNTN5, CNTNAP2, CYBB, DCC, DNM3, EGR2, EN1, EPHA7, ERBB3, ERBB4, GABRG2, GRID1, IL1RAPL1,
LHX8, OPHN1, PTPRN2, RAPSN, SLC6A3, SOD1, SQSTM1, STAM, UTRN, VAX1
>160
ALDH3A2, ALG8, ASIC2, BMPR2, CASP8, CDH13, CFH, CHRNA7, CNTNAP2, CRHR1, CXCL13, CYBB, CYP19A1, CYP3A5, EPHX1, FAT4,
GABRB3, GRM5, HMGCR, KCNE1, NPR3, PDE11A, PDE4D, phosphate, PRELID2, PRKAG2, PTPRD, SLC6A3, SMOC2, SOD1, ULK4, VAV3,
VCAM1, WDR27
>161
AHI1, ALDH3A2, ATRX, BARX1, BBS5, BBS9, BRAF, DYM, ERBB3, EVC, FAT4, GABRB3, HSD17B4, LARS2, LBR, LHX8, LMBR1, NPHP1,
PDE4D, PDS5B, PEX5, RAPSN, SC5D, SIL1, SLC6A3, VAX1, VPS13B, WDPCP, XYLT1
>162
ABCA12, ABCB1, ACSL4, ALOX5, BARX1, BMPR2, CASP8, CD28, CD2AP, CDH2, CFH, CLN3, CRHR1, CTNNA3, CYBB, CYP19A1,
CYP1A1, DNAH11, ERBB3, ESRRG, FAT4, FUT8, HYAL1, ICOS, IL15, IQGAP2, ITGA8, KRT6B, LRRK2, MERTK, MMP2, NFKB1, PARD3,
PCTP, PDPN, PDS5B, PEX5, PKHD1, PPARGC1A, PPT1, PTPN22, ROBO2, SC5D, SOD1, SYK, TEK, TNFSF11, VAV3, VCAM1, WDPCP,
ZFPM2
>163
CASR, CD2AP, CDH10, CDH13, CDH2, CTNNA3, FAT4, GRID2, ICOS, IL1RAPL1, ITGAM, PDPN, SYK, TEK, TENM2, VCAM1
>164
ABCB1, ALDH5A1, ALOX5, ASIC2, CDH13, CNTN5, CTNNA3, CYP19A1, DOCK4, EPHX2, GABRA2, GABRB1, GABRB3, GABRG2, HMGCR,
ITGA8, ITGAM, ITGAX, LIPG, MMP2, NFKB1, NPR3, PDE11A, PDE4D, SLC6A3, SOD1, VAV3, VCAM1
>165
ABCA12, ABCA13, ABCA4, ABCB1, ACADSB, AHI1, ANK3, ANKRD30A, ARHGAP24, BMPR2, BRAF, CCDC7, CFH, CNTLN, CNTN5,
CTNNA3, CTNND2, CUBN, CWC27, CYP1A1, DNAH11, DNAH14, DYSF, EPHX1, EPHX2, FAT4, FRAS1, GRIK2, HIVEP1, IL15, KDM6A,
LARS2, MERTK, MMP2, NEK11, NFKB1, OSBPL1A, OTOL1, PDE4D, PHACTR1, PKHD1, PLOD2, PPP2R2B, PRPF8, PXDNL, RPL11, SGCZ,
SLC22A20, SLC24A3, SPAG17, STAG2, STAM, SYNE2, TEK, THSD7B, TLN2, TNFSF11, ULK4, VPS13B, YTHDC2, ZBTB49, ZFPM2
>166
ABCB1, ATRX, BRAF, CLN3, CNTN5, CNTNAP2, ERBB3, ERBB4, FUT8, GABRB3, GPC5, GRM5, GRM8, HCN1, ITGAX, KDM6A, KRT6B,
LRRK2, MB21D2, RHAG, STAG2
>167
ABCB1, AKAP9 ALDH5A1, ASIC2, CDH13, CHRNA7, CNTN5, CTNNA3, DOCK4, GABRA2, GABRB1, GABRB3, GABRG2, HMGCR, ITGA8,
ITGAM, KCNE1, NPR3, PDE11A, PDE4D, SLC6A3, SOD1
>168
ABCA12, BMPR2, CASP8, CD28, CD2AP, CDH2, CRHR1, CTNNA3, CYBB, CYP1A1, DNAH11, ERBB3, FAT4, FUT8, HYAL1, IL15, ITGA8,
LRRK2, MMP2, NFKB1, PARD3, PDPN, PDS5B, PPARGC1A, PTPN22, SYK, TEK, VAV3, VCAM1, ZFPM2
>169
ABCB1, BRAF, CADM2, CDH2, CUBN, EPHA7, ERBB3, FANCI, KDM6A, LMO1, MTUS2, NFKB1, PKHD1, PRPF8, PTPN12, PTPRD,
ROBO2, SYK, TEK
>170
AGXT, CASR, CD28, CYP1A1, GABRA2, GABRB1, GABRB3, GABRG2, HMGCR, ITGAM, MMP2, NPR3, PDE11A, PDE4D, phosphate, PVT1,
SLC6A3
>171
BRAF, FAT4, HMGCR, PARD3, PKHD1, ROBO2, WDPCP, WWTR1
>172
ABCA12, ABCB1, ACSL4, ALOX5, BARX1, BMPR2, CASP8, CD28, CD2AP, CDH2, CFH, CLN3, CRHR1, CTNNA3, CYBB, CYP19A1,
CYP1A1, DNAII11, ERBB3, ERBB4, ESRRG, FAT4, FUT8, IIYAL1, ICOS, IL15, IQGAP2, ITGA8, KRT6B, LRRK2, MERTK, MMP2, NFKB1,
PARD3, PCTP, PDPN, PDS5B, PEX5, PKHD1, PPARGC1A, PPT1, PTPN22, ROBO2, SC5D, SOD1, SYK, TEK, TNFSF11, VAV3, VCAM1,
WDPCP, ZFPM2
>173
ABCA10, ABCA12, ABCA4, ACADS, AGK, ANKRD30A, ANO3, ATRX, BRAF, CCDC7, CDII10, CDII2, CFII, CNTNAP2, CTNND2, CUBN,
DNAH11, DNAH14, DNM3, EGR2, EPHX1, EVC, FAT4, GRID1, HIVEP1, HMCN1, HMGN5, ITGAX, KDM6A, KRT6B, LRRK2, MCCC1,
MTUS2, NCOA3, OPHN1, OSBPL1A, PKHD1, PTPRN2, PXDNL, ROBO2, SLC25A12, SLCO3A1, STAG2, TENM2, THSD7B, TLN2, TRHDE,
TRIM36, UTRN, VAV3, WDPCP, ZFPM2, ZPLD1
>174
CAPN3, DYSF, EPHA7, GRID2, LRRK2, OPHN1, PARD3, PCTP, PDS5B, RAPSN, ROBO2, SOD1, SYNE2, UTRN, XK
>175
ABCA4, ACSS2, ADAM23, ADAMTS6, AGK, AKAP9, ANKRD30A, ANKS1B, ATRX, BMPR2, BRAF, C12orf42, CAMKMT, CCDC7, CD22,
CDII10, CDII2, CFII, CNTLN, CYP19A1, DNAJC13, DYSF, EN1, EPIIX1, ERBB3, ERBB4, ESRRG, FRAS1, GRIK2, GRM8, IIMGN5,
IL1RAPL1, ITGAX, KCNH8, KCTD16, KDM6A, KRTAP9-9, LINGO2, LRRK2, MAST4, MB21D2, MMP2, NCOA3, NUDT13, PDS5B, PHACTR1,
PRKAG2, PTPN22, PTPRD, ROBO2, SOD1, STAG2, TCHH, VPS13B, WWTR1, ZNF608
>176
GABRA2, GABRB1, GABRB3, GABRG2, HMGCR
>177
ABCB1, ADCY8, AHI1, ALOX5, CASP8, CCDC7, CD22, CD28, CTNNA3, CTSB, CXCL13, CYP19A1, DYM, EGR2, EPHX2, EVC, FAF1,
GABRA2, GABRB1, GABRB3, GABRG2, GALNT2, HMGCR, HTR3B, HYAL1, ICOS, IL15, ITGAM, LBR, LRRK2, MERTK, MMP2, NELL1,
NFKB1, OCA2, PDE11A, PDE4D, PPP2R2B, PTPN22, PTPRN2, RPL11, SIL1, SLC6A3, SYK, TNFSF11, TPST1, VCAM1, YTHDC2
>178
ABCB1, BRAF, CADM2, CDH2, CFH, CUBN, EPHA7, ERBB3, FANCI, KDM6A, LMO1, MTUS2, NFKB1, PKHD1, PRPF8, PTPN12, PTPRD,
HOBO2, SYK, TEK
>179
GABRA2, GABRB1, GABRB3, GABRG2, HMGCR, MMP2, VCAM1
>180
ABCA12, ACADS, ANKRD30A, BMPR2, BRAF, CASP8, CCDC105, CDH2, CFH, CPNE8, CYP19A1, CYP3A5, DCC, DDIT4, DISC1, DNAH11,
ERBB3, ESRRG, FAM184A, FOXP2, FRAS1, FUT8, GABRB1, GABRB3, HIVEP1, HMCN1, HSD17B4, ITGAM, KCTD8, KLHL3, MMP2,
NCOA3, NFKB1, PDE11A, PDS5B, PKHD1, PTPN12, PTPRD, PTPRN2, RPL11, SH3RF3, TCHH, TEK, TENM2, THSD7B, TNFSF11, TRHDE,
ULK4, UTRN, VAV3, VCAM1
>181
ERBB3, GABRA2, GABRB1, GABRB3, GABRG2, HMGCR, MMP2, PDS5B, VCAM1, ZFPM2
>182
ABCA10, BRAF, CD22, CD28, CDH10, CNTNAP2, CTNND2, CXCL13, CYP1A1, CYP3A5, DLEU1, FAT4, HIVEP1, HMCN1, IL15, KDM6A,
LMO1, LRRK2, NCOA3, PARD3, PDE4D, PTPRD, SLC6A3, SYK, SYNE2, TEK, TMC1, TNFSF11, TRHDE
>183
adenosine triphosphate, ANK3, ANKS1B, ASIC2, CASR, CDH2, CHRNA7, CLN3, CNTNAP2, CRHR1, FGF14, GABRG2, GRID2, GRIK2,
GRM4, GRM5, GRM8, HCN1, HTR3B, KCNE1, NFKB1, OPHN1, RAPSN, RIT2
>184
ABCA10, ABCA13, ABCA4, ABCB1, ACADSB, AHI1, ALOX5, ANK3, ANKRD30A, ARHGAP24, BRAF, CASP8, CCDC7, CD22, CD28, CDH10,
CFH, CNTLN, CNTN5, CNTNAP2, CTNNA3, CTNND2, CUBN, CWC27, CXCL13, CYP1A1, CYP3A5, DCC, DLEU1, DNAH11, DNAH14,
DNM3, DYSF, EGR2, EPHX1, EPHX2, FAT4, FRAS1, GRIK2, HIVEP1, HMCN1, HMGCR, IL15, KDM6A, LMO1, LRRK2, MERTK, MMP2,
NCOA3, NEK11, NFKB1, NRG3, OSBPL1A, OTOL1, PARD3, PDE4D, PHACTR1, PKHD1, PPP2R2B, PRPF8, PTPRD, PXDNL, RPL11, SGCZ,
SLC22A20, SLC24A3, SLC6A3, SPAG17, STAG2, STAM, SYK, SYNE2, TEK, THOP1, THSD7B, TLN2, TMC1, TNFSF11, TNNI3K, TRHDE,
ULK4, VPS13B, YTHDC2, ZBTB49, ZFPM2
>185
ABCA4, ABCB1, adenosine triphosphate, ALOX5, ANK3, ATRX, BMPR2, BRAF, CASP8, CD22, CD28, CDH13, CFH, CHRNA7, CLN3, CNTN5,
CNTNAP2, CTSB, CXCL13, CYBB, CYP19A1, CYP1A1, DCC, DNM3, EGR2, EN1, EPHA7, ERBB3, ERBB4, ESRRG, GABRG2, GRID1,
ICOS, IL15, IL1RAPL1, IQGAP2, ITGAM, LHX8, LIPG, LRRK2, MERTK, MMP2, NFKB1, NPHP1, NPR3, OPHN1, OSCAR, PDS5B, PLCL1,
PPARGC1A, PROS1, PTPN12, PTPN22, PTPRN2, RAPGEF2, RAPSN, SLC6A3, SOD1, SPRED2, SQSTM1, STAM, SYK, TEK, TNFSF11,
UTRN, UVRAG, VAV3, VAX1, VCAM1
>186
CDH2, CNTNAP2, CTSB, DNAI1, HSD17B4, ITPR2, PPT1, SOD1, SQSTM1
>187
ABCA12, ABCA4, ABCB1, adenosine triphosphate, ADGRV1, AHI1, ARHGAP24, ARHGEF4, BRAF, CAPN3, CASP8, CD22, CD28, CD2AP,
CDH13, CDH2, CFH, CHRNA7, CLN3, CNTNAP2, CTNNA3, CTNND2, CTSB, CYBB, CYP19A1, DCC, DNM3, DYSF, EPHA7, ERBB3,
ERBB4, GRID1, GRID2, GRM5, HSD17B4, HYAL1, IL15, IQGAP2, ITGA8, ITGAM, ITPR2, KRT6B, LHX8, LRRK2, MERTK, NCOA3, NFKB1,
NPHP1, NPR3, OPHN1, OSCAR, PARD3, PCTP, PDPN, PDS5B, PPARGC1A, PPT1, PTPN12, PTPN22, RAPGEF2, RAPSN, ROBO2, SC5D,
SOD1, SYK, SYNE2, TDRKH, TEK, TNFSF11, UTRN, VAV3, VCAM1, WWTR1, XK
>188
ABCA12, ACADS, ANKRD30A, BMPR2, BRAF, CASP8, CCDC105, CDH2, CFH, CPNE8, CYP19A1, CYP3A5, DCC, DDIT4, DISCI, DNAH11,
ERBB3, ESRRG, FAM184A, FOXP2, FRAS1, FUT8, GABRB1, GABRB3, HIVEP1, HMCN1, HSD17B4, ITGAM, KCTD8, KLHL3, MMP2,
NCOA3, NFKB1, PDE11A, PDPN, PDS5B, PKHD1, PTPN12, PTPRD, PTPRN2, RPL11, SH3RF3, TCHH, TEK, TENM2, THSD7B, TNFSF11,
TRHDE, ULK4, UTRN, VAV3, VCAM1
>189
ABCA12, ACADS, ANKRD30A, BMPR2, BRAF, CASP8, CCDC105, CDH2, CFH, CPNE8, CYP3A5, DCC, DDIT4, DISC1, DNAH11, ERBB3,
ESRRG, FAM184A, FOXP2, FRAS1, FUT8, GABRB1, GABRB3, HIVEP1, HMCN1, HSD17B4, ITGAM, KCTD8, KLHL3, MMP2, NCOA3,
NFKB1, PDE11A, PDS5B, PKHD1, PTPN12, PTPRD, PTPRN2, RPL11, SH3RF3, TCHH, TEK, TENM2, THSD7B, TNFSF11, TRHDE, ULK4,
UTRN, VAV3, VCAM1
>190
ADGRB3, ANK3, ARHGAP24, BRAF, CDH2, CNTNAP2, CRHR1, CTNND2, CYP19A1, DAB1, DCC, DDAH1, EPHA7, ERBB4, FUT8, GRID2,
GRM4, GRM5, IL1RAPL1, LRRK2, NFKB1, OPHN1, PARD3, PDPN, PTPN20, RAPGEF2, RAPSN, RIT2, ROBO2, SOD1, TENM4
>191
GABRA2, GABRB1, GABRB3, GABRG2, HMGCR
>192
GABRA2, GABRB1, GABRB3, GABRG2, HMGCR
>193
ABCA10, ABCA12, ABCA4, ACADS, AGK, ANKRD30A, ANO3, BRAF, CCDC7, CDH10, CDH2, CFH, CNTNAP2, CTNND2, CUBN, DNAH11,
DNAH14, DNM3, EGR2, EPHX1, EVC, FAT4, GRID1, HMCN1, HMGN5, ITGAX, MTUS2, NCOA3, OPHN1, OSBPL1A, PKHD1, PTPRN2,
PXDNL, ROBO2, SLCO3A1, STAG2, TENM2, TLN2, TRHDE, TRIM36, UTRN, WDPCP
>194
ABCA13, ADAM23, ADH1C, ANKRD30A, ANKS1B, ATRX, BRAF, C12orf42, CDH10, CDH12, CPXCR1, CTNND2, CUBN, DCC, DYSF,
ERBB3, ERBB4, FRAS1, FUT8, GMPR, GRID2, HSD17B4, KCNH8, KCTD16, KDM6A, KRT6B, LRRK2, MASP2, MB21D2, NCOA3,
PHACTR1, ROBO2, STAG2, SYNE2, TCHH, TRHDE, ULK4
>195
ABCA12, ACADS, ANKRD30A, BMPR2, BRAF, CASP8, CCDC105, CDH2, CFH, CPNE8, CYP3A5, DCC, DDIT4, DISC1, DNAH11, ERBB3,
ESRRG, FAM184A, FOXP2, FRAS1, FUT8, GABRB1, GABRB3, HIVEP1, HMCN1, HSD17B4, KCTD8, KLHL3, MMP2, NCOA3, NFKB1,
PDE11A, PDS5B, PKHD1, PTPN12, PTPRD, PTPRN2, RPL11, SH3RF3, TCHH, TEK, TENM2, THSD7B, TNFSF11, TRHDE ULK4, UTRN,
VAV3, VCAM1
>196
ALDII5A1, BRAF, CDII2, CLN3, CNTNAP2, CTSB, DNAI1, ERBB3, IISD17B4, IL15, ITPR2, LRRK2, NFKB1, PDE4D, PPARGC1A, PPT1,
SOD1, SQSTM1, STAM, SYK
>197
ABCB1, ALDH5A1, ALOX5, ASIC2, CDH13, CNTN5, CTNNA3, CYP19A1, DOCK4, EPHX2, GABRA2, GABRB1, GABRB3, GABRG2, HMGCR,
ITGA8, ITGAM, ITGAX, LIPG, MMP2, NFKB1, NPR3, PDE11A, PDE4D, SOD1, VCAM1
>198
CASR, CYP1A1, GABRA2, GABRB1, GABRB3, GABRG2, HMGCR, MMP2, PDE11A, PDE4D, phosphate, PVT1, SLC6A3
>199
ABCB1, ADGRV1, ALDII5A1, ANK3, CASR, CIIRNA7, CLN3, CNTNAP2, CRYM, CTSB, EGR2, EPIIX1, GABRA2, GABRB1, GABRB3,
GABRG2, GRIK2, GRM5, HCN1, HTR3B, HYOU1, PLCL1, PPT1, PTPRN2, SLC6A3, STAM
>200
ABCB1, BRAF, CADM2, CDH2, CUBN, EPHA7, ERBB3, FANCI, KDM6A, LMO1, MTUS2, NFKB1, PKHD1, PRPF8, PTPN12, PTPRD,
ROBO2, SYK
>201
CHRNA7, GABRA2, GABRB1, GABRB3, GABRG2, HMGCR, SLC6A3
>202
ABCA4, ABCB1, ACSS2, ADAM23, ADAMTS6, AGK, AKAP9, ANKRD30A, ANKS1B, ATRX, BMPR2, BRAF, C12orf42, CAMKMT, CASP8,
CCDC7, CD22, CDH10, CDH2, CFH, CNTLN, CYP19A1, CYP1A1, CYP3A5, DDIT4, DNAJC13, DYSF, EN1, EPHX1, EPHX2, ERBB3, ERBB4,
ESRRG, FRAS1, GRIK2, GRM8, GSTM2, HCN1, HMGCR, HMGN5, IL1RAPL1, ITGAX, KCNH8, KCTD16, KDM6A, KRT6B, KRTAP9-9,
LINGO2, LMO1, LRRK2, MAST4, MB21D2, MMP2, NCOA3, NUDT13, PDPN, PDS5B, PHACTR1, PRKAG2, PTPN22, PTPRD, ROBO2, SIL1,
SOD1, STAG2, TCHH, TEK, TNFSF11, UTRN, VPS13B, WWTR1, ZNF608
>203
ABCB1, ALDH5A1, ANK3, CNTNAP2, GABRA2, GABRB1, GABRB3, GABRG2, SLC6A3
>204
ABCA10, ABCA12, ABCA4, ACADS, AGK, ANKRD30A, ANO3, CCDC7, CDH10, CDH2, CFH, CNTNAP2, CTNND2, CUBN, DNAH11,
DNAH14, DNM3, EGR2, EPHX1, EVC, FAT4, GRID1, HMCN1, HMGN5, ITGAX, MTUS2, NCOA3, OPHN1, OSBPL1A, PKHD1, PTPRN2,
PXDNL, ROBO2, SLCO3A1, STAG2, TENM2, TLN2, TRHDE, TRIM36, UTRN, WDPCP
>205
ABCB1, ALDH5A1, ASIC2, CDH13, CNTN5, CTNNA3, DOCK4, GABRA2, GABRB1, GABRB3, GABRG2, HMGCR, ITGA8, ITGAM, NPR3,
PDE11A, PDE4D, SOD1
>206
ABCA12, ACADS, ANKKD30A, BMPR2, BRAF, CASP8, CCDC105, CDH2, CFH, CPNE8, CYP3A5, DCC, DDIT4, DISC1, DNAH11, ERBB3,
ESRRG, FAM184A, FOXP2, FRAS1, FUT8, GABRB1, GABRB3, HIVEP1, HMCN1, HSD17B4, KCTD8, KLHL3, MMP2, NCOA3, NFKB1,
PDE11A, PDPN, PDS5B, PKHD1, PTPN12, PTPRD, PTPRN2, RPL11, SH3RF3, TCHH, TEK, TENM2, THSD7B, TNFSF11, TRHDE, ULK4,
UTRN, VAV3, VCAM1
>207
ABCA4, ABCB1, ACSS2, ADAM23, ADAMTS6, AGK, AKAP9, ANKRD30A, ANKS1B, ATRX, BMPR2, BRAF, C12orf42, CAMKMT, CASP8,
CCDC7, CD22, CDH10, CDH2, CFH, CNTLN, CYP19A1, CYP1A1, CYP3A5, DDIT4, DNAJC13, DYSF, EN1, EPHX1, EPHX2, ERBB3, ERBB4,
ESRRG, FRAS1, GRIK2, GRM8, GSTM2, HCN1, HMGCR, HMGN5, IL1RAPL1, ITGAX, KCNH8, KCTD16, KDM6A, KRT6B, KRTAP9-9,
LINGO2, LMO1, LRRK2, MAST4, MB21D2, MMP2, NCOA3, NUDT13, PARD3, PDPN, PDS5B, PHACTR1, PRKAG2, PTPN22, PTPRD,
ROBO2, SIL1, SOD1, STAG2, SYK, TCHH, TEK, TNFSF11, UTRN, VAV3, VPS13B, WWTR1, ZNF608
>208
ANK3, CD28, CLN3, CTNND2, FAT4, ITGA8, LRRK2, NPHP1, PEX5, SGCZ, SYNE2
>209
ADH1C, ALDH5A1, CHRNA7, GABRA2, GABRB1, GABRB3, GABRG2, GRM5, HTR3B, SLC6A3
>210
ACOT7, ALDH5A1, ANK3, CHRNA7, CRYM, CTSB, CYP3A5, DAB1, DISC1, ERBB3, ERBB4, GABRA2, GABRB1, GABRB3, GABRG2,
GRID1, GRM4, GRM5, HMGCR, NELL1, PRKAG2, RIT2, SLC6A3, THOP1
>211
ABCA4, adenosine triphosphate, ADGRV1, AHI1, ASIC2, CASP8, CFH, CLN3, EYS, HGSNAT, HMCN1, MERTK, MFSD8, NPHP1, PRPF8,
RAB28, SOD1
>212
ABCA13, ABCA4, ABCB1, ACADSB, AHI1, ANK3, ANKRD30A, ARHGAP24, BRAF, CCDC7, CFH, CNTLN, CNTN5, CTNNA3, CTNND2,
CUBN, CWC27, CYP1A1, DNAH11, DNAH14, DYSF, EPHX1, EPHX2, FAT4, FRAS1, GRIK2, HIVEP1, IL15, KDM6A, MERTK, MMP2,
NEK11, OSBPL1A, OTOL1, PHACTR1, PKHD1, PPP2R2B, PRPF8, PXDNL, RPL11, SGCZ, SLC22A20, SLC24A3, SPAG17, STAG2, STAM,
SYNE2, TEK, THSD7B, TLN2, TNFSF11, ULK4, VPS13B, YTHDC2, ZBTB49, ZFPM2
>213
adenosine triphosphate, ARHGAP24, ATRX, BMPR2, CASR, CDH13, CDH2, CTSB, CYBB, CYP19A1, DDAH1, EPHX2, FRAS1, HSD17B4,
KRT6B, MERTK, MMP2, NCOA3, PROS1, RAB28, RILPL2, RNASE10, SMOC2, TBC1D32, TEK, TNFSF11, VCAM1
>214
ERBB3, GABRA2, GABRB1, GABRB3, GABRG2
>215
ABCB1, ANK3, BMPR2, BRAF, CASP8, CD28, CDH2, CPA4, CTSB, CYBB, CYP19A1, CYP3A5, DCC, DOCK4, EN1, ERBB3, ERBB4, ESRRG,
FAF1, FUT8, HYOU1, ICOS, IL15, KLHL3, mir-548, MMP2, NCOA3, NEK11, NFKB1, PARD3, PLOD2, PRPF8, SQSTM1, TEK, TNFSF11,
VAV3, VCAM1
>216
CASR, GABRA2, GABRB1, GABRB3, GABRG2, HMGCR, MMP2, PDE11A, PDE4D, PVT1, SLC6A3
>217
ADH1C, ALDH5A1, CHRNA7, GABRA2, GABRB1, GABRB3, GABRG2, GRM5, HTR3B, PDE11A, PDE4D, SLC6A3
>218
ADH1C, ALDH5A1, CHRNA7, GABRA2, GABRB1, GABRB3, GABRG2, GRM5, HTR3B, PDE4D, SLC6A3
>219
BMPR2, DCC, ERBB4, GABRB3, GRID2, ROBO2, SOD1, TENM2, VAV3, VAX1, VCAM1
>220
CHRNA7, CYP1A1, DAB1, DNAI1, GABRA2, GABRB1, GABRB3, GABRG2, OPHN1, ULK4
>221
ABCB1, ACSL4, adenosine triphosphate, AGK, ANO3, ASIC2, BMPR2, BRAF, CASP8, CASR, CD28, CDH13, CLN3, CRHR1, CTSB, CWC27,
CYP19A1, CYP1A1, CYP3A5, DCC, EGR2, ERBB3, ERBB4, ESRRG, FOXN3, FUT8, GABRA2, GALNT2, GPC5, HMGCR, HPGD, HYAL1,
ICOS, IL15, IQGAP2, KLHL3, LMO1, LUZP2, MMP2,
NCOA3, NFKB1, NRG3, PDE4D, PDS5B, phosphate, PLOD2, PPP2R2B, PPT1, PRMT6, PRPF8, PTPN22, RAPGEF2, SOD1, STAG2, SYK,
TEK, TNFSF11, UTRN, UVRAG, VAV3, VCAM1, WWTR1
>222
CASP8, CD22, CD28, CYBB, IL15
>223
ABCB1, ACADSB, ALOX5, ANKRD30A, CD22, CD28, CHRNA7, CLN3, CYBB, CYP3A5, DYSF, FAM172A, GABRA2, GABRB1, GABRB3,
GABRG2, HMGCR, MERTK, NFKB1, PCTP, PDE11A, PDE4D, PLOD2, PRPF8, PTPRN2, PVT1, RAB28, SLC6A3, STARD3NL, SUCLG2,
TRIM5, XKR4, YTHDC2
>224
CHRNA7, GABRA2, GABRB1, GABRB3, GABRG2, GIF, GRM5, HMGCR, SLC6A3
>225
ADGRB3, ATRX, BRAF, CAPN3, CNTNAP2, ERBB4, GABRA2, KDM6A, LRRK2, MERTK, PDPN, PTPRD, RAPGEF2, SLCO3A1, STAG2,
TUSC3, VAV3, VCAM1
>226
ABCA4, ANO3, BMPR2, BRAF, CASR, CNTN5, CRYM, CYP19A1, DYSF, EGR2, EPHX2, ERBB4, EVC, FGF14, GABRG2, HCN1, HMGCR,
KLHL3, LARS2, LBR, LRRK2, PLOD2, PPP2R2B, PROS1, PRPF8, SLC6A3, SMOC2, SOD1, SQSTM1, SYNE2, TMC1
>227
ABCA13, ABCA4, ABCB1, ACADSB, ANK3, ANKRD30A, ARHGAP24, BRAF, CCDC7, CFH, CNTLN, CNTN5, CTNNA3, CTNND2, CUBN,
CWC27, CYP1A1, DNAH11, DNAH14, DYSF, EPHX1, EPHX2, FAT4, FRAS1, GRIK2, HIVEP1, IL15, KDM6A, MERTK, MMP2, NEK11,
OSBPL1A, OTOL1, PHACTR1, PKHD1, PPP2R2B, PRPF8, PXDNL, RPL11, SGCZ, SLC22A20, SLC24A3, SPAG17, STAG2, STAM, SYNE2,
TIISD7B, TLN2, TNFSF11, ULK4, VPS13B, YTIIDC2, ZBTB49, ZFPM2
>228
ABCB1, ALOX5, DCC, GRM8, HPGD, NELL1, NFKB1, NKAIN2, PDE4D, PPP2R2B, SLC6A3, TBC1D32, TENM4
>229
adenosine triphosphate, CNTNAP2, DISC1, EN1, GABRB3, GRID1, GRM8, OPIIN1, PDE11A
>230
ACADS, ACADSB, ACSL4, adenosine triphosphate, ADH1C, ALOX5, CYP19A1, CYP1A1, CYP3A5, ESRRG, HPGD, HSD17B4, IL15, PEX5,
PPARGC1A, PRKAG2
>231
CASR, GABRA2, GABRB3, GABRG2, HTR3B, SLC6A3
>232
BRAF, ERBB3, ERBB4, NEK11, NFKB1, TEK
>233
ABCB1, ALOX5, DCC, GABRB3, GRM8, HPGD, MERTK, MMP2, NELL1, NFKB1, NKAIN2, PDE4D, PPP2R2B, SLC6A3, TBC1D32, TENM4
>234
ACADSB, AHI1, ATRX, CYP19A1, CYP1A1, FRAS1, GABRA2, GABRB1, GABRB3, GABRG2, HMGCR, HSD17B4, ITGA8, LARS2, NPHP1,
PKHD1, WDPCP, WWTR1, ZFPM2
>235
adenosine triphosphate, ALOX5, ARHGEF4, BMPR2, BRAF, CASR, CTNND2, CTSB, CYBB, CYP19A1, DCC, DOCK4, ERBB3, FUT8, GALNT2,
GPC5, HMGCR, HPGD, HYAL1, HYOU1, ICOS, IL15, IQGAP2, ITGAM, LRRK2, MMP2, NCOA3, PDE4D, phosphate, PLCL1, PPARGC1A,
PPP2R2B, SQSTM1, TSC22D1, TUSC3, UVRAG, VAV3
>236
ADCY8, CHRNA7, CYBB, GABRA2, GABRB1, GABRB3, GABRG2, HMGCR, HYOU1, NFKB1, PDE4D, PROS1, SLC6A3, TNFSF11, VCAM1
>237
ABCB1, ADCY8, adenosine triphosphate, CASP8, CDH2, CHRNA7, CRYM, EGR2, EPHX2, ESRRG, FGF14, FOXN3, GABRA2, GABRB1,
GABRB3, GABRG2, GPR6, GRIK2, HMGCR, ITGAM, MERTK, NFKB1, PCTP, phosphate, PLOD2, PPARGC1A, PPT1, SLC6A3, SOD1,
WWTR1, XK
>238
GABRA2, GABRB1, GABRB3, GABRG2, HMGCR, SYK, VAV3
>239
ADGRB3, ANK3, BRAF, CDH2, CNTNAP2, CRHR1, CTNND2, CYP19A1, DAB1, DCC, DDAH1, EPHA7, ERBB4, FUT8, GRID2, GRM4,
GRM5, IL1RAPL1, LRRK2, NFKB1, OPHN1, PARD3, PTPN20, RAPGEF2, RAPSN, RIT2, ROBO2, SOD1, TENM4
>240
ADGRV1, AGBL4, ANK3, CASP8, CRHR1, CTSB, CYBB, ERBB3, GABRB3, GRID1, GRID2, HSD17B4, LRRK2, NFKB1, PPARGC1A, PPT1,
SOD1, SQSTM1
>241
BRAF, CFH, CHRNA7, CLN3, CNTNAP2, DNM3, EPHA7, GRID2, LHX8, LRRK2, NFKB1, OPHN1, PARD3, PDS5B, PPARGC1A, RAPSN,
ROBO2, SOD1, SYNE2, UTRN, XK
>242
CD22, DISC1, GABRB3, PDE11A, PPARGC1A, SLC6A3
>243
ATRX, CYP19A1, CYP1A1, GABRA2, GABRB1, GABRB3, GABRG2, HSD17B4, LARS2, ZFPM2
>244
AGBL4, ANK3, CTSB, GRID2, HSD17B4, LRRK2, PPT1, SOD1
>245
AGBL4, ANK3, CTSB, GRID2, HSD17B4, PPT1
>246
ABCB1, ANK3, BMPR2, BRAF, CASP8, CD28, CDH2, CFH, CPA4, CTSB, CYBB, CYP19A1, CYP3A5, DCC, DOCK4, EN1, ERBB3, ERBB4,
ESRRG, FAF1, FUT8, HYOU1, ICOS, IL15, KLHL3, mir-548, MMP2, NCOA3, NEK11, NFKB1, PARD3, PLOD2, PRPF8, SQSTM1, TEK,
TNFSF11, VAV3, VCAM1, ZFPM2
>247
ABCA10, BRAF, CD22, CYP1A1, CYP3A5, HMCN1, IL15, LMO1, NCOA3, PARD3, PTPRD, SLC6A3, SYK, TEK
>248
adenosine triphosphate, ANKS1B, CASR, CNTNAP2, GRIK2, GRM5, OPHN1
>249
ABCA4, adenosine triphosphate, ADGRV1, AHI1, ASIC2, CASP8, CFH, CLN3, EYS, HGSNAT, HMCN1, MERTK, MFSD8, NPHP1, PRPF8,
RAB28
>250
ADCY8, ATRX, BRAF, CYP1A1, DCC, ERBB3, EYS, KDM6A, LRRK2
>251
ADAMTS6, CD22, CD28, CFH, CTSB, CXCL13, CYBB, DYM, ERBB3, FGF14, FSTL4, HMGCR, ICOS, ITGAX, MERTK, NFKB1, NRG3,
PPP2R2B, PTPN22, PXDNL, RTP4, SGCZ, VCAM1
>252
ACSL4, adenosine triphosphate, AGK, AKAP9, ALOX5, ARHGAP24, ARHGEF4, BMPR2, BRAF, CASP8, CASR, CD22, CD28, CDH13, CDH2,
CFH, CHRNA7, CNTNAP2, CTNND2, CTSB, CXCL13, CYBB, CYP19A1, CYP1A1, DAB1, DCC, DDIT4, DISC1, DNAH11, DNAI1, DOCK4,
EGR2, EN1, ERBB3, ERBB4, FAF1, FUT8, GALNT2, GRID2, HYAL1, HYOU1, ICOS, IL15, ITGA8, ITGAM, ITGAX, KRT6B, LDHC, LRRK2,
MERTK, MMP2, NCOA3, NFKB1, PARD3, PDPN, PEX5, PHACTR1, PLCL1, PRMT6, PROS1, PTPN12, RAB28, RAPGEF2, ROBO2,
SEMA6D, SOD1, SQSTM1, SYK, SYNE2, TEK, TNFSF11, TUSC3, UTRN, VAV3, VAX1, VCAM1, WDPCP, WWTR1
>253
CHRNA7, CRHR1, CYBB, EPHX1, FUT8, GABRA2, GABRB1, GABRB3, GABRG2, HMGCR, PDE11A, PDE4D
>254
adenosine triphosphate, ANKS1B, ASIC2, CASR, CDH2, CHRNA7, CNTNAP2, FGF14, GABRG2, GRID2, GRIK2, GRM4, GRM5, GRM8,
HTR3B, NFKB1, OPHN1, RAPSN, RIT2
>255
BRAF, CDH2, CFH, CHRNA7, CLN3, CNTNAP2, DNM3, EPHA7, GRID2, GRM5, LHX8, LRRK2, NFKB1, OPHN1, PARD3, PDS5B,
PPARGC1A, RAPSN, ROBO2, SOD1, SYNE2, UTRN, XK
>256
ABCB1, ACADSB, ALOX5, ANKRD30A, CD22, CD28, CHRNA7, CLN3, CYBB, CYP3A5, DYSF, FAM172A, GABRA2, GABRB1, GABRB3,
GABRG2, HMGCR, NFKB1, PCTP, PDE11A, PDE4D, PLOD2, PRPF8, PTPRN2, PVT1, RAB28, SLC6A3, STARD3NL, SUCLG2, TRIM5,
XKR4, YTHDC2
>257
CASR, GABRA2, GABRB1, GABRB3, GABRG2, HTR3B, SLC6A3
>258
ABCB1, adenosine triphosphate, CASP8, CDH2, CHRNA7, CRYM, EGR2, EPHX2, ESRRG, FGF14, FOXN3, GABRA2, GABRB1, GABRB3,
GABRG2, GPR6, GRIK2, HMGCR, ITGAM, MERTK, NFKB1, PCTP, phosphate, PLOD2, PPARGC1A, PPT1, SLC6A3, WWTR1, XK
>259
ABCB1, ACADSB, ALOX5, ANKRD30A, CD22, CD28, CHRNA7, CLN3, CTSB, CYBB, CYP3A5, DYSF, FAM172A, GABRA2, GABRB1,
GABRB3, GABRG2, HMGCR, ITGAM, MERTK, NFKB1, PCTP, PDE11A, PDE4D, PLOD2, PRPF8, PTPRN2, PVT1, RAB28, RIT2, SLC6A3,
STARD3NL, SUCLG2, TRIM5, XK, XKR4, YTHDC2
>260
ABCA12, ABCB1, ACADS, adenosine triphosphate, ADGRL2, AGK, AGXT, AHI1, ALOX5, CASR, CES3, CFH, CHRNA7, CRHR1, ERBB3,
GRID2, GRM4, GRM8, ICOS, P3II1, PCTP, PLCL1, PPT1, PTPRN2, SLC6A3, SOD1, STXBP5L, SYK
>261
ADCY8, ADH1C, ATRX, BRAF, CYP1A1, DCC, ERBB3, EYS, KDM6A, LRRK2, MMP2
>262
ALDII5A1, GABRA2, GABRB1, GABRB3, GABRG2, SLCGA3
>263
ABCC6, BMPR2, BRAF, CDH2, CFH, CTSB, CYBB, DDAH1, EPHX2, HPGD, IL15, MERTK, MMP2, PDPN, PKHD1, PROS1, PTPN12,
RAPGEF2, SYK, TEK, VAV3
>264
CAPN3, CASP8, CDH2, COL19A1, CTNNA3, CYBB, CYP19A1, DYSF, ERBB3, MMP2, PDPN, PPARGC1A, TLN2, UTRN, VAV3, VCAM1, XK,
ZFPM2
>265
GABRB3, GABRG2, IICN1
>266
ERBB3, ERBB4, TNFSF11
>267
ABCA12, BARX1, BMPR2, DCC, DNM3, EGR2, EN1, ERBB3, FAT4, GABRB3, IIPGD, IISD17B4, KDMGA, LIIX8, MMP2, NFKB1, PDPN,
PDS5B, PPARGC1A, PROS1, RAPSN, ROBO2, SC5D, SYK, SYNE2, VAX1, WDPCP
>268
AH11, ANK3, CLN3, EN1, ERBB3, GABRB3, GRID2, IL1RAPL1, MERTK, PPAKGC1A
>269
ALDH5A1, CASP8, DAB1, ERBB4, GABRA2, GABRB1, GABRB3, GABRG2, HMGCR, ITPR2, SOD1, SQSTM1, SUSD1, WWTR1
>270
ABCA4, ABCB1, ADGKB3, ATRX, BRAF, CADM2, CDH2, CFH, COL19A1, CTSB, CUBN, CYP19A1, DCC, EPHA7, EKBB3, ERBB4, ESRRG,
FANCI, HMGCR, HYAL1, IQGAP2, KDM6A, LMO1, LRRK2, MMP2, MTUS2, NFKB1, OSBPL1A, PDS5B, phosphate, PKHD1, PRPF8,
PTPN12, PTPRD, ROBO2, SLC24A3, STAG2, SYK, TEK, UTRN, VAV3, VCAM1, ZFPM2
>271
CHRNA7, GABRA2, GABRB1, GABRB3, GABRG2, GLRA2, SLC6A3
>272
BMPR2, BRAF, CASP8, CD2AP, CDH2, CFH, CTNNA3, CYBB, DDAH1, DNAH11, ERBB3, ERBB4, FAT4, HPGD, IL15, MERTK, MMP2,
PARD3, PDPN, PDS5B, PPARGC1A, PROS1, RAPGEF2, SYK, TEK, VAV3, VCAM1, ZFPM2
>273
GABRA2, GABRB1, GABRB3, GABRG2, GRM5, SLC6A3
>274
adenosine triphosphate, ANK3, CDH2, CHRNA7, CLN3, GRID2, GRIK2, GRM5, HCN1, KCNE1, OPHN1
>275
adenosine triphosphate, BMPR2, CYP19A1, EGR2, EN1, MMP2, NFKB1, NPR3, OSCAR, PDS5B, PLCL1, SOD1, SQSTM1, TNFSF11, WWTR1
>276
GABRA2, GABRB1, GABRB3, GABRG2
>277
BMPR2, BRAF, CASP8, CD2AP, CDH2, CFH, CTNNA3, CYBB, DDAH1, DNAH11, ERBB3, FAT4, HPGD, IL15, MERTK, MMP2, PARD3,
PDPN, PDS5B, PPARGC1A, PROS1, SYK, TEK, VAV3, VCAM1, ZFPM2
>278
ALOX5, CHRNA7, GABRA2, GABRB1, GABRB3, GABRG2, HMGCR
>279
adenosine triphosphate, CXCL13, ITGAM, ITGAX, MMP2, VCAM1
>280
ADCY8, adenosine triphosphate, AHI1, CLN3, CRHR1, CYP19A1, DAB1, EN1, FGF14, GABRB3, GRID2, GRM5, LRRK2, PDE11A,
PPARGC1A, PPT1, SLC6A3, SOD1, SQSTM1
>281
CXCL13, CYP19A1, ICOS, IL15, NFKB1, PDPN, PTPN22, TNFSF11
>282
ALOX5, CHRNA7, CYBB, CYP19A1, GABRA2, GABRB1, GABRB3, GABRG2, HYOU1, NFKB1, PPARGC1A, SLC6A3, SOD1
>283
GABRA2, GABRB1, GABRB3, GABRG2, SLC6A3, ULK4
>284
BARX1, CRHR1, CYP19A1, DMRTA1, ERBB3, ERBB4, FAT4, FRAS1, GRID2, IL15, IMMP2L, ITGA8, LHX8, LRRK2, MMP2, NFKB1,
PKHD1, PTPN12, PTPRN2, RAPGEF2, ROBO2, SOD1, TNFSF11, TSC22D1, VCAM1, WDPCP, WWTR1
>285
CLN3, DYSF, GABRB3, GABRG2, PPARGC1A, PPT1, STAM
>286
adenosine triphosphate, CASR, GRM5, ITGAM, ITPR2, PDE4D, TNFSF11
>287
adenosme triphosphate, ATRX, BRAF, CASP8, CDH2, CHRNA7, CLN3, CRHR1, CTSB, CYBB, DCC, EN1, EPHA7, EPHX2, ERBB3, GABRB3,
GRID2, GRIK2, GRM4, GRM5, HYOU1, ITPR2, LRRK2, NFKB1, PPARGC1A, PPP2R2B, PPT1, RAPSN, RNF19A, SLC6A3, SOD1, STAM,
>288
ABCA4, ABCB1, ACOT7, ACSL4, ACSS2, adenosine triphosphate, ADH1C, AGK, ALOX5, BMPR2, CASP8, CASR, CLN3, CRHR1, CYP19A1,
CYP1A1, DAB1, DLD, EPHX2, ERBB4, ESRRG, GRM5, HMGCR, HPGD, IL15, JAZF1, MMP2, NFKB1, PARD3, PCTP, PPARGC1A, PRKAG2,
SOD1, SYK
>289
CD28, ICOS, MERTK, VCAM1
>290
GABRA2, GABRB1, GABRB3, GABRG2
>291
ANKRD30A, CCDC178, CNTLN, DYM, FRAS1, GLRA2, GRM5, KDM6A, KRTAP9-8, SPAG17, TRIM36, ULK4, VAV3
>292
ABCA4, ABCC6, CFH
>293
ALOX5, GABRA2, GABRB1, GABRB3, GABRG2, SLC6A3
>294
ABCA4, ABCB1, ADH1C, CYP3A5, PCTP, PPT1
>295
ABCB1, ACADSB, ALOX5, ANKRD30A, CD22, CD28, CHRNA7, CLN3, CYBB, CYP3A5, DYSF, FAM172A, GABRA2, GABRB1, GABRB3,
GABRG2, HMGCR, NFKB1, PCTP, PDE11A, PDE4D, PLOD2, PRPF8, PTPRN2, PVT1, RAB28, SLC6A3, STARD3NL, SUCLG2, XKR4,
YTHDC2
>296
ABCB1, GABRA2, GABRB1, GABRB3, GABRG2, HTR3B, PDE11A
>297
ABCA4, ADGRV1, AGBL4, AHI1, ANK3, CASP8, CTSB, DYSF, ERBB3, GABRB3, GRID1, GRID2, HSD17B4, LRRK2, MERTK, NFKB1,
PPARGC1A, PPT1, SOD1
>298
ADCY8, adenosine triphosphate, CDH2, CHRNA7, CRHR1, CYBB, CYP19A1, ERBB4, GABRG2, GRIK2, GRM5, ITGA8, SLC6A3, SOD1, VAV3
>299
DCC, EGR2, EN1, ERBB3, ERBB4, GABRB3, SOD1, VAX1
>300
ADCY8, ATRX, BRAF, DCC, ERBB3, EYS, KDMGA, LRRK2
>301
ATRX, CYP19A1, GABRA2, GABRB1, GABRB3, GABRG2, HSD17B4, LARS2, ZFPM2
>302
ACSS2, adenosine triphosphate, ALOX5, IIMGCR, IIPGD, IISD17B4
>303
ABCB1, CHRNA7, GABRA2, GABRB1, GABRB3, GABRG2, GLRA2, PDE4D, SLC6A3
>304
ACSL4, BRAF, CYP19A1, ERBB4, FAT4, PARD3, PKIID1, ROBO2, WDPCP, WWTR1
>305
ABCA12, PDS5B
>306
MERTK, PROS1
>307
HMGCR, ITGAM
>308
ALDII5A1, CLN3
>309
ALDH5A1, CLN3
>310
BRAF, TSC22D1
>311
CNTNAP2, CRHR1, CYP19A1, ERBB4, GABRG2, GRIK2, GRM5, LRRK2, NFKB1, OPHN1, PPT1, PTPRN2, ROBO2, SLC6A3, TPST1
>312
ABCB1, CHRNA7, GABRA2, GABRB1, GABRB3, GABRG2, HMGCR, SLC6A3
>313
GABRA2, GABRB1, GABRB3, GABRG2, HTR3B
>314
FAT4, PARD3, PKHD1, ROBO2, WDPCP, WWTR1
>315
ABCA10, ADAM23, AGBL4, AHI1, AKAP9, ANK3, BMPR2, BRAF, CASR, CCDC7, CNTLN, CTNNA3, CUBN, DNAH14, DYM, DYSF, ERBB3,
FAT4, FOXP2, FSTL4, GRIK2, GRM8, IQGAP2, ITGAX, KCNH8, KCTD16, LRRK2, MTUS2, OSBPL1A, PHACTR1, RAPGEF2, RAPSN,
ROBO2, SH3RF3, TCHH, VPS13B
>316
ADGRV1, ALDH5A1, ANK3, CHRNA7, CLN3, CNTNAP2, CRYM, EGR2, GABRA2, GABRB1, GABRB3, GABRG2, GRIK2, GRM5, HCN1,
HYOU1, PLCL1, PPT1, PTPRN2, STAM
>317
CLN3, CNTNAP2, DAB1, DCC, OPHN1, PPARGC1A, PPT1, ROBO2, TENM2, ULK4, VAX1
>318
GABRA2, GABRB1, GABRB3, GABRG2
>319
adenosine triphosphate, CD28, ICOS, IL15
>320
GABRA2, GABRB1, GABRB3, GABRG2
>321
ALDH5A1, GABRA2, GABRB1, GABRB3, GABRG2
>322
GABRA2, GABRB1, GABRB3, GABRG2, SLC6A3
>323
adenosine triphosphate, CYP19A1, EGR2, MMP2, NFKB1, NPR3, OSCAR, PLCL1, SOD1, SQSTM1, TNFSF11
>324
ABCB1, ADH1C, ALDH5A1, CAPN3, GASP8, CHRNA7, CTNNA3, DAB1, DNAJC13, ERBB4, GABRA2, GABRB1, GABRB3, GABRG2,
HMGCR, ITPR2, LRRK2, MERTK, PDE4D, SLC24A3, SLC6A3, SOD1, SQSTM1, SUSD1, WWTR1
>325
ABCB1, adenosine triphosphate, ADH1C, CAPN3, CASP8, CDH2, CHRNA7, CRYM, CTNNA3, DNAJC13, EGR2, EPHX2, ESRRG, FOXN3,
GABRA2, GABRB1, GABRB3, GABRG2, GPR6, GRIK2, HMGCR, ICOS, ITGAM, LRRK2, MERTK, NFKB1, PCTP, PDE4D, phosphate, PLOD2,
PPARGC1A, RAPSN, SLC24A3, SLC6A3, SOD1, SQSTM1, WWTR1
>326
ACSL4, adenosine triphosphate, BMPR2, CASP8, CD28, CLN3, CYP19A1, CYP3A5, ERBB3, ERBB4, ESRRG, HPGD, NCOA3, PDS5B,
phosphate, PPP2R2B, PRMT6, SOD1, SYK, TEK, UTRN, WWTR1
>327
CD28, CXCL13, EGH2, ICOS, PDPN, TNFSF11
>328
adenosine triphosphate, CXCL13, CYP19A1, EGR2, GRID1, ITGAM, MMP2, NFKB1, NPR3, OSCAR, PLCL1, SOD1, SQSTM1, TNFSF11
>329
ACSL4, adenosine triphosphate, AGK, AKAP9, ALOX5, ARHGAP24, ARHGEF4, BMPR2, BRAF, CASP8, CASR, CD22, CD28, CDH13, CDH2,
CFH, CHRNA7, CNTNAP2, CTSB, CXCL13, CYBB, CYP19A1, CYP1A1, DAB1, DCC, DDIT4, DISC1, DOCK4, EGR2, ERBB3, ERBB4, FAF1,
FUT8, GALNT2, HYAL1, HYOU1, ICOS, IL15, ITGA8,
ITGAM, ITGAX, KRT6B, LRRK2, MERTK, MMP2, NCOA3, NFKB1, PDPN, PEX5, PHACTR1, PLCL1, PRMT6, PROS1, PTPN12,
RAB28, RAPGEF2, ROBO2, SEMA6D, SOD1, SYK, SYNE2, TEK, TNFSF11, TUSC3, VAV3, VAX1, VCAM1, WDPCP, WWTR1
>330
GABRA2, GABRB1, GABRB3, GABRG2
>331
ERBB3, ERBB4, NFKB1, TNFSF11
>332
ADGRB3, EN1, GRID2, GRM4, HCN1
>333
ABCB1, adenosine triphosphate, ADH1C, CAPN3, CASP8, CDH2, CHRNA7, CRYM, CTNNA3, DNAJC13, EGR2, EPHX2, ESRRG, FOXN3,
GABRA2, GABRB1, GABRB3, GABRG2, GPR6, GRIK2, HMGCR, ITGAM, LRRK2, MERTK, NFKB1, PCTP, PDE4D, phosphate, PLOD2,
PPARGC1A, SLC6A3, SOD1, WWTR1
>334
CHRNA7, CLN3, CTSB, DAB1, ERBB3, GRID1, MERTK, OPHN1, PEX5, PPARGC1A, PPT1, PROS1
>335
ADGRV1, AGBL4, ANK3, CTSB, ERBB3, GABRB3, GRID1, GRID2, HSD17B4, LRRK2, NFKB1, PPARGC1A, PPT1, SOD1, SQSTM1
>336
CASR, CYP3A5, HMGCR, ICOS, MMP2
>337
CASP8, ERBB3, ERBB4, PDPN, TEK
>338
GABRA2, GABRB1, GABRB3, GABRG2, SLC6A3
>339
adenosine triphosphate, CYP19A1, FOXP2, IL1RAPL1, STAM, VWC2
>340
ABCA12, AGXT, AKAP9, BMPR2, BRAF, CCDC178, CCDC7, DOCK4, DYSF, ERBB3, GRIK2, KCTD16, PDS5B, ULK4, UTRN, UVRAG
>341
BRAF, CDH2, DAB1, FOXP2, LHX8, LRRK2, PARD3, ROBO2, ULK4, VAX1
>342
ACADS, ACADSB, ACSL4, adenosine triphosphate, ADH1C, CYP19A1, ESRRG, HPGD, HSD17B4, PEX5, PPARGC1A, PRKAG2
>343
GRID1, NFKB1, SOD1
>344
ERBB3, ERBB4, NFKB1
>345
CLN3, CYBB, PROS1, SOD1
>346
CLN3, LRRK2, MFSD8, PPT1
>347
GABRA2, GABRB1, GABRB3, GABRG2
>348
BRAF, ERBB3, ERBB4, NEK11
>349
ANK3, CHRNA7, GABRA2, GABRB1, GABRB3, GABRG2, SLC6A3
>350
ABCA12, ABCB1, ACADS, adenosine triphosphate, AGK, ALOX5, CES3, CFH, CRHR1, GKM4, PCTP, PTPKN2, SOD1
>351
ALDH5A1, GABRA2, GABRB1, GABRB3, GABRG2, GRM5, HTR3B, SLC6A3
>352
CRHR1, ERBB4, GABRG2, GRM5, NFKB1, PTPRN2
>353
ABCA12, BMPR2, CRHR1, FAT4, FUT8, HYAL1, ITGA8, LRRK2, PDPN, PPARGC1A, SYK
>354
adenosine triphosphate, CASR, CHRNA7, GRIK2, NFKB1, PKHD1, SLC24A3, SOD1, TNFSF11, XK
>355
ATRX, CHRNA7, ERBB3, PPARGC1A
>356
GABRA2, GABRB1, GABRB3, GABRG2
>357
ACSL4, GRIK2, OPHN1, TUSC3
>358
CAPN3, CASP8, CDH2, COL19A1, CTNNA3, CYBB, DYSF, ERBB3, MMP2, PDPN, PPARGC1A, TLN2, VAV3, VCAM1, XK, ZFPM2
>359
ABCB1, ACSL4, ALOX5, BARX1, CASP8, CD2AP, CFH, CLN3, CRHR1, CYBB, CYP19A1, CYP1A1, ERBB3, ESRRG, FAT4, HYAL1, ICOS,
IL15, IQGAP2, ITGA8, LRRK2, MERTK, NFKB1, PCTP, PEX5, PKHD1, PPARGC1A, PPT1, PTPN22, ROBO2, SC5D, SOD1, TNFSF11,
WDPCP, ZFPM2
>360
CD2AP, DNAJC13, LRRK2, NFKB1, SOD1, TMC1
>361
ANK3, CDH2, DCC, EGR2, ERBB4, OPHN1, ROBO2, TENM2, VAV3, VAX1
>362
GABRA2, GABRB1, GABRB3, GABRG2, HMGCR, MMP2, SLC6A3, SOD1, VCAM1, ZFPM2
>363
CASR, CYP1A1, GABRA2, GABRB1, GABRB3, GABRG2, HMGCR, MMP2, PDE11A, PDE4D, phosphate, PVT1, SLC6A3, TNFSF11
>364
GABRA2, GABRB1, GABRB3, GABRG2, GRM5, SLC6A3
>365
ABCA4, CFH, CLN3, MERTK, PPT1, SOD1, TENM2
>366
GABRB1, PDE4D, SLC6A3
>367
adenosine triphosphate, DAB1, LIPG
>368
ADGRV1, AGBL4, ANK3, CRHR1, CTSB, CYBB, ERBB3, GABRB3, GRID1, GRID2, HSD17B4, LRRK2, NFKB1, PPARGC1A, PPT1, SOD1,
SQSTM1
>369
BMPR2, CASP8, CD2AP, CDH2, CTNNA3, CYBB, DNAH11, ERBB3, ERBB4, FAT4, MMP2, PARD3, PDPN, PDS5B, PPARGC1A, TEK, VAV3,
VCAM1, ZFPM2
>370
ADCY8, CHHNA7, CRHR1, EHBB4, GABRG2, GRM5, GRM8, HSD17B4, NFKB1, OPHN1, PTPRN2, SLC6A3
>371
CD22, CHRNA7, IL15, NFKB1
>372
ADGRL2, ALDH5A1, CASP8, CD2AP, CHRNA7, CTNNA3, CTSB, DLGAP2, DYSF, GABRA2, GABRB1, GABRB3, GABRG2, GRM4, GRM5,
GRM8, HMGCR, LDLRAP1, MMP2, MS4A4E, PROS1, SC5D, SLC6A3, SOD1, VPS13B
>373
ADGRL2, ALDH5A1, CASP8, CD2AP, CHRNA7, CTNNA3, CTSB, DLGAP2, DYSF, GABRA2, GABRB1, GABRB3, GABRG2, GRM4, GRM5,
GRM8, HMGCR, LDLRAP1, MMP2, MS4A4E, PROS1, SC5D, SLC6A3, SOD1, SQSTM1, VPS13B
>374
ADGRV1, AGBL4, ANK3, CTSB, ERBB3, GABRB3, GRID1, GRID2, HSD17B4, LRRK2, NFKB1, PPARGC1A, PPT1, SOD1
>375
ACADS, ACOT7, ADAM23, AGXT, AKAP8, ALDH5A1, ANKRD30A, ATRX, BRAF, CADM2, CUBN, CWC27, CYP1A1, DNAH14, EPHX1,
ERBB3, ERBB4 FAM122C, GABRA2, GABRB1, GABRB3, GABRG2, HMCN1, HMGCR, HSD17B4, ITGAX, KDM6A, KRTAP9-4, LRRK2,
MAST4, MMP2, NCOA3, PKHD1, PLOD2, PRKAG2, ROBO2, SCG3, STAG2, TEK, XK, YTHDC2
>376
ABCB1, ALOX5, CHRNA7, GABRA2, GABRB1, GABRB3, GABRG2
>377
ABCB1, GABRA2, GABRB1, GABRB3, GABRG2, HTR3B, PDE11A
>378
ALOX5, CHRNA7, CYBB, CYP19A1, GABRA2, GABRB1, GABRB3, GABRG2, HYOU1, LRRK2, NFKB1, PPARGC1A, SLC6A3, SOD1
>379
ABCA4, ABCB1, ADH1C, CYP3A5, PCTP
>380
adenosine triphosphate, ANKS1B, GRIK2, GRM5, OPHN1
>381
BRAF, CHRNA7, CNTNAP2, CRHR1, CYBB, GRM5, HCN1, HMGCR, ITGA8, OPHN1, SLC6A3
>382
CHRNA7, CLN3, CTSB, DAB1, ERBB3, MERTK, OPHN1, PEX5, PPARGC1A, PPT1, PROS1
>383
CHRNA7, GABRA2, GABRB1, GABRB3, GABRG2, SLC6A3
>384
ABCA4, ADGRV1, AGK, BRAF, EYS, IIGSNAT, MERTK, MFSD8, NPIIP1, OCA2, PRPF8, RAB28, SOD1, VAX1
>385
adenosine triphosphate, FOXP2, IL1RAPL1, STAM
>386
AIII1, AKAP9, BBS5, BBS9, BRAF, DISC1, PKIID1, TBC1D32, ULK4, WDPCP, WWTR1
>387
CYP19A1, GABRA2, GABRB1, GABRB3, GABRG2
>388
AKAP9, CDII13, DOCK4, ITGAM, NFKB1, PTPN12, WWTR1
>389
PPT1, TENM2
>390
CD28, ICOS
>391
CTSB, SQSTM1
>392
CD28, ICOS
>393
CHRNA7, NCOA3
>394
ABCB1, BRAF
>395
ITGAM, ITGAX
>396
EGR2, FAF1
>397
SOD1, SQSTM1
>398
CYP19A1, ESRRG
>399
ABCC6, XYLT1
>400
ERBB3, ERBB4
>401
BMPR2, CASP8, CD2AP, CDH2, CTNNA3, CYBB, DNAH11, ERBB3, FAT4, MMP2, PARD3, PDPN, PDS5B, PPARGC1A, TEK, VAV3,
VCAM1, ZFPM2
>402
DCC, EGR2, EN1, ERBB3, ERBB4, GABRB3, SOD1, SYNE2, VAX1
>403
ACSL4, CYP19A1, ERBB4, FAT4, PARD3, PKHD1, ROBO2, WDPCP
>404
AHI1, BMPR2, ERBB3, ERBB4, PTPRD, SYK, TEK
>405
CLN3, CNTNAP2, DAB1, DCC, ERBB3, OPHN1, PPARGC1A, PPT1, ROBO2, TENM2, ULK4, VAX1
>406
adenosine triphosphate, CDH2, KCNE1
>407
ABCA4, adenosine triphosphate, CASP8, CFH, HMCN1
>408
CTSB, GRID2, PPARGC1A, PPT1, SOD1, SQSTM1
>409
ANO3, CDH13, CWC27, GPC5, LUZP2, SYK
>410
ALOX5, BMPR2, BRAF, CASP8, CHRNA7, CRHR1, CYP19A1, GABRA2, GABRB1, GABRB3, GABRG2, HMGCR, MERTK, PDE4D, PROS1,
SPRED2, SYK, TEK, ULK4, ZFPM2
>411
CASP8, CDH2, CTNNA3, CYBB, ERBB3, PDPN, PPARGC1A, VAV3, VCAM1, ZFPM2
>412
ABCB1, ADH1C, CAPN3, CHRNA7, CTNNA3, DNAJC13, GABRA2, GABRB1, GABRB3, GABRG2, LRRK2, PDE4D, SLC6A3, SOD1
>413
ABCA12, ACADS, ANKRD30A, BRAF, CCDC105, CPNE8, DCC, DDIT4, DISC1, DNAH11, ESRRG, FAM184A, FOXP2, FRAS1, FUT8,
GABRB1, HIVEP1, HMCN1, HSD17B4, KCTD8, NCOA3, PTPN12, PTPRD, RPL11, SH3RF3, TCHH, TEK, TENM2, THSD7B, TNFSF11,
TRHDE, ULK4, VCAM1
>414
BMPR2, CASP8, CTNNA3, ERBB3, ERBB4, MMP2, PDPN, PPARGC1A, TEK, VCAM1, ZFPM2
>415
GABRA2, GABRB1, GABRB3, GABRG2, HCN1, HTR3B, SLC6A3
>416
ABCA12, ABCA4, ABCB1, BRAF, CFH, CLN3, CYP19A1, ERBB3, IQGAP2, KRT6B, LRRK2, MERTK, PCTP, PDPN, SOD1, SYK, SYNE2,
TEK, TNFSF11
>417
EGR2, ERBB4, GABRB3, SOD1
>418
GABRA2, GABRB1, GABRB3, GABRG2
>419
ABCA12, CASR, CYP19A1, EPHX2, HMGCR, HSD17B4, LDLRAP1, LIPG, PPARGC1A, SC5D, SOD1
>420
BMPR2, BRAF, CFH, CYBB, DDAH1, HPGD, IL15, MERTK, MMP2, PDPN, PKHD1, PROS1, SYK, TEK, VAV3
>421
adenosine triphosphate, CD28, ICOS, IL15, MERTK
>422
adenosine triphosphate, CASP8, CDH2, CRYM, EGR2, EPHX2, ESRRG, FOXN3, GABRA2, GABRB1, GABRB3, GABRG2, GPR6, GRIK2,
HMGCR, ITGAM, MERTK, NFKB1, PCTP, phosphate, PLOD2, PPARGC1A, SLC6A3, WWTR1, XK
>423
CTSB, EPHX2, PPARGC1A, PXDNL, SIL1, SOD1, SQSTM1
>424
CASR, EPHX2, HMGCR, LDLRAP1, LIPG, SC5D, SOD1
>425
ARHGAP24, CD28, CDH2, GRID2, ITGAM, MMP2, NELL1, TLN2, TNFSF11, VCAM1
>426
AHI1, ERBB3, GABRB3
>427
ADCY8, PDE11A, SLC6A3
>428
ABCA4, ITGAM, ITGAX
>429
NFKB1, SOD1, TMC1
>430
ADH1C, HPGD, HSD17B4
>431
ERBB4, GRID2, PKHD1
>432
ABCA12, BMPR2, CRHR1, FAT4, FUT8, HYAL1, ITGA8, LRRK2, MMP2, PDPN, PDS5B, PPARGC1A, SC5D, SYK, ULK4
>433
ANKRD30A, CCDC178, CFH, CNTLN, DYM, ESRRG, FRAS1, GLRA2, GRM5, KDM6A, KRTAP9-8, SPAG17, TRIM36, ULK4, VAV3
>434
ALOX5, CHRNA7, CYBB, GABRA2, GABRB1, GABRB3, GABRG2, HYOU1, SLC6A3
>435
ADCY8, ADGRB3, ATRX, BRAF, CAPN3, CNTNAP2, ERBB4, FAM184A, FGF14, GABRA2, HCN1, HMCN1, HYOU1, KDM6A, LRRK2,
MERTK, PDPN, PTPRD, RAPGEF2, SLCCA3, SLCO3A1, STAG2, TEK, TRIM5, TUSC3, VAV3, VCAM1, VPS13B
>436
ABCB1, BARX1, CLN3, COL19A1, CYBB, CYP19A1, CYP1A1, ERBB3, FAT4, HYAL1, ICOS, IQGAP2, KRT6B, MERTK, NFKB1, PCTP,
PEX5, PKHD1, PPARGC1A, PPT1, SC5D, TNFSF11, WDPCP, ZFPM2
>437
ICOS, ITGAM, SYK, VCAM1
>438
GABRA2, GABRG2, HTR3B, SLC6A3
>439
ADGRB3, ATRX, BRAF, CAPN3, CNTNAP2, DLEU1, ERBB4, GABRA2, KDM6A, LRRK2, MERTK, MMP2, PDPN, PTPRD, PVT1, RAPGEF2,
SLCO3A1, STAG2, TUSC3, VAV3, VCAM1
>440
BRAF, CHRNA7, DAB1, GRM4, GRM5, LRRK2, PARD3, PRKAG2, RAPGEF2, SOD1, SYK, TNFSF11, WWTR1
>441
ADCY8, CHRNA7, GABRA2, GABRB1, GABRB3, GABRG2, HMGCR, PDE4D, SLC6A3, VCAM1
>442
CDH2, CNTNAP2, CRHR1, CTNND2, CYP19A1, EMB, EPHX2, ERBB4, GRM5, MMP2, NFKB1, OPHN1, PTPN12, PTPN20, RAPGEF2,
RAPSN, ROBO2, SOD1, TEK
>443
ABCB1, GABKA2, GABRB1, GABRB3, GABRG2, SLC6A3
>444
FOXP2, GABRA2, GABRB1, GABRB3, GABRG2
>445
CHRNA7, GABRA2, GABRB1, GABRB3, GABRG2, HMGCR, SLC6A3
>446
ACADS, ACOT7, ADAM23, AGXT, AKAP9, ALDH5A1, ANKRD30A, ATRX, BRAF, CADM2, CUBN, CWC27, CYP1A1, DCC, DNAH14,
EPHX1, ERBB3, ERBB4, FAM122C, GABRA2, GABRB1, GABRB3, GABRG2, HMCN1, HMGCR, HSD17B4, ITGAX, KDM6A,
KRTAP9-4, LRRK2, MAST4, MMP2, NCOA3, PKHD1, PLOD2, PRKAG2, ROBO2, SCG3, STAG2, TEK, UTRN, XK, YTHDC2
>447
ABCB1, ALOX5, BMPR2, CASP8, CD22, CDH13, CFH, CHRNA7, CYBB, CYP19A1, CYP1A1, IL15, ITGAM, NFKB1, RAPGEF2, SOD1,
VCAM1
>448
ABCB1, ACSL4, adenosine triphosphate, AGK, ALOX5, ANK3, ANO3, ARHGAP24, ASIC2, BARX1, BMPR2, BRAF, CASP8, CASR, CD22,
CD28, CD2AP, CDH13, CDH2, CHRNA7, CLN3, CRHR1, CTNND2, CTSB, CWC27, CYBB, CYP19A1, CYP1A1, CYP3A5, DCC, DDAH1,
DDIT4, DISC1, DLD, DOCK4, EGR2, EPHX2, ERBB3, ERBB4,
ESRRG, FAT4, FOXN3, FUT8, GABRA2, GALNT2, GPC5, GPR6, GRID2, GRIK2, GRM4, GRM5, HMGCR, HMGN5, HPGD, HYAL1,
ICOS, IL15, IQGAP2, ITGAM, ITGAX, KDM6A, KLHL3, LBR, LDLRAP1, LIPG, LMNTD1, LMO1, LRRK2, LUZP2, MERTK, MMP2, NCOA3,
NELL1, NFKB1, NPR3, NRG3, OCA2, OSBPL1A, P3H1, PDE4D, PDPN, PDS5B, phosphate, PKHD1, PLOD2, PPARGC1A, PPP2R2B, PPT1,
PRMT6, PROS1, PRPF8, PTPN12, PTPN22, RAB28, RAPGEF2, RAPSN, RIT2, ROBO2, SOD1, SQSTM1, STAG2, STAM, SYK, TEK, TLN2,
TNFSF11, TSC22D1, TUSC3, UTRN, UVRAG, VAV3, VAX1, VCAM1, VPS13B, WWTR1, ZBTB49
>449
GABRA2, GABRB1, GABRB3, GABRG2, PPARGC1A, SLC6A3
>450
CHRNA7, GABRG2, GRID2
>451
ACSS2, adenosine triphosphate, HMGCR
>452
CYBB, ITGAM, NFKB1
>453
GABRA2, GABRB1, GABRB3, GABRG2, TNFSF11
>454
CHRNA7, CNTNAP2, DISC1, ERBB4, GABRB3, GABRG2, OPHN1, PPARGC1A, SLC6A3
>455
adenosine triphosphate, ARHGAP24, BMPR2, CDH13, CDH2, CTSB, CYBB, DDAH1, EPHX2, KDM6A, MERTK, MMP2, NCOA3, PROS1,
RAB28, SMOC2, TEK, VCAM1
>456
ADGRV1, CASP8, DCC, GABRB3, GRID1, GRID2
>457
ADAMTS6, BMPR2, CAPN3, CASP8, DNAH11, DNAI1, ERBB3, ERBB4, ESRRG, FAT4, KDM6A, MMP2, PARD3, PDPN, PPARGC1A,
PTPN12, RAPGEF2, TBC1D32, TEK, UTRN, VCAM1, ZFPM2
>458
AHI1, BMPR2, BRAF, CD22, CD28, CDH10, CNTNAP2, CTNND2, DLEU1, FAT4, HIVEP1, IL15, LRRK2, PDE4D, PLOD2, STAG2, SYK,
SYNE2, TEK, TMC1, TNFSF11, TRHDE
>459
ABCA12, ACADS, ANKRD30A, BRAF, CCDC105, CPNE8, DCC, DDIT4, DISC1, DNAH11, ESRRG, FAM184A, FOXP2, FRAS1, GABRB1,
HIVEP1, HMCN1, KCTD8, NCOA3, PTPRD, RPL11, SH3RF3, TCHH, TENM2, THSD7B, TRHDE, VCAM1
>460
CDH2, CTSB, MERTK, MMP2, SQSTM1, VAV3, VCAM1
>461
ALDH5A1, GABRA2, GABRB1, GABRB3, GABRG2
>462
GABRA2, GABRB1, GABRB3, GABRG2
>463
CD22, CD28, ICOS, IL15
>464
CLN3, RNF19A, SIL1, SOD1
>465
GABRA2, GABRB1, GABRB3, GABRG2
>466
ANKS1B, CASR, CHRNA7, CNTNAP2, GRIK2, GRM5, OPHN1, RAPSN
>467
ADCY8, adenosine triphosphate, ADGRB3, ATRX, BRAF, CAPN3, CASP8, CNTNAP2, ERBB4, FAM184A, FGF14, FOXP2, GABRA2, IICN1,
HMCN1, HYOU1, KCNH8, KDM6A, LRRK2, MERTK, PDPN, PTPRD, RAPGEF2, SLC6A3, SLCO3A1, STAG2, TEK, TRIM5, TUSC3, VAV3,
VCAM1, VPS13B
>468
ERBB3, ERBB4
>469
ERBB3, ERBB4
>470
ERBB3, ERBB4
>471
ADGRV1, SOD1
>472
ADCY8, GRM5
>473
ABCB1, SLC6A3
>474
ABCB1, adenosine triphosphate
>475
ITGAM, ITGAX
>476
ERBB3, ERBB4
>477
ABCB1, CYP3A5
>478
CYP19A1, CYT3A5
>479
ERBB3, ERBB4
>480
ERBB3, ERBB4
>481
ERBB3, ERBB4
>482
ERBB3, ERBB4
>483
ERBB3, ERBB4
>484
ERBB3, ERBB4
>485
ERBB3, ERBB4
>486
ERBB3, ERBB4
>487
ERBB3, ERBB4
>488
ERBB3, ERBB4
>489
ERBB3, ERBB4
>490
ERBB3, ERBB4
>491
ERBB3, ERBB4
>492
ERBB3, ERBB4
>493
ERBB3, ERBB4
>494
ERBB3, ERBB4
>495
ERBB3, ERBB4
>496
ERBB3, ERBB4
>497
ERBB3, ERBB4
>498
ERBB3, ERBB4
>499
ERBB3, ERBB4
>500
ERBB3, ERBB4
>501
ERBB3, ERBB4
>502
ERBB3, ERBB4

LENGTHY TABLES
The patent application contains a lengthy table section. A copy of the table is available in electronic form from the USPTO web site (http://seqdata.uspto.gov/?pageRequest=docDetail&DocID=US20180187190A1). An electronic copy of the table will also be available from the USPTO upon request and payment of the fee set forth in 37 CFR 1.19(b)(3).

1. Eukaryotic expression vector comprising a eukaryotic CRISPR, preferably a mammalian or a plant CRISPR.

2. The eukaryotic expression vector of claim 1, wherein the CRISPR is one of the CRISPR sequences of SEQ ID NO: 1-SEQ ID NO: 11297.

3. The eukaryotic expression vector of claim 1, wherein the mammalian CRISPR is a human CRISPR and wherein said CRISPR sequence is one of the sequences of SEQ ID NO: 1-10141.

4. The eukaryotic expression vector of claim 1, wherein the CRISPR is under control of an endogenous promoter.

5. The eukaryotic expression vector of claim 1, wherein the CRISPR is under control of a heterologous promoter.

6. Method of assaying the effect of a eukaryotic CRISPR on the gene regulation of a eukaryotic cell comprising the steps of:
a. transforming or transfecting said cell with a eukaryotic expression vector of claim 1;
b. allowing the CRISPR to be transcribed;
c. measuring the transcriptome of said cell.

7. The method of claim 6, wherein said CRISPR is a human CRISPR and said cell is a human cell.

8. The method of claim 6 in which the effect on gene regulation is measured by comparing the total RNA of the cell before and after expression of the CRISPR.

9. Method of assaying the transcription of a eukaryotic CRISPR comprising the steps of:
a. transforming or transfecting a cell with a eukaryotic expression vector of claim 4;
b. subjecting said cell to a stimulus;
c. measuring the transcriptome of said cell.

10. The method of claim 9, wherein said stimulus is a chemical stimulus.

11. The method of claim 9, wherein said stimulus is a physical stimulus.

12-13. (canceled)

14. A kit comprising the vector of claim 1 and instructions for performing a method to study gene regulation in eukaryotes.

15. The kit of claim 14, wherein the eukaryotes are mammals.

16. The kit of claim 14, wherein the eukaryotes are humans.
